(19) United States
(12) Patent Application Publication
(10) Pub. No.: US 2024/0238425 A1
(43) Pub. Date: Jul. 18, 2024

(54) HSD17B13 Inhibitors and/or Degraders

(71) Applicant: Pfizer Inc., New York, NY (US)

(72) Inventors: Jan Antoinette Cusi Romero Adams, Arlington, MA (US); Yotam Ashkenazi, Groton, CT (US); Matthew Frank Brown, Stonington, CT (US); Jason Kenneth Dutra, Gales Ferry, CT (US); Michelle Renee Garnsey, Newton, MA (US); Xinjun Hou, Winchester, MA (US); Jisun Lee, Mystic, CT (US); Yajing Lian, Waterford, CT (US); Deane Milford Nason, II, Norwich, CT (US); Steven Victor O'Neil, East Lyme, CT (US); Amanda Brooke Pecora, Groton, CT (US); Alistair Dean Richardson, Quaker Hill, CT (US); Matthew Forrest Sammons, Quincy, MA (US); Yang Wang, West Newton, MA (US); Ann Sorrentino Wright, Old Lyme, CT (US); Jun Xiao, East Lyme, CT (US); Lei Zhang, Auburndale, MA (US); Liying Zhang, Lexington, MA (US)

(73) Assignee: Pfizer Inc., New York, NY (US)

(21) Appl. No.: 18/541,015

(22) Filed: Dec. 15, 2023

Related U.S. Application Data
(60) Provisional application No. 63/387,759, filed on Dec. 16, 2022.

(51) Int. Cl. A61K 47/55; A61K 45/06

(52) U.S. Cl. CPC A61K 47/55; A61K 45/06

(57) ABSTRACT

Described herein are 3-fluoro-4-hydroxybenzamide-containing inhibitors and/or degraders, and pharmaceutical compositions containing 3-fluoro-hydroxybenzamide-containing inhibitors and/or degraders. In some embodiments, the 3-fluoro-4-hydroxybenzamide-containing compounds of the disclosure can be used to treat a condition, for example, nonalcoholic fatty liver disease and nonalcoholic steatohepatitis.

### BACKGROUND OF THE INVENTION

Hydroxysteroid 17β-dehydrogenase13 (HSD17B13) is a hepatic lipid droplet-associated steroid dehydrogenase family enzyme. From 2018 to present, multiple human genetic variants of HSD17B13 have been identified as protective against NASH progression, where these human variants resulted in reduced hepatic inflammation, ballooning, and fibrosis. Abul-Husn et al., 2018 reported a truncation variant was over-enriched in individuals with simple steatosis and under-enriched in NASH and NASH+fibrosis individuals, implying its protection against disease progression. Abul-Husn, et al., “Protein-Truncating HSD17B13 Variant and Protection from Chronic Liver Disease”, N Engl J Med 2018; 378:1096-1106. Later that year, a second truncation variant was reported by Kozlitina et al. with reduced allele frequency in blacks and Hispanics with chronic liver disease. Kozlitina, et al., “HSD17B13 and Chronic Liver Disease in Blacks and Hispanics”, N Engl J Med 2018; 379:1876-1877. In 2019, a coding variant, P260S, was found by Ma et al. to be associated with reduced inflammation and ballooning. HSD17B13 expression has been shown to be significantly upregulated in humans with non-alcoholic fatty liver disease (NAFLD). Ma, et al., “17-Beta Hydroxysteroid Dehydrogenase 13 Is a Hepatic Retinol Dehydrogenase Associated With Histological Features of Nonalcoholic Fatty Liver Disease”, Hepatology 2019; 69(4):1504-1519. Murine models placed on pro-NASH diets have also demonstrated upregulation of the HSD17B13. As such, inhibition or degradation of HSD17B13 enzymatic activity is hypothesized to slow or prevent the progression of liver diseases such as nonalcoholic fatty liver diseases (NAFLDs) including NASH (nonalcoholic steatohepatitis), hepatic inflammation, fibrosis, cirrhosis, and development of hepatocellular carcinoma.

Although there has been some early research related to HSD17B13, there remains a need for pharmaceutical agents that have HSD17B13 inhibiting and/or degrading activity. HSD17B13 inhibitors and/or degraders can be used in the treatment, prevention, or diminution of the manifestations of the maladies described herein.

### SUMMARY OF THE INVENTION

In some embodiments, disclosed herein is a compound of Formula I:

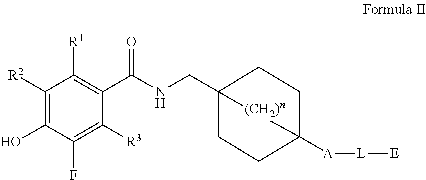

wherein:
A is —NH—C(O)—, —C(O)—, or heteroaryl, wherein heteroaryl has 1, 2, 3, or 4 heteroatoms selected from O, N, and S, and wherein A is optionally substituted with one or two R⁴;
R¹, R², and R³ are each independently selected from H and fluoro;
R⁴ is selected from oxo, hydroxyl, chloro, fluoro, (C₁-C₆)alkyl, (C₁-C₆)alkoxy, (C₁-C₆)fluoroalkyl, (C₃-C₆)cycloalkyl, and heterocyclyl, wherein the heterocyclyl has 1, 2, or 3 heteroatoms selected from O and N;
n is 0, 1, or 2;
L is a linker; and
E is an E3 ubiquitin ligase binder,
or a pharmaceutically acceptable salt thereof.

In some embodiments, disclosed herein is a compound of the structure:

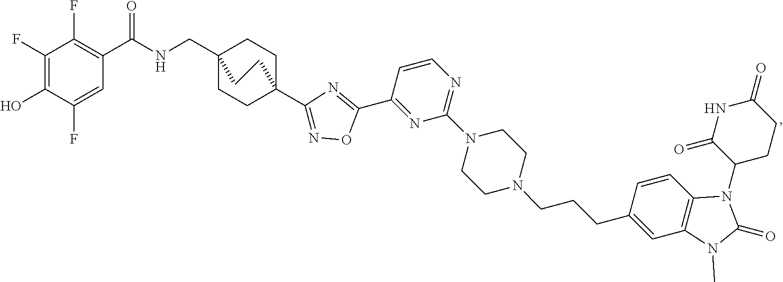 [SMILES: Cn1c(=O)n(C2CCC(=O)NC2=O)c2ccc(CCCN3CCN(c4nccc(-c5nc([C@]67CC[C@](CNC(=O)c8cc(F)c(O)c(F)c8F)(CC6)CC7)no5)n4)CC3)cc21]

or a pharmaceutically acceptable salt thereof. Further disclosed herein is a pharmaceutically acceptable salt of N-{[4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)bicyclo[2.2.2]octan-1-yl]methyl}-2,3,5-trifluoro-4-hydroxybenzamide. Also disclosed herein is N-{[4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)bicyclo[2.2.2]octan-1-yl]methyl}-2,3,5-trifluoro-4-hydroxybenzamide, hydrochloride salt. In some embodiments, disclosed herein is N-{[4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)bicyclo[2.2.2]octan-1-yl]methyl}-2,3,5-trifluoro-4-hydroxybenzamide.

In some embodiments, disclosed herein is a pharmaceutical composition comprising a therapeutically effective amount of a compound of the disclosure or a pharmaceutically acceptable salt thereof, and at least one pharmaceutically acceptable carrier, vehicle, or diluent. Further disclosed herein is a pharmaceutical combination composition comprising: a therapeutically effective amount of a composition comprising: a first compound, said first compound being a compound of Formula II or a pharmaceutically acceptable salt of said compound; a second compound, said second compound being an anti-diabetic agent; a non-alcoholic steatohepatitis treatment agent, a non-alcoholic fatty liver disease treatment agent or an anti-heart failure treatment agent and a pharmaceutical carrier, vehicle, or diluents.

In some embodiments, disclosed herein is a method for treating a condition, comprising administering to a subject in need thereof a therapeutically effective amount of the compound of the disclosure, or a pharmaceutically acceptable salt thereof, wherein the condition is selected from the group consisting of: fatty liver, non-alcoholic fatty liver disease, non-alcoholic steatohepatitis, non-alcoholic steatohepatitis with liver fibrosis, non-alcoholic steatohepatitis with cirrhosis, non-alcoholic steatohepatitis with cirrhosis, hepatocellular carcinoma, alcoholic fatty liver disease, alcoholic steatohepatitis, hepatitis B, hepatitis C, biliary cirrhosis, kidney renal clear cell carcinoma, head and neck squamous cell carcinoma, colorectal adenocarcinoma, mesothelioma, stomach adenocarcinoma, adrenocortical carcinoma, kidney papillary cell carcinoma, cervical and endocervical carcinoma, bladder urothelial carcinoma, lung adenocarcinoma, Type I diabetes, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, restenosis after angioplasty, peripheral vascular disease, intermittent claudication, post-prandial lipemia, metabolic acidosis, ketosis, arthritis, diabetic retinopathy, macular degeneration, cataract, diabetic nephropathy, glomerulosclerosis, chronic renal failure, diabetic neuropathy, skin and connective tissue disorders, foot ulcerations and ulcerative colitis, endothelial dysfunction and impaired vascular compliance, kidney disease, end-stage kidney disease, chronic kidney disease at risk of progression, and maple syrup urine disease.

In some embodiments, disclosed herein is a method of reducing development of a condition, comprising administering to a subject in need thereof a therapeutically effective amount of a compound of the disclosure or a pharmaceutically acceptable salt thereof, wherein the condition is selected from the group consisting of: liver cirrhosis, cirrhotic decompensation, progression to model of end-stage liver disease (MELD), liver transplant, liver-related death, and hepatocellular carcinoma.

Disclosed herein is a compound of the disclosure or a pharmaceutically acceptable salt thereof, for use in the treatment of fatty liver, non-alcoholic fatty liver disease, non-alcoholic steatohepatitis, non-alcoholic steatohepatitis with liver fibrosis, non-alcoholic steatohepatitis with cirrhosis, non-alcoholic steatohepatitis with cirrhosis, hepatocellular carcinoma, alcoholic fatty liver disease, alcoholic steatohepatitis, hepatitis B, hepatitis C, biliary cirrhosis, kidney renal clear cell carcinoma, head and neck squamous cell carcinoma, colorectal adenocarcinoma, mesothelioma, stomach adenocarcinoma, adrenocortical carcinoma, kidney papillary cell carcinoma, cervical and endocervical carcinoma, bladder urothelial carcinoma, lung adenocarcinoma, Type I diabetes, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, restenosis after angioplasty, peripheral vascular disease, intermittent claudication, post-prandial lipemia, metabolic acidosis, ketosis, arthritis, diabetic retinopathy, macular degeneration, cataract, diabetic nephropathy, glomerulosclerosis, chronic renal failure, diabetic neuropathy, skin and connective tissue disorders, foot ulcerations and ulcerative colitis, endothelial dysfunction and impaired vascular compliance, kidney disease, end-stage kidney disease, chronic kidney disease at risk of progression, or maple syrup urine disease.

In some embodiments, disclosed herein is a use of a compound of the disclosure or a pharmaceutically acceptable salt thereof, as a medicament. In some embodiments, disclosed herein is a use of a compound of the disclosure or a pharmaceutically acceptable salt thereof, in the manufacture of a medicament for the treatment of fatty liver, non-alcoholic fatty liver disease, non-alcoholic steatohepatitis, non-alcoholic steatohepatitis with liver fibrosis, non-alcoholic steatohepatitis with cirrhosis, non-alcoholic steatohepatitis with cirrhosis, hepatocellular carcinoma, alcoholic fatty liver disease, alcoholic steatohepatitis, hepatitis B, hepatitis C, biliary cirrhosis, kidney renal clear cell carcinoma, head and neck squamous cell carcinoma, colorectal adenocarcinoma, mesothelioma, stomach adenocarcinoma, adrenocortical carcinoma, kidney papillary cell carcinoma, cervical and endocervical carcinoma, bladder urothelial carcinoma, lung adenocarcinoma, Type I diabetes, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, restenosis after angioplasty, peripheral vascular disease, intermittent claudication, post-prandial lipemia, metabolic acidosis, ketosis, arthritis, diabetic retinopathy, macular degeneration, cataract, diabetic nephropathy, glomerulosclerosis, chronic renal failure, diabetic neuropathy, skin and connective tissue disorders, foot ulcerations and ulcerative colitis, endothelial dysfunction and impaired vascular compliance, kidney disease, end-stage kidney disease, chronic kidney disease at risk of progression, or maple syrup urine disease.

In some embodiments, disclosed herein is a use of a compound of the disclosure or a pharmaceutically acceptable salt thereof, in treating a condition selected from the group consisting of: fatty liver, non-alcoholic fatty liver disease, non-alcoholic steatohepatitis, non-alcoholic steatohepatitis with liver fibrosis, non-alcoholic steatohepatitis with cirrhosis, non-alcoholic steatohepatitis with cirrhosis, hepatocellular carcinoma, alcoholic fatty liver disease, alcoholic steatohepatitis, hepatitis B, hepatitis C, biliary cirrhosis, kidney renal clear cell carcinoma, head and neck squamous cell carcinoma, colorectal adenocarcinoma, mesothelioma, stomach adenocarcinoma, adrenocortical carcinoma, kidney papillary cell carcinoma, cervical and endocervical carcinoma, bladder urothelial carcinoma, lung adenocarcinoma, Type I diabetes, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, restenosis after angioplasty, peripheral vascular disease, intermittent claudication, post-prandial lipemia, metabolic acidosis, ketosis, arthritis, diabetic retinopathy, macular degeneration, cataract, diabetic nephropathy, glomerulosclerosis, chronic renal failure, diabetic neuropathy, skin and connective tissue disorders, foot ulcerations and ulcerative colitis, endothelial dysfunction and impaired vascular compliance, kidney disease, end-stage kidney disease, chronic kidney disease at risk of progression, and maple syrup urine disease. It is to be understood that both the foregoing general description and the following detailed description are exemplary and explanatory only and are not restrictive of the invention, as claimed.

### DETAILED DESCRIPTION OF THE INVENTION

This application may be understood more readily by reference to the following detailed description of exemplary embodiments of the invention and the examples included therein.

It is to be understood that this invention is not limited to specific synthetic methods of making that may of course vary. It is also to be understood that the terminology used herein is for the purpose of describing particular embodiments only and is not intended to be limiting. In this specification and in the claims that follow, reference will be made to a number of terms that shall be defined to have the following meanings:

As used herein in the specification, “a” or “an” may mean one or more. As used herein in the claim(s), when used in conjunction with the word “comprising”, the words “a” or “an” may mean one or more than one. As used herein “another” may mean at least a second or more.

The term “about” refers to a relative term denoting an approximation of plus or minus 10% of the nominal value it refers, in one embodiment, to plus or minus 5%, in another embodiment, to plus or minus 2%. For the field of this disclosure, this level of approximation is appropriate unless the value is specifically stated to require a tighter range.

The term “and/or” means one or more. For example, “X and/or Y” shall be understood to mean either “X and Y” or “X or Y” and shall be taken to provide explicit support for both meanings or for either meaning. Similarly, when more than 2 expressions are listed, such as in “X, Y and/or Z”, it shall be understood to mean either i) “X and Y”, “X, Y and Z”, “X and Z”, or “Y and Z”, or ii) “X or Y or Z” and shall be taken to provide explicit support for all meanings.

Any open valency appearing on a carbon, oxygen, sulfur, or nitrogen atom in the structures disclosed herein indicates the presence of a hydrogen, unless indicated otherwise.

The term C₁-Cₓ includes C₁-C₂, C₁-C₃ . . . C₁-Cₓ. By way of example only, a group designated as “C₁-C₄” indicates that there are one to four carbon atoms in the moiety, i.e., groups containing 1 carbon atom, 2 carbon atoms, 3 carbon atoms, or 4 carbon atoms. For example, “C₁-C₄alkyl” indicates that there are one to four atom carbons in the alkyl group, i.e., the alkyl group is selected from methyl, ethyl, propyl, isopropyl, n-butyl, isobutyl, sec-butyl, and t-butyl.

The term “bicyclic ring system” denotes two rings that are fused each other via a common single or double bond (annelated bicyclic ring system), via a sequence of three or more common single atoms (bridged bicyclic ring system), or via a common single atom (spiro bicyclic ring system). Bicyclic ring systems can be saturated, partially saturated, unsaturated, or aromatic. Bicyclic rings can comprise heteroatoms selected from N, O, and S.

The term “bridged” refers to any ring structure with two or more rings that contain a bridge connecting two bridgehead atoms. The bridgehead atoms are defined as atoms that are part of the skeletal framework of the molecule and which are bonded to three or more other skeletal atoms. The bridgehead atoms can be C, N, or P. The bridge can be a single atom or a chain of atoms that connects two bridgehead atoms. For example, a bridged ring system can be cycloalkyl or heterocycloalkyl.

The term “fused” refers to any ring structure described herein which is fused to an existing ring structure. When the fused ring is a heterocyclyl ring or a heteroaryl ring, any carbon atom on the existing ring structure that becomes part of the fused heterocyclyl ring or the fused heteroaryl ring may be replaced with one or more N, S, and O atoms. Non-limiting examples of fused heterocyclyl rings include 6-5 fused heterocycle, 6-6 fused heterocycle, 5-6 fused heterocycle, 5-5 fused heterocycle, 7-5 fused heterocycle, and 5-7 fused heterocycle. Non-limiting examples of fused heteroaryl rings include 6-5 fused heteroaryl, 6-6 fused heteroaryl, 5-6 fused heteroaryl, 5-5 fused heteroaryl, 7-5 fused heteroaryl, and 5-7 fused heteroaryl.

The terms “carbocyclic” or “carbocycle” refer to a ring or ring system where the atoms forming the backbone of the ring are all carbon atoms. The term is distinguished from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. For example, carbocycle includes cycloalkyl and aryl.

The term “alkyl” refers to an acyclic, saturated hydrocarbon group of the formula CnH₂ₙ₊₁, which may be linear or branched. The carbon atom content of alkyl and various other hydrocarbon-containing moieties is indicated by a prefix designating a lower and upper number of carbon atoms in the moiety, that is, the prefix Cᵢ-Cⱼ; indicates a moiety of the integer “i” to the integer “j” carbon atoms, inclusive. Thus, for example, C₁-C₃ alkyl refers to alkyl of one to three carbon atoms, inclusive. For example, an alkyl comprising up to 10 carbons is referred to as C₁-C₁₀ alkyl. For example, an alkyl comprising up to 6 carbon atoms is referred to as C₁-C₆ alkyl. Alkyls (and other moieties defined herein) comprising other numbers of carbon atoms are represented similarly. Examples of such groups include, but are not limited to, methyl, ethyl, n-propyl, isopropyl, n-butyl, sec-butyl, isobutyl and t-butyl. Alkyl groups may be optionally substituted or unsubstituted, as further defined herein.

The term “heteroalkyl” refers to an alkyl group in which one or more skeletal atoms of the alkyl are selected from an atom other than carbon, for example, N, S, O, or combinations thereof. Representative examples of heteroalkyl include, but are not limited to, —NH—, —N(alkyl)-, —N(aryl)-, —S—, —S(═O)—, or —S(═O)₂—, thioether, —OCH₂OMe, —OCH₂CH₂OH, —OCH₂CH₂OMe, or —OCH₂CH₂OCH₂CH₂NH₂, or combinations thereof. Heteroalkyl can be attached to the rest of the molecule at a carbon atom of the heteroalkyl. Heteroalkyl can also be attached to the rest of the molecule at a heteroatom of the heteroalkyl.

The term “haloalkyl” refers to an alkyl group wherein at least one of the hydrogen atoms of the alkyl group has been replaced by at least one of the same or different halogen atoms. For example, “fluoroalkyl” means an alkyl as defined herein substituted with one, two or three fluoro atoms. Exemplary (C₁)fluoroalkyl compounds include fluoromethyl, difluoromethyl and trifluoromethyl; exemplary (C₂)fluoroalkyl compounds include 1-fluoroethyl, 2-fluoroethyl, 1,1-difluoroethyl, 1,2-difluoroethyl, 1,1,1-trifluoroethyl, 1,1,2-trifluoroethyl, and the like. Examples of fully substituted fluoroalkyl groups (also referred to as perfluoroalkyl groups) include trifluoromethyl (—CF₃) and pentafluoroethyl (—C₂F₅).

“Cycloalkyl” refers to a monocyclic, bridged or fused bicyclic, or polycyclic non-aromatic ring that is fully hydrogenated and has the formula CnH₂ₙ₋₁. Cycloalkyl groups can be spiro-cyclic or bridged compounds. Cycloalkyl groups can be fused with an aromatic system, in which case the cycloalkyl is bonded through a non-aromatic ring carbon atom. Cycloalkyl groups can also be fused with a second cycloalkyl group. Cycloalkyl groups may contain, but are not limited to, 3 to 12 carbon atoms (“C₃-C₁₂ cycloalkyl”), 3 to 8 carbon atoms (“C₃-C₈ cycloalkyl”), 3 to 6 carbon atoms (“C₃-C₆ cycloalkyl”), 3 to 5 carbon atoms (“C₃-C₅ cycloalkyl”) or 3 to 4 carbon atoms (“C₃-C₄ cycloalkyl”). Representative cycloalkyl rings include cyclopropyl, cyclobutyl, cyclopentyl, cyclohexyl, cycloheptyl, and cyclooctyl. Polycyclic cycloalkyl groups include, for example, adamantanyl, 1,2-dihydronaphthalenyl, 1,4-dihydronaphthalenyl, tetraenyl, decalinyl, 3,4-dihydronaphthalenyl-1(2H)-one, spiro[2.2]pentyl, norbornyl, and bicycle[1.1.1]pentyl. A cycloalkyl group can be optionally substituted as defined herein.

“Fluorocycloalkyl” means a nonaromatic cycloalkyl ring as defined herein substituted with one, two or three fluoro atoms. Exemplary (C₃)fluorocycloalkyl compounds include fluorocyclopropyl, difluorocyclopropyl and trifluorocyclopropyl; exemplary (C₄)fluorocycloalkyl compounds include 1-fluorocyclobutyl, 2-fluorocyclobutyl, 1,1-difluorocyclobutyl, 1,2-difluorocyclobutyl, 1,1,1-trifluorocyclobutyl, 1,1,2-trifluorocyclobutyl, and the like.

The term “alkoxy” refers to a straight chain saturated alkyl or branched chain saturated alkyl bonded through an oxy, i.e., —OR^{x}, wherein R^{x} is an alkyl radical as defined above. In some embodiments, the term “alkoxy” refers to alkylene comprising an oxy, i.e., alkylene-O-alkylene, —O— alkylene, or alkylene-O—. Representative alkoxy groups include methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy and octoxy. Alkoxy groups may be optionally substituted or unsubstituted, as further defined herein.

“Fluoroalkoxy” means an alkoxy as defined herein substituted with one, two or three fluoro atoms. Exemplary (C₁)fluoroalkoxy compounds include fluoromethoxy, difluoromethoxy and trifluoromethoxy; exemplary (C₂)fluoroalkyl compounds include 1-fluoroethoxy, 2-fluoroethoxy, 1,1-difluoroethoxy, 1,2-difluoroethoxy, 1,1,1-trifluoroethoxy, 1,1,2-trifluoroethoxy, and the like.

The terms “halo”, “halogen”, and “halide” are used interchangeably herein and refer to bromo, chloro, fluoro or iodo.

“Cyano” refers to a substituent having a carbon atom joined to a nitrogen atom by a triple bond, i.e., —C≡N.

“Hydroxy” refers to an —OH group.

“Oxo” refers to a double bonded oxygen (═O).

When “ene” is added after “yl” at the end a term to form a new term, the new term refers to a diradical formed by removing one hydrogen atom from the original term of which the new term derived. For example, “alkylene” refers to a diradical group formed by removing one hydrogen atom from an alkyl group and that a “methylene” refers to a divalent radical —CH₂-derived from removing one hydrogen atom from methyl, i.e., a straight or branched divalent hydrocarbon chain linking the rest of the molecule to a radical group. Examples of such diradicals include, but are not limited to: alkylene, alkenylene, alkynylene, cycloalkylene, phenylene, heterocyclylene, and heteroarylene, which are derived from alkyl, alkenyl, alkynyl, cycloalkyl, phenyl, heterocyclyl, and heteroarylene. Non-limiting examples of “C₁₋₃ alkylene” include: —CH₂—, —CH(CH₃)—, —CH₂—CH₂—, —CH₂—CH₂—CH₂—, —CH(CH₃)—CH₂—, and —CH(CH₂CH₃)—. For a cyclic moiety, the removal of the hydrogen can occur on any atom of sufficient valency.

“Alkenyl” refers to an alkyl group, as defined herein, refers to aliphatic hydrocarbons having at least one carbon-carbon double bond, including straight chains and branched chains having at least one carbon-carbon double bond. In some embodiments, the alkenyl group has 2 to 6 carbon atoms. In some embodiments, the alkenyl group has 2 to 4 carbon atoms. For example, as used herein, the term “C₂₋₆ alkenyl” means straight or branched chain unsaturated radicals of 2 to 6 carbon atoms, including, but not limited to, ethenyl, 1-propenyl, 2-propenyl (allyl), isopropenyl, 2-methyl-1-propenyl, 1-butenyl, 2-butenyl, and the like, optionally substituted by 1 to 5 suitable substituents. When the compounds of the disclosure contain an alkenyl group, the alkenyl group may exist as the pure E form, the pure Z form, or any mixture thereof.

“Alkynyl” refers to an alkyl group, as defined herein, consisting of at least two carbon atoms and at least one carbon-carbon triple bond. Examples include, but are not limited to, ethynyl, 1-propynyl, 2-propynyl, 1-, 2-, or 3-butynyl, and the like.

“Heterocycloalkyl” or “heterocyclyl” refers to a non-aromatic, saturated ring system containing the specified number of ring atoms and containing at least one heteroatom selected from N, O and S as a ring member, where ring S atoms are optionally substituted by one or two oxo groups (i.e., S(O)_{q}, where q is 0, 1 or 2) and where the heterocycloalkyl ring is connected to the base molecule via a ring atom, which may be C or N. Heterocycloalkyl rings include rings which are monocyclic, spirocyclic, bridged, or fused to one or more other heterocycloalkyl or carbocyclic rings, where such spirocyclic, bridged, or fused rings may themselves be saturated, partially unsaturated or aromatic to the extent unsaturation or aromaticity makes chemical sense, provided the point of attachment to the base molecule is an atom of the heterocycloalkyl portion of the ring system. Heterocycloalkyl rings may contain 1 to 4 heteroatoms selected from N, O, and S(O)_{q} as ring members, or 1 to 2 ring heteroatoms, provided that such heterocycloalkyl rings do not contain two contiguous oxygen or sulfur atoms. Heterocycloalkyl rings may be optionally substituted or unsubstituted, as further defined herein. Such substituents may be present on the heterocyclic ring attached to the base molecule, or on a spirocyclic, bridged or fused ring attached thereto. Heterocycloalkyl rings may include, but are not limited to, 3-8 membered heterocyclyl groups, for example 4-7 or 4-6 membered heterocycloalkyl groups, in accordance with the definition herein. Illustrative examples of heterocycloalkyl rings include, but are not limited to a monovalent radical of oxirane (oxiranyl), thiirane (thiaranyl), aziridine (aziridinyl), oxetane (oxetanyl), thietane (thiatanyl), azetidine (azetidinyl), tetrahydrofuran (tetrahydrofuranyl), tetrahydrothiophene (tetrahydrothiophenyl), pyrrolidine (pyrrolidinyl), tetrahydropyran (tetrahydropyranyl), tetrahydrothiopyran (tetrahydrothiopyranyl), piperidine (piperidinyl), 1,4-dioxane (1,4-dioxanyl), 1,4-oxathiarane (1,4-oxathiaranyl), morpholine (morpholinyl), 1,4-dithiane (1,4-dithianyl), piperazine (piperazinyl), thiomorpholine (thiomorpholinyl), oxepane (oxepanyl), thiepane (thiepanyl), azepane (azepanyl), 1,4-dioxepane (1,4-dioxepanyl), 1,4-oxathiepane (1,4-oxathepanyl), 1,4-oxaazepane (1,4-oxaazepanyl), 1,4-thiazepane (1,4-thiazapanyl), 1,4-diazepane (1,4-diazepanyl), 1,4-dithepane (1,4-dithiepanyl), diooxalanyl, thienyl[1,3]dithianyl, tetrahydroquinonyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxapiperidinyl, 2-oxopyrrolidinyl, oxazolidinyl, quinuclidinyl, thiazolidinyl, trithianyl, thiomorpholinyl, thiamorpholinyl, 1-oxothiomorpholinyl, or 1,1-dioxothiomorpholinyl. Fused bicyclic heterocyclyl groups can comprise a first heterocyclyl group fused to a second heterocyclyl group. Illustrative examples of bridged and fused heterocycloalkyl groups include, but are not limited to a monovalent radical of 1-oxa-5-azabicyclo-[2.2.1]heptane, 3-oxa-8-azabicyclo-[3.2.1]octane, 3-azabicyclo-[3.1.0]hexane, or 2-azabicyclo-[3.1.0]hexane. Illustrative examples of heterospirocyclic compounds include, are but not limited to substituted or unsubstituted spiro[3.4]nonanyl, spiro[3.5]decanyl, spiro[5.4]undecanyl, spiro[4.5]undecanyl, or spiro[5.5]tetradecanyl, wherein the heterospirocyclic compounds comprise at least one heteroatom selected from N, O and S as a ring member.

The term “aromatic” refers to a planar ring having a delocalized r-electron system containing 4n+2π electrons, wherein n is an integer. Aromatics can be optionally substituted. The term “aromatic” includes both monocyclic or fused bicyclic aryl groups (e.g., phenyl, naphthalenyl) and monocyclic or fused bicyclic heteroaryl groups (e.g., pyridinyl, quinolinyl)

“Aryl” refers to a monocyclic, fused bicyclic or polycyclic ring system that contains the specified number of ring atoms, in which all carbon atoms in the ring are of sp² hybridization and in which the pi electrons are in conjugation. Aryl groups may contain, but are not limited to, 6 to 20 carbon atoms (“C₆-C₂₀ aryl”), 6 to 14 carbon atoms (“C₆-C₁₄ aryl”), 6 to 12 carbon atoms (“C₆-C₁₂ aryl”), or 6 to 10 carbon atoms (“C₆-C₁₀ aryl”). Fused aryl groups may include an aryl ring (e.g., a phenyl ring) fused to another aryl ring. Fused aryl rings may also include an aryl ring (e.g., phenyl ring) fused to cycloalkyl. In some embodiments, fused aryl rings can include an aryl ring (e.g., phenyl ring) fused to a heterocyclyl group. In one embodiment, fused aryl rings can include an aryl ring (e.g., phenyl ring) fused to a heteroaryl ring. Examples include, but are not limited to, phenyl, naphthyl, anthracenyl, phenanthrenyl, indanyl, and indenyl. Aryl groups may be optionally substituted, unsubstituted or substituted, as further defined herein.

The term “heteroaryl” refers to monocyclic, heterobiaryl or fused bicyclic or polycyclic ring systems that contain the specified number of ring atoms and include at least one heteroatom selected from N, O and S as a ring member in a ring in which all carbon atoms in the ring are of sp² hybridization and in which the pi electrons are in conjugation. The total number of ring members may be indicated (e.g., a 5- to 10-membered heteroaryl). Heteroaryl groups may contain, but are not limited to, 5 to 20 ring atoms (“5-20 membered heteroaryl”), 5 to 14 ring atoms (“5-14 membered heteroaryl”), 5 to 12 ring atoms (“5-12 membered heteroaryl”), 5 to 10 ring atoms (“5-10 membered heteroaryl”), 5 to 9 ring atoms (“5-9 membered heteroaryl”), or 5 to 6 ring atoms (“5-6 membered heteroaryl”). Heteroaryl rings are attached to the base molecule via a ring atom of the heteroaromatic ring. Thus, either 5- or 6-membered heteroaryl rings, alone or in a fused structure, may be attached to the base molecule via a ring C or N atom. The heteroaryl group can include two fused rings, where at least one of the rings is aromatic and the other is aromatic, saturated, or partially unsaturated and at least one of the fused rings contains the heteroatom. In some embodiments, a heteroaryl ring can be fused to a cycloalkyl ring. In some embodiments, a heteroaryl ring can be fused to an aryl ring. In some embodiments, a first heteroaryl ring can be fused to a second heteroaryl ring. Examples of heteroaryl groups include, but are not limited to, pyrrolyl, furanyl, thiophenyl, pyrazolyl, imidazolyl, isoxazolyl, oxazolyl, isothiazolyl, thiazolyl, triazolyl, oxadiazolyl, thiadiazolyl, tetrazolyl, pyridinyl, pyridizinyl, pyrimidinyl, pyrazinyl, benzofuranyl, benzothiophenyl, indolyl, benzamidazolyl, indazolyl, quinolinyl, isoquinolinyl, purinyl, triazinyl, naphthyridinyl, cinnolinyl, quinazolinyl, quinoxalinyl and carbazolyl. Examples of 5- or 6-membered heteroaryl groups include, but are not limited to, pyrrolyl, furanyl, thiophenyl, pyrazolyl, imidazolyl, isoxazolyl, oxazolyl, isothiazolyl, thiazolyl, triazolyl, pyridinyl, pyrimidinyl, pyrazinyl and pyridazinyl rings. Heteroaryl groups may be optionally substituted, unsubstituted or substituted, as further defined herein.

Illustrative examples of monocyclic heteroaryl groups include, but are not limited to a monovalent radical of pyrrole (pyrrolyl), furan (furanyl), thiophene (thiophenyl), pyrazole (pyrazolyl), imidazole (imidazolyl), isoxazole (isoxazolyl), oxazole (oxazolyl), isothiazole (isothiazolyl), thiazolyl (thiazolyl), 1,2,3-triazole (1,2,3-triazolyl), 1,3,4-triazole (1,3,4-triazolyl), 1-oxa-2,3-diazole (1-oxa-2,3-diazolyl), 1-oxa-2,4-diazole (1-oxa-2,4-diazolyl), 1-oxa-2,5-diazole (1-oxa-2,5-diazolyl), 1-oxa-3,4-diazole (1-oxa-3,4-diazolyl), 1-thia-2,3-diazole (1-thia-2,3-diazolyl), 1-thia-2,4-diazole (1-thia-2,4-diazolyl), 1-thia-2,5-diazole (1-thia-2,5-diazolyl), 1-thia-3,4-diazole (1-thia-3,4-diazolyl), tetrazole (tetrazolyl), pyridine (pyridinyl), pyridazine (pyridazinyl), pyrimidine (pyrimidinyl), or pyrazine (pyrazinyl).

Illustrative examples of fused ring heteroaryl groups include, but are not limited to benzofuran (benzofuranyl), benzothiophene (benzothiophenyl), indole (indolyl), benzimidazole (benzimidazolyl), indazole (indazolyl), benzotriazole (benzotriazolyl), pyrrolo[2,3-b]pyridine (pyrrolo[2,3-b]pyridinyl), pyrrolo[2,3-c]pyridine (pyrrolo[2,3-c]pyridinyl), pyrrolo[3,2-c]pyridine (pyrrolo[3,2-c]pyridinyl), pyrrolo[3,2-b]pyridine (pyrrolo[3,2-b]pyridinyl), imidazo[4,5-b]pyridine (imidazo[4,5-b]pyridinyl), imidazo[4,5-c]pyridine (imidazo[4,5-c]pyridinyl), pyrazolo[4,3-d]pyridine (pyrazolo[4,3-d]pyridinyl), pyrazolo[4,3-c]pyridine (pyrazolo[4,3-c]pyridinyl), pyrazolo[3,4-c]pyridine (pyrazolo[3,4-c]pyridinyl), pyrazolo[3,4-b]pyridine (pyrazolo[3,4-b]pyridinyl), isoindole (isoindolyl), indazole (indazolyl), purine (purinyl), indolizine (indolizinyl), imidazo[1,2-a]pyridine (imidazo[1,2-a]pyridinyl), imidazo[1,5-a]pyridine (imidazo[1,5-a]pyridinyl), pyrazolo[1,5-a]pyridine (pyrazolo[1,5-a]pyridinyl), pyrrolo[1,2-b]pyridazine (pyrrolo[1,2-b]pyridazinyl), imidazo[1,2-c]pyrimidine (imidazo[1,2-c]pyrimidinyl), quinoline (quinolinyl), isoquinoline (isoquinolinyl), cinnoline (cinnolinyl), quinazoline (azaquinazoline), quinoxaline (quinoxalinyl), phthalazine (phthalazinyl), 1,6-naphthyridine (1,6-naphthyridinyl), 1,7-naphthyridine (1,7-naphthyridinyl), 1,8-naphthyridine (1,8-naphthyridinyl), 1,5-naphthyridine (1,5-naphthyridinyl), 2,6-naphthyridine (2,6-naphthyridinyl), 2,7-naphthyridine (2,7-naphthyridinyl), pyrido[3,2-d]pyrimidine (pyrido[3,2-d]pyrimidinyl), pyrido[4,3-d]pyrimidine (pyrido[4,3-d]pyrimidinyl), pyrido[3,4-d]pyrimidine (pyrido[3,4-d]pyrimidinyl), pyrido[2,3-d]pyrimidine (pyrido[2,3-d]pyrimidinyl), pyrido[2,3-b]pyrazine (pyrido[2,3-b]pyrazinyl), pyrido[3,4-b]pyrazine (pyrido[3,4-b]pyrazinyl), pyrimido[5,4-d]pyrimidine (pyrimido[5,4-d]pyrimidinyl), pyrazino[2,3-b]pyrazine (pyrazino[2,3-b]pyrazinyl), or pyrimido[4,5-d]pyrimidine (pyrimido[4,5-d]pyrimidinyl).

“Amino” refers to a group —NH₂, which is unsubstituted. Where the amino is described as substituted or optionally substituted, the term includes groups of the form —NR^{x}R^{y}, where each of R^{x} and R^{y} is defined as further described herein.

The term “alkylamino” or “aminoalkyl” refer to a radical of the formula —NHR^{x} or —NR^{x}R^{y}, wherein each R^{x} and R^{y} is independently H, an alkyl group, or an alkylene group. For example, “alkylamino” can refer to a group —NR^{x}R^{y}, wherein one of R^{x} and R^{y} is an alkyl moiety and the other is H; and “dialkylamino” can refer to —NR^{x}R^{y}, wherein both of R^{x} and R^{y} are alkyl moieties, where the alkyl moieties have the specified number of carbon atoms (e.g., —NH(C₁-C₄ alkyl) or —N(C₁-C₄ alkyl)₂). In some embodiments, aminoalkyl refers to —NH-alkylene or alkylene-NH-alkylene, wherein each alkyklene is independent substituted or unsubstituted.

“Compounds” when used herein includes any pharmaceutically acceptable derivative or variation, including conformational isomers (e.g., cis and trans isomers), atropisomers (i.e., stereoisomers from hindered rotation), and all optical isomers (e.g., enantiomers and diastereomers), racemic, diastereomeric and other mixtures of such isomers, as well as solvates, hydrates, isomorphs, polymorphs, tautomers, esters, salt forms, and prodrugs. The expression “prodrug” refers to compounds that are drug precursors which following administration, release the drug in vivo via some chemical or physiological process (e.g., a prodrug on being brought to the physiological pH or through enzyme action is converted to the desired drug form). Exemplary prodrugs upon cleavage release the corresponding free acid, and such hydrolyzable ester-forming residues of the compounds of the disclosure include, but are not limited to, those having a carboxyl moiety wherein the free hydrogen is replaced by (C₁-C₄)alkyl, (C₂-C₇)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

If substituents are described as being “independently selected” from a group, each substituent is selected independent of the other. Each substituent therefore may be identical to or different from the other substituent(s).

“Optional” or “optionally” means that the subsequently described event or circumstance may, but need not occur, and the description includes instances where the event or circumstance occurs and instances in which it does not.

The terms “optionally substituted” and “substituted or unsubstituted” are used interchangeably to indicate that the particular group being described may have no non-hydrogen substituents (i.e., unsubstituted), or the group may have one or more non-hydrogen substituents (i.e., substituted). If not otherwise specified, the total number of substituents that may be present is equal to the number of H atoms present on the unsubstituted form of the group being described. Where an optional substituent is attached via a double bond, such as an oxo (═O) substituent, the group occupies two available valences, so the total number of other substituents that are included is reduced by two. In the case where optional substituents are selected independently from a list of alternatives, the selected groups may be the same or different. Throughout the disclosure, it will be understood that the number and nature of optional substituent groups will be limited to the extent that such substitutions make chemical sense to one of ordinary skill in the art. Examples of optional substituents include, but are not limited to, one or more of D, halogen, —CN, —NH₂, —NH(alkyl), —N(alkyl)₂, —OH, —CO₂H, —CO₂(alkyl), —C(═O)NH₂, —C(═O)NH(alkyl), —C(═O)N(alkyl)₂, —S(═O)₂NH₂, —S(═O)NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, arylsulfone, or oxo (═O).

As used herein, an arrowhead, “ [symbol] ” or wavy line, “ [symbol] ” denotes a point of attachment of a substituent to another group.

The term “preparation” in the EXAMPLES section below describe preparations of compounds that may be useful for synthesizing intermediates, which intermediates may be useful to one skilled in the art toward the synthesis of the protein degrader compounds as described herein.

The term “mammal” refers to human, livestock or companion animals.

The term “companion animal” or “companion animals” refers to animals kept as pets or household animals. Examples of companion animals include dogs, cats, and rodents including hamsters, guinea pigs, gerbils and the like, rabbits, ferrets.

The term “livestock” refers to animals reared or raised in an agricultural setting to make products such as food or fiber, or for its labor. In some embodiments, livestock are suitable for consumption by mammals, for example humans. Examples of livestock animals include cattle, goats, horses, pigs, sheep, including lambs, and rabbits.

“Patient” refers to warm blooded animals such as, for example, guinea pigs, mini pigs, mice, rats, gerbils, cats, rabbits, dogs, cattle, goats, sheep, horses, monkeys, chimpanzees, and humans.

The term “treating” or “treatment” means an alleviation of symptoms associated with a disease, disorder or condition, or halt of further progression or worsening of those symptoms.

Depending on the disease and condition of the patient, the term “treatment” as used herein may include one or more of curative, palliative and prophylactic treatment. Treatment can also include administering a pharmaceutical formulation in combination with other therapies.

“Therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “pharmaceutically acceptable” means the substance (e.g., the compounds of the invention) and any salt thereof, or composition containing the substance or salt of the invention that is suitable for administration to a patient. When referring to a compound of the disclosure (e.g., a compound of Formula I or Formula II), unless otherwise stated, it is understood that a pharmaceutically acceptable salt of said compound is also considered.

### Compounds of the Disclosure

In an embodiment of the compound, the compound has the Formula IA

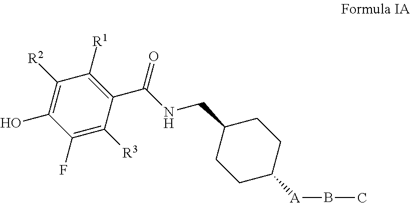

or a pharmaceutically acceptable salt of said compound.

In an embodiment of the compound, the compound has the Formula IB

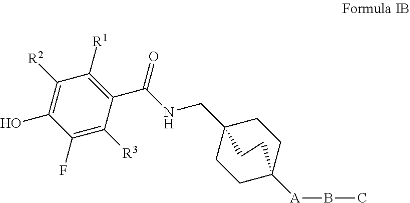

or a pharmaceutically acceptable salt of said compound.

In an embodiment of the compound, R² is F, or a pharmaceutically acceptable salt of said compound.

In an embodiment of the compound, A is thiazolyl, pyrazolyl, oxazolyl, imidazolyl, isoxazolyl, isothiazolyl, imidazotriazinyl, imidazopyridazinyl, imidazopyridinyl, benzoimidazolyl, benzothiazolyl, purinyl, pyridopyridazinyl, quinazolinyl, indazolyl, imidazopyridinyl, benzooxazolyl, pyrazolopyridinyl, isoindolinonyl, triazolyl, or oxadiazolyl, or a pharmaceutically acceptable salt of said compound.

In another embodiment of the compound, A is

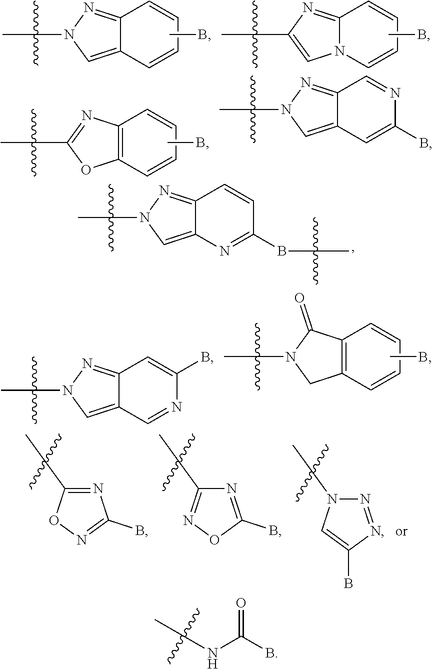

In another embodiment of the compound, B is absent or is H, pyridinyl, pyrimidinyl, pyridazinyl, pyrazinyl, pyrazolyl, piperazinyl, quinoxalinyl, phenyl, triazolyl, thiazolyl, thiadiazolyl, oxazolyl, imidazolyl, indazolyl, (C₁-C₆)alkyl, (C₁-C₆)fluoroalkyl, (C₁-C₆)alkoxy, bromo, chloro, fluoro, or oxo, and wherein B is optionally substituted with one or two fluoro, oxo, hydroxyl, (C₁-C₆)alkyl, (C₃-C₆)cycloalkyl, (C₁-C₆)fluoroalkyl, (C₁-C₆)alkoxy, or (C₃-C₆)cycloether; or a pharmaceutically acceptable salt of said compound.

In another embodiment of the compound, B is pyrimidinyl, (C₁-C₃)fluoroalkyl substituted pyrimidinyl, (C₁-C₃)alkyl substituted pyrazolyl, methoxy substituted pyridazinyl, difluoromethyl substituted pyrazinyl, trifluoromethyl substituted pyrimidinyl, or methoxy substituted pyrimidinyl; or a pharmaceutically acceptable salt of said compound.

In another embodiment of the compound, C is absent or is H, pyridinyl, piperazinyl, oxolanyl, (C₃-C₆)cycloalkyl, (C₁-C₆)alkyl, (C₁-C₆)fluoroalkyl, (C₁-C₆)alkoxy, cyano, bromo, chloro, fluoro, or oxo, and wherein C is optionally substituted with one, two or three fluoro, oxo, hydroxyl, (C₁-C₆)alkyl, (C₃-C₆)cycloalkyl, (C₁-C₆)fluoroalkyl, or (C₁-C₆)alkoxy; or a pharmaceutically acceptable salt of said compound.

In another embodiment of the compound, C is absent or is pyridinyl, piperazinyl, (C₃-C₆)cycloalkyl, (C₁-C₆)alkyl, (C₁-C₆)fluoroalkyl; and wherein C is optionally substituted with one, two or three fluoro, oxo, hydroxyl, or (C₁-C₆)alkyl; or a pharmaceutically acceptable salt of said compound. In an embodiment of the compound, the compound is 2,3,5-Trifluoro-4-hydroxy-N-[(4-{3-[5-(trifluoromethyl)pyrimidin-2-yl]-1,2,4-oxadiazol-5-yl}bicyclo[2.2.2]octan-1-yl)methyl]benzamide; 2,3,5-Trifluoro-4-hydroxy-N-({(1r,4r)-4-[6-(1-methyl-1H-pyrazol-4-yl)-2H-indazol-2-yl]cyclohexyl}methyl)benzamide; 2,3,5-Trifluoro-4-hydroxy-N-({4-[6-(pyrimidin-2-yl)-2H-indazol-2-yl]bicyclo[2.2.2]octan-1-yl}methyl)benzamide; 2,3,5-Trifluoro-4-hydroxy-N-({(1r,4r)-4-[6-(pyrimidin-5-yl)-2H-indazol-2-yl]cyclohexyl}methyl)benzamide; 2,3,5-Trifluoro-4-hydroxy-N-({4-[3-(6-methoxypyridazin-3-yl)-1,2,4-oxadiazol-5-yl]bicyclo[2.2.2]octan-1-yl}methyl)benzamide; N-[(4-{5-[5-(Difluoromethyl)pyrazin-2-yl]-1,2,4-oxadiazol-3-yl}bicyclo[2.2.2]octan-1-yl)methyl]-3,5-difluoro-4-hydroxybenzamide; 3,5-Difluoro-4-hydroxy-N-{[(1r,4r)-4-{3-[5-(trifluoromethyl)pyrimidin-2-yl]-1,2,4-oxadiazol-5-yl}cyclohexyl]methyl}benzamide; 3,5-Difluoro-4-hydroxy-N-({(1r,4r)-4-[6-(2-methoxypyrimidin-5-yl)-2H-pyrazolo[4,3-c]pyridin-2-yl]cyclohexyl}methyl)benzamide; 2,3,5-Trifluoro-4-hydroxy-N-[(4-{5-[2-(piperazin-1-yl)pyrimidin-4-yl]-1,2,4-oxadiazol-3-yl}bicyclo[2.2.2]octan-1-yl)methyl]benzamide or 2,3,5-Trifluoro-4-hydroxy-N-[(4-{5-[2-(4-methylpiperazin-1-yl)pyrimidin-4-yl]-1,2,4-oxadiazol-3-yl}bicyclo[2.2.2]octan-1-yl)methyl]benzamide, or a pharmaceutically acceptable salt of said compound.

In an embodiment of the compound, the compound is 2,3,5-trifluoro-4-hydroxy-N-[(4-{5-[2-(4-methylpiperazin-1-yl)pyrimidin-4-yl]-1,2,4-oxadiazol-3-yl}bicyclo[2.2.2]octan-1-yl)methyl]benzamide or a pharmaceutically acceptable salt of said compound.

### HSD17B13 Protein Degrader Compounds

Disclosed herein are protein degrader compounds comprising: 1) targeting protein ligand; 2) a linker of varying length and functionality; and 3) a ligand that interacts with the ubiquitin proteasome system (Degrons). The protein degrader compounds of the present disclosure are bifunctional and comprise a targeting ligand. The bifunctional protein degrader compounds of the disclosure can be used as therapeutics for treating various diseases, such as various liver diseases.

The protein degrader compounds of the present invention have the general structure: [Targeting ligand]-[Linker]-[Degron], wherein the linker is covalently bound to at least one degron and at least one targeting ligand. The degron is a compound capable of binding to a ubiquitin ligase, for example, an E3 ubiquitin ligase (e.g., cereblon (CRBN), von Hippel-Lindau (VHL), and the like). The targeting ligand is capable of binding to a targeted protein such as HSD17B13.

In an embodiment of the compound, the compound has the Formula II:

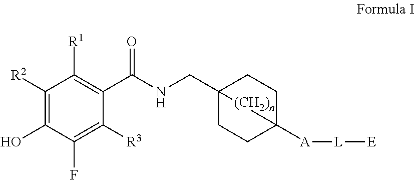

wherein:
A is —NH—C(O)—, —C(O)—, or heteroaryl, wherein heteroaryl has 1, 2, 3, or 4 heteroatoms selected from O, N, and S, and wherein A is optionally substituted with one or two R⁴;
R¹, R², and R³ are each independently selected from H and fluoro;
R⁴ is selected from oxo, hydroxyl, chloro, fluoro, (C₁-C₆)alkyl, (C₁-C₆)alkoxy, (C₁-C₆)fluoroalkyl, (C₃-C₆)cycloalkyl, and heterocyclyl, wherein the heterocyclyl has 1, 2, or 3 heteroatoms selected from O and N;
n is 0, 1, or 2;
L is a linker; and
E is an E3 ubiquitin ligase binder,
   or a pharmaceutically acceptable salt thereof.

In an embodiment of the protein degrader compound, the HSD17B13 targeting ligand segment of the compound has the Formula II-I:

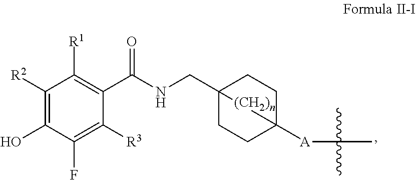

wherein:
A is —NH—C(O)—, —C(O)—, or heteroaryl, wherein heteroaryl has 1, 2, 3, or 4 heteroatoms selected from O, N, and S, and wherein A is optionally substituted with one or two R⁴;
R¹, R², and R³ are each independently selected from H and fluoro;
R⁴ is selected from oxo, hydroxyl, chloro, fluoro, (C₁-C₆)alkyl, (C₁-C₆)alkoxy, (C₁-C₆)fluoroalkyl, (C₃-C₆)cycloalkyl, and heterocyclyl, wherein the heterocyclyl has 1, 2, or 3 heteroatoms selected from O and N; and
n is 0, 1, or 2;
   or a pharmaceutically acceptable salt thereof.

In some embodiments, A is thiazolyl, pyrazolyl, oxazolyl, imidazolyl, isoxazolyl, isothiazolyl, imidazotriazinyl, imidazopyridazinyl, imidazopyridinyl, benzoimidazolyl, benzothiazolyl, purinyl, pyridopyridazinyl, quinazolinyl, indazolyl, imidazopyridinyl, benzooxazolyl, pyrazolopyridinyl, isoindolinonyl, triazolyl, or oxadiazolyl, or a pharmaceutically acceptable salt thereof.

In some embodiments, A is: In certain embodiments, the Linker is designed and optimized based on structure-activity relationship (SAR) and X-ray crystallography of the Targeting Ligand with regard to the location of attachment for the Linker. In certain embodiments, the optimal Linker length and composition vary by target and can be estimated based upon X-ray structures of the original Targeting Ligand bound to its target. Linker length and composition can be also modified to modulate metabolic stability and pharmacokinetic (PK) and pharmacodynamics (PD) parameters. In certain embodiments, where the Target Ligand binds multiple targets, selectivity may be achieved by varying Linker length where the ligand binds some of its targets in different binding pockets, e.g., deeper or shallower binding pockets than others.

The Linker (“L”) provides a covalent attachment between the Targeting Ligand and the Degron (i.e., E of Formula II). The Linker has two terminating groups, wherein one terminating group attaches to the Degron and the other terminating group attaches to the Targeting Ligand. The structure of the Linker may not be critical, provided it does not substantially interfere with the activity of the Targeting Ligand or the Degron.

In some embodiments, the Linker is C₂-C₂₀ alkylene or a polyethylene glycol (PEG) chain (e.g., CH₂CH₂-0 or (O—CH₂CH₂)). In other embodiments, the Linker may comprise and/or terminate (at one or both termini) by at least one of the following: —O—, —S—, —N(R^{L})—, —O═C—, —C(O)—, —C(O)O—, —OC(O)—, —OC(O)O—, —C(NOR^{L})—, —C(O)N(R^{L})—, —C(O)N(R^{L})C(O)—, —C(O)N(R^{L})C(O)N(R^{L})—, —N(R^{L})C(O)—, —N(R^{L})C(O)N(R^{L})—, —N(R^{L})C(O)O—, —OC(O)N(R^{L})—, —C(NR^{L})—, —N(R^{L})C(NR^{L})—, —C(NR^{L})N(R^{L})—, —N(R^{L})C(NR^{L})N(R^{L})—, —OB(CH₃)O—, —S(O)₂—, —OS(O)—, —S(O)O—, —S(O)—, —OS(O)₂—, —S(O)₂O—, —N(R^{L})S(O)₂—, —S(O)₂N(R^{L})—, —N(R^{L})S(O)—, —S(O)N(R^{L})—, —N(R^{L})S(O)₂N(R^{L})—, —N(R′)S(O)N(R′)—, C₃₋₁₂ carbocyclene, 3- to 12-membered heterocyclene, 5- to 12-membered heteroarylene, or arylene, or any combination thereof, wherein R^{L} is H or C₁-C₆ alkyl, wherein the interrupting and the one or both terminating groups may be the same or different.

In some embodiments, the Linker may be C₁-C₁₀ alkylene chain terminating with an NH-group, wherein the nitrogen is also bound to the Degron. In another embodiment, the Linker may be a C₁-C₁₀ alkylene chain or a PEG chain comprising 1-8 PEG units, wherein the Linker may comprise or terminating with —(CH₂)_{n′}—C(O)—NH—, where n′ is 0, 1, 2, 3, 4, or 5. For the Linker, “carbocyclene” refers to a bivalent carbocycle radical, which is optionally substituted. “Heterocyclylene” refers to a bivalent heterocyclyl radical which may be optionally substituted. “Heteroarylene” refers to a bivalent heteroaryl radical which may be optionally substituted. Nonlimiting examples of a Linker include —(CH₂)_{n′}—, —(CH₂CH₂—O)_{n′}—(CH₂)_{n′}—C(O)—, (CH₂)_{n′}—C(O)—N(R^{L})—(CH₂CH₂—O)_{n′}—(CH₂)_{n′}—C(O)—, —(CH₂CH₂—O)_{n′}—(CH₂)_{n′}—N(R^{L})—C(O)—, —(CH₂CH₂—O)_{n′}—(CH₂)_{n′}—C(O)—N(R^{L})—, —(CH₂)_{n′}-phenylene-N(R^{L})—C(O)—(CH₂)_{n′}—, —N(R^{L})—(CH₂)_{n′}—O-phenylene-(CH₂)_{n′}—N(R^{L})—(CH₂)_{n′}—, —(CH₂)_{n′}—C(O)—N(R^{L})-phenylene-C(O)—, —N(R^{L})—(CH₂)_{n′}-phenylene-(CH₂)_{n′}-heterocyclylene-, —(CH₂)_{n′}-phenylene-N(R^{L})—C(O)—(CH₂CH₂—O)_{n″}—, —(CH₂)_{n′}—, —(CH₂)_{n′}-phenylene-(CH₂)_{n″}-heterocyclylene-(CH₂)_{n′}—C(O)—N(R^{L})—(CH₂)_{n″}—, —(CH₂)_{n′}-phenylene-O—(CH₂)_{n′}-heterocyclylene-(CH₂)_{n′}—, —(CH₂)_{n′}-phenylene-(CH₂)_{n′}-heterocyclylene-(CH₂)_{n′}—O—, —(CH₂)_{n′}-heterocyclylene-(CH₂)_{n′} wherein R^{L} is H or C₁₋₆ alkyl; n′ is 0, 1, 2, 3, 4, 5, 6, 7, 8, 9, or 10; and n″ is 1, 2, 3, 4, 5, 6, 7, 8, 9, or 10.

In one embodiment of the protein degrader compound, the Linker has the Formula II-II:

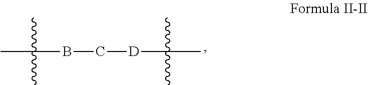

wherein:
B is absent; or aryl, heteroaryl, heterocyclyl, —C(O)—, (C₁-C₆)alkylene, (C₃-C₆)cycloalkylene, (C₁-C₆)fluoroalkylene, (C₁-C₆)alkoxy, or (C₁-C₆)fluoroalkoxy, wherein the heteroaryl, or heterocyclyl has 1, 2, or 3 heteroatoms selected from O, N, and S, and wherein B is optionally substituted with one or two R⁵;
C is absent; or —NH—C(O)—R⁷, —S(O)₂—R⁷, —O—S(O)₂—R⁷, —C(O)—, (C₁-C₆)alkylene, (C₁-C₆)aminoalkylene, (C₃-C₆)cycloalkylene, (C₁-C₆)alkoxy, (C₃-C₆)cycloether, (C₁-C₆)fluoroalkylene, (C₁-C₆)fluoroalkoxy, aryl, heteroaryl, or heterocyclyl, wherein the heteroaryl or heterocyclyl has 1, 2, or 3 heteroatoms selected from O, N, and S, and wherein C is optionally substituted with one, two or three R⁶;
D is (C₁-C₆)alkylene, (C₁-C₆)aminoalkylene, —NH(C₁-C₆)alkylene, (C₁-C₆)alkoxy, —C(O)—, aryl, heteroaryl, heterocyclyl, (C₀-C₆)alkylene-heterocyclyl-C(O)—, —C(O)—(C₁-C₆)alkylene, heterocyclyl-(C₁-C₆)alkylene-aryl-(C₁-C₆)alkoxy, (C₁-C₆)heterocyclyl-(C₁-C₆)heterocyclyl-C(O)—, (C₀-C₂)alkylene-aryl-(C₁-C₆)alkoxy, —O-heterocyclyl-C(O)—, (C₁-C₆)cycloalkyl-(C₁-C₆)heterocyclyl, wherein the heteroaryl or heterocyclyl has 1, 2, or 3 heteroatoms selected from O, N, and S, wherein D is optionally substituted with one or two R⁸; or a bond;
R⁵, R⁶, and R⁸ are each independently selected from oxo, hydroxyl, halogen, (C₁-C₆)alkyl, (C₁-C₆)alkoxy, (C₁-C₆)fluoroalkyl, (C₃-C₆)cycloalkyl, heteroaryl, and heterocyclyl, wherein the heterocyclyl has 1, 2, or 3 heteroatoms selected from O and N;
R⁷ is (C₁-C₆)alkyl, (C₁-C₆)alkoxy, (C₁-C₆)fluoroalkyl, or (C₃-C₆)cycloalkyl;
   or a pharmaceutically acceptable salt thereof.

In some embodiments, B is pyridinyl, pyrimidinyl, pyridazinyl, pyrazinyl, pyrazolyl, piperazinyl, quinoxalinyl, phenyl, triazolyl, thiazolyl, thiadiazolyl, oxazolyl, imidazolyl, indazolyl, (C₁-C₆)alkylene, (C₁-C₆)fluoroalkylene, (C₁-C₆)alkoxy, pyrrolopyridinyl, isoindolinyl, isoquinolinyl, tetrahydroisoquinolinyl, thiazolopyridinyl, tetrahydrothiazolopyridinyl, imidazopyrazinyl, tetrahydroimidazopyrazinyl, pyrazolopyrazinyl, tetrahydropyrazolopyrazinyl, phenyl, spiro[4.5]decanyl, spiro[3.4]octanyl, or spiro[4.5]decan-1-onyl, wherein B is optionally substituted with one or two halogen, oxo, hydroxyl, (C₁-C₆)alkyl, (C₃-C₆)cycloalkyl, (C₁-C₆)fluoroalkyl, (C₁-C₆)alkoxy, or (C₃-C₆)cycloether. In some embodiments, B is (C₁-C₆)alkylene, (C₁-C₆)heteroalkylene, (C₁-C₆)alkoxy, phenyl, isoindolinyl, pyrimidinyl, pyridazinyl, pyrazolyl, 6,7-dihydro-5H-pyrrolo[3,4-b]pyridinyl, tetrahydroisoquinolinyl, tetrahydrothiazolo[5,4-c]pyridinyl, tetrahydroimidazo[1,2-a]pyrazinyl, 6-oxa-2,9-diazaspiro[4.5]decanyl, 2,6-diazaspiro[3.4]octanyl, 7-diazaspiro[4.5]decan-1-onyl, or tetrahydropyrazolo[1,5-a]pyrazinyl. In some embodiments, B is:

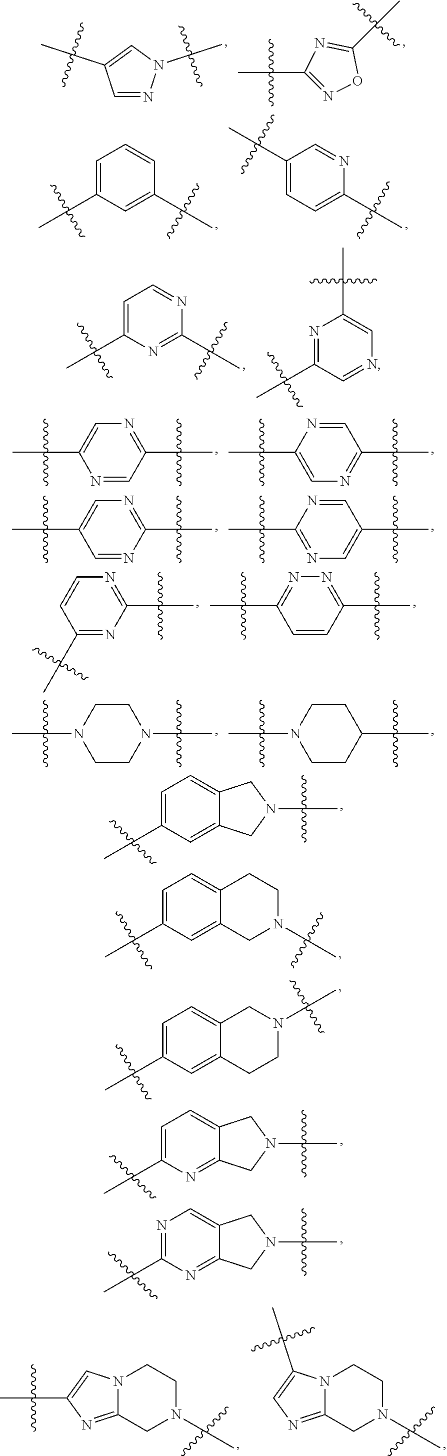

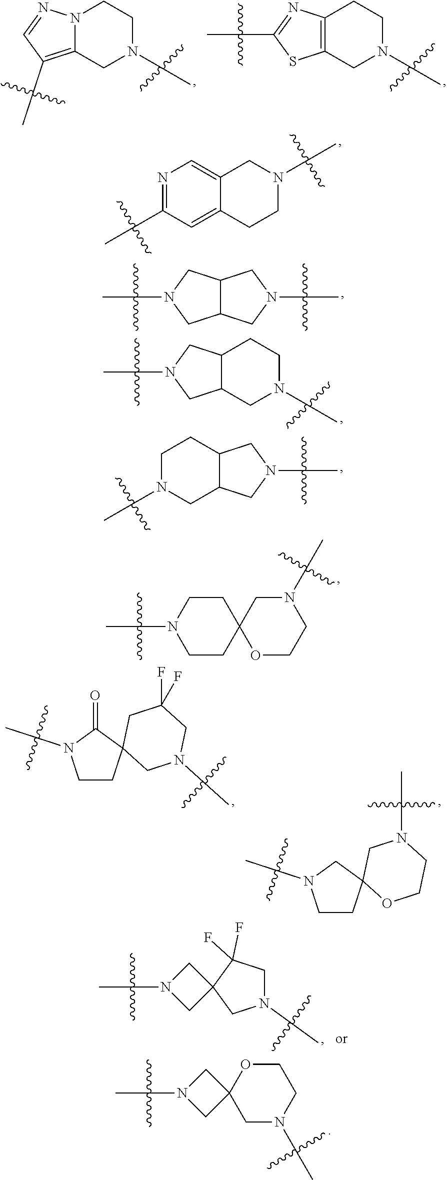

In some embodiments, B is absent. In some embodiments, B is (C₁-C₃)alkylene. In some embodiments, B is

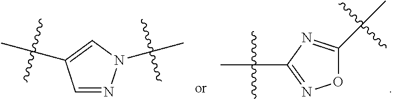 [SMILES: *c1cnn(*)c1 ; *c1noc(*)n1]

In some embodiments, B is,

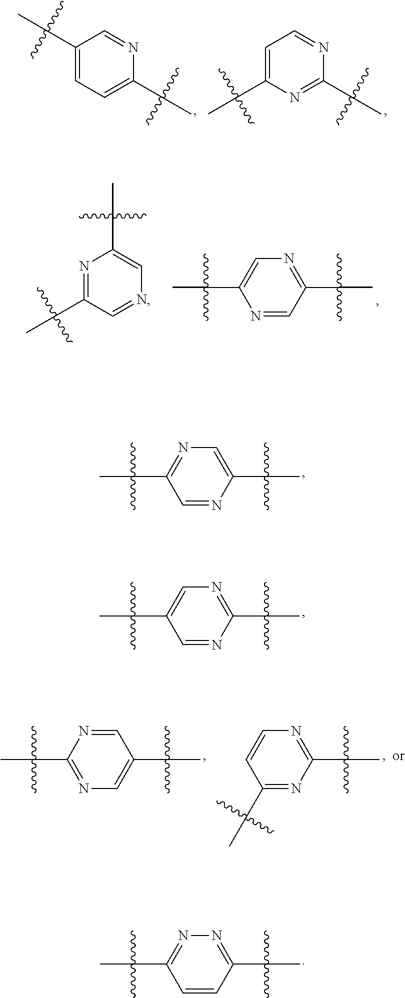

In some embodiments, B is

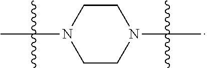 [SMILES: *N1CCN(*)CC1]

In some embodiments, B is

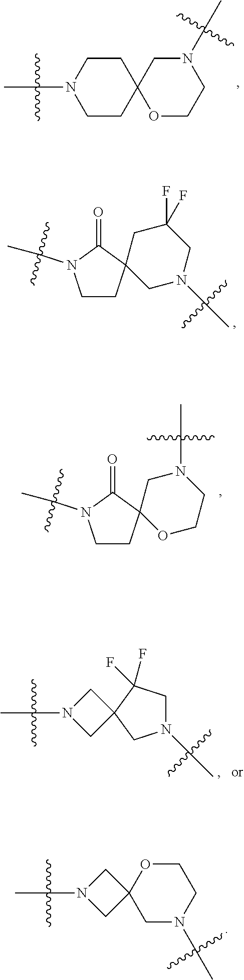 [SMILES: *N1CCC2(CC1)CN(*)CCO2 ; *N1CC(F)(F)CC2(CCN(*)C2=O)C1 ; *N1CCOC2(CCN(*)C2=O)C1 ; *N1CC(F)(F)C2(C1)CN(*)C2 ; *N1CCOC2(C1)CN(*)C2]

In some embodiments, C is (C₁-C₃)alkylene, (C₁-C₆)aminoalkylene, (C₁-C₆)alkoxy, pyridinyl, oxolanyl, (C₃-C₆)cycloalkyl, (C₁-C₆)fluoroalkylene, —C(O)—, piperazinyl, piperidinyl, azetidinyl, azaspiroundecanyl, azaspirononanyl, azaspiroundecanyl, diazaspirooctanyl, diazaspirodecanyl, diazaspirononanyl, diazaspirododecanyl, diazaspiroundecanyl, oxadiazaspirononanyl, oxadiazaspiroundecanyl, oxa-azaspirodecanyl, decahydronaphthyridinyl, octahydropyrrolopyridinyl, or octahydropyridopyrazinyl; wherein C is optionally substituted with one, two or three halogen, oxo, hydroxyl, (C₁-C₆)alkyl, (C₃-C₆)cycloalkyl, (C₁-C₆)fluoroalkyl, or (C₁-C₆)alkoxy. In some embodiments, C is (C₁-C₆)alkylene, (C₁-C₆)aminoalkylene, (C₁-C₆)alkoxy, piperazinyl, piperidinyl, azetidinyl, —C(O)—, 5-oxa-diazaspiro[3.5]nonanyl, 1-oxa-diazaspiro[5.5]undecanyl, 3-azaspiro[5.5]undecanyl, 1-oxa-8-azaspiro[4.5]decanyl, 8-azaspiro[4.5]decanyl, 7-azaspiro[3.5]nonanyl, 2,8-diazaspiro[4.5]decanyl, 1-oxa-4,9-diazaspiro[5.5]undecanyl, 3,9-diazaspiro[5.5]undecanyl, 1-oxa-8-azaspiro[4.5]decanyl, 3-azaspiro[5.5]undecanyl, 2-azaspiro[5.5]undecanyl, 2,6-diazaspiro[3.4]octanyl, 3,9-diazaspiro[5.6]dodecanyl, 2,7-diazaspiro[3.5]nonanyl, 2,9-diazaspiro[5.5]undecanyl, decahydro-1,5-naphthyridinyl, octahydro-1H-pyrrolo[3,4-c]pyridinyl, 2,6-diazaspiro[3.5]nonanyl, 2-azaspiro[3.5]nonanyl, octahydro-1H-pyrrolo[3,2-c]pyridinyl, octahydro-2H-pyrido[1,2-a]pyrazinyl.

In some embodiments, C is:

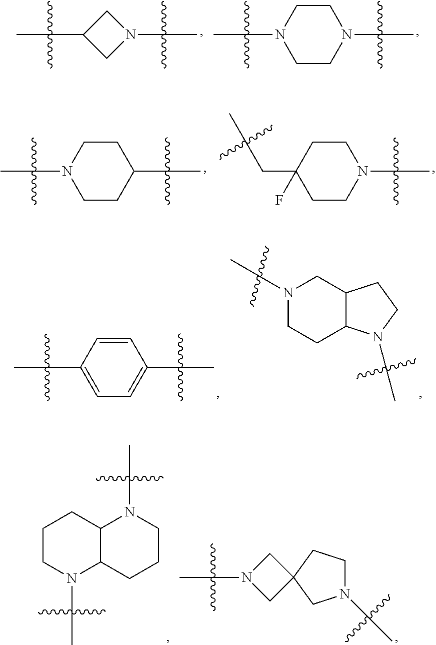

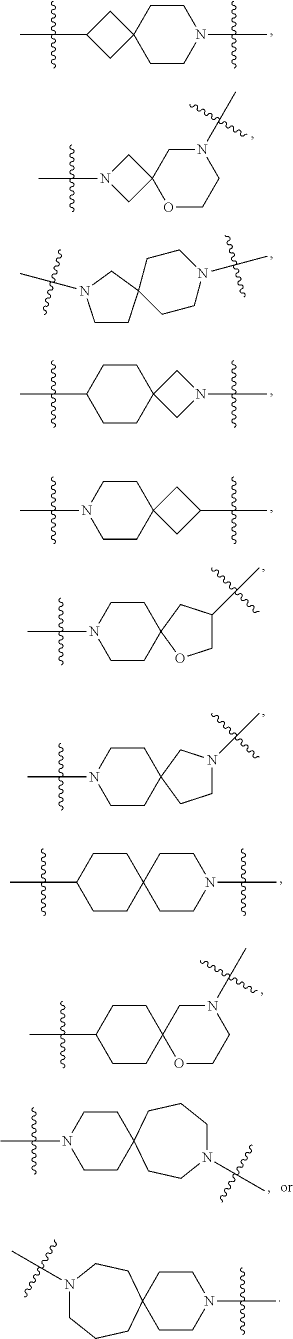

In some embodiments, C is absent. In some embodiments, C is (C₁-C₆)alkylene, (C₁-C₆)aminoalkylene, or (C₁-C₆)alkoxy. In some embodiments, C is

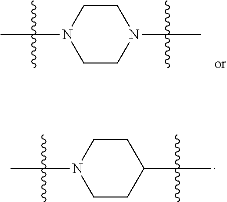 [SMILES: *N1CCN(*)CC1 ; *C1CCN(*)CC1]

In some embodiments, C is

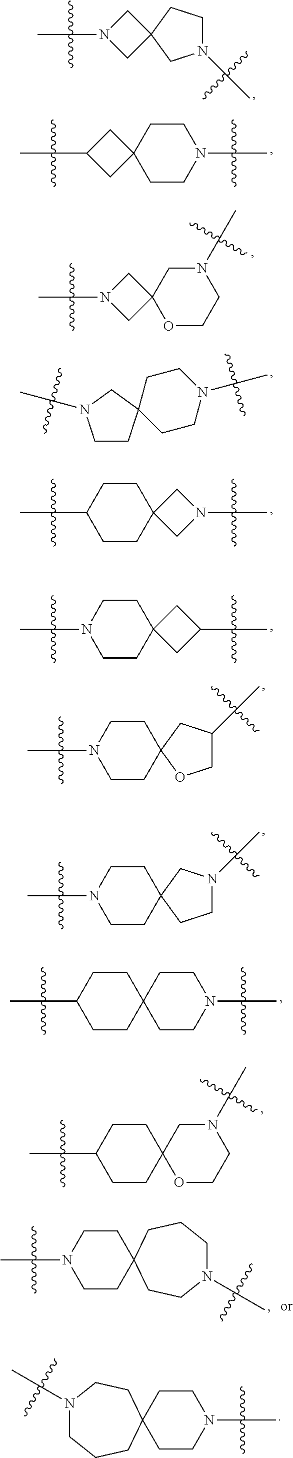

In some embodiments, D is a bond. In some embodiments, D is (C₁-C₆)alkylene, (C₁-C₆)aminoalkylene, —NH(C₁-C₆)alkylene, (C₁-C₆)alkoxy, —C(O)—, or —C(O)—(C₁-C₆)alkylene. In some embodiments, D is methylene, ethylene, or propylene. In some embodiments, D is (C₀-C₆)alkylene-heterocyclyl-C(O)—, heterocyclyl-(C₁-C₆)alkylene-aryl-(C₁-C₆)alkoxy, (C₁-C₆)heterocyclyl-(C₁-C₆)heterocyclyl-C(O)—, (C₀-C₂)alkylene-aryl-(C₁-C₆)alkoxy, —O— heterocyclyl-C(O)—, (C₁-C₆)cycloalkyl-(C₁-C₆)heterocyclyl.

In some embodiments, D is methylene, ethylene, or propylene. In some embodiments, D is heterocyclyl-C(O)—. In some embodiments, D is —C(O)—(C₁-C₆)alkylene. In some embodiments, D is —C(O)—. In some embodiments, D is

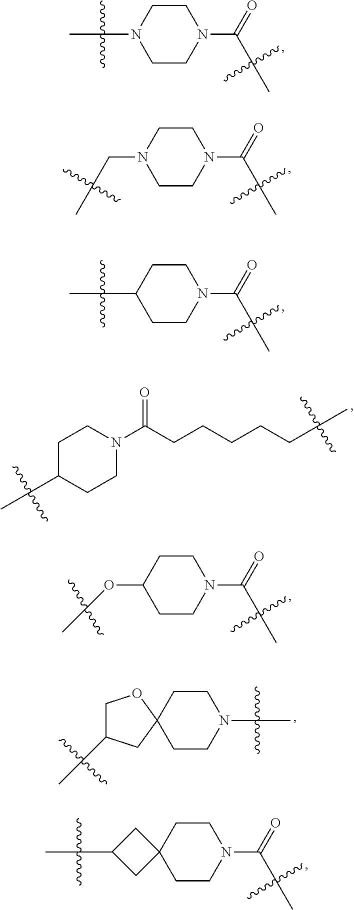

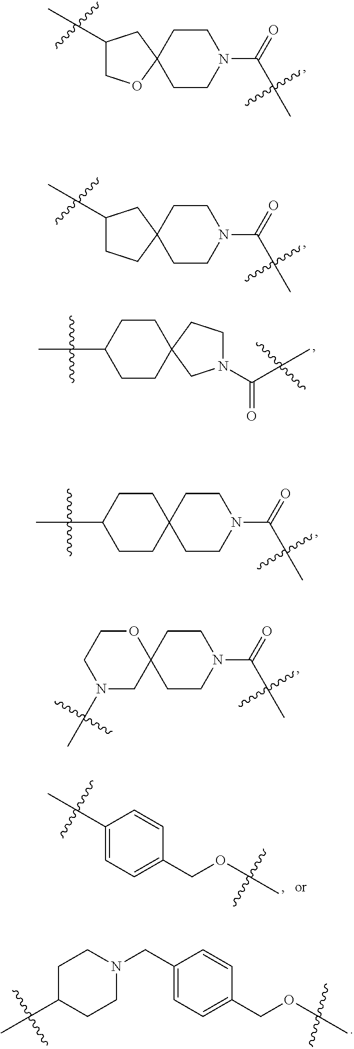

In some embodiments, A is heteroaryl, B is heteroaryl, C is absent, and D is (C₁-C₆)alkylene. In some embodiments, A is heteroaryl, B is heteroaryl, C is heterocyclyl, and D is (C₁-C₃)alkylene. In some embodiments, A is indazolyl, oxadiazolyl, thiazolyl; B is pyridazinyl, pyrazinyl, pyrimidinyl, piperazinyl, pyrazolyl, isoindolinyl, or dihydropyrrolopyridinyl; C is absent, (C₁-C₃)alkylene, (C₁-C₃)alkoxy, or piperidinyl; and D is methylene, ethylene, or propylene. In some embodiments, A is indazolyl or oxadiazolyl; B is pyrimidinyl; C is piperazinyl; D is methylene, ethylene, or propylene. In some embodiments, A is indazolyl; B is pyrimidinyl; C is (C₁-C₃)alkoxy; and D is heterocyclyl-C(O)—. In some embodiments, A is oxadiazolyl, B is pyrimidinyl, C is piperazinyl, and D is propylene.

A degron (i.e., “E” of Formula II) is small in size and highly effective in recruiting targeted proteins for degradation. The degron links a targeted protein to a ubiquitin ligase for proteasomal degradation via a linker and targeting ligand. In certain embodiments, the Degron is a compound that can bind to a ubiquitin ligase. In further embodiments, the Degron is a compound that can bind to a E3 Ubiquitin Ligase (e.g., cereblon), and the Degron can be a thalidomide, lenalidomide, pomalidomide, or iberdomide, or newer IMiDs CRBN ligands disclosed in WO2019/060693, WO2019/140387, WO2019/236483 or analogues thereof. In further embodiments, the Degron can bind to a E3 Ubiquitin Ligase, such as von Hippel-Lindau ligand. See, e.g., WO2020/092907; WO2013106643; Buckley et al. J. Am. Chem. Soc. 2012, 134, 4465-4468, “Targeting the Von Hippel-Lindau E3 Ubiquitin Ligase Using Small Molecules to Disrupt the VHL/Hif-1alpha Interaction”, Soares et al. J. Med. Chem. 2019, 61, 599-618, “Group-Based Optimization of Potent and Cell-Active Inhibitors of the von Hippel-Lindau (VHL) E3 Ubiquitin Ligase: Structure-Activity Relationships Leading to the Chemical Probe (2S,4R)-1-((S)-2-(1-Cyanocyclopropanecarboxamido)-3,3-dimethylbutanoyl)-4-hydroxy-N-(4-(4-methylthiazol-5-yl)benzyl)pyrrolidine-2-carboxamide (VH298)”. In further embodiments, the Degron can bind a E3 Ubiquitin Ligase, including inhibitors of apoptosis protein ligases (IAP1, IAP2, XIAP). See, e.g., Itoh et al, J. Am. Chem. Soc. 2010, 132, 5820-5826 “Protein Knockdown Using Methyl Bestatin-Ligand Hybrid Molecules: Design and Synthesis of Inducers of Ubiquitination-Mediated Degradation of Cellular Retinoic Acid-Binding Proteins’, Mares et al. Commun. Biol. 2020, 3, 140, “Extended pharmacodynamic responses observed upon PROTAC-mediated degradation of RIPK2′, and Tinworth et al. ACS Chem. Biol. 2019, 14, 342-347, “PROTAC-Mediated Degradation of Bruton's Tyrosine Kinase Is Inhibited by Covalent Binding.’ In further embodiments, the Degron can bind ubiquitin proteasome proteins that can induce degradation including, but not limited to, the Hsp70/90 chaperone complex (WO2020/207395), Usp14 (WO2019/238886), UchL5 (WO2019238816), BILO (WO201719705), and Rpn11 (WO2019/238817).

In some embodiments, E comprises a benzimidazolinone, a dihydropyrimidine-dione, or a thalidomide.

In some embodiments, the degron (i.e., “E” for Formula II) has the Formula II-IIIaa or II-IIIab:

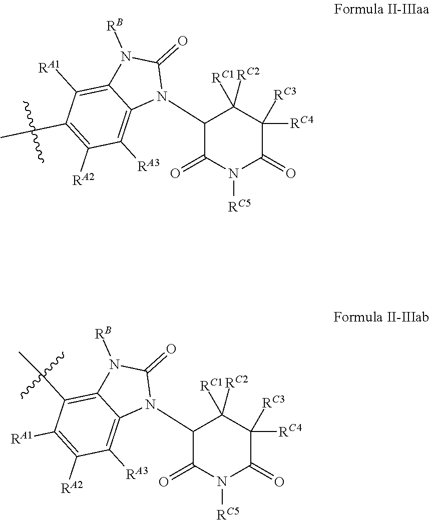

wherein: R^{A1}, R^{A2}, and R^{A3} are each independently H, hydroxyl, halogen, (C₁-C₆)alkyl, (C₁-C₆)heteroalkyl, (C₁-C₆)alkoxy, or NH₂; R^{B} is H or (C₁-C₆)alkyl; and R^{C1}, R^{C2}, R^{C3}, and R^{C4} are each independently H, hydroxyl, halogen, (C₁-C₆)alkyl, (C₁-C₆)heteroalkyl, (C₁-C₆)alkoxy, or NH₂. In some embodiments, R^{C5} is H, (C₁-C₆)alkyl, (C₁-C₆)heteroalkyl, (C₁-C₆)alkoxy, or NH₂. In some embodiments, R^{A1}, R^{A2}, and R^{A3} are each independently H; R^{B} is (C₁-C₃)alkyl; and R^{C1}, R^{C2}, R^{C3}, R^{C4} are each independently H; and R^{C5} is H. In some embodiments, E is

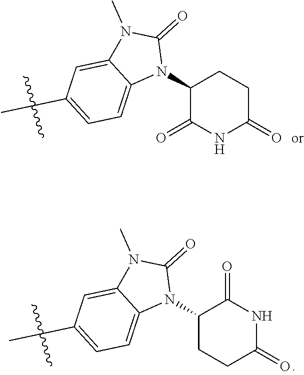 [SMILES: *c1ccc2c(c1)n(C)c(=O)n2[C@H]1CCC(=O)NC1=O ; *c1ccc2c(c1)n(C)c(=O)n2[C@H]1CCC(=O)NC1=O]

In some embodiments, E is

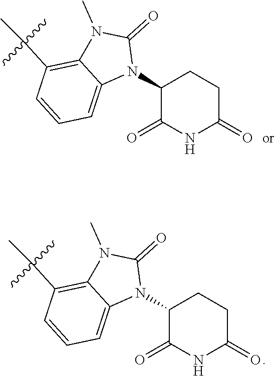 [SMILES: *c1cccc2c1n(C)c(=O)n2[C@H]1CCC(=O)NC1=O ; *c1cccc2c1n(C)c(=O)n2[C@@H]1CCC(=O)NC1=O]

In some embodiments, the degron (i.e., “E” for Formula II) has the Formula II-IIIb:

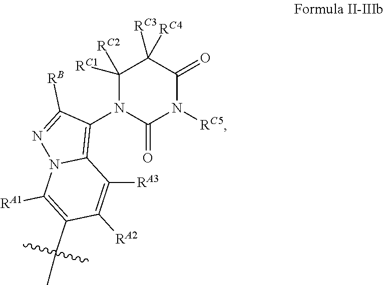

wherein: R^{A1}, R^{A2}, and R^{A3} are each independently H, hydroxyl, halogen, (C₁-C₆)alkyl, (C₁-C₆)heteroalkyl, (C₁-C₆)alkoxy, or NH₂; R^{B} is H or (C₁-C₆)alkyl; and R^{C1}, R^{C2}, R^{C3}, R^{C4}, and R^{C5} are each independently H, hydroxyl, halogen, or (C₁-C₆)alkyl, (C₁-C₆)heteroalkyl, (C₁-C₆)alkoxy, or NH₂. In some embodiments, E is

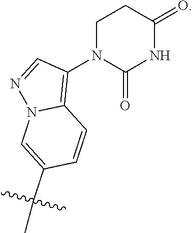 [SMILES: *c1ccc2c(N3CCC(=O)NC3=O)cnn2c1]

In some embodiments, the degron (i.e., “E” for Formula II) has the Formula II-IIIc:

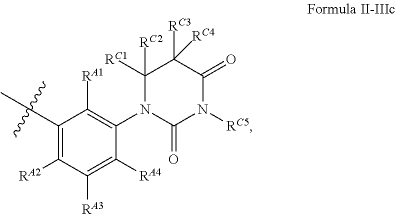

wherein: R^{A1}, R^{A2}, R^{A3} and R^{A4} are each independently H, hydroxyl, halogen, (C₁-C₆)alkyl, (C₁-C₆)heteroalkyl, (C₁-C₆)alkoxy, or NH₂; and R^{C1}, R^{C2}, R^{C3}, and R^{C4} are each independently H, hydroxyl, halogen, (C₁-C₆)alkyl, (C₁-C₆)heteroalkyl, (C₁-C₆)alkoxy, or NH₂. In some embodiments, R^{C5} is H, (C₁-C₆)alkyl, (C₁-C₆)heteroalkyl, (C₁-C₆)alkoxy, or NH₂. In some embodiments, R^{A1}, R^{A2}, and R^{A3} are each independently H; R^{A4} is (C₁-C₃)alkyl or halogen; and R^{C1}, R^{C2}, R^{C3}, R^{C4}, and R^{C5} are each independently H. In some embodiments, E is

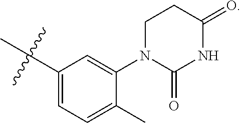 [SMILES: *c1ccc(C)c(N2CCC(=O)NC2=O)c1]

In some embodiments, the degron (i.e., “E” for Formula II) has the Formula II-IIId:

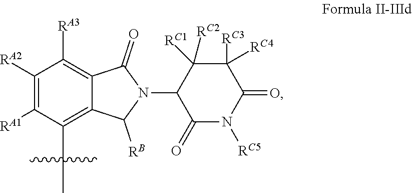

wherein: R^{A1}, R^{A2}, and R^{A3} are each independently H, hydroxyl, halogen, (C₁-C₆)alkyl, (C₁-C₆)heteroalkyl, (C₁-C₆)alkoxy, or NH₂; R^{B} is H or (C₁-C₆)alkyl; and R^{C1}, R^{C2}, R^{C3}, and R^{C4} are each independently H, hydroxyl, halogen, (C₁-C₆)alkyl, (C₁-C₆)heteroalkyl, (C₁-C₆)alkoxy, or NH₂. In some embodiments, R^{C5} is H, (C₁-C₆)alkyl, (C₁-C₆)heteroalkyl, (C₁-C₆)alkoxy, or NH₂. In some embodiments, R^{A1}, R^{A2}, and R^{A3} are each independently H; R^{B} is H; and R^{C1}, R^{C2}, R^{C3}, R^{C4}, and R^{C5} are each independently H. In some embodiments, E is

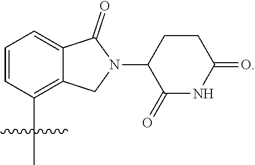 [SMILES: *c1cccc2c1CN(C1CCC(=O)NC1=O)C2=O]

In some embodiments, the degron (i.e., “E” for Formula II) has the Formula II-IIIe:

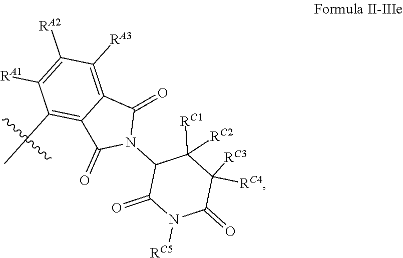

wherein: R^{A1}, R^{A2}, and R^{A3} are each independently H, hydroxyl, halogen, (C₁-C₆)alkyl, (C₁-C₆)heteroalkyl, (C₁-C₆)alkoxy, or NH₂; R^{C1}, R^{C2}, R^{C3}, and R^{C4} are each independently H, hydroxyl, halogen, (C₁-C₆)alkyl, (C₁-C₆)heteroalkyl, (C₁-C₆)alkoxy, or NH₂. In some embodiments, R^{C5} is H, (C₁-C₆)alkyl, (C₁-C₆)heteroalkyl, (C₁-C₆)alkoxy, or NH₂. In some embodiments, E is

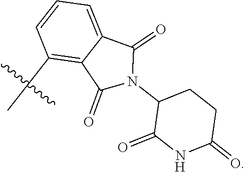

In some embodiments, the compound has the Formula IIA:

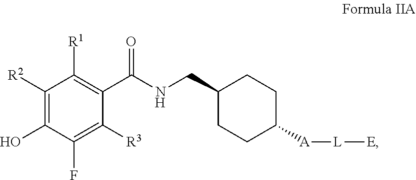

or a pharmaceutically acceptable salt thereof.

In some embodiments, the compound has the Formula IIB:

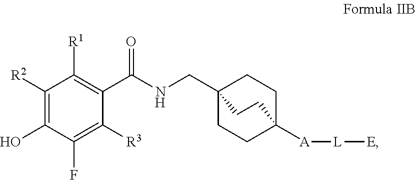

or a pharmaceutically acceptable salt thereof.

In some embodiments, the compound is selected from the group consisting of:
N-{[(1r,4r)-4-{6-[2-(3-{1-[(3RS)-2,6-dioxopiperidin-3-yl]-3-methyl-2-oxo-2,3-dihydro-1H-benzimidazol-5-yl}propyl)-2,3-dihydro-1H-isoindol-5-yl]-2H-indazol-2-yl}cyclohexyl]methyl}-2,3,5-trifluoro-4-hydroxybenzamide;
N-{[4-(7-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-5-yl}imidazo[1,2-a]pyridin-2-yl)bicyclo[2.2.2]octan-1-yl]methyl}-2,3,5-trifluoro-4-hydroxybenzamide;
N-{[(1r,4r)-4-(6-{2-[8-(3-{1-[(3RS)-2,6-dioxopiperidin-3-yl]-3-methyl-2-oxo-2,3-dihydro-1H-benzimidazol-5-yl}propyl)-5-oxa-2,8-diazaspiro[3.5]nonan-2-yl]pyrimidin-5-yl}-2H-indazol-2-yl)cyclohexyl]methyl}-2,3,5-trifluoro-4-hydroxybenzamide;
N-{[(1r,4r)-4-{6-[6-(3-{1-[(3RS)-2,6-dioxopiperidin-3-yl]-3-methyl-2-oxo-2,3-dihydro-1H-benzimidazol-5-yl}propyl)-6,7-dihydro-5H-pyrrolo[3,4-b]pyridin-2-yl]-2H-indazol-2-yl}cyclohexyl]methyl}-2,3,5-trifluoro-4-hydroxybenzamide;
N-{[4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)bicyclo[2.2.2]octan-1-yl]methyl}-2,3,5-trifluoro-4-hydroxybenzamide;
N-{[(1r,4r)-4-(6-{5-[4-(2-{1-[(3RS)-2,6-dioxopiperidin-3-yl]-3-methyl-2-oxo-2,3-dihydro-1H-benzimidazol-5-yl}ethyl)piperazin-1-yl]pyrazin-2-yl}-2H-indazol-2-yl)cyclohexyl]methyl}-2,3,5-trifluoro-4-hydroxybenzamide;
N-{[(1r,4r)-4-(6-{6-[4-(2-{1-[(3RS)-2,6-dioxopiperidin-3-yl]-3-methyl-2-oxo-2,3-dihydro-1H-benzimidazol-5-yl}ethyl)piperazin-1-yl]pyrazin-2-yl}-2H-indazol-2-yl)cyclohexyl]methyl}-2,3,5-trifluoro-4-hydroxybenzamide;
N-{[(1r,4r)-4-{6-[4-(2-{1-[(3RS)-2,6-dioxopiperidin-3-yl]-3-methyl-2-oxo-2,3-dihydro-1H-benzimidazol-5-yl}ethyl)piperazin-1-yl]-2H-indazol-2-yl}cyclohexyl]methyl}-2,3,5-trifluoro-4-hydroxybenzamide;
N-{[(1r,4r)-4-{6-[5-(4-{3-[3-(2,4-dioxo-1,3-diazinan-1-yl)pyrazolo[1,5-a]pyridin-6-yl]propyl}piperazin-1-yl)pyrazin-2-yl]-2H-indazol-2-yl}cyclohexyl]methyl}-2,3,5-trifluoro-4-hydroxybenzamide, hydrochloride salt;
N-{[4-(4-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,3-thiazol-2-yl)bicyclo[2.2.2]octan-1-yl]methyl}-2,3,5-trifluoro-4-hydroxybenzamide;
N-{[4-(3-{6-[4-(3-{1-[(3RS)-2,6-dioxopiperidin-3-yl]-3-methyl-2-oxo-2,3-dihydro-1H-benzimidazol-5-yl}propyl)piperazin-1-yl]pyridazin-3-yl}-1,2,4-oxadiazol-5-yl)bicyclo[2.2.2]octan-1-yl]methyl}-2,3,5-trifluoro-4-hydroxybenzamide;
N-{[4-(2-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,3-thiazol-4-yl)bicyclo[2.2.2]octan-1-yl]methyl}-2,3,5-trifluoro-4-hydroxybenzamide;
N-{[(1r,4r)-4-{5-[4-(3-{1-[(3RS)-2,6-dioxopiperidin-3-yl]-3-methyl-2-oxo-2,3-dihydro-1H-benzimidazol-5-yl}propyl)piperazin-1-yl]-2H-pyrazolo[4,3-b]pyridin-2-yl}cyclohexyl]methyl}-2,3,5-trifluoro-4-hydroxybenzamide;
N-{[(1r,4r)-4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)cyclohexyl]methyl}-2,3,5-trifluoro-4-hydroxybenzamide;
N-{[4-(2-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,3-oxazol-5-yl)bicyclo[2.2.2]octan-1-yl]methyl}-2,3,5-trifluoro-4-hydroxybenzamide;
N-{[(1r,4r)-4-{6-[2-(4-{3-[3-(2,4-dioxo-1,3-diazinan-1-yl)imidazo[1,2-a]pyridin-7-yl]propyl}piperazin-1-yl)pyrimidin-5-yl]-2H-indazol-2-yl}cyclohexyl]methyl}-2,3,5-trifluoro-4-hydroxybenzamide;
N-{[(1r,4r)-4-{6-[2-(4-{8-[3-(2,4-dioxo-1,3-diazinan-1-yl)-4-methylbenzoyl]-1-oxa-8-azaspiro[4.5]decan-3-yl}piperazin-1-yl)pyrimidin-5-yl]-2H-indazol-2-yl}cyclohexyl]methyl}-2,3,5-trifluoro-4-hydroxybenzamide;
N-{[(1r,4r)-4-{6-[4-({4-[({2-[(3RS)-2,6-dioxopiperidin-3-yl]-1-oxo-2,3-dihydro-1H-isoindol-4-yl}oxy)methyl]phenyl}methyl)piperazin-1-yl]-2H-indazol-2-yl}cyclohexyl]methyl}-3,5-difluoro-4-hydroxybenzamide; and
N-{[(1r,4r)-4-{6-[2-(2-{4-[3-(2,4-dioxo-1,3-diazinan-1-yl)-4-methylbenzoyl]piperazin-1-yl}ethoxy)pyrimidin-5-yl]-2H-indazol-2-yl}cyclohexyl]methyl}-2,3,5-trifluoro-4-hydroxybenzamide,

or a pharmaceutically acceptable salt thereof.

In one embodiment, the compound has the structure:

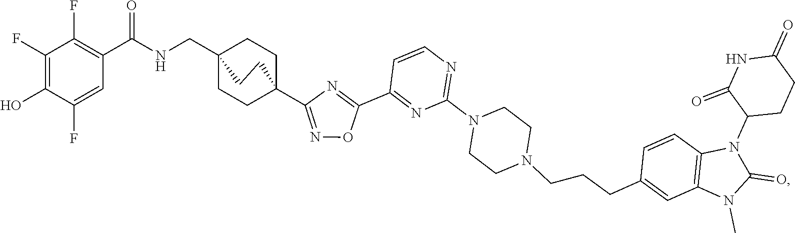

or a pharmaceutically acceptable salt thereof. In one embodiment, the compound is a pharmaceutically acceptable salt of N-{[4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)bicyclo[2.2.2]octan-1-yl]methyl}-2,3,5-trifluoro-4-hydroxybenzamide. In one embodiment, the compound is N-{[4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)bicyclo[2.2.2]octan-1-yl]methyl}-2,3,5-trifluoro-4-hydroxybenzamide, hydrochloride salt. In some embodiments, the compound is N-{[4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)bicyclo[2.2.2]octan-1-yl]methyl}-2,3,5-trifluoro-4-hydroxybenzamide. Another embodiment includes a compound selected from any of the Examples described herein, or a pharmaceutically acceptable salt thereof.

Another embodiment includes a prodrug of any of the Examples described herein, or a pharmaceutically acceptable salt thereof.

Another embodiment includes a phosphate ester prodrug of any of the Examples described herein, or a pharmaceutically acceptable salt thereof.

Another embodiment includes any novel genus of intermediates described in the General Schemes or Examples.

Another embodiment includes any novel specific compounds described in the Preparations and/or compounds or intermediates described in the Examples as described herein.

Another embodiment includes any novel process described herein.

All pharmaceutically acceptable isotopically-labelled compounds of Formula I or Formula II are within scope of this application wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, nitrogen, such as ¹³N and ¹⁵N, oxygen, such as ¹⁵O, ¹⁷O and ¹⁸O, and sulphur, such as ³⁵S Certain isotopically-labelled compounds of Formula I or Formula II for example, those incorporating a radioactive isotope, are useful in drug and/or substrate tissue distribution studies.

The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., 2H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Tomography (PET) studies for examining substrate receptor occupancy.

Isotopically-labelled compounds of Formula I or Formula II can generally be prepared by conventional techniques known to those skilled in the art or by processes analogous to those described in the accompanying Examples and Preparations using an appropriate isotopically-labelled reagent in place of the non-labelled reagent previously employed.

Certain compounds of Formula I or Formula II and intermediates described herein may exist in more than one crystal form (generally referred to as “polymorphs”). Polymorphs may be prepared by crystallization under various conditions, for example, using different solvents or different sol-vent mixtures for recrystallization; crystallization at different temperatures; and/or various modes of cooling, ranging from very fast to very slow cooling during crystallization. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffraction or such other techniques.

Salts encompassed within the term “pharmaceutically acceptable salts” refer to the compounds of this invention which are generally prepared by reacting the free base or free acid with a suitable organic or inorganic acid, or a suitable organic or inorganic base, respectively, to provide a salt of the compound of the invention that is suitable for administration to a patient. Base salts are preferred, however, some compounds may also form acid salts. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, calcium, choline, diethylamine, glycine, lysine, magnesium, meglumine, olamine, potassium, sodium, trimethamine and zinc salts. Hemisalts of acids and bases may also be formed, for example, hemisulfate and hemicalcium salts. For a review on suitable salts, see Handbook of Pharmaceutical Salts: Properties, Selection, and Use by Stahl and Wermuth (Wiley-VCH, 2002).

Hemisalts of acids and bases may also be formed, for example, hemisulfate and hemicalcium salts. For a review on suitable salts, see Handbook of Pharmaceutical Salts: Properties, Selection, and Use by Stahl and Wermuth (Wiley-VCH, 2002).

Pharmaceutically acceptable salts of compounds of Formula I or Formula II may be prepared by one or more of three methods:
(i) by reacting the compound of Formula I or Formula II with the desired acid or base;
(ii) by removing an acid- or base-labile protecting group from a suitable precursor of the compound of the invention or by ring-opening a suitable cyclic precursor, for example, a lactone or lactam, using the desired acid or base; or
(iii) by converting one salt of the compound of the invention to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionization in the resulting salt may vary from completely ionized to almost non-ionized.

The compounds of Formula I or Formula II, and pharmaceutically acceptable salts thereof, may exist in unsolvated and solvated forms. The term ‘solvate’ is used herein to describe a molecular complex comprising the compound of Formula I or Formula II, or a pharmaceutically acceptable salt thereof, and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together—see Chem Commun, 17, 1889-1896, by O. Almarsson and M. J. Zaworotko (2004). For a general review of multi-component complexes, see J Pharm Sci, 64 (8), 1269-1288, by Haleblian (August 1975).

Also included within the scope of the invention are active metabolites of compounds of Formula I or Formula II (including prodrugs), that is, compounds formed in vivo upon administration of the drug, often by oxidation or dealkylation. Some examples of metabolites in accordance with the invention include:
(i) where the compound of Formula I or Formula II contains a methyl group, a hydroxymethyl derivative thereof (—CH₃->—CH₂OH) and
(ii) where the compound of Formula I or Formula II contains an alkoxy group, a hydroxy derivative thereof (—OR->—OH).

The compounds of the invention may exist in a continuum of solid states ranging from fully amorphous to fully crystalline. The term ‘amorphous’ refers to a state in which the material lacks long-range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of Formula I or Formula II may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

The compounds of Formula I or Formula II may exhibit polymorphism and/or one or more kinds of isomerism (e.g., optical, geometric or tautomeric isomerism). The compounds of Formula I or Formula II may also be isotopically labelled. Such variation is implicit to the compounds of Formula I or Formula II defined as they are by reference to their structural features and therefore within the scope of the invention.

The terms “concentrated,” “evaporated,” and “concentrated in vacuo” refer to the removal of solvent at reduced pressure on a rotary evaporator with a bath temperature less than 60° C. The abbreviation “min” and “h” stand for “minutes” and “hours” respectively. The term “room temperature or ambient temperature” means a temperature between 18 to 25° C., “GCMS” refers to gas chromatography-mass spectrometry, “LCMS” refers to liquid chromatography-mass spectrometry, “UPLC” refers to ultra-performance liquid chromatography, “SFC” refers to supercritical fluid chromatography, “HPLC” refers to high-pressure liquid chromatography, “MPLC” refers to medium-pressure liquid chromatography, “TLC” refers to thin-layer chromatography, “MS” refers to mass spectrum or mass spectroscopy or mass spectrometry, “NMR” refers to nuclear magnetic resonance spectroscopy, “DCM” refers to dichloromethane, “DMSO” refers to dimethyl sulfoxide, “DME” refers to 1,2-dimethoxyethane, “EtOAc” refers to ethyl acetate, “MeOH” refers to methanol, “Ph” refers to the phenyl group, “Pr” refers to propyl, “trityl” refers to the triphenylmethyl group, “ACN” refers to acetonitrile, “DEAD” refers to diethyl azodicarboxylate, and “DIAD” refers to diisopropyl azodicarboxylate.

In general, the compounds of this invention can be made by processes which include processes analogous to those known in the chemical arts, particularly in light of the description contained herein. Certain processes for the manufacture of the compounds of this invention are provided as further features of the invention and are illustrated by the following reaction schemes. Other processes may be described in the experimental section. Specific synthetic schemes for preparation of the compounds of Formula I or Formula II are outlined below.

As used herein, the expressions “reaction-inert solvent” and “inert solvent” refer to a solvent or a mixture thereof which does not interact with starting materials, reagents, intermediates or products in a manner which adversely affects the yield of the desired product.

As an initial note, in the preparation of the Formula I or Formula II compounds it is noted that some of the preparation methods useful for the preparation of the compounds described herein may require protection of remote functionality (e.g., primary amine, secondary amine, carboxyl in Formula I or Formula II precursors). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis, John Wiley & Sons, New York, 1991.

For example, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-tert-butoxycarbonyl, benzyloxycarbonyl, and 9-fluorenylmethylenoxycarbonyl for amines and lower alkyl or benzyl esters for carboxylic acids), which are generally not chemically reactive under the reaction conditions described and can typically be removed without chemically altering other functionality in the Formula I or Formula II compound.

The compounds of Formula I or Formula II and intermediates may contain asymmetric or chiral centers, and, therefore, exist in different stereoisomeric forms. Unless specified otherwise, it is intended that all stereoisomeric forms of the compounds as well as mixtures thereof, including racemic mixtures are included herein. In addition, all geometric and positional isomers are included within the scope of the compounds. For example, if a compound incorporates a double bond or a fused ring, both the cis- and trans-forms, as well as mixtures, are embraced within the scope of the invention.

In addition, the compounds of Formula I or Formula II and intermediates embrace all atropisomers and stereoisomeric mixtures thereof, including racemic mixtures. Atropisomers include those that can be isolated as separate stereoisomers and retain their stereoisomeric purity for various lengths of time including moderate and long times. Atropisomers also include those isomers that cannot be readily separated as separate stereoisomers due to interconversion over some time period including short to moderate times.

Chiral compounds of the invention (and chiral precursors thereof) may be obtained in enantiomerically-enriched form using chromatography, typically high pressure liquid chromatography (HPLC) or supercritical fluid chromatography (SFC), on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine (DEA) or isopropylamine. Concentration of the eluent affords the enriched mixture.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a chiral HPLC column. Alternatively, the specific stereoisomers may be synthesized by using an optically active starting material, by asymmetric synthesis using optically active reagents, substrates, catalysts or solvents, or by converting one stereoisomer into the other by asymmetric transformation.

Where the compounds possess two or more stereogenic centers and the absolute or relative stereochemistry is given in the name, the designations R and S refer respectively to each stereogenic center in ascending numerical order (1, 2, 3, etc.) according to the conventional IUPAC number schemes for each molecule. Where the compounds possess one or more stereogenic centers and no stereochemistry is given in the name or structure, it is understood that the name or structure is intended to encompass all forms of the compound, including the racemic form.

The compounds of this invention may contain olefin-like double bonds. When such bonds are present, the compounds of the invention exist as cis and trans configurations and as mixtures thereof. The term “cis” refers to the orientation of two substituents with reference to each other and the plane of the ring (either both “up” or both “down”). Analogously, the term “trans” refers to the orientation of two substituents with reference to each other and the plane of the ring (the substituents being on opposite sides of the ring).

It is also possible that the intermediates and compounds of Formula I or Formula II may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the tetrazole moiety where the proton may migrate between the four ring nitrogen as follows.

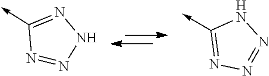

Valence tautomers include interconversions by reorganization of some of the bonding electrons.

Included within the scope of the claimed compounds present invention are all stereoisomers, geometric isomers and tautomeric forms of the compounds of Formula I or Formula II, including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Compounds of Formula I or Formula II may be prepared according to the General Schemes and Examples provided herein.

### General Schemes

In general, the compounds of this invention may be made by processes described herein and by analogous processes known to those skilled in the art. Certain processes for the manufacture of the compounds of this invention are described in the following reaction schemes. Other processes are described in the experimental section. The schemes and examples provided herein (including the corresponding description) are for illustration only. The substituent groups labelled in Schemes 1-7 are as described in this application, wherein PMB is p-methocybenzyl ether and Boc is tert-butyloxycarbonyl.

Scheme 1 refers to the preparation of compounds of Formula IA. Compounds of Formula IA can be readily prepared from intermediates IV, VI, and VIII. Intermediate IV can be prepared from an amide bond forming reaction between carboxylic acid intermediate II and amine intermediate III. Similarly, intermediates VI and VIII can be prepared from an amide bond forming reaction between intermediate II and intermediates V and VII, respectively. Amide bond forming reactions of this type can be achieved by combining a carboxylic acid (such as 1l) with an amine (such as III, V or VII) in the presence of an activating reagent (such as O-(7-azabenzotriazol-1-yl)-N,N,N,N′-tetramethyluronium hexafluorophosphate; HATU) and a base (such as N,N-diisopropylethylamine) in a suitable solvent (such as dichloromethane).

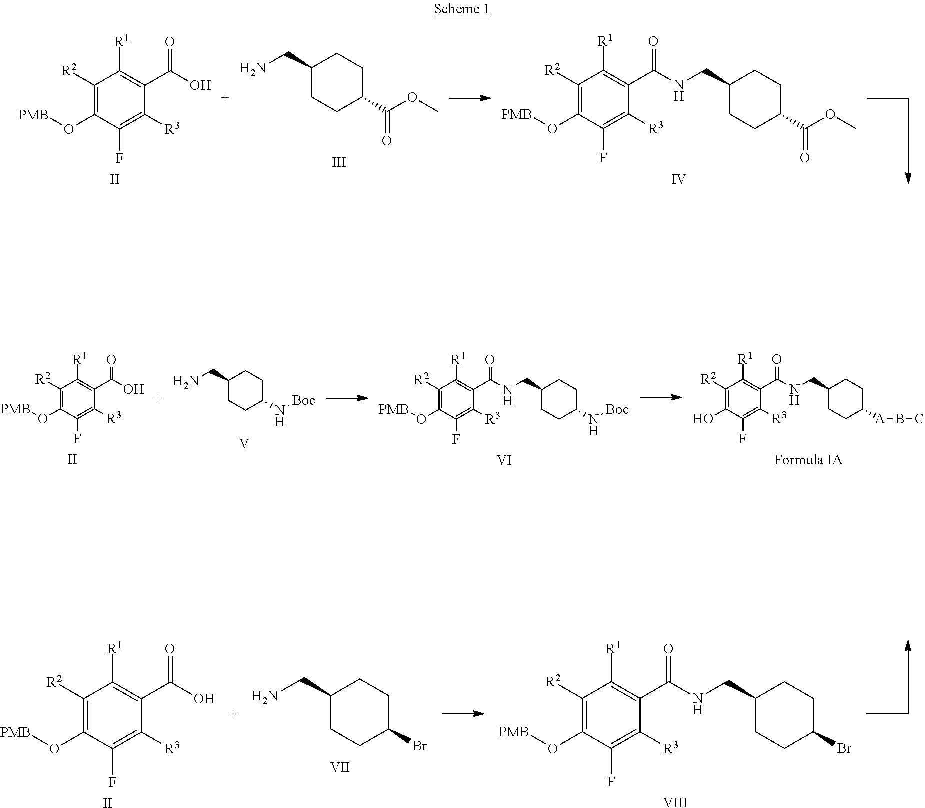

Scheme 2 refers to the preparation of compounds of Formulas IA-1, IA-2, IA-3 and IA-4 from intermediate IV. The ester in intermediate IV may be hydrolyzed to afford intermediate IX. The carboxylic acid functional group in intermediate IX can be converted to various heteroaryl rings systems by methods known to those skilled in the art. For example, intermediate IX can be reacted with aminophenols such as X under suitable conditions to afford compounds of Formula IA-1 after removal of the PMB protecting group. Alternatively, intermediate IX can be coupled with intermediates of the structure X, and the resulting compound can be further dehydrated and deprotected to afford compounds of Formula IA-2. One skilled in the art may also recognize that the carboxylic acid in intermediate IX can be converted to an alternate functional group that may have further functionality for the construction of other heteroaryl ring systems. For example, the carboxylic acid in compound IX can be converted to a bromoketone by methods known in the art to afford intermediate XII. Intermediate XII can be reacted with aminopyridines (XIII) and subsequently deprotected to prepare compounds of Formula IA-3. Alternatively, the carboxylic acid in IX can be converted to a primary amide and subsequently dehydrated to afford a nitrile-containing intermediate of structure XIV. Intermediate XIV can be reacted with hydroxylamine to afford compound XV. Compounds of structure XV can be reacted with carboxylic acids of structure XVI. The resulting compounds can be dehydrated and deprotected to form oxadiazole-containing compounds of Formula IA-4.

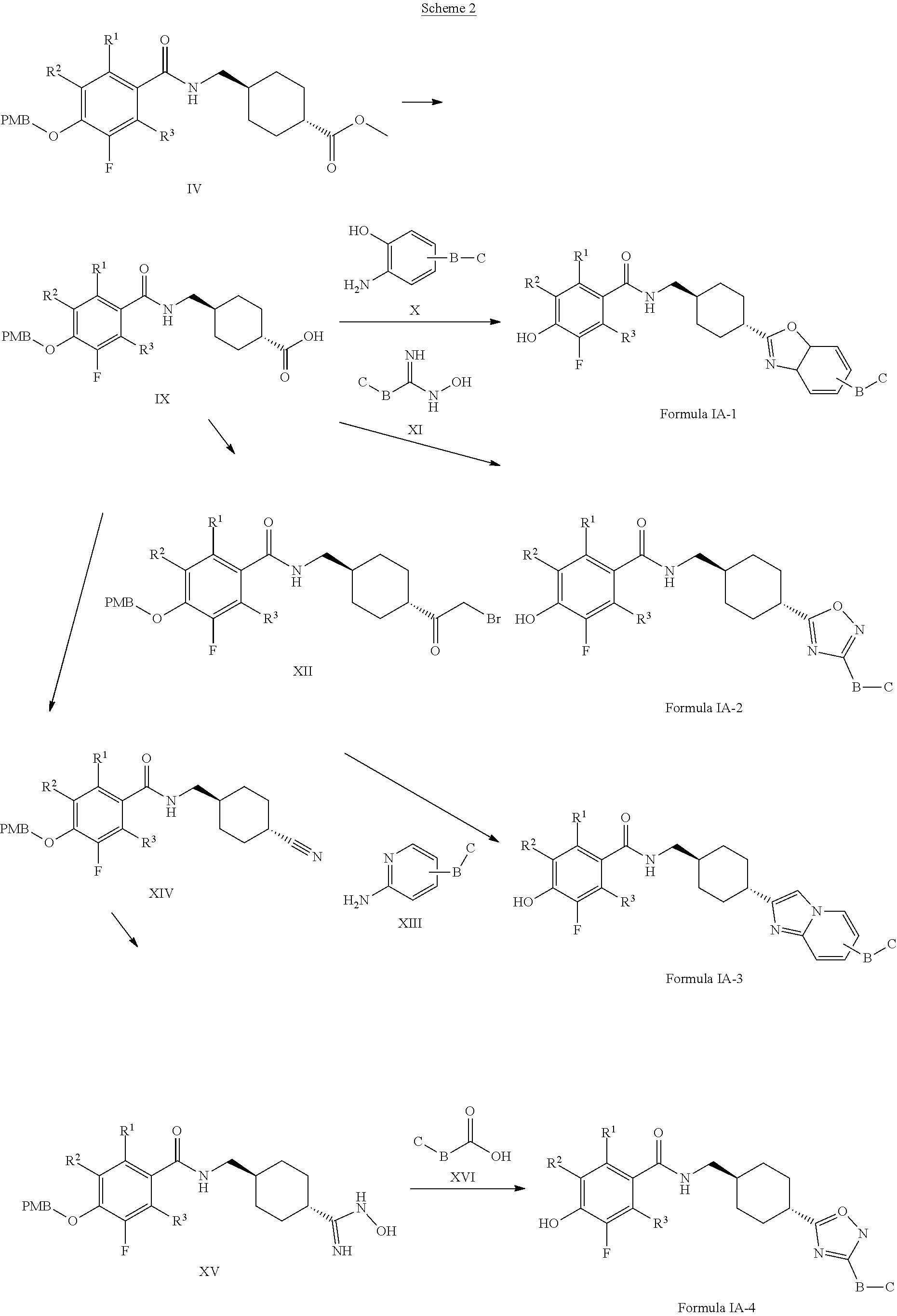

Scheme 3 refers to the preparation of compounds of Formulas IA-5 and IA-6 from intermediate VI. The Boc protecting group in intermediate VI can be selectively removed to afford intermediate XVII. Intermediate XVII can be reacted with a nitroaldehyde-containing compound (XVIII) in the presence of a trialkylphosphine to afford a compound of Formula IA-5 after removal of the PMB protecting group. Alternatively, compound XVII can be reacted with bromoester-containing compound (XIX) and subsequently deprotected to afford a compound of Formula IA-6.

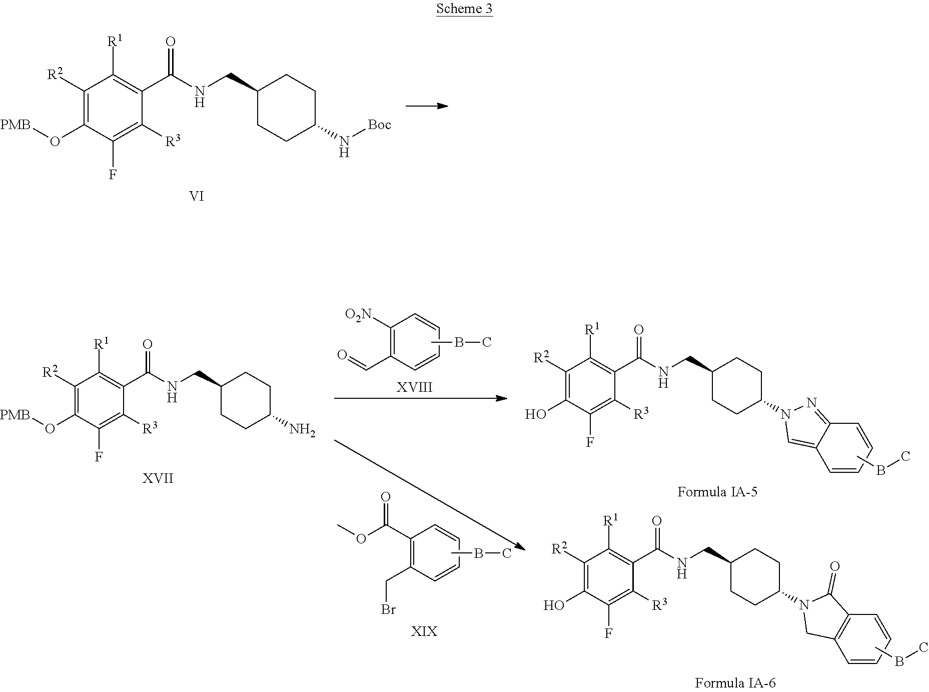

Scheme 4 refers to the preparation of compounds of Formulas IA and IA-7 from intermediate VIII. Intermediates of the structure VIII can be reacted with aryl and heteroaryl halides (XX) in the presence of suitable metal-containing catalysts and ligands to afford compounds of Formula IA after removal of the PMB protecting group. Alternatively, bromide can be displaced from intermediate VIII with sodium azide. The resulting intermediate can be reacted with an alkyne-containing compound (XXI) in the presence of a copper catalyst to afford a compound of Formula IA-7.

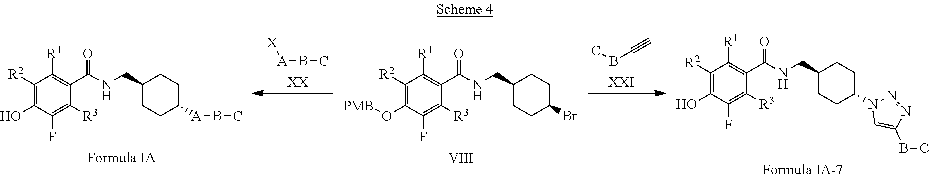

Scheme 5 refers to an alternate preparation of compounds of Formula IA-5. In some instances, compounds may be prepared by the methods described herein that contain substituents that can be utilized synthetically to prepare alternate compounds of Formula IA. For example, an intermediate of the structure XXII may be prepared by the method described for the preparation of compounds of Formula IA-5. The bromine substituent in intermediate XXII can be reacted with boronic acids (XXIII) or boronate esters (XXIII) by a Suzuki reaction to afford a compound of Formula IA-5. Additionally, compounds of structure XXII can be reacted with intermediates of structure XXIV, where B—H represents a primary or secondary amine. In this instance, XXII and XXIV can react with one another under Buchwald reaction conditions to afford another variation on compounds or Formula IA-5. Alternatively, the bromine substituent in XXII can be converted to a boronic acid (XXV; R═H) or boronate ester (XXV; R=alkyl). Compounds of the structure XXV can be reacted with aryl and heteroaryl halides of the structure XXVI to afford compounds of Formula IA-5. Additionally, compounds of structure XXV can be reacted with aromatic heterocycles bearing an N—H (XXIV′) under Cham-Lam coupling conditions to afford compounds of Formula IA-5. The example transformations provided in Scheme 5 are not intended to be comprehensive. The examples provided are just isolated examples of synthetic sequences that can be used to make modifications to the B-substituents and the C-substituents of Compounds of Formula IA. One skilled in the art will also recognize that similar transformations can be achieved with compounds containing alternate A-substituents from that depicted in Scheme 5.

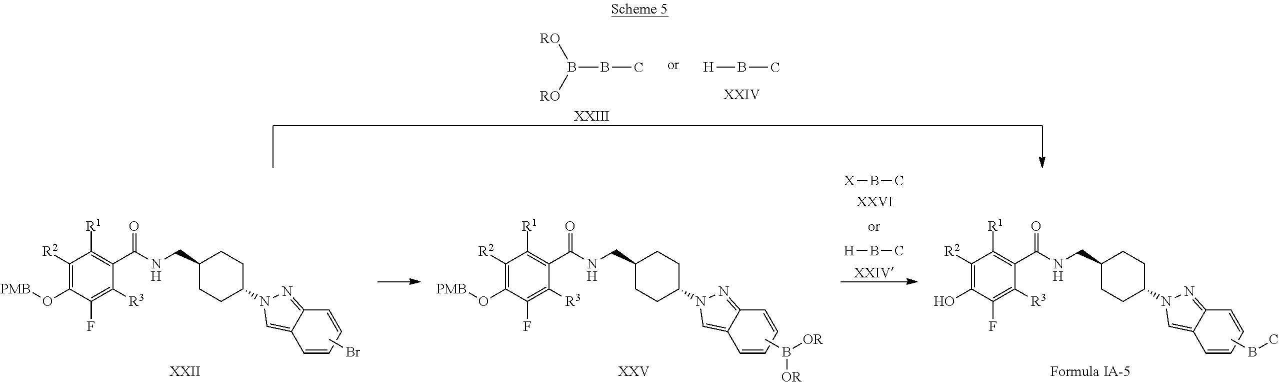

Scheme 6 refers to the preparation of compounds of Formula IB. Compounds of Formula IB can be readily prepared from intermediates XXIX and XXX. Intermediate XXIX can be prepared from an amide bond forming reaction between carboxylic acid intermediate II and amine intermediate XXVII. Similarly, intermediate XXX can be prepared from an amide bond forming reaction between intermediate II and intermediates XXVIII. Amide bond forming reactions of this type can be achieved by combining a carboxylic acid (such as II) with an amine (such as XXVII or XXVIII) in the presence of an activating reagent (such as O-(7-azabenzotriazol-1-yl)-N,N,N′N′-tetramethyluronium hexafluorophosphate; HATU) and a base (such as N,N-diisopropylethylamine) in a suitable solvent (such as dichloromethane). The preparation of compounds of Formula IB can be achieved from intermediate XXIX by methods analogous to those described for the preparation of compounds of Formula IA from intermediate IV in Scheme 2 and Scheme 5. Likewise, the preparation of compounds of Formula IB can be achieved from intermediate XXX by methods analogous to those described for the preparation of compounds of Formula IA from intermediate VI in Scheme 3 and Scheme 5.

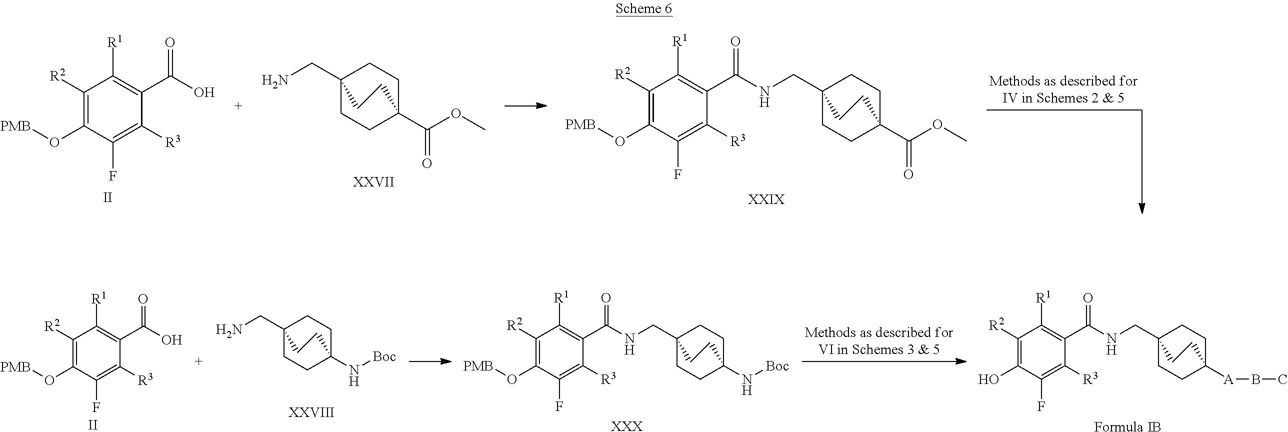

Scheme 7 refers to an alternate ordering of synthetic steps that can be utilized to prepare compounds of Formula IA or compounds of Formula IB. For example, intermediates such as XXXI, XXXII, or XXXIIII can be converted to intermediates of the structure XXXIV via methods described herein. Amine intermediates of the structure XXXIV can be reacted with a carboxylic acid of the structure II in an amide bond forming reaction. The resulting product can be deprotected to afford compounds of Formula IA. Likewise, intermediates such as XXXV and XXXVI can be converted to intermediates of the structure XXXVII. Amine intermediates of the structure XXXVII can be reacted with a carboxylic acid of the structure II and subsequently deprotected to afford compounds of Formula IB.

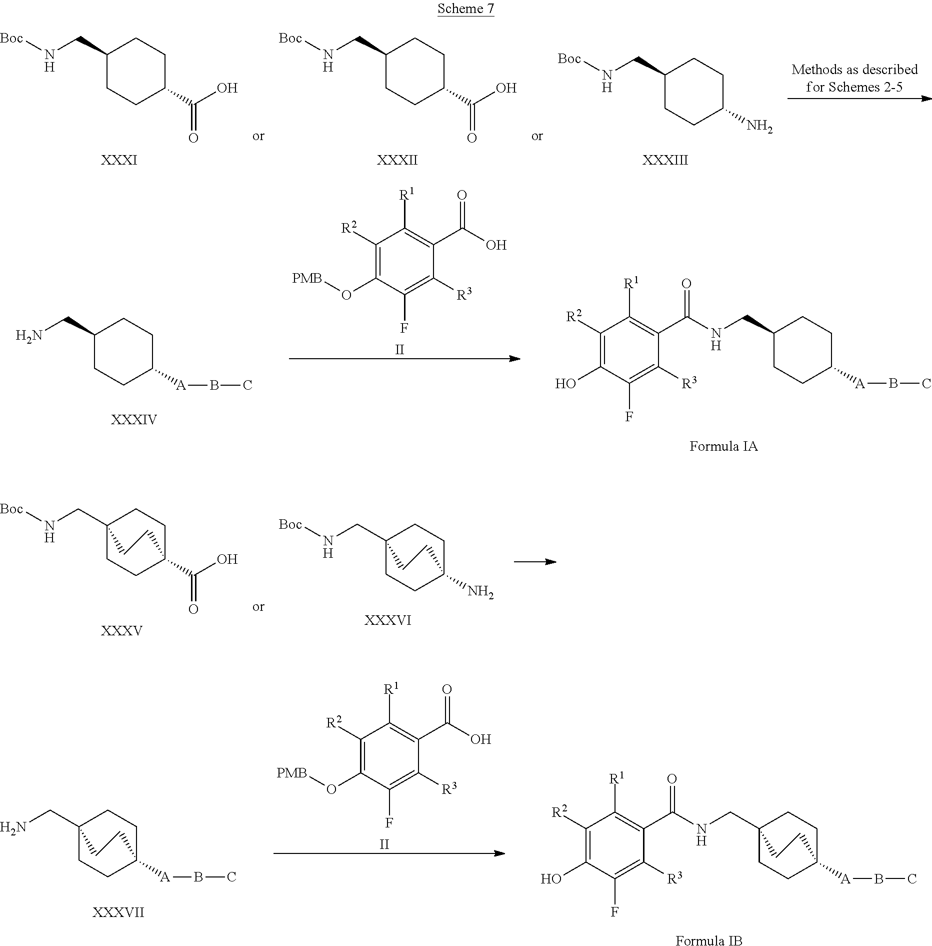

The starting materials and reagents for the above-described Formula I or Formula II compounds are also readily available or can be easily synthesized by those skilled in the art using conventional methods of organic synthesis. For example, many of the compounds used herein, are related to, or are derived from compounds in which there is a large scientific interest and commercial need, and accordingly many such compounds are commercially available or are reported in the literature or are easily prepared from other commonly available substances by methods which are reported in the literature.

This application is also directed at pharmaceutical compositions having a therapeutically effective amount of a compound of Formula I or Formula II or a pharmaceutically acceptable salt of said compound and a pharmaceutically acceptable carrier, vehicle or diluent.

In an embodiment of the invention, a method of treating fatty liver, nonalcoholic fatty liver disease, nonalcoholic steatohepatitis, nonalcoholic steatohepatitis with liver fibrosis, nonalcoholic steatohepatitis with cirrhosis or nonalcoholic steatohepatitis with cirrhosis and hepatocellular carcinoma comprising administering to a human in need of such treatment a therapeutically effective amount of the compound of Formula I or Formula II or a pharmaceutically acceptable salt of said compound.

In an embodiment of the invention, the method includes treating nonalcoholic steatohepatitis.

In an embodiment of the invention, a pharmaceutical composition comprises a therapeutically effective amount of the compound of Formula I or Formula II or a pharmaceutically acceptable salt of said compound and a pharmaceutically acceptable carrier, vehicle or diluent.

In an embodiment of the invention, a pharmaceutical combination composition comprises a therapeutically effective amount of a composition comprising: a first compound, said first compound being a compound of Formula I or Formula II or a pharmaceutically acceptable salt of said compound; a second compound, said second compound being an anti-diabetic agent; a non-alcoholic steatohepatitis treatment agent, a non-alcoholic fatty liver disease treatment agent or an anti-heart failure treatment agent; and a pharmaceutical carrier, vehicle or diluents.

In an embodiment of the invention, the non-alcoholic steatohepatitis treatment agent or non-alcoholic fatty liver disease treatment agent in the pharmaceutical combination composition is an ACC inhibitor, a KHK inhibitor, a DGAT-2 inhibitor, an FXR agonist, metformin, incretin analogues, or an incretin receptor modulator.

In an embodiment of the disclosure, the anti-diabetic agent is an SGLT-2 inhibitor, metformin, incretin analogues, an incretin receptor modulator, a DPP-4 inhibitor, or a PPAR agonist.

The compounds of this invention may also be used in conjunction with other pharmaceutical agents (e.g., antiatherosclerotic and antithrombotic agents) for the treatment of the disease/conditions described herein. This application is also directed at pharmaceutical combination compositions that include: a therapeutically effective amount of a composition having:
a first compound, said first compound being a compound of any of Formula I or Formula II or a pharmaceutically acceptable salt of said compound;
a second compound, said second compound being a treatment agent for kidney disease, an anti-diabetic agent; a non-alcoholic steatohepatitis treatment agent, a non-alcoholic fatty liver disease treatment agent or an anti-heart failure treatment agent and
a pharmaceutical carrier, vehicle or diluents.

In one embodiment, said treatment agent for kidney disease is useful for treating acute and/or chronic kidney disease.

In one embodiment, said non-alcoholic steatohepatitis treatment agent or non-alcoholic fatty liver disease treatment agent is an ACC inhibitor, a KHK inhibitor, a DGAT-2 inhibitor, an FXR agonist, a GLP-1R agonist, metformin, incretin analogues, or an incretin receptor modulator.

In another embodiment, said anti-diabetic agent is an SGLT-2 inhibitor, metformin, incretin analogues, an incretin receptor modulator, a DPP-4 inhibitor, or a PPAR agonist.

In another embodiment, said anti-diabetic agent is metformin, sitagliptin or ertuglifozin.

In another embodiment, said anti-heart failure agent is an ACE inhibitor, an angiotensin receptor blocker, an angiotensin-receptor neprilysin inhibitor, a beta adrenergic receptor blocker, a calcium channel blocker, or a vasodilator.

### Combination Agents

The compounds can be administered alone or in combination with one or more additional therapeutic agents. By “administered in combination” or “combination therapy” it is meant that a compound and one or more additional therapeutic agents are administered concurrently to the mammal being treated. When administered in combination, each component may be administered at the same time or sequentially in any order at different points in time. Thus, each component may be administered separately but sufficiently closely in time so as to provide the desired therapeutic effect. The phrases “concurrent administration,” “co-administration,” “simultaneous administration,” and “administered simultaneously” mean that the compounds are administered in combination. Thus, the methods of prevention and treatment described herein include use of combination agents.

The combination agents are administered to a mammal in a therapeutically effective amount. By “therapeutically effective amount” it is meant an amount of a compound of Formula I or Formula II that, when administered alone or in combination with an additional therapeutic agent to a mammal, is effective to treat the desired disease/condition (e.g., NASH, heart failure, kidney disease or diabetes).

Given the NASH/NAFLD activity of the compounds of this invention, they may be co-administered with other agents for the treatment of non-alcoholic steatohepatitis (NASH) and/or non-alcoholic fatty liver disease (NAFLD) and associated disease/conditions, such as Orlistat, TZDs and other insulin-sensitizing agents, FGF21 analogues, Metformin, Omega-3-acid ethyl esters (e.g., Lovaza), Fibrates, HMG-CoA reductase inhibitors (e.g., pravastatin, lovastatin, atorvastatin, simvastatin, fluvastatin, NK-104 (a.k.a. itavastatin or nisvastatin or nisbastatin) and ZD-4522 (a.k.a. rosuvastatin or atavastatin or visastatin)), Ezetimibe, proprotein convertase subtilisin kexin type-9 (PCSK9) inhibitors (e.g., evolocumab, alirocumab), Probucol, Ursodeoxycholic acid, TGR5 agonists, FXR agonists, Vitamin E, Betaine, Pentoxifylline, CB1 antagonists, Carnitine, N-acetylcysteine, Reduced glutathione, lorcaserin, the combination of naltrexone with buproprion, SGLT2 inhibitors (including dapagliflozin, canagliflozin, empagliflozin, tofogliflozin, ertugliflozin, ASP-1941, THR1474, TS-071, ISIS388626 and LX4211 as well as those in WO2010023594), Phentermine, Topiramate, GLP-1 receptor agonists, GIP receptor agonists, dual GLP-1 receptor/glucagon receptor agonists (i.e., OPK88003, MED10382, JNJ-64565111, NN9277, BI 456906), dual GLP-1 receptor/GIP receptor agonists (i.e., Tirzepatide (LY3298176), NN9423), Angiotensin-receptor blockers an acetyl-CoA carboxylase (ACC) inhibitor, a diacylglycerol O-acyltransferase 1 (DGAT-1) inhibitor, such as those described in WO09016462 or WO2010086820, AZD7687 or LCQ908, a diacylglycerol O-acyltransferase 2 (DGAT-2) inhibitor, a PNPLA3 inhibitor, an FGF21 analog, an FGF19 analog, a PPAR agonist, an FXR agonist, an AMPK activator, an SCD1 inhibitor or an MPO inhibitor.

Exemplary GLP-1 receptor agonists include liraglutide, albiglutide, exenatide, albiglutide, lixisenatide, dulaglutide, semaglutide, HM15211, LY3298176, Medi-0382, NN-9924, TTP-054, TTP-273, efpeglenatide, those described in WO2018109607, and those described in PCT/IB2019/054867 filed Jun. 11, 2019 including the following:
2-({4-[2-(4-chloro-2-fluorophenyl)-1,3-benzodioxol-4-yl]piperidin-1-yl}methyl)-1-[(2S)-oxetan-2-ylmethyl]-1H-benzimidazole-6-carboxylic acid;
2-({4-[2-(4-chloro-2-fluorophenyl)-1,3-benzodioxol-4-yl]piperidin-1-yl}methyl)-7-fluoro-1-[(2S)-oxetan-2-ylmethyl]-1H-benzimidazole-6-carboxylic acid;
2-({4-[(2S)-2-(4-chloro-2-fluorophenyl)-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(4-chloro-2-fluorophenyl)-1,3-benzodioxol-4-yl]piperidin-1-yl}methyl)-7-fluoro-1-[(2S)-oxetan-2-ylmethyl]-1H-benzimidazole-6-carboxylic acid;
2-({4-[2-(4-chloro-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(4-Cyano-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(4-Chloro-2-fluorophenyl)-2-methyl-1,3-benzodioxol-4-yl]piperidin-1-yl}methyl)-3-(1,3-oxazol-2-ylmethyl)-3H-imidazo[4,5-b]pyridine-5-carboxylic acid;
2-({4-[2-(4-chloro-2-fluorophenyl)-2-methyl-1,3-benzodioxol-4-yl]piperidin-1-yl}methyl)-1-[(1-ethyl-1H-imidazol-5-yl)methyl]-1H-benzimidazole-6-carboxylic acid;
2-({4-[2-(4-chloro-2-fluorophenyl)-2-methyl-1,3-benzodioxol-4-yl]piperidin-1-yl}methyl)-1-(1,3-oxazol-4-ylmethyl)-1H-benzimidazole-6-carboxylic acid;
2-({4-[2-(4-chloro-2-fluorophenyl)-2-methyl-1,3-benzodioxol-4-yl]piperidin-1-yl}methyl)-1-(pyridin-3-ylmethyl)-1H-benzimidazole-6-carboxylic acid;
2-({4-[2-(4-chloro-2-fluorophenyl)-2-methyl-1,3-benzodioxol-4-yl]piperidin-1-yl}methyl)-1-(1,3-oxazol-5-ylmethyl)-1H-benzimidazole-6-carboxylic acid;
2-({4-[2-(4-chloro-2-fluorophenyl)-2-methyl-1,3-benzodioxol-4-yl]piperidin-1-yl}methyl)-1-[(1-ethyl-1H-1,2,3-triazol-5-yl)methyl]-1H-benzimidazole-6-carboxylic acid;
2-({4-[2-(4-chloro-2-fluorophenyl)-2-methyl-1,3-benzodioxol-4-yl]piperidin-1-yl}methyl)-1-(1,3-oxazol-2-ylmethyl)-1H-benzimidazole-6-carboxylic acid;
2-({4-[2-(4-chloro-2-fluorophenyl)-7-fluoro-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(4-cyano-2-fluorophenyl)-2-methyl-1,3-benzodioxol-4-yl]piperidin-1-yl}methyl)-1-(1,3-oxazol-2-ylmethyl)-1H-benzimidazole-6-carboxylic acid;
2-({4-[(2S)-2-(4-chloro-2-fluorophenyl)-2-methyl-1,3-benzodioxol-4-yl]piperidin-1-yl}methyl)-7-fluoro-1-[(2S)-oxetan-2-ylmethyl]-1H-benzimidazole-6-carboxylic acid;
2-({4-[(2S)-2-(4-chloro-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(4-chloro-2-fluorophenyl)-2-methyl-1,3-benzodioxol-4-yl]piperidin-1-yl}methyl)-7-fluoro-1-[(2S)-oxetan-2-ylmethyl]-1H-benzimidazole-6-carboxylic acid;
2-({4-[(2S)-2-(4-Cyano-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(4-chloro-2-fluorophenyl)-2-methyl-1,3-benzodioxol-4-yl]piperidin-1-yl}methyl)-1-[(1-ethyl-1H-imidazol-5-yl)methyl]-1H-benzimidazole-6-carboxylic acid;
2-({4-[(2R)-2-(4-Cyano-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2R)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2R)-2-(4-chloro-2-fluorophenyl)-2-methyl-1,3-benzodioxol-4-yl]piperidin-1-yl}methyl)-1-[(1-ethyl-1H-imidazol-5-yl)methyl]-1H-benzimidazole-6-carboxylic acid;
2-({4-[2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2R)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid, DIAST-X2; and
2-[(4-{6-[(4-Cyano-2-fluorobenzyl)oxy]pyridin-2-yl}piperidin-1-yl)methyl]-1-[(2S)-oxetan-2-ylmethyl]-1H-benzimidazole-6-carboxylic acid, or pharmaceutically acceptable salts thereof.

Exemplary ACC inhibitors include 4-(4-[(1-isopropyl-7-oxo-1,4,6,7-tetrahydro-1′H-spiro[indazole-5,4′-piperidin]-1′-yl)carbonyl]-6-methoxypyridin-2-yl)benzoic acid; and firsocostat (GS-0976) and pharmaceutically acceptable salts thereof.

Exemplary FXR Agonists include tropifexor (2-[(1R,3R,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid); cilofexor (GS-9674); obeticholic acid; LY2562175; Met409; TERN-101; and EDP-305 and pharmaceutically acceptable salts thereof.

Exemplary DGAT2 inhibitors include (S)-2-(5-((3-ethoxypyridin-2-yl)oxy)pyridin-3-yl)-N-(tetrahydrofuran-3-yl)pyrimidine-5-carboxamide;
2-(5-((3-ethoxy-5-fluoropyridin-2-yl)oxy)pyridin-3-yl)-N-((3R,4S)-4-fluoropiperidin-3-yl)pyrimidine-5-carboxamide;
2-(5-((3-ethoxy-5-fluoropyridin-2-yl)oxy)pyridin-3-yl)-N-((3S,5S)-5-fluoropiperidin-3-yl)pyrimidine-5-carboxamide;
2-(5-((3-ethoxypyridin-2-yl)oxy)pyridin-3-yl)-N-((3R,4S)-4-fluoropiperidin-3-yl)pyrimidine-5-carboxamide;
2-(5-((3-ethoxypyridin-2-yl)oxy)pyridin-3-yl)-N-((3R,4R)-4-fluoropiperidin-3-yl)pyrimidine-5-carboxamide;
2-(5-((3-ethoxy-5-fluoropyridin-2-yl)oxy)pyridin-3-yl)-N-((3R,4R)-4-fluoropiperidin-3-yl)pyrimidine-5-carboxamide; and
2-(5-((3-ethoxypyridin-2-yl)oxy)pyridin-3-yl)-N-((3S,5S)-5-fluoropiperidin-3-yl)pyrimidine-5-carboxamide, or a pharmaceutically acceptable salt thereof.

Exemplary KHK inhibitors include [(1R,5S,6R)-3-{2-[(2S)-2-methylazetidin-1-yl]-6-(trifluoromethyl)pyrimidin-4-yl}-3-azabicyclo[3.1.0]hex-6-yl]acetic acid and pharmaceutically acceptable salts thereof.

Given the anti-diabetic activity of the compounds of this invention they may be co-administered with other anti-diabetic agents. Suitable anti-diabetic agents include insulin, metformin, GLP-1 receptor agonists (described herein above), an acetyl-CoA carboxylase (ACC) inhibitor (described herein above), SGLT2 inhibitors (described herein above), monoacylglycerol O-acyltransferase inhibitors, phosphodiesterase (PDE)-10 inhibitors, AMPK activators, sulfonylureas (e.g., acetohexamide, chlorpropamide, diabinese, glibenclamide, glipizide, glyburide, glimepiride, gliclazide, glipentide, gliquidone, glisolamide, tolazamide, and tolbutamide), meglitinides, α-amylase inhibitors (e.g., tendamistat, trestatin and AL-3688), an α-glucoside hydrolase inhibitor (e.g., acarbose), α-glucosidase inhibitors (e.g., adiposine, camiglibose, emiglitate, miglitol, voglibose, pradimicin-Q, and salbostatin), PPARγ agonists (e.g., balaglitazone, ciglitazone, darglitazone, englitazone, isaglitazone, pioglitazone and rosiglitazone), PPAR α/γ agonists (e.g., CLX-0940, GW-1536, GW-1929, GW-2433, KRP-297, L-796449, LR-90, MK-0767 and SB-219994), protein tyrosine phosphatase-1B (PTP-1B) inhibitors (e.g., trodusquemine, hyrtiosal extract, and compounds disclosed by Zhang, S., et al., Drug Discovery Today, 12(9/10), 373-381 (2007)), SIRT-1 activators (e.g., resveratrol, GSK2245840 or GSK184072), dipeptidyl peptidease IV (DPP-IV) inhibitors (e.g., those in WO2005116014, sitagliptin, vildagliptin, alogliptin, dutogliptin, linagliptin and saxagliptin), insulin secreatagogues, fatty acid oxidation inhibitors, A2 antagonists, c-jun amino-terminal kinase (JNK) inhibitors, glucokinase activators (GKa) such as those described in WO2010103437, WO201010343f8, WO2010013161, WO2007122482, TTP-399, TTP-355, TTP-547, AZD1656, ARRY403, MK-0599, TAK-329, AZD5658 or GKM-001, insulin, insulin mimetics, glycogen phosphorylase inhibitors (e.g., GSK1362885), VPAC2 receptor agonists, glucagon receptor modulators such as those described in Demong, D. E. et al. Annual Reports in Medicinal Chemistry 2008, 43, 119-137, GPR119 modulators, particularly agonists, such as those described in WO2010140092, WO2010128425, WO2010128414, WO2010106457, Jones, R. M. et al. in Medicinal Chemistry 2009, 44, 149-170 (e.g., MBX-2982, GSK1292263, APD597 and PSN821), FGF21 derivatives or analogues such as those described in Kharitonenkov, A. et al. et al., Current Opinion in Investigational Drugs 2009, 10(4)359-364, TGR5 (also termed GPBAR1) receptor modulators, particularly agonists, such as those described in Zhong, M., Current Topics in Medicinal Chemistry, 2010, 10(4), 386-396 and INT777, GPR40 agonists, such as those described in Medina, J. C., Annual Reports in Medicinal Chemistry, 2008, 43, 75-85, including but not limited to TAK-875, GPR120 modulators, particularly agonists, high affinity nicotinic acid receptor (HM74A) activators, and SGLT1 inhibitors, such as GSK1614235. A further representative listing of anti-diabetic agents that can be combined with the compounds of this application can be found, for example, at page 28, line 35 through page 30, line 19 of WO2011005611.

Other anti-diabetic agents could include inhibitors or modulators of carnitine palmitoyl transferase enzymes, inhibitors of fructose 1,6-diphosphatase, inhibitors of aldose reductase, mineralocorticoid receptor inhibitors, inhibitors of TORC2, inhibitors of CCR2 and/or CCR5, inhibitors of PKC isoforms (e.g., PKCα, PKCβ, PKCγ), inhibitors of fatty acid synthetase, inhibitors of serine palmitoyl transferase, modulators of GPR81, GPR39, GPR43, GPR41, GPR105, Kv1.3, retinol binding protein 4, glucocorticoid receptor, somatostatin receptors (e.g., SSTR1, SSTR2, SSTR3 and SSTR5), inhibitors or modulators of PDHK2 or PDHK4, inhibitors of MAP4K4, modulators of IL1 family including IL1beta, modulators of RXRalpha. In addition suitable anti-diabetic agents include mechanisms listed by Carpino, P. A., Goodwin, B. Expert Opin. Ther. Pat, 2010, 20(12), 1627-51.

Given the anti-heart failure activity of the compounds of this application, they may be co-administered with other anti-heart failure agents such as ACE inhibitors (e.g., captopril, enalapril, fosinopril, Lisinopril, perindopril, quinapril, Ramipril, trandolapril), Angiotensin II receptor blockers (e.g., Candesartan, Losartan, Valsartan), Angiotensin-receptor neprilysin inhibitors (sacubitril/valsartan), I_{f} channel blocker Ivabradine, Beta-Adrenergic blocking agents (e.g., bisoprolol, metoprolol succinate, carvedilol), SGLT2 inhibitors, Aldosterone antagonists (e.g., spironolactone, eplerenone), cardiac myosin activator (e.g., omecamtiv mecarbil), guanylate cyclase stimulator (e.g., vericiguat), cardiac myosin inhibitor (e.g., mavacamten), SERCA2a activator (e.g., istaroxime), hydralazine and isosorbide dinitrate, diuretics (e.g., furosemide, bumetanide, torsemide, chlorothiazide, amiloride, hydrochlorothiazide, Indapamide, Metolazone, Triamterene), or digoxin.

The compounds of Formula I or Formula II may also be used in combination with antihypertensive agents and such antihypertensive activity is readily determined by those skilled in the art according to standard assays (e.g., blood pressure measurements). Examples of suitable anti-hypertensive agents include: alpha adrenergic blockers; beta adrenergic blockers; calcium channel blockers (e.g., diltiazem, verapamil, nifedipine and amlodipine); vasodilators (e.g., hydralazine), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, torsemide, furosemide, musolimine, bumetanide, triamtrenene, amiloride, spironolactone); renin inhibitors; ACE inhibitors (e.g., captopril, zofenopril, fosinopril, enalapril, ceranopril, cilazopril, delapril, pentopril, quinapril, ramipril, lisinopril); AT-1 receptor antagonists (e.g., losartan, irbesartan, valsartan); ET receptor antagonists (e.g., sitaxsentan, atrsentan and compounds disclosed in U.S. Pat. Nos. 5,612,359 and 6,043,265); Dual ET/AII antagonist (e.g., compounds disclosed in WO 00/01389); neutral endopeptidase (NEP) inhibitors; vasopepsidase inhibitors (dual NEP-ACE inhibitors) (e.g., gemopatrilat and nitrates). An exemplary antianginal agent is ivabradine.

Examples of suitable calcium channel blockers (L-type or T-type) include diltiazem, verapamil, nifedipine and amlodipine and mybefradil.

Examples of suitable cardiac glycosides include digitalis and ouabain.

In one embodiment, a Formula I or Formula II compound may be co-administered with one or more diuretics. Examples of suitable diuretics include (a) loop diuretics such as furosemide (such as LASIX™), torsemide (such as DEMADEX™), bemetanide (such as BUMEX™), and ethacrynic acid (such as EDECRIN™); (b) thiazide-type diuretics such as chlorothiazide (such as DIURIL™, ESIDRIX™ or HYDRODIURIL™), hydrochlorothiazide (such as MICROZIDE™ or ORETIC™), benzthiazide, hydroflumethiazide (such as SALURON™), bendroflumethiazide, methychlorthiazide, polythiazide, trichlormethiazide, and indapamide (such as LOZOL™); (c) phthalimidine-type diuretics such as chlorthalidone (such as HYGROTON™), and metolazone (such as ZAROXOLYN™); (d) quinazoline-type diuretics such as quinethazone; and (e) potassium-sparing diuretics such as triamterene (such as DYRENIUM™), and amiloride (such as MIDAMOR™ or MODURETIC™).

In another embodiment, a compound of Formula I or Formula II may be co-administered with a loop diuretic. In still another embodiment, the loop diuretic is selected from furosemide and torsemide. In still another embodiment, one or more compounds of Formula I or Formula II may be co-administered with furosemide. In still another embodiment, one or more compounds of Formula I or Formula II may be co-administered with torsemide which may optionally be a controlled or modified release form of torsemide.

In another embodiment, a compound of Formula I or Formula II may be co-administered with a thiazide-type diuretic. In still another embodiment, the thiazide-type diuretic is selected from the group consisting of chlorothiazide and hydrochlorothiazide. In still another embodiment, one or more compounds of Formula I or Formula II may be co-administered with chlorothiazide. In still another embodiment, one or more compounds of Formula I or Formula II may be co-administered with hydrochlorothiazide.

In another embodiment, one or more compounds of Formula I or Formula II may be co-administered with a phthalimidine-type diuretic. In still another embodiment, the phthalimidine-type diuretic is chlorthalidone.

Examples of suitable mineralocorticoid receptor antagonists include spironolactone and eplerenone.

Examples of suitable phosphodiesterase inhibitors include: PDE Ill inhibitors (such as cilostazol); and PDE V inhibitors (such as sildenafil).

Those skilled in the art will recognize that the compounds of this invention may also be used in conjunction with other cardiovascular or cerebrovascular treatments including PCI, stenting, drug-eluting stents, stem cell therapy and medical devices such as implanted pacemakers, defibrillators, or cardiac resynchronization therapy.

The compounds of Formula I or Formula II may also be used in combination with drugs used in the management of chronic kidney disease including phosphate binders (e.g., sucroferric oxyhydroxide, sevelamer, calcium acetate), sodium bicarbonate, erythropoietin-stimulating agents, oral or intravenous iron agents (e.g., iron sucrose, ferric carboxymaltose, ferumoxytol), potassium binders, calcitriol, or SGLT2 inhibitors (e.g., dapagliflozin, empagliflozin, or other SGLT2 inhibitors recited herein).

Particularly when provided as a single dosage unit, the potential exists for a chemical interaction between the combined active ingredients. For this reason, when a Formula I or Formula II compound and a second therapeutic agent are combined in a single dosage unit they may be formulated such that although the active ingredients are combined in a single dosage unit, the physical contact between the active ingredients is minimized (that is, reduced). For example, one active ingredient may be enteric coated. By enteric coating one of the active ingredients, it is possible not only to minimize the contact between the combined active ingredients, but also, it is possible to control the release of one of these components in the gastrointestinal tract such that one of these components is not released in the stomach but rather is released in the intestines. One of the active ingredients may also be coated with a material that effects a sustained release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low viscosity grade of hydroxypropyl methylcellulose (HPMC) or other appropriate materials as known in the art, in order to further separate the active components. The polymer coating serves to form an additional barrier to interaction with the other component.

Sustained-release preparations or formulations may be used. Suitable examples of sustained-release preparations or formulations include semi-permeable matrices of solid hydrophobic polymers containing the compound of the invention, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as those used in LUPRON DEPOT™ (injectable microspheres composed of lactic acid-glycolic acid copolymer and leuprolide acetate), sucrose acetate isobutyrate, and poly-D-(−)-3-hydroxybutyric acid.

These as well as other ways of minimizing contact between the components of combination products, whether administered in a single dosage form or administered in separate forms but at the same time by the same manner, will be readily apparent to those skilled in the art, once armed with the present disclosure.

In combination therapy treatment, both the compounds of this invention and the other drug therapies are administered to mammals (e.g., humans, male or female) by conventional methods.

The Formula I or Formula II compound of this invention, their prodrugs and the salts of such compounds and prodrugs are all adapted to therapeutic use as agents that inhibit and/or degrade HSD17B13 in mammals, particularly humans and thus are useful for the treatment of the various conditions (e.g., those described herein) in which such action is implicated.

The disease/conditions that can be treated with compounds of Formula I or Formula II, include, but are not limited to NASH/NAFLD, diabetes, kidney disease, and heart failure and associated disease/conditions.

Accordingly, given the positive correlation between activation of HSD17B13 with the development of NASH/NAFLD and associated disease/conditions, Formula I or Formula II compounds of this invention, their prodrugs and the salts of such compounds and prodrugs, by virtue of their pharmacologic action, are useful for the prevention, arrestment and/or regression of fatty liver, nonalcoholic fatty liver disease, nonalcoholic steatohepatitis, nonalcoholic steatohepatitis with liver fibrosis, nonalcoholic steatohepatitis with cirrhosis, or nonalcoholic steatohepatitis with cirrhosis and hepatocellular carcinoma.

Administration of the compounds of this invention can be via any method that delivers a compound of this invention systemically and/or locally. These methods include oral routes, parenteral, intraduodenal routes, buccal, intranasal etc. Generally, the compounds of this invention are administered orally, but parenteral administration (e.g., intravenous, intramuscular, subcutaneous or intramedullary) may be utilized, for example, where oral administration is inappropriate for the target or where the patient is unable to ingest the drug.

For administration to human patients, an oral daily dose of the compounds herein may be in the range 1 mg to 5000 mg depending, of course, on the mode of and frequency of administration, the disease state, and the age and condition of the patient, etc. An oral daily dose is in the range of 3 mg to 3000 mg may be used. A further oral daily dose is in the range of 5 mg to 1000 mg. For convenience, the compounds of Formula I or Formula II can be administered in a unit dosage form. If desired, multiple doses per day of the unit dosage form can be used to increase the total daily dose. The unit dosage form, for example, may be a tablet or capsule containing about 0.1, 0.5, 1, 5, 10, 15, 20, 25, 30, 35, 40, 45, 50, 55, 60, 65, 70, 75, 80, 85, 90, 95, 100, 125, 150, 175, 200, 250, 500, or 1000 mg of the compound. The total daily dose may be administered in single or divided doses and may, at the physician's discretion, fall outside of the typical ranges given herein.

For administration to human patients, an infusion daily dose of the compounds herein may be in the range 1 mg to 2000 mg depending, of course, on the mode of and frequency of administration, the disease state, and the age and condition of the patient, etc. A further infusion daily dose is in the range of 5 mg to 1000 mg. The total daily dose may be administered in single or divided doses and may, at the physician's discretion, fall outside of the typical ranges given herein.

These compounds may also be administered to animals other than humans, for example, for the indications detailed above. The precise dosage administered of each active ingredient will vary depending upon any number of factors, including but not limited to, the type of animal and type of disease state being treated, the age of the animal, and the route(s) of administration.

A dosage of the combination pharmaceutical agents to be used in conjunction with the Formula I or Formula II compound is used that is effective for the indication being treated. Such dosages can be determined by standard assays such as those referenced above and provided herein. The combination agents may be administered simultaneously or sequentially in any order.

These dosages are based on an average human subject having a weight of about 60 kg to 70 kg. The physician will readily be able to determine doses for subjects whose weight falls outside this range, such as infants and the elderly.

Dosage regimens may be adjusted to provide the optimum desired response. For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention is dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, intra-patient dose-escalation may be used as determined by the skilled artisan. Determining appropriate dosages and regiments for administration of the chemotherapeutic agent are well-known in the relevant art and would be understood to be encompassed by the skilled artisan once provided the teachings disclosed herein.

This application further comprises use of a compound of Formula I or Formula II for use as a medicament (such as a unit dosage tablet or unit dosage capsule). In another embodiment, this application comprises the use of a compound of Formula I or Formula II for the manufacture of a medicament (such as a unit dosage tablet or unit dosage capsule) to treat one or more of the conditions previously identified in the above sections discussing methods of treatment.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The compounds of the invention or combinations can be administered alone but will generally be administered in an admixture with one or more suitable pharmaceutical excipients, adjuvants, diluents or carriers known in the art and selected with regard to the intended route of administration and standard pharmaceutical practice. The compound of the invention or combination may be formulated to provide immediate-, delayed-, modified-, sustained-, pulsed- or controlled-release dosage forms depending on the desired route of administration and the specificity of release profile, commensurate with therapeutic needs.

The pharmaceutical composition comprises a compound of the invention or a combination in an amount generally in the range of from about 1% to about 75%, 80%, 85%, 90% or even 95% (by weight) of the composition, usually in the range of about 1%, 2% or 3% to about 50%, 60% or 70%, more frequently in the range of about 1%, 2% or 3% to less than 50% such as about 25%, 30% or 35%.

Methods of preparing various pharmaceutical compositions with a specific amount of active compound are known to those skilled in this art. For examples, see Remington: The Practice of Pharmacy, Lippincott Williams and Wilkins, Baltimore Md. 20.sup.th ed. 2000.

Compositions suitable for parenteral injection generally include pharmaceutically acceptable sterile aqueous or nonaqueous solutions, dispersions, suspensions, or emulsions, and sterile powders for reconstitution into sterile injectable solutions or dispersions. Examples of suitable aqueous and nonaqueous carriers or diluents (including solvents and vehicles) include water, ethanol, polyols (propylene glycol, polyethylene glycol, glycerol, and the like), suitable mixtures thereof, triglycerides including vegetable oils such as olive oil, and injectable organic esters such as ethyl oleate. A preferred carrier is Miglyol® brand caprylic/capric acid ester with glycerin or propylene glycol (e.g., Miglyol® 812, Miglyol® 829, Miglyol® 840) available from Condea Vista Co., Cranford, N.J. Proper fluidity can be maintained, for example, by the use of a coating such as lecithin, by the maintenance of the required particle size in the case of dispersions, and by the use of surfactants.

These compositions for parenteral injection may also contain excipients such as preserving, wetting, emulsifying, and dispersing agents. Prevention of microorganism contamination of the compositions can be accomplished with various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents capable of delaying absorption, for example, aluminum monostearate and gelatin.

Solid dosage forms for oral administration include capsules, tablets, chews, lozenges, pills, powders, and multi-particulate preparations or formulations (granules). In such solid dosage forms, a compound of Formula I or Formula II or a combination is admixed with at least one inert excipient, diluent or carrier. Suitable excipients, diluents or carriers include materials such as sodium citrate or dicalcium phosphate and/or (a) one or more fillers or extenders (e.g., microcrystalline cellulose (available as Avicel® from FMC Corp.) starches, lactose, sucrose, mannitol, silicic acid, xylitol, sorbitol, dextrose, calcium hydrogen phosphate, dextrin, alpha-cyclodextrin, beta-cyclodextrin, polyethylene glycol, medium chain fatty acids, titanium oxide, magnesium oxide, aluminum oxide and the like); (b) one or more binders (e.g., carboxymethylcellulose, methylcellulose, hydroxypropylcellulose, hydroxypropylmethylcellulose, gelatin, gum arabic, ethyl cellulose, polyvinyl alcohol, pullulan, pregelatinized starch, agar, tragacanth, alginates, gelatin, polyvinylpyrrolidone, sucrose, acacia and the like); (c) one or more humectants (e.g., glycerol and the like); (d) one or more disintegrating agents (e.g., agar-agar, calcium carbonate, potato or tapioca starch, alginic acid, certain complex silicates, sodium carbonate, sodium lauryl sulphate, sodium starch glycolate (available as Explotab® from Edward Mendell Co.), cross-linked polyvinyl pyrrolidone, croscarmellose sodium A-type (available as Ac-di-sol®), polyacrilin potassium (an ion exchange resin) and the like); (e) one or more solution retarders (e.g., paraffin and the like); (f) one or more absorption accelerators (e.g., quaternary ammonium compounds and the like); (g) one or more wetting agents (e.g., cetyl alcohol, glycerol monostearate and the like); (h) one or more adsorbents (e.g., kaolin, bentonite and the like); and/or (i) one or more lubricants (e.g., talc, calcium stearate, magnesium stearate, stearic acid, polyoxyl stearate, cetanol, talc, hydrogenated castor oil, sucrose esters of fatty acid, dimethylpolysiloxane, microcrystalline wax, yellow beeswax, white beeswax, solid polyethylene glycols, sodium lauryl sulfate and the like). In the case of capsules and tablets, the dosage forms may also comprise buffering agents.

Solid compositions of a similar type may also be used as fillers in soft or hard filled gelatin capsules using such excipients as lactose or milk sugar, as well as high molecular weight polyethylene glycols, and the like.

Solid dosage forms such as tablets, dragees, capsules, and granules may be prepared with coatings and shells, such as enteric coatings and others well known in the art. They may also contain opacifying agents, and can also be of such composition that they release the compound of Formula I or Formula II and/or the additional pharmaceutical agent in a delayed manner. Examples of embedding compositions that can be used are polymeric substances and waxes. The drug may also be in micro-encapsulated form, if appropriate, with one or more of the above-mentioned excipients.

For tablets, the active agent will typically comprise less than 50% (by weight) of the formulation, for example less than about 10% such as 5% or 2.5% by weight. The predominant portion of the formulation comprises fillers, diluents, disintegrants, lubricants and optionally, flavors. The composition of these excipients is well known in the art. Frequently, the fillers/diluents will comprise mixtures of two or more of the following components: microcrystalline cellulose, mannitol, lactose (all types), starch, and di-calcium phosphate. The filler/diluent mixtures typically comprise less than 98% of the formulation and preferably less than 95%, for example 93.5%.

Preferred disintegrants include Ac-di-sol®, Explotab®, starch and sodium lauryl sulphate. When present, a disintegrant will usually comprise less than 10% by weight of the formulation or less than 5%, for example about 3%. A preferred lubricant is magnesium stearate. When present a lubricant will usually comprise less than 5% by weight of the formulation or less than 3%, for example about 1%.

Tablets may be manufactured by standard tableting processes, for example, direct compression or a wet, dry or melt granulation, melt congealing process and extrusion. The tablet cores may be mono or multi-layer(s) and can be coated with appropriate overcoats known in the art.

Liquid dosage forms for oral administration include pharmaceutically acceptable emulsions, solutions, suspensions, syrups, and elixirs. In addition to the compound of Formula I or Formula II or the combination, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed oil, groundnut oil, corn germ oil, olive oil, castor oil, sesame seed oil and the like), Miglyol® (available from CONDEA Vista Co., Cranford, N.J.), glycerol, tetrahydrofurfuryl alcohol, polyethylene glycols and fatty acid esters of sorbitan, or mixtures of these substances, and the like.

Besides such inert diluents, the composition may also include excipients, such as wetting agents, emulsifying and suspending agents, sweetening, flavoring, and perfuming agents.

Oral liquid forms of the compounds of the invention or combinations include solutions, wherein the active compound is fully dissolved. Examples of solvents include all pharmaceutically precedented solvents suitable for oral administration, particularly those in which the compounds of the invention show good solubility, e.g., polyethylene glycol, polypropylene glycol, edible oils and glyceryl- and glyceride-based systems. Glyceryl- and glyceride-based systems may include, for example, the following branded products (and corresponding generic products): Captex® 355 EP (glyceryl tricaprylate/caprate, from Abitec, Columbus Ohio), Crodamol™ GTC/C (medium chain triglyceride, from Croda, Cowick Hall, UK) or Labrafac™ CC (medium chain triglycerides, from Gattefosse), Captex® 500P (glyceryl triacetate i.e., triacetin, from Abitec), Capmul® MCM (medium chain mono- and diglycerides, from Abitec), Miglyol® 812 (caprylic/capric triglyceride, from Condea, Cranford N.J.), Migyol® 829 (caprylic/capric/succinic triglyceride, from Condea), Migyol® 840 (propylene glycol dicaprylate/dicaprate, from Condea), Labrafil® M1944CS (oleoyl macrogol-6 glycerides, from Gattefosse), Peceol™ (glyceryl monooleate, from Gattefosse) and Maisine® 35-1 (glyceryl monooleate, from Gattefosse). Of particular interest are the medium chain (about C₈ to C₁₀) triglyceride oils. These solvents frequently make up the predominant portion of the composition, i.e., greater than about 50% by weight, usually greater than about 80%, for example about 95% or 99%. Adjuvants and additives may also be included with the solvents principally as taste-mask agents, palatability and flavoring agents, antioxidants, stabilizers, texture and viscosity modifiers and solubilizers.

Suspensions, in addition to the compound of Formula I or Formula II or the combination, may further comprise carriers such as suspending agents, e.g., ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal or vaginal administration preferably comprise suppositories, which can be prepared by mixing a compound of Formula I or Formula II or a combination with suitable non-irritating excipients or carriers, such as cocoa butter, polyethylene glycol or a suppository wax which are solid at ordinary room temperature, but liquid at body temperature, and therefore, melt in the rectum or vaginal cavity thereby releasing the active component(s).

Dosage forms for topical administration of the compounds of Formula I or Formula II or combinations include ointments, creams, lotions, powders and sprays. The drugs are admixed with a pharmaceutically acceptable excipient, diluent or carrier, and any preservatives, buffers, or propellants that may be required.

Many of the present compounds are poorly soluble in water, e.g., less than about 1 μg/mL. Therefore, liquid compositions in solubilizing, non-aqueous solvents such as the medium chain triglyceride oils discussed above are a preferred dosage form for these compounds.

Solid amorphous dispersions, including dispersions formed by a spray-drying process, are also a preferred dosage form for the poorly soluble compounds of the invention. By “solid amorphous dispersion” is meant a solid material in which at least a portion of the poorly soluble compound is in the amorphous form and dispersed in a water-soluble polymer. By “amorphous” is meant that the poorly soluble compound is not crystalline. By “crystalline” is meant that the compound exhibits long-range order in three dimensions of at least 100 repeat units in each dimension. Thus, the term amorphous is intended to include not only material which has essentially no order, but also material which may have some small degree of order, but the order is in less than three dimensions and/or is only over short distances. Amorphous material may be characterized by techniques known in the art such as powder x-ray diffraction (PXRD) crystallography, solid state NMR, or thermal techniques such as differential scanning calorimetry (DSC).

Preferably, at least a major portion (i.e., at least about 60 wt. %) of the poorly soluble compound in the solid amorphous dispersion is amorphous. The compound can exist within the solid amorphous dispersion in relatively pure amorphous domains or regions, as a solid solution of the compound homogeneously distributed throughout the polymer or any combination of these states or those states that lie intermediate between them. Preferably, the solid amorphous dispersion is substantially homogeneous so that the amorphous compound is dispersed as homogeneously as possible throughout the polymer. As used herein, “substantially homogeneous” means that the fraction of the compound that is present in relatively pure amorphous domains or regions within the solid amorphous dispersion is relatively small, on the order of less than 20 wt. %, and preferably less than 10 wt. % of the total amount of drug.

Water-soluble polymers suitable for use in the solid amorphous dispersions should be inert, in the sense that they do not chemically react with the poorly soluble compound in an adverse manner, are pharmaceutically acceptable, and have at least some solubility in aqueous solution at physiologically relevant pHs (e.g., 1-8). The polymer can be neutral or ionizable, and should have an aqueous-solubility of at least 0.1 mg/mL over at least a portion of the pH range of 1-8.

Water-soluble polymers suitable for use with the compounds of Formula I or Formula II may be cellulosic or non-cellulosic. The polymers may be neutral or ionizable in aqueous solution. Of these, ionizable and cellulosic polymers are preferred, with ionizable cellulosic polymers being more preferred.

Exemplary water-soluble polymers include hydroxypropyl methyl cellulose acetate succinate (HPMCAS), hydroxypropyl methyl cellulose (HPMC), hydroxypropyl methyl cellulose phthalate (HPMCP), carboxy methyl ethyl cellulose (CMEC), cellulose acetate phthalate (CAP), cellulose acetate trimellitate (CAT), polyvinylpyrrolidone (PVP), hydroxypropyl cellulose (HPC), methyl cellulose (MC), block copolymers of ethylene oxide and propylene oxide (PEO/PPO, also known as poloxamers), and mixtures thereof. Especially preferred polymers include HPMCAS, HPMC, HPMCP, CMEC, CAP, CAT, PVP, poloxamers, and mixtures thereof. Most preferred is HPMCAS. See European Patent Application Publication No. 0 901 786 A2, the disclosure of which is incorporated herein by reference.

The solid amorphous dispersions may be prepared according to any process for forming solid amorphous dispersions that results in at least a major portion (at least 60% by weight) of the poorly soluble compound being in the amorphous state. Such processes include mechanical, thermal and solvent processes. Exemplary mechanical processes include milling and extrusion; melt processes including high temperature fusion, solvent-modified fusion and melt-congeal processes; and solvent processes including non-solvent precipitation, spray coating and spray drying. See, for example, the following U.S. patents, the pertinent disclosures of which are incorporated herein by reference: U.S. Pat. Nos. 5,456,923 and 5,939,099, which describe forming dispersions by extrusion processes; U.S. Pat. Nos. 5,340,591 and 4,673,564, which describe forming dispersions by milling processes; and U.S. Pat. Nos. 5,707,646 and 4,894,235, which describe forming dispersions by melt congeal processes. In a preferred process, the solid amorphous dispersion is formed by spray drying, as disclosed in European Patent Application Publication No. 0 901 786 A2. In this process, the compound and polymer are dissolved in a solvent, such as acetone or methanol, and the solvent is then rapidly removed from the solution by spray drying to form the solid amorphous dispersion. The solid amorphous dispersions may be prepared to contain up to about 99 wt. % of the compound, e.g., 1 wt. %, 5 wt. %, 10 wt. %, 25 wt. %, 50 wt. %, 75 wt. %, 95 wt. %, or 98 wt. % as desired.

The solid dispersion may be used as the dosage form itself or it may serve as a manufacturing-use-product (MUP) in the preparation of other dosage forms such as capsules, tablets, solutions or suspensions. An example of an aqueous suspension is an aqueous suspension of a 1:1 (w/w) compound/HPMCAS-HF spray-dried dispersion containing 2.5 mg/mL of compound in 2% polysorbate-80. Solid dispersions for use in a tablet or capsule will generally be mixed with other excipients or adjuvants typically found in such dosage forms. For example, an exemplary filler for capsules contains a 2:1 (w/w) compound/HPMCAS-MF spray-dried dispersion (60%), lactose (fast flow) (15%), microcrystalline cellulose (e.g., Avicel.sup.(R0-102) (15.8%), sodium starch (7%), sodium lauryl sulfate (2%) and magnesium stearate (1%).

The HPMCAS polymers are available in low, medium and high grades as Aqoat.sup.(R)-LF, Aqoat.sup.(R)-MF and Aqoat.sup.(R)-HF respectively from Shin-Etsu Chemical Co., LTD, Tokyo, Japan. The higher MF and HF grades are generally preferred.

The compound of Formula I or Formula II or a pharmaceutically acceptable salt of said compound can be used for treating non-human animals. The administration of the compounds of Formula I or Formula II and combinations with another effective agent used to treat the relevant condition can be effected orally or non-orally.

An amount of a compound of Formula I or Formula II or combination of a compound of Formula I or Formula II with another effective agent is administered such that an effective dose is received. Generally, a daily dose that is administered orally to an animal is between about 0.01 and about 1,000 mg/kg of body weight, e.g., between about 0.01 and about 300 mg/kg or between about 0.01 and about 100 mg/kg or between about 0.01 and about 50 mg/kg of body weight, or between about 0.01 and about 25 mg/kg, or about 0.01 and about 10 mg/kg or about 0.01 and about 5 mg/kg.

Conveniently, a compound of Formula I or Formula II (or combination) can be carried in the drinking water so that a therapeutic dosage of the compound is ingested with the daily water supply. The compound can be directly metered into drinking water, preferably in the form of a liquid, water-soluble concentrate (such as an aqueous solution of a water-soluble salt).

Conveniently, a compound of Formula I or Formula II (or combination) can also be added directly to the feed, as such, or in the form of an animal feed supplement, also referred to as a premix or concentrate. A premix or concentrate of the compound in an excipient, diluent or carrier is more commonly employed for the inclusion of the agent in the feed. Suitable excipients, diluents or carriers are liquid or solid, as desired, such as water, various meals such as alfalfa meal, soybean meal, cottonseed oil meal, linseed oil meal, corncob meal and corn meal, molasses, urea, bone meal, and mineral mixes such as are commonly employed in poultry feeds. A particularly effective excipient, diluent or carrier is the respective animal feed itself; that is, a small portion of such feed. The carrier facilitates uniform distribution of the compound in the finished feed with which the premix is blended. Preferably, the compound is thoroughly blended into the premix and, subsequently, the feed. In this respect, the compound may be dispersed or dissolved in a suitable oily vehicle such as soybean oil, corn oil, cottonseed oil, and the like, or in a volatile organic solvent and then blended with the carrier. It will be appreciated that the proportions of compound in the concentrate are capable of wide variation since the amount of the compound in the finished feed may be adjusted by blending the appropriate proportion of premix with the feed to obtain a desired level of compound.

High potency concentrates may be blended by the feed manufacturer with proteinaceous carrier such as soybean oil meal and other meals, as described above, to produce concentrated supplements, which are suitable for direct feeding to animals. In such instances, the animals are permitted to consume the usual diet. Alternatively, such concentrated supplements may be added directly to the feed to produce a nutritionally balanced, finished feed containing a therapeutically effective level of a compound. The mixtures are thoroughly blended by standard procedures, such as in a twin shell blender, to ensure homogeneity.

If the supplement is used as a top dressing for the feed, it likewise helps to ensure uniformity of distribution of the compound across the top of the dressed feed.

Drinking water and feed effective for increasing lean meat deposition and for improving lean meat to fat ratio are generally prepared by mixing a compound of Formula I or Formula II with a sufficient amount of animal feed to provide from about 0.001 to about 500 ppm of the compound in the feed or water.

The preferred medicated swine, cattle, sheep and goat feed generally contain from about 1 to about 400 grams of a compound of Formula I or Formula II (or combination) per ton of feed, the optimum amount for these animals usually being about 50 to about 300 grams per ton of feed.

The preferred poultry and domestic pet feeds usually contain about 1 to about 400 grams and preferably about 10 to about 400 grams of a compound (or combination) per ton of feed.

For parenteral administration in animals, the compounds of Formula I or Formula II (or combination) may be prepared in the form of a paste or a pellet and administered as an implant, usually under the skin of the head or ear of the animal in which increase in lean meat deposition and improvement in lean meat to fat ratio is sought.

Paste formulations may be prepared by dispersing the drug in a pharmaceutically acceptable oil such as peanut oil, sesame oil, corn oil or the like.

Pellets containing an effective amount of a compound of Formula I or Formula II, pharmaceutical composition, or combination may be prepared by admixing a compound of Formula I or Formula II or combination with a diluent such as carbowax, carnauba wax, and the like, and a lubricant, such as magnesium or calcium stearate, may be added to improve the pelleting process.

It is, of course, recognized that more than one pellet may be administered to an animal to achieve the desired dose level which will provide the increase in lean meat deposition and improvement in lean meat to fat ratio desired. Moreover, implants may also be made periodically during the animal treatment period in order to maintain the proper drug level in the animal's body.

Liposomes containing these agents and/or compounds of the invention are prepared by methods known in the art, such as described in U.S. Pat. Nos. 4,485,045 and 4,544,545.

Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

These agents and/or the compounds of the invention may also be entrapped in microcapsules prepared, for example, by coacervation techniques or by interfacial polymerization, for example, hydroxymethylcellulose or gelatin-microcapsules and polymethylmethacrylate microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

The formulations to be used for intravenous administration must be sterile. This is readily accomplished by, for example, filtration through sterile filtration membranes. Compounds of the invention are generally placed into a container having a sterile access port, for example, an intravenous solution bag or vial having a stopper pierceable by a hypodermic injection needle.

Suitable emulsions may be prepared using commercially available fat emulsions, such as Intralipid®, Liposyn®, Infonutrol™, Lipofundin® and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing a compound of the invention with Intralipid™ or the components thereof (soybean oil, egg phospholipids, glycerol and water).

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device, or the nebulizing device may be attached to a face mask, tent or intermittent positive pressure breathing machine. Solution, suspension or powder compositions may be administered, preferably orally or nasally, from devices which deliver the formulation in an appropriate manner.

The compounds herein may be formulated for oral, buccal, intranasal, parenteral (e.g., intravenous, intramuscular or subcutaneous) or rectal administration or in a form suitable for administration by inhalation. The compounds of the invention may also be formulated for sustained delivery.

Methods of preparing various pharmaceutical compositions with a certain amount of active ingredient are known, or will be apparent in light of this disclosure, to those skilled in this art. For examples of methods of preparing pharmaceutical compositions see Remington's Pharmaceutical Sciences, 20th Edition (Lippincott Williams & Wilkins, 2000).

Pharmaceutical compositions according to the invention may contain 0.1%-95% by weight of the compound(s) of this invention, preferably 1%-70%. In any event, the composition to be administered will contain a quantity of a compound(s) according to the invention in an amount effective to treat the disease/condition of the subject being treated.

Since this application has an aspect that relates to the treatment of the disease/conditions described herein with a combination of active ingredients which may be administered separately, the invention also relates to combining separate pharmaceutical compositions in kit form. The kit can comprise a composition that includes a compound of the Formula I or Formula II or it can contain at least two separate pharmaceutical compositions: a compound of Formula I or Formula II, a prodrug thereof, or a salt of such compound or prodrug and a second compound as described above. The kit comprises a means for containing the separate compositions such as a container, a divided bottle or a divided foil packet. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of Formula I or Formula II compound can consist of one tablet or capsule while a daily dose of an optional second compound can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

Also, as this application has an aspect that relates to the treatment of the disease/conditions described herein with a combination of active ingredients which may be administered jointly, the invention also relates to combining separate pharmaceutical compositions in a single dosage form, such as (but not limited to) a single tablet or capsule, a bilayer or multilayer tablet or capsule, or through the use of segregated components or compartments within a tablet or capsule.

The active ingredient may be delivered as a solution in an aqueous or non-aqueous vehicle, with or without additional solvents, co-solvents, excipients, or complexation agents selected from pharmaceutically acceptable diluents, excipients, vehicles, or carriers.

The active ingredient may be formulated as a solid dispersion or as a self-emulsified drug delivery system (SEDDS) with pharmaceutically acceptable excipients.

The active ingredient may be formulated as an immediate release or modified release tablet or capsule. Alternatively, the active ingredient may be delivered as the active ingredient alone within a capsule shell, without additional excipients.

### Experimental Procedures

The following illustrate the synthesis of various compounds of the present invention. Additional compounds within the scope of this invention may be prepared using the methods illustrated in these Examples, either alone or in combination with techniques generally known in the art. All starting materials in these Preparations and Examples are either commercially available or can be prepared by methods known in the art or as described herein.

Reactions were performed in air or, when oxygen- or moisture-sensitive reagents or intermediates were employed, under an inert atmosphere (nitrogen or argon). When appropriate, reaction apparatuses were dried under dynamic vacuum using a heat gun, and anhydrous solvents (Sure-Seal™ products from Aldrich Chemical Company, Milwaukee, Wisconsin or DriSolv™ products from EMD Chemicals, Gibbstown, NJ) were employed. In some cases, commercial solvents were passed through columns packed with 4 Å molecular sieves, until the following QC standards for water were attained: a) <100 ppm by weight for dichloromethane, toluene, N,N-dimethylformamide, and tetrahydrofuran; b) <180 ppm for methanol, ethanol, 1,4-dioxane, and diisopropylamine. For very sensitive reactions, solvents were further treated with metallic sodium, calcium hydride, or molecular sieves, and distilled just prior to use. Other commercial solvents and reagents were used without further purification. For syntheses referencing procedures in other Examples or Methods, reaction conditions (reaction time and temperature) may vary. Products were generally dried under vacuum before being carried on to further reactions or submitted for biological testing.

When indicated, reactions were heated by microwave irradiation using Biotage Initiator or Personal Chemistry Emrys Optimizer microwave instruments. Reaction progress was monitored using thin-layer chromatography (TLC), liquid chromatography-mass spectrometry (LCMS), high-performance liquid chromatography (HPLC), and/or gas chromatography-mass spectrometry (GCMS) analyses. TLC was performed on pre-coated silica gel plates with a fluorescence indicator (254 nm excitation wavelength) and visualized under UV light and/or with I₂, KMnO₄, CoCl₂, phosphomolybdic acid, or ceric ammonium molybdate stains. LCMS data were acquired on an Agilent 1100 Series instrument with a Leap Technologies autosampler, Gemini C18 columns, acetonitrile/water gradients, and either trifluoroacetic acid, formic acid, or ammonium hydroxide modifiers. The column eluent was analyzed using a Waters ZQ mass spectrometer scanning in both positive and negative ion modes from 100 to 1200 Da. Other similar instruments were also used. HPLC data were generally acquired on an Agilent 1100 Series instrument using Gemini or XBridge C18 columns, acetonitrile/water gradients, and either trifluoroacetic acid or ammonium hydroxide modifiers. GCMS data were acquired using a Hewlett Packard 6890 oven with an HP 6890 injector, HP-1 column (12 m×0.2 mm×0.33 μm), and helium carrier gas. Samples were analyzed on an HP 5973 mass selective detector, scanning from 50 to 550 Da using electron ionization. Purifications were performed by medium performance liquid chromatography (MPLC) using Isco CombiFlash Companion, AnaLogix IntelliFlash 280, Biotage SP1, or Biotage Isolera One instruments and pre-packed Isco RediSep or Biotage Snap silica cartridges. Chiral purifications were generally performed by chiral supercritical fluid chromatography (SFC) using Berger or Thar instruments; ChiralPAK-AD, -AS, -IC, Chiralcel-OD, or -OJ columns; and CO₂ mixtures with methanol, ethanol, propan-2-ol, or acetonitrile, alone or modified using trifluoroacetic acid or propan-2-amine. UV detection was used to trigger fraction collection. For syntheses referencing procedures in other Examples or Methods, purifications may vary: in general, solvents and the solvent ratios used for eluents/gradients were chosen to provide appropriate R_{f}s or retention times.

Mass spectrometry data are reported from LCMS analyses. Mass spectrometry (MS) was performed via atmospheric pressure chemical ionization (APCI), electrospray ionization (ESI), electron impact ionization (EI) or electron scatter (ES) ionization sources. Proton nuclear magnetic spectroscopy (¹H NMR) chemical shifts are given in parts per million downfield from tetramethylsilane and were recorded on 300, 400, 500, or 600 MHz Varian, Bruker, or Jeol spectrometers. Chemical shifts are expressed in parts per million (ppm, d) referenced to the deuterated solvent residual peaks (chloroform, 7.26 ppm; CD₂HOD, 3.31 ppm; acetonitrile-d₂, 1.94 ppm; dimethyl sulfoxide-d₅, 2.50 ppm; DHO, 4.79 ppm). The peak shapes are described as follows: s, singlet; d, doublet; t, triplet; q, quartet; quin, quintet; m, multiplet; br s, broad singlet; app, apparent. Analytical SFC data were acquired on a Berger analytical instrument as described above. Optical rotation data were acquired on a PerkinElmer model 343 polarimeter using a 1 dm cell. Silica gel chromatography was performed primarily using medium-pressure Biotage or ISCO systems using columns pre-packaged by various commercial vendors including Biotage and ISCO. Microanalyses were performed by Quantitative Technologies Inc. and were within 0.4% of the calculated values.

Unless otherwise noted, chemical reactions were performed at room temperature (about 23 degrees Celsius).

Unless noted otherwise, all reactants were obtained commercially without further purifications or were prepared using methods known in the literature.

Hydrogenation may be performed in a Parr Shaker under pressurized hydrogen gas, or in a Thales-nano H-Cube flow hydrogenation apparatus at full hydrogen and a flow rate between 1 and 2 mL/minute at the specified temperature.

HPLC, UPLC, LCMS, GCMS, and SFC retention times were measured using the methods noted in the procedures.

In some examples, chiral separations were carried out to separate enantiomers or diastereomers of certain compounds of the invention (in some examples, the separated enantiomers are designated as ENANT-1 and ENANT-2, according to their order of elution; similarly, separated diastereomers are designated as DIAST-1 and DIAST-2, according to their order of elution). In some examples, the optical rotation of an enantiomer was measured using a polarimeter. According to its observed rotation data (or its specific rotation data), an enantiomer with a clockwise rotation was designated as the (+)-enantiomer and an enantiomer with a counter-clockwise rotation was designated as the (−)-enantiomer. Racemic compounds are indicated either by the absence of drawn or described stereochemistry, or by the presence of (+/−) adjacent to the structure; in this latter case, the indicated stereochemistry represents just one of the two enantiomers that make up the racemic mixture.

The compounds and intermediates described below were named using the naming convention provided with ACD/ChemSketch 2017.2.1, File Version C40H41, Build 99535 (Advanced Chemistry Development, Inc., Toronto, Ontario, Canada). The naming convention provided with ACD/ChemSketch 2017.2.1 is well known by those skilled in the art and it is believed that the naming convention provided with ACD/ChemSketch 2017.2.1 generally comports with the IUPAC (International Union for Pure and Applied Chemistry) recommendations on Nomenclature of Organic Chemistry and the CAS Index rules.

### EXAMPLES

### Preparation P1

### 3,5-Difluoro-4-[(4-methoxyphenyl)methoxy]benzoic acid (P1)

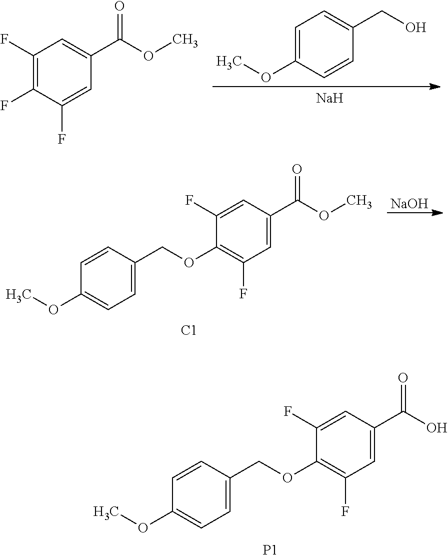

Step 1. Synthesis of methyl 3,5-difluoro-4-[(4-methoxyphenyl)methoxy]benzoate (C1): To a 0° C. solution of sodium hydride (60% dispersion in mineral oil; 1.60 g, 40.0 mmol) in tetrahydrofuran (200 mL) was added (4-methoxyphenyl)methanol (5.25 g, 38.0 mmol). After the reaction mixture had been stirred at 0° C. for 30 minutes, a solution of methyl 3,4,5-trifluorobenzoate (7.00 g, 36.8 mmol) in tetrahydrofuran (50 mL) was added, whereupon the reaction mixture was allowed to warm to 25° C. and stir for 1 hour. It was then quenched by addition of saturated aqueous ammonium chloride solution, and the aqueous layer was extracted with ethyl acetate; the combined organic layers were dried over sodium sulfate, filtered, and concentrated in vacuo to provide C1 as a solid (11.2 g). This material was progressed directly to the following step.

Step 2. Synthesis of 3,5-difluoro-4-[(4-methoxyphenyl)methoxy]benzoic acid (P1): To a solution of C1 (from the previous step; 11.2 g, 536.3 mmol) in methanol (200 mL) was added a solution of sodium hydroxide (4.36 g, 109 mmol) in water (20 mL), whereupon the reaction mixture was stirred at 26° C. for 4 hours. It was then concentrated in vacuo, and the aqueous residue was washed with dichloromethane (2×150 mL). After the aqueous layer had been acidified to pH 5, it was extracted with dichloromethane (3×300 mL), and these three dichloromethane layers were combined and washed with saturated aqueous sodium chloride solution, dried over sodium sulfate, filtered, and concentrated under reduced pressure to afford P1 as a white solid. Yield: 10 g, 34 mmol, 92% over 2 steps. ¹H NMR (400 MHz, DMSO-d₆) δ 7.61-7.53 (m, 2H), 7.34 (d, J=8.6 Hz, 2H), 6.92 (d, J=8.7 Hz, 2H), 5.20 (s, 2H), 3.74 (s, 3H).

### Preparation P2

### 2,3,5-Trifluoro-4-[(4-methoxyphenyl)methoxy]benzoic acid (P2)

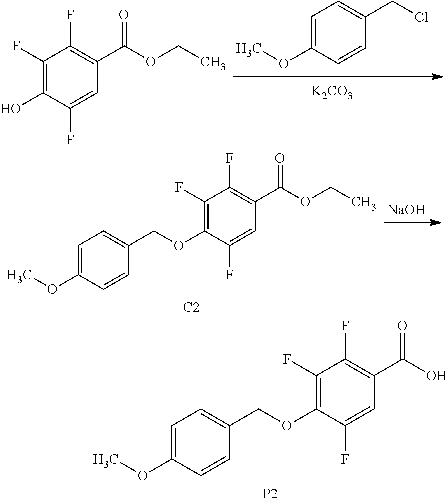

Step 1. Synthesis of ethyl 2,3,5-trifluoro-4-[(4-methoxyphenyl)methoxy]benzoate (C2): 1-(Chloromethyl)-4-methoxybenzene (40.1 g, 256 mmol) was added to a mixture of ethyl 2,3,5-trifluoro-4-hydroxybenzoate (51.3 g, 233 mmol) and potassium carbonate (64.3 g, 465 mmol) in acetonitrile (100 mL). After the reaction mixture had been stirred at 80° C. for 16 hours, LCMS analysis indicated conversion to C2: LCMS m/z 363.1 [M+Na⁺]. Solids were removed via filtration, and the filtrate was concentrated in vacuo to provide C2 as a yellow oil. Yield: 71.0 g, 209 mmol, 90%.

Step 2. Synthesis of 2,3,5-trifluoro-4-[(4-methoxyphenyl)methoxy]benzoic acid (P2): To a solution of C2 (71.0 g, 209 mmol) in methanol (500 mL) was added an aqueous solution of sodium hydroxide (3 M; 300 mL). After the reaction mixture had been stirred at 50° C. for 4 hours, it was concentrated in vacuo. The aqueous residue was acidified by addition of 1 M hydrochloric acid, and the resulting solid was collected via filtration to afford P2 as a white solid. Yield: 51.7 g, 166 mmol, 79%. LCMS m/z 335.1 [M+Na⁺]. ¹H NMR (400 MHz, DMSO-d₆) δ 7.38-7.29 (m, 1H), 7.34 (d, J=8.6 Hz, 2H), 6.93 (d, J=8.7 Hz, 2H), 5.18 (s, 2H), 3.75 (s, 3H).

### Preparation P3

### N-{[(1r,4r)-4-Aminocyclohexyl]methyl}-3,5-difluoro-4-[(4-methoxyphenyl)methoxy]benzamide (P3)

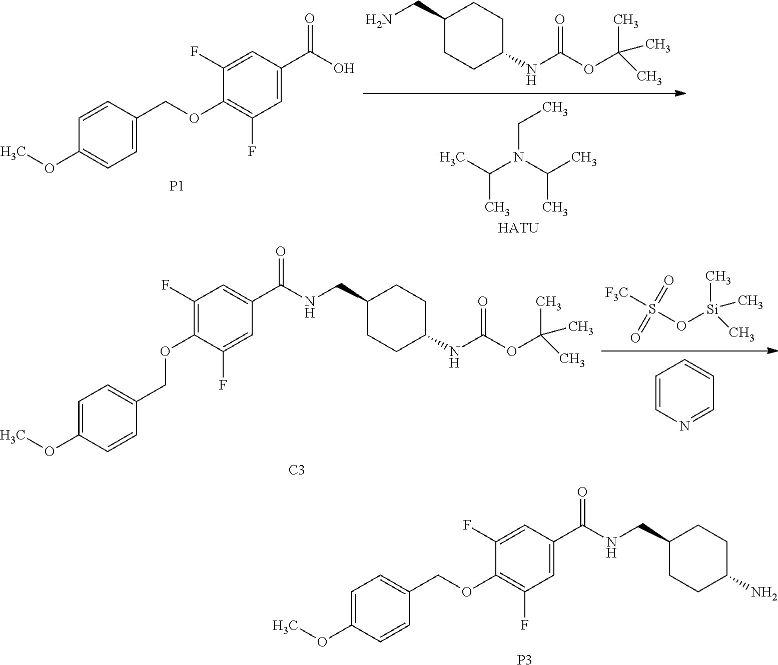

Step 1. Synthesis of tert-butyl [(1r,4r)-4-({3,5-difluoro-4-[(4-methoxyphenyl)methoxy]benzamido}methyl)cyclohexyl]carbamate (C3): To a solution of P1 (19.3 g, 65.6 mmol), N,N-diisopropylethylamine (25.4 g, 197 mmol), and O-(7-azabenzotriazol-1-yl)-N,N,N′,N′-tetramethyluronium hexafluorophosphate (HATU; 27.5 g, 72.3 mmol) in dichloromethane (700 mL) was added tert-butyl [(1r,4r)-4-(aminomethyl)cyclohexyl]carbamate (15.0 g, 65.7 mmol).

After the reaction mixture had been stirred at 25° C. for 16 hours, LCMS analysis indicated the presence of C3: LCMS m/z 527.3 [M+Na⁺]. Filtration was followed by washing of the filter cake with water and with a mixture of dichloromethane and ethyl acetate, affording C₃ as a white solid. Yield: 26.5 g, 52.5 mmol, 80%. ¹H NMR (400 MHz, DMSO-d₆) δ 8.48 (br t, J=6 Hz, 1H), 7.63-7.53 (m, 2H), 7.33 (d, J=8.6 Hz, 2H), 6.92 (d, J=8.7 Hz, 2H), 6.67 (br d, J=8.0 Hz, 1H), 5.16 (s, 2H), 3.74 (s, 3H), 3.22-3.10 (m, 1H), 3.06 (dd, J=6.1, 6.1 Hz, 2H), 1.83-1.65 (m, 4H), 1.48-1.36 (m, 1H), 1.36 (s, 9H), 1.16-1.02 (m, 2H), 1.00-0.85 (m, 2H).

Step 2. Synthesis of N-{[(1r,4r)-4-aminocyclohexyl]methyl}-3,5-difluoro-4-[(4-methoxyphenyl)methoxy]benzamide (P3): To a 0° C. solution of C3 (21.5 g, 42.6 mmol) and pyridine (27.0 g, 341 mmol) in dichloromethane (500 mL) was added trimethylsilyl trifluoromethanesulfonate (37.9 g, 170 mmol) in a drop-wise manner. After the reaction mixture had been stirred at 25° C. for 16 hours, aqueous sodium bicarbonate solution (100 mL) was added, and the mixture was filtered. The filter cake was washed with water and with a mixture of dichloromethane and ethyl acetate, providing P3 as a white solid. Yield: 10.0 g, 24.7 mmol, 58%. LCMS m/z 405.3 [M+H]⁺. ¹H NMR (400 MHz, DMSO-d₆) δ 8.50 (br t, J=6 Hz, 1H), 7.71-7.41 (m, 4H), 7.33 (d, J=8.2 Hz, 2H), 6.92 (d, J=8.2 Hz, 2H), 5.17 (s, 2H), 3.74 (s, 3H), 3.09 (dd, J=6 Hz, 2H), 3.00-2.86 (m, 1H), 1.96-1.85 (m, 2H), 1.82-1.70 (m, 2H), 1.54-1.38 (m, 1H), 1.31-1.15 (m, 2H), 1.07-0.92 (m, 2H).

### Preparation P4

### (1r,4r)-4-({3,5-Difluoro-4-[(4-methoxyphenyl)methoxy]benzamido}methyl)cyclohexane-1-carboxylic acid (P4)

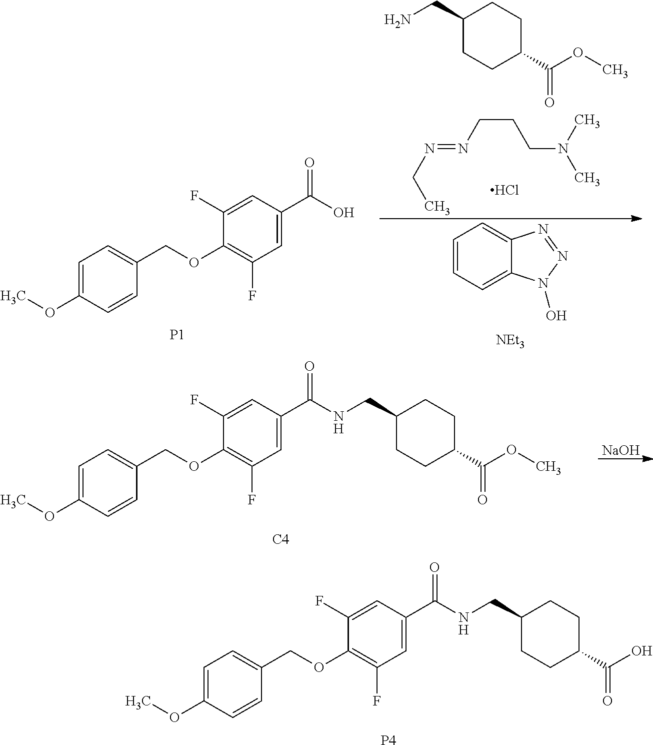

Step 1. Synthesis of methyl (1r,4r)-4-({3,5-difluoro-4-[(4-methoxyphenyl)methoxy]benzamido}methyl)cyclohexane-1-carboxylate (C4): To a solution of P1 (18.0 g, 61.2 mmol), 1-[3-(dimethylamino)propyl]-3-ethylcarbodiimide hydrochloride (14.1 g, 73.5 mmol), and 1H-benzotriazol-1-ol (9.92 g, 73.4 mmol) in dichloromethane (500 mL) were added triethylamine (7.41 g, 73.2 mmol) and methyl (1r,4r)-4-(aminomethyl)cyclohexane-1-carboxylate (10.5 g, 61.3 mmol). After the reaction mixture had been stirred at 28° C. for 4 hours, it was extracted with dichloromethane. The combined organic layers were washed with saturated aqueous sodium chloride solution, dried over sodium sulfate, filtered, concentrated in vacuo, and purified via silica gel chromatography (Eluent: 6% methanol in dichloromethane), providing C4 as a white solid. Yield: 22.0 g, 49.2 mmol, 80%. LCMS m/z 448.2 [M+H]⁺.

Step 2. Synthesis of (1r,4r)-4-({3,5-difluoro-4-[(4-methoxyphenyl)methoxy]benzamido}methyl)cyclohexane-1-carboxylic acid (P4): A solution of sodium hydroxide (8.05 g, 201 mmol) in water (20 mL) was added to a solution of C4 (18.0 g, 40.2 mmol) in methanol (200 mL). The reaction mixture was stirred at 26° C. for 6 hours, whereupon methanol was removed under reduced pressure and the aqueous residue was washed with dichloromethane (2×20 mL). The aqueous layer was then adjusted to pH 5 and extracted with dichloromethane (3×50 mL); these three extracts were combined, washed with saturated aqueous sodium chloride solution, dried over sodium sulfate, filtered, and concentrated in vacuo to afford P4 as a white solid. Yield: 14.0 g, 32.3 mmol, 80%. LCMS m/z 434.2 [M+H]⁺. ¹H NMR (400 MHz, DMSO-d₆) δ 8.50 (br t, J=5.7 Hz, 1H), 7.64-7.54 (m, 2H), 7.33 (d, J=8.5 Hz, 2H), 6.92 (d, J=8.4 Hz, 2H), 5.16 (s, 2H), 3.74 (s, 3H), 3.08 (dd, J=6, 6 Hz, 2H), 2.18-2.05 (m, 1H), 1.95-1.82 (m, 2H), 1.80-1.68 (m, 2H), 1.54-1.39 (m, 1H), 1.33-1.16 (m, 2H), 1.02-0.85 (m, 2H).

### Preparation P5

### 3,5-Difluoro-N-{[(1r,4r)-4-(N-hydroxycarbamimidoyl)cyclohexyl]methyl}-4-[(4-methoxyphenyl)methoxy]benzamide (P5)

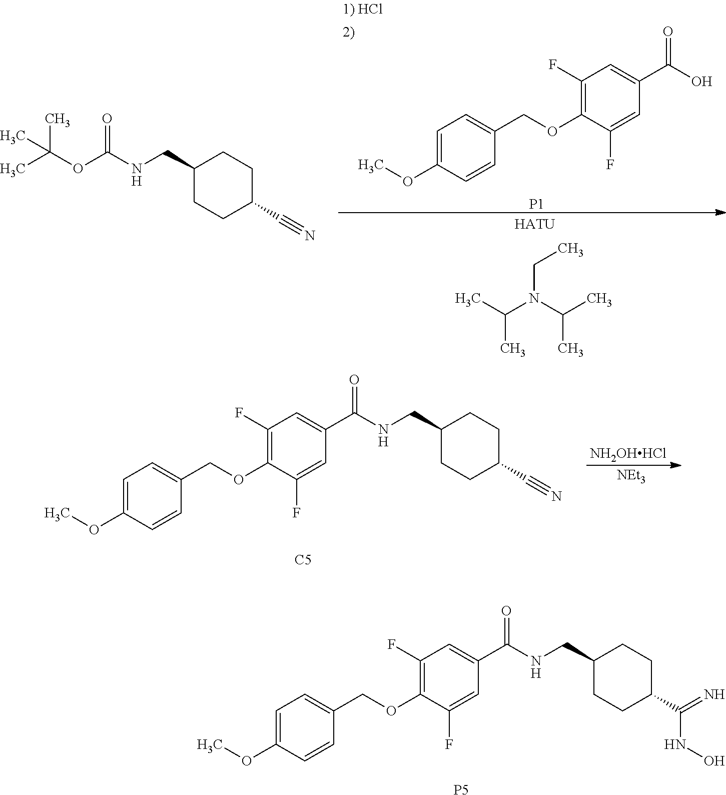

Step 1. Synthesis of N-{[(1r,4r)-4-cyanocyclohexyl]methyl}-3,5-difluoro-4-[(4-methoxyphenyl)methoxy]benzamide (C5): A solution of hydrogen chloride in 1,4-dioxane (4 M; 50 mL, 200 mmol) was added to a solution of tert-butyl {[(1r,4r)-4-cyanocyclohexyl]methyl}carbamate (4.86 g, 20.4 mmol) in tetrahydrofuran (50 mL), and the mixture was stirred at room temperature overnight. After removal of solvents via concentration under reduced pressure, the residue was triturated with diethyl ether to provide (1r,4r)-4-(aminomethyl)cyclohexane-1-carbonitrile, hydrochloride salt.

O-(7-Azabenzotriazol-1-yl)-N,N,N′N′-tetramethyluronium hexafluorophosphate (HATU; 95%, 8.16 g, 20.4 mmol) was added to a solution of P1 (5.0 g, 17 mmol) in dichloromethane (113 mL). After this mixture had been stirred for 1 hour, it was treated with N,N-diisopropylethylamine (8.88 mL, 51.0 mmol) and the (1r,4r)-4-(aminomethyl)cyclohexane-1-carbonitrile, hydrochloride salt from above. The reaction mixture was stirred at room temperature for 3 days, whereupon it was washed sequentially with water, 1 M hydrochloric acid, water, saturated aqueous sodium bicarbonate solution, and saturated aqueous sodium chloride solution, dried over sodium sulfate, filtered, and concentrated in vacuo. The residue was dissolved in a minimal quantity of a hot 10:1 mixture of ethyl acetate and heptane; after cooling to room temperature, this was filtered, and the filtrate was concentrated under reduced pressure. Silica gel chromatography provided C5 as a white solid. Yield: 5.80 g, 14.0 mmol, 82%. LCMS m/z 415.3 [M+H]⁺. ¹H NMR (400 MHz, DMSO-d₆) δ 8.50 (br t, J=5.8 Hz, 1H), 7.62-7.53 (m, 2H), 7.33 (d, J=8.6 Hz, 2H), 6.92 (d, J=8.6 Hz, 2H), 5.16 (s, 2H), 3.74 (s, 3H), 3.08 (dd, J=6, 6 Hz, 2H), 2.62 (tt, J=11.9, 3.6 Hz, 1H), 2.04-1.95 (m, 2H), 1.77-1.67 (m, 2H), 1.60-1.37 (m, 3H), 1.04-0.89 (m, 2H).

Step 2. Synthesis of 3,5-difluoro-N-{[(1r,4r)-4-(N-hydroxycarbamimidoyl)cyclohexyl]methyl}-4-[(4-methoxyphenyl)methoxy]benzamide (P5): Hydroxylamine hydrochloride (8.38 g, 121 mmol) and triethylamine (16.8 mL, 121 mmol) were added to a solution of C5 (5.00 g, 12.1 mmol) in methanol (50 mL). The reaction mixture was heated at 50° C. for 24 hours, whereupon it was cooled to room temperature and concentrated in vacuo. The residue was partitioned between water (100 mL) and ethyl acetate (100 mL) and the mixture was vigorously stirred for 15 minutes. Filtration, followed by rinsing of the collected solids with water (50 mL) and with ethyl acetate (50 mL) provided P5 as a white solid. Yield: 4.50 g, 10.1 mmol, 83%. LCMS m/z 448.4 [M+H]⁺. ¹H NMR (400 MHz, DMSO-d₆), characteristic peaks: δ 9.42 (s, 1H), 8.47 (br t, J=5.8 Hz, 1H), 8.19 (br s, 1H), 7.81 (br s, 1H), 7.63-7.54 (m, 2H), 7.33 (d, J=8.6 Hz, 2H), 6.92 (d, J=8.7 Hz, 2H), 5.16 (s, 2H), 3.74 (s, 3H), 3.09 (dd, J=6, 6 Hz, 2H), 2.5-2.40 (m, 1H, assumed; partially obscured by solvent peak), 1.96-1.84 (m, 2H), 1.56-1.41 (m, 1H), 1.02-0.87 (m, 2H).

### Preparation P6

### N-{[(1s,4s)-4-Bromocyclohexyl]methyl}-3,5-difluoro-4-[(4-methoxyphenyl)methoxy]benzamide (P6)

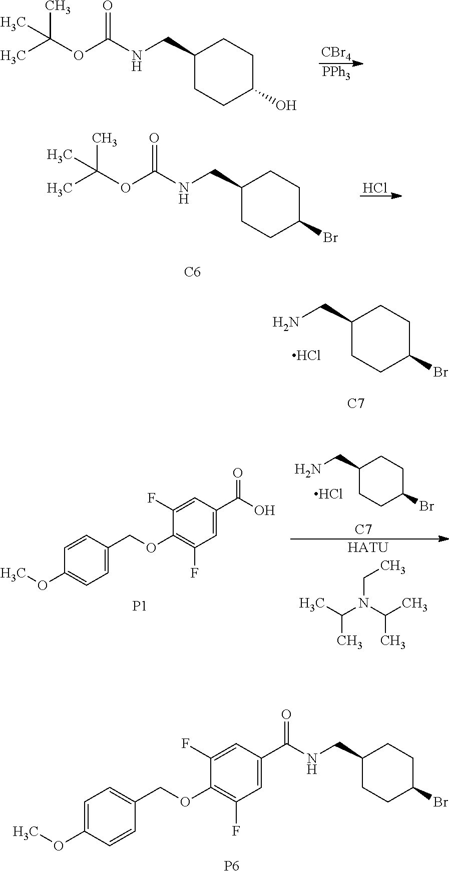

Step 1. Synthesis of tert-butyl {[(1s,4s)-4-bromocyclohexyl]methyl}carbamate (C6): To a 0° C. solution of tert-butyl {[(1r,4r)-4-hydroxycyclohexyl]methyl}carbamate (5.00 g, 21.8 mmol) in dichloromethane (150 mL) was added carbon tetrabromide (10.8 g, 32.6 mmol). Triphenylphosphine (8.58 g, 32.7 mmol) was added portion-wise, and the reaction mixture was stirred at 25° C. for 48 hours. After removal of solvent in vacuo, purification via silica gel chromatography (Gradient: 0% to 20% ethyl acetate in petroleum ether) afforded C₆. Yield: 1.30 g, 4.45 mmol, 20%. LCMS m/z 314.1 (bromine isotope pattern observed) [M+Na⁺]. ¹H NMR (400 MHz, DMSO-d₆) δ 6.86 (br t, J=6.0 Hz, 1H), 4.78-4.71 (m, 1H), 2.82 (dd, J=6, 6 Hz, 2H), 1.99-1.89 (m, 2H), 1.87-1.75 (m, 2H), 1.57-1.26 (m, 5H), 1.37 (s, 9H).

Step 2. Synthesis of 1-[(1s,4s)-4-bromocyclohexyl]methanamine, hydrochloride salt (C7): To a solution of C6 (1.30 g, 4.45 mmol) in dichloromethane (20 mL) was added a solution of hydrogen chloride in 1,4-dioxane (4 M; 15 mL). After the reaction mixture had been stirred at 25° C. for 2.5 hours, LCMS analysis indicated conversion to C7: LCMS m/z 192.1 (bromine isotope pattern observed) [M+H]⁺. Removal of solvents in vacuo afforded C7 (900 mg), which was used directly in the following step.

Step 3. Synthesis of N-{[(1s,4s)-4-bromocyclohexyl]methyl}-3,5-difluoro-4-[(4-methoxyphenyl)methoxy]benzamide (P6): To a solution of P1 (1.65 g, 5.61 mmol), O-(7-azabenzotriazol-1-yl)-N,N,N′N′-tetramethyluronium hexafluorophosphate (HATU; 2.67 g, 7.02 mmol), and N,N-diisopropylethylamine (1.82 g, 14.1 mmol) in dichloromethane (80 mL) was added C₇ (from the previous step; 900 mg, 54.45 mmol), whereupon the reaction mixture was stirred at room temperature for 3 hours. After the reaction mixture had been concentrated in vacuo, chromatography on silica gel (Gradient: 0% to 30% ethyl acetate in petroleum ether) provided P6. Yield: 1.40 g, 2.99 mmol, 67% over 2 steps. LCMS m/z 490.0 (bromine isotope pattern observed [M+Na⁺]. ¹H NMR (400 MHz, DMSO-d₆) δ 8.55 (br t, J=5.8 Hz, 1H), 7.63-7.54 (m, 2H), 7.33 (d, J=8.7 Hz, 2H), 6.92 (d, J=8.6 Hz, 2H), 5.17 (s, 2H), 4.80-4.72 (m, 1H), 3.74 (s, 3H), 3.15 (dd, J=6, 6 Hz, 2H), 2.02-1.91 (m, 2H), 1.89-1.77 (m, 2H), 1.70-1.53 (m, 3H), 1.47-1.33 (m, 2H).

### Preparation P7

### 3,5-Difluoro-N-{[4-(N-hydroxycarbamimidoyl)bicyclo[2.2.2]octan-1-yl]methyl}-4-[(4-methoxyphenyl)methoxy]benzamide (P7)

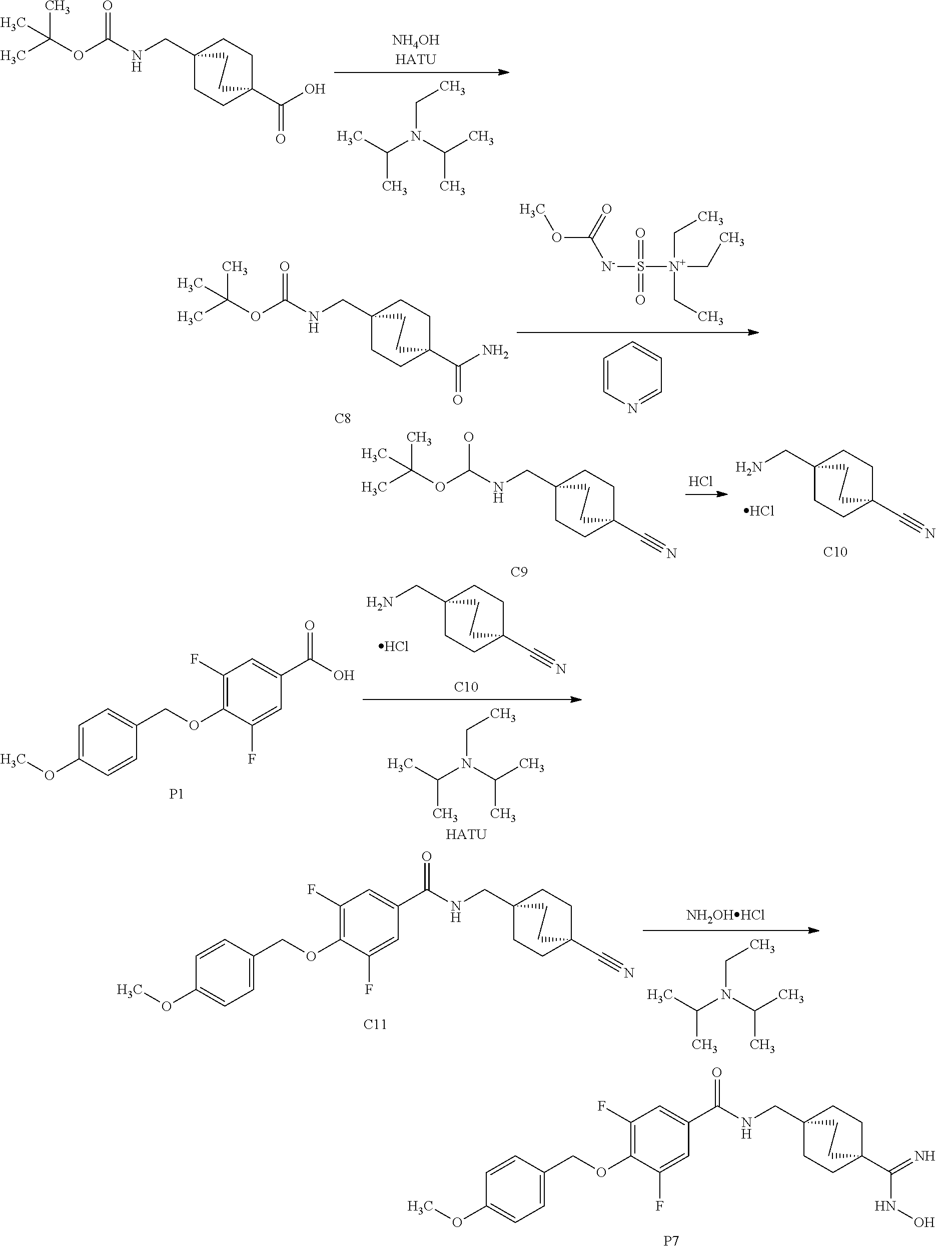

Step 1. Synthesis of tert-butyl [(4-carbamoylbicyclo[2.2.2]octan-1-yl)methyl]carbamate (C8): To a solution of 4-{[(tert-butoxycarbonyl)amino]methyl}bicyclo[2.2.2]octane-1-carboxylic acid (1.50 g, 5.29 mmol) in dichloromethane (20 mL) were added O-(7-azabenzotriazol-1-yl)-N,N,N′N′-tetramethyluronium hexafluorophosphate (HATU; 3.02 g, 7.94 mmol), N,N-diisopropylethylamine (2.05 g, 15.9 mmol), and aqueous ammonium hydroxide solution (0.3 M; 22.9 mL, 6.87 mmol). After the reaction mixture had been stirred at 25° C. for 2 hours, it was diluted with dichloromethane (25 mL), washed sequentially with water (2×20 mL) and saturated aqueous sodium chloride solution (20 mL), dried over sodium sulfate, filtered, and concentrated in vacuo. Trituration with water (20 mL) afforded C8 as a white solid. Yield: 1.20 g, 4.25 mmol, 80%. LCMS m/z 283.2 [M+H]⁺.

Step 2. Synthesis of tert-butyl [(4-cyanobicyclo[2.2.2]octan-1-yl)methyl]carbamate (C9): (Methoxycarbonylsulfamoyl)triethylammonium hydroxide, inner salt (Burgess reagent; 1.86 g, 7.81 mmol) was added to a solution of C8 (1.10 g, 3.90 mmol) in a mixture of pyridine (15 mL) and dichloromethane (10 mL). After the reaction mixture had been stirred at 25° C. for 2 hours, it was concentrated in vacuo; the residue was diluted with water (30 mL) and extracted with dichloromethane (2×20 mL). The combined organic layers were washed with saturated aqueous sodium chloride solution (2×20 mL), dried over sodium sulfate, filtered, and concentrated under reduced pressure, providing C9 as a white solid. Yield: 1.00 g, 3.78 mmol, 97%. LCMS m/z 209.2 [(M-2-methylprop-1-ene)+H]⁺. ¹H NMR (400 MHz, DMSO-d₆) 6.80 (br t, J=6.4 Hz, 1H), 2.66 (d, J=6.4 Hz, 2H), 1.86-1.76 (m, 6H), 1.36 (s, 9H), 1.36-1.27 (m, 6H).

Step 3. Synthesis of 4-(aminomethyl)bicyclo[2.2.2]octane-1-carbonitrile, hydrochloride salt (C10): To a 0° C. solution of C9 (1.00 g, 3.78 mmol) in dichloromethane (15 mL) was added a solution of hydrogen chloride in 1,4-dioxane (4 M; 3.8 mL, 15 mmol), whereupon the reaction mixture was stirred at 25° C. for 16 hours. Removal of solvents in vacuo afforded C10 as a white solid. Yield: 750 mg, 3.74 mmol, 99%. LCMS m/z 165.2 [M+H]⁺.

Step 4. Synthesis of N-[(4-cyanobicyclo[2.2.2]octan-1-yl)methyl]-3,5-difluoro-4-[(4-methoxyphenyl)methoxy]benzamide (C11): O-(7-Azabenzotriazol-1-yl)-N,N,N′N′-tetramethyluronium hexafluorophosphate (HATU; 1.60 g, 4.21 mmol) and N,N-diisopropylethylamine (1.81 g, 14.0 mmol) were added to a solution of P1 (1.13 g, 3.84 mmol) in N,N-dimethylformamide (10 mL). After the reaction mixture had been stirred at 25° C. for 10 minutes, C10 (700 mg, 3.49 mmol) was added and stirring was continued at 25° C. for 4 hours. Water (25 mL) was then added, and the resulting mixture was extracted with ethyl acetate (2×25 mL); the combined organic layers were washed sequentially with water (2×10 mL) and saturated aqueous sodium chloride solution (2×10 mL), dried over sodium sulfate, filtered, and concentrated in vacuo. Silica gel chromatography (Gradient: 0% to 50% ethyl acetate in petroleum ether) provided C11 as a light-yellow solid. Yield: 1.29 g, 2.93 mmol, 84%. LCMS m/z 441.2 [M+H]⁺. ¹H NMR (400 MHz, DMSO-d₆) δ 8.36 (br t, J=6.3 Hz, 1H), 7.64-7.54 (m, 2H), 7.34 (d, J=8.7 Hz, 2H), 6.92 (d, J=8.6 Hz, 2H), 5.17 (s, 2H), 3.75 (s, 3H), 3.01 (d, J=6.2 Hz, 2H), 1.87-1.78 (m, 6H), 1.46-1.36 (m, 6H).

Step 5. Synthesis of 3,5-difluoro-N-{[4-(N-hydroxycarbamimidoyl)bicyclo[2.2.2]octan-1-yl]methyl}-4-[(4-methoxyphenyl)methoxy]benzamide (P7): To a solution of C11 (1.20 g, 2.72 mmol) in methanol (25 mL) were added hydroxylamine hydrochloride (1.14 g, 16.4 mmol) and N,N-diisopropylethylamine (2.82 g, 21.8 mmol), whereupon the reaction mixture was stirred at 70° C. for 16 hours. Removal of solvent in vacuo provided a residue, which was purified via silica gel chromatography (Gradient: 0% to 5% methanol in dichloromethane) to afford P7 as a white solid. Yield: 748 mg, 1.58 mmol, 58%. LCMS m/z 474.2 [M+H]⁺. ¹H NMR (400 MHz, DMSO-d₆) δ 8.85 (s, 1H), 8.30 (br t, J=6.2 Hz, 1H), 7.64-7.55 (m, 2H), 7.34 (d, J=8.7 Hz, 2H), 6.92 (d, J=8.7 Hz, 2H), 5.16 (s, 2H), 5.11 (br s, 2H), 3.74 (s, 3H), 3.01 (d, J=6.2 Hz, 2H), 1.66-1.56 (m, 6H), 1.41-1.31 (m, 6H).

### Preparation P8

### N-[(4-Aminobicyclo[2.2.2]octan-1-yl)methyl]-2,3,5-trifluoro-4-[(4-methoxyphenyl)methoxy]benzamide (P8)

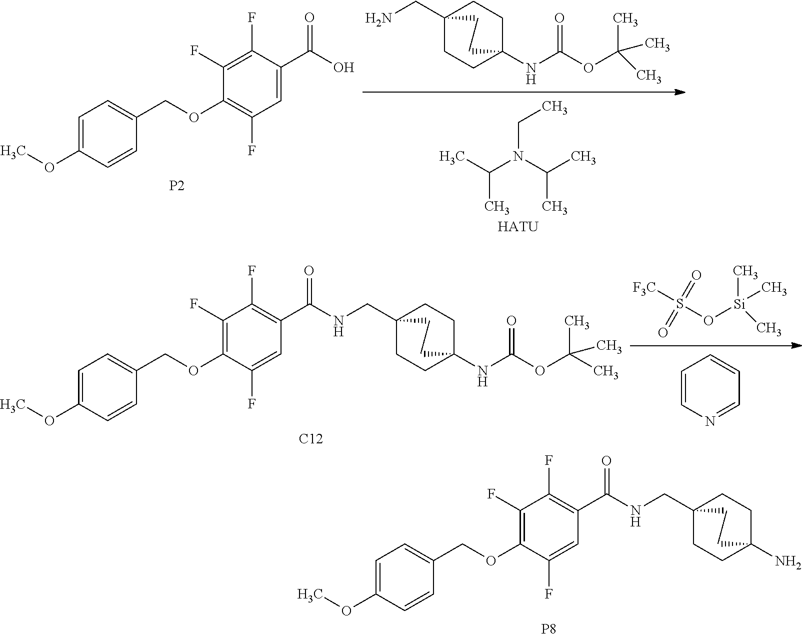

Step 1. Synthesis of tert-butyl [4-({2,3,5-trifluoro-4-[(4-methoxyphenyl)methoxy]benzamido}methyl) bicyclo[2.2.2]octan-1-yl]carbamate (C₁₂): N,N-Diisopropylethylamine (826 mg, 6.39 mmol) was added to a solution of P2 (1.00 g, 3.20 mmol) and O-(7-azabenzotriazol-1-yl)-N,N,N′N′-tetramethyluronium hexafluorophosphate (HATU; 1.46 g, 3.84 mmol) in N,N-dimethylformamide (20 mL). After the mixture had been stirred at 25° C. for 2 minutes, tert-butyl [4-(aminomethyl)bicyclo[2.2.2]octan-1-yl]carbamate (855 mg, 3.36 mmol) was added, and stirring was continued at 20° C. for 1 hour. The reaction mixture was then extracted with ethyl acetate (2×50 mL), and the combined organic layers were dried over sodium sulfate, filtered, and concentrated in vacuo. Chromatography on silica gel (Eluent: 1:1 petroleum ether/ethyl acetate) provided C12 as a white solid. Yield: 1.35 g, 2.46 mmol, 77%. LCMS m/z 549.3 [M+H]⁺. ¹H NMR (400 MHz, DMSO-d₆) δ 8.25 (t, J=6.3 Hz, 1H), 7.35 (d, J=8.6 Hz, 2H), 7.30 (ddd, J=10.9, 6.0, 2.3 Hz, 1H), 6.94 (d, J=8.7 Hz, 2H), 6.32 (br s, 1H), 5.21 (s, 2H), 3.75 (s, 3H), 2.96 (d, J=6.2 Hz, 2H), 1.76-1.64 (m, 6H), 1.46-1.37 (m, 6H), 1.35 (s, 9H).

Step 2. Synthesis of N-[(4-aminobicyclo[2.2.2]octan-1-yl)methyl]-2,3,5-trifluoro-4-[(4-methoxyphenyl)methoxy]benzamide (P8): To a solution of C12 (1.30 g, 2.37 mmol) and pyridine (1.50 g, 19.0 mmol) in dichloromethane (20 mL) was added trimethylsilyl trifluoromethanesulfonate (3.69 g, 16.6 mmol), whereupon the reaction mixture was stirred at 20° C. for 30 minutes. Aqueous sodium bicarbonate solution (2 M; 50 mL) was then added, and the resulting mixture was extracted with dichloromethane (2×50 mL). The combined organic layers were washed with saturated aqueous sodium chloride solution (30 mL), dried over sodium sulfate, filtered, and concentrated in vacuo; purification using silica gel chromatography (Gradient: 13% to 17% methanol in dichloromethane) provided P8 as a white solid. Yield: 765 mg, 1.71 mmol, 72%. LCMS m/z 449.2 [M+H]⁺. ¹H NMR (400 MHz, chloroform-d) δ 7.57 (ddd, J=11.8, 6.8, 2.3 Hz, 1H), 7.33 (d, J=8.6 Hz, 2H), 6.87 (d, J=8.5 Hz, 2H), 6.55-6.44 (m, 1H), 5.24 (s, 2H), 3.80 (s, 3H), 3.23 (d, J=6.1 Hz, 2H), 1.71-1.60 (m, 6H), 1.59-1.49 (m, 6H).

### Preparation P9

### 4-({2,3,5-Trifluoro-4-[(4-methoxyphenyl)methoxy]benzamido}methyl)bicyclo[2.2.2]octane-1-carboxylic acid (P9)

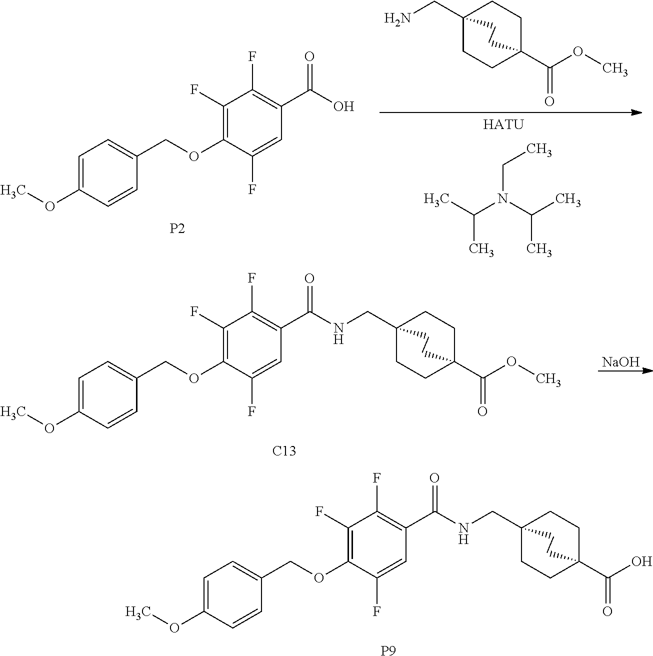

Step 1. Synthesis of methyl 4-({2,3,5-trifluoro-4-[(4-methoxyphenyl)methoxy]benzamido}methyl)bicyclo[2.2.2]octane-1-carboxylate (C13): To a solution of P2 (8.00 g, 25.6 mmol) and methyl 4-(aminomethyl)bicyclo[2.2.2]octane-1-carboxylate (5.05 g, 25.6 mmol) in N,N-dimethylformamide (60 mL) were added N,N-diisopropylethylamine (4.97 g, 38.4 mmol) and O-(7-azabenzotriazol-1-yl)-N,N,N,N′-tetramethyluronium hexafluorophosphate (HATU; 11.7 g, 30.8 mmol). After the reaction mixture had been stirred at room temperature for 4 hours, LCMS analysis indicated conversion to C13: LCMS m/z 492.2 [M+H]⁺. The reaction mixture was poured into ice water, and the solid was collected via filtration and washed with water, providing C13 as a gray solid. Yield: 11.6 g, 23.6 mmol, 92%.

Step 2. Synthesis of 4-({2,3,5-trifluoro-4-[(4-methoxyphenyl)methoxy]benzamido}methyl) bicyclo[2.2.2]octane-1-carboxylic acid (P9): A solution of C13 (11.6 g, 23.6 mmol) in methanol (120 mL) was treated with aqueous sodium hydroxide solution (3 M; 120 mL). The reaction mixture was stirred at 50° C. for 6 hours, then acidified by addition of hydrochloric acid. The resulting solid was collected via filtration and washed with water, then suspended in a mixture of ethyl acetate and methanol (10:1 ratio, 80 mL). This was stirred at 80° C., and treated slowly with methanol until a solution was obtained, whereupon it was cooled to room temperature. The resulting precipitate was collected via filtration and washed with ethyl acetate to afford P9 as a white solid. Yield: 9.0 g, 18.8 mmol, 80%. LCMS m/z 478.1 [M+H]⁺. ¹H NMR (400 MHz, DMSO-d₆) δ 8.26 (br t, J=6.2 Hz, 1H), 7.42-7.23 (m, 3H), 6.94 (d, J=8.3 Hz, 2H), 5.21 (s, 2H), 3.75 (s, 3H), 2.98 (d, J=6.2 Hz, 2H), 1.71-1.55 (m, 6H), 1.44-1.29 (m, 6H).

### Preparation P10

### 2,3,5-Trifluoro-N-{[4-(N-hydroxycarbamimidoyl)bicyclo[2.2.2]octan-1-yl]methyl}-4-[(4-methoxyphenyl)methoxy]benzamide (P10)

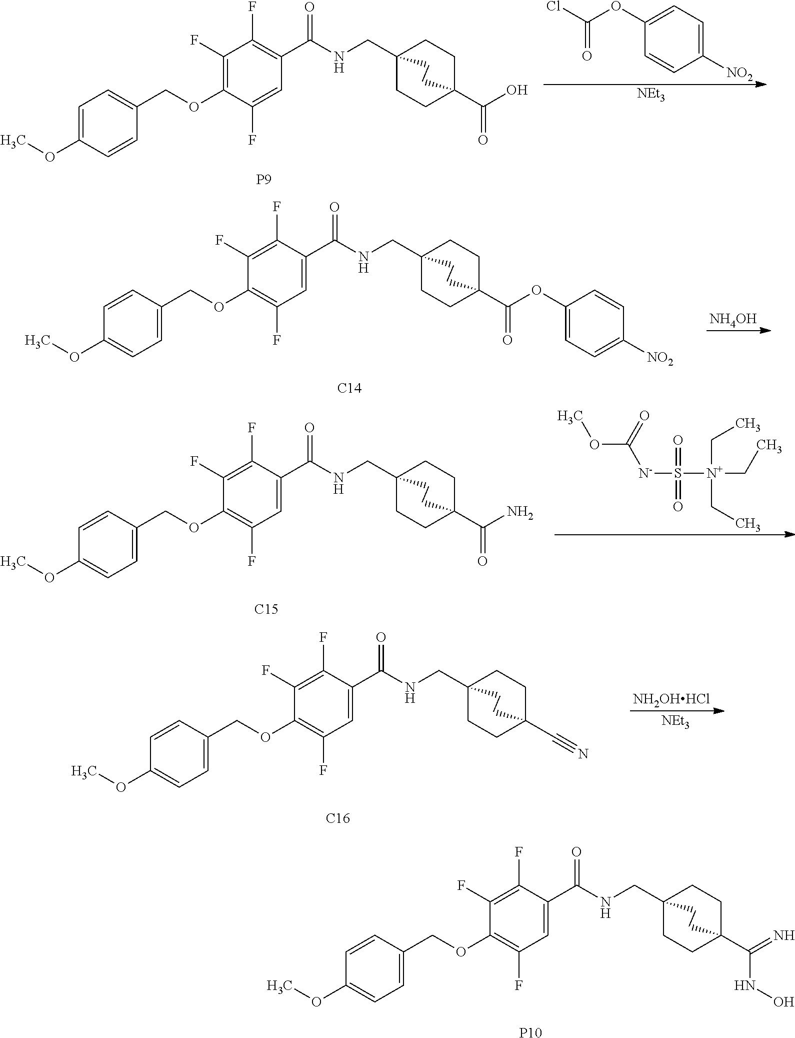

Step 1. Synthesis of 4-nitrophenyl 4-({2,3,5-trifluoro-4-[(4-methoxyphenyl)methoxy]benzamido}methyl) bicyclo[2.2.2]octane-1-carboxylate (C14): To a 0° C. suspension of P9 (962 mg, 2.01 mmol) in dichloromethane (8 mL) was added 4-nitrophenyl chloroformate (425 mg, 2.11 mmol), followed by triethylamine (0.842 mL, 6.04 mmol). The reaction mixture was allowed to warm to room temperature, then stir overnight at room temperature, whereupon it was concentrated in vacuo, providing C14 as a solid (1.20 g). This material was taken directly to the following step. LCMS m/z 599.4 [M+H]⁺. ¹H NMR (400 MHz, DMSO-d₆), characteristic peaks: δ 5.21 (s, 2H), 3.75 (s, 3H), 3.05 (d, J=6.3 Hz, 2H), 1.93-1.83 (m, 6H), 1.53-1.43 (m, 6H).

Step 2. Synthesis of 4-({2,3,5-trifluoro-4-[(4-methoxyphenyl)methoxy]benzamido}methyl)bicyclo[2.2.2]octane-1-carboxamide (C15): A solution of C14 (from the previous step; 1.20 g, 52.01 mmol) in N,N-dimethylformamide (10 mL) was treated with concentrated ammonium hydroxide (14.5 M; 0.415 mL, 6.02 mmol), and the reaction mixture was stirred at room temperature for 5 hours. It was then added to water (100 mL) and the resulting mixture was extracted with ethyl acetate (3×80 mL); the combined organic layers were washed with saturated aqueous sodium chloride solution, dried over sodium sulfate, filtered, and concentrated in vacuo to provide C15 as an off-white solid. Yield: 919 mg, 1.93 mmol, 96% over 2 steps. LCMS m/z 477.3 [M+H]⁺. ¹H NMR (400 MHz, DMSO-d₆) δ 8.27 (br t, J=6 Hz, 1H), 7.35 (d, J=8.6 Hz, 2H), 7.30 (ddd, J=11.0, 6.1, 2.4 Hz, 1H), 6.94 (d, J=8.6 Hz, 2H), 6.88 (br s, 1H), 6.66 (br s, 1H), 5.21 (s, 2H), 3.75 (s, 3H), 2.99 (d, J=6.2 Hz, 2H), 1.66-1.57 (m, 6H), 1.41-1.33 (m, 6H).

Step 3. Synthesis of N-[(4-cyanobicyclo[2.2.2]octan-1-yl)methyl]-2,3,5-trifluoro-4-[(4-methoxyphenyl)methoxy]benzamide (C16): To a solution of C15 (797 mg, 1.67 mmol) in ethyl acetate (10 mL) was added (methoxycarbonylsulfamoyl)triethylammonium hydroxide, inner salt (Burgess reagent; 997 mg, 4.18 mmol). The reaction mixture was stirred at room temperature overnight, whereupon it was diluted with ethyl acetate (40 mL) and washed sequentially with water (2×30 mL) and saturated aqueous sodium chloride solution (30 mL). The organic layer was then dried over sodium sulfate, filtered, and concentrated in vacuo, affording C16 as a solid. Yield: 658 mg, 1.44 mmol, 86%. LCMS m/z 459.3 [M+H]⁺. ¹H NMR (400 MHz, DMSO-d₆) δ 8.32 (br t, J=6.3 Hz, 1H), 7.35 (d, J=8.7 Hz, 2H), 7.34-7.28 (m, 1H), 6.94 (d, J=8.6 Hz, 2H), 5.21 (s, 2H), 3.75 (s, 3H), 2.99 (d, J=6.3 Hz, 2H), 1.88-1.79 (m, 6H), 1.46-1.37 (m, 6H).

Step 4. Synthesis of 2,3,5-trifluoro-N-{[4-(N-hydroxycarbamimidoyl)bicyclo[2.2.2]octan-1-yl]methyl}-4-[(4-methoxyphenyl)methoxy]benzamide (P10): To a suspension of C16 (658 mg, 1.44 mmol) in methanol (8.0 mL) was added triethylamine (0.440 mL, 3.16 mmol), followed by hydroxylamine hydrochloride (219 mg, 3.15 mmol). No reaction was observed over several hours at room temperature. Hydroxylamine hydrochloride (219 mg, 3.15 mmol) was again added and the reaction mixture was heated at 50° C. for 24 hours. After cooling, it was diluted with ethyl acetate (30 mL) and washed sequentially with water (2×40 mL) and saturated aqueous sodium chloride solution (30 mL). The organic layer was then dried over sodium sulfate, filtered, and concentrated in vacuo, providing P10 as a solid. Yield: 330 mg, 0.671 mmol, 47%. LCMS m/z 492.4 [M+H]⁺. ¹H NMR (400 MHz, DMSO-d₆) δ 11.82 (br s, 1H), 10.64 (br s, 1H), 8.59 (v br s, 1H), 8.33 (br t, J=6.3 Hz, 1H), 7.35 (d, J=8.6 Hz, 2H), 7.34-7.28 (m, 1H), 6.94 (d, J=8.6 Hz, 2H), 5.21 (s, 2H), 3.75 (s, 3H), 3.03 (d, J=6.3 Hz, 2H), 1.78-1.67 (m, 6H), 1.49-1.38 (m, 6H).

### Preparation P11

### tert-Butyl {[(1r,4r)-4-(7-bromoimidazo[1,2-a]pyridin-2-yl)cyclohexyl]methyl}carbamate (P11)

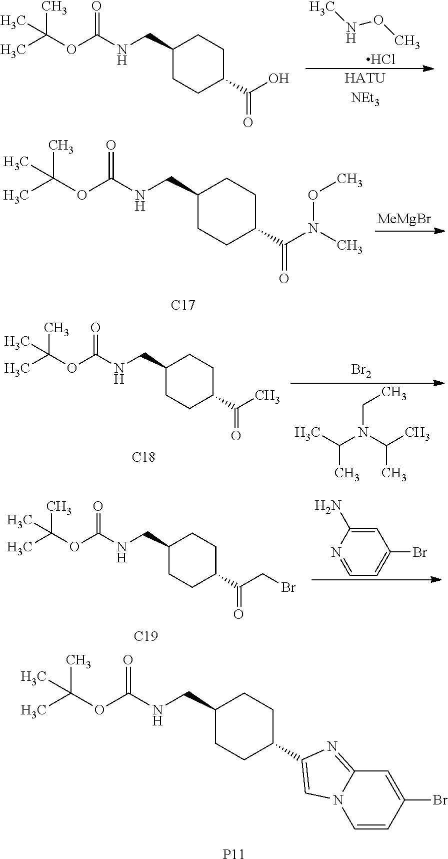

Step 1. Synthesis of tert-butyl ({(1r,4r)-4-[methoxy(methyl)carbamoyl]cyclohexyl}methyl)carbamate (C17): To a solution of (1r,4r)-4-{[(tert-butoxycarbonyl)amino]methyl}cyclohexane-1-carboxylic acid (10.2 g, 39.6 mmol) in N,N-dimethylformamide (100 mL) were added N,O-dimethylhydroxylamine hydrochloride (4.66 g, 47.8 mmol), O-(7-azabenzotriazol-1-yl)-N,N,N′,N′-tetramethyluronium hexafluorophosphate (HATU; 19.7 g, 51.8 mmol), and triethylamine (16.7 mL, 120 mmol). After the reaction mixture had been stirred overnight at room temperature, LCMS analysis indicated formation of C17: LCMS m/z 301.5 [M+H]⁺. In pilot reactions run on smaller scale, the reaction mixture was then concentrated under reduced pressure, diluted with a 1:1 mixture of ethyl acetate and dichloromethane, and filtered; concentration of the filtrate in vacuo provided C17. The product from this 39.6 mmol-scale reaction was combined with that from a similar reaction carried out using (1r,4r)-4-{[(tert-butoxycarbonyl)amino]methyl}cyclohexane-1-carboxylic acid (9.50 g, 36.9 mmol) to provide C17 as an oil. Combined yield: 22.8 g, 75.9 mmol, 99%. ¹H NMR (500 MHz, chloroform-d) δ 4.56 (br s, 1H), 3.68 (s, 3H), 3.16 (s, 3H), 2.98 (br d, J=6.4 Hz, 2H), 2.69-2.57 (m, 1H), 1.87-1.77 (m, 4H), 1.57-1.38 (m, 3H), 1.44 (s, 9H), 1.05-0.94 (m, 2H).

Step 2. Synthesis of tert-butyl {[(1r,4r)-4-acetylcyclohexyl]methyl}carbamate (C18): Methylmagnesium bromide (3.0 M; 81.7 mL, 245 mmol) was added drop-wise to a 0° C. solution of C17 (23.0 g, 76.6 mmol) in tetrahydrofuran (219 mL), whereupon the reaction mixture was allowed to warm to room temperature. After 2 hours, it was cooled to 0° C., treated with water (50 mL), and then diluted with ethyl acetate. The aqueous layer was extracted twice with ethyl acetate, and the combined organic layers were washed with saturated aqueous sodium chloride solution, dried over sodium sulfate, filtered, and concentrated in vacuo. Silica gel chromatography (Eluents: 0%, then 10%, then 25%, then 50% ethyl acetate in heptane) afforded C18 as a solid. Yield: 13.3 g, 52.1 mmol, 68%. ¹H NMR (500 MHz, chloroform-d) δ 4.56 (br s, 1H), 2.98 (br dd, J=6, 6 Hz, 2H), 2.28 (tt, J=12.2, 3.5 Hz, 1H), 2.13 (s, 3H), 1.98-1.91 (m, 2H), 1.88-1.81 (m, 2H), 1.44 (s, 9H), 1.44-1.37 (m, 1H), 1.37-1.26 (m, 2H), 1.02-0.92 (m, 2H).

Step 3. Synthesis of tert-butyl {[(1r,4r)-4-(bromoacetyl)cyclohexyl]methyl}carbamate (C19): Bromine (2.57 mL, 50.2 mmol) was added portion-wise to a 0° C. solution of C18 (12.1 g, 47.4 mmol) in methanol (158 mL). After the mixture had been stirred at 0° C. for 1 hour, and at room temperature for 1 hour, N,N-diisopropylethylamine (29.6 mL, 170 mmol) was added in a portion-wise manner. Stirring was continued at room temperature for 20 minutes, whereupon the mixture was concentrated in vacuo and combined with the product of a similar reaction carried out using C18 (1.03 g, 4.03 mmol). Silica gel chromatography (Eluents: 0%, then 10%, then 25% ethyl acetate in heptane) provided C19 as a solid. Combined yield: 9.07 g, 27.1 mmol, 53%. ¹H NMR (500 MHz, chloroform-d) δ 4.56 (br s, 1H), 3.95 (s, 2H), 2.99 (dd, J=6, 6 Hz, 2H), 2.68 (tt, J=12.1, 3.4 Hz, 1H), 2.00-1.92 (m, 2H), 1.90-1.82 (m, 2H), 1.48-1.34 (m, 3H), 1.44 (s, 9H), 1.06-0.95 (m, 2H).

Step 4. Synthesis of tert-butyl {[(1r,4r)-4-(7-bromoimidazo[1,2-a]pyridin-2-yl)cyclohexyl]methyl}carbamate (P11): A suspension of C19 (1.00 g, 2.99 mmol) and 4-bromopyridin-2-amine (1.04 g, 6.01 mmol) in ethanol (20 mL) was heated at 70° C. overnight. After the reaction mixture had cooled to room temperature, it was poured into water (150 mL) with stirring, and stirred for 20 minutes. Solids were collected via filtration and washed with water to afford P11 as a white solid. Yield: 976 mg, 2.39 mmol, 80%. LCMS m/z 408.2 (bromine isotope pattern observed) [M+H]⁺. ¹H NMR (400 MHz, DMSO-d₆) δ 8.41 (br d, J=7.1 Hz, 1H), 7.75 (br d, J=2.0 Hz, 1H), 7.69 (s, 1H), 6.97 (dd, J=7.2, 2.0 Hz, 1H), 6.83 (br t, J=5.9 Hz, 1H), 2.81 (dd, J=6, 6 Hz, 2H), 2.64-2.53 (m, 1H), 2.09-1.99 (m, 2H), 1.82-1.73 (m, 2H), 1.45-1.29 (m, 3H), 1.38 (s, 9H), 1.08-0.94 (m, 2H).

### Preparation P12

### tert-Butyl ({(1r,4r)-4-[6-(4,4,5,5-tetramethyl-1,3,2-dioxaborolan-2-yl)-2H-indazol-2-yl]cyclohexyl}methyl)carbamate (P12)

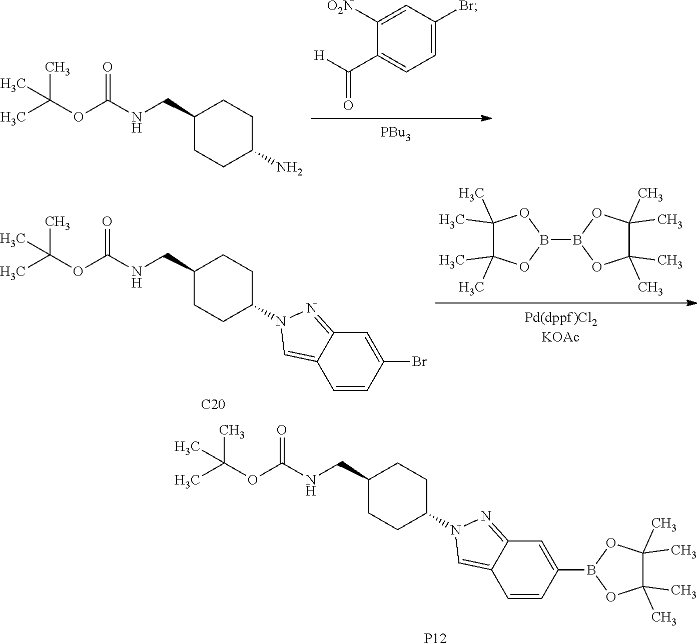

Step 1. Synthesis of tert-butyl {[(1r,4r)-4-(6-bromo-2H-indazol-2-yl)cyclohexyl]methyl}carbamate (C20): A suspension of tert-butyl {[(1r,4r)-4-aminocyclohexyl]methyl}carbamate (5.00 g, 21.9 mmol) and 4-bromo-2-nitrobenzaldehyde (5.04 g, 21.9 mmol) in propan-2-ol (70 mL) was heated at 80° C. for 4 hours. After the reaction mixture had cooled to room temperature, tributylphosphine (94%, 12 mL, 45 mmol) was added via syringe over 5 minutes; the reaction mixture was then heated at 80° C. overnight. Upon cooling to room temperature, the reaction mixture was filtered, and the filter cake was washed with heptane to afford C20 as a tan solid. Yield: 6.52 g, 16.0 mmol, 73%. LCMS m/z 408.1 (bromine isotope pattern observed) [M+H]⁺. ¹H NMR (400 MHz, DMSO-d₆) 8.45 (s, 1H), 7.86-7.82 (m, 1H), 7.67 (d, J=8.8 Hz, 1H), 7.12 (dd, J=8.8, 1.7 Hz, 1H), 6.89 (br t, J=6.0 Hz, 1H), 4.50-4.38 (m, 1H), 2.85 (dd, J=6.3, 6.3 Hz, 2H), 2.17-2.07 (m, 2H), 1.93-1.78 (m, 4H), 1.54-1.41 (m, 1H), 1.39 (s, 9H), 1.20-1.04 (m, 2H).

Step 2. Synthesis of tert-butyl ({(1r,4r)-4-[6-(4,4,5,5-tetramethyl-1,3,2-dioxaborolan-2-yl)-2H-indazol-2-yl]cyclohexyl}methyl)carbamate (P12): A mixture of C20 (6.52 g, 16.0 mmol), 4,4,4′,4′,5,5,5′,5′-octamethyl-2,2′-bi-1,3,2-dioxaborolane (6.08 g, 23.9 mmol), and potassium acetate (95%, 4.95 g, 47.9 mmol) in 1,4-dioxane (200 mL) was degassed with nitrogen for 10 minutes, whereupon [1,1′-bis(diphenylphosphino)ferrocene]dichloropalladium(II), dichloromethane complex (Pd(dppf)Cl₂; 652 mg, 0.798 mmol) was added. After the reaction mixture had been heated at 100° C. for 1 hour, it was allowed to cool and filtered through a pad of diatomaceous earth. The filter cake was rinsed with ethyl acetate, and the combined filtrates were concentrated in vacuo; silica gel chromatography (Gradient: 30% to 70% ethyl acetate in heptane; loaded as a solution in dichloromethane) provided P12 as a colorless foam. Yield: 7.20 g, 15.8 mmol, 99%. LCMS m/z 456.4 [M+H]⁺. ¹H NMR (400 MHz, DMSO-d₆), characteristic peaks: δ 8.40 (s, 1H), 7.96-7.93 (m, 1H), 7.65 (br d, J=8.4 Hz, 1H), 7.25 (br d, J=8.4 Hz, 1H), 6.89 (br t, J=6.0 Hz, 1H), 4.53-4.39 (m, 1H), 2.85 (dd, J=6, 6 Hz, 2H), 2.19-2.08 (m, 2H), 1.93-1.78 (m, 4H), 1.56-1.43 (m, 1H), 1.39 (s, 9H), 1.31 (s, 12H).

### Preparation P13

### 1-{(1r,4r)-4-[6-(1-Methyl-1H-pyrazol-4-yl)-2H-indazol-2-yl]cyclohexyl}methanamine, hydrochloride salt (P13)

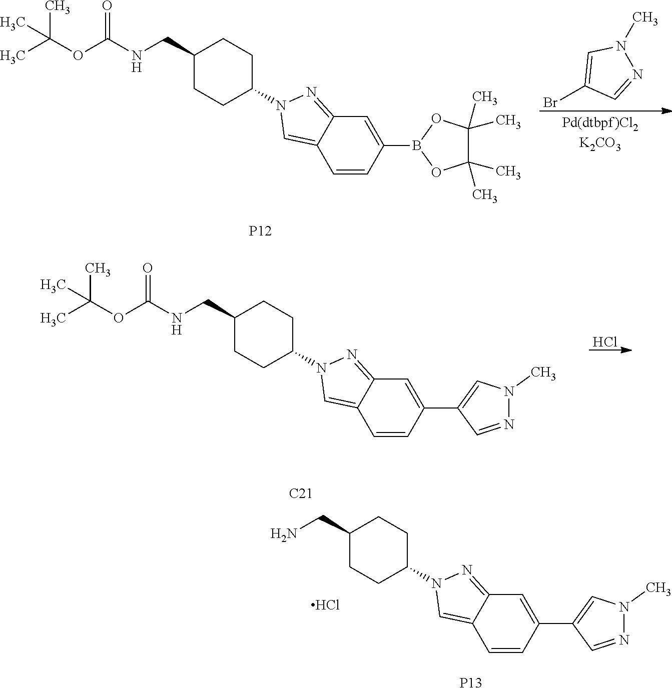

Step 1. Synthesis of tert-butyl ({(1r,4r)-4-[6-(1-methyl-1H-pyrazol-4-yl)-2H-indazol-2-yl]cyclohexyl}methyl)carbamate (C21): 4-Bromo-1-methyl-1H-pyrazole (233 mg, 1.45 mmol), P12 (600 mg, 1.32 mmol), aqueous potassium carbonate solution (2 M; 1.98 mL, 3.96 mmol), [1,1′-bis(di-tert-butylphosphino)ferrocene]dichloropalladium(II) [Pd(dtbpf)Cl₂; 85.9 mg, 0.132 mmol], ethanol (5 mL), and water (1 mL) were combined in a pressure-relief vial, and the reaction mixture was heated at 85° C. for 1 hour. After the reaction mixture had cooled, concentration in vacuo was used to remove ethanol, and the resulting mixture was partitioned between ethyl acetate and water. The organic layer was washed with saturated aqueous sodium chloride solution, dried over magnesium sulfate, filtered, and concentrated in vacuo; silica gel chromatography (Eluents: ethyl acetate, then 5% methanol in ethyl acetate) afforded C21 as a colorless foam. Yield: 404 mg, 0.986 mmol, 75%. LCMS m/z 410.4 [M+H]⁺. ¹H NMR (400 MHz, DMSO-d₆) δ 8.31 (br s, 1H), 8.15 (s, 1H), 7.91-7.89 (m, 1H), 7.76-7.72 (m, 1H), 7.65 (dd, J=8.6, 0.9 Hz, 1H), 7.25 (dd, J=8.7, 1.4 Hz, 1H), 6.90 (br t, J=5.9 Hz, 1H), 4.47-4.34 (m, 1H), 3.87 (s, 3H), 2.85 (dd, J=6, 6 Hz, 2H), 2.18-2.07 (m, 2H), 1.94-1.78 (m, 4H), 1.54-1.42 (m, 1H), 1.39 (s, 9H), 1.20-1.05 (m, 2H).

Step 2. Synthesis of 1-{(1r,4r)-4-[6-(1-methyl-1H-pyrazol-4-yl)-2H-indazol-2-yl]cyclohexyl}methanamine, hydrochloride salt (P13): A solution of hydrogen chloride in 1,4-dioxane (4 M; 6 mL) was added to C21 (404 mg, 0.986 mmol). Propan-2-ol (3 mL) was added to aid solubility and stirring, and the reaction mixture was allowed to stir overnight, whereupon it was diluted with diethyl ether (50 mL). Solids were collected via filtration and washed with diethyl ether to provide P13 as a solid. Yield: 362 mg, assumed quantitative. LCMS m/z 310.3 [M+H]⁺. ¹H NMR (400 MHz, DMSO-d₆) δ 8.37 (br s, 1H), 8.17 (s, 1H), 7.92 (s, 1H), 7.74 (br s, 1H), 7.66 (br d, J=8.6 Hz, 1H), 7.27 (dd, J=8.7, 1.4 Hz, 1H), 4.50-4.39 (m, 1H), 3.87 (s, 3H), 2.78-2.68 (m, 2H), 2.22-2.12 (m, 2H), 2.01-1.84 (m, 4H), 1.78-1.65 (m, 1H), 1.29-1.15 (m, 2H).

### Preparation P14

### 3,5-Difluoro-4-[(4-methoxyphenyl)methoxy]-N-({(1r,4r)-4-[6-(4,4,5,5-tetramethyl-1,3,2-dioxaborolan-2-yl)-2H-indazol-2-yl]cyclohexyl}methyl)benzamide (P14)

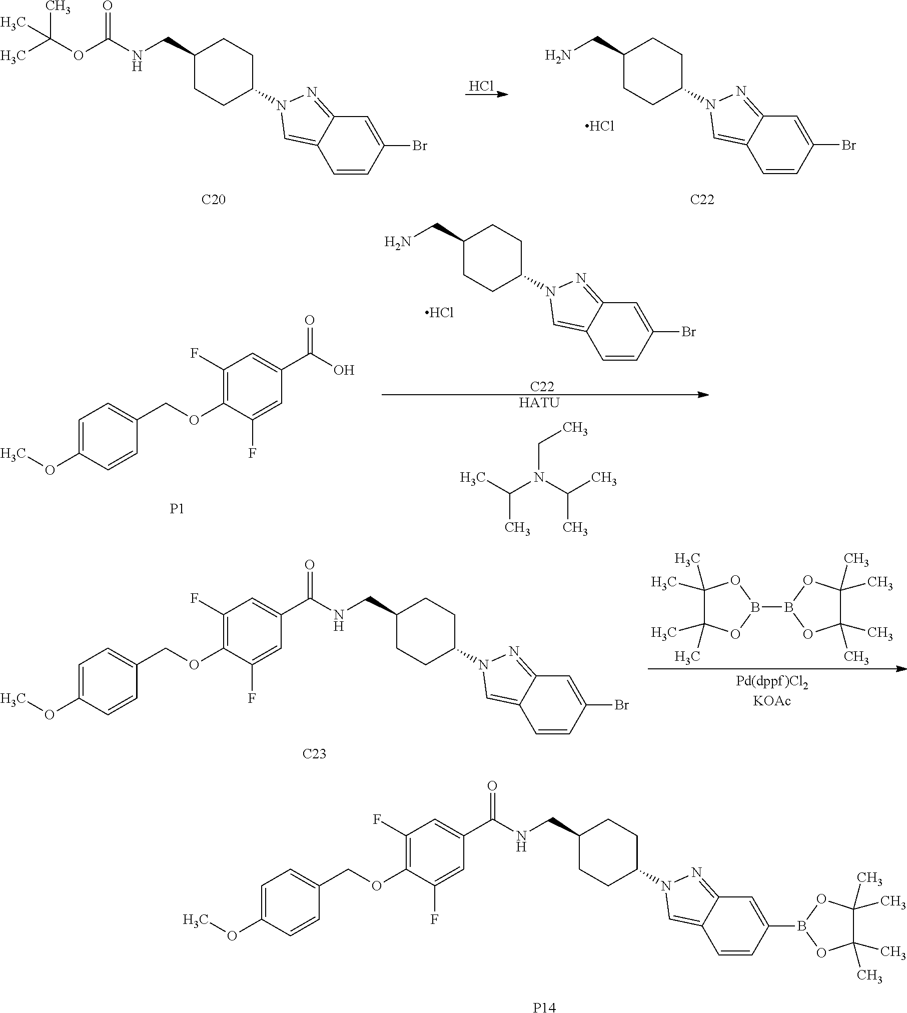

Step 1. Synthesis of 1-[(1r,4r)-4-(6-bromo-2H-indazol-2-yl)cyclohexyl]methanamine, hydrochloride salt (C22): A solution of hydrogen chloride in 1,4-dioxane (4 M; 25 mL, 100 mmol) was added to a solution of C20 (7.35 g, 18.0 mmol) in 1,4-dioxane (30 mL); the reaction mixture was stirred at room temperature for 3 hours, followed by overnight at 50° C. After the reaction mixture had cooled, it was diluted with diethyl ether (100 mL). Solids were collected via filtration and washed with diethyl ether, affording C22 as a solid. Yield: 6.20 g, 18.0 mmol, quantitative. LCMS m/z 308.5 (bromine isotope pattern observed) [M+H]⁺. ¹H NMR (400 MHz, DMSO-d₆) δ 8.49 (br s, 1H), 8.06 (br s, 3H), 7.86-7.83 (m, 1H), 7.68 (d, J=8.8 Hz, 1H), 7.12 (dd, J=8.8, 1.7 Hz, 1H), 4.47 (tt, J=11.7, 3.9 Hz, 1H), 2.78-2.66 (m, 2H), 2.21-2.11 (m, 2H), 2.01-1.82 (m, 4H), 1.78-1.65 (m, 1H), 1.29-1.14 (m, 2H).

Step 2. Synthesis of N-{[(1r,4r)-4-(6-bromo-2H-indazol-2-yl)cyclohexyl]methyl}-3,5-difluoro-4-[(4-methoxyphenyl)methoxy]benzamide (C23): To a suspension of C22 (6.20 g, 18.0 mmol) and P1 (5.92 g, 20.1 mmol) in N,N-dimethylformamide (15 mL) was added N,N-diisopropylethylamine (14 mL, 80.4 mmol), followed by O-(7-azabenzotriazol-1-yl)-N,N,N,N′-tetramethyluronium hexafluorophosphate (HATU, 95%; 9.66 g, 24.1 mmol). The reaction mixture was stirred at room temperature for 3 days, whereupon it was poured into water (450 mL) with stirring. The resulting solid was isolated via filtration and washed with water, providing C23 as a solid. Yield: 10.0 g, 17.1 mmol, 95%. LCMS m/z 584.2 (bromine isotope pattern observed) [M+H]⁺. ¹H NMR (400 MHz, DMSO-d₆) δ 8.57 (br t, J=5.8 Hz, 1H), 8.45 (br s, 1H), 7.86-7.83 (m, 1H), 7.67 (d, J=8.8 Hz, 1H), 7.65-7.56 (m, 2H), 7.34 (d, J=8.6 Hz, 2H), 7.12 (dd, J=8.8, 1.7 Hz, 1H), 6.92 (d, J=8.6 Hz, 2H), 5.17 (s, 2H), 4.53-4.41 (m, 1H), 3.74 (s, 3H), 3.17 (dd, J=6, 6 Hz, 2H), 2.21-2.09 (m, 2H), 1.96-1.80 (m, 4H), 1.73-1.59 (m, 1H), 1.28-1.13 (m, 2H).

Step 3. Synthesis of 3,5-difluoro-4-[(4-methoxyphenyl)methoxy]-N-({(1r,4r)-4-[6-(4,4,5,5-tetramethyl-1,3,2-dioxaborolan-2-yl)-2H-indazol-2-yl]cyclohexyl}methyl)benzamide (P14): A mixture of C23 (10 g, 17.1 mmol), 4,4,4′,4′,5,5,5′,5′-octamethyl-2,2′-bi-1,3,2-dioxaborolane (6.52 g, 25.7 mmol), and potassium acetate (95%, 5.30 g, 51.3 mmol), in 1,4-dioxane (250 mL) was degassed with nitrogen for 10 minutes. [1,1′-Bis(diphenylphosphino)ferrocene]dichloropalladium(II), dichloromethane complex (700 mg, 0.857 mmol) was added and the reaction mixture was purged with nitrogen for an additional 5 minutes, whereupon it was heated at 100° C. for 2 hours. After the reaction mixture had cooled, it was filtered through diatomaceous earth and the filter pad was rinsed with ethyl acetate. The combined filtrates were concentrated in vacuo, and the residue was purified via silica gel chromatography (Gradient: 2% to 10% methanol in dichloromethane; loaded as a solution in dichloromethane) to afford P14 as a tan solid. Yield: 7.32 g, 11.6 mmol, 68%. LCMS m/z 632.3 [M+H]⁺. ¹H NMR (400 MHz, DMSO-d₆) δ 8.57 (brt, J=5.8 Hz, 1H), 8.40 (br s, 1H), 7.96-7.94 (m, 1H), 7.65 (dd, J=8.4, 1 Hz, 1H), 7.66-7.56 (m, 2H), 7.34 (d, J=8.6 Hz, 2H), 7.25 (br d, J=8.4 Hz, 1H), 6.92 (d, J=8.7 Hz, 2H), 5.17 (s, 2H), 4.55-4.43 (m, 1H), 3.74 (s, 3H), 3.18 (dd, J=6, 6 Hz, 2H), 2.21-2.10 (m, 2H), 1.96-1.81 (m, 4H), 1.75-1.60 (m, 1H), 1.31 (s, 12H), 1.28-1.14 (m, 2H).

### Preparation P15

### N-{[(1r,4r)-4-(6-Chloro-2H-pyrazolo[4,3-c]pyridin-2-yl)cyclohexyl]methyl}-3,5-difluoro-4-[(4-methoxyphenyl)methoxy]benzamide (P15)

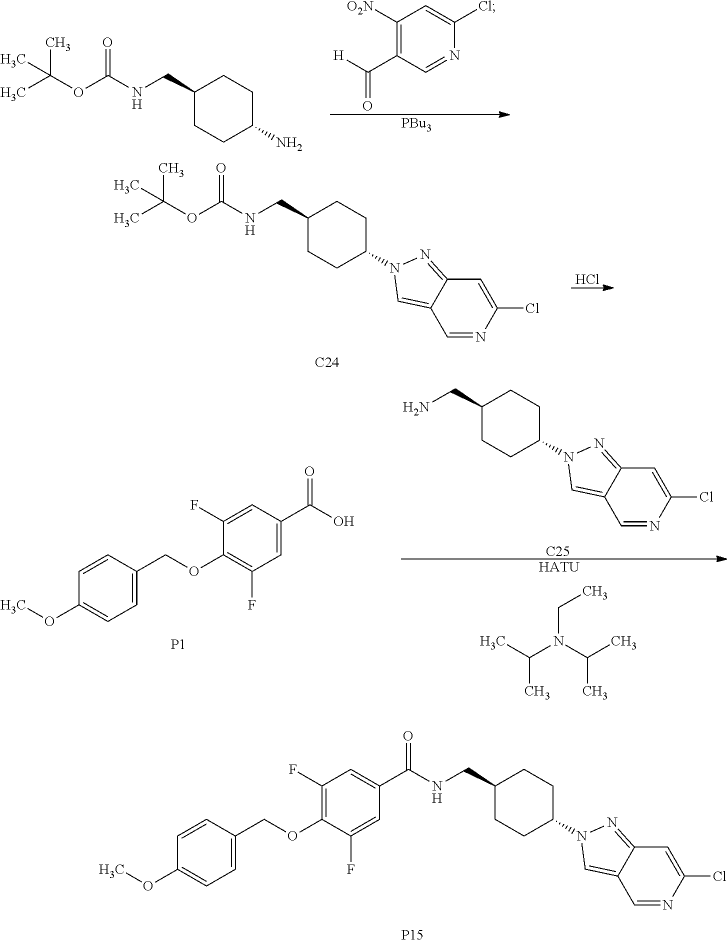

Step 1. Synthesis of tert-butyl {[(1r,4r)-4-(6-chloro-2H-pyrazolo[4,3-c]pyridin-2-yl)cyclohexyl]methyl}carbamate (C24): A mixture of 6-chloro-4-nitropyridine-3-carbaldehyde (2.00 g, 10.7 mmol) and tert-butyl {[(1r,4r)-4-aminocyclohexyl]methyl}carbamate (2.45 g, 10.7 mmol) in propan-2-ol (50 mL) was heated at 80° C. for 4 hours, whereupon the reaction mixture was cooled to room temperature. Tributylphosphine (6.51 g, 32.2 mmol) was then added, and the reaction mixture was heated at 80° C. for an additional 6 hours. After removal of solvent in vacuo, the residue was purified via reversed-phase HPLC (Column: Waters XBridge C18, 30×150 mm, 5 μm; Mobile phase A: water containing 0.05% formic acid; Mobile phase B: acetonitrile; Gradient: 50% to 60% B; Flow rate: 20 mL/minute) to provide C24 as a white solid. Yield: 260 mg, 0.713 mmol, 7%. LCMS m/z 365.2 (chlorine isotope pattern observed) [M+H]⁺. ¹H NMR (400 MHz, DMSO-d₆) δ 9.01 (d, J=1.2 Hz, 1H), 8.81 (br s, 1H), 7.68 (br s, 1H), 6.91 (br t, J=5.9 Hz, 1H), 4.59-4.47 (m, 1H), 2.85 (dd, J=6, 6 Hz, 2H), 2.20-2.08 (m, 2H), 1.95-1.78 (m, 4H), 1.54-1.40 (m, 1H), 1.38 (s, 9H), 1.20-1.04 (m, 2H).

Step 2. Synthesis of 1-[(1r,4r)-4-(6-chloro-2H-pyrazolo[4,3-c]pyridin-2-yl)cyclohexyl]methanamine, hydrochloride salt (C25): To a solution of C24 (260 mg, 0.713 mmol) in dichloromethane (4 mL) was added a solution of hydrogen chloride in 1,4-dioxane (4 M; 1 mL, 4 mmol). After the reaction mixture had been stirred at 15° C. for 4 hours, it was concentrated in vacuo to afford C25 as a yellow oil. Yield: 170 mg, 0.564 mmol, 79%. LCMS m/z 265.1 (chlorine isotope pattern observed) [M+H]⁺.

Step 3. Synthesis of N-{[(1r,4r)-4-(6-chloro-2H-pyrazolo[4,3-c]pyridin-2-yl)cyclohexyl]methyl}-3,5-difluoro-4-[(4-methoxyphenyl)methoxy]benzamide (P15): To a solution of P1 (186 mg, 0.632 mmol), N,N-diisopropylethylamine (163 mg, 1.26 mmol), and O-(7-azabenzotriazol-1-yl)-N,N,N′,N′-tetramethyluronium hexafluorophosphate (HATU; 288 mg, 0.757 mmol) in dichloromethane (10 mL) was added C25 (167 mg, 0.554 mmol), whereupon the reaction mixture was stirred at 15° C. for 1 hour. It was then extracted with dichloromethane (3×30 mL), and the combined organic layers were washed with saturated aqueous sodium chloride solution, dried over sodium sulfate, filtered, and concentrated in vacuo. Purification using silica gel chromatography (Eluent: 4% methanol in dichloromethane) provided P15 as a yellow oil. Yield: 250 mg, 0.462 mmol, 83%. LCMS m/z 541.1 (chlorine isotope pattern observed) [M+H]⁺. ¹H NMR (400 MHz, DMSO-d₆), characteristic peaks: δ 9.01 (d, J=1.2 Hz, 1H), 8.80 (br s, 1H), 8.58 (br t, J=5.8 Hz, 1H), 7.68 (br s, 1H), 7.66-7.56 (m, 2H), 7.34 (d, J=8.6 Hz, 2H), 6.92 (d, J=8.7 Hz, 2H), 5.17 (s, 2H), 4.63-4.49 (m, 1H), 3.74 (s, 3H), 2.21-2.10 (m, 2H).

### Preparation P16

### N-{[4-(6-Bromo-2H-indazol-2-yl)bicyclo[2.2.2]octan-1-yl]methyl}-2,3,5-trifluoro-4-[(4-methoxyphenyl)methoxy]benzamide (P16)

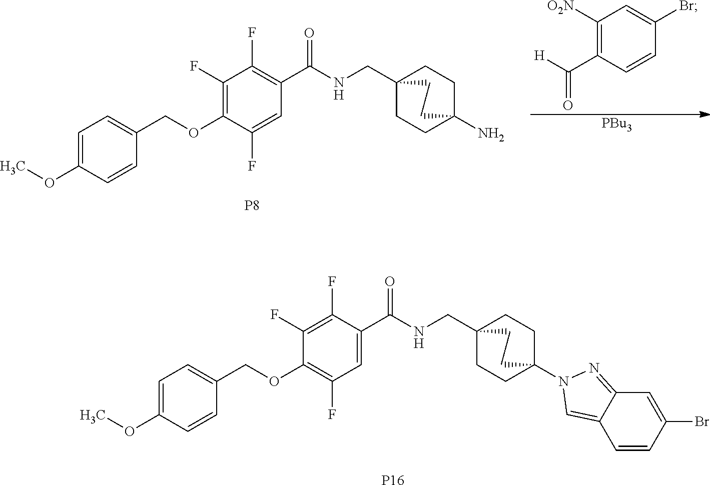

A solution of P8 (200 mg, 0.446 mmol) and 4-bromo-2-nitrobenzaldehyde (123 mg, 0.535 mmol) in propan-2-ol (10 mL) was stirred at 85° C. for 4 hours, whereupon it was cooled to room temperature and treated with tributylphosphine (2 mL, 8 mmol). After the reaction mixture had been stirred at 85° C. overnight, it was diluted with water (15 mL) and extracted with dichloromethane (3×10 mL). The combined organic layers were washed with saturated aqueous sodium chloride solution (100 mL), dried over sodium sulfate, filtered, concentrated in vacuo, and subjected to chromatography on silica gel (Gradient: 0% to 10% methanol in dichloromethane), affording P16 as a yellow solid. ¹H NMR data was obtained from a reaction carried out in a similar manner. Yield: 170 mg, 0.270 mmol, 61%. LCMS m/z 626.1 (bromine isotope pattern observed) [M−H]⁻. ¹H NMR (400 MHz, DMSO-d₆) δ 8.46 (d, J=1.0 Hz, 1H), 8.40 (br t, J=6.3 Hz, 1H), 7.86-7.83 (m, 1H), 7.66 (br d, J=8.8 Hz, 1H), 7.38-7.32 (m, 1H), 7.36 (d, J=8.7 Hz, 2H), 7.11 (dd, J=8.8, 1.7 Hz, 1H), 6.94 (d, J=8.7 Hz, 2H), 5.22 (s, 2H), 3.75 (s, 3H), 3.10 (d, J=6.2 Hz, 2H), 2.20-2.10 (m, 6H), 1.71-1.60 (m, 6H).

### Preparation P17

### N-{[(1r,4r)-4-(1,3-Benzoxazol-2-yl)cyclohexyl]methyl}-3,5-difluoro-4-hydroxybenzamide (P17)

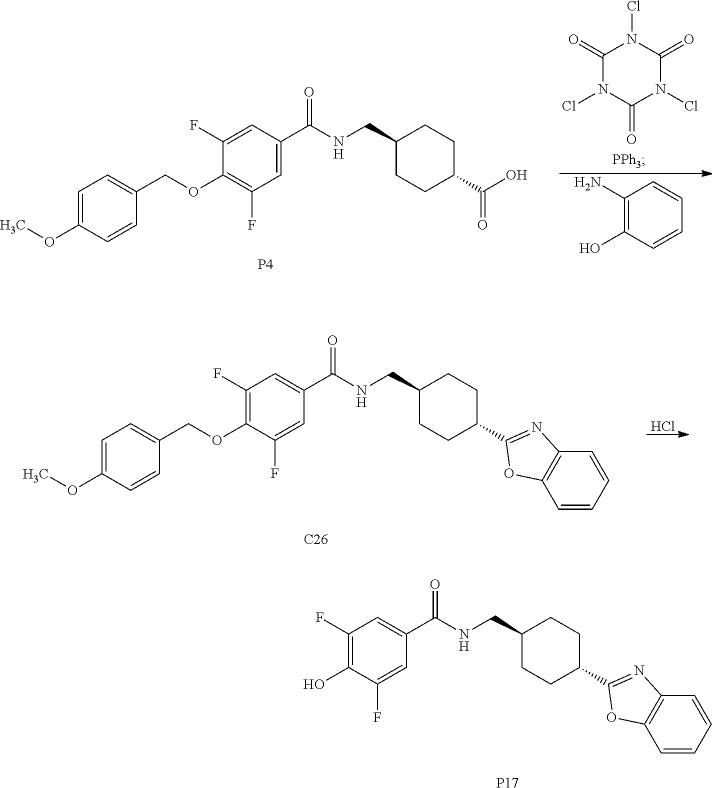

Step 1. Synthesis of N-{[(1r,4r)-4-(1,3-benzoxazol-2-yl)cyclohexyl]methyl}-3,5-difluoro-4-[(4-methoxyphenyl)methoxy]benzamide (C26): 1,3,5-Trichloro-1,3,5-triazinane-2,4,6-trione (161 mg, 0.693 mmol) and P4 (1.00 g, 2.31 mmol) were added to a 0° C. mixture of triphenylphosphine (95%, 637 mg, 2.31 mmol) in 1,4-dioxane (40 mL). After the reaction mixture had been stirred for 30 minutes, it was warmed to room temperature, 2-aminophenol (378 mg, 3.46 mmol) was added, and the reaction mixture was stirred overnight at 105° C. Once it had cooled, the reaction mixture was filtered through a pad of diatomaceous earth, and the pad was rinsed sequentially with 1,4-dioxane, ethyl acetate, and dichloromethane. The combined filtrates were concentrated in vacuo to afford C26 as an orange oil, which was progressed directly to the following step. LCMS m/z 507.3 [M+H]⁺.

Step 2. Synthesis of N-{[(1r,4r)-4-(1,3-benzoxazol-2-yl)cyclohexyl]methyl}-3,5-difluoro-4-hydroxybenzamide (P17): A 0° C. suspension of C26 (from the previous step; 52.31 mmol) in 1,4-dioxane (20 mL) was treated with a solution of hydrogen chloride in 1,4-dioxane (4 M; 20 mL). After the reaction mixture had been allowed to stir at room temperature for 2 hours, it was concentrated in vacuo and purified via silica gel chromatography (Gradient: 0% to 100% ethyl acetate in heptane; the sample was loaded in dichloromethane containing a minimal quantity of methanol). The resulting material was partitioned between saturated aqueous sodium bicarbonate solution and ethyl acetate, whereupon the organic layer was washed with water, dried over sodium sulfate, filtered, and concentrated in vacuo. The residue was subjected to chromatography on silica gel (Gradient: 0% to 100% ethyl acetate in heptane, followed by 0% to 10% methanol in dichloromethane; the sample was loaded in dichloromethane containing a minimal quantity of methanol), providing N-{[(1r,4r)-4-(1,3-benzoxazol-2-yl)cyclohexyl]methyl}-3,5-difluoro-4-hydroxybenzamide (P17) as a white solid. Yield: 46 mg, 0.12 mmol, 5% over 2 steps. LCMS m/z 387.3 [M+H]⁺. ¹H NMR (400 MHz, methanol-d₄) δ 7.65-7.60 (m, 1H), 7.58-7.53 (m, 1H), 7.52-7.42 (m, 2H), 7.38-7.31 (m, 2H), 3.27 (d, J=7.0 Hz, 2H), 2.98 (tt, J=12.2, 3.6 Hz, 1H), 2.32-2.22 (m, 2H), 2.04-1.94 (m, 2H), 1.80-1.62 (m, 3H), 1.31-1.16 (m, 2H).

### Preparation P18

### 3,5-Difluoro-4-hydroxy-N-{[(1r,4r)-4-{3-[5-(trifluoromethyl)pyridin-2-yl]-1,2,4-oxadiazol-5-yl}cyclohexyl]methyl}benzamide (P18)

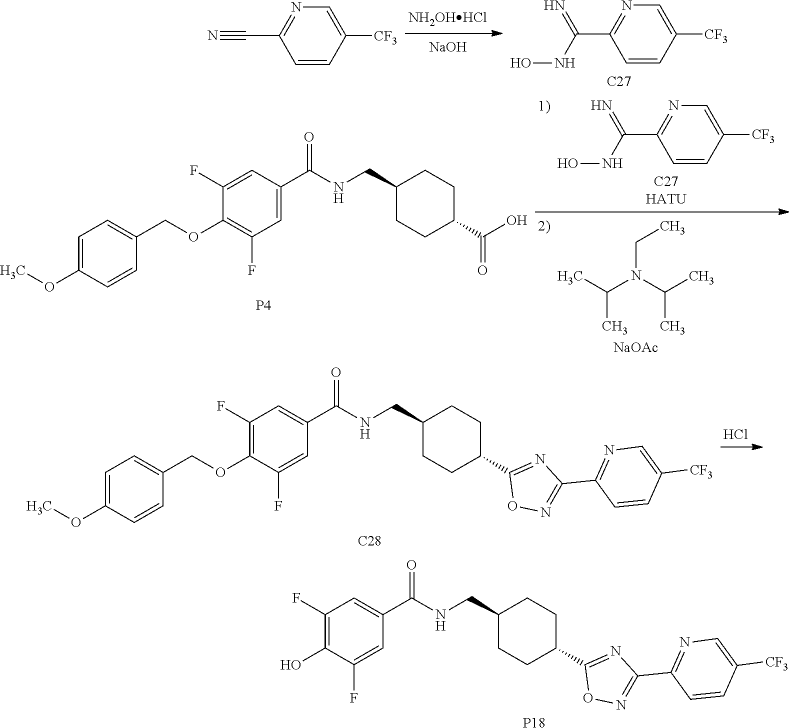

Step 1. Synthesis of N-hydroxy-5-(trifluoromethyl)pyridine-2-carboximidamide (C27): To a mixture of 5-(trifluoromethyl)pyridine-2-carbonitrile (200 mg, 1.16 mmol) and hydroxylamine hydrochloride (242 mg, 3.48 mmol) in ethanol (20 mL) was added sodium hydroxide (139 mg, 3.48 mmol). After the reaction mixture had been stirred at room temperature for 3 hours, it was concentrated in vacuo to provide C27 as a white solid. Yield: 200 mg, 0.975 mmol, 84%. LCMS m/z 206.1 [M+H]⁺.

Step 2. Synthesis of 3,5-difluoro-4-[(4-methoxyphenyl)methoxy]-N-{[(1r,4r)-4-{3-[5-(trifluoromethyl)pyridin-2-yl]-1,2,4-oxadiazol-5-yl}cyclohexyl]methyl}benzamide (C28): To a 0° C. mixture of P4 (400 mg, 0.923 mmol), N,N-diisopropylethylamine (358 mg, 2.77 mmol), and O-(7-azabenzotriazol-1-yl)-N,N,N′,N′-tetramethyluronium hexafluorophosphate (HATU; 526 mg, 1.38 mmol) in dichloromethane (20 mL) was added C27 (227 mg, 1.11 mmol). The reaction mixture was stirred at room temperature for 6 hours, whereupon it was diluted with water (20 mL) and extracted with dichloromethane (2×20 mL); the combined organic layers were washed with saturated aqueous sodium chloride solution (2×20 mL), dried over sodium sulfate, filtered, and concentrated in vacuo. Silica gel chromatography (Gradient: 0% to 7% methanol in dichloromethane) provided the acylated intermediate (100 mg, 0.161 mmol, 17%), LCMS m/z 621.3 [M+H]⁺, as a white solid.

This material was dissolved in a mixture of ethanol (4 mL) and water (1 mL), treated with sodium acetate (39.6 mg, 0.483 mmol), and stirred at 100° C. for 1 hour under microwave irradiation. After the reaction mixture had been concentrated in vacuo, it was purified using chromatography on silica gel (Gradient: 0% to 5% methanol in dichloromethane) to provide C28 as a white solid. Yield: 60 mg, 0.10 mmol, 11% from P4. LCMS m/z 603.3 [M+H]⁺.

Step 3. Synthesis of 3,5-difluoro-4-hydroxy-N-{[(1r,4r)-4-{3-[5-(trifluoromethyl)pyridin-2-yl]-1,2,4-oxadiazol-5-yl}cyclohexyl]methyl}benzamide (P18): To a solution of C28 (60 mg, 0.10 mmol) in dichloromethane (5 mL) was added a solution of hydrogen chloride in 1,4-dioxane (4 M; 1 mL). After the reaction mixture had been stirred at room temperature for 2 hours, it was concentrated in vacuo, diluted with dichloromethane (10 mL), treated with sodium bicarbonate (10 mg, 0.12 mmol), and concentrated under reduced pressure. Chromatography on silica gel (Gradient: 0% to 5% methanol in dichloromethane) afforded 3,5-difluoro-4-hydroxy-N-{[(1r,4r)-4-{3-[5-(trifluoromethyl)pyridin-2-yl]-1,2,4-oxadiazol-5-yl}cyclohexyl]methyl}benzamide (P18) as a white solid. Yield: 11.6 mg, 24.0 μmol, 24%. LCMS m/z 483.2 [M+H]⁺. ¹H NMR (400 MHz, DMSO-d₆) δ 9.19-9.14 (m, 1H), 8.48-8.39 (m, 2H), 8.27 (d, J=8.2 Hz, 1H), 7.60-7.54 (m, 2H), 3.19-3.05 (m, 3H), 2.25-2.13 (m, 2H), 1.93-1.81 (m, 2H), 1.67-1.51 (m, 3H), 1.22-1.07 (m, 2H).

### Preparation P19

### 2,3,5-Trifluoro-4-hydroxy-N-[(4-{3-[5-(trifluoromethyl)pyrimidin-2-yl]-1,2,4-oxadiazol-5-yl}bicyclo[2.2.2]octan-1-yl)methyl]benzamide (P19)

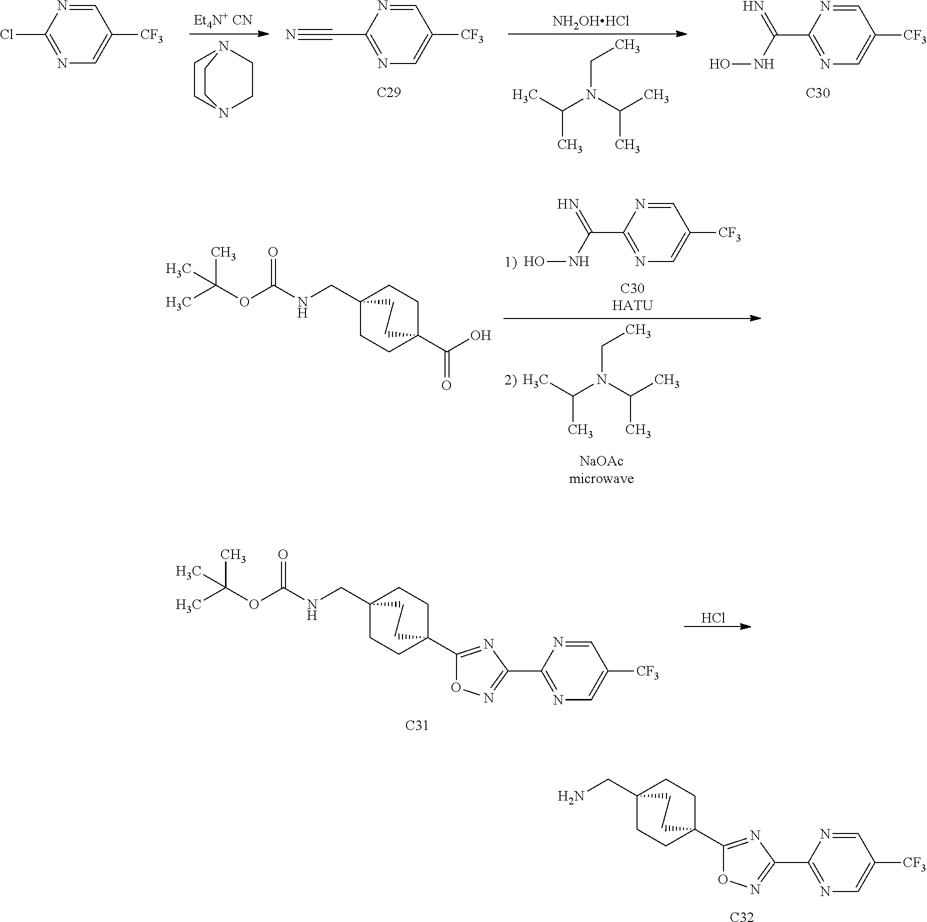

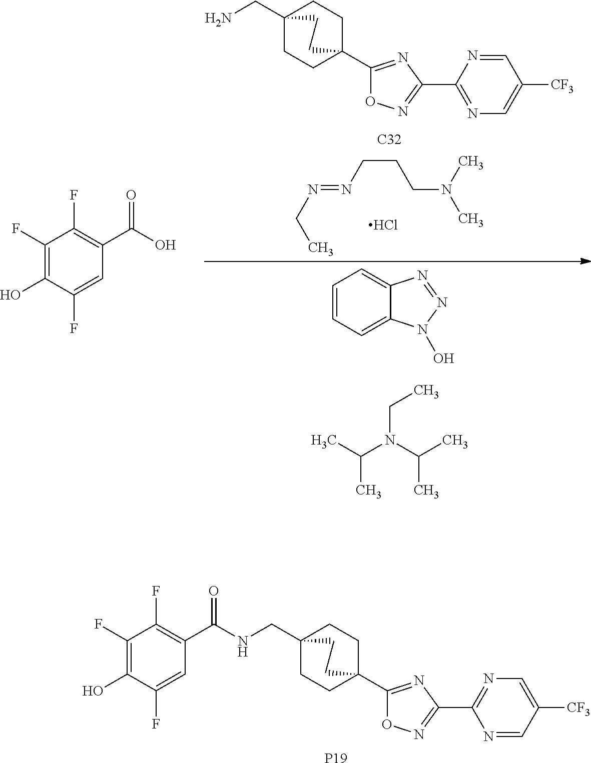

Step 1. Synthesis of 5-(trifluoromethyl)pyrimidine-2-carbonitrile (C29): A solution of tetraethylammonium cyanide (1.88 g, 12.0 mmol) and 1,4-diazabicyclo[2.2.2]octane (1.47 g, 13.1 mmol) in acetonitrile (8 mL) was added to a mixture of 2-chloro-5-(trifluoromethyl)pyrimidine (2.00 g, 11.0 mmol) in acetonitrile (8 mL), whereupon the reaction mixture was stirred at room temperature for 3 hours. Removal of solvent in vacuo provided a residue containing C29; this material, a light-yellow solid, was progressed directly to the following step.

Step 2. Synthesis of N-hydroxy-5-(trifluoromethyl)pyrimidine-2-carboximidamide (C30): A mixture of C29 (from the previous step; ≤11.0 mmol), hydroxylamine hydrochloride (1.52 g, 21.9 mmol), and N,N-diisopropylethylamine (4.26 g, 33.0 mmol) in methanol (20 mL) was stirred at 70° C. for 12 hours. Concentration of the reaction mixture in vacuo afforded C30 (1.70 g), which was taken directly into the following step. LCMS m/z 207.1 [M+H]⁺.

Step 3. Synthesis of tert-butyl [(4-{3-[5-(trifluoromethyl)pyrimidin-2-yl]-1,2,4-oxadiazol-5-yl}bicyclo[2.2.2]octan-1-yl)methyl]carbamate (C31): To a solution of C30 (from the previous step; 1.70 g, 58.25 mmol) and 4-{[(tert-butoxycarbonyl)amino]methyl}bicyclo[2.2.2]octane-1-carboxylic acid (2.57 g, 9.07 mmol) in N,N-dimethylformamide (10 mL) were added O-(7-azabenzotriazol-1-yl)-N,N,N′N′-tetramethyluronium hexafluorophosphate (HATU; 4.70 g, 12.4 mmol) and N,N-diisopropylethylamine (3.20 g, 24.8 mmol). After the reaction mixture had been stirred at 25° C. for 2 hours, it was diluted with water and filtered; the filtrate was concentrated in vacuo to provide the acyl intermediate as a yellow solid. Yield: 1.90 g, 4.03 mmol, 37% over 3 steps. LCMS m/z 472.2 [M+H]⁺.

To a solution of the acyl intermediate (2.00 g, 4.24 mmol) in a mixture of ethanol (6 mL) and water (3 mL) was added sodium acetate (1.04 g, 12.7 mmol). After the reaction mixture had been stirred at 100° C. for 1 hour under microwave irradiation, it was concentrated in vacuo. Purification via silica gel chromatography (Gradient: 0% to 6% methanol in dichloromethane) provided C31 as a white solid. Yield: 1.00 g, 2.21 mmol, 52% from the acyl intermediate. LCMS m/z 454.2 [M+H]⁺. ¹H NMR (400 MHz, methanol-d₄) δ 9.35-9.33 (m, 2H), 2.87 (s, 2H), 2.15-2.03 (m, 6H), 1.64-1.53 (m, 6H), 1.45 (s, 9H).

Step 4. Synthesis of 1-(4-{3-[5-(trifluoromethyl)pyrimidin-2-yl]-1,2,4-oxadiazol-5-yl}bicyclo[2.2.2]octan-1-yl)methanamine (C32): A solution of hydrogen chloride in 1,4-dioxane (4 M; 5 mL, 20 mmol) was added to a solution of C31 (1.00 g, 2.21 mmol) in dichloromethane (15 mL), whereupon the reaction mixture was stirred at room temperature for 2 hours. It was then concentrated in vacuo, diluted with dichloromethane (10 mL), treated with sodium bicarbonate, and again concentrated under reduced pressure. Chromatography on silica gel (Gradient: 0% to 7% methanol in dichloromethane) afforded C32 as a white solid. Yield: 800 mg, quantitative. LCMS m/z 354.2 [M+H]⁺. ¹H NMR (400 MHz, methanol-d₄) δ 9.35 (br s, 2H), 2.80 (s, 2H), 2.22-2.10 (m, 6H), 1.75-1.65 (m, 6H).

Step 5. Synthesis of 2,3,5-trifluoro-4-hydroxy-N-[(4-{3-[5-(trifluoromethyl)pyrimidin-2-yl]-1,2,4-oxadiazol-5-yl}bicyclo[2.2.2]octan-1-yl)methyl]benzamide (P19): To a solution of C32 (100 mg, 0.283 mmol) and 2,3,5-trifluoro-4-hydroxybenzoic acid (65.2 mg, 0.339 mmol) in N,N-dimethylformamide (5 mL) were added 1H-benzotriazol-1-ol (57.4 mg, 0.425 mmol), 1-[3-(dimethylamino)propyl]-3-ethylcarbodiimide hydrochloride (81.4 mg, 0.425 mmol), and N,N-diisopropylethylamine (110 mg, 0.851 mmol). After the reaction mixture had been stirred at 25° C. for 4 hours, it was diluted with water (15 mL) and extracted with dichloromethane (3×10 mL). The combined organic layers were washed with saturated aqueous sodium chloride solution (100 mL), dried over sodium sulfate, filtered, concentrated in vacuo, and purified via reversed-phase HPLC (Column: Waters XBridge C18, 19×150 mm, 5 μm; Mobile phase A: water containing 0.1% formic acid; Mobile phase B: acetonitrile; Gradient: 65% to 75% B; Flow rate: 20 mL/minute), affording 2,3,5-trifluoro-4-hydroxy-N-[(4-{3-[5-(trifluoromethyl)pyrimidin-2-yl]-1,2,4-oxadiazol-5-yl}bicyclo[2.2.2]octan-1-yl)methyl]benzamide (P19) as a white solid. Yield: 105 mg, 0.199 mmol, 70%. LCMS m/z 528.0 [M+H]⁺. ¹H NMR (400 MHz, DMSO-d₆) δ 11.36 (br s, 1H), 9.49 (s, 2H), 8.20 (br t, J=6 Hz, 1H), 7.28 (ddd, J=11.0, 6.2, 2.3 Hz, 1H), 3.09 (d, J=6.3 Hz, 2H), 2.06-1.93 (m, 6H), 1.61-1.50 (m, 6H).

### Preparation P20

### 3,5-Difluoro-4-hydroxy-N-{[(1r,4r)-4-{3-[5-(trifluoromethyl)pyrimidin-2-yl]-1,2,4-oxadiazol-5-yl}cyclohexyl]methyl}benzamide (4)

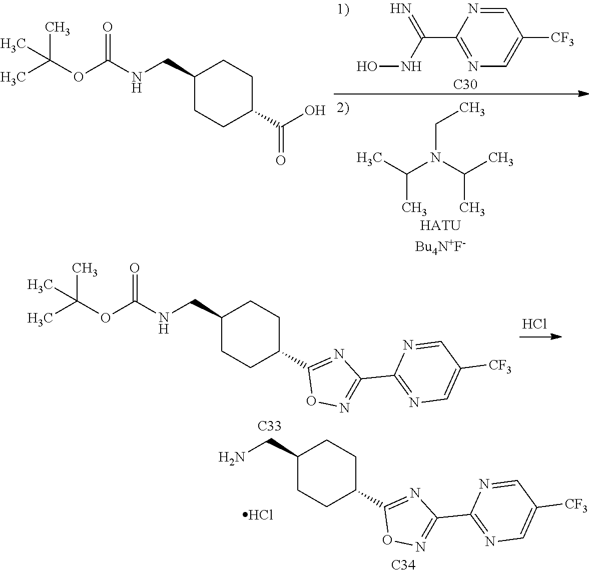

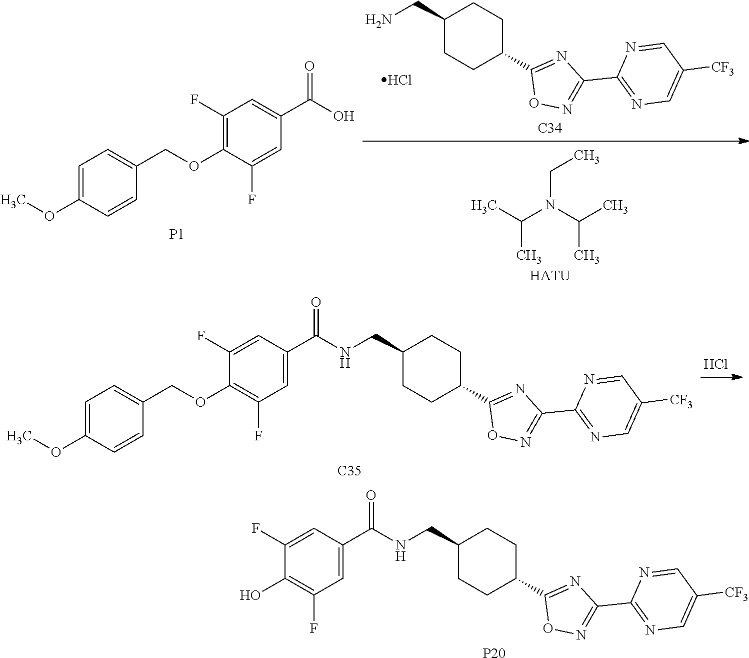

Step 1. Synthesis of tert-butyl {[(1r,4r)-4-{3-[5-(trifluoromethyl)pyrimidin-2-yl]-1,2,4-oxadiazol-5-yl}cyclohexyl]methyl}carbamate (C33): To a solution of C30 (453 mg, 2.20 mmol) in N,N-dimethylformamide (8 mL) were added (1r,4r)-4-{[(tert-butoxycarbonyl)amino]methyl}cyclohexane-1-carboxylic acid (679 mg, 2.64 mmol), O-(7-azabenzotriazol-1-yl)-N,N,N′,N′-tetramethyluronium hexafluorophosphate (HATU; 1.25 g, 3.29 mmol), and N,N-diisopropylethylamine (852 mg, 6.59 mmol). The reaction was stirred at 25° C. for 2 hours, whereupon it was diluted with ice water (30 mL) and the solid was collected via filtration, providing the acyl intermediate as a brown solid. Yield: 510 mg, 1.14 mmol, 52%. LCMS m/z 446.1 [M+H]⁺. ¹H NMR (400 MHz, chloroform-d), characteristic peaks, integrations are approximate: δ 9.08 (br s, 2H), 3.05-2.96 (m, 2H), 2.48 (tt, J=12.3, 3.6 Hz, 1H), 2.16-2.06 (m, 2H), 1.92-1.82 (m, 2H), 1.66-1.53 (m, 2H), 1.09-0.94 (m, 2H).

To a solution of the acyl intermediate (700 mg, 1.57 mmol) in dichloromethane (5 mL) was added a solution of tetrabutylammonium fluoride in tetrahydrofuran (1 M; 5 mL, 5 mmol), whereupon the reaction mixture was stirred at 25° C. for 4 hours. It was then concentrated in vacuo and subjected to chromatography on silica gel (Gradient: 0% to 50% ethyl acetate in petroleum ether), affording C33 as a white solid. Yield: 340 mg, 0.795 mmol, 51%. LCMS m/z 450.1 [M+Na⁺]. ¹H NMR (400 MHz, chloroform-d) δ 9.20-9.18 (m, 2H), 4.60 (br s, 1H), 3.11-2.99 (m, 3H), 2.34-2.24 (m, 2H), 2.00-1.91 (m, 2H), 1.84-1.69 (m, 2H), 1.6-1.50 (m, 1H, assumed; largely obscured by water peak), 1.45 (s, 9H), 1.20-1.06 (m, 2H).

Step 2. Synthesis of 1-[(1r,4r)-4-{3-[5-(trifluoromethyl)pyrimidin-2-yl]-1,2,4-oxadiazol-5-yl}cyclohexyl]methanamine, hydrochloride salt (C34): A solution of hydrogen chloride in 1,4-dioxane (4 M; 2 mL, 8 mmol) was added to a solution of C33 (340 mg, 0.795 mmol) in dichloromethane (5 mL). After the reaction mixture had been stirred at 25° C. for 2 hours, it was concentrated in vacuo to provide C34 as a white solid. Yield: 200 mg, 0.550 mmol, 69%. LCMS m/z 328.1 [M+H]⁺. ¹H NMR (400 MHz, DMSO-d₆) δ 9.51-9.49 (m, 2H), 8.00 (br s, 3H), 3.14 (tt, J=12.1, 3.6 Hz, 1H), 2.75-2.65 (m, 2H), 2.27-2.17 (m, 2H), 1.97-1.87 (m, 2H), 1.73-1.53 (m, 3H), 1.23-1.09 (m, 2H).

Step 3. Synthesis of 3,5-difluoro-4-[(4-methoxyphenyl)methoxy]-N-{[(1r,4r)-4-{3-[5-(trifluoromethyl)pyrimidin-2-yl]-1,2,4-oxadiazol-5-yl}cyclohexyl]methyl}benzamide (C35): To a 0° C. solution of P1 (27 mg, 91.8 μmol), C34 (30 mg, 82 μmol), and O-(7-azabenzotriazol-1-yl)-N,N,N,N′-tetramethyluronium hexafluorophosphate (HATU; 52.3 mg, 0.138 mmol) in N,N-dimethylformamide (3 mL) was added N,N-diisopropylethylamine (35.5 mg, 0.275 mmol), whereupon the reaction mixture was stirred at 25° C. for 2 hours. It was then treated with ice water (30 mL) and the resulting solid was collected via filtration to provide C35 as a white solid (60 mg). This material was taken directly to the following step. LCMS m/z 626.2 [M+Na⁺]. ¹H NMR (400 MHz, DMSO-d₆) δ 9.49 (s, 2H), 8.56 (br t, J=5.8 Hz, 1H), 7.66-7.56 (m, 2H), 7.34 (d, J=8.5 Hz, 2H), 6.92 (d, J=8.4 Hz, 2H), 5.17 (s, 2H), 3.74 (s, 3H), 3.19-3.08 (m, 3H), 2.24-2.15 (m, 2H), 1.91-1.82 (m, 2H), 1.67-1.51 (m, 3H), 1.22-1.08 (m, 2H).

Step 4. Synthesis of 3,5-difluoro-4-hydroxy-N-{[(1r,4r)-4-{3-[5-(trifluoromethyl)pyrimidin-2-yl]-1,2,4-oxadiazol-5-yl}cyclohexyl]methyl}benzamide (P20): A solution of C35 (from the previous step; 60 mg, 582 μmol) in dichloromethane (4 mL) was treated with a solution of hydrogen chloride in 1,4-dioxane (4 M; 2 mL), and the reaction mixture was stirred at 25° C. for 2 hours. After removal of volatiles in vacuo, the residue was purified using silica gel chromatography (Gradient: 0% to 10% methanol in dichloromethane) to afford 3,5-difluoro-4-hydroxy-N-{[(1r,4r)-4-{3-[5-(trifluoromethyl)pyrimidin-2-yl]-1,2,4-oxadiazol-5-yl}cyclohexyl]methyl}benzamide (P20) as a white solid. Yield: 14.3 mg, 29.6 μmol, 36% over 2 steps. LCMS m/z 484.1 [M+H]⁺. ¹H NMR (400 MHz, DMSO-d₆) δ 10.84 (br s, 1H), 9.49 (s, 2H), 8.44 (br t, J=5.8 Hz, 1H), 7.64-7.52 (m, 2H), 3.20-3.07 (m, 3H), 2.25-2.15 (m, 2H), 1.93-1.82 (m, 2H), 1.67-1.51 (m, 3H), 1.22-1.08 (m, 2H).

### Preparation P21

### 2,3,5-Trifluoro-4-hydroxy-N-({(1r,4r)-4-[6-(1-methyl-1H-pyrazol-4-yl)-2H-indazol-2-yl]cyclohexyl}methyl)benzamide, trifluoroacetate salt (P21)

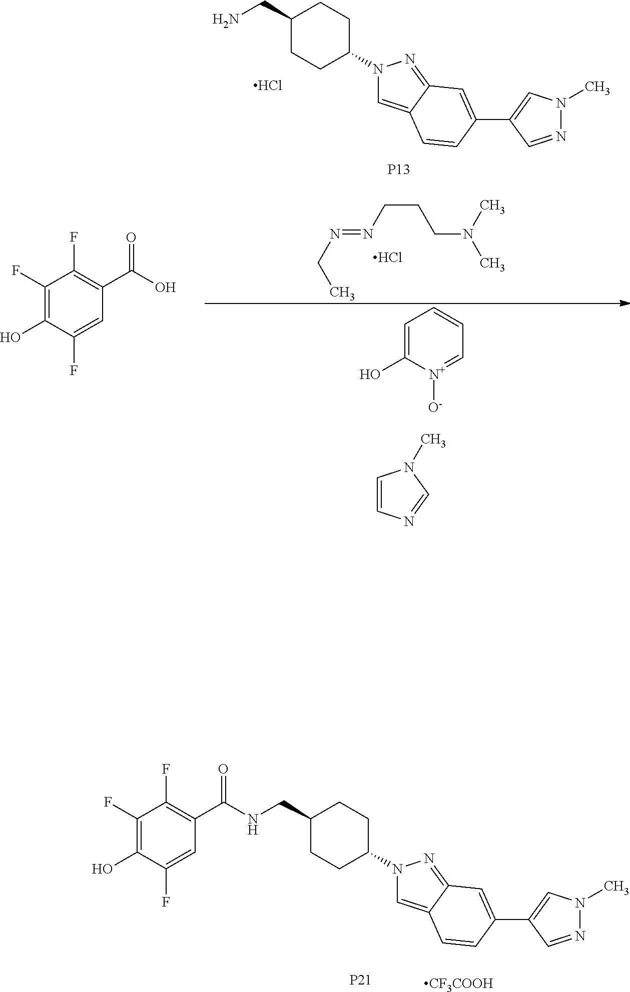

A mixture of 2,3,5-trifluoro-4-hydroxybenzoic acid (50 mg, 0.26 mmol), P13 (75 mg, 0.22 mmol), 2-hydroxypyridine 1-oxide (26.5 mg, 0.239 mmol), and 1-methyl-1H-imidazole (52 μL, 0.65 mmol) in a mixture of water (0.32 mL) and N,N-dimethylformamide (1.3 mL) was allowed to stir at room temperature for 5 minutes, whereupon 1-[3-(dimethylamino)propyl]-3-ethylcarbodiimide hydrochloride (45.7 mg, 0.238 mmol) was added in one portion, and the reaction mixture was stirred at room temperature overnight. After dilution with water, the reaction mixture was acidified by addition of 1 M hydrochloric acid, and extracted with ethyl acetate. The organic layer was washed with water, dried over magnesium sulfate, filtered, and concentrated in vacuo to afford a solid, which was purified via reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 5% to 95% B over 8.54 minutes, followed by 95% B for 1.46 minutes; Flow rate: 25 mL/minute) to provide 2,3,5-trifluoro-4-hydroxy-N-({(1r,4r)-4-[6-(1-methyl-1H-pyrazol-4-yl)-2H-indazol-2-yl]cyclohexyl}methyl)benzamide, trifluoroacetate salt (P21). Yield: 46.3 mg, 95.8 μmol, 44%. LCMS m/z 484.6 [M+H]⁺. Retention time: 2.51 minutes (Analytical conditions. Column: Waters Atlantis dC18, 4.6×50 mm, 5 μm; Mobile phase A: water containing 0.05% trifluoroacetic acid (v/v); Mobile phase B: acetonitrile containing 0.05% trifluoroacetic acid (v/v); Gradient: 5.0% to 95% B over 4.0 minutes, then 95% B for 1.0 minute; Flow rate: 2 mL/minute).

### Preparation P22

### 3,5-Difluoro-4-hydroxy-N-{[(1r,4r)-4-(5-methoxy-2H-pyrazolo[3,4-c]pyridin-2-yl)cyclohexyl]methyl}benzamide (P22)

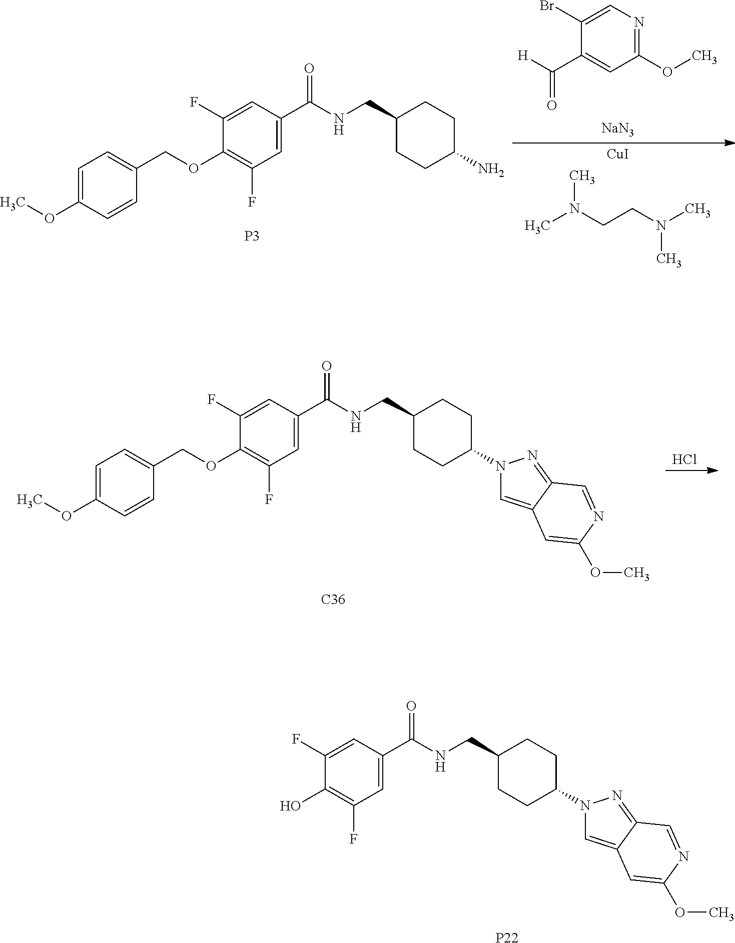

Step 1. Synthesis of 3,5-difluoro-4-[(4-methoxyphenyl)methoxy]-N-{[(1r,4r)-4-(5-methoxy-2H-pyrazolo[3,4-c]pyridin-2-yl)cyclohexyl]methyl}benzamide (C36): A mixture of P3 (134 mg, 0.331 mmol) and 5-bromo-2-methoxypyridine-4-carbaldehyde (65 mg, 0.30 mmol) in toluene (8 mL) was stirred at 90° C. for 8 hours. The reaction mixture was then concentrated under reduced pressure and diluted with dimethyl sulfoxide (8 mL); to this were added copper(I) iodide (5.71 mg, 30.0 μmol), N¹,N¹,N²,N²-tetramethylethane-1,2-diamine (3.49 mg, 30.0 μmol), and sodium azide (39.1 mg, 0.601 mmol). After this reaction mixture had been stirred at 100° C. for 8 hours, it was treated with water (20 mL) and extracted with ethyl acetate (2×20 mL). The combined organic layers were washed with saturated aqueous sodium chloride solution (2×20 mL), dried over sodium sulfate, filtered, concentrated in vacuo, and purified via silica gel chromatography (Gradient: 0% to 8% methanol in dichloromethane) to provide C36 as a brown solid. Yield: 50 mg, 93 μmol, 31%. LCMS m/z 537.3 [M+H]⁺.

Step 2. Synthesis of 3,5-difluoro-4-hydroxy-N-{[(1r,4r)-4-(5-methoxy-2H-pyrazolo[3,4-c]pyridin-2-yl)cyclohexyl]methyl}benzamide (P22): To a solution of C36 (50 mg, 93 μmol) in dichloromethane (5 mL) was added a solution of hydrogen chloride in 1,4-dioxane (4 M; 1 mL). The reaction mixture was stirred at room temperature for 2 hours, whereupon it was concentrated under reduced pressure, treated with dichloromethane (10 mL) and sodium bicarbonate (10 mg), and again concentrated in vacuo. Silica gel chromatography (Gradient: 0% to 7% methanol in dichloromethane) provided 3,5-difluoro-4-hydroxy-N-{[(1r,4r)-4-(5-methoxy-2H-pyrazolo[3,4-c]pyridin-2-yl)cyclohexyl]methyl}benzamide (P22) as a white solid. Yield: 5.1 mg, 12 μmol, 13%. LCMS m/z 417.2 [M+H]⁺. ¹H NMR (400 MHz, DMSO-d₆) δ 10.84 (s, 1H), 8.92-8.87 (m, 1H), 8.47 (br t, J=5.8 Hz, 1H), 8.37 (s, 1H), 7.64-7.53 (m, 2H), 6.88 (d, J=1.2 Hz, 1H), 4.59-4.49 (m, 1H), 3.84 (s, 3H), 3.17 (dd, J=6, 6 Hz, 2H), 2.21-2.10 (m, 2H), 1.99-1.83 (m, 4H), 1.74-1.60 (m, 1H), 1.29-1.14 (m, 2H).

### Preparation P23

### 2,3,5-Trifluoro-4-hydroxy-N-({4-[6-(pyrimidin-2-yl)-2H-indazol-2-yl]bicyclo[2.2.2]octan-1-yl}methyl)benzamide (P23)

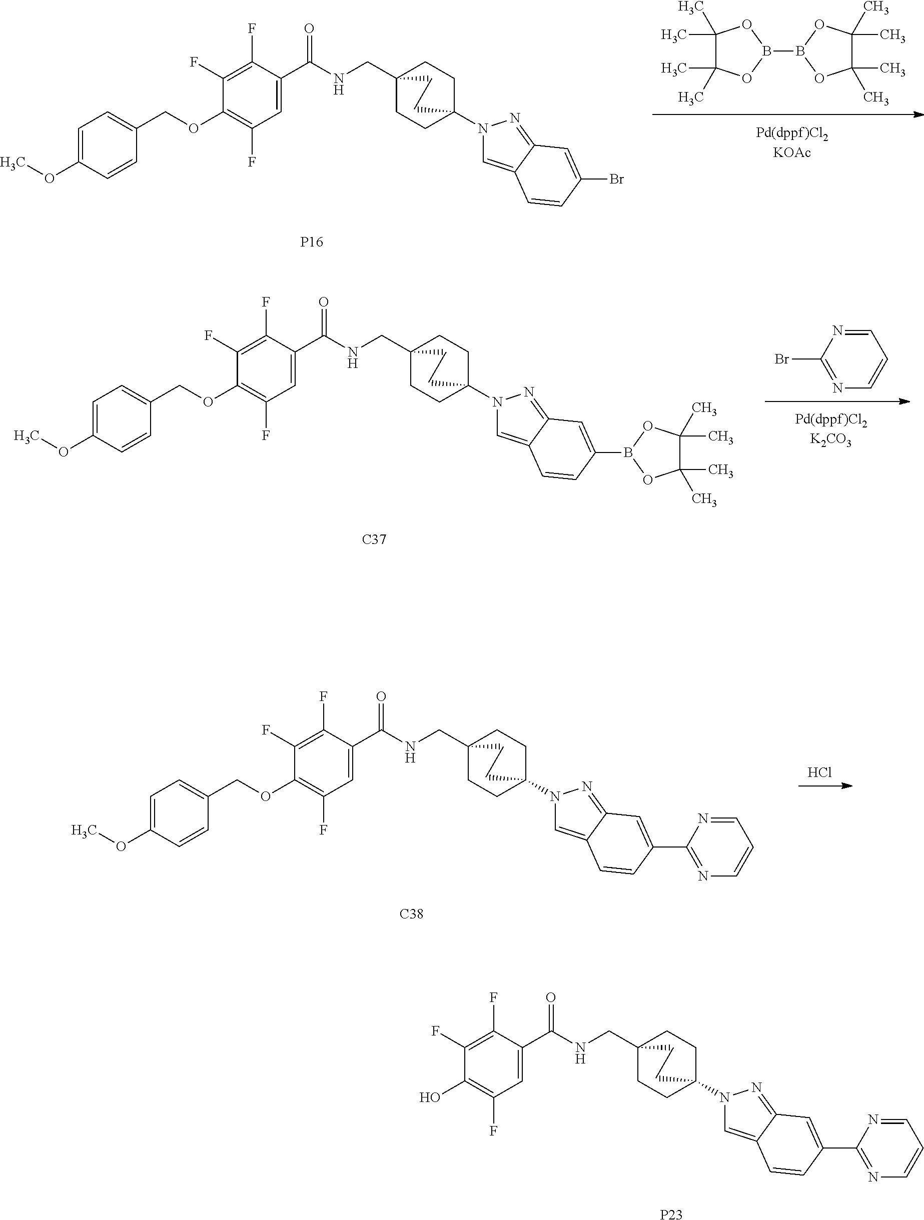

Step 1. Synthesis of 2,3,5-trifluoro-4-[(4-methoxyphenyl)methoxy]-N-({4-[6-(4,4,5,5-tetramethyl-1,3,2-dioxaborolan-2-yl)-2H-indazol-2-yl]bicyclo[2.2.2]octan-1-yl}methyl)benzamide (C37): To a solution of P16 (100 mg, 0.159 mmol) and 4,4,4′,4′,5,5,5′,5′-octamethyl-2,2′-bi-1,3,2-dioxaborolane (48.5 mg, 0.191 mmol) in 1,4-dioxane (5 mL) were added [1,1′-bis(diphenylphosphino)ferrocene]dichloropalladium(II) (11.6 mg, 15.9 μmol) and potassium acetate (46.8 mg, 0.477 mmol), whereupon the reaction mixture was stirred at 90° C. for 12 hours. Concentration in vacuo provided C37, which was progressed directly to the following step.

Step 2. Synthesis of 2,3,5-trifluoro-4-[(4-methoxyphenyl)methoxy]-N-({4-[6-(pyrimidin-2-yl)-2H-indazol-2-yl]bicyclo[2.2.2]octan-1-yl}methyl)benzamide (C38): To a solution of C37 (from the previous step; 50.159 mmol) and 2-bromopyrimidine (28.2 mg, 0.177 mmol) in 1,4-dioxane (5 mL) were added [1,1′-bis(diphenylphosphino)ferrocene]dichloropalladium(II) (10.8 mg, 14.8 μmol) and potassium carbonate (61.4 mg, 0.444 mmol). After the reaction mixture had been stirred at 90° C. for 12 hours, it was concentrated in vacuo and purified via silica gel chromatography (Gradient: 0% to 60% ethyl acetate in petroleum ether), affording C38 as a white solid. Yield: 40 mg, 64 μmol, 40% over 2 steps. LCMS m/z 628.2 [M+H]⁺.

Step 3. Synthesis of 2,3,5-trifluoro-4-hydroxy-N-({4-[6-(pyrimidin-2-yl)-2H-indazol-2-yl]bicyclo[2.2.2]octan-1-yl}methyl)benzamide (P23): To a solution of C38 (40 mg, 64 μmol) in dichloromethane (10 mL) was added a solution of hydrogen chloride in 1,4-dioxane (4 M; 2 mL, 8 mmol), and the reaction mixture was stirred at 25° C. for 2 hours. It was then concentrated in vacuo, treated with dichloromethane (10 mL) and sodium bicarbonate (1 g), and concentrated under reduced pressure. Chromatography on silica gel (Gradient: 0% to 7% methanol in dichloromethane) provided 2,3,5-trifluoro-4-hydroxy-N-({4-[6-(pyrimidin-2-yl)-2H-indazol-2-yl]bicyclo[2.2.2]octan-1-yl}methyl)benzamide (P23) as a white solid. Yield: 6.0 mg, 12 μmol, 19%. LCMS m/z 508.1 [M+H]⁺. ¹H NMR (400 MHz, DMSO-d₆) δ 11.38 (br s, 1H), 8.91 (d, J=4.8 Hz, 2H), 8.67-8.65 (m, 1H), 8.47 (d, J=1.0 Hz, 1H), 8.21 (br t, J=6 Hz, 1H), 8.08 (dd, J=8.8, 1.4 Hz, 1H), 7.79 (dd, J=8.8, 0.9 Hz, 1H), 7.43 (t, J=4.8 Hz, 1H), 7.29 (ddd, J=11.1, 6.2, 2.3 Hz, 1H), 3.12 (d, J=6.3 Hz, 2H), 2.25-2.15 (m, 6H), 1.74-1.63 (m, 6H).

### Preparation P24

### 3,5-Difluoro-4-hydroxy-N-({(1r,4r)-4-[6-(2-methoxypyrimidin-5-yl)-2H-pyrazolo[4,3-c]pyridin-2-yl]cyclohexyl}methyl)benzamide (P24)

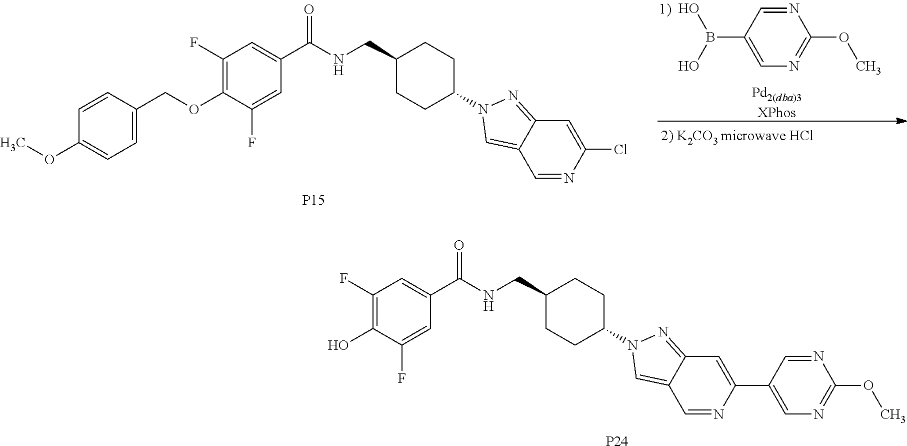

To a solution of P15 (60 mg, 0.11 mmol), (2-methoxypyrimidin-5-yl)boronic acid (25.6 mg, 0.166 mmol), 2-dicyclohexylphosphino-2′,4′,6′-triisopropylbiphenyl (XPhos; 21.1 mg, 44.3 μmol), and potassium carbonate (46.0 mg, 0.333 mmol) in a mixture of 1,4-dioxane (2 mL) and water (0.4 mL) was added tris(dibenzylideneacetone)dipalladium(0) (20.3 mg, 22.2 μmol). The reaction mixture was stirred at 100° C. for 1 hour under microwave irradiation, whereupon it was concentrated under reduced pressure. The residue was dissolved in dichloromethane (4 mL), treated with a solution of hydrogen chloride in 1,4-dioxane (4 M; 1 mL, 4 mmol), and stirred at 15° C. for 1 hour. After solvents had been removed in vacuo, purification via reversed-phase HPLC (Column: Welch Xtimate C18, 30×250 mm, 10 μm; Mobile phase A: water containing 0.05% formic acid; Mobile phase B: acetonitrile; Gradient: 43% to 95% B; Flow rate: 50 mL/minute) afforded 3,5-difluoro-4-hydroxy-N-({(1r,4r)-4-[6-(2-methoxypyrimidin-5-yl)-2H-pyrazolo[4,3-c]pyridin-2-yl]cyclohexyl}methyl)benzamide (P24) as a solid. Yield: 5.2 mg, 10 μmol, 9%. LCMS m/z 495.1 [M+H]⁺. ¹H NMR (400 MHz, DMSO-d₆) δ 9.30 (s, 2H), 9.25 (d, J=1.3 Hz, 1H), 8.76 (br s, 1H), 8.43 (br s, 1H), 8.23-8.20 (m, 1H), 7.62-7.49 (m, 2H), 4.64-4.50 (m, 1H), 3.98 (s, 3H), 3.18 (dd, J=6, 6 Hz, 2H), 2.24-2.12 (m, 2H), 2.01-1.84 (m, 4H), 1.75-1.60 (m, 1H), 1.33-1.12 (m, 2H).

### Preparation P25

### 2,3,5-Trifluoro-4-hydroxy-N-({(1r,4r)-4-[6-(pyrimidin-5-yl)-2H-indazol-2-yl]cyclohexyl}methyl)benzamide (P25)

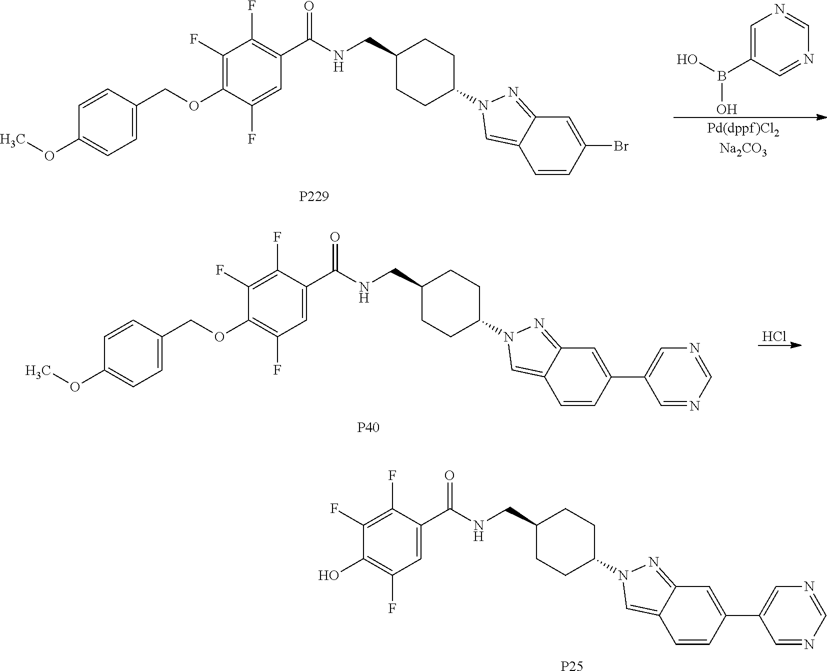

Step 1. Synthesis of 2,3,5-trifluoro-4-[(4-methoxyphenyl)methoxy]-N-({(1r,4r)-4-[6-(pyrimidin-5-yl)-2H-indazol-2-yl]cyclohexyl}methyl)benzamide (C40): A mixture of P229 (700 mg, 1.16 mmol), pyrimidin-5-ylboronic acid (144 mg, 1.16 mmol), sodium carbonate (369 mg, 3.48 mmol), and [1,1′-bis(diphenylphosphino)ferrocene]dichloropalladium(II), dichloromethane complex (94.2 mg, 0.115 mmol) in a mixture of 1,4-dioxane (20 mL) and water (5 mL) was stirred at 90° C. for 16 hours. The reaction mixture was then concentrated in vacuo and purified via silica gel chromatography (Gradient: 0% to 100% ethyl acetate in petroleum ether), providing C40 as a white solid. Yield: 300 mg, 0.499 mmol, 43%. LCMS m/z 602.2 [M+H]⁺. ¹H NMR (400 MHz, DMSO-d₆) δ 9.20 (s, 2H), 9.18 (s, 1H), 8.53-8.46 (m, 2H), 8.08 (br s, 1H), 7.85 (d, J=8.7 Hz, 1H), 7.45 (dd, J=8.7, 1.6 Hz, 1H), 7.41-7.34 (m, 1H), 7.35 (d, J=8.6 Hz, 2H), 6.94 (d, J=8.6 Hz, 2H), 5.22 (s, 2H), 4.59-4.45 (m, 1H), 3.75 (s, 3H), 3.18 (dd, J=6, 6 Hz, 2H), 2.23-2.13 (m, 2H), 2.01-1.86 (m, 4H), 1.74-1.60 (m, 1H), 1.32-1.16 (m, 2H).

Step 2. Synthesis of 2,3,5-trifluoro-4-hydroxy-N-({(1r,4r)-4-[6-(pyrimidin-5-yl)-2H-indazol-2-yl]cyclohexyl}methyl)benzamide (P25): To a suspension of C40 (300 mg, 0.499 mmol) in dichloromethane (4.0 mL) was added a solution of hydrogen chloride in 1,4-dioxane (4 M; 1 mL, 4 mmol). The reaction mixture was stirred at 25° C. for 2 hours, whereupon it was concentrated in vacuo and purified using silica gel chromatography (Gradient: 0% to 100% ethyl acetate in petroleum ether) to afford 2,3,5-trifluoro-4-hydroxy-N-({(1r,4r)-4-[6-(pyrimidin-5-yl)-2H-indazol-2-yl]cyclohexyl}methyl)benzamide (P25) as a white solid. Yield: 200 mg, 0.415 mmol, 83%. LCMS m/z 482.2 [M+H]⁺. ¹H NMR (400 MHz, DMSO-d₆) δ 11.41 (br s, 1H), 9.21 (s, 2H), 9.18 (s, 1H), 8.49 (s, 1H), 8.37-8.30 (m, 1H), 8.09 (br s, 1H), 7.85 (d, J=8.7 Hz, 1H), 7.45 (dd, J=8.7, 1.6 Hz, 1H), 7.30 (ddd, J=11.1, 6.3, 2.2 Hz, 1H), 4.58-4.46 (m, 1H), 3.21-3.14 (m, 2H), 2.23-2.13 (m, 2H), 2.02-1.86 (m, 4H), 1.75-1.61 (m, 1H), 1.33-1.16 (m, 2H).

### Preparation P26

### N-{[(1r,4r)-4-{6-[1-(2,2-Difluoroethyl)-1H-pyrazol-4-yl]-2H-indazol-2-yl}cyclohexyl]methyl}-3,5-difluoro-4-hydroxybenzamide, trifluoroacetate salt (P26)

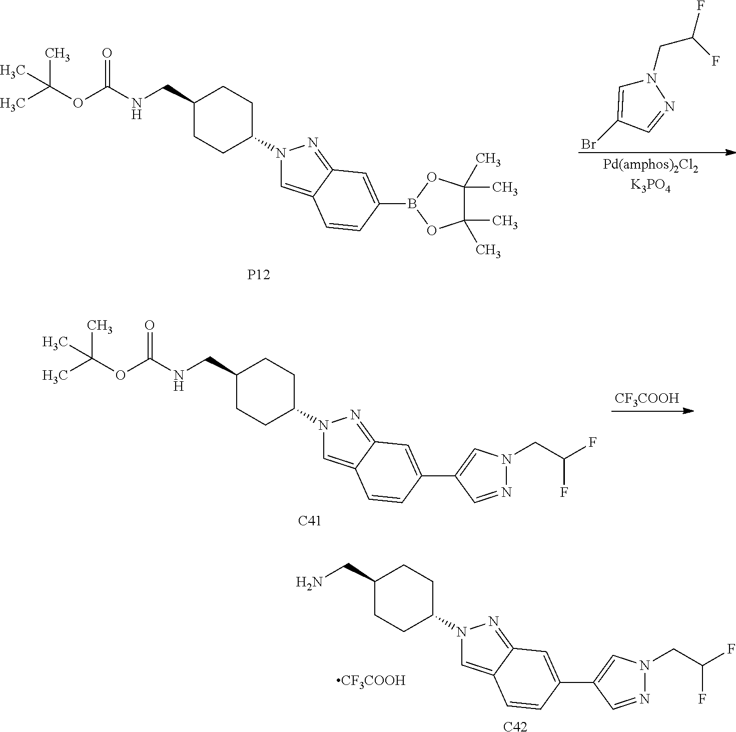

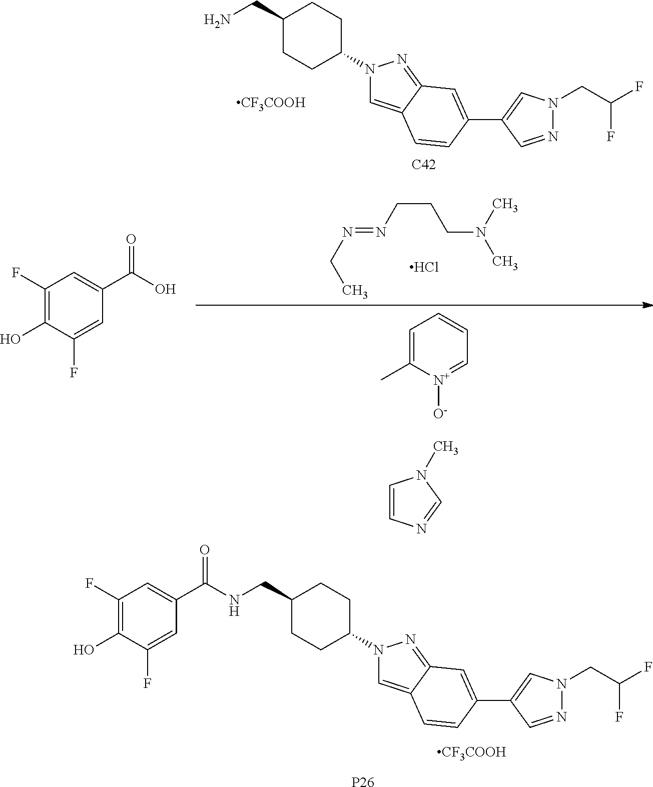

Step 1. Synthesis of tert-butyl {[(1r,4r)-4-{6-[1-(2,2-difluoroethyl)-1H-pyrazol-4-yl]-2H-indazol-2-yl}cyclohexyl]methyl}carbamate (C41): To a mixture of 4-bromo-1-(2,2-difluoroethyl)-1H-pyrazole (46.1 mg, 0.218 mmol) and P12 (100 mg, 0.220 mmol) were added 1,4-dioxane (1.8 mL) and water (0.6 mL), followed by tripotassium phosphate (140 mg, 0.660 mmol) and bis[di-tert-butyl(4-dimethylaminophenyl)phosphine]dichloropalladium(II) [Pd(amphos)₂Cl₂; 15.5 mg, 21.9 μmol]. The reaction mixture was heated at 85° C. for 18 hours, whereupon it was partitioned between water and ethyl acetate. After the aqueous layer had been extracted twice with ethyl acetate, the combined organic layers were dried over magnesium sulfate, filtered, and concentrated in vacuo. Silica gel chromatography (Gradient: 0% to 7.5% methanol in dichloromethane) provided C41 as an oil. Yield: 80 mg, 0.17 mmol, 78%. LCMS m/z 460.3 [M+H]⁺. ¹H NMR (400 MHz, chloroform-d), characteristic peaks: δ 7.91 (br s, 1H), 7.89 (s, 1H), 7.79 (br s, 1H), 7.74 (s, 1H), 7.65 (d, J=8.7 Hz, 1H), 7.22 (dd, J=8.7, 1.4 Hz, 1H), 6.13 (tt, J=55.4, 4.3 Hz, 1H), 4.68-4.58 (m, 1H), 4.51 (td, J=13.5, 4.3 Hz, 2H), 4.43-4.32 (m, 1H), 3.06 (dd, J=6, 6 Hz, 2H), 2.39-2.28 (m, 2H), 1.46 (s, 9H).

Step 2. Synthesis of 1-[(1r,4r)-4-{6-[1-(2,2-difluoroethyl)-1H-pyrazol-4-yl]-2H-indazol-2-yl}cyclohexyl]methanamine, trifluoroacetate salt (C42): Trifluoroacetic acid (0.5 mL, 6 mmol) was added drop-wise to a solution of C41 (80 mg, 0.17 mmol) in dichloromethane (2 mL). The reaction mixture was stirred for 30 minutes at room temperature, whereupon it was concentrated in vacuo to dryness; the residue was azeotroped twice with dichloromethane, providing C42 as a colorless oil (84 mg), most of which was taken directly to the following step. LCMS m/z 360.3 [M+H]⁺. ¹H NMR (400 MHz, methanol-d₄), characteristic peaks: δ 8.28-8.26 (m, 1H), 8.13 (s, 1H), 7.98 (s, 1H), 7.75 (br s, 1H), 7.72 (d, J=8.7 Hz, 1H), 7.38-7.33 (m, 1H), 6.22 (tt, J=55.2, 3.9 Hz, 1H), 4.61 (td, J=14.4, 3.9 Hz, 2H), 4.56-4.44 (m, 1H), 2.90 (d, J=7.0 Hz, 2H), 2.37-2.27 (m, 2H), 2.13-2.00 (m, 4H), 1.89-1.75 (m, 1H).

Step 3. Synthesis of N-{[(1r,4r)-4-{6-[1-(2,2-difluoroethyl)-1H-pyrazol-4-yl]-2H-indazol-2-yl}cyclohexyl]methyl}-3,5-difluoro-4-hydroxybenzamide, trifluoroacetate salt (P26): A solution of C42 (from the previous step; 82 mg, 50.17 mmol) in N,N-dimethylformamide (1.8 mL) was treated with water (0.4 mL). 3,5-Difluoro-4-hydroxybenzoic acid (36.2 mg, 0.208 mmol), 1-methyl-1H-imidazole (41.4 μL, 0.520 mmol), and 2-hydroxypyridine 1-oxide (64 mg, 5.76 mmol) were sequentially added, and the reaction mixture was stirred for 20 minutes at room temperature. 1-[3-(Dimethylamino)propyl]-3-ethylcarbodiimide hydrochloride (98%, 33.9 mg, 0.173 mmol) was then added, and stirring was continued for 18 hours at room temperature. The reaction mixture was diluted with water (10 mL), and acidified to pH 4 by addition of 1 M hydrochloric acid, whereupon it was extracted 3 times with ethyl acetate. The combined organic layers were washed 5 times with water, dried over magnesium sulfate, filtered, and concentrated in vacuo; reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 20% to 60% B over 8.5 minutes, then 60% to 95% B over 0.5 minutes; Flow rate: 25 mL/minute) afforded N-{[(1r,4r)-4-{6-[1-(2,2-difluoroethyl)-1H-pyrazol-4-yl]-2H-indazol-2-yl}cyclohexyl]methyl}-3,5-difluoro-4-hydroxybenzamide, trifluoroacetate salt (P26). Yield: 29.3 mg, 46.5 μmol, 27% over 2 steps. LCMS m/z 516.5 [M+H]⁺. Retention time: 2.59 minutes (Analytical conditions. Column: Waters Atlantis dC18, 4.6×50 mm, 5 μm; Mobile phase A: water containing 0.05% trifluoroacetic acid (v/v); Mobile phase B: acetonitrile containing 0.05% trifluoroacetic acid (v/v); Gradient: 5.0% to 95% B over 4.0 minutes, then 95% B for 1.0 minute; Flow rate: 2 mL/minute).

### Preparation P27

### 2,3,5-Trifluoro-4-hydroxy-N-{[(1r,4r)-4-{6-[4-(trifluoromethyl)-1H-pyrazol-1-yl]-2H-indazol-2-yl}cyclohexyl]methyl}benzamide, trifluoroacetate salt (P27)

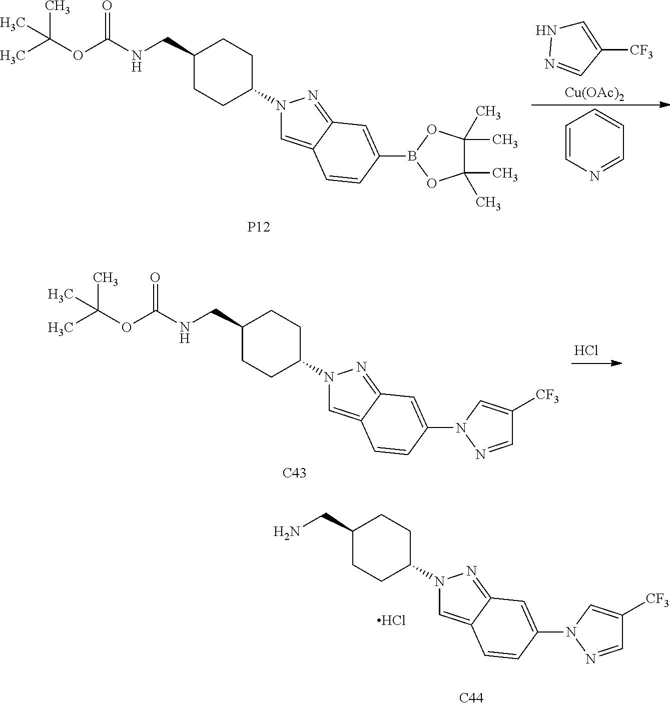

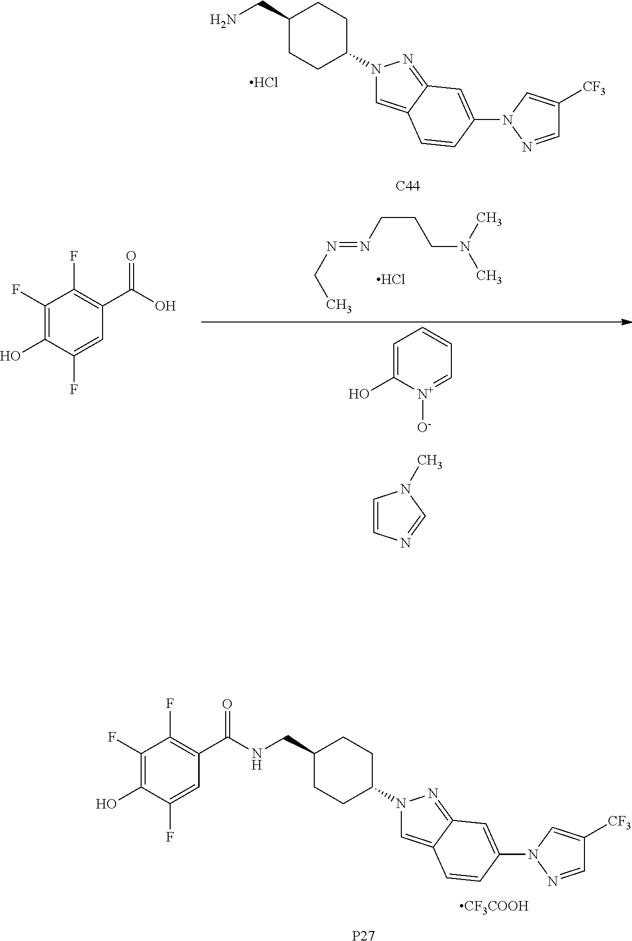

Step 1. Synthesis of tert-butyl {[(1r,4r)-4-{6-[4-(trifluoromethyl)-1H-pyrazol-1-yl]-2H-indazol-2-yl}cyclohexyl]methyl}carbamate (C43): A mixture of P12 (100 mg, 0.220 mmol), 4-(trifluoromethyl)-1H-pyrazole (120 mg, 0.882 mmol), and copper(II) acetate (53 mg, 0.29 mmol) in pyridine (1.3 mL) was heated at 90° C. for 18 hours. For the first hour, the reaction mixture was left open to the air; it was subsequently capped, a needle was inserted through the cap to the atmosphere, and heating was continued for an additional 17 hours. The reaction mixture was concentrated in vacuo to dryness and the residue was partitioned between dichloromethane and water. The organic layer was subjected to silica gel chromatography (Gradient: 0% to 7.5% methanol in dichloromethane), affording C43 as a colorless oil. Yield: 80.0 mg, 0.173 mmol, 79%. LCMS m/z 464.3 [M+H]⁺. ¹H NMR (400 MHz, chloroform-d), characteristic peaks: δ 8.21 (br s, 1H), 8.00 (br s, 1H), 7.92 (s, 1H), 7.91-7.89 (m, 1H), 7.77 (br d, J=9.0 Hz, 1H), 7.49 (dd, J=9.0, 1.9 Hz, 1H), 4.64 (br s, 1H), 4.42 (tt, J=11.9, 3.7 Hz, 1H), 3.08 (dd, J=6, 6 Hz, 2H), 2.40-2.28 (m, 2H), 2.07-1.89 (m, 4H), 1.70-1.55 (m, 1H), 1.46 (s, 9H).

Step 2. Synthesis of 1-[(1r,4r)-4-{6-[4-(trifluoromethyl)-1H-pyrazol-1-yl]-2H-indazol-2-yl}cyclohexyl]methanamine, hydrochloride salt (C44): To a solution of C43 (80.0 mg, 0.173 mmol) in 1,4-dioxane (2 mL) was added a solution of hydrogen chloride in 1,4-dioxane (4 M; 1 mL, 4 mmol), whereupon the reaction mixture was stirred for 2 hours. Removal of solvent in vacuo provided a residue that was azeotroped twice with dichloromethane to afford C44 as a white solid (75 mg); most of this material was taken directly to the following step. LCMS m/z 364.3 [M+H]⁺.

Step 3. Synthesis of 2,3,5-trifluoro-4-hydroxy-N-{[(1r,4r)-4-{6-[4-(trifluoromethyl)-1H-pyrazol-1-yl]-2H-indazol-2-yl}cyclohexyl]methyl}benzamide, trifluoroacetate salt (P27): A solution of C44 (from the previous step; 69 mg, 50.16 mmol) in N,N-dimethylformamide (1.8 mL) was treated with water (0.4 mL), whereupon the following reagents were sequentially added: 2,3,5-trifluoro-4-hydroxybenzoic acid (39.8 mg, 0.207 mmol), 1-methyl-1H-imidazole (55.0 μL, 0.690 mmol), and 2-hydroxypyridine 1-oxide (26.8 mg, 0.241 mmol). After the reaction mixture had been stirred for 20 minutes at room temperature, 1-[3-(dimethylamino)propyl]-3-ethylcarbodiimide hydrochloride (98%, 33.8 mg, 0.173 mmol) was added and stirring was continued for 18 hours. The reaction mixture was then diluted with water (10 mL), acidified to pH 4 by addition of 1 M hydrochloric acid, and extracted three times with ethyl acetate. The combined organic layers were washed five times with water, dried over magnesium sulfate, filtered, concentrated in vacuo, and purified using reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 5% to 95% B over 8.54 minutes, followed by 95% B for 1.46 minutes; Flow rate: 25 mL/minute) to provide 2,3,5-trifluoro-4-hydroxy-N-{[(1r,4r)-4-{6-[4-(trifluoromethyl)-1H-pyrazol-1-yl]-2H-indazol-2-yl}cyclohexyl]methyl}benzamide, trifluoroacetate salt (P27). Yield: 41.1 mg, 63.1 μmol, 39% over 2 steps. LCMS m/z 538.5 [M+H]⁺. Retention time: 3.11 minutes (Analytical conditions. Column: Waters Atlantis dC18, 4.6×50 mm, 5 μm; Mobile phase A: water containing 0.05% trifluoroacetic acid (v/v); Mobile phase B: acetonitrile containing 0.05% trifluoroacetic acid (v/v); Gradient: 5.0% to 95% B over 4.0 minutes, then 95% B for 1.0 minute; Flow rate: 2 mL/minute).

### Preparation P28

### 3,5-Difluoro-4-hydroxy-N-{[(1r,4r)-4-{6-[1-(oxan-4-yl)-1H-pyrazol-4-yl]-2H-indazol-2-yl}cyclohexyl]methyl}benzamide (P28)

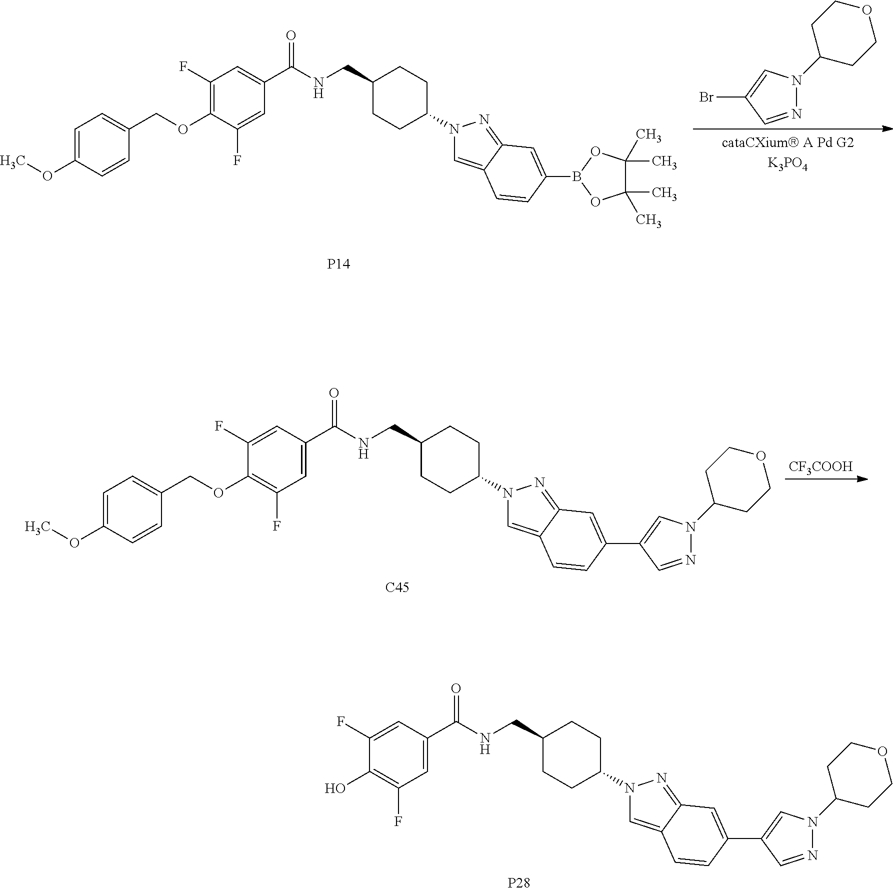

Step 1. Synthesis of 3,5-difluoro-4-[(4-methoxyphenyl)methoxy]-N-{[(1r,4r)-4-{6-[1-(oxan-4-yl)-1H-pyrazol-4-yl]-2H-indazol-2-yl}cyclohexyl]methyl}benzamide (C45): This experiment was carried out in library format. A solution of P14 (60 mg, 100 μmol) in 1,4-dioxane (1 mL) was added to 4-bromo-1-(oxan-4-yl)-1H-pyrazole (150 μmol). Aqueous tripotassium phosphate solution (1.5 M; 0.20 mL, 300 μmol) was then added, followed by chloro[(di(1-adamantyl)-N-butylphosphine)-2-(2-aminobiphenyl)]palladium(II) (cataCXium® A Pd G2; 5 μmol), whereupon the reaction vial was capped and shaken at 100° C. for 16 hours. After removal of solvent using a Speedvac® concentrator, the residue was mixed with water (1 mL), extracted with ethyl acetate (3×1.5 mL), and concentrated again, providing C45; this material was progressed directly to the following step.

Step 2. Synthesis of 3,5-difluoro-4-hydroxy-N-{[(1r,4r)-4-{6-[1-(oxan-4-yl)-1H-pyrazol-4-yl]-2H-indazol-2-yl}cyclohexyl]methyl}benzamide (P28): This experiment was carried out in library format. A solution of trifluoroacetic acid (0.2 mL) in dichloromethane (0.8 mL) was added to C45 (from the previous step; 5100 μmol), whereupon the reaction vial was capped and shaken at 30° C. for 16 hours. After removal of solvent using a Speedvac® concentrator, reversed-phase HPLC (Column: YMC-Actus Triart C18, 30×150 mm, 5 μm; Mobile phase A: water containing 0.225% formic acid; Mobile phase B: acetonitrile; Gradient: 35% to 75% B; Flow rate: 35 mL/minute) afforded 3,5-difluoro-4-hydroxy-N-{[(1r,4r)-4-{6-[1-(oxan-4-yl)-1H-pyrazol-4-yl]-2H-indazol-2-yl}cyclohexyl]methyl}benzamide (P28). Yield: 13.7 mg, 22.2 μmol, 22% over 2 steps. LCMS m/z 536 [M+H]⁺. Retention time: 2.77 minutes (Column: Waters XBridge C18, 2.1×50 mm, 5 μm; Mobile phase A: water containing 0.0375% trifluoroacetic acid; Mobile phase B: acetonitrile containing 0.01875% trifluoroacetic acid; Gradient: 1% to 5% B over 0.6 minutes; 5% to 100% B over 3.4 minutes; Flow rate: 0.8 mL/minute).

### Preparation P29

### 3,5-Difluoro-4-hydroxy-N-{[(1r,4r)-4-{5-[5-(trifluoromethyl)pyridin-2-yl]-1,2,4-oxadiazol-3-yl}cyclohexyl]methyl}benzamide (P29)

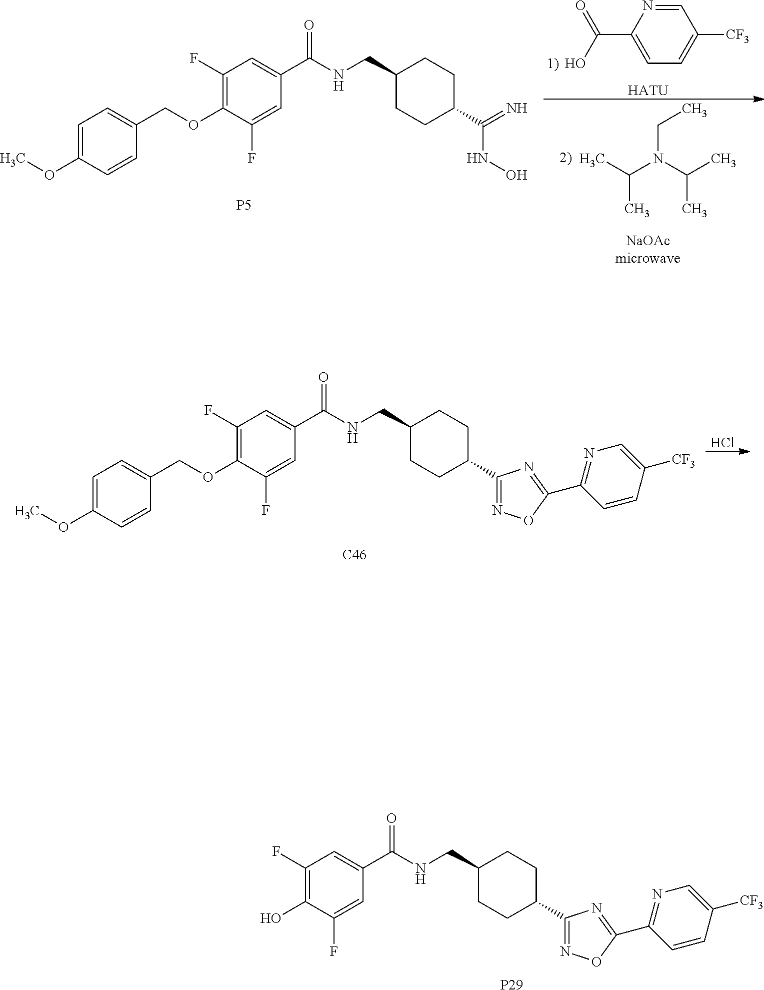

Step 1. Synthesis of 3,5-difluoro-4-[(4-methoxyphenyl)methoxy]-N-{[(1r,4r)-4-{5-[5-(trifluoromethyl) pyridin-2-yl]-1,2,4-oxadiazol-3-yl}cyclohexyl]methyl}benzamide (C46): To a 0° C. mixture of 5-(trifluoromethyl)pyridine-2-carboxylic acid (47.0 mg, 0.246 mmol), N,N-diisopropylethylamine (86.6 mg, 0.670 mmol), and O-(7-azabenzotriazol-1-yl)-N,N,N′,N′-tetramethyluronium hexafluorophosphate (HATU; 127 mg, 0.334 mmol) in dichloromethane (20 mL) was added P5 (100 mg, 0.223 mmol), whereupon the reaction mixture was stirred at room temperature for 1 hour. It was then diluted with water (20 mL) and extracted with dichloromethane (2×20 mL); the combined organic layers were washed with saturated aqueous sodium chloride solution (2×20 mL), dried over sodium sulfate, filtered, and concentrated in vacuo. Silica gel chromatography (Gradient: 0% to 5% methanol in dichloromethane) afforded the acyl intermediate as a white solid. Yield: 80 mg, 0.13 mmol, 58%. LCMS m/z 621.3 [M+H]⁺.

Sodium acetate (31.7 mg, 0.386 mmol) was added to a solution of the acyl intermediate (80 mg, 0.13 mmol) in a mixture of ethanol (4 mL) and water (1 mL). After the reaction mixture had been stirred at 100° C. for 1 hour under microwave irradiation, it was concentrated in vacuo. Silica gel chromatography (Gradient: 0% to 7% methanol in dichloromethane) provided C46 as a white solid. Yield: 40 mg, 66 μmol, 51% from the acyl intermediate. LCMS m/z 625.3 [M+Na⁺].

Step 2. Synthesis of 3,5-difluoro-4-hydroxy-N-{[(1r,4r)-4-{5-[5-(trifluoromethyl)pyridin-2-yl]-1,2,4-oxadiazol-3-yl}cyclohexyl]methyl}benzamide (P29): A solution of hydrogen chloride in 1,4-dioxane (4 M; 1 mL) was added to a solution of C46 (40 mg, 66 μmol) in dichloromethane (5 mL). The reaction mixture was stirred at room temperature for 2 hours, whereupon it was concentrated in vacuo, diluted with dichloromethane (10 mL), and treated with sodium bicarbonate (10 mg, 0.12 mmol). After removal of solvents under reduced pressure, the residue was subjected to silica gel chromatography (Gradient: 0% to 6% methanol in dichloromethane), followed by reversed-phase HPLC (Column: Waters XBridge C18, 19×100 mm, 5 μm; Mobile phase A: water containing 0.1% formic acid; Mobile phase B: acetonitrile; Gradient: 50% to 60% B; Flow rate: 20 mL/minute), to afford 3,5-difluoro-4-hydroxy-N-{[(1r,4r)-4-{5-[5-(trifluoromethyl)pyridin-2-yl]-1,2,4-oxadiazol-3-yl}cyclohexyl]methyl}benzamide (P29) as a white solid. Yield: 9.0 mg, 19 μmol, 29%. LCMS m/z 483.2 [M+H]⁺. ¹H NMR (400 MHz, methanol-d₄) δ 9.09 (br s, 1H), 8.45 (d, half of AB quartet, J=8.3 Hz, 1H), 8.40 (dd, component of ABX system, J=8.4, 2.3 Hz, 1H), 7.51-7.41 (m, 2H), 3.27 (d, J=6.9 Hz, 2H), 2.91 (tt, J=12.2, 3.4 Hz, 1H), 2.24-2.14 (m, 2H), 2.03-1.92 (m, 2H), 1.80-1.59 (m, 3H), 1.29-1.15 (m, 2H).

### Preparation P30

### N-[(4-{5-[5-(Difluoromethyl)pyrazin-2-yl]-1,2,4-oxadiazol-3-yl}bicyclo[2.2.2]octan-1-yl)methyl]-3,5-difluoro-4-hydroxybenzamide, ammonium salt (P30)

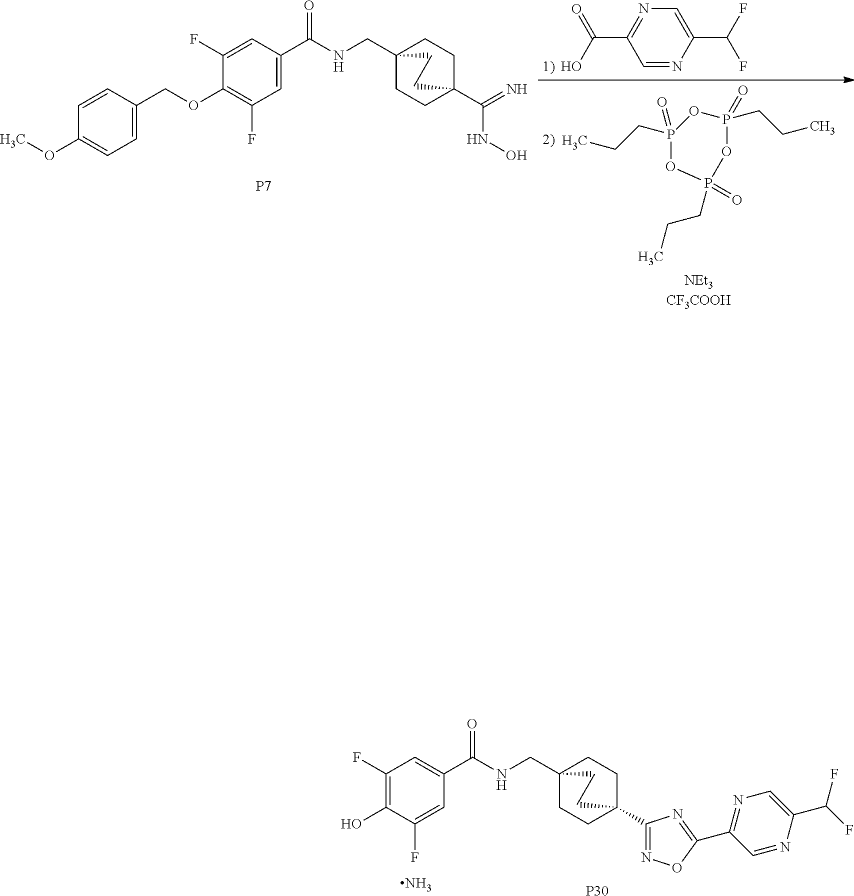

This reaction was carried out in library format.

A stock solution of P7 (300 mg, 0.634 mmol) in ethyl acetate (6 mL) was employed; 1 mL of this solution (0.106 mmol of P7) was treated with 5-(difluoromethyl)pyrazine-2-carboxylic acid (18.3 mg, 0.105 mmol), followed by triethylamine (42.2 μL, 0.303 mmol) and 2,4,6-tripropyl-1,3,5,2,4,6-trioxatriphosphinane 2,4,6-trioxide (50% solution by weight in ethyl acetate; 0.15 mL, 0.25 mmol) The reaction vial was heated at 100° C. until oxadiazole formation had occurred, whereupon it was cooled to room temperature, diluted with ethyl acetate (3 mL) and washed sequentially with water (2×3 mL) and saturated aqueous sodium chloride solution (3 mL). The organic layer was concentrated in vacuo, and the residue was dissolved in 1,1,1,3,3,3-hexafluoropropan-2-ol, treated with 1 equivalent of trifluoroacetic acid, and stirred until phenol deprotection was complete. Removal of solvent under reduced pressure was followed by reversed-phase HPLC (Column: Waters XBridge C18, 19×100 mm, 5 μm; Mobile phase A: water containing 0.03% ammonium hydroxide; Mobile phase B: acetonitrile containing 0.03% ammonium hydroxide; Gradient: 5% to 95% B; Flow rate: 25 mL/minute), affording N-[(4-{5-[5-(difluoromethyl)pyrazin-2-yl]-1,2,4-oxadiazol-3-yl}bicyclo[2.2.2]octan-1-yl)methyl]-3,5-difluoro-4-hydroxybenzamide, ammonium salt (P30). Yield: 13.8 mg, 27.1 μmol, 26%. LCMS m/z 492.4 [M+H]. Retention time: 2.54 minutes (Analytical conditions. Column: Waters Atlantis dC18, 4.6×50 mm, 5 μm; Mobile phase A: water containing 0.05% trifluoroacetic acid (v/v); Mobile phase B: acetonitrile containing 0.05% trifluoroacetic acid (v/v); Gradient: 5.0% to 95% B over 4.0 minutes, then 95% B for 1.0 minute; Flow rate: 2 mL/minute).

### Preparation P31

### 2,3,5-Trifluoro-4-hydroxy-N-({4-[3-(6-methoxypyridazin-3-yl)-1,2,4-oxadiazol-5-yl]bicyclo[2.2.2]octan-1-yl}methyl)benzamide (15)

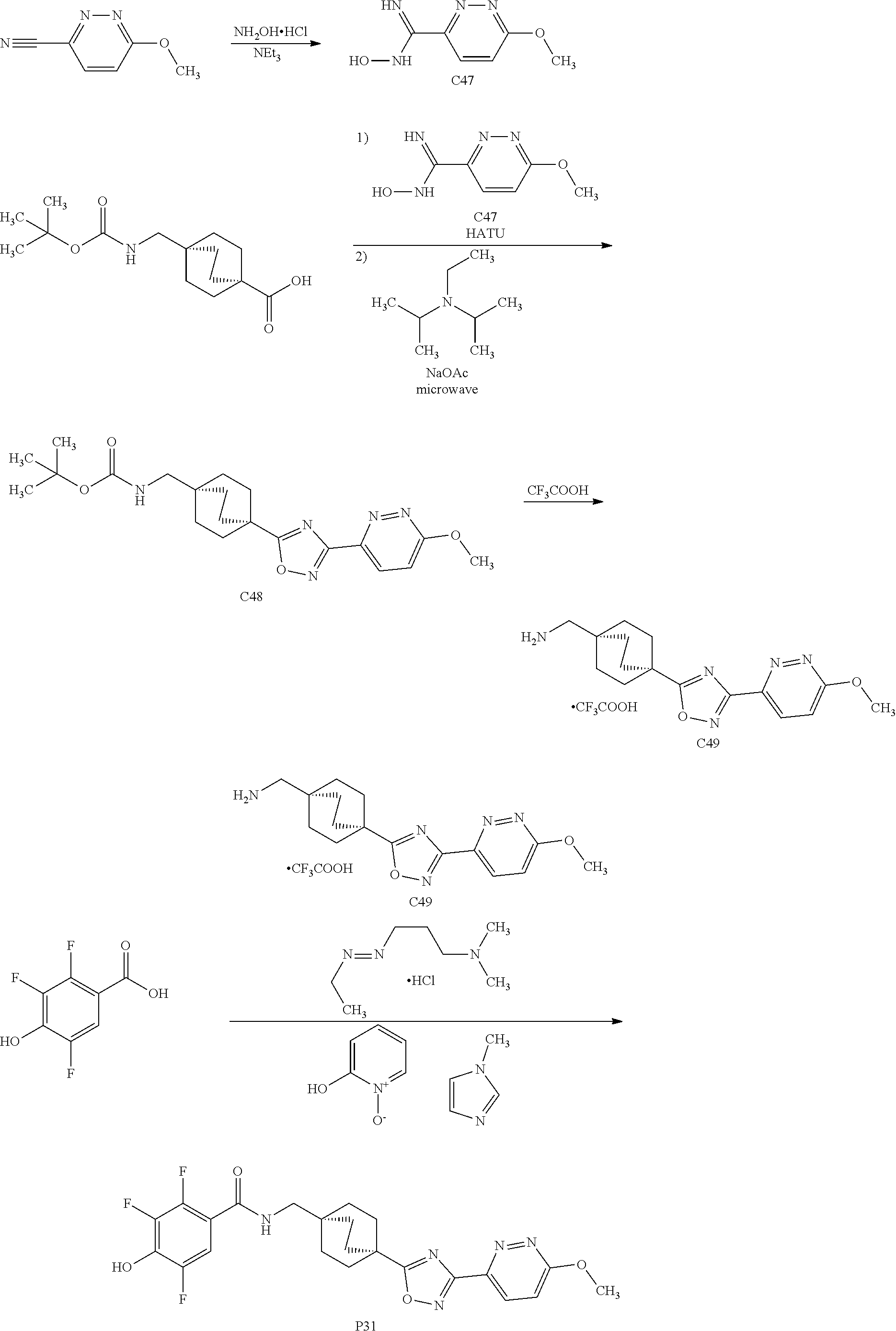

Step 1. Synthesis of N-hydroxy-6-methoxypyridazine-3-carboximidamide (C47): To a solution of 6-methoxypyridazine-3-carbonitrile (745 mg, 5.51 mmol) in methanol (3.7 mL) was added hydroxylamine hydrochloride (383 mg, 5.51 mmol), followed by triethylamine (0.776 mL, 5.57 mmol). The reaction mixture was stirred at room temperature for 4 days, whereupon it was cooled in an ice bath for 15 minutes; the precipitated solid was collected via filtration to provide C47 as a purple solid. Yield: 690 mg, 4.10 mmol, 74%. LCMS m/z 169.1 [M+H]⁺. ¹H NMR (400 MHz, DMSO-d₆) δ 10.13 (s, 1H), 7.94 (d, J=9.3 Hz, 1H), 7.22 (d, J=9.3 Hz, 1H), 5.98 (br s, 2H), 4.05 (s, 3H).

Step 2. Synthesis of tert-butyl ({4-[3-(6-methoxypyridazin-3-yl)-1,2,4-oxadiazol-5-yl]bicyclo[2.2.2]octan-1-yl}methyl)carbamate (C48): O-(7-Azabenzotriazol-1-yl)-N,N,N′,N′-tetramethyluronium hexafluorophosphate (HATU; 312 mg, 0.821 mmol) was added to a solution of C47 (155 mg, 0.547 mmol) in N,N-dimethylformamide (3 mL). After the reaction mixture had been stirred for 20 minutes, 4-{[(tert-butoxycarbonyl)amino]methyl}bicyclo[2.2.2]octane-1-carboxylic acid (101 mg, 0.601 mmol) and N,N-diisopropylethylamine (0.286 mL, 1.64 mmol) were added, and stirring was continued at room temperature for 18 hours. The reaction mixture was then diluted with water; the solid was collected via filtration and washed with water, affording the acyl intermediate as a white solid. Yield: 134 mg, 0.309 mmol, 56%. LCMS m/z 434.4 [M+H]⁺. ¹H NMR (400 MHz, methanol-d₄) δ 8.22 (d, J=9.3 Hz, 1H), 7.21 (d, J=9.3 Hz, 1H), 6.61-6.52 (m, 1H; assumed to be the amide proton, slow to exchange), 4.14 (s, 3H), 2.83 (d, J=6.5 Hz, 2H), 2.00-1.91 (m, 6H), 1.54-1.45 (m, 6H), 1.44 (s, 9H).

The acyl intermediate (134 mg, 0.309 mmol) and sodium acetate (51.2 mg, 0.624 mmol) were taken up in a mixture of water (0.1 mL) and ethanol (1 mL), and the reaction vial was heated at 120° C. for 2.5 hours under microwave irradiation. The reaction mixture was then diluted with water (approximately 0.5 mL) and filtered; the filter cake was washed with ethanol to afford C48 as an off-white solid. Yield: 75 mg, 0.18 mmol, 58% from the acyl intermediate. LCMS m/z 416.4 [M+H]⁺. ¹H NMR (400 MHz, methanol-d₄) δ 8.20 (d, J=9.2 Hz, 1H), 7.33 (d, J=9.2 Hz, 1H), 6.68-6.58 (m, 1H), 4.19 (s, 3H), 2.88 (d, J=6.4 Hz, 2H), 2.13-2.02 (m, 6H), 1.63-1.53 (m, 6H), 1.45 (s, 9H).

Step 3. Synthesis of 1-{4-[3-(6-methoxypyridazin-3-yl)-1,2,4-oxadiazol-5-yl]bicyclo[2.2.2]octan-1-yl}methanamine, trifluoroacetate salt (C49): Trifluoroacetic acid (0.15 mL, 1.9 mmol) was added drop-wise to a 0° C. solution of C48 (75 mg, 0.18 mmol) in dichloromethane (2 mL). After the reaction mixture had been stirred for 30 minutes, trifluoroacetic acid (0.15 mL, 1.9 mmol) was again added; 30 minutes later, the reaction mixture was treated once more with trifluoroacetic acid (20 μL, 0.26 mmol) and stirred for an additional 5 minutes. It was then concentrated in vacuo, and the residue was azeotroped once with toluene and once with dichloromethane, affording C49 as an oil (84 mg). Most of this material was used in the following step. LCMS m/z 316.2 [M+H]⁺.

Step 4. Synthesis of 2,3,5-trifluoro-4-hydroxy-N-({4-[3-(6-methoxypyridazin-3-yl)-1,2,4-oxadiazol-5-yl]bicyclo[2.2.2]octan-1-yl}methyl)benzamide (P31): A solution of C49 (from the previous step; 84 mg, 50.18 mmol) in a mixture of N,N-dimethylformamide (1.8 mL) and water (0.41 mL) was treated sequentially with 2,3,5-trifluoro-4-hydroxybenzoic acid (41.9 mg, 0.218 mmol), 1-methyl-1H-imidazole (43.4 μL, 0.544 mmol), and 2-hydroxypyridine 1-oxide (20.2 mg, 0.182 mmol). After the reaction mixture had been stirred for 20 minutes at room temperature, 1-[3-(dimethylamino)propyl]-3-ethylcarbodiimide hydrochloride (35.5 mg, 0.185 mmol) was added; stirring was continued at room temperature for 18 hours, whereupon the reaction mixture was diluted with water (10 mL), acidified to pH 4 by addition of methanesulfonic acid, and extracted 3 times with ethyl acetate. The combined organic layers were washed 5 times with water, dried over magnesium sulfate, filtered, and concentrated in vacuo. Purification via reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 5% to 95% B over 8.54 minutes, followed by 95% B for 1.46 minutes; Flow rate: 25 mL/minute) provided 2,3,5-trifluoro-4-hydroxy-N-({4-[3-(6-methoxypyridazin-3-yl)-1,2,4-oxadiazol-5-yl]bicyclo[2.2.2]octan-1-yl}methyl)benzamide (P31). Yield: 26.6 mg, 54.3 μmol, 30% over 2 steps. LCMS m/z 490.4 [M+H]⁺. Retention time: 2.57 minutes (Analytical conditions. Column: Waters Atlantis dC18, 4.6×50 mm, 5 μm; Mobile phase A: water containing 0.05% trifluoroacetic acid (v/v); Mobile phase B: acetonitrile containing 0.05% trifluoroacetic acid (v/v); Gradient: 5.0% to 95% B over 4.0 minutes, then 95% B for 1.0 minute; Flow rate: 2 mL/minute).

### Preparation P32

### 3,5-Difluoro-N-{[(1r,4r)-4-(6-fluoro-2H-indazol-2-yl)cyclohexyl]methyl}-4-hydroxybenzamide, ammonium salt (P32)

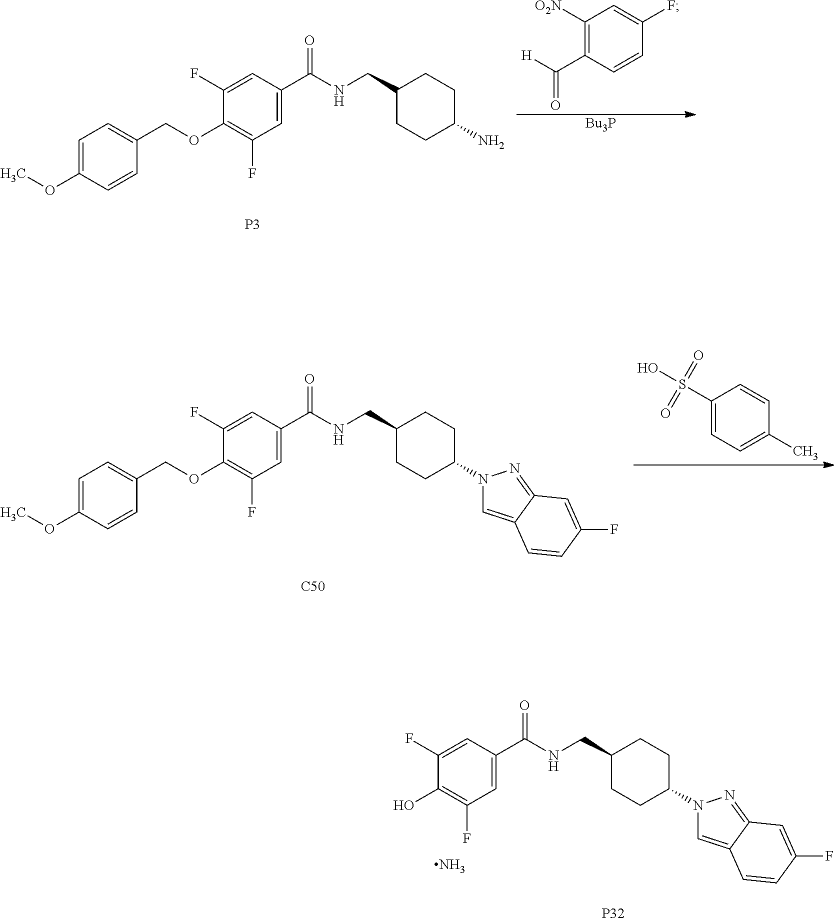

Step 1. Synthesis of 3,5-difluoro-N-{[(1r,4r)-4-(6-fluoro-2H-indazol-2-yl)cyclohexyl]methyl}-4-[(4-methoxyphenyl)methoxy]benzamide (C50): This reaction was carried out in library format. A solution of P3 (60.7 mg, 0.150 mmol) in propan-2-ol (0.6 mL) was added to 4-fluoro-2-nitrobenzaldehyde (0.15 mmol). The reaction vial was capped, then evacuated and charged with nitrogen. This evacuation cycle was repeated twice, whereupon the reaction mixture was shaken at 80° C. for 4 hours before being cooled to room temperature. After addition of tributylphosphine (0.1 mL, 0.4 mmol), the reaction mixture was shaken at 80° C. for 18 hours. It was then partitioned between half-saturated aqueous sodium bicarbonate solution (1.5 mL) and ethyl acetate (2.4 mL) and subjected to vortexing. The organic layer was eluted through a solid-phase extraction cartridge (6 mL) charged with sodium sulfate (˜1 g); this extraction procedure was repeated twice, and the combined eluents were concentrated in vacuo to provide C50, which was taken directly to the following step.

Step 2. Synthesis of 3,5-difluoro-N-{[(1r,4r)-4-(6-fluoro-2H-indazol-2-yl)cyclohexyl]methyl}-4-hydroxybenzamide, ammonium salt (P32): This reaction was carried out in library format. A solution of p-toluenesulfonic acid (57.1 mg, 0.300 mmol) in 1,1,1,3,3,3-hexafluoropropan-2-ol (0.6 mL) was added to C50 (from the previous step; 50.150 mmol), and the reaction mixture was shaken at room temperature for 3 days. After removal of solvent using a Genevac concentrator, purification was carried out via reversed-phase HPLC (Column: Waters XBridge C18, 19×100 mm, 5 μm; Mobile phase A: water containing 0.03% ammonium hydroxide; Mobile phase B: acetonitrile containing 0.03% ammonium hydroxide; Gradient: 5% to 95% B over 8.54 minutes, then 95% B for 1.46 minutes; Flow rate: 25 mL/minute) to afford 3,5-difluoro-N-{[(1r,4r)-4-(6-fluoro-2H-indazol-2-yl)cyclohexyl]methyl}-4-hydroxybenzamide, ammonium salt (P32). Yield: 11.4 mg, 27.1 μmol, 18% over 2 steps. LCMS m/z 404.4 [M+H]⁺. Retention time: 2.61 minutes (Analytical conditions. Column: Waters Atlantis dC18, 4.6×50 mm, 5 μm; Mobile phase A: water containing 0.05% trifluoroacetic acid (v/v); Mobile phase B: acetonitrile containing 0.05% trifluoroacetic acid (v/v); Gradient: 5.0% to 95% B over 4.0 minutes, then 95% B for 1.0 minute; Flow rate: 2 mL/minute).

### Preparation P33

### N-{[(1r,4r)-4-{3-[6-(2,2-Dimethylpropanamido)pyridazin-3-yl]-1,2,4-oxadiazol-5-yl}cyclohexyl]methyl}-3,5-difluoro-4-hydroxybenzamide, ammonium salt (P33)

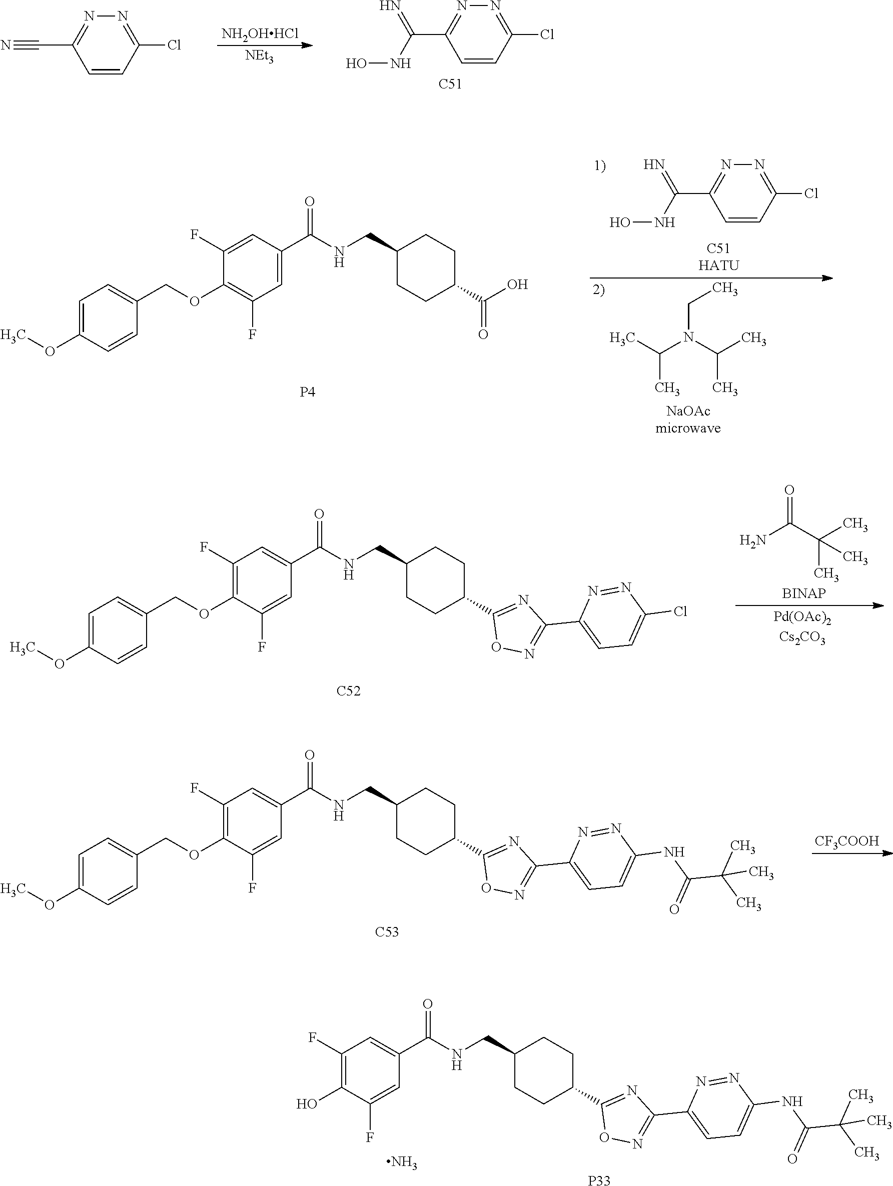

Step 1. Synthesis of 6-chloro-N-hydroxypyridazine-3-carboximidamide (C51): To a solution of 6-chloropyridazine-3-carbonitrile (698 mg, 5.00 mmol) in methanol (15 mL) was added hydroxylamine hydrochloride (382 mg, 5.50 mmol), followed by triethylamine (0.775 mL, 5.56 mmol). The reaction mixture was stirred for 2 hours, whereupon the solids were collected via filtration, providing C51 as a brown solid. Yield: 465 mg, 2.69 mmol, 54%. LCMS m/z 173.1 [M+H]⁺. ¹H NMR (400 MHz, DMSO-d₆) δ 10.43 (s, 1H), 8.08 (d, J=9.1 Hz, 1H), 7.88 (d, J=9.0 Hz, 1H), 6.15 (br s, 2H).

Step 2. Synthesis of N-({(1r,4r)-4-[3-(6-chloropyridazin-3-yl)-1,2,4-oxadiazol-5-yl]cyclohexyl}methyl)-3,5-difluoro-4-[(4-methoxyphenyl)methoxy]benzamide (C52): A solution of P4 (595 mg, 1.37 mmol) in N,N-dimethylformamide (9 mL) was treated with O-(7-azabenzotriazol-1-yl)-N,N,N′N′-tetramethyluronium hexafluorophosphate (HATU; 783 mg, 2.06 mmol). After 30 minutes, C51 (261 mg, 1.51 mmol) and N,N-diisopropylethylamine (0.717 mL, 4.12 mmol) were added, whereupon the reaction mixture was stirred for 18 hours at room temperature. The precipitate was collected by filtration and washed with dichloromethane to afford the acyl intermediate as an off-white solid. Yield: 358 mg, 0.609 mmol, 44%. LCMS m/z 588.3 [M+H]⁺. ¹H NMR (400 MHz, DMSO-d₆) δ 8.52 (br t, J=5.8 Hz, 1H), 8.15 (d, J=9.0 Hz, 1H), 8.01 (d, J=9.0 Hz, 1H), 7.64-7.55 (m, 2H), 7.34 (d, J=8.6 Hz, 2H), 7.29 (br s, 2H), 6.92 (d, J=8.6 Hz, 2H), 5.17 (s, 2H), 3.74 (s, 3H), 3.12 (dd, J=6, 6 Hz, 2H), 2.57-2.44 (m, 1H, assumed; almost entirely obscured by solvent peak), 2.05-1.96 (m, 2H), 1.85-1.75 (m, 2H), 1.61-1.48 (m, 1H), 1.47-1.32 (m, 2H), 1.06-0.92 (m, 2H).

A portion of the acyl intermediate (219 mg, 0.372 mmol) and sodium acetate (61.7 mg, 0.752 mmol), in a mixture of ethanol (4.5 mL) and water (0.45 mL), was heated at 120° C. for 1 hour under microwave irradiation. The resulting solid was isolated via filtration and washed with a 10:1 mixture of ethanol and water, providing C52 as a white solid. Yield: 172 mg, 0.302 mmol, 81% from the acyl intermediate. LCMS m/z 570.3 (chlorine isotope pattern observed) [M+H]⁺. ¹H NMR (400 MHz, DMSO-d₆) δ 8.55 (br t, J=5.8 Hz, 1H), 8.32 (d, J=9.0 Hz, 1H), 8.14 (d, J=8.9 Hz, 1H), 7.65-7.56 (m, 2H), 7.34 (d, J=8.6 Hz, 2H), 6.92 (d, J=8.6 Hz, 2H), 5.17 (s, 2H), 3.74 (s, 3H), 3.20-3.09 (m, 3H), 2.25-2.14 (m, 2H), 1.92-1.83 (m, 2H), 1.69-1.51 (m, 3H), 1.23-1.08 (m, 2H).

Step 3. Synthesis of N-{[(1r,4r)-4-{3-[6-(2,2-dimethylpropanamido)pyridazin-3-yl]-1,2,4-oxadiazol-5-yl}cyclohexyl]methyl}-3,5-difluoro-4-[(4-methoxyphenyl)methoxy]benzamide (C53): A mixture of C52 (46 mg, 81 μmol), 2,2-dimethylpropanamide (9.8 mg, 97 μmol), palladium(II) acetate (0.906 mg, 4.04 μmol), ([1,1′-binaphthalene]-2,2′-diyl)bis(diphenylphosphane) (BINAP; 5.03 mg, 8.08 μmol), and cesium carbonate (65.7 mg, 0.202 mmol) in 1,4-dioxane (1 mL) was degassed under vacuum and charged with nitrogen. This evacuation cycle was repeated twice, whereupon the reaction vial was heated at 100° C. for 18 hours. After the reaction mixture had been partitioned between water and ethyl acetate, the aqueous layer was extracted twice with ethyl acetate, and the combined organic layers were dried over magnesium sulfate, filtered, and concentrated in vacuo to provide C53 as a brown oil (64 mg). This material was taken directly to the following step. LCMS m/z 635.4 [M+H]⁺.

Step 4. Synthesis of N-{[(1r,4r)-4-{3-[6-(2,2-dimethylpropanamido)pyridazin-3-yl]-1,2,4-oxadiazol-5-yl}cyclohexyl]methyl}-3,5-difluoro-4-hydroxybenzamide, ammonium salt (P33): Trifluoroacetic acid (0.3 mL, 4 mmol) was added to a solution of C53 (from the previous step; 64 mg, 581 μmol) in dichloromethane (1 mL). The reaction mixture was stirred at room temperature for 1 hour, whereupon it was concentrated in vacuo and azeotroped twice with dichloromethane. Reversed-phase HPLC (Column: Waters XBridge C18, 19×100 mm, 5 μm; Mobile phase A: water containing 0.03% ammonium hydroxide; Mobile phase B: acetonitrile containing 0.03% ammonium hydroxide; Gradient: 5% to 50% B over 8.5 minutes, then 50% to 95% B over 0.5 minutes, then 95% B for 1.0 minute; Flow rate: 25 mL/minute) afforded N-{[(1r,4r)-4-{3-[6-(2,2-dimethylpropanamido)pyridazin-3-yl]-1,2,4-oxadiazol-5-yl}cyclohexyl]methyl}-3,5-difluoro-4-hydroxybenzamide, ammonium salt (P33). Yield: 4.2 mg, 7.9 μmol, 10% over 2 steps. LCMS m/z 515.3 [M+H]⁺. Retention time: 2.83 minutes (Analytical conditions. Column: Waters Atlantis dC18, 4.6×50 mm, 5 μm; Mobile phase A: water containing 0.05% trifluoroacetic acid (v/v); Mobile phase B: acetonitrile containing 0.05% trifluoroacetic acid (v/v); Gradient: 5.0% to 95% B over 4.0 minutes, then 95% B for 1.0 minute; Flow rate: 2 mL/minute).

### Preparation P34

### 3,5-Difluoro-4-hydroxy-N-({(1r,4r)-4-[4-(quinoxalin-6-yl)-1H-1,2,3-triazol-1-yl]cyclohexyl}methyl)benzamide (P34)

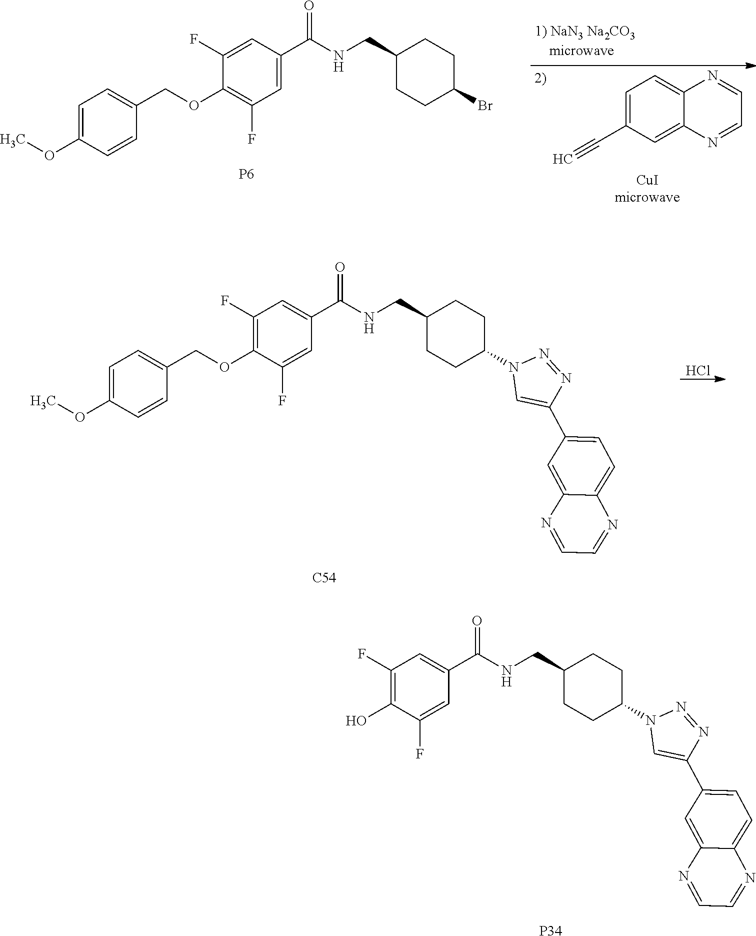

Step 1. Synthesis of 3,5-difluoro-4-[(4-methoxyphenyl)methoxy]-N-({(1r,4r)-4-[4-(quinoxalin-6-yl)-1H-1,2,3-triazol-1-yl]cyclohexyl}methyl)benzamide (C54): This reaction was carried out in library format. A solution of P6 (100 μmol) in N,N-dimethylformamide (0.50 mL) was treated with a solution of sodium azide in water (2.0 M; 0.20 mL, 400 μmol), followed by a solution of sodium carbonate in water (0.2 M; 0.10 mL, 20 μmol). The reaction vial was capped, and the reaction mixture was heated at 125° C. for 10 minutes under microwave irradiation. After the reaction mixture had cooled to room temperature, 6-ethynylquinoxaline (100 μmol) and copper(I) iodide (2.0 mg, 10 μmol) were added, and microwave irradiation was continued for 40 minutes at 125° C. When the reaction mixture had returned to room temperature, it was treated with an aqueous solution of sodium hypochlorite (8% to 10%; 1.0 mL) and the vial was shaken at 30° C. for 5 minutes; solvents were removed using a Speedvac® concentrator to provide C54. This material was progressed directly to the following step.

Step 2. Synthesis of 3,5-difluoro-4-hydroxy-N-({(1r,4r)-4-[4-(quinoxalin-6-yl)-1H-1,2,3-triazol-1-yl]cyclohexyl}methyl)benzamide (P34): This reaction was carried out in library format. To a solution of C54 (from the previous step; 5100 μmol) in dichloromethane (0.8 mL) was added a solution of hydrogen chloride in 1,4-dioxane (4 M; 0.2 mL, 800 μmol), whereupon the reaction vial was capped and shaken at 30° C. for 16 hours. After solvents had been removed using a Speedvac® concentrator, the residue was purified via reversed-phase HPLC (Column: YMC-Actus Triart C18, 30×150 mm, 5 μm; Mobile phase A: water containing ammonium hydroxide (pH 10); Mobile phase B: acetonitrile; Gradient: 10% to 50% B; Flow rate: 35 mL/minute) to afford 3,5-difluoro-4-hydroxy-N-({(1r,4r)-4-[4-(quinoxalin-6-yl)-1H-1,2,3-triazol-1-yl]cyclohexyl}methyl)benzamide (P34). Yield: 9.1 mg, 20 μmol, 20%. LCMS m/z 465 [M+H]⁺. Retention time: 2.46 minutes (Analytical conditions. Column: Waters XBridge C18, 2.1×50 mm, 5 μm; Mobile phase A: water containing 0.0375% trifluoroacetic acid; Mobile phase B: acetonitrile containing 0.01875% trifluoroacetic acid; Gradient: 1% to 5% B over 0.6 minutes; 5% to 100% B over 3.4 minutes; Flow rate: 0.8 mL/minute).

### Preparation P35

### 2,3,5-Trifluoro-4-hydroxy-N-({(1r,4r)-4-[6-(4-methylpiperazin-1-yl)-2H-indazol-2-yl]cyclohexyl}methyl)benzamide, trifluoroacetate salt (P35)

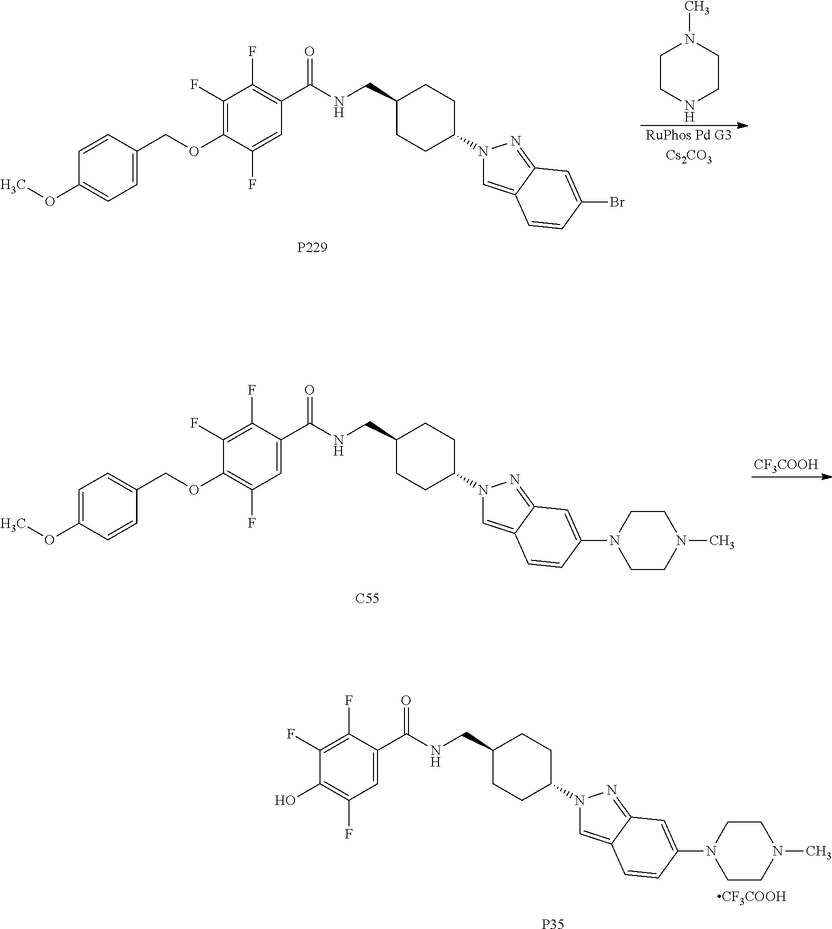

Step 1. Synthesis of 2,3,5-trifluoro-4-[(4-methoxyphenyl)methoxy]-N-({(1r,4r)-4-[6-(4-methylpiperazin-1-yl)-2H-indazol-2-yl]cyclohexyl}methyl)benzamide (C55): In a glove box under nitrogen, a scintillation vial was charged with P229 (100 mg, 0.166 mmol), cesium carbonate (162 mg, 0.497 mmol), and (2-dicyclohexylphosphino-2′,6′-diisopropoxy-1,1′-biphenyl)[2-(2′-amino-1,1′-biphenyl)]palladium(II) methanesulfonate (RuPhos Pd G3; 13.9 mg, 16.6 μmol). The contents of the vial were stirred for 2 minutes, whereupon toluene (1.7 mL) was added; to the resulting solution was added 1-methylpiperazine (27.6 μL, 0.249 mmol), and the vial was transferred to a heating block. After the reaction mixture had been slowly heated to 90° C. under vigorous stirring, it was held at 90° C. overnight. It was then allowed to cool to room temperature, concentrated in vacuo, taken up in ethyl acetate (50 mL) and washed sequentially with water (3×50 mL) and saturated aqueous sodium chloride solution (25 mL). The organic layer was concentrated under reduced pressure to afford an oil (106 mg). LCMS analysis indicated that both C55 and P35 were present in this material, the bulk of which was taken directly to the following step. LCMS m/z 622.5 and 502.4 [M+H]⁺.

Step 2. Synthesis of 2,3,5-trifluoro-4-hydroxy-N-({(1r,4r)-4-[6-(4-methylpiperazin-1-yl)-2H-indazol-2-yl]cyclohexyl}methyl)benzamide, trifluoroacetate salt (P35): Trifluoroacetic acid (50 μL, 0.65 mmol) was added to a solution of C55 and P35 (from the previous step; 103 mg, 50.161 mmol) in 1,1,1,3,3,3-hexafluoropropan-2-ol (1.5 mL). After the reaction mixture had been stirred overnight at room temperature, it was concentrated in vacuo and purified via reversed-phase HPLC [Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 5% to 35% B over 8.5 minutes, then 35% to 95% B over 0.5 minutes; Flow rate: 25 mL/minute], affording 2,3,5-trifluoro-4-hydroxy-N-({(1r,4r)-4-[6-(4-methylpiperazin-1-yl)-2H-indazol-2-yl]cyclohexyl}methyl)benzamide, trifluoroacetate salt (P35). Yield: 40 mg, 65 μmol, 40% over 2 steps. LCMS m/z 502.3 [M+H]⁺. Retention time: 1.91 minutes (Analytical conditions. Column: Waters Atlantis dC18, 4.6×50 mm, 5 μm; Mobile phase A: water containing 0.05% trifluoroacetic acid (v/v); Mobile phase B: acetonitrile containing 0.05% trifluoroacetic acid (v/v); Gradient: 5.0% to 95% B over 4.0 minutes, then 95% B for 1.0 minute; Flow rate: 2 mL/minute).

### Preparation P36

### 3,5-Difluoro-4-hydroxy-N-({(1r,4r)-4-[5-(1-methyl-1H-pyrazol-3-yl)-1-oxo-1,3-dihydro-2H-isoindol-2-yl]cyclohexyl}methyl)benzamide (P36)

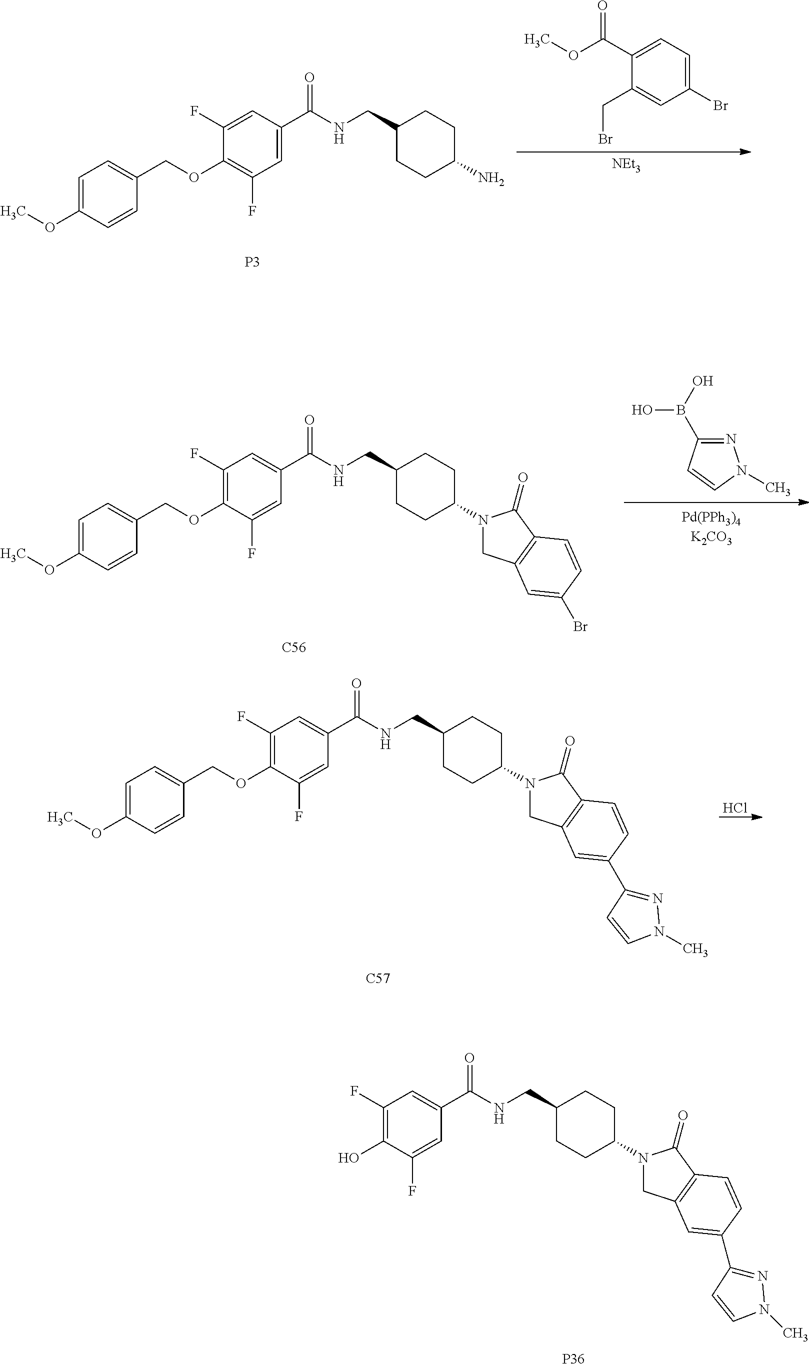

Step 1. Synthesis of N-{[(1r,4r)-4-(5-bromo-1-oxo-1,3-dihydro-2H-isoindol-2-yl)cyclohexyl]methyl}-3,5-difluoro-4-[(4-methoxyphenyl)methoxy]benzamide (C56): To a solution of P3 (400 mg, 0.989 mmol) and triethylamine (150 mg, 1.48 mmol) in toluene (10 mL) was added methyl 4-bromo-2-(bromomethyl)benzoate (305 mg, 0.990 mmol). After the reaction mixture had been stirred at 100° C. for 16 hours, it was concentrated in vacuo; silica gel chromatography (Eluent: 5% methanol in dichloromethane) afforded C56 as a white solid. Yield: 332 mg, 0.554 mmol, 56%. LCMS m/z 599.0 (bromine isotope pattern observed) [M+H]⁺. ¹H NMR (400 MHz, DMSO-d₆) δ 8.55 (br t, J=5.6 Hz, 1H), 7.84 (s, 1H), 7.69-7.55 (m, 4H), 7.33 (d, J=8.5 Hz, 2H), 6.92 (d, J=8.4 Hz, 2H), 5.17 (s, 2H), 4.43 (s, 2H), 4.04-3.92 (m, 1H), 3.74 (s, 3H), 3.12 (dd, J=6, 6 Hz, 2H), 1.89-1.70 (m, 4H), 1.63-1.46 (m, 3H), 1.19-1.04 (m, 2H).

Step 2. Synthesis of 3,5-difluoro-4-[(4-methoxyphenyl)methoxy]-N-({(1r,4r)-4-[5-(1-methyl-1H-pyrazol-3-yl)-1-oxo-1,3-dihydro-2H-isoindol-2-yl]cyclohexyl}methyl)benzamide (C57): To a mixture of C56 (100 mg, 0.167 mmol), (1-methyl-1H-pyrazol-3-yl)boronic acid (25.2 mg, 0.200 mmol), and potassium carbonate (69.2 mg, 0.501 mmol) in 1,4-dioxane (10 mL) was added tetrakis(triphenylphosphine)palladium(0) (19.3 mg, 16.7 μmol), whereupon the reaction mixture was stirred at 100° C. for 16 hours. After solvents had been removed via concentration in vacuo, silica gel chromatography (Eluent: 5% methanol in dichloromethane) provided C57 as an oil. Yield: 42 mg, 70 μmol, 42%. LCMS m/z 601.2 [M+H]⁺.

Step 3. Synthesis of 3,5-difluoro-4-hydroxy-N-({(1r,4r)-4-[5-(1-methyl-1H-pyrazol-3-yl)-1-oxo-1,3-dihydro-2H-isoindol-2-yl]cyclohexyl}methyl)benzamide (P36): To a solution of C57 (37 mg, 62 μmol) in dichloromethane (5 mL) was added a solution of hydrogen chloride in 1,4-dioxane (4 M; 1 mL). The reaction mixture was stirred at 25° C. for 1 hour, whereupon it was concentrated in vacuo; purification via reversed-phase HPLC (Column: Waters XBridge C18, 19×100 mm, 5 μm; Mobile phase A: water containing 0.1% formic acid; Mobile phase B: acetonitrile; Gradient: 25% to 45% B; Flow rate: 20 mL/minute), provided 3,5-difluoro-4-hydroxy-N-({(1r,4r)-4-[5-(1-methyl-1H-pyrazol-3-yl)-1-oxo-1,3-dihydro-2H-isoindol-2-yl]cyclohexyl}methyl)benzamide (P36). Yield: 15.8 mg, 32.9 μmol, 53%. LCMS m/z 481.2 [M+H]⁺. ¹H NMR (400 MHz, DMSO-d₆) δ 8.37 (br t, J=5.8 Hz, 1H), 7.97 (br s, 1H), 7.89 (dd, J=7.9, 1.4 Hz, 1H), 7.77 (d, J=2.3 Hz, 1H), 7.66 (d, J=7.9 Hz, 1H), 7.59-7.47 (m, 2H), 6.79 (d, J=2.3 Hz, 1H), 4.46 (s, 2H), 4.00 (tt, J=12.2, 3.8 Hz, 1H), 3.90 (s, 3H), 3.12 (dd, J=6, 6 Hz, 2H), 1.90-1.73 (m, 4H), 1.65-1.49 (m, 3H), 1.20-1.05 (m, 2H).

### Preparation P37

### 2,3,5-Trifluoro-4-hydroxy-N-[(4-{5-[2-(4-methylpiperazin-1-yl)pyrimidin-4-yl]-1,2,4-oxadiazol-3-yl}bicyclo[2.2.2]octan-1-yl)methyl]benzamide, hydrochloride salt (P37)

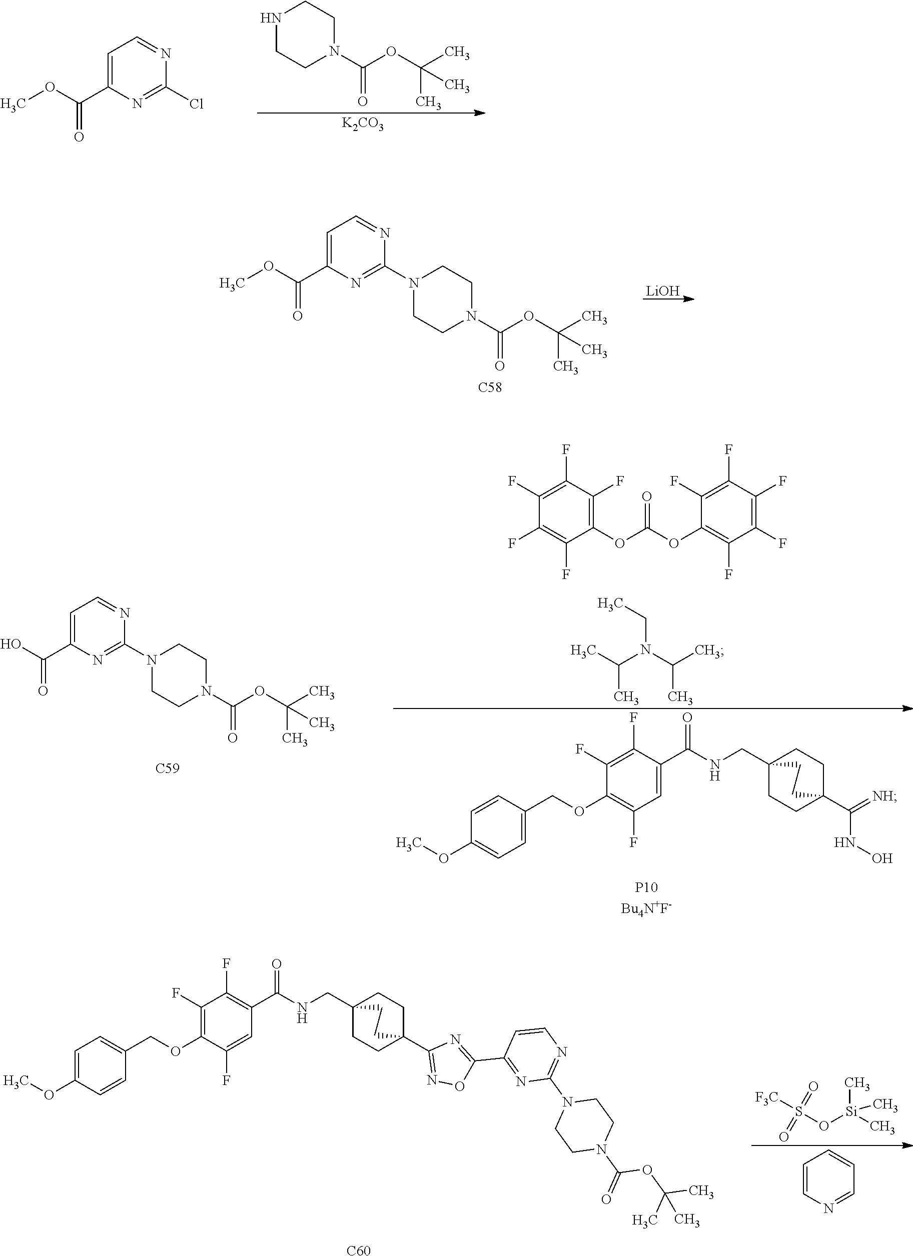

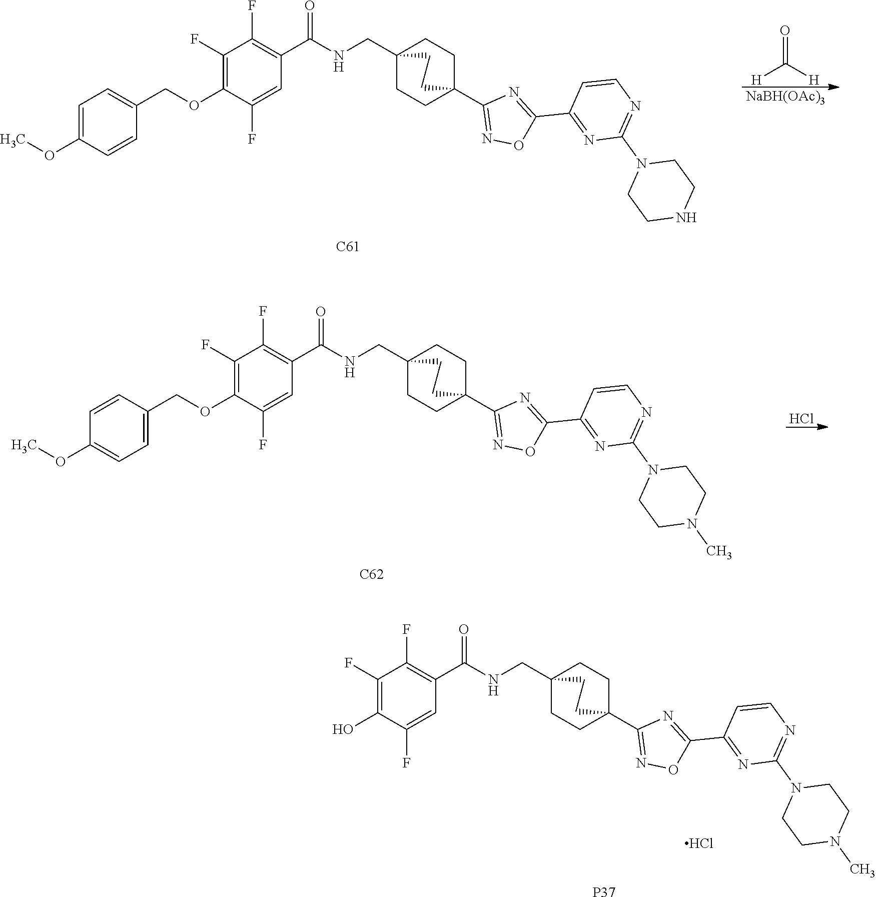

Step 1. Synthesis of methyl 2-[4-(tert-butoxycarbonyl)piperazin-1-yl]pyrimidine-4-carboxylate (C58): Potassium carbonate (2.18 g, 15.8 mmol) was added to a solution of methyl 2-chloropyrimidine-4-carboxylate (95%, 956 mg, 5.26 mmol) and tert-butyl piperazine-1-carboxylate (1.00 g, 5.37 mmol) in acetonitrile (26 mL), whereupon the reaction mixture was stirred at 65° C. After 1.5 hours, LCMS analysis indicated the presence of C58: LCMS m/z 267.2 [(M-2-methylprop-1-ene)+H]⁺. The reaction mixture was allowed to stir at 65° C. for an additional hour, and was then diluted with water and extracted three times with dichloromethane. The combined organic layers were concentrated in vacuo to afford C58 as a yellow solid (1.75 g), the bulk of which was progressed to the following step. ¹H NMR (400 MHz, chloroform-d) δ 8.51 (d, J=4.8 Hz, 1H), 7.14 (d, J=4.8 Hz, 1H), 3.96 (s, 3H), 3.92-3.84 (m, 4H), 3.55-3.47 (m, 4H), 1.49 (s, 9H).

Step 2. Synthesis of 2-[4-(tert-butoxycarbonyl)piperazin-1-yl]pyrimidine-4-carboxylic acid (C59): A solution of lithium hydroxide (1.26 g, 52.6 mmol) in a mixture of tetrahydrofuran (10 mL), water (10 mL), and methanol (5 mL) was added to C58 (from the previous step; 1.70 g, 55.11 mmol). The reaction mixture was heated at 50° C. for 1 hour, allowed to cool to room temperature, and concentrated in vacuo to remove most of the solvent. After acidification of the residue to pH 2 to 3 by addition of 1 M hydrochloric acid, the mixture was extracted three times with ethyl acetate. At this point, the aqueous layer was acidified again to bring the pH to 2, and extracted twice with ethyl acetate. All of the organic layers were combined, dried over magnesium sulfate, filtered, and concentrated under reduced pressure to provide C59 as a pale-yellow solid. Yield: 1.42 g, 4.60 mmol, 90% over 2 steps. LCMS m/z 307.2 [M−H]⁻. ¹H NMR (400 MHz, chloroform-d) δ 8.62 (d, J=4.7 Hz, 1H), 7.31 (d, J=4.7 Hz, 1H), 3.90-3.82 (m, 4H), 3.58-3.51 (m, 4H), 1.50 (s, 9H).

Step 3. Synthesis of tert-butyl 4-(4-{3-[4-({2,3,5-trifluoro-4-[(4-methoxyphenyl)methoxy]benzamido}methyl)bicyclo[2.2.2]octan-1-yl]-1,2,4-oxadiazol-5-yl}pyrimidin-2-yl)piperazine-1-carboxylate (C60): N,N-Diisopropylethylamine (0.532 mL, 3.05 mmol) was added drop-wise to a solution of C59 (345 mg, 1.12 mmol) and bis(pentafluorophenyl) carbonate (98%, 450 mg, 1.12 mmol) in tetrahydrofuran (5 mL). After the reaction mixture had been stirred at room temperature for 30 minutes, additional bis(pentafluorophenyl) carbonate (98%, 20 mg, 51 μmol) was added and stirring was continued for 10 minutes, whereupon P10 (500 mg, 1.02 mmol) was added, followed by another 30 minutes of stirring at room temperature. The reaction mixture was then treated with a solution of tetrabutylammonium fluoride in tetrahydrofuran (1.0 M; 5.09 mL, 5.09 mmol) and heated at 50° C. overnight. After cooling to room temperature, the reaction mixture was treated with a small amount of aqueous sodium bicarbonate solution, diluted with water, and extracted three times with ethyl acetate. The combined organic layers were dried over magnesium sulfate, filtered, concentrated in vacuo, and purified via chromatography on silica gel (Gradient: 30% to 100% ethyl acetate in heptane), affording C60 as a yellow solid. Yield: 474 mg (corrected for residual dichloromethane), 0.621 mmol, 61%. LCMS m/z 764.5 [M+H]⁺. ¹H NMR (400 MHz, DMSO-d₆) δ 8.67 (d, J=4.8 Hz, 1H), 8.35 (br t, J=6.3 Hz, 1H), 7.38-7.31 (m, 1H), 7.36 (br d, J=8.6 Hz, 2H), 7.30 (d, J=4.8 Hz, 1H), 6.94 (br d, J=8.7 Hz, 2H), 5.22 (s, 2H), 3.83-3.77 (m, 4H), 3.75 (s, 3H), 3.47-3.40 (m, 4H), 3.07 (d, J=6.2 Hz, 2H), 1.94-1.84 (m, 6H), 1.57-1.48 (m, 6H), 1.43 (s, 9H).

Step 4. Synthesis of 2,3,5-trifluoro-4-[(4-methoxyphenyl)methoxy]-N-[(4-{5-[2-(piperazin-1-yl)pyrimidin-4-yl]-1,2,4-oxadiazol-3-yl}bicyclo[2.2.2]octan-1-yl)methyl]benzamide (C61): A solution of C60 (840 mg, 1.10 mmol) and pyridine (0.711 mL, 8.79 mmol) in dichloromethane (36 mL) was cooled to approximately −15° C. and treated drop-wise with trimethylsilyl trifluoromethanesulfonate (0.796 mL, 4.40 mmol). The reaction mixture was stirred overnight at −15° C., although by morning the temperature of the cooling bath had reached 12° C. The reaction mixture was then cooled in an ice bath, whereupon aqueous sodium bicarbonate solution (20 mL) was slowly added and the resulting mixture was stirred for 10 minutes. The aqueous layer was adjusted to pH 10 and extracted three times with dichloromethane; the combined organic layers were washed sequentially with saturated aqueous sodium bicarbonate solution and saturated aqueous sodium chloride solution, dried over magnesium sulfate, filtered, and concentrated under reduced pressure. The residue was co-evaporated three times with dichloromethane, providing C61 as a yellow solid. Yield: 673 mg, 1.01 mmol, 92%. LCMS m/z 664.4 [M+H]⁺. ¹H NMR (400 MHz, chloroform-d) δ 8.51 (d, J=4.8 Hz, 1H), 7.59 (ddd, J=11.7, 6.8, 2.3 Hz, 1H), 7.34 (d, J=8.6 Hz, 2H), 7.22 (d, J=4.8 Hz, 1H), 6.88 (d, J=8.6 Hz, 2H), 6.62-6.50 (m, 1H), 5.24 (s, 2H), 3.96-3.86 (m, 4H), 3.80 (s, 3H), 3.30 (br d, J=6 Hz, 2H), 3.04-2.91 (m, 4H), 2.07-1.96 (m, 6H), 1.66-1.56 (m, 6H).

Step 5. Synthesis of 2,3,5-trifluoro-4-[(4-methoxyphenyl)methoxy]-N-[(4-{5-[2-(4-methylpiperazin-1-yl)pyrimidin-4-yl]-1,2,4-oxadiazol-3-yl}bicyclo[2.2.2]octan-1-yl)methyl]benzamide (C62): To a solution of C61 (100 mg, 0.151 mmol) and formaldehyde (43 mg, 1.43 mmol) in 1,2-dichloroethane (8 mL) was added sodium triacetoxyborohydride (91 mg, 0.43 mmol). After the reaction mixture had been stirred at 25° C. for 1 hour, it was subjected to an aqueous workup and extracted with dichloromethane (2×30 mL); the combined organic layers were washed with saturated aqueous sodium chloride solution, dried over sodium sulfate, filtered, and concentrated in vacuo. Silica gel chromatography (Eluent: 5% methanol in dichloromethane) provided C62 as a yellow solid. Yield: 72.0 mg, 0.106 mmol, 70%. LCMS m/z 678.2 [M+H]⁺. ¹H NMR (400 MHz, DMSO-d₆) δ 8.65 (d, J=4.9 Hz, 1H), 8.36 (br t, J=6.2 Hz, 1H), 7.37-7.30 (m, 1H), 7.36 (d, J=8.5 Hz, 2H), 7.27 (d, J=4.8 Hz, 1H), 6.94 (d, J=8.4 Hz, 2H), 5.22 (s, 2H), 3.84-3.76 (m, 4H), 3.75 (s, 3H), 3.06 (d, J=6.2 Hz, 2H), 2.45-2.34 (m, 4H), 2.23 (s, 3H), 1.92-1.84 (m, 6H), 1.58-1.47 (m, 6H).

Step 6. Synthesis of 2,3,5-trifluoro-4-hydroxy-N-[(4-{5-[2-(4-methylpiperazin-1-yl)pyrimidin-4-yl]-1,2,4-oxadiazol-3-yl}bicyclo[2.2.2]octan-1-yl)methyl]benzamide, hydrochloride salt (P37): A solution of hydrogen chloride in 1,4-dioxane (4 M: 1 mL, 4 mmol) was added to a solution of C62 (72.0 mg, 0.106 mmol) in dichloromethane (4 mL). After the reaction mixture had been stirred at 25° C. for 1 hour, it was concentrated in vacuo and purified via reversed-phase HPLC (Column: Waters XBridge C18, 19×150 mm, 5 μm, Mobile phase A: water containing 0.05% formic acid; Mobile phase B: acetonitrile; Gradient: 15% to 45% B; Flow rate: 20 mL/minute) to provide 2,3,5-trifluoro-4-hydroxy-N-[(4-{5-[2-(4-methylpiperazin-1-yl)pyrimidin-4-yl]-1,2,4-oxadiazol-3-yl}bicyclo[2.2.2]octan-1-yl)methyl]benzamide, hydrochloride salt (P37) as a white solid. Yield: 30.0 mg, 50.5 μmol, 48%. LCMS m/z 558.3 [M+H]⁺. ¹H NMR (400 MHz, DMSO-d₆), characteristic peaks: δ 8.75 (d, J=4.9 Hz, 1H), 8.19 (br t, J=6 Hz, 1H), 7.42 (d, J=4.9 Hz, 1H), 7.27 (ddd, J=11.0, 6.2, 2.4 Hz, 1H), 3.66-3.2 (m, 8H, assumed; entirely obscured by water peak), 3.07 (d, J=6.2 Hz, 2H), 2.84 (s, 3H), 1.96-1.83 (m, 6H), 1.60-1.47 (m, 6H).

Using analogous procedures, compounds of Preparations P38 to P226 were synthesized as described in Tables 1 and 2.

TABLE 1
Structure and IUPAC Name for Preparations P38-P226.

| Preparation | Structure | IUPAC Name |
|---|---|---|
| P38 | 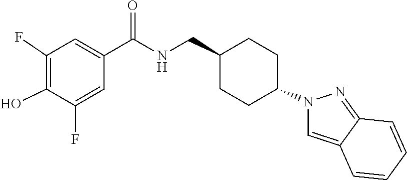 [SMILES: O=C(NC[C@H]1CC[C@H](n2cc3ccccc3n2)CC1)c1cc(F)c(O)c(F)c1] | 3,5-difluoro-4-hydroxy-N- {[(1r,4r)-4-(2H-indazol-2- yl)cyclohexyl]methyl}benzamide |
| P39 | 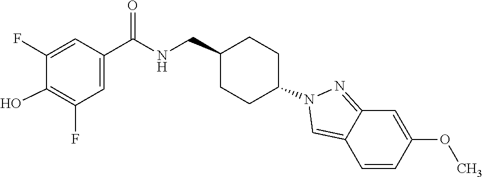 [SMILES: COc1ccc2cn([C@H]3CC[C@H](CNC(=O)c4cc(F)c(O)c(F)c4)CC3)nc2c1] | 3,5-difluoro-4-hydroxy-N- {[(1r,4r)-4-(6-methoxy-2H- indazol-2- yl)cyclohexyl]methyl}benzamide |
| P40 | 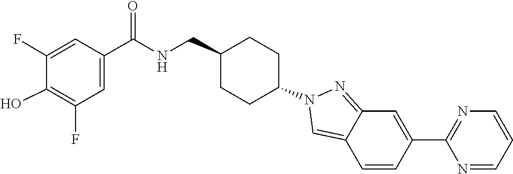 [SMILES: O=C(NC[C@H]1CC[C@H](n2cc3ccc(-c4ncccn4)cc3n2)CC1)c1cc(F)c(O)c(F)c1] | 3,5-difluoro-4-hydroxy-N- ({(1r,4r)-4-[6-(pyrimidin-2-yl)- 2H-indazol-2- yl]cyclohexyl}methyl)benzamide |
| P41 | 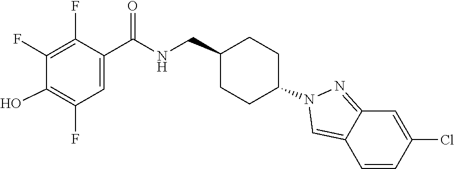 [SMILES: O=C(NC[C@H]1CC[C@H](n2cc3ccc(Cl)cc3n2)CC1)c1cc(F)c(O)c(F)c1F] | N-{[(1r,4r)-4-(6-chloro-2H- indazol-2-yl)cyclohexyl]methyl}- 2,3,5-trifluoro-4- hydroxybenzamide |
| P42 | 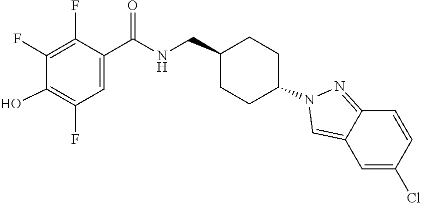 [SMILES: O=C(NC[C@H]1CC[C@H](n2cc3cc(Cl)ccc3n2)CC1)c1cc(F)c(O)c(F)c1F] | N-{[(1r,4r)-4-(5-chloro-2H- indazol-2-yl)cyclohexyl]methyl}- 2,3,5-trifluoro-4- hydroxybenzamide |
| P43 | 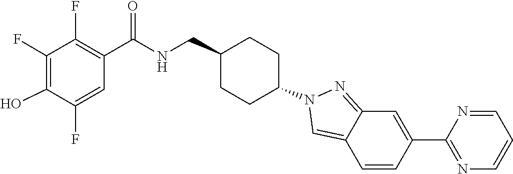 [SMILES: O=C(NC[C@H]1CC[C@H](n2cc3ccc(-c4ncccn4)cc3n2)CC1)c1cc(F)c(O)c(F)c1F] | 2,3,5-trifluoro-4-hydroxy-N- ({(1r,4r)-4-[6-(pyrimidin-2-yl)- 2H-indazol-2- yl]cyclohexyl}methyl)benzamide |
| P44 | 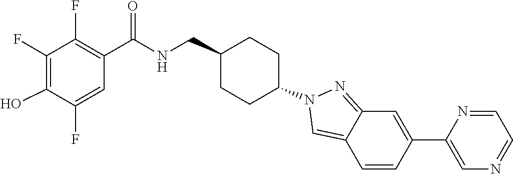 [SMILES: O=C(NC[C@H]1CC[C@H](n2cc3ccc(-c4cnccn4)cc3n2)CC1)c1cc(F)c(O)c(F)c1F] | 2,3,5-trifluoro-4-hydroxy-N- ({(1r,4r)-4-[6-(pyrazin-2-yl)-2H- indazol-2- yl]cyclohexyl}methyl)benzamide |
| P45 | 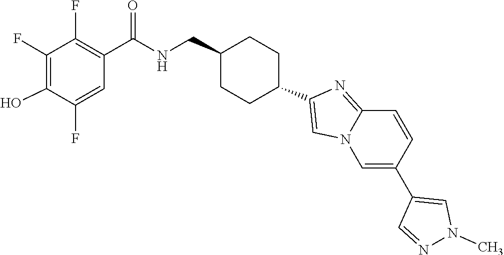 [SMILES: Cn1cc(-c2ccc3nc([C@H]4CC[C@H](CNC(=O)c5cc(F)c(O)c(F)c5F)CC4)cn3c2)cn1] | 2,3,5-trifluoro-4-hydroxy-N- ({(1r,4r)-4-[6-(1-methyl-1H- pyrazol-4-yl)imidazo[1,2- a]pyridin-2- yl]cyclohexyl}methyl)benzamide |
| P46 | 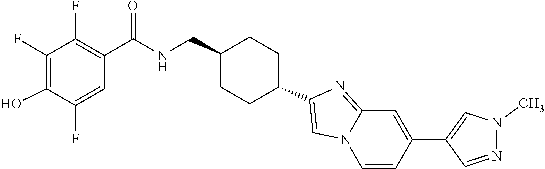 [SMILES: Cn1cc(-c2ccn3cc([C@H]4CC[C@H](CNC(=O)c5cc(F)c(O)c(F)c5F)CC4)nc3c2)cn1] | 2,3,5-trifluoro-4-hydroxy-N- ({(1r,4r)-4-[7-(1-methyl-1H- pyrazol-4-yl)imidazo[1,2- a]pyridin-2- yl]cyclohexyl}methyl)benzamide |
| P47 | 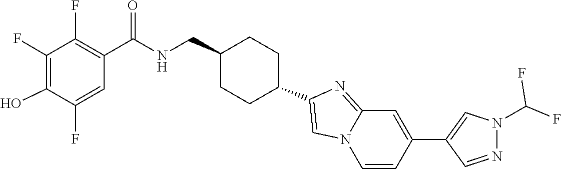 [SMILES: O=C(NC[C@H]1CC[C@H](c2cn3ccc(-c4cnn(C(F)F)c4)cc3n2)CC1)c1cc(F)c(O)c(F)c1F] | N-{[(1r,4r)-4-{7-[1- (difluoromethyl)-1H-pyrazol-4- yl]imidazo[1,2-a]pyridin-2- yl}cyclohexyl]methyl}-2,3,5- trifluoro-4- hydroxybenzamide |
| P48 | 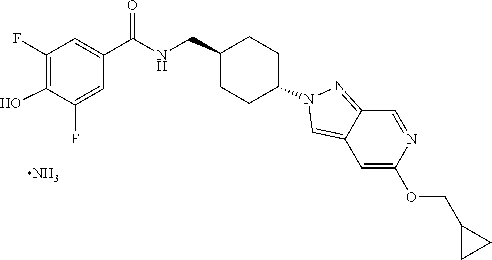 | N-({(1r,4r)-4-[5- (cyclopropylmethoxy)-2H- pyrazolo[3,4-c]pyridin-2- yl]cyclohexyl}methyl)-3,5- difluoro-4-hydroxybenzamide, ammonium salt |
| P49 | 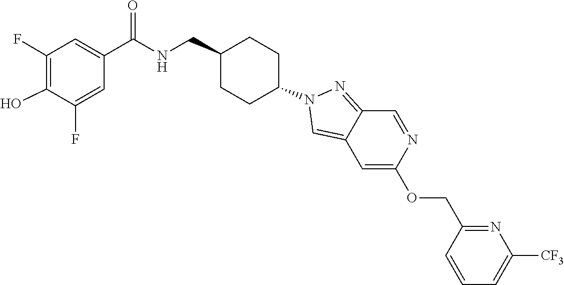 [SMILES: O=C(NC[C@H]1CC[C@H](n2cc3cc(OCc4cccc(C(F)(F)F)n4)ncc3n2)CC1)c1cc(F)c(O)c(F)c1] | 3,5-difluoro-4-hydroxy-N- {[(1r,4r)-4-(5-{[6- (trifluoromethyl)pyridin-2- yl]methoxy}-2H-pyrazolo[3,4- c]pyridin-2- yl)cyclohexyl]methyl}benzamide |
| P50 | 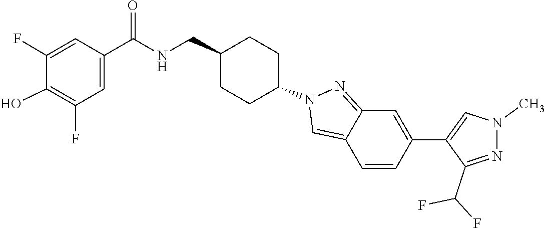 [SMILES: Cn1cc(-c2ccc3cn([C@H]4CC[C@H](CNC(=O)c5cc(F)c(O)c(F)c5)CC4)nc3c2)c(C(F)F)n1] | N-{[(1r,4r)-4-{6-[3- (difluoromethyl)-1-methyl-1H- pyrazol-4-yl]-2H-indazol-2- yl}cyclohexyl]methyl}-3,5- difluoro-4-hydroxybenzamide |
| P51 | 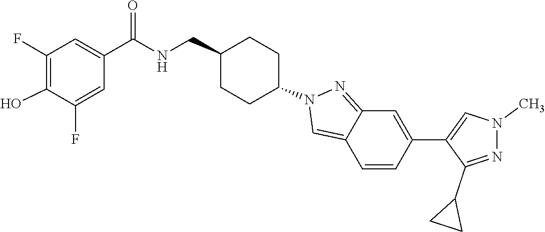 [SMILES: Cn1cc(-c2ccc3cn([C@H]4CC[C@H](CNC(=O)c5cc(F)c(O)c(F)c5)CC4)nc3c2)c(C2CC2)n1] | N-({(1r,4r)-4-[6-(3-cyclopropyl- 1-methyl-1H-pyrazol-4-yl)-2H- indazol-2-yl]cyclohexyl}methyl)- 3,5-difluoro-4- hydroxybenzamide |
| P52 | 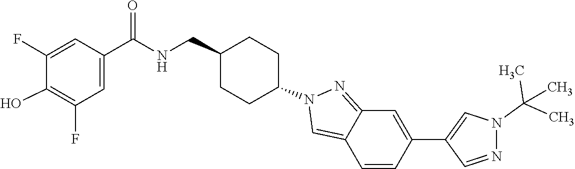 [SMILES: CC(C)(C)n1cc(-c2ccc3cn([C@H]4CC[C@H](CNC(=O)c5cc(F)c(O)c(F)c5)CC4)nc3c2)cn1] | N-({(1r,4r)-4-[6-(1-tert-butyl-1H- pyrazol-4-yl)-2H-indazol-2- yl]cyclohexyl}methyl)-3,5- difluoro-4-hydroxybenzamide |
| P53 | 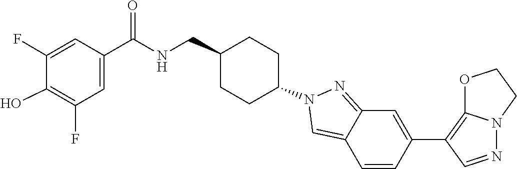 [SMILES: O=C(NC[C@H]1CC[C@H](n2cc3ccc(-c4cnn5c4OCC5)cc3n2)CC1)c1cc(F)c(O)c(F)c1] | N-({(1r,4r)-4-[6-(2,3- dihydropyrazolo[5,1- b][1,3]oxazol-7-yl)-2H-indazol- 2-yl]cyclohexyl}methyl)-3,5- difluoro-4-hydroxybenzamide |
| P54 | 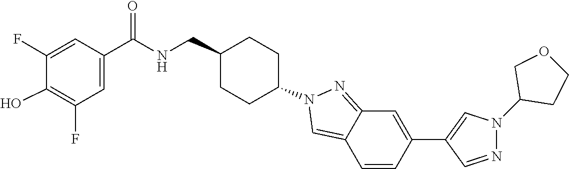 [SMILES: O=C(NC[C@H]1CC[C@H](n2cc3ccc(-c4cnn(C5CCOC5)c4)cc3n2)CC1)c1cc(F)c(O)c(F)c1] | 3,5-difluoro-4-hydroxy-N- {[(1r,4r)-4-{6-[1-(oxolan-3-yl)- 1H-pyrazol-4-yl]-2H-indazol-2- yl}cyclohexyl]methyl}benzamide |
| P55 | 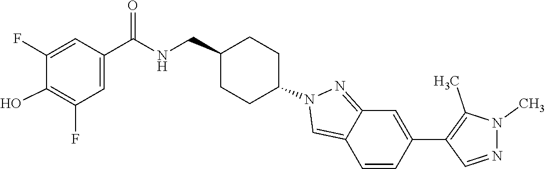 [SMILES: Cc1c(-c2ccc3cn([C@H]4CC[C@H](CNC(=O)c5cc(F)c(O)c(F)c5)CC4)nc3c2)cnn1C] | N-({(1r,4r)-4-[6-(1,5-dimethyl- 1H-pyrazol-4-yl)-2H-indazol-2- yl]cyclohexyl}methyl)-3,5- difluoro-4-hydroxybenzamide |
| P56 | 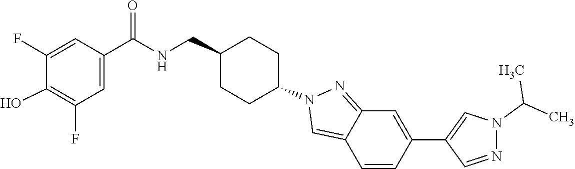 [SMILES: CC(C)n1cc(-c2ccc3cn([C@H]4CC[C@H](CNC(=O)c5cc(F)c(O)c(F)c5)CC4)nc3c2)cn1] | 3,5-difluoro-4-hydroxy-N- {[(1r,4r)-4-{6-[1-(propan-2-yl)- 1H-pyrazol-4-yl]-2H-indazol-2- yl}cyclohexyl]methyl}benzamide |
| P57 | 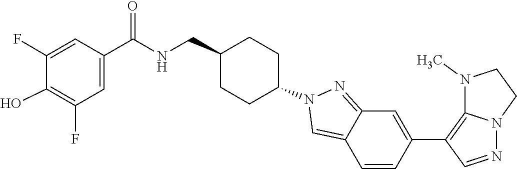 [SMILES: CN1CCn2ncc(-c3ccc4cn([C@H]5CC[C@H](CNC(=O)c6cc(F)c(O)c(F)c6)CC5)nc4c3)c21] | 3,5-difluoro-4-hydroxy-N- ({(1r,4r)-4-[6-(1-methyl-2,3- dihydro-1H-imidazo[1,2- b]pyrazol-7-yl)-2H-indazol-2- yl]cyclohexyl}methyl)benzamide |
| P58 | 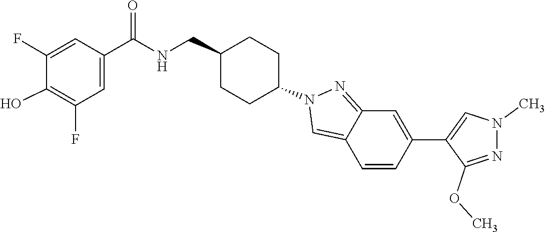 [SMILES: COc1nn(C)cc1-c1ccc2cn([C@H]3CC[C@H](CNC(=O)c4cc(F)c(O)c(F)c4)CC3)nc2c1] | 3,5-difluoro-4-hydroxy-N- ({(1r,4r)-4-[6-(3-methoxy-1- methyl-1H-pyrazol-4-yl)-2H- indazol-2- yl]cyclohexyl}methyl)benzamide |
| P59 | 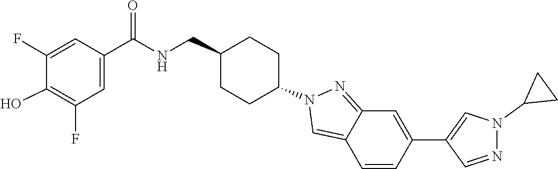 [SMILES: O=C(NC[C@H]1CC[C@H](n2cc3ccc(-c4cnn(C5CC5)c4)cc3n2)CC1)c1cc(F)c(O)c(F)c1] | N-({(1r,4r)-4-[6-(1-cyclopropyl- 1H-pyrazol-4-yl)-2H-indazol-2- yl]cyclohexyl}methyl)-3,5- difluoro-4-hydroxybenzamide |
| P60 | 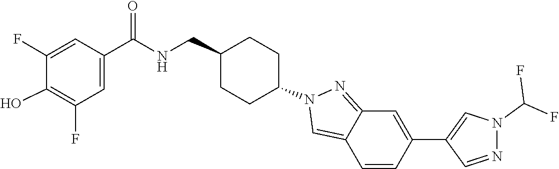 [SMILES: O=C(NC[C@H]1CC[C@H](n2cc3ccc(-c4cnn(C(F)F)c4)cc3n2)CC1)c1cc(F)c(O)c(F)c1] | N-{[(1r,4r)-4-{6-[1- (difluoromethyl)-1H-pyrazol-4- yl]-2H-indazol-2- yl}cyclohexyl]methyl}-3,5- difluoro-4-hydroxybenzamide |
| P61 | 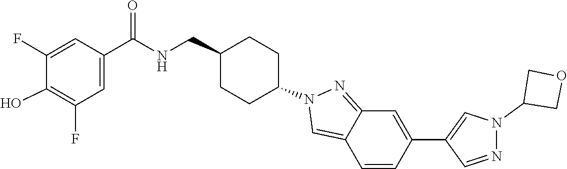 [SMILES: O=C(NC[C@H]1CC[C@H](n2cc3ccc(-c4cnn(C5COC5)c4)cc3n2)CC1)c1cc(F)c(O)c(F)c1] | 3,5-difluoro-4-hydroxy-N- {[(1r,4r)-4-{6-[1-(oxetan-3-yl)- 1H- pyrazol-4-yl]-2H-indazol-2- yl}cyclohexyl]methyl}benzamide |
| P62 | 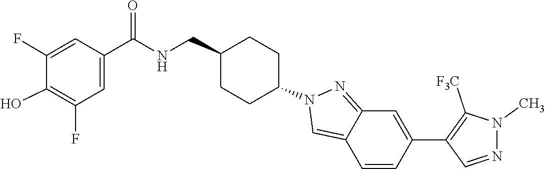 [SMILES: Cn1ncc(-c2ccc3cn([C@H]4CC[C@H](CNC(=O)c5cc(F)c(O)c(F)c5)CC4)nc3c2)c1C(F)(F)F] | 3,5-difluoro-4-hydroxy-N- {[(1r,4r)-4-{6-[1-methyl-5- (trifluoromethyl)-1H-pyrazol-4- yl]-2H-indazol-2- yl}cyclohexyl]methyl}benzamide |
| P63 | 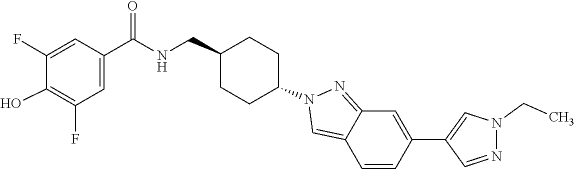 [SMILES: CCn1cc(-c2ccc3cn([C@H]4CC[C@H](CNC(=O)c5cc(F)c(O)c(F)c5)CC4)nc3c2)cn1] | N-({(1r,4r)-4-[6-(1-ethyl-1H- pyrazol-4-yl)-2H-indazol-2- yl]cyclohexyl}methyl)-3,5- difluoro-4-hydroxybenzamide |
| P64 | 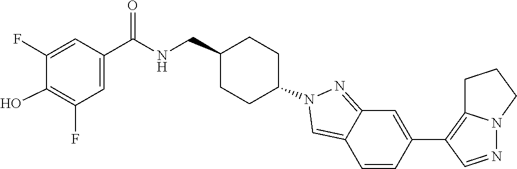 [SMILES: O=C(NC[C@H]1CC[C@H](n2cc3ccc(-c4cnn5c4CCC5)cc3n2)CC1)c1cc(F)c(O)c(F)c1] | N-({(1r,4r)-4-[6-(5,6-dihydro-4H- pyrrolo[1,2-b]pyrazol-3-yl)-2H- indazol-2-yl]cyclohexyl}methyl)- 3,5-difluoro-4- hydroxybenzamide |
| P65 | 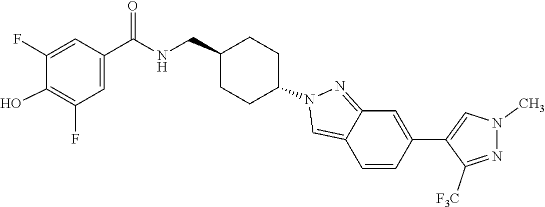 [SMILES: Cn1cc(-c2ccc3cn([C@H]4CC[C@H](CNC(=O)c5cc(F)c(O)c(F)c5)CC4)nc3c2)c(C(F)(F)F)n1] | 3,5-difluoro-4-hydroxy-N- {[(1r,4r)-4-{6-[1-methyl-3- (trifluoromethyl)-1H-pyrazol-4- yl]-2H-indazol-2- yl}cyclohexyl]methyl}benzamide |
| P66 | 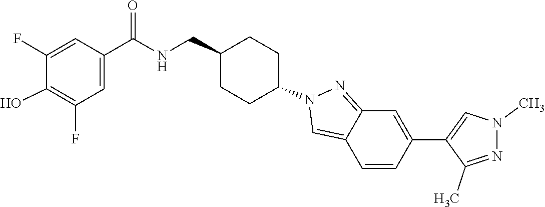 [SMILES: Cc1nn(C)cc1-c1ccc2cn([C@H]3CC[C@H](CNC(=O)c4cc(F)c(O)c(F)c4)CC3)nc2c1] | N-({(1r,4r)-4-[6-(1,3-dimethyl- 1H-pyrazol-4-yl)-2H-indazol-2- yl]cyclohexyl}methyl)-3,5- difluoro-4-hydroxybenzamide |
| P67 | 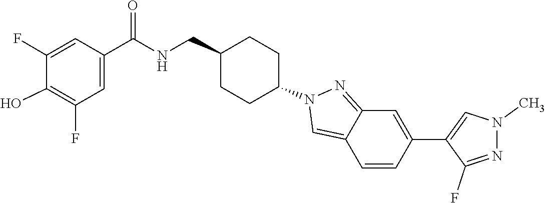 [SMILES: Cn1cc(-c2ccc3cn([C@H]4CC[C@H](CNC(=O)c5cc(F)c(O)c(F)c5)CC4)nc3c2)c(F)n1] | 3,5-difluoro-N-({(1r,4r)-4-[6-(3- fluoro-1-methyl-1H-pyrazol-4- yl)-2H-indazol-2- yl]cyclohexyl}methyl)-4- hydroxybenzamide |
| P68 | 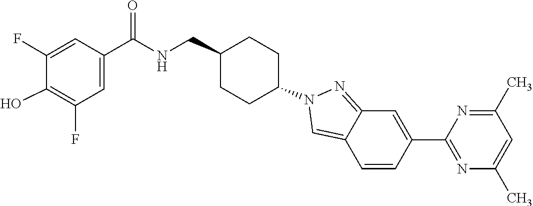 [SMILES: Cc1cc(C)nc(-c2ccc3cn([C@H]4CC[C@H](CNC(=O)c5cc(F)c(O)c(F)c5)CC4)nc3c2)n1] | N-({(1r,4r)-4-[6-(4,6- dimethylpyrimidin-2-yl)-2H- indazol-2-yl]cyclohexyl}methyl)- 3,5-difluoro-4- hydroxybenzamide |
| P69 | 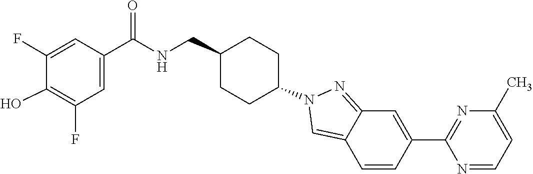 [SMILES: Cc1ccnc(-c2ccc3cn([C@H]4CC[C@H](CNC(=O)c5cc(F)c(O)c(F)c5)CC4)nc3c2)n1] | 3,5-difluoro-4-hydroxy-N- ({(1r,4r)-4-[6-(4- methylpyrimidin-2-yl)-2H- indazol-2- yl]cyclohexyl}methyl)benzamide |
| P70 | 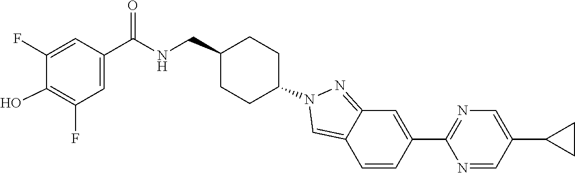 [SMILES: O=C(NC[C@H]1CC[C@H](n2cc3ccc(-c4ncc(C5CC5)cn4)cc3n2)CC1)c1cc(F)c(O)c(F)c1] | N-({(1r,4r)-4-[6-(5- cyclopropylpyrimidin-2-yl)-2H- indazol-2-yl]cyclohexyl}methyl)- 3,5-difluoro-4- hydroxybenzamide |
| P71 | 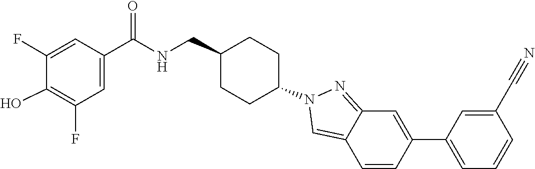 [SMILES: N#Cc1cccc(-c2ccc3cn([C@H]4CC[C@H](CNC(=O)c5cc(F)c(O)c(F)c5)CC4)nc3c2)c1] | N-({(1r,4r)-4-[6-(3- cyanophenyl)-2H-indazol-2- yl]cyclohexyl}methyl)-3,5- difluoro-4-hydroxybenzamide |
| P72 | 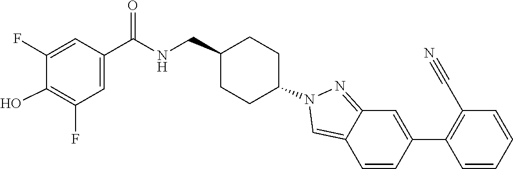 [SMILES: N#Cc1ccccc1-c1ccc2cn([C@H]3CC[C@H](CNC(=O)c4cc(F)c(O)c(F)c4)CC3)nc2c1] | N-({(1r,4r)-4-[6-(2- cyanophenyl)-2H-indazol-2- yl]cyclohexyl}methyl)-3,5- difluoro-4-hydroxybenzamide |
| P73 | 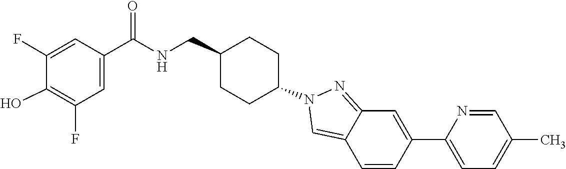 [SMILES: Cc1ccc(-c2ccc3cn([C@H]4CC[C@H](CNC(=O)c5cc(F)c(O)c(F)c5)CC4)nc3c2)nc1] | 3,5-difluoro-4-hydroxy-N- ({(1r,4r)-4-[6-(5-methylpyridin-2- yl)-2H-indazol-2- yl]cyclohexyl}methyl)benzamide |
| P74 | 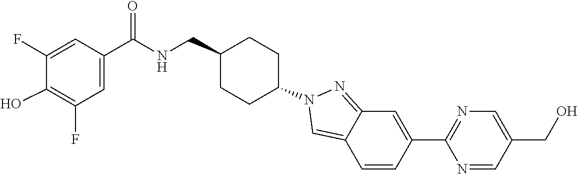 [SMILES: O=C(NC[C@H]1CC[C@H](n2cc3ccc(-c4ncc(CO)cn4)cc3n2)CC1)c1cc(F)c(O)c(F)c1] | 3,5-difluoro-4-hydroxy-N- {[(1r,4r)-4-{7-[5- (hydroxymethyl)pyrimidin-2-yl]- 2H-indazol-2- yl}cyclohexyl]methyl}benzamide |
| P75 | 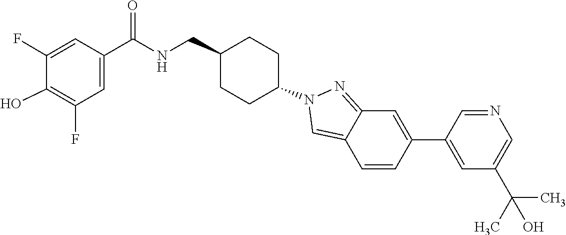 [SMILES: CC(C)(O)c1cncc(-c2ccc3cn([C@H]4CC[C@H](CNC(=O)c5cc(F)c(O)c(F)c5)CC4)nc3c2)c1] | 3,5-difluoro-4-hydroxy-N- {[(1r,4r)-4-{6-[5-(2- hydroxypropan-2-yl)pyridin-3- yl]-2H-indazol-2- yl}cyclohexyl]methyl}benzamide |
| P76 | 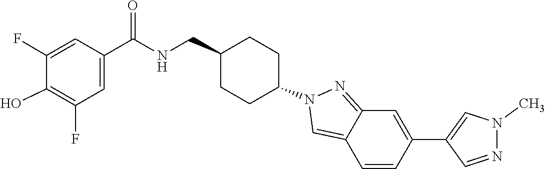 [SMILES: Cn1cc(-c2ccc3cn([C@H]4CC[C@H](CNC(=O)c5cc(F)c(O)c(F)c5)CC4)nc3c2)cn1] | 3,5-difluoro-4-hydroxy-N- ({(1r,4r)-4-[6-(1-methyl-1H- pyrazol-4-yl)-2H-indazol-2- yl]cyclohexyl}methyl)benzamide |
| P77 | 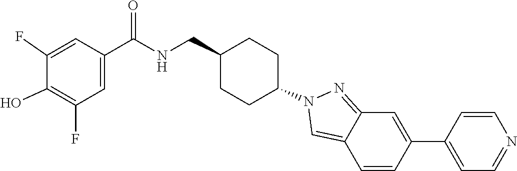 [SMILES: O=C(NC[C@H]1CC[C@H](n2cc3ccc(-c4ccncc4)cc3n2)CC1)c1cc(F)c(O)c(F)c1] | 3,5-difluoro-4-hydroxy-N- ({(1r,4r)-4-[6-(pyridin-4-yl)-2H- indazol-2- yl]cyclohexyl}methyl)benzamide |
| P78 | 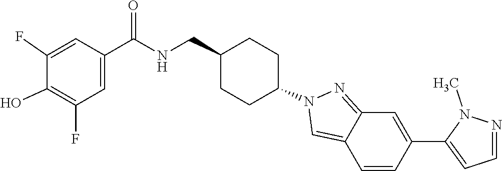 [SMILES: Cn1nccc1-c1ccc2cn([C@H]3CC[C@H](CNC(=O)c4cc(F)c(O)c(F)c4)CC3)nc2c1] | 3,5-difluoro-4-hydroxy-N- ({(1r,4r)-4-[6-(1-methyl-1H- pyrazol-5-yl)-2H-indazol-2- yl]cyclohexyl}methyl)benzamide |
| P79 | 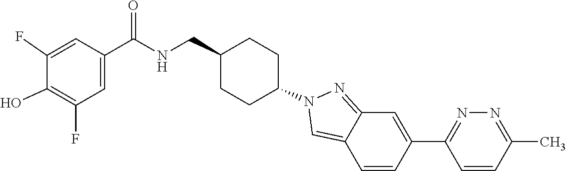 [SMILES: Cc1ccc(-c2ccc3cn([C@H]4CC[C@H](CNC(=O)c5cc(F)c(O)c(F)c5)CC4)nc3c2)nn1] | 3,5-difluoro-4-hydroxy-N- ({(1r,4r)-4-[6-(6- methylpyridazin-3-yl)-2H- indazol-2- yl]cyclohexyl}methyl)benzamide |
| P80 | 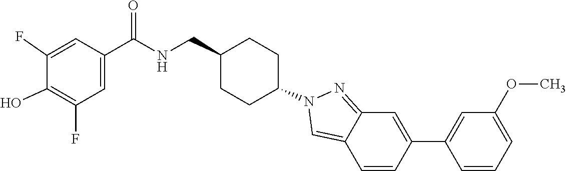 [SMILES: COc1cccc(-c2ccc3cn([C@H]4CC[C@H](CNC(=O)c5cc(F)c(O)c(F)c5)CC4)nc3c2)c1] | 3,5-difluoro-4-hydroxy-N- ({(1r,4r)-4-[6-(3- methoxyphenyl)-2H-indazol-2- yl]cyclohexyl}methyl)benzamide |
| P81 | 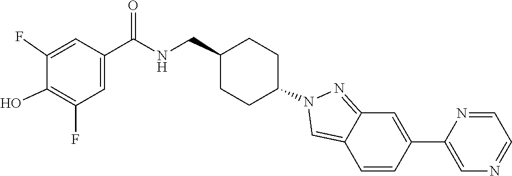 [SMILES: O=C(NC[C@H]1CC[C@H](n2cc3ccc(-c4cnccn4)cc3n2)CC1)c1cc(F)c(O)c(F)c1] | 3,5-difluoro-4-hydroxy-N- ({(1r,4r)-4-[6-(pyrazin-2-yl)-2H- indazol-2- yl]cyclohexyl}methyl)benzamide |
| P82 | 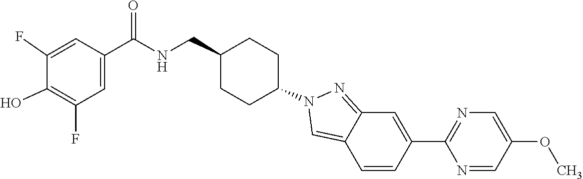 [SMILES: COc1cnc(-c2ccc3cn([C@H]4CC[C@H](CNC(=O)c5cc(F)c(O)c(F)c5)CC4)nc3c2)nc1] | 3,5-difluoro-4-hydroxy-N- ({(1r,4r)-4-[6-(5- methoxypyrimidin-2-yl)-2H- indazol-2- yl]cyclohexyl}methyl)benzamide |
| P83 | 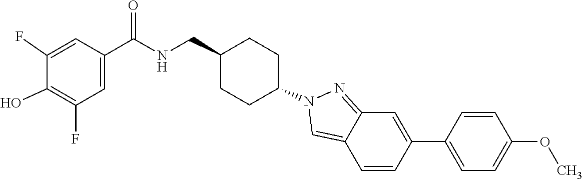 [SMILES: COc1ccc(-c2ccc3cn([C@H]4CC[C@H](CNC(=O)c5cc(F)c(O)c(F)c5)CC4)nc3c2)cc1] | 3,5-difluoro-4-hydroxy-N- ({(1r,4r)-4-[6-(4- methoxyphenyl)-2H-indazol-2- yl]cyclohexyl}methyl)benzamide |
| P84 | 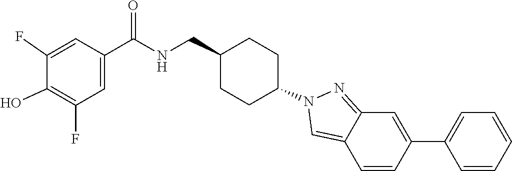 [SMILES: O=C(NC[C@H]1CC[C@H](n2cc3ccc(-c4ccccc4)cc3n2)CC1)c1cc(F)c(O)c(F)c1] | 3,5-difluoro-4-hydroxy-N- {[(1r,4r)-4-(6-phenyl-2H- indazol-2- yl)cyclohexyl]methyl}benzamide |
| P85 | 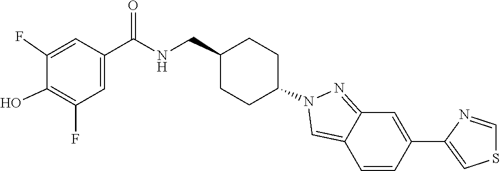 [SMILES: O=C(NC[C@H]1CC[C@H](n2cc3ccc(-c4cscn4)cc3n2)CC1)c1cc(F)c(O)c(F)c1] | 3,5-difluoro-4-hydroxy-N- ({(1r,4r)-4-[6-(1,3-thiazol-4-yl)- 2H-indazol-2- yl]cyclohexyl}methyl)benzamide |
| P86 | 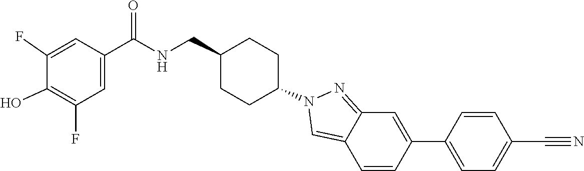 [SMILES: N#Cc1ccc(-c2ccc3cn([C@H]4CC[C@H](CNC(=O)c5cc(F)c(O)c(F)c5)CC4)nc3c2)cc1] | N-({(1r,4r)-4-[6-(4- cyanophenyl)-2H-indazol-2- yl]cyclohexyl}methyl)-3,5- difluoro-4-hydroxybenzamide |
| P87 | 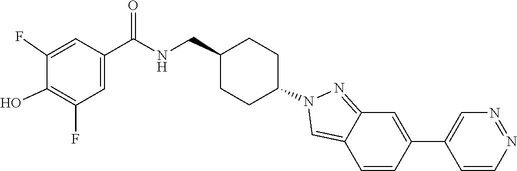 [SMILES: O=C(NC[C@H]1CC[C@H](n2cc3ccc(-c4ccnnc4)cc3n2)CC1)c1cc(F)c(O)c(F)c1] | 3,5-difluoro-4-hydroxy-N- ({(1r,4r)-4-[6-(pyridazin-4-yl)- 2H-indazol-2- yl]cyclohexyl}methyl)benzamide |
| P88 | 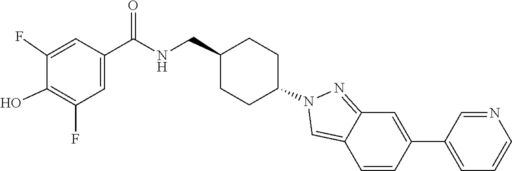 [SMILES: O=C(NC[C@H]1CC[C@H](n2cc3ccc(-c4cccnc4)cc3n2)CC1)c1cc(F)c(O)c(F)c1] | 3,5-difluoro-4-hydroxy-N- ({(1r,4r)-4-[6-(pyridin-3-yl)-2H- indazol-2- yl]cyclohexyl}methyl)benzamide |
| P89 | 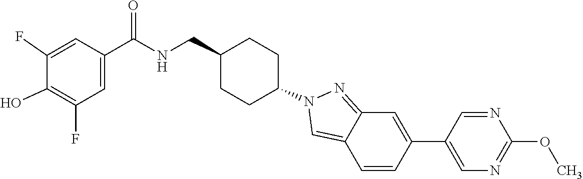 [SMILES: COc1ncc(-c2ccc3cn([C@H]4CC[C@H](CNC(=O)c5cc(F)c(O)c(F)c5)CC4)nc3c2)cn1] | 3,5-difluoro-4-hydroxy-N- ({(1r,4r)-4-[6-(2- methoxypyrimidin-5-yl)-2H- indazol-2- yl]cyclohexyl}methyl)benzamide |
| P90 | 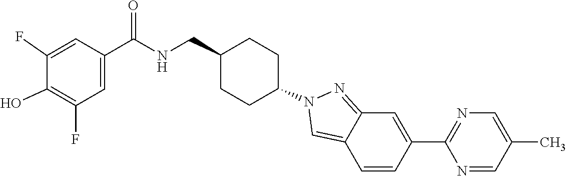 [SMILES: Cc1cnc(-c2ccc3cn([C@H]4CC[C@H](CNC(=O)c5cc(F)c(O)c(F)c5)CC4)nc3c2)nc1] | 3,5-difluoro-4-hydroxy-N- ({(1r,4r)-4-[6-(5- methylpyrimidin-2-yl)-2H- indazol-2- yl]cyclohexyl}methyl)benzamide |
| P91 | 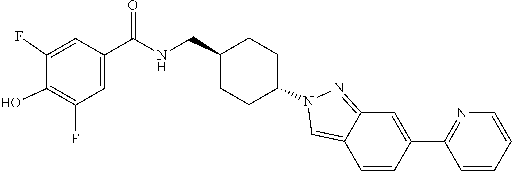 [SMILES: O=C(NC[C@H]1CC[C@H](n2cc3ccc(-c4ccccn4)cc3n2)CC1)c1cc(F)c(O)c(F)c1] | 3,5-difluoro-4-hydroxy-N- ({(1r,4r)-4-[6-(pyridin-2-yl)-2H- indazol-2- yl]cyclohexyl}methyl)benzamide |
| P92 | 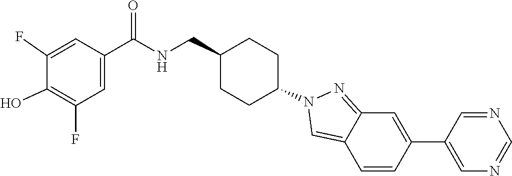 [SMILES: O=C(NC[C@H]1CC[C@H](n2cc3ccc(-c4cncnc4)cc3n2)CC1)c1cc(F)c(O)c(F)c1] | 3,5-difluoro-4-hydroxy-N- ({(1r,4r)-4-[6-(pyrimidin-5-yl)- 2H-indazol-2- yl]cyclohexyl}methyl)benzamide |
| P93 | 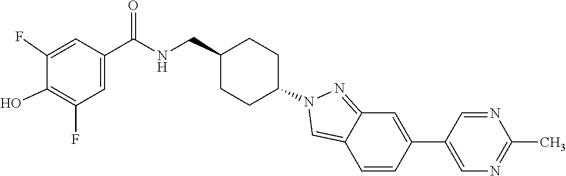 [SMILES: Cc1ncc(-c2ccc3cn([C@H]4CC[C@H](CNC(=O)c5cc(F)c(O)c(F)c5)CC4)nc3c2)cn1] | 3,5-difluoro-4-hydroxy-N- ({(1r,4r)-4-[6-(2- methylpyrimidin-5-yl)-2H- indazol-2- yl]cyclohexyl}methyl)benzamide |
| P94 | 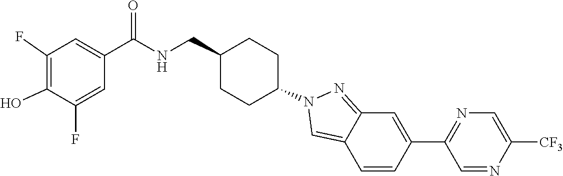 [SMILES: O=C(NC[C@H]1CC[C@H](n2cc3ccc(-c4cnc(C(F)(F)F)cn4)cc3n2)CC1)c1cc(F)c(O)c(F)c1] | 3,5-difluoro-4-hydroxy-N- {[(1r,4r)-4-{6-[5- (trifluoromethyl)pyrazin-2-yl]- 2H-indazol-2- yl}cyclohexyl]methyl}benzamide |
| P95 | 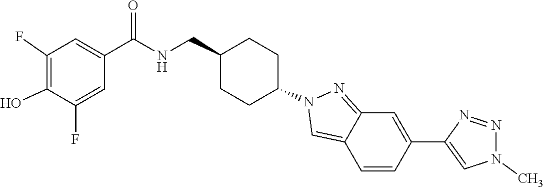 [SMILES: Cn1cc(-c2ccc3cn([C@H]4CC[C@H](CNC(=O)c5cc(F)c(O)c(F)c5)CC4)nc3c2)nn1] | 3,5-difluoro-4-hydroxy-N- ({(1r,4r)-4-[6-(1-methyl-1H- 1,2,3-triazol-4-yl)-2H-indazol-2- yl]cyclohexyl}methyl)benzamide |
| P96 | 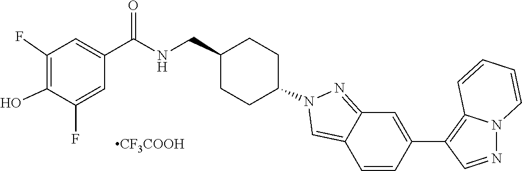 [SMILES: O=C(NC[C@H]1CC[C@H](n2cc3ccc(-c4cnn5ccccc45)cc3n2)CC1)c1cc(F)c(O)c(F)c1 ; "·CF3COOH"] | 3,5-difluoro-4-hydroxy-N- ({(1r,4r)-4-[6-(pyrazolo[1,5- a]pyridin-3-yl)-2H-indazol-2- yl]cyclohexyl}methyl)benzamide, trifluoroacetate salt |
| P97 | 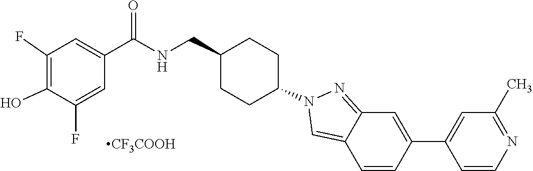 [SMILES: Cc1cc(-c2ccc3cn([C@H]4CC[C@H](CNC(=O)c5cc(F)c(O)c(F)c5)CC4)nc3c2)ccn1 ; "·CF3COOH"] | 3,5-difluoro-4-hydroxy-N- ({(1r,4r)-4-[6-(2-methylpyridin-4- yl)-2H-indazol-2- yl]cyclohexyl}methyl)benzamide, trifluoroacetate salt |
| P98 | 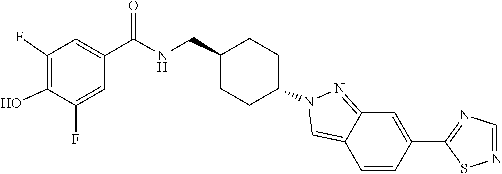 [SMILES: O=C(NC[C@H]1CC[C@H](n2cc3ccc(-c4ncns4)cc3n2)CC1)c1cc(F)c(O)c(F)c1] | 3,5-difluoro-4-hydroxy-N- ({(1r,4r)-4-[6-(1,2,4-thiadiazol-5- yl)-2H-indazol-2- yl]cyclohexyl}methyl)benzamide |
| P99 | 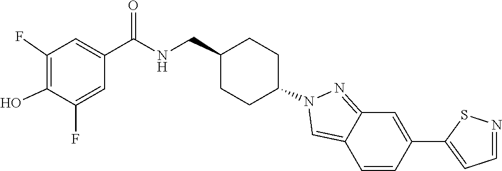 [SMILES: O=C(NC[C@H]1CC[C@H](n2cc3ccc(-c4ccns4)cc3n2)CC1)c1cc(F)c(O)c(F)c1] | 3,5-difluoro-4-hydroxy-N- ({(1r,4r)-4-[6-(1,2-thiazol-5-yl)- 2H-indazol-2- yl]cyclohexyl}methyl)benzamide |
| P100 | 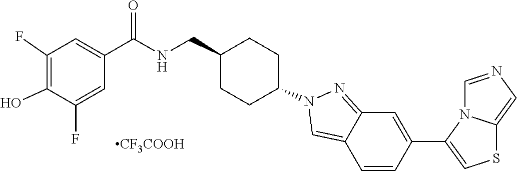 | 3,5-difluoro-4-hydroxy-N- ({(1r,4r)-4-[6-(imidazo[5,1- b][1,3-thiazol-3-yl)-2H-indazol- 2- yl]cyclohexyl}methyl)benzamide, trifluoroacetate salt |
| P101 | 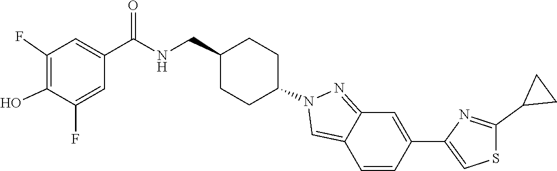 [SMILES: O=C(NC[C@H]1CC[C@H](n2cc3ccc(-c4csc(C5CC5)n4)cc3n2)CC1)c1cc(F)c(O)c(F)c1] | N-({(1r,4r)-4-[6-(2-cyclopropyl- 1,3-thiazol-4-yl)-2H-indazol-2- yl]cyclohexyl}methyl)-3,5- difluoro-4-hydroxybenzamide |
| P102 | 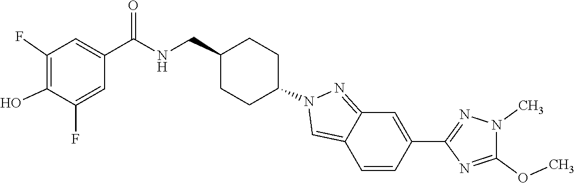 [SMILES: COc1nc(-c2ccc3cn([C@H]4CC[C@H](CNC(=O)c5cc(F)c(O)c(F)c5)CC4)nc3c2)nn1C] | 3,5-difluoro-4-hydroxy-N- ({(1r,4r)-4-[6-(5-methoxy-1- methyl-1H-1,2,4-triazol-3-yl)- 2H-indazol-2- yl]cyclohexyl}methyl)benzamide |
| P103 | 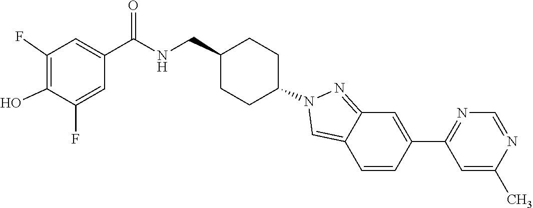 [SMILES: Cc1cc(-c2ccc3cn([C@H]4CC[C@H](CNC(=O)c5cc(F)c(O)c(F)c5)CC4)nc3c2)ncn1] | 3,5-difluoro-4-hydroxy-N- ({(1r,4r)-4-[6-(6- methylpyrimidin-4-yl)-2H- indazol-2- yl]cyclohexyl}methyl)benzamide |
| P104 | 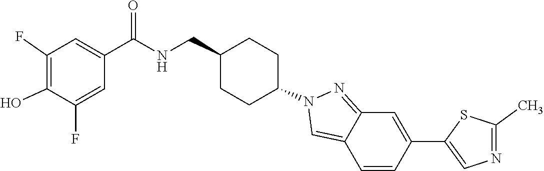 [SMILES: Cc1ncc(-c2ccc3cn([C@H]4CC[C@H](CNC(=O)c5cc(F)c(O)c(F)c5)CC4)nc3c2)s1] | 3,5-difluoro-4-hydroxy-N- ({(1r,4r)-4-[6-(2-methyl-1,3- thiazol-5-yl)-2H-indazol-2- yl]cyclohexyl}methyl)benzamide |
| P105 | 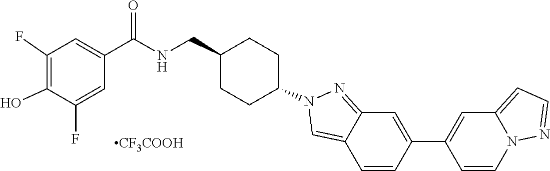 [SMILES: O=C(NC[C@H]1CC[C@H](n2cc3ccc(-c4ccn5nccc5c4)cc3n2)CC1)c1cc(F)c(O)c(F)c1 ; "·CF3COOH"] | 3,5-difluoro-4-hydroxy-N- ({(1r,4r)-4-[6-(pyrazolo[1,5- a]pyridin-5-yl)-2H-indazol-2- yl]cyclohexyl}methyl)benzamide, trifluoroacetate salt |
| P106 | 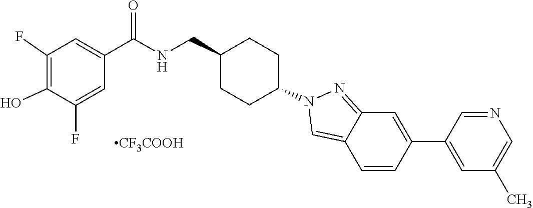 [SMILES: Cc1cncc(-c2ccc3cn([C@H]4CC[C@H](CNC(=O)c5cc(F)c(O)c(F)c5)CC4)nc3c2)c1 ; "·CF3COOH"] | 3,5-difluoro-4-hydroxy-N- ({(1r,4r)-4-[6-(5-methylpyridin-3- yl)-2H-indazol-2- yl]cyclohexyl}methyl)benzamide, trifluoroacetate salt |
| P107 | 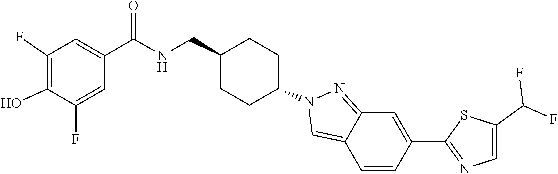 [SMILES: O=C(NC[C@H]1CC[C@H](n2cc3ccc(-c4ncc(C(F)F)s4)cc3n2)CC1)c1cc(F)c(O)c(F)c1] | N-{[(1r,4r)-4-{6-[5- (difluoromethyl)-1,3-thiazol-2- yl]-2H-indazol-2- yl}cyclohexyl]methyl}-3,5- difluoro-4-hydroxybenzamide |
| P108 | 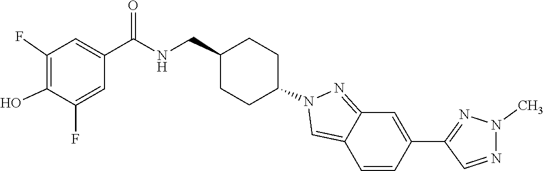 [SMILES: Cn1ncc(-c2ccc3cn([C@H]4CC[C@H](CNC(=O)c5cc(F)c(O)c(F)c5)CC4)nc3c2)n1] | 3,5-difluoro-4-hydroxy-N- ({(1r,4r)-4-[6-(2-methyl-2H- 1,2,3-triazol-4-yl)-2H-indazol-2- yl]cyclohexyl}methyl)benzamide |
| P109 | 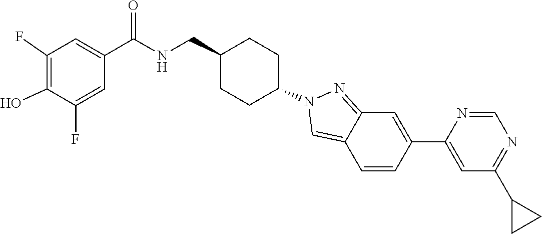 [SMILES: O=C(NC[C@H]1CC[C@H](n2cc3ccc(-c4cc(C5CC5)ncn4)cc3n2)CC1)c1cc(F)c(O)c(F)c1] | N-({(1r,4r)-4-[6-(6- cyclopropylpyrimidin-4-yl)-2H- indazol-2-yl]cyclohexyl}methyl)- 3,5-difluoro-4- hydroxybenzamide |
| P110 | 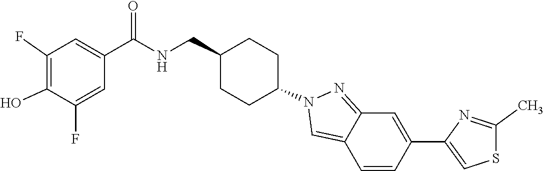 [SMILES: Cc1nc(-c2ccc3cn([C@H]4CC[C@H](CNC(=O)c5cc(F)c(O)c(F)c5)CC4)nc3c2)cs1] | 3,5-difluoro-4-hydroxy-N- ({(1r,4r)-4-[6-(2-methyl-1,3- thiazol-4-yl)-2H-indazol-2- yl]cyclohexyl}methyl)benzamide |
| P111 | 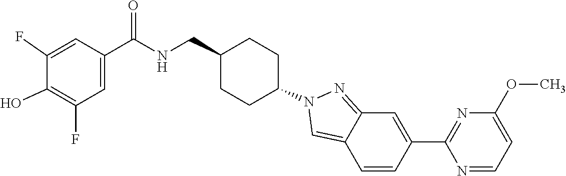 [SMILES: COc1ccnc(-c2ccc3cn([C@H]4CC[C@H](CNC(=O)c5cc(F)c(O)c(F)c5)CC4)nc3c2)n1] | 3,5-difluoro-4-hydroxy-N- ({(1r,4r)-4-[6-(4- methoxypyrimidin-2-yl)-2H- indazol-2- yl]cyclohexyl}methyl)benzamide |
| P112 | 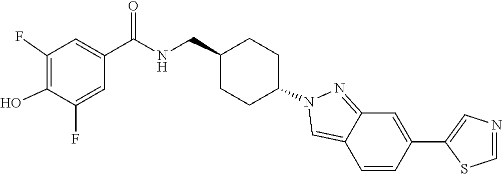 [SMILES: O=C(NC[C@H]1CC[C@H](n2cc3ccc(-c4cncs4)cc3n2)CC1)c1cc(F)c(O)c(F)c1] | 3,5-difluoro-4-hydroxy-N- ({(1r,4r)-4-[6-(1,3-thiazol-5-yl)- 2H-indazol-2- yl]cyclohexyl}methyl)benzamide |
| P113 | 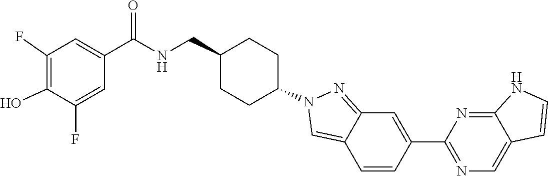 [SMILES: O=C(NC[C@H]1CC[C@H](n2cc3ccc(-c4ncc5cc[nH]c5n4)cc3n2)CC1)c1cc(F)c(O)c(F)c1] | 3,5-difluoro-4-hydroxy-N- ({(1r,4r)-4-[6-(7H-pyrrolo[2,3- d]pyrimidin-2-yl)-2H-indazol-2- yl]cyclohexyl}methyl)benzamide |
| P114 | 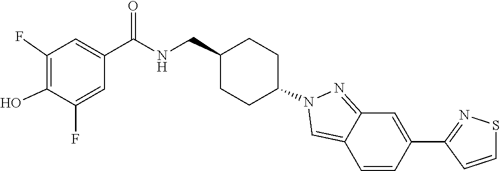 [SMILES: O=C(NC[C@H]1CC[C@H](n2cc3ccc(-c4ccsn4)cc3n2)CC1)c1cc(F)c(O)c(F)c1] | 3,5-difluoro-4-hydroxy-N- ({(1r,4r)-4-[6-(1,2-thiazol-3-yl)- 2H-indazol-2- yl]cyclohexyl}methyl)benzamide |
| P115 | 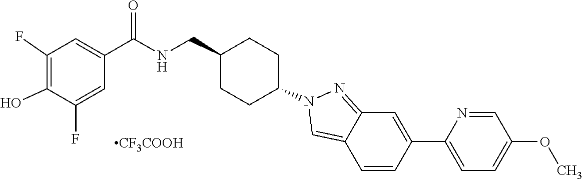 [SMILES: COc1ccc(-c2ccc3cn([C@H]4CC[C@H](CNC(=O)c5cc(F)c(O)c(F)c5)CC4)nc3c2)nc1 ; "·CF3COOH"] | 3,5-difluoro-4-hydroxy-N- ({(1r,4r)-4-[6-(5-methoxypyridin- 2-yl)-2H-indazol-2- yl]cyclohexyl}methyl)benzamide, trifluoroactate salt |
| P116 | 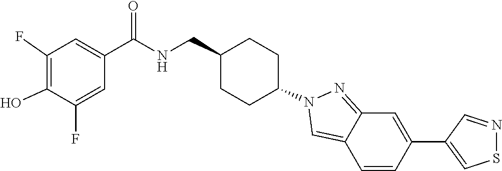 [SMILES: O=C(NC[C@H]1CC[C@H](n2cc3ccc(-c4cnsc4)cc3n2)CC1)c1cc(F)c(O)c(F)c1] | 3,5-difluoro-4-hydroxy-N- ({(1r,4r)-4-[6-(1,2-thiazol-4-yl)- 2H-indazol-2- yl]cyclohexyl}methyl)benzamide |
| P117 | 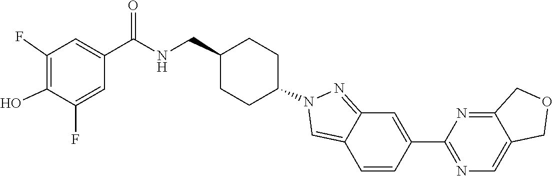 [SMILES: O=C(NC[C@H]1CC[C@H](n2cc3ccc(-c4ncc5c(n4)COC5)cc3n2)CC1)c1cc(F)c(O)c(F)c1] | N-({(1r,4r)-4-[6-(5,7- dihydrofuro[3,4-d]pyrimidin-2- yl)-2H-indazol-2- yl]cyclohexyl}methyl)-3,5- difluoro-4-hydroxybenzamide |
| P118 | 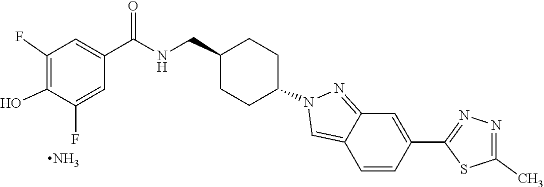 | 3,5-difluoro-4-hydroxy-N- ({(1r,4r)-4-[6-(5-methyl-1,3,4- thiadiazol-2-yl)-2H-indazol-2- yl]cyclohexyl}methyl)benzamide, ammonium salt |
| P119 | 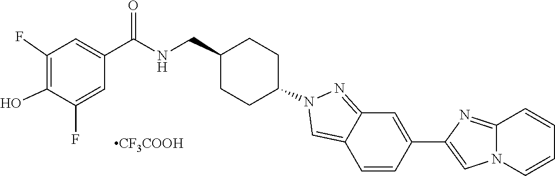 [SMILES: O=C(NC[C@H]1CC[C@H](n2cc3ccc(-c4cn5ccccc5n4)cc3n2)CC1)c1cc(F)c(O)c(F)c1 ; "·CF3COOH"] | 3,5-difluoro-4-hydroxy-N- ({(1r,4r)-4-[6-(imidazo[1,2- a]pyridin-2-yl)-2H-indazol-2- yl]cyclohexyl}methyl)benzamide, trifluoroacetate salt |
| P120 | 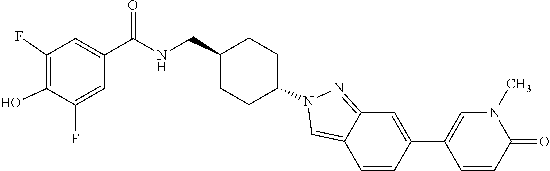 [SMILES: Cn1cc(-c2ccc3cn([C@H]4CC[C@H](CNC(=O)c5cc(F)c(O)c(F)c5)CC4)nc3c2)ccc1=O] | 3,5-difluoro-4-hydroxy-N- ({(1r,4r)-4-[6-(1-methyl-6-oxo- 1,6-dihydropyridin-3-yl)-2H- indazol-2- yl]cyclohexyl}methyl)benzamide |
| P121 | 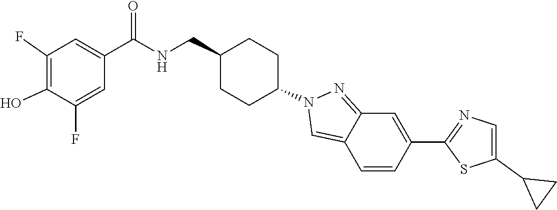 [SMILES: O=C(NC[C@H]1CC[C@H](n2cc3ccc(-c4ncc(C5CC5)s4)cc3n2)CC1)c1cc(F)c(O)c(F)c1] | N-({(1r,4r)-4-[6-(5-cyclopropyl- 1,3-thiazol-2-yl)-2H-indazol-2- yl]cyclohexyl}methyl)-3,5- difluoro-4-hydroxybenzamide |
| P122 | 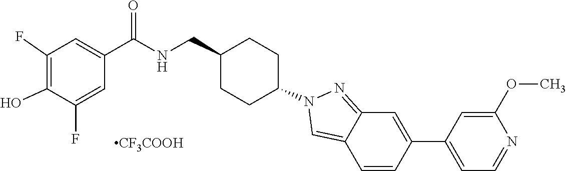 [SMILES: COc1cc(-c2ccc3cn([C@H]4CC[C@H](CNC(=O)c5cc(F)c(O)c(F)c5)CC4)nc3c2)ccn1 ; "·CF3COOH"] | 3,5-difluoro-4-hydroxy-N- ({(1r,4r)-4-[6-(2-methoxypyridin- 4-yl)-2H-indazol-2- yl]cyclohexyl}methyl)benzamide, trifluoroacetate salt |
| P123 | 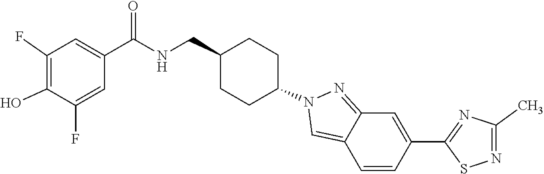 [SMILES: Cc1nsc(-c2ccc3cn([C@H]4CC[C@H](CNC(=O)c5cc(F)c(O)c(F)c5)CC4)nc3c2)n1] | 3,5-difluoro-4-hydroxy-N- ({(1r,4r)-4-[6-(3-methyl-1,2,4- thiadiazol-5-yl)-2H-indazol-2- yl]cyclohexyl}methyl)benzamide |
| P124 | 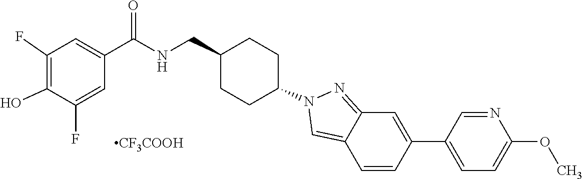 [SMILES: COc1ccc(-c2ccc3cn([C@H]4CC[C@H](CNC(=O)c5cc(F)c(O)c(F)c5)CC4)nc3c2)cn1 ; "·CF3COOH"] | 3,5-difluoro-4-hydroxy-N- ({(1r,4r)-4-[6-(6-methoxypyridin- 3-yl)-2H-indazol-2- yl]cyclohexyl}methyl)benzamide, trifluoroacetate salt |
| P125 | 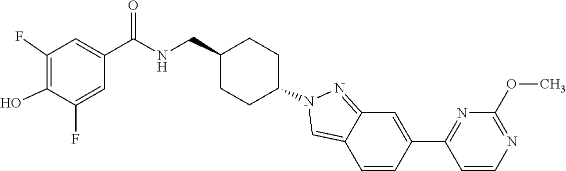 [SMILES: COc1nccc(-c2ccc3cn([C@H]4CC[C@H](CNC(=O)c5cc(F)c(O)c(F)c5)CC4)nc3c2)n1] | 3,5-difluoro-4-hydroxy-N- ({(1r,4r)-4-[6-(2- methoxypyrimidin-4-yl)-2H- indazol-2- yl]cyclohexyl}methyl)benzamide |
| P126 | 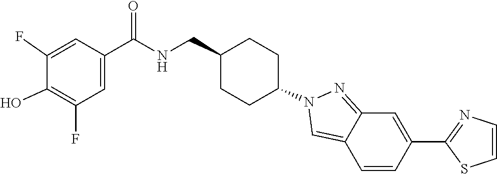 [SMILES: O=C(NC[C@H]1CC[C@H](n2cc3ccc(-c4nccs4)cc3n2)CC1)c1cc(F)c(O)c(F)c1] | 3,5-difluoro-4-hydroxy-N- ({(1r,4r)-4-[6-(1,3-thiazol-2-yl)- 2H-indazol-2- yl]cyclohexyl}methyl)benzamide |
| P127 | 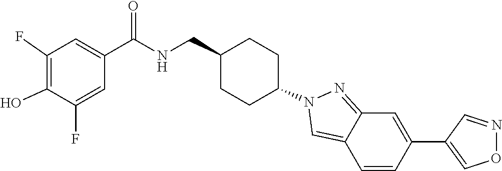 [SMILES: O=C(NC[C@H]1CC[C@H](n2cc3ccc(-c4cnoc4)cc3n2)CC1)c1cc(F)c(O)c(F)c1] | 3,5-difluoro-4-hydroxy-N- ({(1r,4r)-4-[6-(1,2-oxazol-4-yl)- 2H-indazol-2- yl]cyclohexyl}methyl)benzamide |
| P128 | 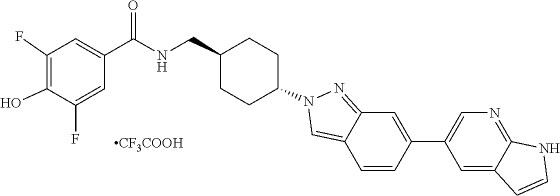 [SMILES: O=C(NC[C@H]1CC[C@H](n2cc3ccc(-c4cnc5[nH]ccc5c4)cc3n2)CC1)c1cc(F)c(O)c(F)c1 ; "·CF3COOH"] | 3,5-difluoro-4-hydroxy-N- ({(1r,4r)-4-[6-(1H-pyrrolo[2,3- b]pyridin-5-yl)-2H-indazol-2- yl]cyclohexyl}methyl)benzamide, trifluoroacetate salt |
| P129 | 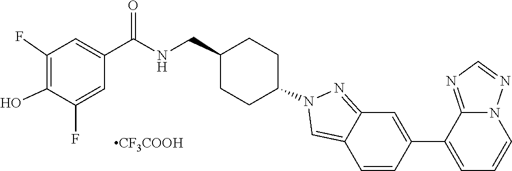 [SMILES: O=C(NC[C@H]1CC[C@H](n2cc3ccc(-c4cccn5ncnc45)cc3n2)CC1)c1cc(F)c(O)c(F)c1 ; "·CF3COOH"] | 3,5-difluoro-4-hydroxy-N- ({(1r,4r)-4-[6-([1,2,4]triazolo[1,5- a]pyridin-8-yl)-2H-indazol-2- yl]cyclohexyl}methyl)benzamide, trifluoroacetate salt |
| P130 | 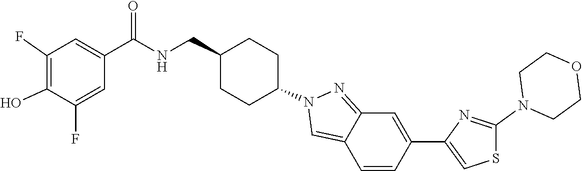 [SMILES: O=C(NC[C@H]1CC[C@H](n2cc3ccc(-c4csc(N5CCOCC5)n4)cc3n2)CC1)c1cc(F)c(O)c(F)c1] | 3,5-difluoro-4-hydroxy-N- {[(1r,4r)-4-{6-[2-(morpholin-4- yl)-1,3-thiazol-4-yl]-2H-indazol- 2- yl}cyclohexyl]methyl}benzamide |
| P131 | 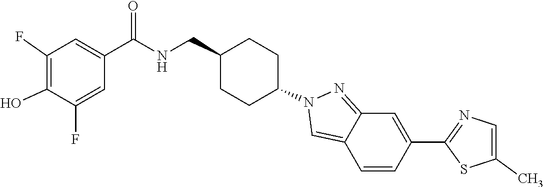 [SMILES: Cc1cnc(-c2ccc3cn([C@H]4CC[C@H](CNC(=O)c5cc(F)c(O)c(F)c5)CC4)nc3c2)s1] | 3,5-difluoro-4-hydroxy-N- ({(1r,4r)-4-[6-(5-methyl-1,3- thiazol-2-yl)-2H-indazol-2- yl]cyclohexyl}methyl)benzamide |
| P132 | 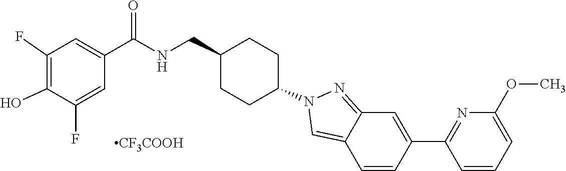 [SMILES: COc1cccc(-c2ccc3cn([C@H]4CC[C@H](CNC(=O)c5cc(F)c(O)c(F)c5)CC4)nc3c2)n1 ; "·CF3COOH"] | 3,5-difluoro-4-hydroxy-N- ({(1r,4r)-4-[6-(6-methoxypyridin- 2-yl)-2H-indazol-2- yl]cyclohexyl}methyl)benzamide, trifluoroacetate salt |
| P133 | 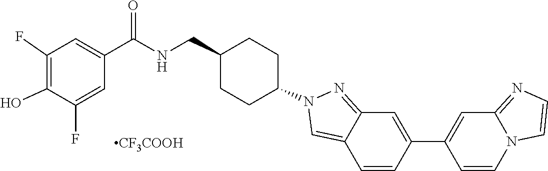 [SMILES: O=C(NC[C@H]1CC[C@H](n2cc3ccc(-c4ccn5ccnc5c4)cc3n2)CC1)c1cc(F)c(O)c(F)c1 ; "·CF3COOH"] | 3,5-difluoro-4-hydroxy-N- ({(1r,4r)-4-[6-(imidazo[1,2- a]pyridin-7-yl)-2H-indazol-2- yl]cyclohexyl}methyl)benzamide, trifluoroacetate salt |
| P134 | 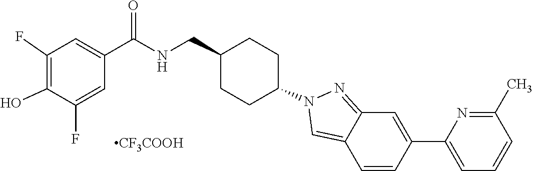 [SMILES: Cc1cccc(-c2ccc3cn([C@H]4CC[C@H](CNC(=O)c5cc(F)c(O)c(F)c5)CC4)nc3c2)n1 ; "·CF3COOH"] | 3,5-difluoro-4-hydroxy-N- ({(1r,4r)-4-[6-(6-methylpyridin-2- yl)-2H-indazol-2- yl]cyclohexyl}methyl)benzamide, trifluoroacetate salt |
| P135 | 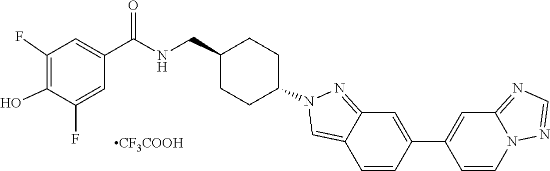 [SMILES: O=C(NC[C@H]1CC[C@H](n2cc3ccc(-c4ccn5ncnc5c4)cc3n2)CC1)c1cc(F)c(O)c(F)c1 ; "·CF3COOH"] | 3,5-difluoro-4-hydroxy-N- ({(1r,4r)-4-[6-([1,2,4]triazolo[1,5- a]pyridin-7-yl)-2H-indazol-2- yl]cyclohexyl}methyl)benzamide, trifluoroacetate salt |
| P136 | 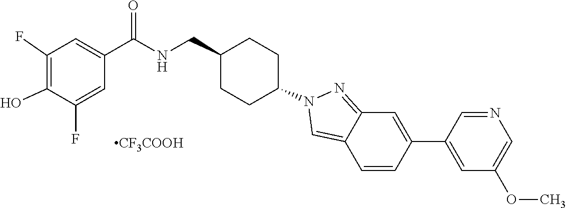 [SMILES: COc1cncc(-c2ccc3cn([C@H]4CC[C@H](CNC(=O)c5cc(F)c(O)c(F)c5)CC4)nc3c2)c1 ; "·CF3COOH"] | 3,5-difluoro-4-hydroxy-N- ({(1r,4r)-4-[6-(5-methoxypyridin- 3-yl)-2H-indazol-2- yl]cyclohexyl}methyl)benzamide, trifluoroacetate salt |
| P137 | 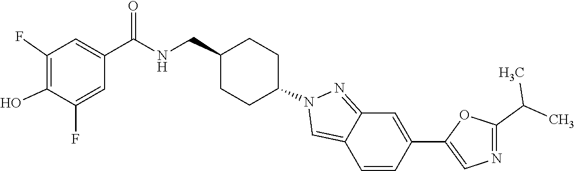 [SMILES: CC(C)c1ncc(-c2ccc3cn([C@H]4CC[C@H](CNC(=O)c5cc(F)c(O)c(F)c5)CC4)nc3c2)o1] | 3,5-difluoro-4-hydroxy-N- {[(1r,4r)-4-{6-[2-(propan-2-yl)- 1,3-oxazol-5-yl]-2H-indazol-2- yl}cyclohexyl]methyl}benzamide |
| P138 | 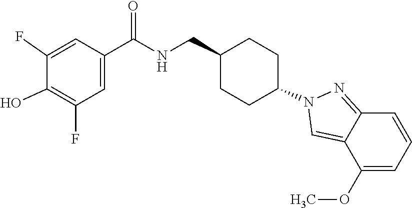 [SMILES: COc1cccc2nn([C@H]3CC[C@H](CNC(=O)c4cc(F)c(O)c(F)c4)CC3)cc12] | 3,5-difluoro-4-hydroxy-N- {[(1r,4r)-4-(4-methoxy-2H- indazol-2- yl)cyclohexyl]methyl}benzamide |
| P139 | 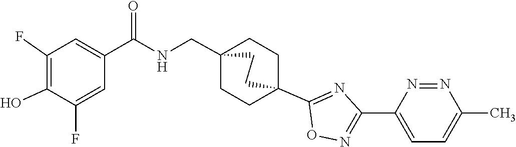 [SMILES: Cc1ccc(-c2noc([C@]34CC[C@](CNC(=O)c5cc(F)c(O)c(F)c5)(CC3)CC4)n2)nn1] | 3,5-difluoro-4-hydroxy-N-({4-[3- (6-methylpyridazin-3-yl)-1,2,4- oxadiazol-5- yl]bicyclo[2.2.2]octan-1- yl}methyl)benzamide |
| P140 | 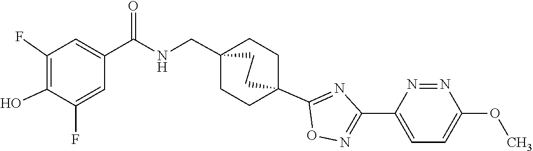 [SMILES: COc1ccc(-c2noc([C@]34CC[C@](CNC(=O)c5cc(F)c(O)c(F)c5)(CC3)CC4)n2)nn1] | 3,5-difluoro-4-hydroxy-N-({4-[3- (6-methoxypyridazin-3-yl)- 1,2,4-oxadiazol-5- yl]bicyclo[2.2.2]octan-1- yl}methyl)benzamide |
| P141 | 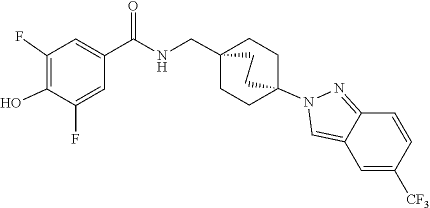 [SMILES: O=C(NC[C@]12CC[C@](n3cc4cc(C(F)(F)F)ccc4n3)(CC1)CC2)c1cc(F)c(O)c(F)c1] | 3,5-difluoro-4-hydroxy-N-({4-[5- (trifluoromethyl)-2H-indazol-2- yl]bicyclo[2.2.2]octan-1- yl}methyl)benzamide |
| P142 | 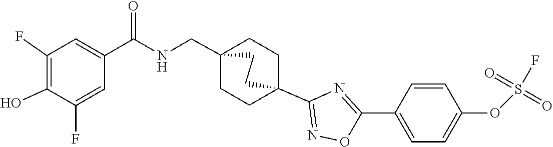 [SMILES: O=C(NC[C@]12CC[C@](c3noc(-c4ccc(OS(=O)(=O)F)cc4)n3)(CC1)CC2)c1cc(F)c(O)c(F)c1] | 4-(3-{4-[(3,5-difluoro-4- hydroxybenzamido)methyl] bicyclo[2.2.2]octan-1-yl}-1,2,4- oxadiazol-5-yl)phenyl sulfurofluoridate |
| P143 | 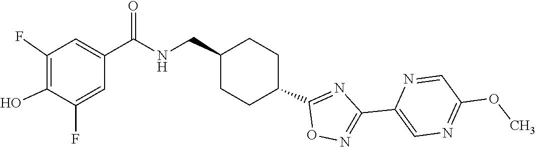 [SMILES: COc1cnc(-c2noc([C@H]3CC[C@H](CNC(=O)c4cc(F)c(O)c(F)c4)CC3)n2)cn1] | 3,5-difluoro-4-hydroxy-N- ({(1r,4r)-4-[3-(5- methoxypyrazin-2-yl)-1,2,4- oxadiazol-5- yl]cyclohexyl}methyl)benzamide |
| P144 | 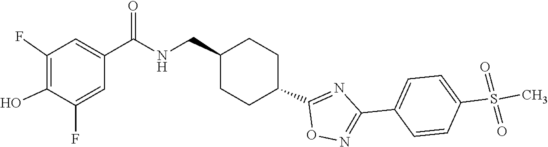 [SMILES: CS(=O)(=O)c1ccc(-c2noc([C@H]3CC[C@H](CNC(=O)c4cc(F)c(O)c(F)c4)CC3)n2)cc1] | 3,5-difluoro-4-hydroxy-N- {[(1r,4r)-4-{3-[4- (methanesulfonyl)phenyl]-1,2,4- oxadiazol-5- yl}cyclohexyl]methyl}benzamide |
| P145 | 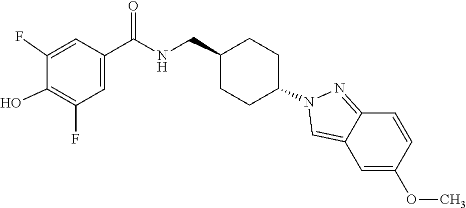 [SMILES: COc1ccc2nn([C@H]3CC[C@H](CNC(=O)c4cc(F)c(O)c(F)c4)CC3)cc2c1] | 3,5-difluoro-4-hydroxy-N- {[(1r,4r)-4-(5-methoxy-2H- indazol-2- yl)cyclohexyl]methyl}benzamide |
| P146 | 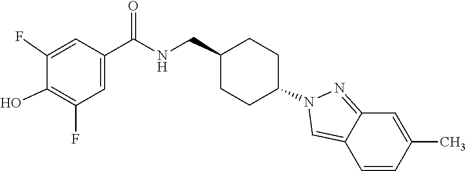 [SMILES: Cc1ccc2cn([C@H]3CC[C@H](CNC(=O)c4cc(F)c(O)c(F)c4)CC3)nc2c1] | 3,5-difluoro-4-hydroxy-N- {[(1r,4r)-4-(6-methyl-2H- indazol-2- yl)cyclohexyl]methyl}benzamide |
| P147 | 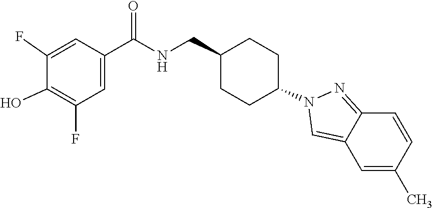 [SMILES: Cc1ccc2nn([C@H]3CC[C@H](CNC(=O)c4cc(F)c(O)c(F)c4)CC3)cc2c1] | 3,5-difluoro-4-hydroxy-N- {[(1r,4r)-4-(5-methyl-2H- indazol-2- yl)cyclohexyl]methyl}benzamide |
| P148 | 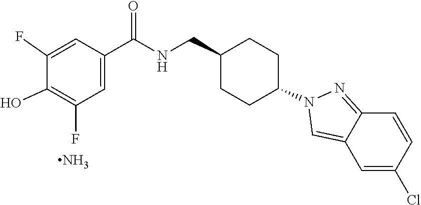 | N-{[(1r,4r)-4-(5-chloro-2H- indazol-2-yl)cyclohexyl]methyl}- 3,5-difluoro-4- hydroxybenzamide, ammonium salt |
| P149 | 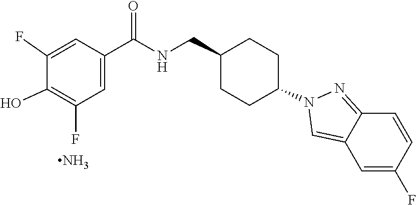 | 3,5-difluoro-N-{[(1r,4r)-4-(5- fluoro-2H-indazol-2- yl)cyclohexyl]methyl}-4- hydroxybenzamide, ammonium salt |
| P150 | 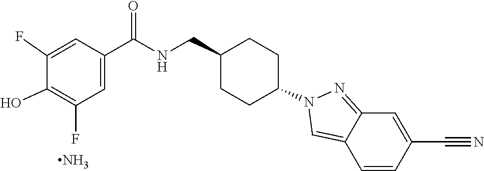 | N-{[(1r,4r)-4-(6-cyano-2H- indazol-2-yl)cyclohexyl]methyl}- 3,5-difluoro-4- hydroxybenzamide, ammonium salt |
| P151 | 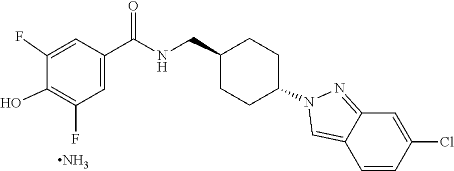 | N-{[(1r,4r)-4-(6-chloro-2H- indazol-2-yl)cyclohexyl]methyl}- 3,5-difluoro-4- hydroxybenzamide, ammonium salt |
| P152 | 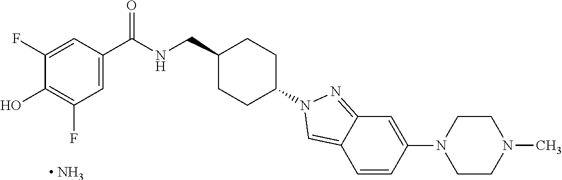 | 3,5-difluoro-4-hydroxy-N- ({(1r,4r)-4-[6-(4- methylpiperazin-1-yl)-2H- indazol-2- yl]cyclohexyl}methyl)benzamide, ammonium salt |
| P153 | 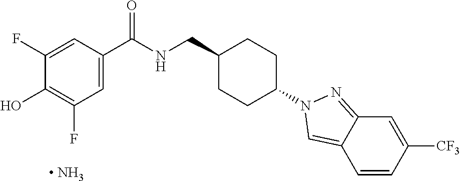 | 3,5-difluoro-4-hydroxy-N- ({(1r,4r)-4-[6-(trifluoromethyl)- 2H-indazol-2- yl]cyclohexyl}methyl)benzamide, ammonium salt |
| P154 | 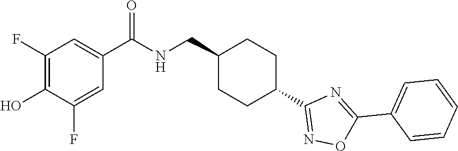 [SMILES: O=C(NC[C@H]1CC[C@H](c2noc(-c3ccccc3)n2)CC1)c1cc(F)c(O)c(F)c1] | 3,5-difluoro-4-hydroxy-N- {[(1r,4r)-4-(5-phenyl-1,2,4- oxadiazol-3- yl)cyclohexyl]methyl}benzamide |
| P155 | 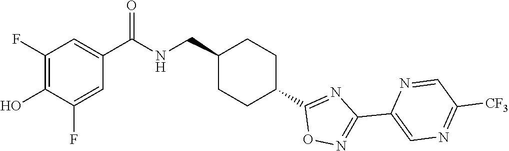 [SMILES: O=C(NC[C@H]1CC[C@H](c2nc(-c3cnc(C(F)(F)F)cn3)no2)CC1)c1cc(F)c(O)c(F)c1] | 3,5-difluoro-4-hydroxy-N- {[(1r,4r)-4-{3-[5- (trifluoromethyl)pyrazin-2-yl]- 1,2,4-oxadiazol-5- yl}cyclohexyl]methyl}benzamide |
| P156 | 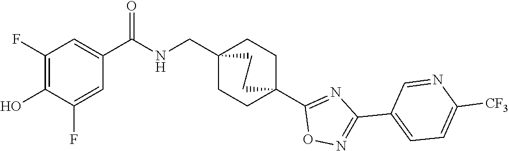 [SMILES: O=C(NC[C@]12CC[C@](c3nc(-c4ccc(C(F)(F)F)nc4)no3)(CC1)CC2)c1cc(F)c(O)c(F)c1] | 3,5-difluoro-4-hydroxy-N-[(4-{3- [6-(trifluoromethyl)pyridin-3-yl]- 1,2,4-oxadiazol-5- yl}bicyclo[2.2.2]octan-1- yl)methyl]benzamide |
| P157 | 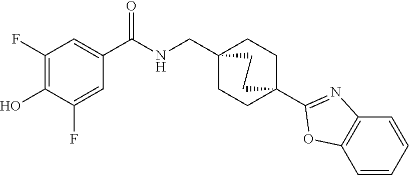 [SMILES: O=C(NC[C@]12CC[C@](c3nc4ccccc4o3)(CC1)CC2)c1cc(F)c(O)c(F)c1] | N-{[4-(1,3-benzoxazol-2- yl)bicyclo[2.2.2]octan-1- yl]methyl}-3,5-difluoro-4- hydroxybenzamide |
| P158 | 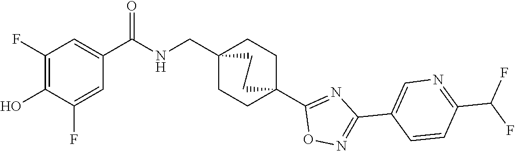 [SMILES: O=C(NC[C@]12CC[C@](c3nc(-c4ccc(C(F)F)nc4)no3)(CC1)CC2)c1cc(F)c(O)c(F)c1] | N-[(4-{3-[6- (difluoromethyl)pyridin-3-yl]- 1,2,4-oxadiazol-5- yl}bicyclo[2.2.2]octan-1- yl)methyl]-3,5-difluoro-4- hydroxybenzamide |
| P159 | 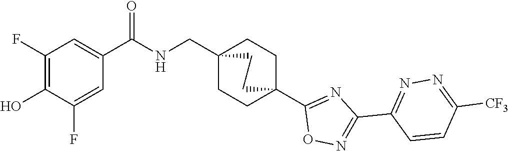 [SMILES: O=C(NC[C@]12CC[C@](c3nc(-c4ccc(C(F)(F)F)nn4)no3)(CC1)CC2)c1cc(F)c(O)c(F)c1] | 3,5-difluoro-4-hydroxy-N-[(4-{3- [6-(trifluoromethyl)pyridazin-3- yl]-1,2,4-oxadiazol-5- yl}bicyclo[2.2.2]octan-1- yl)methyl]benzamide |
| P160 | 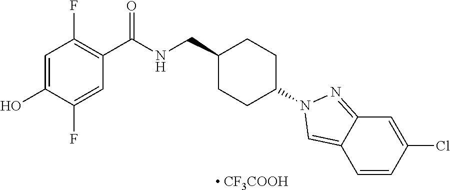 | N-{[(1r,4r)-4-(6-chloro-2H- indazol-2-yl)cyclohexyl]methyl}- 2,5-difluoro-4- hydroxybenzamide, trifluoroacetate salt |
| P161 | 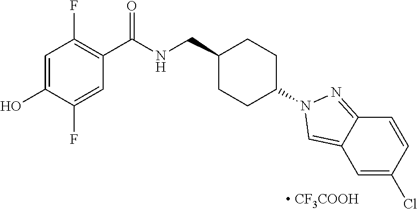 | N-{[(1r,4r)-4-(5-chloro-2H- indazol-2-yl)cyclohexyl]methyl}- 2,5-difluoro-4- hydroxybenzamide, trifluoroacetate salt |
| P162 | 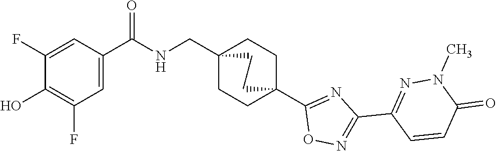 [SMILES: Cn1nc(-c2noc([C@]34CC[C@](CNC(=O)c5cc(F)c(O)c(F)c5)(CC3)CC4)n2)ccc1=O] | 3,5-difluoro-4-hydroxy-N-({4-[3- (1-methyl-6-oxo-1,6- dihydropyridazin-3-yl)-1,2,4- oxadiazol-5- yl]bicyclo[2.2.2]octan-1- yl}methyl)benzamide |
| P163 | 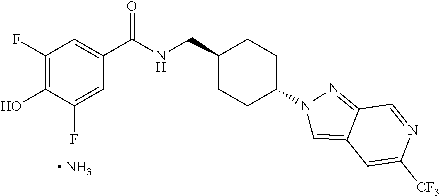 | 3,5-difluoro-4-hydroxy-N- ({(1r,4r)-4-[5-(trifluoromethyl)- 2H-pyrazolo[3,4-c]pyridin-2- yl]cyclohexyl}methyl)benzamide, ammonium salt |
| P164 | 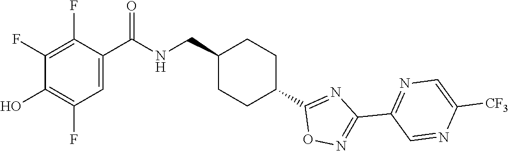 [SMILES: O=C(NC[C@H]1CC[C@H](c2nc(-c3cnc(C(F)(F)F)cn3)no2)CC1)c1cc(F)c(O)c(F)c1F] | 2,3,5-trifluoro-4-hydroxy-N- {[(1r,4r)-4-{3-[5- (trifluoromethyl)pyrazin-2-yl]- 1,2,4-oxadiazol-5- yl}cyclohexyl]methyl}benzamide |
| P165 | 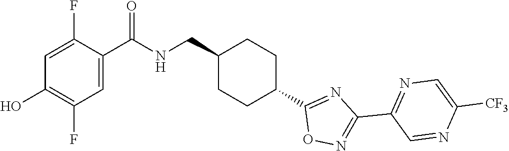 [SMILES: O=C(NC[C@H]1CC[C@H](c2nc(-c3cnc(C(F)(F)F)cn3)no2)CC1)c1cc(F)c(O)cc1F] | 2,5-difluoro-4-hydroxy-N- {[(1r,4r)-4-{3-[5- (trifluoromethyl)pyrazin-2-yl]- 1,2,4-oxadiazol-5- yl}cyclohexyl]methyl}benzamide |
| P166 | 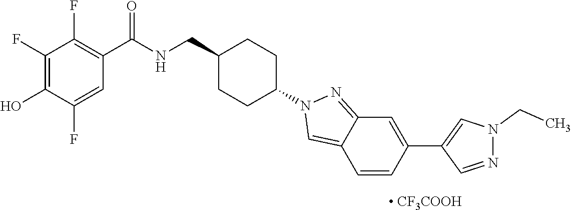 [SMILES: CCn1cc(-c2ccc3cn([C@H]4CC[C@H](CNC(=O)c5cc(F)c(O)c(F)c5F)CC4)nc3c2)cn1 ; "· CF3COOH"] | N-({(1r,4r)-4-[6-(1-ethyl-1H- pyrazol-4-yl)-2H-indazol-2- yl]cyclohexyl}methyl)-2,3,5- trifluoro-4-hydroxybenzamide, trifluoroacetate salt |
| P167 | 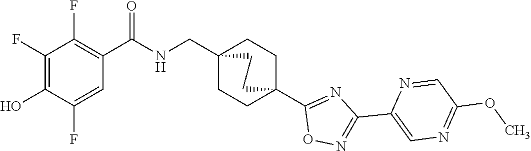 [SMILES: COc1cnc(-c2noc([C@]34CC[C@](CNC(=O)c5cc(F)c(O)c(F)c5F)(CC3)CC4)n2)cn1] | 2,3,5-trifluoro-4-hydroxy-N-({4- [3-(5-methoxypyrazin-2-yl)- 1,2,4-oxadiazol-5- yl]bicyclo[2.2.2]octan-1- yl}methyl)benzamide |
| P168 | 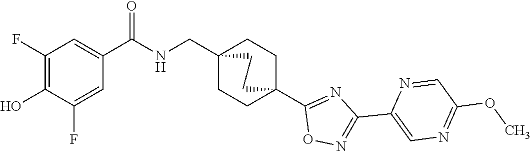 [SMILES: COc1cnc(-c2noc([C@]34CC[C@](CNC(=O)c5cc(F)c(O)c(F)c5)(CC3)CC4)n2)cn1] | 3,5-difluoro-4-hydroxy-N-({4-[3- (5-methoxypyrazin-2-yl)-1,2,4- oxadiazol-5- yl]bicyclo[2.2.2]octan-1- yl}methyl)benzamide |
| P169 | 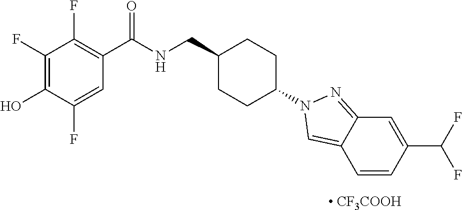 [SMILES: O=C(NC[C@H]1CC[C@H](n2cc3ccc(C(F)F)cc3n2)CC1)c1cc(F)c(O)c(F)c1F ; "· CF3COOH"] | N-({(1r,4r)-4-[6-(difluoromethyl)- 2H-indazol-2- yl]cyclohexyl}methyl)-2,3,5- trifluoro-4-hydroxybenzamide, trifluoroacetate salt |
| P170 | 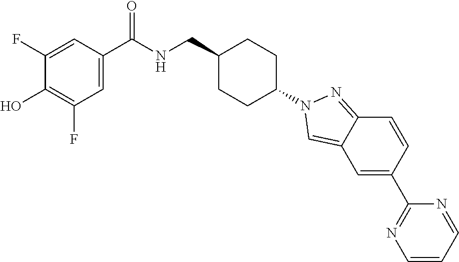 [SMILES: O=C(NC[C@H]1CC[C@H](n2cc3cc(-c4ncccn4)ccc3n2)CC1)c1cc(F)c(O)c(F)c1] | 3,5-difluoro-4-hydroxy-N- ({(1r,4r)-4-[5-(pyrimidin-2-yl)- 2H-indazol-2- yl]cyclohexyl}methyl)benzamide |
| P171 | 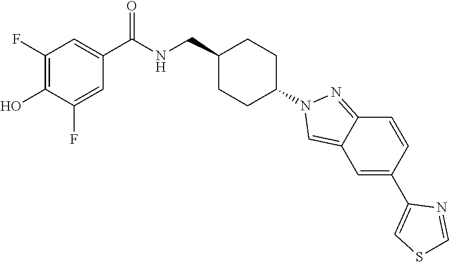 [SMILES: O=C(NC[C@H]1CC[C@H](n2cc3cc(-c4cscn4)ccc3n2)CC1)c1cc(F)c(O)c(F)c1] | 3,5-difluoro-4-hydroxy-N- ({(1r,4r)-4-[5-(1,3-thiazol-4-yl)- 2H-indazol-2- yl]cyclohexyl}methyl)benzamide |
| P172 | 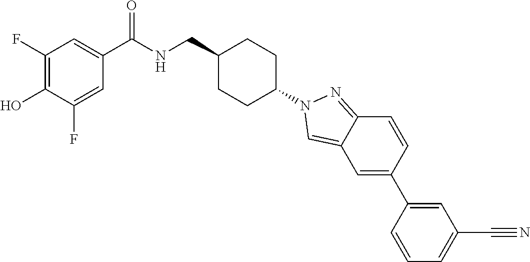 [SMILES: N#Cc1cccc(-c2ccc3nn([C@H]4CC[C@H](CNC(=O)c5cc(F)c(O)c(F)c5)CC4)cc3c2)c1] | N-({(1r,4r)-4-[5-(3- cyanophenyl)-2H-indazol-2- yl]cyclohexyl}methyl)-3,5- difluoro-4-hydroxybenzamide |
| P173 | 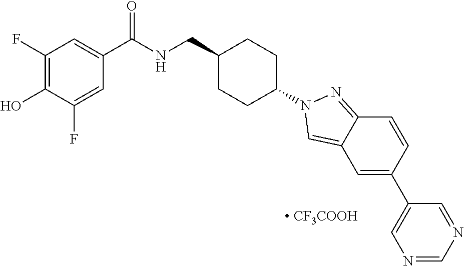 [SMILES: O=C(NC[C@H]1CC[C@H](n2cc3cc(-c4cncnc4)ccc3n2)CC1)c1cc(F)c(O)c(F)c1 ; "· CF3COOH"] | 3,5-difluoro-4-hydroxy-N- ({(1r,4r)-4-[5-(pyrimidin-5-yl)- 2H-indazol-2- yl]cyclohexyl}methyl)benzamide, trifluoroacetate salt |
| P174 | 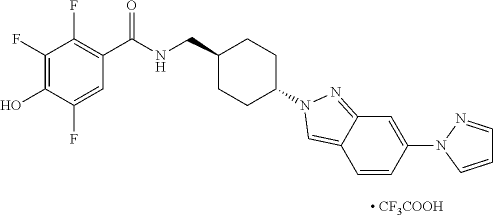 [SMILES: O=C(NC[C@H]1CC[C@H](n2cc3ccc(-n4cccn4)cc3n2)CC1)c1cc(F)c(O)c(F)c1F ; "· CF3COOH"] | 2,3,5-trifluoro-4-hydroxy-N- ({(1r,4r)-4-[6-(1H-pyrazol-1-yl)- 2H-indazol-2- yl]cyclohexyl}methyl)benzamide, trifluoroacetate salt |
| P175 | 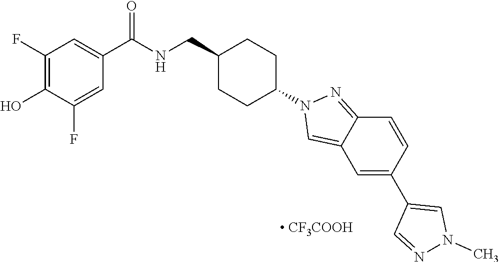 [SMILES: Cn1cc(-c2ccc3nn([C@H]4CC[C@H](CNC(=O)c5cc(F)c(O)c(F)c5)CC4)cc3c2)cn1 ; "· CF3COOH"] | 3,5-difluoro-4-hydroxy-N- ({(1r,4r)-4-[5-(1-methyl-1H- pyrazol-4-yl)-2H-indazol-2- yl]cyclohexyl}methyl)benzamide, trifluoroacetate salt |
| P176 | 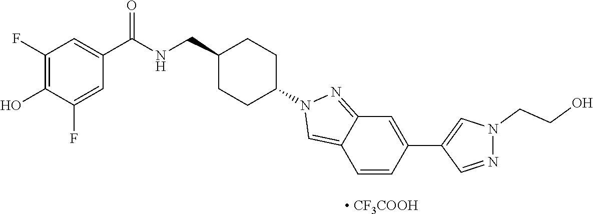 [SMILES: O=C(NC[C@H]1CC[C@H](n2cc3ccc(-c4cnn(CCO)c4)cc3n2)CC1)c1cc(F)c(O)c(F)c1 ; "· CF3COOH"] | 3,5-difluoro-4-hydroxy-N- {[(1r,4r)-4-{6-[1-(2- hydroxyethyl)-1H-pyrazol-4-yl]- 2H-indazol-2- yl}cyclohexyl]methyl}benzamide, trifluoroacetate salt |
| P177 | 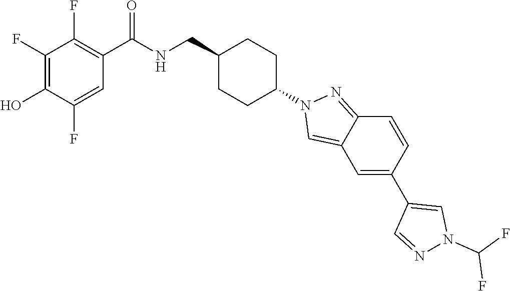 [SMILES: O=C(NC[C@H]1CC[C@H](n2cc3cc(-c4cnn(C(F)F)c4)ccc3n2)CC1)c1cc(F)c(O)c(F)c1F] | N-{[(1r,4r)-4-{5-[1- (difluoromethyl)-1H-pyrazol-4- yl]-2H-indazol-2- yl}cyclohexyl]methyl}-2,3,5- trifluoro-4-hydroxybenzamide |
| P178 | 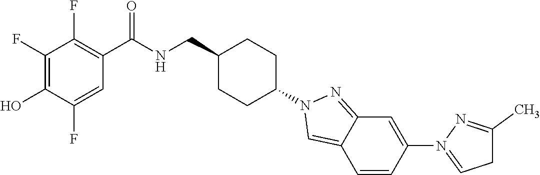 | 2,3,5-trifluoro-4-hydroxy-N- ({(1r,4r)-4-[6-(3-methyl-1H- pyrazol-1-yl)-2H-indazol-2- yl]cyclohexyl}methyl)benzamide |
| P179 | 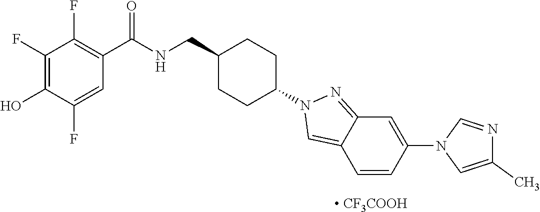 [SMILES: Cc1cn(-c2ccc3cn([C@H]4CC[C@H](CNC(=O)c5cc(F)c(O)c(F)c5F)CC4)nc3c2)cn1 ; "· CF3COOH"] | 2,3,5-trifluoro-4-hydroxy-N- ({(1r,4r)-4-[6-(4-methyl-1H- imidazol-1-yl)-2H-indazol-2- yl]cyclohexyl}methyl)benzamide |
| P180 | 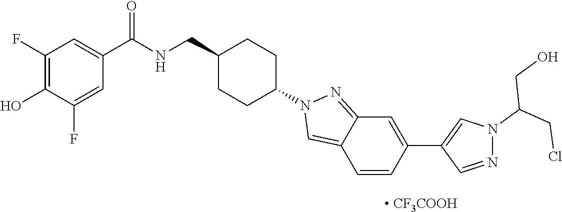 [SMILES: O=C(NC[C@H]1CC[C@H](n2cc3ccc(-c4cnn(C(CO)CCl)c4)cc3n2)CC1)c1cc(F)c(O)c(F)c1 ; "· CF3COOH"] | N-{[(1r,4r)-4-{6-[1-(1-chloro-3- hydroxypropan-2-yl)-1H- pyrazol-4-yl]-2H-indazol-2- yl}cyclohexyl]methyl}-3,5- difluoro-4-hydroxybenzamide, trifluoroacetate salt |
| P181 | 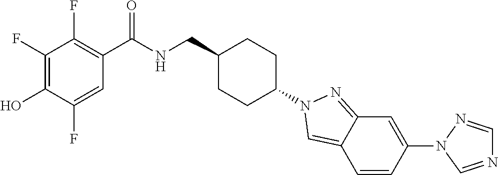 [SMILES: O=C(NC[C@H]1CC[C@H](n2cc3ccc(-n4cncn4)cc3n2)CC1)c1cc(F)c(O)c(F)c1F] | 2,3,5-trifluoro-4-hydroxy-N- ({(1r,4r)-4-[6-(1H-1,2,4-triazol- 1-yl)-2H-indazol-2- yl]cyclohexyl}methyl)benzamide |
| P182 | 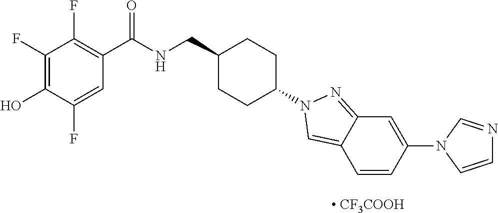 [SMILES: O=C(NC[C@H]1CC[C@H](n2cc3ccc(-n4ccnc4)cc3n2)CC1)c1cc(F)c(O)c(F)c1F ; "· CF3COOH"] | 2,3,5-trifluoro-4-hydroxy-N- ({(1r,4r)-4-[6-(1H-imidazol-1-yl)- 2H-indazol-2- yl]cyclohexyl}methyl)benzamide, trifluoroacetate salt |
| P183 | 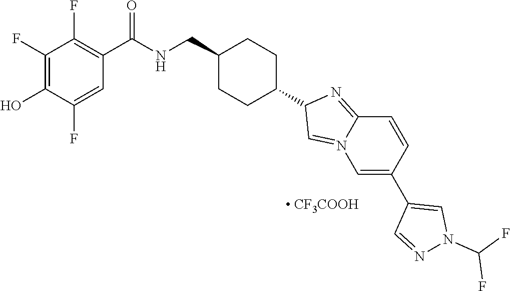 | N-{[(1r,4r)-4-{6-[1- (difluoromethyl)-1H-pyrazol-4- yl]imidazo[1,2-a]pyridin-2- yl}cyclohexyl]methyl}-2,3,5- trifluoro-4-hydroxybenzamide, trifluoroacetate salt |
| P184 | 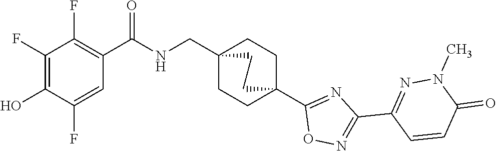 [SMILES: Cn1nc(-c2noc([C@]34CC[C@](CNC(=O)c5cc(F)c(O)c(F)c5F)(CC3)CC4)n2)ccc1=O] | 2,3,5-trifluoro-4-hydroxy-N-({4- [3-(1-methyl-6-oxo-1,6- dihydropyridazin-3-yl)-1,2,4- oxadiazol-5- yl]bicyclo[2.2.2]octan-1- yl}methyl)benzamide |
| P185 | 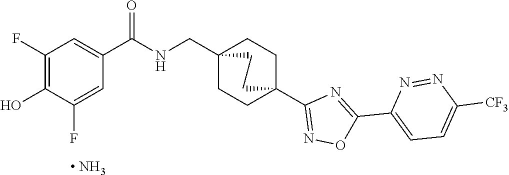 | 3,5-difluoro-4-hydroxy-N-[(4-{5- [6-(trifluoromethyl)pyridazin-3- yl]-1,2,4-oxadiazol-3- yl}bicyclo[2.2.2]octan-1- yl)methyl]benzamide, ammonium salt |
| P186 | 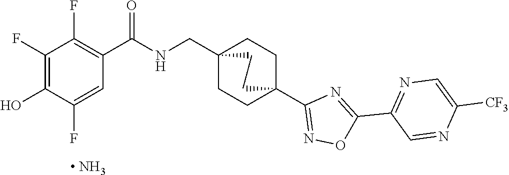 | 2,3,5-trifluoro-4-hydroxy-N-[(4- {5-[5-(trifluoromethyl)pyrazin-2- yl]-1,2,4-oxadiazol-3- yl}bicyclo[2.2.2]octan-1- yl)methyl]benzamide, ammonium salt |
| P187 | 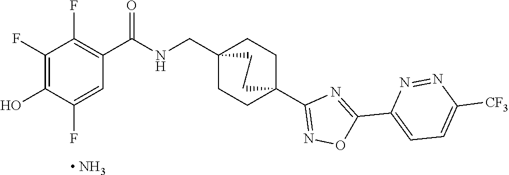 | 2,3,5-trifluoro-4-hydroxy-N-({4- [5-(6-methylpyridazin-3-yl)- 1,2,4-oxadiazol-3- yl]bicyclo[2.2.2]octan-1- yl}methyl)benzamide, ammonium salt |
| P188 | 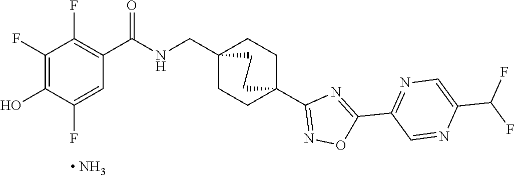 | N-[(4-{5-[5- (difluoromethyl)pyrazin-2-yl]- 1,2,4-oxadiazol-3- yl}bicyclo[2.2.2]octan-1- yl)methyl]-2,3,5-trifluoro-4- hydroxybenzamide, ammonium salt |
| P189 | 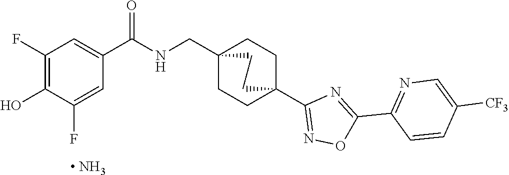 | 3,5-difluoro-4-hydroxy-N-[(4-{5- [5-(trifluoromethyl)pyridin-2-yl]- 1,2,4-oxadiazol-3- yl}bicyclo[2.2.2]octan-1- yl)methyl]benzamide, ammonium salt |
| P190 | 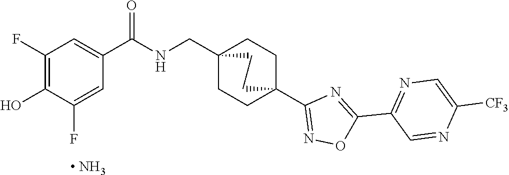 | 3,5-difluoro-4-hydroxy-N-[(4-{5- [5-(trifluoromethyl)pyrazin-2-yl]- 1,2,4-oxadiazol-3- yl}bicyclo[2.2.2]octan-1- yl)methyl]benzamide, ammonium salt |
| P191 | 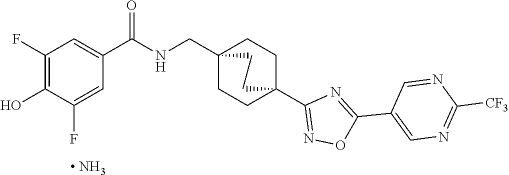 | 3,5-difluoro-4-hydroxy-N-[(4-{5- [2-(trifluoromethyl)pyrimidin-5- yl]-1,2,4-oxadiazol-3- yl}bicyclo[2.2.2]octan-1- yl)methyl]benzamide, ammonium salt |
| P192 | 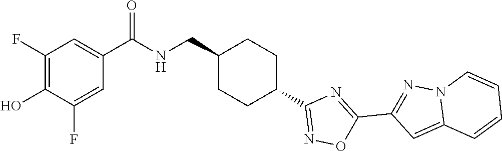 [SMILES: O=C(NC[C@H]1CC[C@H](c2noc(-c3cc4ccccn4n3)n2)CC1)c1cc(F)c(O)c(F)c1] | 3,5-difluoro-4-hydroxy-N- ({(1r,4r)-4-[5-(pyrazolo[1,5- a]pyridin-2-yl)-1,2,4-oxadiazol- 3- yl]cyclohexyl}methyl)benzamide |
| P193 | 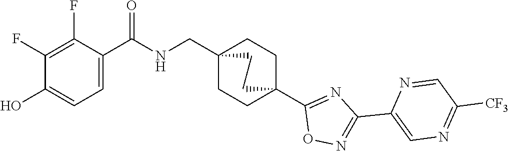 [SMILES: O=C(NC[C@]12CC[C@](c3nc(-c4cnc(C(F)(F)F)cn4)no3)(CC1)CC2)c1ccc(O)c(F)c1F] | 2,3-difluoro-4-hydroxy-N-[(4-{3- [5-(trifluoromethyl)pyrazin-2-yl]- 1,2,4-oxadiazol-5- yl}bicyclo[2.2.2]octan-1- yl)methyl]benzamide |
| P194 | 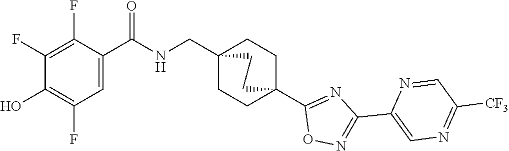 [SMILES: O=C(NC[C@]12CC[C@](c3nc(-c4cnc(C(F)(F)F)cn4)no3)(CC1)CC2)c1cc(F)c(O)c(F)c1F] | 2,3,5-trifluoro-4-hydroxy-N-[(4- {3-[5-(trifluoromethyl)pyrazin-2- yl]-1,2,4-oxadiazol-5- yl}bicyclo[2.2.2]octan-1- yl)methyl]benzamide |
| P195 | 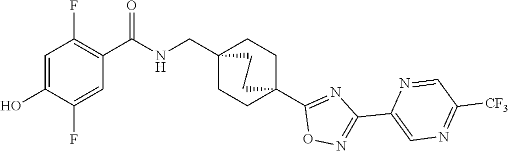 [SMILES: O=C(NC[C@]12CC[C@](c3nc(-c4cnc(C(F)(F)F)cn4)no3)(CC1)CC2)c1cc(F)c(O)cc1F] | 2,5-difluoro-4-hydroxy-N-[(4-{3- [5-(trifluoromethyl)pyrazin-2-yl]- 1,2,4-oxadiazol-5- yl}bicyclo[2.2.2]octan-1- yl)methyl]benzamide |
| P196 | 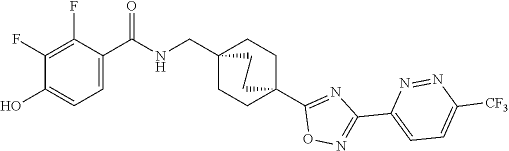 [SMILES: O=C(NC[C@]12CC[C@](c3nc(-c4ccc(C(F)(F)F)nn4)no3)(CC1)CC2)c1ccc(O)c(F)c1F] | 2,3-difluoro-4-hydroxy-N-[(4-{3- [6-(trifluoromethyl)pyridazin-3- yl]-1,2,4-oxadiazol-5- yl}bicyclo[2.2.2]octan-1- yl)methyl]benzamide |
| P197 | 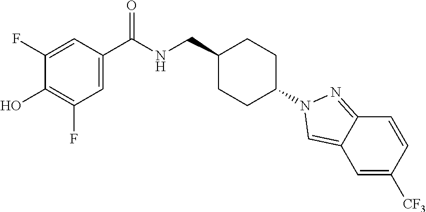 [SMILES: O=C(NC[C@H]1CC[C@H](n2cc3cc(C(F)(F)F)ccc3n2)CC1)c1cc(F)c(O)c(F)c1] | 3,5-difluoro-4-hydroxy-N- ({(1r,4r)-4-[5-(trifluoromethyl)- 2H-indazol-2- yl]cyclohexyl}methyl)benzamide |
| P198 | 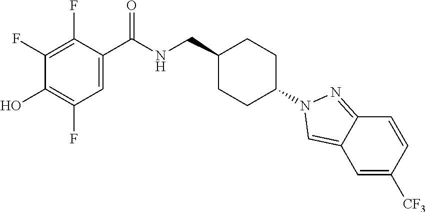 [SMILES: O=C(NC[C@H]1CC[C@H](n2cc3cc(C(F)(F)F)ccc3n2)CC1)c1cc(F)c(O)c(F)c1F] | 2,3,5-trifluoro-4-hydroxy-N- ({(1r,4r)-4-[5-(trifluoromethyl)- 2H-indazol-2- yl]cyclohexyl}methyl)benzamide |
| P199 | 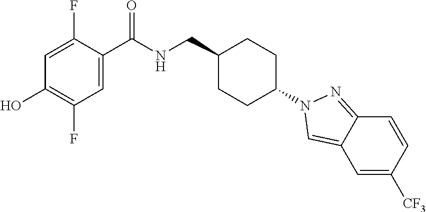 [SMILES: O=C(NC[C@H]1CC[C@H](n2cc3cc(C(F)(F)F)ccc3n2)CC1)c1cc(F)c(O)cc1F] | 2,5-difluoro-4-hydroxy-N- ({(1r,4r)-4-[5-(trifluoromethyl)- 2H-indazol-2- yl]cyclohexyl}methyl)benzamide |
| P200 | 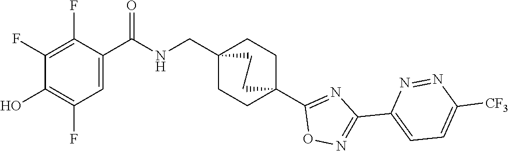 [SMILES: O=C(NC[C@]12CC[C@](c3nc(-c4ccc(C(F)(F)F)nn4)no3)(CC1)CC2)c1cc(F)c(O)c(F)c1F] | 2,3,5-trifluoro-4-hydroxy-N-[(4- {3-[6-(trifluoromethyl)pyridazin- 3-yl]-1,2,4-oxadiazol-5- yl}bicyclo[2.2.2]octan-1- yl)methyl]benzamide |
| P201 | 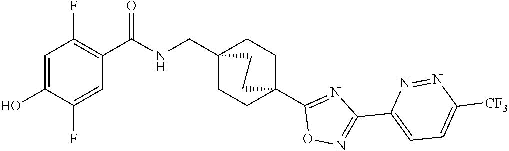 [SMILES: O=C(NC[C@]12CC[C@](c3nc(-c4ccc(C(F)(F)F)nn4)no3)(CC1)CC2)c1cc(F)c(O)cc1F] | 2,5-difluoro-4-hydroxy-N-[(4-{3- [6-(trifluoromethyl)pyridazin-3- yl]-1,2,4-oxadiazol-5- yl}bicyclo[2.2.2]octan-1- yl)methyl]benzamide |
| P202 | 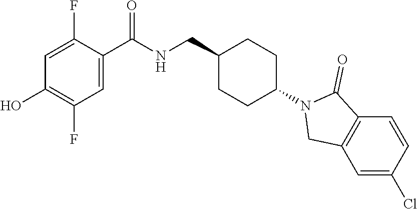 [SMILES: O=C(NC[C@H]1CC[C@H](N2Cc3cc(Cl)ccc3C2=O)CC1)c1cc(F)c(O)cc1F] | N-{[(1r,4r)-4-(5-chloro-1-oxo- 1,3-dihydro-2H-isoindol-2- yl)cyclohexyl]methyl}-2,5- difluoro-4-hydroxybenzamide |
| P203 | 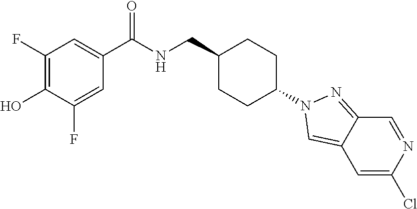 [SMILES: O=C(NC[C@H]1CC[C@H](n2cc3cc(Cl)ncc3n2)CC1)c1cc(F)c(O)c(F)c1] | N-{[(1r,4r)-4-(5-chloro-2H- pyrazolo[3,4-c]pyridin-2- yl)cyclohexyl]methyl}-3,5- difluoro-4-hydroxybenzamide |
| P204 | 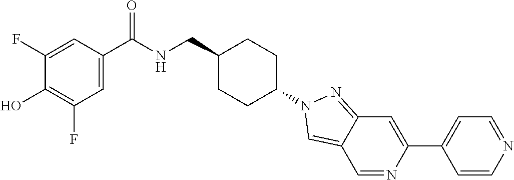 [SMILES: O=C(NC[C@H]1CC[C@H](n2cc3cnc(-c4ccncc4)cc3n2)CC1)c1cc(F)c(O)c(F)c1] | 3,5-difluoro-4-hydroxy-N- ({(1r,4r)-4-[6-(pyridin-4-yl)-2H- pyrazolo[4,3-c]pyridin-2- yl]cyclohexyl}methyl)benzamide |
| P205 | 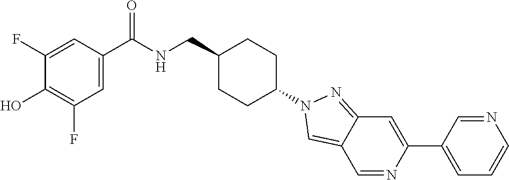 [SMILES: O=C(NC[C@H]1CC[C@H](n2cc3cnc(-c4cccnc4)cc3n2)CC1)c1cc(F)c(O)c(F)c1] | 3,5-difluoro-4-hydroxy-N- ({(1r,4r)-4-[6-(pyridin-3-yl)-2H- pyrazolo[4,3-c]pyridin-2- yl]cyclohexyl}methyl)benzamide |
| P206 | 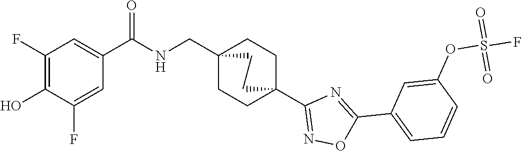 [SMILES: O=C(NC[C@]12CC[C@](c3noc(-c4cccc(OS(=O)(=O)F)c4)n3)(CC1)CC2)c1cc(F)c(O)c(F)c1] | 3-(3-{4-[(3,5-difluoro-4- hydroxybenzamido)methyl] bicyclo[2.2.2]octan-1-yl}-1,2,4- oxadiazol-5-yl)phenyl sulfurofluoridate |
| P207 | 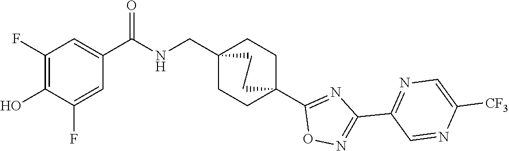 [SMILES: O=C(NC[C@]12CC[C@](c3nc(-c4cnc(C(F)(F)F)cn4)no3)(CC1)CC2)c1cc(F)c(O)c(F)c1] | 3,5-difluoro-4-hydroxy-N-[(4-{3- [5-(trifluoromethyl)pyrazin-2-yl]- 1,2,4-oxadiazol-5- yl}bicyclo[2.2.2]octan-1- yl)methyl]benzamide |
| P208 | 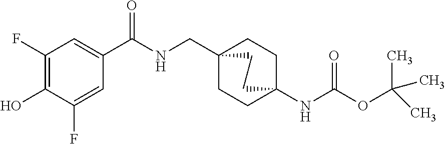 [SMILES: CC(C)(C)OC(=O)N[C@]12CC[C@](CNC(=O)c3cc(F)c(O)c(F)c3)(CC1)CC2] | tert-butyl {4-[(3,5-difluoro-4- hydroxybenzamido)methyl] bicyclo[2.2.2]octan-1-yl}carbamate |
| P209 | 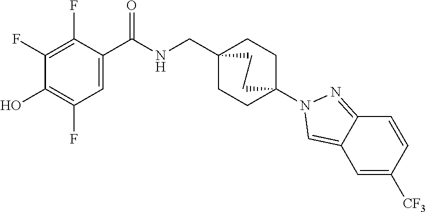 [SMILES: O=C(NC[C@]12CC[C@](n3cc4cc(C(F)(F)F)ccc4n3)(CC1)CC2)c1cc(F)c(O)c(F)c1F] | 2,3,5-trifluoro-4-hydroxy-N-({4- [5-(trifluoromethyl)-2H-indazol- 2-yl]bicyclo[2.2.2]octan-1- yl}methyl)benzamide |
| P210 | 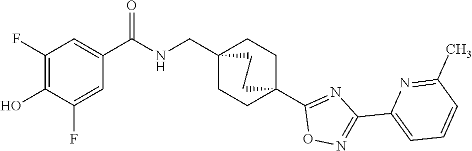 [SMILES: Cc1cccc(-c2noc([C@]34CC[C@](CNC(=O)c5cc(F)c(O)c(F)c5)(CC3)CC4)n2)n1] | 3,5-difluoro-4-hydroxy-N-({4-[3- (6-methylpyridin-2-yl)-1,2,4- oxadiazol-5- yl]bicyclo[2.2.2]octan-1- yl}methyl)benzamide |
| P211 | 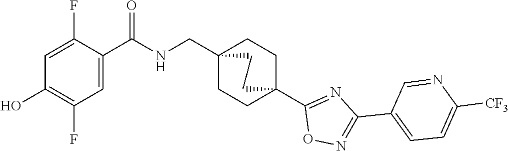 [SMILES: O=C(NC[C@]12CC[C@](c3nc(-c4ccc(C(F)(F)F)nc4)no3)(CC1)CC2)c1cc(F)c(O)cc1F] | 2,5-difluoro-4-hydroxy-N-[(4-{3- [6-(trifluoromethyl)pyridin-3-yl]- 1,2,4-oxadiazol-5- yl}bicyclo[2.2.2]octan-1- yl)methyl]benzamide |
| P212 | 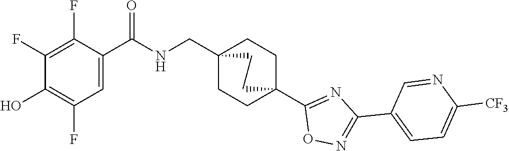 [SMILES: O=C(NC[C@]12CC[C@](c3nc(-c4ccc(C(F)(F)F)nc4)no3)(CC1)CC2)c1cc(F)c(O)c(F)c1F] | 2,3,5-trifluoro-4-hydroxy-N-[(4- {3-[6-(trifluoromethyl)pyridin-3- yl]-1,2,4-oxadiazol-5- yl}bicyclo[2.2.2]octan-1- yl)methyl]benzamide |
| P213 | 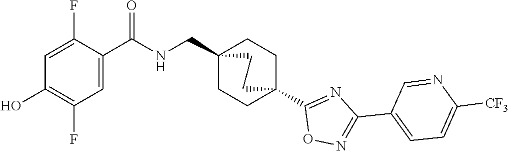 [SMILES: O=C(NC[C@]12CC[C@@](c3nc(-c4ccc(C(F)(F)F)nc4)no3)(CC1)CC2)c1cc(F)c(O)cc1F] | 2,5-difluoro-4-hydroxy-N- {[(1r,4r)-4-{3-[6- (trifluoromethyl)pyridin-3-yl]- 1,2,4-oxadiazol-5- yl}cyclohexyl]methyl}benzamide |
| P214 | 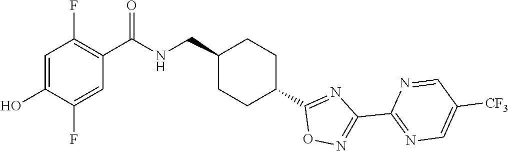 [SMILES: O=C(NC[C@H]1CC[C@H](c2nc(-c3ncc(C(F)(F)F)cn3)no2)CC1)c1cc(F)c(O)cc1F] | 2,5-difluoro-4-hydroxy-N- {[(1r,4r)-4-{3-[5- (trifluoromethyl)pyrimidin-2-yl]- 1,2,4-oxadiazol-5- yl}cyclohexyl]methyl}benzamide |
| P215 | 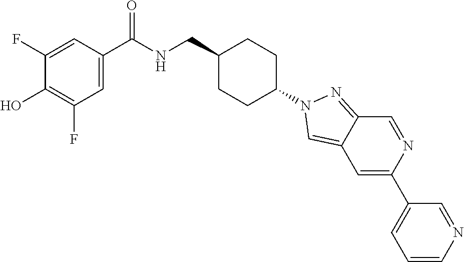 [SMILES: O=C(NC[C@H]1CC[C@H](n2cc3cc(-c4cccnc4)ncc3n2)CC1)c1cc(F)c(O)c(F)c1] | 3,5-difluoro-4-hydroxy-N- ({(1r,4r)-4-[5-(pyridin-3-yl)-2H- pyrazolo[3,4-c]pyridin-2- yl]cyclohexyl}methyl)benzamide |
| P216 | 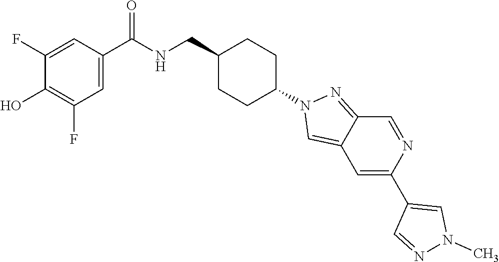 [SMILES: Cn1cc(-c2cc3cn([C@H]4CC[C@H](CNC(=O)c5cc(F)c(O)c(F)c5)CC4)nc3cn2)cn1] | 3,5-difluoro-4-hydroxy-N- ({(1r,4r)-4-[5-(1-methyl-1H- pyrazol-4-yl)-2H-pyrazolo[3,4- c]pyridin-2- yl]cyclohexyl}methyl)benzamide |
| P217 | 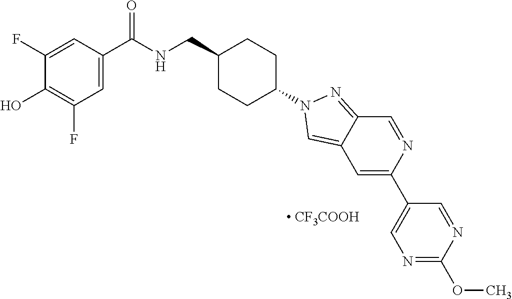 [SMILES: COc1ncc(-c2cc3cn([C@H]4CC[C@H](CNC(=O)c5cc(F)c(O)c(F)c5)CC4)nc3cn2)cn1 ; "· CF3COOH"] | 3,5-difluoro-4-hydroxy-N- ({(1r,4r)-4-[5-(2- methoxypyrimidin-5-yl)-2H- pyrazolo[3,4-c]pyridin-2- yl]cyclohexyl}methyl)benzamide, trifluoroacetate salt |
| P218 | 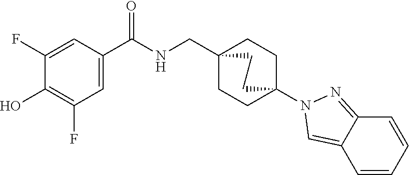 [SMILES: O=C(NC[C@]12CC[C@](n3cc4ccccc4n3)(CC1)CC2)c1cc(F)c(O)c(F)c1] | 3,5-difluoro-4-hydroxy-N-{[4- (2H-indazol-2- yl)bicyclo[2.2.2]octan-1- yl]methyl}benzamide |
| P219 | 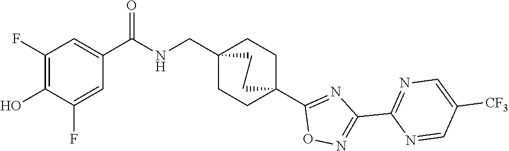 [SMILES: O=C(NC[C@]12CC[C@](c3nc(-c4ncc(C(F)(F)F)cn4)no3)(CC1)CC2)c1cc(F)c(O)c(F)c1] | 3,5-difluoro-4-hydroxy-N-[(4-{3- [5-(trifluoromethyl)pyrimidin-2- yl]-1,2,4-oxadiazol-5- yl}bicyclo[2.2.2]octan-1- yl)methyl]benzamide |
| P220 | 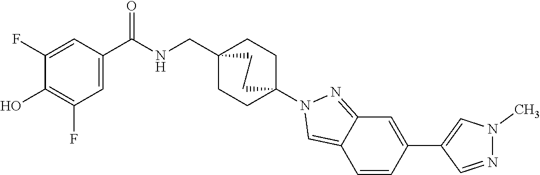 [SMILES: Cn1cc(-c2ccc3cn([C@]45CC[C@](CNC(=O)c6cc(F)c(O)c(F)c6)(CC4)CC5)nc3c2)cn1] | 3,5-difluoro-4-hydroxy-N-({4-[6- (1-methyl-1H-pyrazol-4-yl)-2H- indazol-2-yl]bicyclo[2.2.2]octan- 1-yl}methyl)benzamide |
| P221 | 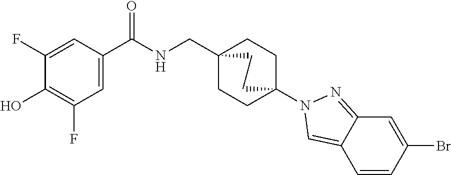 [SMILES: O=C(NC[C@]12CC[C@](n3cc4ccc(Br)cc4n3)(CC1)CC2)c1cc(F)c(O)c(F)c1] | N-{[4-(6-bromo-2H-indazol-2- yl)bicyclo[2.2.2]octan-1- yl]methyl}-3,5-difluoro-4- hydroxybenzamide |
| P222 | 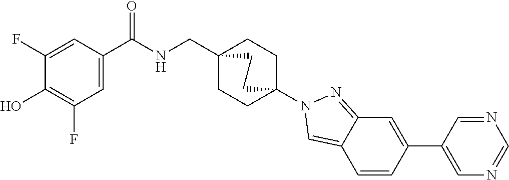 [SMILES: O=C(NC[C@]12CC[C@](n3cc4ccc(-c5cncnc5)cc4n3)(CC1)CC2)c1cc(F)c(O)c(F)c1] | 3,5-difluoro-4-hydroxy-N-({4-[6- (pyrimidin-5-yl)-2H-indazol-2- yl]bicyclo[2.2.2]octan-1- yl}methyl)benzamide |
| P223 | 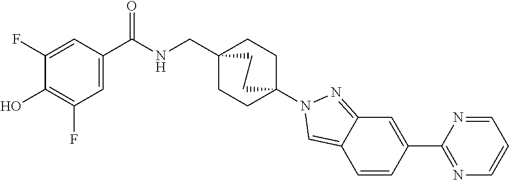 [SMILES: O=C(NC[C@]12CC[C@](n3cc4ccc(-c5ncccn5)cc4n3)(CC1)CC2)c1cc(F)c(O)c(F)c1] | 3,5-difluoro-4-hydroxy-N-({4-[6- (pyrimidin-2-yl)-2H-indazol-2- yl]bicyclo[2.2.2]octan-1- yl}methyl)benzamide |
| P224 | 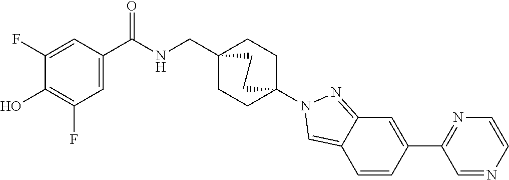 [SMILES: O=C(NC[C@]12CC[C@](n3cc4ccc(-c5cnccn5)cc4n3)(CC1)CC2)c1cc(F)c(O)c(F)c1] | 3,5-difluoro-4-hydroxy-N-({4-[6- (pyrazin-2-yl)-2H-indazol-2- yl]bicyclo[2.2.2]octan-1- yl}methyl)benzamide |
| P225 | 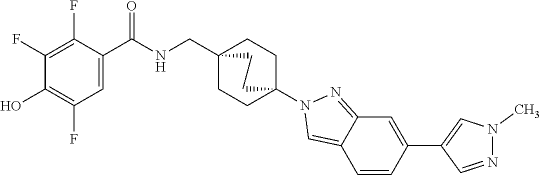 [SMILES: Cn1cc(-c2ccc3cn([C@]45CC[C@](CNC(=O)c6cc(F)c(O)c(F)c6F)(CC4)CC5)nc3c2)cn1] | 2,3,5-trifluoro-4-hydroxy-N-({4- [6-(1-methyl-1H-pyrazol-4-yl)- 2H-indazol-2- yl]bicyclo[2.2.2]octan-1- yl}methyl)benzamide |
| P226 | 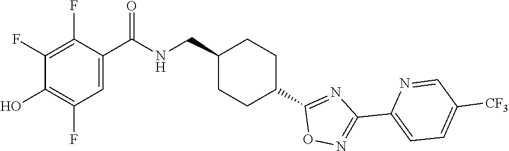 | 2,3,5-trifluoro-4-hydroxy-N- {[(1r,4r)-4-{3-[5- (trifluoromethyl)pyridin-2-yl]- 1,2,4-oxadiazol-5- yl}cyclohexyl]methyl}benzamide |

TABLE 2
Method of synthesis and physicochemical data for Preparations P38-P226.

| | | ¹H NMR (400 MHz, DMSO-d₆) d; Mass spectrum, |
|---|---|---|
| | Method of synthesis; | observed ion m/z [M + H]⁺ or HPLC retention time; |
| | Non-commercial | Mass spectrum m/z [M + H]⁺ (unless otherwise |
| Preparation | starting materials | indicated) |
| P38 | P3¹ | 10.87 (br s, 1H), 8.47 (br t, J = 6 Hz, 1H), 8.38 (s, 1H), |
| | | 7.67 (d, J = 8.4 Hz, 1H), 7.64-7.53 (m, 3H), 7.20 (dd, J = |
| | | 8.7, 6.5 Hz, 1H), 7.00 (dd, J = 8.4, 6.5 Hz, 1H), 4.53- |
| | | 4.40 (m, 1H), 3.17 (dd, J = 6, 6 Hz, 2H), 2.20-2.09 (m, |
| | | 2H), 1.98-1.82 (m, 4H), 1.74-1.60 (m, 1H), 1.29-1.12 |
| | | (m, 2H); 386.2 |
| P39 | Preparation P20^{2,3}; P1 | 10.84 (br s, 1H), 8.46 (br t, J = 5.6 Hz, 1H), 8.24 (s, 1H), |
| | | 7.64-7.54 (m, 2H), 7.53 (d, J = 9.0 Hz, 1H), 6.91 (br s, |
| | | 1H), 6.67 (dd, J = 9.0, 2.2 Hz, 1H), 4.43-4.31 (m, 1H), |
| | | 3.77 (s, 3H), 3.17 (dd, J = 6, 6 Hz, 2H), 2.18-2.07 (m, |
| | | 2H), 1.95-1.78 (m, 4H), 1.72-1.59 (m, 1H), 1.27-1.12 |
| | | (m, 2H); 416.3 |
| P40 | Preparation P23^{4,2}; P14 | 10.84 (br s, 1H), 8.91 (d, J = 4.8 Hz, 2H), 8.67 (br s, 1H), |
| | | 8.51-8.44 (m, 2H), 8.09 (dd, J = 8.8, 1.4 Hz, 1H), 7.80 |
| | | (br d, J = 8.8 Hz, 1H), 7.64-7.54 (m, 2H), 7.43 (t, J = 4.8 |
| | | Hz, 1H), 4.58-4.47 (m, 1H), 3.18 (dd, J = 6, 6 Hz, 2H), |
| | | 2.24-2.14 (m, 2H), 2.00-1.86 (m, 4H), 1.76-1.63 (m, |
| | | 1H), 1.31-1.16 (m, 2H); 464.4 |
| P41 | Preparation P21⁵ | 11.36 (s, 1H), 8.47 (s, 1H), 8.33 (br t, J = 6 Hz, 1H), 7.73 |
| | | (d, J = 8.8 Hz, 1H), 7.69-7.67 (m, 1H), 7.30 (ddd, J = |
| | | 11.1, 6.3, 2.3 Hz, 1H), 7.01 (dd, J = 8.8, 1.8 Hz, 1H), 4.53- |
| | | 4.41 (m, 1H), 3.17 (dd, J = 6, 6 Hz, 2H), 2.20-2.10 (m, |
| | | 2H), 1.96-1.82 (m, 4H), 1.73-1.59 (m, 1H), 1.29-1.18 |
| | | (m, 2H); 438.3 (chlorine isotope pattern observed) |
| P42 | Preparation P21⁵ | 11.36 (s, 1H), 8.40 (s, 1H), 8.36-8.29 (m, 1H), 7.77 (br |
| | | d, J = 2 Hz, 1H), 7.64 (d, J = 9.1 Hz, 1H), 7.30 (ddd, J = |
| | | 11.1, 6.3, 2.3 Hz, 1H), 7.19 (dd, J = 9.1, 2.1 Hz, 1H), 4.54- |
| | | 4.42 (m, 1H), 3.17 (dd, J = 6, 6 Hz, 2H), 2.20-2.10 (m, |
| | | 2H), 1.97-1.82 (m, 4H), 1.73-1.59 (m, 1H), 1.28-1.14 |
| | | (m, 2H); 438.3 (chlorine isotope pattern observed) |
| P43 | Preparation P21⁶; P12 | 11.37 (br s, 1H), 8.92 (d, J = 4.9 Hz, 2H), 8.68 (br s, 1H), |
| | | 8.48 (br s, 1H), 8.37-8.30 (m, 1H), 8.10 (dd, J = 8.8, 1.4 |
| | | Hz, 1H), 7.80 (d, J = 8.9 Hz, 1H), 7.43 (t, J = 4.8 Hz, 1H), |
| | | 7.30 (ddd, J = 11.1, 6.3, 2.3 Hz, 1H), 4.58-4.47 (m, 1H), |
| | | 3.18 (dd, J = 6, 6 Hz, 2H), 2.25-2.14 (m, 2H), 2.02- |
| | | 1.86 (m, 4H), 1.76-1.62 (m, 1H), 1.32-1.16 (m, 2H); 482.2 |
| P44 | Preparation P21⁷; P12 | 11.37 (br s, 1H), 9.34 (d, J = 1.6 Hz, 1H), 8.72 (dd, J = |
| | | 2.5, 1.5 Hz, 1H), 8.60 (d, J = 2.5 Hz, 1H), 8.48 (br s, 1H), |
| | | 8.44-8.41 (m, 1H), 8.37-8.30 (m, 1H), 7.83 (br s, 2H), |
| | | 7.30 (ddd, J = 11.1, 6.3, 2.3 Hz, 1H), 4.58-4.46 (m, 1H), |
| | | 3.18 (dd, J = 6, 6 Hz, 2H), 2.25-2.14 (m, 2H), 2.01- |
| | | 1.86 (m, 4H), 1.76-1.62 (m, 1H), 1.32-1.16 (m, 2H); 482.3 |
| P45 | Preparation P21⁸; C19 | 11.49 (v br s, 1H), 8.71 (s, 1H), 8.30-8.24 (m, 1H), 8.12 |
| | | (s, 1H), 7.84 (s, 1H), 7.60 (s, 1H), 7.44 (AB quartet, J_{AB} = |
| | | 9.2 Hz, Δv_{AB} = 34.5 Hz, 2H), 7.32-7.24 (m, 1H), 3.87 (s, |
| | | 3H), 3.14 (dd, J = 6, 6 Hz, 2H), 2.67-2.58 (m, 1H), 2.12- |
| | | 2.04 (m, 2H), 1.90-1.82 (m, 2H), 1.64-1.53 (m, 1H), |
| | | 1.48-1.37 (m, 2H), 1.17-1.06 (m, 2H); 484.2 |
| P46 | Preparation P45; P11 | ¹H NMR (400 MHz, DMSO-d₆), characteristic peaks: δ |
| | | 11.38 (br s, 1H), 8.76 (d, J = 7.0 Hz, 1H), 8.56 (s, 1H), |
| | | 8.37-8.27 (m, 1H), 8.22 (s, 1H), 7.99 (s, 1H), 7.86 (s, |
| | | 1H), 7.71 (d, J = 7.2 Hz, 1H), 7.36-7.22 (m, 1H), 3.92 (s, |
| | | 3H), 3.24-3.08 (m, 2H), 2.90-2.76 (m, 1H), 1.96-1.82 |
| | | (m, 2H), 1.70-1.39 (m, 3H), 1.24-1.05 (m, 2H); 484.3 |
| P47 | Preparation P45; P11 | 8.85 (s, 1H), 8.47 (d, J = 7.0 Hz, 1H), 8.41 (s, 1H), 8.30- |
| | | 8.22 (m, 1H), 8.03-7.68 (m, 2H), 7.63 (s, 1H), 7.28 (ddd, |
| | | J = 11.1, 6.3, 2.3 Hz, 1H), 7.20 (dd, J = 7.1, 1.8 Hz, 1H), |
| | | 3.15 (dd, J = 6, 6 Hz, 2H), 2.63 (tt, J = 11.9, 3.6 Hz, 1H), |
| | | 2.15-2.04 (m, 2H), 1.92-1.81 (m, 2H), 1.65-1.51 (m, |
| | | 1H), 1.50-1.34 (m, 2H), 1.20-1.04 (m, 2H); 520.3 |
| P48 | P1^{9,10} | 2.33 minutes¹¹; 457 |
| P49 | P1^{9,12} | 2.95 minutes¹¹; 562 |
| P50 | Preparation P28; P14 | 2.72 minutes¹¹; 516 |
| P51 | Preparation P28; P14 | 2.68 minutes¹¹; 506 |
| P52 | Preparation P28; P14 | 2.87 minutes¹¹; 508 |
| P53 | Preparation P28; P14 | 2.46 minutes¹¹; 494 |
| P54 | Preparation P28; P14 | 2.73 minutes¹³; 522 |
| P55 | Preparation P28; P14 | 2.52 minutes¹¹; 480 |
| P56 | Preparation P28; P14 | 2.72 minutes¹¹; 494 |
| P57 | Preparation P28; P14 | 2.43 minutes¹¹; 507 |
| P58 | Preparation P28; P14 | 2.80 minutes¹³; 496 |
| P59 | Preparation P28; P14 | 2.66 minutes¹¹; 492 |
| P60 | Preparation P28; P14 | 2.77 minutes¹¹; 502 |
| P61 | Preparation P28; P14 | 2.67 minutes¹³, 508 |
| P62 | Preparation P28; P14 | 2.96 minutes¹¹; 534 |
| P63 | Preparation P28; P14 | 2.59 minutes¹¹; 480 |
| P64 | Preparation P28; P14 | 2.55 minutes¹¹; 492 |
| P65 | Preparation P28; P14 | 2.89 minutes¹¹; 534 |
| P66 | Preparation P28; P14 | 2.50 minutes¹¹; 480 |
| P67 | Preparation P28; P14 | 2.68 minutes¹¹; 484 |
| P68 | Preparation P28; P14 | 2.73 minutes¹¹; 492 |
| P69 | Preparation P28; P14 | 2.68 minutes¹¹; 478 |
| P70 | Preparation P28; P14 | 2.92 minutes¹¹; 504 |
| P71 | Preparation P28; P14 | 2.99 minutes¹¹; 487 |
| P72 | Preparation P28; P14 | 2.96 minutes¹¹; 487 |
| P73 | Preparation P28; P14 | 2.20 minutes¹¹; 477 |
| P74 | Preparation P28; P14 | 2.57 minutes¹³; 494 |
| P75 | Preparation P28; P14 | 2.17 minutes¹¹; 521 |
| P76 | Preparation P21; P13 | 10.83 (s, 1H), 8.47 (br t, J = 5.8 Hz, 1H), 8.32 (br s, 1H), |
| | | 8.15 (s, 1H), 7.90 (br s, 1H), 7.75 (br s, 1H), 7.65 (d, J = |
| | | 8.7 Hz, 1H), 7.64-7.54 (m, 2H), 7.25 (dd, J = 8.7, 1.4 Hz, |
| | | 1H), 4.49-4.38 (m, 1H), 3.87 (s, 3H), 3.18 (dd, J = 6, 6 |
| | | Hz, 2H), 2.21-2.09 (m, 2H), 1.97-1.81 (m, 4H), 1.74- |
| | | 1.60 (m, 1H), 1.29-1.13 (m, 2H); 466.2 |
| P77 | Preparation P28; P14 | 2.10 minutes¹¹; 463 |
| P78 | Preparation P28; P14 | 2.56 minutes¹¹; 466 |
| P79 | Preparation P28; P14 | 2.47 minutes¹³; 478 |
| P80 | P14¹⁴ | 2.95 minutes¹⁵; 492.3 |
| P81 | Preparation P28; P14 | 2.74 minutes¹³, 464 |
| P82 | Preparation P28; P14 | 2.72 minutes¹¹; 494 |
| P83 | Preparation P28; P14 | 3.06 minutes¹¹; 492 |
| P84 | Preparation P28; P14 | 3.13 minutes¹¹; 462 |
| P85 | Preparation P28; P14 | 2.66 minutes¹¹; 469 |
| P86 | P14¹⁴ | 2.88 minutes¹⁵, 487.3 |
| P87 | Preparation P28; P14 | 2.50 minutes¹³; 464 |
| P88 | Preparation P28; P14 | 2.10 minutes¹¹; 463 |
| P89 | Preparation P28; P14 | 2.62 minutes¹¹; 494 |
| P90 | Preparation P28; P14 | 2.70 minutes¹¹; 478 |
| P91 | Preparation P28; P14 | 2.13 minutes¹¹; 463 |
| P92 | Preparation P28; P14 | 2.63 minutes¹³; 464 |
| P93 | Preparation P28; P14 | 2.44 minutes¹¹; 478 |
| P94 | Preparation P28¹⁶; P14 | 3.22 minutes¹¹; 532 |
| P95 | P14¹⁷ | 2.68 minutes¹³; 467 |
| P96 | P14¹⁷ | 2.86 minutes¹¹; 502 |
| P97 | Preparation P28¹⁶; P14 | 2.27 minutes¹¹; 477 |
| P98 | P14¹⁷ | 3.03 minutes¹³; 470 |
| P99 | P14¹⁷ | 2.90 minutes¹¹; 469 |
| P100 | P14¹⁷ | 2.30 minutes¹¹; 508 |
| P101 | P14¹⁷ | 3.15 minutes¹¹; 509 |
| P102 | P14¹⁷ | 2.67 minutes¹¹; 497 |
| P103 | Preparation P28¹⁶; P14 | 2.58 minutes¹¹; 478 |
| P104 | P14¹⁷ | 2.75 minutes¹¹; 483 |
| P105 | Preparation P28¹⁶; P14 | 2.91 minutes¹¹; 502 |
| P106 | Preparation P28¹⁶; P14 | 2.30 minutes¹¹; 477 |
| P107 | P14¹⁷ | 3.09 minutes¹¹; 519 |
| P108 | P14¹⁷ | 2.71 minutes¹¹; 467 |
| P109 | Preparation P28¹⁶; P14 | 2.88 minutes¹¹; 504 |
| P110 | P14¹⁷ | 2.86 minutes¹¹; 483 |
| P111 | Preparation P28¹⁶; P14 | 2.81 minutes¹¹; 494 |
| P112 | P14¹⁷ | 2.72 minutes¹¹; 469 |
| P113 | Preparation P28¹⁸; P14 | 2.29 minutes¹¹; 503 |
| P114 | P14¹⁷ | 2.91 minutes¹¹; 469 |
| P115 | Preparation P28¹⁶; P14 | 2.45 minutes¹¹; 493 |
| P116 | P14¹⁷ | 2.83 minutes¹¹; 469 |
| P117 | Preparation P28^{16,18}; P14 | 2.89 minutes¹³, 506 |
| P118 | P14¹⁷ | 2.61 minutes¹³; 484 |
| P119 | P14¹⁷ | 2.29 minutes¹¹; 502 |
| P120 | Preparation P28¹⁶; P14 | 2.69 minutes¹³; 493 |
| P121 | P14¹⁷ | 3.17 minutes¹¹; 509 |
| P122 | Preparation P28¹⁶; P14 | 2.77 minutes¹¹; 493 |
| P123 | P14¹⁷ | 2.91 minutes¹¹; 484 |
| P124 | Preparation P28¹⁶; P14 | 2.92 minutes¹¹; 493 |
| P125 | Preparation P28¹⁶; P14 | 2.78 minutes¹¹; 494 |
| P126 | P14¹⁷ | 2.83 minutes¹¹; 469 |
| P127 | P14¹⁷ | 2.94 minutes¹³; 453 |
| P128 | Preparation P28^{19,16}; | 2.52 minutes¹¹; 502 |
| | P14 | |
| P129 | Preparation P28¹⁶; P14 | 2.63 minutes¹¹; 503 |
| P130 | P14¹⁷ | 2.87 minutes¹¹; 554 |
| P131 | P14¹⁷ | 2.95 minutes¹¹; 483 |
| P132 | Preparation P28¹⁶; P14 | 3.15 minutes¹¹, 493 |
| P133 | Preparation P28¹⁶; P14 | 2.30 minutes¹¹; 502 |
| P134 | Preparation P28¹⁶; P14 | 2.26 minutes¹¹; 477 |
| P135 | Preparation P28¹⁶; P14 | 2.56 minutes¹¹; 503 |
| P136 | Preparation P28¹⁶; P14 | 2.38 minutes¹¹; 493 |
| P137 | P14¹⁷ | 2.99 minutes¹¹; 495 |
| P138 | Preparation P22; P3 | ¹H NMR (400 MHz, methanol-d₄), characteristic peaks: δ |
| | | 8.23 (s, 1H), 7.53-7.41 (m, 2H), 7.23-7.10 (m, 2H), |
| | | 6.39 (d, J = 7.2 Hz, 1H), 4.49-4.36 (m, 1H), 3.92 (s, 3H), |
| | | 2.31-2.18 (m, 2H), 2.09-1.91 (m, 4H), 1.87-1.72 (m, |
| | | 1H), 1.39-1.22 (m, 2H); 416.2 |
| P139 | Preparation P20²⁰ | 10.84 (s, 1H), 8.30 (br t, J = 6.3 Hz, 1H), 8.13 (d, J = 8.7 |
| | | Hz, 1H), 7.78 (d, J = 8.7 Hz, 1H), 7.65-7.55 (m, 2H), |
| | | 3.10 (d, J = 6.2 Hz, 2H), 2.72 (s, 3H), 2.04-1.93 (m, 6H), |
| | | 1.60-1.50 (m, 6H); 456.1 |
| P140 | Preparation P18^{21,22}; P1 | 10.82 (s, 1H), 8.29 (t, J = 6.3 Hz, 1H), 8.13 (d, J = 9.2 Hz, |
| | | 1H), 7.66-7.54 (m, 2H), 7.41 (d, J = 9.2 Hz, 1H), 4.12 (s, |
| | | 3H), 3.10 (d, J = 6.2 Hz, 2H), 2.05-1.90 (m, 6H), 1.62- |
| | | 1.48 (m, 6H); 472.6 |
| P141 | P1²³ | ¹H NMR (400 MHz, methanol-d₄) δ 8.50 (br s, 1H), 8.11- |
| | | 8.08 (m, 1H), 7.74 (br d, J = 9.1 Hz, 1H), 7.52-7.42 (m, |
| | | 2H), 7.44 (dd, J = 9.1, 1.8 Hz, 1H), 3.24 (s, 2H), 2.36- |
| | | 2.22 (m, 6H), 1.86-1.73 (m, 6H); 480.1 |
| P142 | P7²⁴ | ¹H NMR (400 MHz, methanol-d₄) δ 8.31 (br d, J = 8.9 Hz, |
| | | 2H), 7.70 (br d, J = 8.9 Hz, 2H), 7.52-7.41 (m, 2H), 3.19 |
| | | (s, 2H), 2.07-1.94 (m, 6H), 1.70-1.57 (m, 6H); 538.2 |
| P143 | Preparation P18²⁵; P4 | 2.67 minutes¹⁵; 446.4 |
| P144 | Preparation P18^{26,2}; P4 | 10.83 (s, 1H), 8.44 (br t, J = 5.8 Hz, 1H), 8.18 (AB quartet, |
| | | J_{AB} = 8.4 Hz, Δv_{AB} = 44.4 Hz, 4H), 7.64-7.52 (m, 2H), |
| | | 3.29 (s, 3H), 3.20-3.04 (m, 3H), 2.25-2.13 (m, 2H), |
| | | 1.93-1.81 (m, 2H), 1.69-1.50 (m, 3H), 1.22-1.07 (m, |
| | | 2H); 492.2 |
| P145 | Preparation P22²⁵; P3 | 2.48 minutes¹⁵; 416.5 |
| P146 | Preparation P22²⁵; P3 | 2.54 minutes¹⁵; 400.5 |
| P147 | Preparation P22²⁵; P3 | 2.61 minutes¹⁵; 400.5 |
| P148 | Preparation P32; P3 | 2.83 minutes¹⁵; 420.3 (chlorine isotope pattern observed) |
| P149 | Preparation P32; P3 | 2.63 minutes¹⁵; 404.4 |
| P150 | Preparation P32; P3 | 2.57 minutes¹⁵; 411.4 |
| P151 | Preparation P32; P3 | 2.83 minutes¹⁵; 420.3 (chlorine isotope pattern observed) |
| P152 | Preparation P32; P3 | 1.78 minutes¹⁵; 484.5 |
| P153 | Preparation P32; P3 | 2.94 minutes¹⁵; 454.4 |
| P154 | Preparation P29²⁵; P5 | 3.11 minutes¹⁵; 414.2 |
| P155 | Preparation P18²; P4 | 2.80 minutes¹⁵; 484.4 |
| P156 | Preparation P18^{21,2}; P4 | 3.30 minutes¹⁵, 509.6 |
| P157 | Preparation P20²⁷; P1 | 2.93 minutes¹⁵; 413.5 |
| P158 | Preparation P18^{21,2} | 3.04 minutes¹⁵; 491.4 |
| P159 | Preparation P20²⁸; P1 | 8.51 (AB quartet, J_{AB} = 8.8 Hz, Δv_{AB} = 27.5 Hz, 2H), 8.28 |
| | | (br t, J = 6.3 Hz, 1H), 7.65-7.53 (m, 2H), 3.10 (d, J = 6.2 |
| | | Hz, 2H), 2.06-1.95 (m, 6H), 1.62-1.50 (m, 6H); 510.1 |
| P160 | Preparation P21²⁹ | 2.76 minutes¹⁵; 420.4 (chlorine isotope pattern observed); |
| P161 | Preparation P21²⁹ | 2.95 minutes¹⁵; 420.4 (chlorine isotope pattern observed) |
| P162 | C49, P1³⁰ | 10.83 (s, 1H), 8.28 (br t, J = 6.2 Hz, 1H), 7.91 (d, J = 9.7 |
| | | Hz, 1H), 7.65-7.54 (m, 2H), 7.09 (d, J = 9.7 Hz, 1H), |
| | | 3.75 (s, 3H), 3.09 (d, J = 6.2 Hz, 2H), 2.00-1.89 (m, 6H), |
| | | 1.58-1.48 (m, 6H); 472.5 |
| P163 | Preparation P21³¹ | 2.50 minutes¹⁵; 455.4 |
| P164 | Preparation P31 | 2.83 minutes¹⁵; 502.3 |
| P165 | Preparation P31 | 2.78 minutes¹⁵, 484.3 |
| P166 | Preparation P21³²; P12 | 2.66 minutes¹⁵, 498.5 |
| P167 | Preparation P31 | 2.83 minutes¹⁵; 490.4 |
| P168 | Preparation P31 | 2.72 minutes¹⁵; 472.4 |
| P169 | Preparation P31³³; C20 | 2.86 minutes¹⁵; 454.4 |
| P170 | Preparation P21³⁴ | 2.52 minutes¹⁵; 464.4 |
| P171 | Preparation P31⁴ | 2.62 minutes¹⁵; 469.4 |
| P172 | Preparation P31⁴ | 3.03 minutes¹⁵; 487.5 |
| P173 | Preparation P31⁴ | 2.37 minutes¹⁵, 464.5 |
| P174 | Preparation P27; P12 | 2.68 minutes¹⁵; 470.4 |
| P175 | Preparation P21³⁵ | 2.43 minutes¹⁵, 466.5 |
| P176 | Preparation P21³⁶; P12 | 2.19 minutes¹⁵, 496.5 |
| P177 | Preparation P175 | 2.81 minutes¹⁵; 520.5 |
| P178 | Preparation P27; P12 | 2.76 minutes¹⁵; 484.5 |
| P179 | Preparation P27; P12 | 1.90 minutes¹⁵, 484.4 |
| P180 | Preparation P21³⁷; P12 | 1.44 minutes¹⁵; 544.5 (chlorine isotope pattern observed) |
| P181 | Preparation P27; P12 | 2.42 minutes¹⁵; 471.4 |
| P182 | Preparation P27; P12 | 1.88 minutes¹⁵, 470.4 |
| P183 | Preparation P21³⁸ | 2.13 minutes¹⁵; 520.4 |
| P184 | Preparation P162; C48, | 2.50 minutes¹⁵; 490.6 |
| | P2 | |
| P185 | Preparation P30; P7 | 2.70 minutes¹⁵; 510.4 |
| P186 | Preparation P30; P10 | 2.97 minutes¹⁵; 528.3 |
| P187 | Preparation P30; P10 | 2.42 minutes¹⁵; 474.4 |
| P188 | Preparation P30; P10 | 2.78 minutes¹⁵; 510.4 |
| P189 | Preparation P30; P7 | 2.91 minutes¹⁵; 509.4 |
| P190 | Preparation P30; P7 | 2.89 minutes¹⁵; 510.4 |
| P191 | Preparation P30; P7 | 2.91 minutes¹⁵; 510.4 |
| P192 | Preparation P29; P5 | 8.85 (dd, J = 7.0, 1.2 Hz, 1H), 8.48-8.36 (m, 1H), 7.85 |
| | | (br d, J = 8.9 Hz, 1H), 7.63-7.51 (m, 2H), 7.38 (ddd, J = |
| | | 8.9, 6.8, 1 Hz, 1H), 7.33 (br s, 1H), 7.13 (ddd, J = 6.9, 6.9, |
| | | 1.3 Hz, 1H), 3.15 (dd, J = 6, 6 Hz, 2H), 2.86 (tt, J = 12.1, |
| | | 3.6 Hz, 1H), 2.15-2.04 (m, 2H), 1.93-1.81 (m, 2H), |
| | | 1.69-1.43 (m, 3H), 1.21-1.05 (m, 2H); 454.2 |
| P193 | Preparation P20^{20,39} | 10.85 (s, 1H), 9.42 (br s, 1H), 9.39 (br s, 1H), 8.09 (br t, J = |
| | | 6 Hz, 1H), 7.26 (ddd, J = 8, 8, 2.1 Hz, 1H), 6.83 (ddd, J = |
| | | 8, 8, 2 Hz, 1H), 3.09 (d, J = 6.2 Hz, 2H), 2.06-1.95 (m, |
| | | 6H), 1.62-1.51 (m, 6H); 510.2 |
| P194 | Preparation P21⁴⁰ | 11.34 (s, 1H), 9.42 (br s, 1H), 9.39 (br s, 1H), 8.20 (br t, J = |
| | | 6 Hz, 1H), 7.28 (ddd, J = 11.1, 6.2, 2.3 Hz, 1H), 3.09 (d, |
| | | J = 6.3 Hz, 2H), 2.06-1.96 (m, 6H), 1.62-1.51 (m, 6H); 528.2 |
| P195 | Preparation P31⁴¹ | 9.41 (br s, 1H), 9.39 (br s, 1H), 8.03-7.94 (m, 1H), 7.40 |
| | | (dd, J = 11.3, 6.9 Hz, 1H), 6.79 (dd, J = 11.6, 7.1 Hz, 1H), |
| | | 3.09 (d, J = 6.2 Hz, 2H), 2.05-1.95 (m, 6H), 1.61-1.50 |
| | | (m, 6H); 510.2 |
| P196 | Preparation P20 | 8.51 (AB quartet, J_{AB} = 8.8 Hz, Δv_{AB} = 27.2 Hz, 2H), 8.13- |
| | | 8.03 (m, 1H), 7.26 (ddd, J = 8, 8, 2.2 Hz, 1H), 6.86- |
| | | 6.78 (m, 1H), 3.09 (d, J = 6.3 Hz, 2H), 2.08-1.96 (m, |
| | | 6H), 1.63-1.52 (m, 6H); 510.2 |
| P197 | Preparation P20⁴²; P1 | 10.84 (s, 1H), 8.64 (s, 1H), 8.47 (br t, J = 5.8 Hz, 1H), |
| | | 8.21-8.17 (m, 1H), 7.80 (d, J = 9.0 Hz, 1H), 7.64-7.54 |
| | | (m, 2H), 7.43 (dd, J = 9.1, 1.8 Hz, 1H), 4.62-4.50 (m, |
| | | 1H), 3.18 (dd, J = 6, 6 Hz, 2H), 2.21-2.11 (m, 2H), 1.99- |
| | | 1.83 (m, 4H), 1.75-1.61 (m, 1H), 1.31-1.14 (m, 2H); 454.1 |
| P198 | Preparation P19⁴² | 11.38 (br s, 1H), 8.65 (s, 1H), 8.37-8.31 (m, 1H), 8.19 |
| | | (br s, 1H), 7.80 (d, J = 9.0 Hz, 1H), 7.43 (dd, J = 9.1, 1.8 |
| | | Hz, 1H), 7.30 (ddd, J = 11.1, 6.3, 2.3 Hz, 1H), 4.62-4.49 |
| | | (m, 1H), 3.18 (dd, J = 6, 6 Hz, 2H), 2.22-2.12 (m, 2H), |
| | | 2.00-1.85 (m, 4H), 1.75-1.60 (m, 1H), 1.31-1.15 (m, |
| | | 2H); 472.2 |
| P199 | Preparation P19⁴² | 10.90 (s, 1H), 8.64 (s, 1H), 8.21-8.17 (m, 1H), 8.17- |
| | | 8.11 (m, 1H), 7.80 (d, J = 9.0 Hz, 1H), 7.47-7.39 (m, |
| | | 2H), 6.80 (dd, J = 11.6, 7.1 Hz, 1H), 4.61-4.49 (m, 1H), |
| | | 3.17 (dd, J = 6, 6 Hz, 2H), 2.22-2.11 (m, 2H), 1.99- |
| | | 1.84 (m, 4H), 1.75-1.60 (m, 1H), 1.31-1.14 (m, 2H); 454.1 |
| P200 | Preparation P31⁴³ | 11.35 (br s, 1H), 8.51 (AB quartet, J_{AB} = 8.9 Hz, Δv_{AB} = |
| | | 26.9 Hz, 2H), 8.20 (br t, J = 6 Hz, 1H), 7.28 (ddd, J = |
| | | 11.0, 6.2, 2.3 Hz, 1H), 3.09 (d, J = 6.2 Hz, 2H), 2.08- |
| | | 1.96 (m, 6H), 1.63-1.51 (m, 6H); 528.2 |
| P201 | Preparation P19 | 8.51 (AB quartet, J_{AB} = 8.9 Hz, Δv_{AB} = 27.2 Hz, 2H), 8.03- |
| | | 7.92 (m, 1H), 7.40 (dd, J = 11.3, 6.9 Hz, 1H), 6.79 (dd, J = |
| | | 11.6, 7.0 Hz, 1H), 3.09 (d, J = 6.3 Hz, 2H), 2.07-1.95 |
| | | (m, 6H), 1.61-1.51 (m, 6H); 510.1 |
| P202 | Preparation P19⁴⁴ | 8.12-8.04 (m, 1H), 7.71-7.69 (m, 1H), 7.67 (d, half of |
| | | AB quartet, J = 8.1 Hz, 1H), 7.53 (dd, component of ABX |
| | | system, J = 8.1, 1.9 Hz, 1H), 7.40 (dd, J = 11.4, 6.9 Hz, |
| | | 1H), 6.77 (dd, J = 11.7, 7.1 Hz, 1H), 4.44 (s, 2H), 3.98 (tt, |
| | | J = 12.1, 3.9 Hz, 1H), 3.12 (dd, J = 6, 6 Hz, 2H), 1.90- |
| | | 1.71 (m, 4H), 1.63-1.47 (m, 3H), 1.19-1.04 (m, 2H); |
| | | 435.1 (chlorine isotope pattern observed) |
| P203 | Preparation P20⁴⁵ | 9.04-9.01 (m, 1H), 8.56 (s, 1H), 8.47-8.40 (m, 1H), |
| | | 7.79 (d, J = 1.2 Hz, 1H), 7.62-7.51 (m, 2H), 4.68-4.56 |
| | | (m, 1H), 3.17 (dd, J = 6, 6 Hz, 2H), 2.22-2.12 (m, 2H), |
| | | 1.99-1.84 (m, 4H), 1.74-1.60 (m, 1H), 1.30-1.14 (m, |
| | | 2H); 421.1 (chlorine isotope pattern observed) |
| P204 | Preparation P24; P15 | 10.86 (br s, 1H), 9.35 (d, J = 1.3 Hz, 1H), 8.84 (s, 1H), |
| | | 8.83-8.79 (m, 2H), 8.58 (br s, 1H), 8.49 (br t, J = 5.8 Hz, |
| | | 1H), 8.45 (br d, J = 6 Hz, 2H), 7.65-7.54 (m, 2H), 4.70- |
| | | 4.56 (m, 1H), 3.19 (dd, J = 6, 6 Hz, 2H), 2.26-2.15 (m, |
| | | 2H), 2.02-1.87 (m, 4H), 1.77-1.62 (m, 1H), 1.32-1.17 |
| | | (m, 2H); 464.1 |
| P205 | Preparation P24; P15 | 10.85 (s, 1H), 9.34 (br s, 1H), 9.27 (d, J = 1.3 Hz, 1H), |
| | | 8.76 (br s, 1H), 8.59 (br s, 1H), 8.53-8.45 (m, 2H), 8.24- |
| | | 8.21 (m, 1H), 7.64-7.55 (m, 2H), 7.50 (dd, J = 8.2, 4.6 |
| | | Hz, 1H), 4.64-4.52 (m, 1H), 3.19 (dd, J = 6, 6 Hz, 2H), |
| | | 2.25-2.13 (m, 2H), 2.01-1.85 (m, 4H), 1.76-1.61 (m, |
| | | 1H), 1.31-1.16 (m, 2H); 464.1 |
| P206 | Preparation P142⁴⁶; P7 | ¹H NMR (400 MHz, chloroform-d) δ 8.17 (br d, J = 7.7 Hz, |
| | | 1H), 8.11-8.09 (m, 1H), 7.66 (dd, J = 8.2, 7.9 Hz, 1H), |
| | | 7.56 (br dd, component of ABX system, J = 8.2, 2.3 Hz, |
| | | 1H), 7.43-7.33 (m, 2H), 6.00 (br t, J = 6 Hz, 1H), 5.78 (br |
| | | s, 1H), 3.29 (d, J = 6.4 Hz, 2H), 2.07-1.97 (m, 6H), 1.67- |
| | | 1.56 (m, 6H); 538.1 |
| P207 | Preparation P20²⁰; P1 | 10.86 (br s, 1H), 9.42-9.40 (m, 1H), 9.39-9.37 (m, 1H), |
| | | 8.30 (br t, J = 6.3 Hz, 1H), 7.65-7.55 (m, 2H), 3.10 (d, J = |
| | | 6.2 Hz, 2H), 2.04-1.94 (m, 6H), 1.60-1.50 (m, 6H); 510.2 |
| P208 | P1⁴⁷ | 10.81 (br s, 1H), 8.17 (t, J = 6.3 Hz, 1H), 7.62-7.51 (m, |
| | | 2H), 6.30 (br s, 1H), 2.98 (d, J = 6.2 Hz, 2H), 1.74-1.64 |
| | | (m, 6H), 1.45-1.37 (m, 6H), 1.34 (s, 9H); 411.2 |
| P209 | Preparation P38; P8 | 8.64 (s, 1H), 8.18-8.14 (m, 1H), 8.02 (br s, 1H), 7.80 (d, |
| | | J = 9.1 Hz, 1H), 7.42 (dd, J = 9.1, 1.8 Hz, 1H), 7.22 (ddd, |
| | | J = 11.6, 6.7, 2.2 Hz, 1H), 3.11 (d, J = 6.3 Hz, 2H), 2.23- |
| | | 2.13 (m, 6H), 1.72-1.62 (m, 6H); 498.1 |
| P210 | Preparation P18^{21,48} | 10.83 (br s, 1H), 8.29 (br t, J = 6.3 Hz, 1H), 7.90-7.81 |
| | | (m, 2H), 7.65-7.55 (m, 2H), 7.44 (dd, J = 7.3, 1.5 Hz, |
| | | 1H), 3.09 (d, J = 6.2 Hz, 2H), 2.55 (s, 3H), 2.01-1.91 (m, |
| | | 6H), 1.59-1.49 (m, 6H); 455.2 |
| P211 | Preparation P21⁴⁹ | ¹H NMR (400 MHz, methanol-d₄) δ 9.34 (br s, 1H), 8.64 |
| | | (dd, J = 8.2, 2.1 Hz, 1H), 7.98 (d, J = 8.2 Hz, 1H), 7.98- |
| | | 7.90 (m, 1H), 7.45 (dd, J = 11.3, 6.9 Hz, 1H), 6.75 (dd, J = |
| | | 12.1, 6.9 Hz, 1H), 3.25-3.21 (m, 2H), 2.15-2.05 (m, |
| | | 6H), 1.73-1.62 (m, 6H); 509.3 |
| P212 | Preparation P21⁴⁹ | ¹H NMR (400 MHz, methanol-d₄) δ 9.34 (br s, 1H), 8.65 |
| | | (dd, J = 8.2, 2.0 Hz, 1H), 8.17-8.08 (m, 1H), 7.98 (d, J = |
| | | 8.2 Hz, 1H), 7.25 (ddd, J = 11.0, 6.3, 2.3 Hz, 1H), 3.26- |
| | | 3.21 (m, 2H), 2.16-2.06 (m, 6H), 1.73-1.63 (m, 6H); 527.3 |
| P213 | Preparation P21⁴⁹ | ¹H NMR (400 MHz, methanol-d₄) δ 9.37-9.33 (m, 1H), |
| | | 8.65 (dd, J = 8.2, 2.1 Hz, 1H), 7.99 (d, J = 8.2 Hz, 1H), |
| | | 7.47 (dd, J = 11.3, 6.9 Hz, 1H), 6.74 (dd, J = 12.1, 6.9 Hz, |
| | | 1H), 3.36-3.26 (m, 2H, assumed; completely obscured |
| | | by solvent peak), 3.15-3.04 (m, 1H), 2.33-2.24 (m, |
| | | 2H), 2.06-1.96 (m, 2H), 1.80-1.64 (m, 3H), 1.32-1.18 |
| | | (m, 2H); 483.1 |
| P214 | Preparation P19; C34 | 9.50 (s, 2H), 8.15-8.05 (m, 1H), 7.42 (dd, J = 11.4, 6.9 |
| | | Hz, 1H), 6.79 (dd, J = 11.5, 7.1 Hz, 1H), 3.19-3.07 (m, |
| | | 3H), 2.26-2.15 (m, 2H), 1.93-1.82 (m, 2H), 1.68-1.51 |
| | | (m, 3H), 1.22-1.07 (m, 2H); 484.1 |
| P215 | Preparation P24⁹ | 10.88 (br s, 1H), 9.31-9.26 (m, 2H), 8.64 (br s, 1H), 8.55 |
| | | (br d, J = 4.6 Hz, 1H), 8.48 (br t, J = 5.6 Hz, 1H), 8.44 (br |
| | | d, J = 8.0 Hz, 1H), 8.31 (d, J = 1.5 Hz, 1H), 7.64-7.54 |
| | | (m, 2H), 7.49 (dd, J = 8.0, 4.7 Hz, 1H), 4.68-4.57 (m, |
| | | 1H), 3.19 (dd, J = 6, 6 Hz, 2H), 2.25-2.14 (m, 2H), 2.03- |
| | | 1.87 (m, 4H), 1.77-1.62 (m, 1H), 1.32-1.16 (m, 2H); 464.1 |
| P216 | Preparation P24^{9,2} | 10.85 (s, 1H), 9.21 (br s, 1H), 8.55 (br s, 1H), 8.49 (br t, J = |
| | | 5.8 Hz, 1H), 8.20 (s, 1H), 7.97 (s, 1H), 7.90 (br s, 1H), |
| | | 7.65-7.54 (m, 2H), 4.67-4.53 (m, 1H), 3.88 (s, 3H), |
| | | 3.22-3.15 (m, 2H), 2.23-2.13 (m, 2H), 2.01-1.86 (m, |
| | | 4H), 1.77-1.62 (m, 1H), 1.31-1.15 (m, 2H); 467.1 |
| P217 | Preparation P24^{9,2} | ¹H NMR (400 MHz, DMSO-d₆), characteristic peaks: δ |
| | | 9.27 (br s, 1H), 9.24 (s, 2H), 8.64 (br s, 1H), 8.45-8.37 |
| | | (m, 1H), 8.27 (d, J = 1.5 Hz, 1H), 7.60-7.49 (m, 2H), |
| | | 4.68-4.56 (m, 1H), 3.98 (s, 3H), 2.25-2.13 (m, 2H), |
| | | 2.03-1.87 (m, 4H), 1.77-1.61 (m, 1H); 495.1 |
| P218 | Preparation P141; P1 | 8.37 (d, J = 1.0 Hz, 1H), 8.30-8.23 (m, 1H), 7.65 (ddd, J = |
| | | 8.4, 1, 1 Hz, 1H), 7.63-7.53 (m, 3H), 7.19 (ddd, J = |
| | | 8.7, 6.6, 1.2 Hz, 1H), 6.99 (ddd, J = 8.3, 6.6, 0.9 Hz, 1H), |
| | | 3.12 (d, J = 6.2 Hz, 2H), 2.20-2.10 (m, 6H), 1.70-1.61 |
| | | (m, 6H); 412.2 |
| P219 | Preparation P19⁵⁰; C32, | 10.84 (br s, 1H), 9.48 (s, 2H), 8.34-8.24 (m, 1H), 7.65- |
| | P1 | 7.53 (m, 2H), 3.10 (d, J = 6.2 Hz, 2H), 2.03-1.93 (m, |
| | | 6H), 1.60-1.50 (m, 6H); 510.1 |
| P220 | Preparation P24⁵¹; P1 | 10.85 (s, 1H), 8.35-8.28 (m, 2H), 8.14 (s, 1H), 7.90 (br |
| | | s, 1H), 7.77-7.73 (m, 1H), 7.66-7.56 (m, 3H), 7.24 (dd, |
| | | J = 8.7, 1.5 Hz, 1H), 3.86 (s, 3H), 3.12 (d, J = 6.2 Hz, 2H), |
| | | 2.20-2.10 (m, 6H), 1.71-1.60 (m, 6H); 492.1 |
| P221 | P1⁵² | 8.45 (s, 1H), 8.17-8.08 (m, 1H), 7.84 (br s, 1H), 7.65 (d, |
| | | J = 8.8 Hz, 1H), 7.55-7.42 (m, 2H), 7.10 (dd, J = 8.8, 1.7 |
| | | Hz, 1H), 3.10 (d, J = 6.3 Hz, 2H), 2.18-2.09 (m, 6H), |
| | | 1.69-1.59 (m, 6H); 490.0 (bromine isotope pattern |
| | | observed) |
| P222 | Preparation P220 | 10.86 (br s, 1H), 9.20 (s, 2H), 9.18 (s, 1H), 8.47 (d, J = 1.0 |
| | | Hz, 1H), 8.32 (br t, J = 6.3 Hz, 1H), 8.10-8.07 (m, 1H), |
| | | 7.83 (dd, J = 8.7, 0.9 Hz, 1H), 7.66-7.55 (m, 2H), 7.44 |
| | | (dd, J = 8.7, 1.6 Hz, 1H), 3.13 (d, J = 6.2 Hz, 2H), 2.24- |
| | | 2.13 (m, 6H), 1.73-1.62 (m, 6H); 490.1 |
| P223 | Preparation P23⁵¹ | 10.84 (br s, 1H), 8.91 (d, J = 4.8 Hz, 2H), 8.68-8.64 (m, |
| | | 1H), 8.46 (d, J = 1.0 Hz, 1H), 8.33 (br t, J = 6.3 Hz, 1H), |
| | | 8.08 (dd, J = 8.8, 1.4 Hz, 1H), 7.78 (dd, J = 8.9, 1 Hz, 1H), |
| | | 7.67-7.56 (m, 2H), 7.43 (t, J = 4.8 Hz, 1H), 3.13 (d, J = |
| | | 6.2 Hz, 2H), 2.25-2.14 (m, 6H), 1.73-1.63 (m, 6H); 490.1 |
| P224 | Preparation P23⁵¹ | 9.34 (d, J = 1.6 Hz, 1H), 8.72 (dd, J = 2.6, 1.5 Hz, 1H), |
| | | 8.59 (d, J = 2.5 Hz, 1H), 8.48-8.46 (m, 1H), 8.43-8.41 |
| | | (m, 1H), 8.28 (br t, J = 6.2 Hz, 1H), 7.83-7.80 (m, 2H), |
| | | 7.64-7.53 (m, 2H), 3.13 (d, J = 6.2 Hz, 2H), 2.25-2.13 |
| | | (m, 6H), 1.73-1.62 (m, 6H); 490.1 |
| P225 | Preparation P24; P16 | 11.40 (br s, 1H), 8.32 (d, J = 0.9 Hz, 1H), 8.21-8.13 (m, |
| | | 1H), 8.15 (s, 1H), 7.90 (br s, 1H), 7.75 (br s, 1H), 7.64 |
| | | (dd, J = 8.6, 0.9 Hz, 1H), 7.27 (ddd, J = 11.0, 6.4, 2.2 Hz, |
| | | 1H), 7.24 (dd, J = 8.6, 1.5 Hz, 1H), 3.87 (s, 3H), 3.11 (d, J = |
| | | 6.2 Hz, 2H), 2.22-2.09 (m, 6H), 1.72-1.61 (m, 6H); 510.1 |
| P226 | Preparation P20⁵³ | 11.37 (br s, 1H), 9.19-9.15 (m, 1H), 8.45 (dd, |
| | | component of ABX system, J = 8.4, 2.4 Hz, 1H), 8.34- |
| | | 8.27 (m, 1H), 8.27 (d, half of AB quartet, J = 8.3 Hz, 1H), |
| | | 7.29 (ddd, J = 11.1, 6.3, 2.4 Hz, 1H), 3.19-3.06 (m, 3H), |
| | | 2.25-2.15 (m, 2H), 1.93-1.83 (m, 2H), 1.67-1.52 (m, |
| | | 3H), 1.22-1.09 (m, 2H); 501.1 |

1. Reaction of P3 with 2-nitrobenzaldehyde, followed by ring closure of the resulting imine with triethyl phosphite and deprotection using hydrogen chloride in 1,4-dioxane, afforded Preparation P38.
2. Trifluoroacetic acid was used for the final deprotection, rather than hydrogen chloride.
3. The requisite 1-[(1r,4r)-4-(6-methoxy-2H-indazol-2-yl)cyclohexyl]methanamine, hydrochloride salt, was prepared using the method described for synthesis of C25 in Preparation P15.
4. In this case, the boronate coupling was catalyzed by tetrakis(triphenylphosphine)palladium(0) rather than [1,1′-bis(diphenylphosphino)ferrocene]dichloropalladium(II).
5. The requisite chloro-substituted 1-[(1r,4r)-4-(2H-indazol-2-yl)cyclohexyl]methanamine, hydrochloride salt, was prepared using the method described for synthesis of C25 in Preparation P15.
6. The requisite 1-{(1r,4r)-4-[6-(pyrimidin-2-yl)-2H-indazol-2-yl]cyclohexyl}methanamine, hydrochloride salt, was prepared using the method described for synthesis of P13 in Preparation P13.
7. The requisite 1-{(1r,4r)-4-[6-(pyrazin-2-yl)-2H-indazol-2-yl]cyclohexyl}methanamine, hydrochloride salt, was prepared using the method described for synthesis of P13 in Preparation P13.
8. Synthesis of tert-butyl {[(1r,4r)-4-(6-bromoimidazo[1,2-a]pyridin-2-yl)cyclohexyl]methyl}carbamate was carried out from C19, using the method described for synthesis of P11 in Preparation P11. This material was converted to the requisite 1-{(1r,4r)-4-[6-(1-methyl-1H-pyrazol-4-yl)imidazo[1,2-a]pyridin-2-yl]cyclohexyl}methanamine, hydrochloride salt, by reaction with 1-methyl-4-(4,4,5,5-tetramethyl-1,3,2-dioxaborolan-2-yl)-1H-pyrazole in the presence of [1,1′-bis(diphenylphosphino)ferrocene]dichloropalladium(II) and potassium carbonate, followed by deprotection with hydrogen chloride.
9. The requisite N-{[(1r,4r)-4-(5-chloro-2H-pyrazolo[3,4-c]pyridin-2-yl)cyclohexyl]methyl}-3,5-difluoro-4-[(4-methoxyphenyl)methoxy]benzamide was synthesized according to the method described for preparation of P15 in Preparation P15.
10. Cyclopropylmethanol was deprotected with sodium hydride in tetrahydrofuran at 0° C., whereupon N-{[(1r,4r)-4-(5-chloro-2H-pyrazolo[3,4-c]pyridin-2-yl)cyclohexyl]methyl}-3,5-difluoro-4-[(4-methoxyphenyl)methoxy]benzamide (see footnote 9) was added, and the reaction mixture was heated at 80° C. for 16 hours. Subsequent deprotection using trifluoroacetic acid provided Preparation 32.
11. Conditions for analytical HPLC. Column: Waters XBridge C18, 2.1×50 mm, 5 μm; Mobile phase A: water containing 0.0375% trifluoroacetic acid; Mobile phase B: acetonitrile containing 0.01875% trifluoroacetic acid; Gradient: 10% B for 0.50 minutes; 10% to 100% B over 3.5 minutes; Flow rate: 0.8 mL/minute.
12. A mixture of N-{[(1r,4r)-4-(5-chloro-2H-pyrazolo[3,4-c]pyridin-2-yl)cyclohexyl]methyl}-3,5-difluoro-4-[(4-methoxyphenyl)methoxy]benzamide (see footnote 9), [6-(trifluoromethyl)pyridin-2-yl]methanol, tris(dibenzylideneacetone)dipalladium(0), 5-(di-tert-butylphosphanyl)-1′,3′,5′-triphenyl-1′H-1,4′-bipyrazole (BippyPhos), and sodium hydroxide was heated in a 4:1 mixture of 2-methylbutan-2-ol and dichloromethane at 105° C. for 16 hours. Subsequent deprotection using trifluoroacetic acid provided Preparation 33.
13. Conditions for analytical HPLC. Column: Waters XBridge C18, 2.1×50 mm, 5 μm; Mobile phase A: water containing 0.0375% trifluoroacetic acid; Mobile phase B: acetonitrile containing 0.01875% trifluoroacetic acid; Gradient: 1% to 5% B over 0.6 minutes; 5% to 100% B over 3.4 minutes; Flow rate: 0.8 mL/minute.
14. Intermediate P14 was reacted with the appropriate aromatic bromide in the presence of [1,1′-bis(diphenylphosphino)ferrocene]dichloropalladium(II) and tripotassium phosphate, followed by deprotection with hydrogen chloride.
15. Column: Waters Atlantis dC18, 4.6×50 mm, 5 μm; Mobile phase A: water containing 0.05% trifluoroacetic acid (v/v); Mobile phase B: acetonitrile containing 0.05% trifluoroacetic acid (v/v); Gradient: 5.0% to 95% B, linear over 4.0 minutes, then 95% B for 1.0 minute; Flow rate: 2 mL/minute.
16. In this case, [1,1′-bis(diphenylphosphino)ferrocene]dichloropalladium(II) was used in place of chloro[(di(1-adamantyl)-N-butylphosphine)-2-(2-aminobiphenyl)]palladium(II) (cataCXium® A Pd G2).
17. The coupling was carried out using the conditions described in Step 1 of Preparation P26; deprotection of the product was then effected using trifluoroacetic acid.
18. In this case, the chloro reactant was used, rather than the bromide.
19. In this case, 5-bromo-1H-pyrrolo[2,3-b]pyridine was reacted with p-toluenesulfonyl chloride in the presence of N,N-diisopropylethylamine, and the resultant 5-bromo-1-(4-methylbenzene-1-sulfonyl)-1H-pyrrolo[2,3-b]pyridine was used in the coupling reaction.
20. In this case, the acyl intermediate was cyclized by treatment with sodium acetate, rather than tetrabutylammonium fluoride.
21. The requisite 4-({3,5-difluoro-4-[(4-methoxyphenyl)methoxy]benzamido}methyl)bicyclo[2.2.2]octane-1-carboxylic acid was synthesized using the method described for preparation of P9 in Preparation P9, but employing P1 as starting material.
22. The final deprotection was carried out using methanesulfonic acid in 1,1,1,3,3,3-hexafluoropropan-2-ol, rather than hydrogen chloride.
23. Conversion of P1 to the requisite N-[(4-aminobicyclo[2.2.2]octan-1-yl)methyl]-3,5-difluoro-4-[(4-methoxyphenyl)methoxy]benzamide was carried out using the method described in Preparation P3. This intermediate was condensed with 2-nitro-5-(trifluoromethyl)benzaldehyde, followed by ring closure of the resulting imine with triethyl phosphite and deprotection using hydrogen chloride, to afford Preparation P141.
24. Reaction of P7 with 4-[(fluorosulfonyl)oxy]benzoic acid (A. Baranczak et al., J. Am. Chem. Soc. 2015, 137, 7404-7414), 1-[3-(dimethylamino)propyl]-3-ethylcarbodiimide hydrochloride, 1H-benzotriazol-1-ol, and N,N-diisopropylethylamine afforded the acyl intermediate; this was subjected to tetrabutylammonium fluoride, providing 3,5-difluoro-N-({4-[5-(4-hydroxyphenyl)-1,2,4-oxadiazol-3-yl]bicyclo[2.2.2]octan-1-yl}methyl)-4-[(4-methoxyphenyl)methoxy]benzamide. Reaction with (4-acetamidophenyl)imidodisulfuryl difluoride (AISF) and cesium carbonate, followed by deprotection via hydrogen chloride treatment, afforded Preparation P142.
25. p-Toluenesulfonic acid was used for the final deprotection, rather than hydrogen chloride.
26. In this case, the acyl intermediate was cyclized by heating in 1-methylpyrrolidin-2-one, rather than treatment with sodium acetate.
27. Methyl 4-(aminomethyl)bicyclo[2.2.2]octane-1-carboxylate was protected by reaction with benzyl chloroformate and triethylamine, whereupon the ester was cleaved using sodium hydroxide. The resulting 4-({[(benzyloxy)carbonyl]amino}methyl)bicyclo[2.2.2]octane-1-carboxylic acid was converted to benzyl {[4-(1,3-benzoxazol-2-yl)bicyclo[2.2.2]octan-1-yl]methyl}carbamate using the method described for synthesis of C26 from P4 in Preparation P17; subsequent hydrogenation over palladium on carbon afforded the requisite 1-[4-(1,3-benzoxazol-2-yl)bicyclo[2.2.2]octan-1-yl]methanamine.
28. The requisite 1-(4-{3-[6-(trifluoromethyl)pyridazin-3-yl]-1,2,4-oxadiazol-5-yl}bicyclo[2.2.2]octan-1-yl)methanamine was prepared using the method described for synthesis of C32 in Preparation P19.
29. The requisite chloro-substituted 1-[(1r,4r)-4-(2H-indazol-2-yl)cyclohexyl]methanamine, hydrochloride salt, was prepared using the method employed for synthesis of C25 in Preparation P15.
30. Reaction of C49 with P1, mediated by O-(7-azabenzotriazol-1-yl)-N,N,N,N′-tetramethyluronium hexafluorophosphate and N,N-diisopropylethylamine, provided 3,5-difluoro-4-[(4-methoxyphenyl)methoxy]-N-({4-[3-(6-methoxypyridazin-3-yl)-1,2,4-oxadiazol-5-yl]bicyclo[2.2.2]octan-1-yl}methyl)benzamide. This material was demethylated using trimethylsilyl chloride and potassium iodide to afford 3,5-difluoro-4-[(4-methoxyphenyl)methoxy]-N-({4-[3-(6-oxo-1,6-dihydropyridazin-3-yl)-1,2,4-oxadiazol-5-yl]bicyclo[2.2.2]octan-1-yl}methyl)benzamide, which was N-methylated using methyl 4-nitrobenzene-1-sulfonate and cesium carbonate; deprotection with trifluoroacetic acid provided Preparation P162.
31. tert-Butyl {[(1r,4r)-4-aminocyclohexyl]methyl}carbamate was converted to the requisite 1-{(1r,4r)-4-[5-(trifluoromethyl)-2H-pyrazolo[3,4-c]pyridin-2-yl]cyclohexyl}methanamine, hydrochloride salt using the procedure described in Preparation P22 for the synthesis of P22 from P3.
32. 1-{(1r,4r)-4-[6-(1-Ethyl-1H-pyrazol-4-yl)-2H-indazol-2-yl]cyclohexyl}methanamine, hydrochloride salt, was prepared from P12 using the method described for synthesis of P13 in Preparation P13.
33. 1-{(1r,4r)-4-[6-(Difluoromethyl)-2H-indazol-2-yl]cyclohexyl}methanamine, trifluoroacetate salt, was prepared from C20 by reaction with 2-(difluoromethanesulfonyl)pyridine and zinc in the presence of nickel(II) chloride ethylene glycol dimethyl ether complex, 4-methylpyridine-2,6-dicarboximidamide (see J. M. E. Hughes and P. S. Fier, Org. Lett. 2019, 21, 5650-5654), and tetraethylammonium iodide; subsequent deprotection was carried out with trifluoroacetic acid.
34. tert-Butyl ({(1r,4r)-4-[5-(4,4,5,5-tetramethyl-1,3,2-dioxaborolan-2-yl)-2H-indazol-2-yl]cyclohexyl}methyl)carbamate was prepared in the same manner as P12 in Preparation P12. It was subsequently converted to the requisite 1-{(1r,4r)-4-[5-(pyrimidin-2-yl)-2H-indazol-2-yl]cyclohexyl}methanamine, hydrochloride salt, using the method described in Preparation P13.
35. tert-Butyl ({(1r,4r)-4-[5-(4,4,5,5-tetramethyl-1,3,2-dioxaborolan-2-yl)-2H-indazol-2-yl]cyclohexyl}methyl)carbamate (see footnote 34) was converted to the requisite 1-{(1r,4r)-4-[5-(1-methyl-1H-pyrazol-4-yl)-2H-indazol-2-yl]cyclohexyl}methanamine, hydrochloride salt, via the method described for synthesis of C41 from P12 in Preparation P26, followed by deprotection with hydrogen chloride.
36. Conversion of P12 to the requisite 2-(4-{2-[(1r,4r)-4-(aminomethyl)cyclohexyl]-2H-indazol-6-yl}-1H-pyrazol-1-yl)ethan-1-ol was carried out using the method described for synthesis of C41 from P12 in Preparation P26, followed by deprotection with hydrogen chloride.
37. Reaction of P12 with 4-bromo-1-(oxetan-3-yl)-1H-pyrazole was carried out using the method described for synthesis of C41 from P12 in Preparation P26. Subsequent deprotection with hydrogen chloride also cleaved the oxetane ring, providing 2-(4-{2-[(1r,4r)-4-(aminomethyl)cyclohexyl]-2H-indazol-6-yl}-1H-pyrazol-1-yl)-3-chloropropan-1-ol.
38. Synthesis of tert-butyl {[(1r,4r)-4-(6-bromoimidazo[1,2-a]pyridin-2-yl)cyclohexyl]methyl}carbamate was carried out from C19, using the method described for synthesis of P11 in Preparation P11. Coupling of this material with 1-(difluoromethyl)-4-(4,4,5,5-tetramethyl-1,3,2-dioxaborolan-2-yl)-1H-pyrazole, in the presence of [1,1′-bis(di-tert-butylphosphino)ferrocene]dichloropalladium(II) and potassium carbonate, followed by deprotection with hydrogen chloride, afforded the requisite 1-[(1r,4r)-4-{6-[1-(difluoromethyl)-1H-pyrazol-4-yl]imidazo[1,2-a]pyridin-2-yl}cyclohexyl]methanamine, hydrochloride salt.
39. In this case, 2,3-difluoro-4-hydroxybenzoic acid was employed for the final amide formation, rather than P1.
40. The requisite 1-(4-{3-[5-(trifluoromethyl)pyrazin-2-yl]-1,2,4-oxadiazol-5-yl}bicyclo[2.2.2]octan-1-yl)methanamine, hydrochloride salt was prepared in the following manner. Reaction of 4-{[(tert-butoxycarbonyl)amino]methyl}bicyclo[2.2.2]octane-1-carboxylic acid and N′-hydroxy-5-(trifluoromethyl)pyrazine-2-carboximidamide was carried out using 1-[3-(dimethylamino)propyl]-3-ethylcarbodiimide hydrochloride and 1-methyl-1H-imidazole; the resulting acyl intermediate was cyclized by heating at 120° C. in 1-methylpyrrolidin-2-one, then deprotected with hydrogen chloride.
41. In this case, hydrogen chloride was used for the intermediate deprotection, rather than trifluoroacetic acid. Additionally, the final coupling was carried out with O-(7-azabenzotriazol-1-yl)-N,N,N,N′-tetramethyluronium hexafluorophosphate and N,N-diisopropylethylamine, rather than the reagents described in Preparation P31.
42. tert-Butyl {[(1r,4r)-4-aminocyclohexyl]methyl}carbamate was condensed with 2-nitro-5-(trifluoromethyl)benzaldehyde, followed by ring closure of the resulting imine with triethyl phosphite and deprotection using hydrogen chloride, to afford the requisite 1-{(1r,4r)-4-[5-(trifluoromethyl)-2H-indazol-2-yl]cyclohexyl}methanamine, hydrochloride salt.
43. In this case, hydrogen chloride was used for the intermediate deprotection, rather than trifluoroacetic acid.
44. Reaction of tert-butyl {[(1r,4r)-4-aminocyclohexyl]methyl}carbamate with methyl 2-(bromomethyl)-4-chlorobenzoate and triethylamine, followed by deprotection with hydrogen chloride, provided the requisite 2-[(1r,4r)-4-(aminomethyl)cyclohexyl]-5-chloro-2,3-dihydro-1H-isoindol-1-one, hydrochloride salt.
45. The requisite 1-[(1r,4r)-4-(5-chloro-2H-pyrazolo[3,4-c]pyridin-2-yl)cyclohexyl]methanamine, hydrochloride salt, was prepared using the method described for synthesis of C25 in Preparation P15.
46. In this case, 3-hydroxybenzoic acid was used in place of 4-[(fluorosulfonyl)oxy]benzoic acid.
47. Treatment of tert-butyl [4-(hydroxymethyl)bicyclo[2.2.2]octan-1-yl]carbamate with methanesulfonyl chloride and triethylamine, followed by displacement of the resulting methanesulfonate group using sodium azide and potassium carbonate, provided tert-butyl [4-(azidomethyl)bicyclo[2.2.2]octan-1-yl]carbamate. This material was hydrogenated over palladium on carbon, and the resulting primary amine was acylated with P1 by reaction with 1-[3-(dimethylamino)propyl]-3-ethylcarbodiimide hydrochloride and 1H-benzotriazol-1-ol. The product was deprotected via hydrogenation over palladium on carbon to provide Preparation P208.
48. In this case, the acyl intermediate was cyclized by treatment with tetrabutylammonium fluoride, rather than sodium acetate.
49. Reaction of 4-{[(tert-butoxycarbonyl)amino]methyl}bicyclo[2.2.2]octane-1-carboxylic acid with N′-hydroxy-6-(trifluoromethyl)pyridine-3-carboximidamide N′-hydroxy-4-(trifluoromethyl)benzene-1-carboximidamide, mediated by 1-[3-(dimethylamino)propyl]-3-ethylcarbodiimide hydrochloride and 1-methyl-1H-imidazole, was followed by heating of the acyl intermediate in 1-methylpyrrolidin-2-one. The resulting cyclized material was deprotected with hydrogen chloride to provide the requisite 1-(4-{3-[6-(trifluoromethyl)pyridin-3-yl]-1,2,4-oxadiazol-5-yl}bicyclo[2.2.2]octan-1-yl)methanamine, hydrochloride salt.
50. In this case, a final deprotection step was carried out using hydrogen chloride.
51. The requisite N-{[4-(6-bromo-2H-indazol-2-yl)bicyclo[2.2.2]octan-1-yl]methyl}-3,5-difluoro-4-[(4-methoxyphenyl)methoxy]benzamide was prepared using the methods described for synthesis of P16 in Preparations P8 and P16.
52. N-{[4-(6-Bromo-2H-indazol-2-yl)bicyclo[2.2.2]octan-1-yl]methyl}-3,5-difluoro-4-[(4-methoxyphenyl)methoxy]benzamide, described in footnote 51, was deprotected using hydrogen chloride to afford Preparation P221.
53. The final coupling was carried out with 2,3,5-trifluoro-4-hydroxybenzoic acid, rather than with P1.

### Preparation P227

### N-{[4-(Bromoacetyl)bicyclo[2.2.2]octan-1-yl]methyl}-2,3,5-trifluoro-4-[(4-methoxyphenyl)methoxy]benzamide (P227)

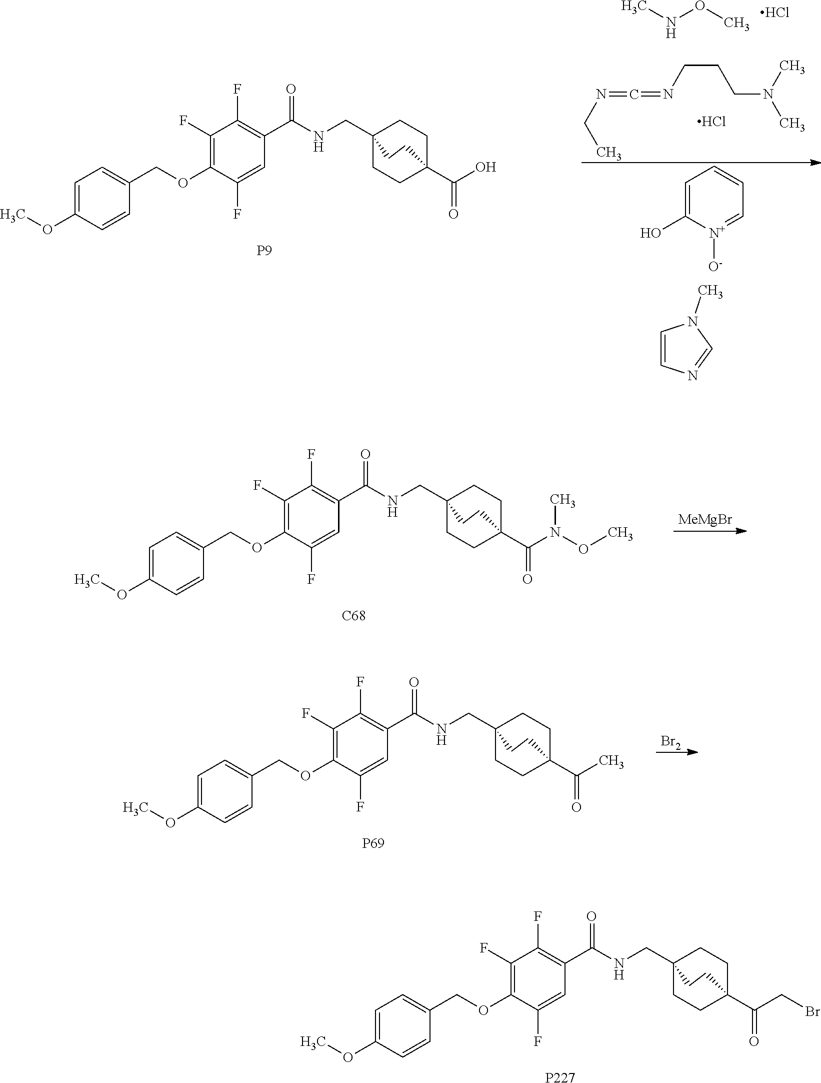

Step 1. Synthesis of N-methoxy-N-methyl-4-({2,3,5-trifluoro-4-[(4-methoxyphenyl)methoxy]benzamido}methyl)bicyclo[2.2.2]octane-1-carboxamide (C68): To a solution of P9 (8.00 g, 16.8 mmol), N-methoxymethanamine hydrochloride (1.96 g, 20.1 mmol), and 2-hydroxypyridine 1-oxide (2.42 g, 21.8 mmol) in N,N-dimethylformamide (100 mL) was added 1-methyl-1H-imidazole (4.13 g, 50.3 mmol). After the mixture had been stirred at 25° C. for 2 minutes, 1-[3-(dimethylamino)propyl]-3-ethylcarbodiimide hydrochloride (3.85 g, 20.1 mmol) was added, and the reaction mixture was stirred at 25° C. for 16 hours. Water (200 mL) was then added, and the resulting mixture was extracted with ethyl acetate (2×200 mL); the combined organic layers were washed with saturated aqueous sodium chloride solution, dried over sodium sulfate, filtered, and concentrated in vacuo. The residue was added to a solution of petroleum ether and ethyl acetate (5:1, 100 mL), whereupon the mixture was stirred for 30 minutes and filtered, affording C68 as a solid. Yield: 5.30 g, 10.2 mmol, 61%. LCMS m/z 521.2 [M+H]⁺. ¹H NMR (400 MHz, DMSO-d₆) δ 8.27 (br t, J=6.2 Hz, 1H), 7.35 (d, J=8.6 Hz, 2H), 7.34-7.28 (m, 1H), 6.94 (d, J=8.7 Hz, 2H), 5.21 (s, 2H), 3.75 (s, 3H), 3.62 (s, 3H), 3.04 (s, 3H), 2.99 (d, J=6.2 Hz, 2H), 1.81-1.71 (m, 6H), 1.44-1.34 (m, 6H).

Step 2. Synthesis of N-[(4-acetylbicyclo[2.2.2]octan-1-yl)methyl]-2,3,5-trifluoro-4-[(4-methoxyphenyl)methoxy]benzamide (C69): A solution of methylmagnesium bromide in tetrahydrofuran (3 M; 10 mL, 30 mmol) was added drop-wise to a −5° C. solution of C68 (5.00 g, 9.60 mmol) in tetrahydrofuran (100 mL), whereupon the reaction mixture was stirred at 25° C. for 4 hours. After the reaction mixture had been treated with aqueous ammonium chloride solution (100 mL), it was extracted with ethyl acetate (3×100 mL), and the combined organic layers were washed with saturated aqueous sodium chloride solution (100 mL) and concentrated in vacuo. Silica gel chromatography (Gradient: 0% to 50% ethyl acetate in petroleum ether) provided C69 as a white solid. Yield: 3.51 g, 7.38 mmol, 77%. LCMS m/z 476.2 [M+H]⁺. ¹H NMR (400 MHz, DMSO-d₆) δ 8.29 (br t, J=6.2 Hz, 1H), 7.35 (d, J=8.7 Hz, 2H), 7.34-7.27 (m, 1H), 6.94 (d, J=8.7 Hz, 2H), 5.21 (s, 2H), 3.75 (s, 3H), 3.01 (d, J=6.2 Hz, 2H), 2.03 (s, 3H), 1.66-1.57 (m, 6H), 1.45-1.35 (m, 6H).

Step 3. Synthesis of N-{[4-(bromoacetyl)bicyclo[2.2.2]octan-1-yl]methyl}-2,3,5-trifluoro-4-[(4-methoxyphenyl)methoxy]benzamide (P227): Bromine (0.148 mL, 459 mg, 2.89 mmol) was added in a portion-wise manner to a 0° C. solution of C69 (1.30 g, 2.73 mmol) and di-tert-butyl dicarbonate (1.19 g, 5.45 mmol) in methanol (15 ml). After the reaction mixture had been stirred at 0° C. for 1 hour, then at 25° C. for 2 hours, N,N-diisopropylethylamine (1.24 g, 9.59 mmol) was added in a portion-wise manner. The reaction mixture was stirred at 25° C. for an additional 20 minutes, whereupon it was concentrated in vacuo; the residue was purified by silica gel chromatography (Gradient: 0% to 30% ethyl acetate in petroleum ether) to provide P227 as a white solid. Yield: 1.34 g, 2.42 mmol, 89%. LCMS m/z 554.2 (bromine isotope pattern observed) [M+H]⁺. ¹H NMR (400 MHz, DMSO-d₆) δ 8.31 (br t, J=6.2 Hz, 1H), 7.35 (d, J=8.6 Hz, 2H), 7.31 (ddd, J=11.0, 6.0, 2.3 Hz, 1H), 6.94 (d, J=8.6 Hz, 2H), 5.21 (s, 2H), 4.57 (s, 2H), 3.75 (s, 3H), 3.00 (d, J=6.3 Hz, 2H), 1.73-1.64 (m, 6H), 1.45-1.35 (m, 6H).

### Preparation P228

### 1-(4-{4-[2-(Methylsulfanyl)pyrimidin-4-yl]-1,3-thiazol-2-yl}bicyclo[2.2.2]octan-1-yl)methanamine (P228)

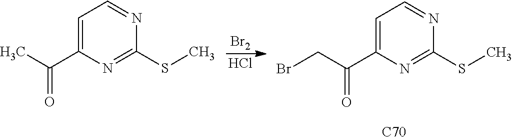

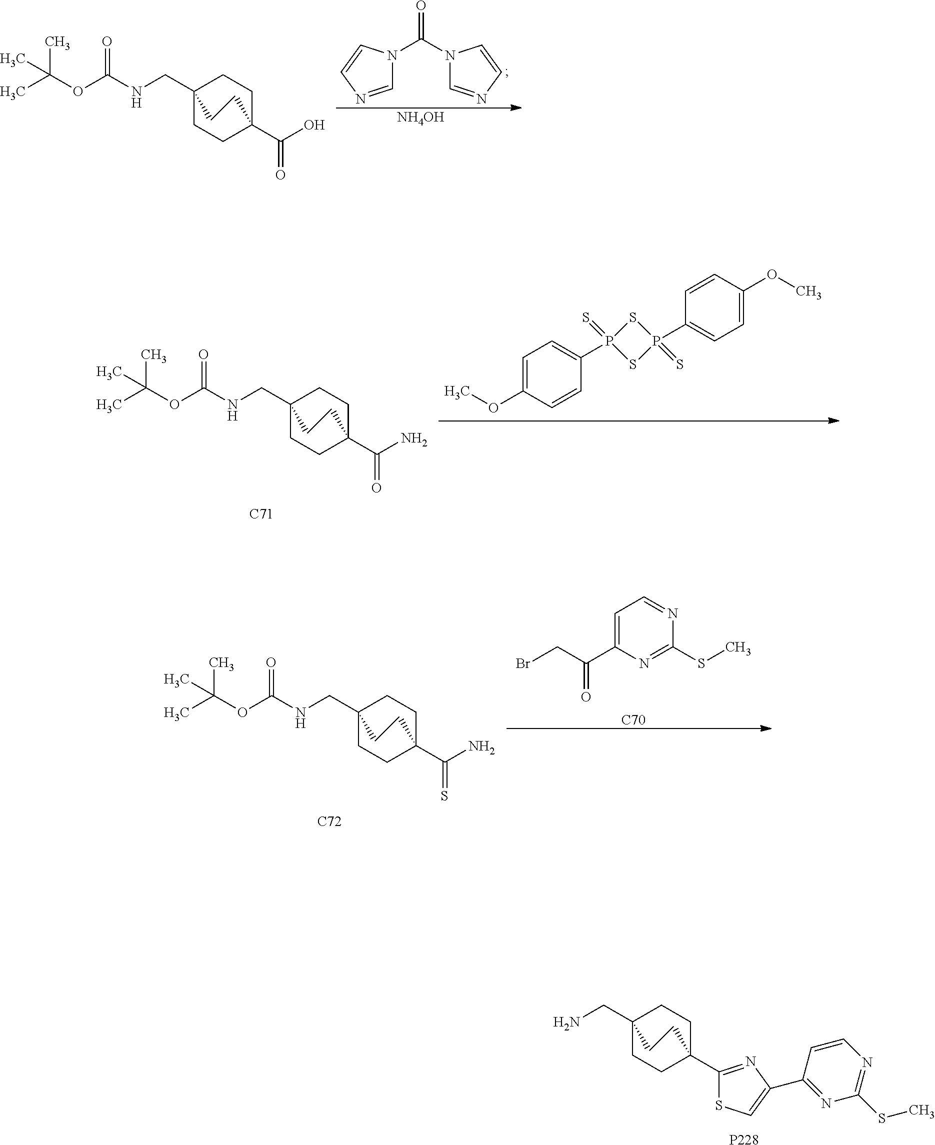

Step 1. Synthesis of 2-bromo-1-[2-(methylsulfanyl)pyrimidin-4-yl]ethan-1-one (C70): To a solution of 1-[2-(methylsulfanyl)pyrimidin-4-yl]ethan-1-one (800 mg, 4.76 mmol) in hydrochloric acid (40%, 28 mL) was added bromine (760 mg, 4.76 mmol), whereupon the reaction mixture was stirred at 25° C. for 8 hours. Solids were collected via filtration; the filter cake was mixed with water (50 mL) and adjusted to pH 7 by addition of saturated aqueous sodium bicarbonate solution. The resulting mixture was extracted with dichloromethane (3×20 mL), and the combined organic layers were dried over sodium sulfate, filtered, and concentrated in vacuo to provide C70 as a yellow solid. Yield: 900 mg, 3.64 mmol, 76%. LCMS m/z 247.0 (bromine isotope pattern observed) [M+H]⁺. ¹H NMR (400 MHz, chloroform-d) δ 8.78 (d, J=4.9 Hz, 1H), 7.57 (d, J=4.9 Hz, 1H), 4.74 (s, 2H), 2.62 (s, 3H).

Step 2. Synthesis of tert-butyl [(4-carbamoylbicyclo[2.2.2]octan-1-yl)methyl]carbamate (C71): 1,1′-Carbonyldiimidazole (2.29 g, 14.1 mmol) was added to a solution of 4-{[(tert-butoxycarbonyl)amino]methyl}bicyclo[2.2.2]octane-1-carboxylic acid (2.00 g, 7.06 mmol) in dichloromethane (30 mL). After the reaction mixture had been stirred at 25° C. for 10 minutes, ammonium hydroxide solution (2 mL) was added, and stirring was continued for 16 hours at 25° C. Aqueous sodium hydroxide solution (2 M; 30 mL) was added; after the resulting mixture had been stirred for 10 minutes, it was extracted with dichloromethane, and the organic layer was washed sequentially with water and saturated aqueous sodium chloride solution, dried over sodium sulfate, filtered, and concentrated in vacuo, affording C71 as a white solid. Yield: 1.75 g, 6.20 mmol, 88%. LCMS m/z 283.3 [M+H]⁺. ¹H NMR (400 MHz, DMSO-d₆) δ 6.87 (br s, 1H), 6.72 (br t, J=6.4 Hz, 1H), 6.67 (br s, 1H), 2.66 (d, J=6.4 Hz, 2H), 1.64-1.53 (m, 6H), 1.37 (s, 9H), 1.32-1.22 (m, 6H).

Step 3. Synthesis of tert-butyl [(4-carbamothioylbicyclo[2.2.2]octan-1-yl)methyl]carbamate (C72): To a mixture of C71 (1.75 g, 6.20 mmol) in toluene (20 mL) was added 2,4-bis(4-methoxyphenyl)-1,3,2,4-dithiadiphosphetane-2,4-dithione (Lawesson's reagent; 3.76 g, 9.30 mmol), whereupon the reaction mixture was stirred for 2 hours at 110° C. It was then extracted with ethyl acetate (2×50 mL), and the combined organic layers were washed with saturated aqueous sodium chloride solution, dried over sodium sulfate, filtered, and concentrated in vacuo. Purification via chromatography on silica gel (Gradient: 0% to 100% ethyl acetate in petroleum ether) provided C72 as a yellow oil. Yield: 420 mg, 1.41 mmol, 23%. LCMS m/z 299.2 [M+H]⁺. ¹H NMR (400 MHz, DMSO-d₆), characteristic peaks: δ 6.73 (br t, J=6.3 Hz, 1H), 2.68 (d, J=6.5 Hz, 2H), 1.77-1.66 (m, 6H), 1.37 (s, 9H), 1.35-1.27 (m, 6H).

Step 4. Synthesis of 1-(4-{4-[2-(methylsulfanyl)pyrimidin-4-yl]-1,3-thiazol-2-yl}bicyclo[2.2.2]octan-1-yl)methanamine (P228): To a solution of C72 (420 mg, 1.41 mmol) in a mixture of 1,4-dioxane (2 mL) and toluene (2 mL) was added C70 (348 mg, 1.41 mmol). The reaction mixture was stirred at 120° C. for 16 hours, whereupon LCMS analysis indicated conversion to P228: LCMS m/z 347.1 [M+H]⁺. After removal of solvents in vacuo, the mixture was extracted with ethyl acetate (3×40 mL), and the combined organic layers were washed with saturated aqueous sodium chloride solution (50 mL) and concentrated in vacuo to provide P228 as a yellow solid. This material was used without further purification. Yield: 230 mg, 0.664 mmol, 47%.

### Preparation P229

### N-{[(1r,4r)-4-(6-Bromo-2H-indazol-2-yl)cyclohexyl]methyl}-2,3,5-trifluoro-4-[(4-methoxyphenyl)methoxy]benzamide (P229)

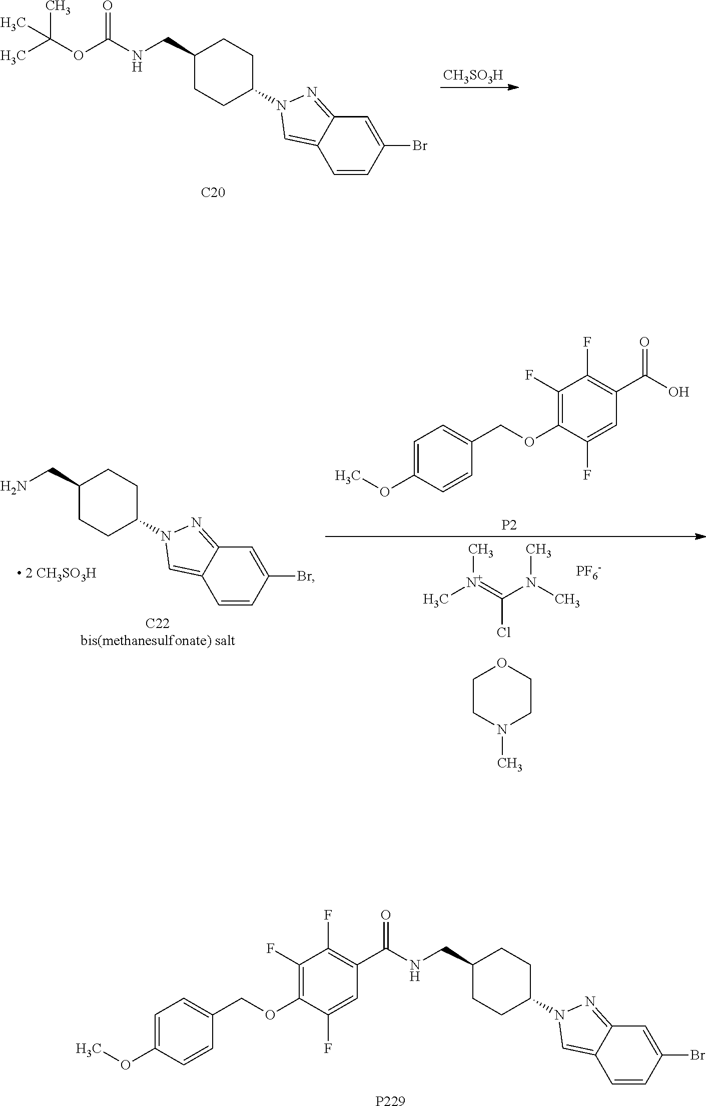

Step 1. Synthesis of 1-[(1r,4r)-4-(6-bromo-2H-indazol-2-yl)cyclohexyl]methanamine, bis(methanesulfonic acid) salt [C22, bis(methanesulfonate) salt]: Methanesulfonic acid (2.75 mL, 42.4 mmol) was added to a solution of C20 (6.18 g, 15.1 mmol) in a mixture of toluene (27 mL) and 1,1,1,3,3,3-hexafluoropropan-2-ol (3 mL), whereupon the reaction mixture was allowed to stir at room temperature for 30 minutes. Removal of solvents in vacuo afforded C22, bis(methanesulfonate) salt as a sticky tan solid (8.93 g). This material was used directly in the following step. LCMS m/z 308.2 (bromine isotope pattern observed) [M+H]⁺.

Step 2. Synthesis of N-{[(1r,4r)-4-(6-bromo-2H-indazol-2-yl)cyclohexyl]methyl}-2,3,5-trifluoro-4-[(4-methoxyphenyl)methoxy]benzamide (P229): 4-Methylmorpholine (9.98 mL, 90.7 mmol) was added to a solution of C22, bis(methanesulfonate) salt (from the previous step; 515.1 mmol) and P2 (4.96 g, 15.9 mmol) in N,N-dimethylformamide (30 mL). Chloro(dimethylamino)-N,N-dimethylmethaniminium hexafluorophosphate (TCFH; 4.25 g, 15.1 mmol) was then added in portions over 5 minutes, and the reaction mixture was allowed to stir at room temperature for 1 hour, whereupon LCMS analysis indicated conversion to P229: LCMS m/z 602.3 (bromine isotope pattern observed) [M+H]⁺. After the reaction mixture had been poured into water, with stirring, the resulting solid was isolated via filtration and washed with water to provide P229 as a light-tan solid. Yield: 8.53 g, 14.2 mmol, 94% over 2 steps. ¹H NMR (400 MHz, chloroform-d) δ 7.85 (br s, 1H), 7.82-7.80 (m, 1H), 7.53 (ddd, J=11.6, 6.8, 2.3 Hz, 1H), 7.45 (br d, J=8.9 Hz, 1H), 7.27 (d, J=8.6 Hz, 2H), 7.08 (dd, J=8.8, 1.6 Hz, 1H), 6.81 (d, J=8.6 Hz, 2H), 6.65-6.52 (m, 1H), 5.18 (s, 2H), 4.32 (tt, J=11.9, 3.8 Hz, 1H), 3.73 (s, 3H), 3.34 (br t, J=6.3 Hz, 2H), 2.33-2.20 (m, 2H), 2.04-1.81 (m, 4H), 1.78-1.64 (m, 1H), 1.30-1.15 (m, 2H).

### Preparation P230

### 2,3,5-Trifluoro-4-[(4-methoxyphenyl)methoxy]-N-({(1r,4r)-4-[6-(4,4,5,5-tetramethyl-1,3,2-dioxaborolan-2-yl)-2H-indazol-2-yl]cyclohexyl}methyl)benzamide (P230)

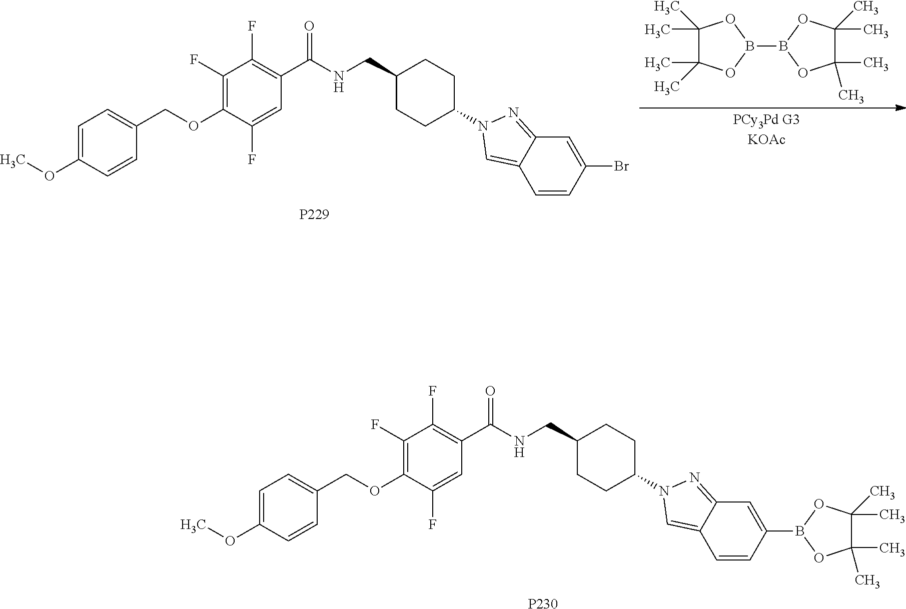

To a flask containing potassium acetate (1.47 g, 15.0 mmol) were added P229 (3.00 g, 4.98 mmol), 4,4,4′,4′,5,5,5′,5′-octamethyl-2,2′-bi-1,3,2-dioxaborolane (1.90 g, 7.48 mmol), [(tricyclohexylphosphine)-2-(2′-aminobiphenyl)]palladium(II) methanesulfonate, dichloromethane adduct (PCy₃Pd G3·dichloromethane; 366 mg, 0.497 mmol), and 1,4-dioxane (36 mL), whereupon the mixture was sparged with nitrogen for approximately 10 minutes. The reaction mixture was heated at 80° C. for 3 hours, cooled to room temperature, and partitioned between ethyl acetate and water. After the aqueous layer had been extracted three times with ethyl acetate, the combined organic layers were washed sequentially with saturated aqueous ammonium chloride solution and saturated aqueous sodium chloride solution, dried over sodium sulfate, filtered, and concentrated in vacuo. Silica gel chromatography (Gradient: 0% to 100% ethyl acetate in hexanes) afforded P230 as a white solid. Yield: 2.77 g, 4.26 mmol, 86%. LCMS m/z 650.5 [M+H]⁺. ¹H NMR (400 MHz, chloroform-d), characteristic peaks: δ 8.27-8.23 (m, 1H), 7.91 (br s, 1H), 7.62 (dd, component of ABX system, J=8.4, 1.1 Hz, 1H), 7.58 (ddd, J=11.7, 6.8, 2.3 Hz, 1H), 7.44 (br d, half of AB quartet, J=8.4 Hz, 1H), 7.34 (d, J=8.6 Hz, 2H), 6.88 (d, J=8.6 Hz, 2H), 6.71-6.60 (m, 1H), 5.24 (s, 2H), 4.44 (tt, J=11.7, 3.8 Hz, 1H), 3.80 (s, 3H), 3.41 (br t, J=6.4 Hz, 2H), 2.39-2.29 (m, 2H), 1.85-1.71 (m, 1H), 1.36 (s, 12H).

### Preparation P231

### 3-[1-(2,6-Dioxopiperidin-3-yl)-3-methyl-2-oxo-2,3-dihydro-1H-benzimidazol-5-yl]propanal (P231)

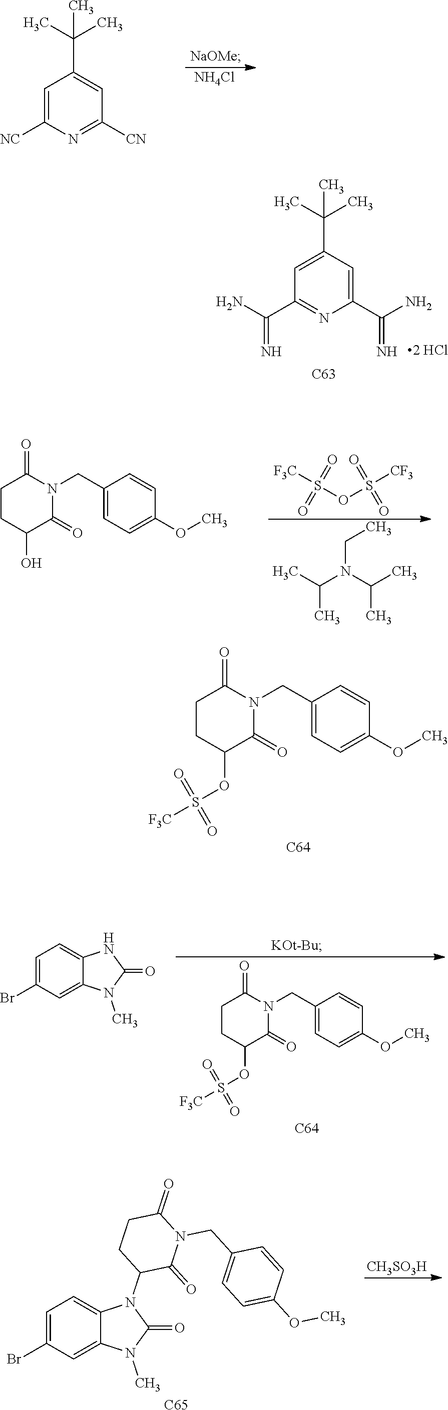

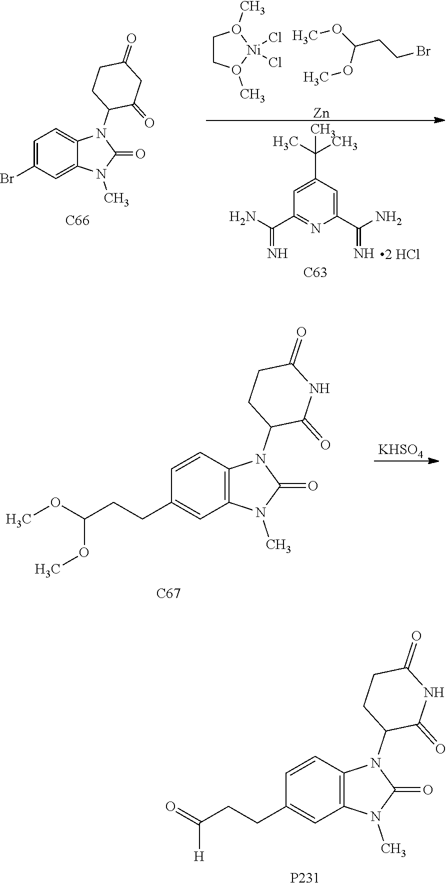

Step 1. Synthesis of 4-tert-butylpyridine-2,6-dicarboximidamide, dihydrochloride salt (C63): To a solution of 4-tert-butylpyridine-2,6-dicarbonitrile (13.0 g, 70.2 mmol) in methanol (150 mL) was added sodium methoxide (0.758 g, 14.0 mmol). The reaction mixture was stirred at 25° C. for 2 hours, whereupon ammonium chloride (11.3 g, 211 mmol) was added and the reaction mixture was stirred at 70° C. for 2 hours. It was then concentrated in vacuo to a volume of approximately 60 mL, and filtered to remove solids. The filtrate was treated with acetonitrile (250 mL) at 20° C., and the resulting mixture was stirred at 20° C. for 25 minutes; collection of the precipitate afforded C63 as a white solid. Yield: 19.0 g, 65.0 mmol, 93%. LCMS m/z 220.2 [M+H]⁺. ¹H NMR (400 MHz, DMSO-d₆) δ 10.16 (br s, 4H), 9.68 (br s, 4H), 8.79 (s, 2H), 1.40 (s, 9H).

Step 2. Synthesis of 1-[(4-methoxyphenyl)methyl]-2,6-dioxopiperidin-3-yl trifluoromethanesulfonate (C64): N,N-Diisopropylethylamine (419 mL, 2.41 mol) was added in one portion to a solution of 3-hydroxy-1-[(4-methoxyphenyl)methyl]piperidine-2,6-dione (300 g, 1.20 mol) in dichloromethane (3.0 L). The resulting mixture was cooled to 0° C. and treated drop-wise with trifluoromethanesulfonic anhydride (298 mL, 1.77 mol), while the reaction mixture was maintained at 0° C. to 5° C. The reaction mixture was then warmed to 20° C. and stirred for 16 hours, whereupon it was diluted with water (2.0 L) at 25° C., and extracted with dichloromethane (3×1.5 L). The combined organic layers were washed with saturated aqueous sodium chloride solution (2 L), dried over sodium sulfate, filtered, and concentrated in vacuo to provide C64 as a yellow oil (458 g). This material was progressed directly to the following step.

Step 3. Synthesis of 3-(5-bromo-3-methyl-2-oxo-2,3-dihydro-1H-benzimidazol-1-yl)-1-[(4-methoxyphenyl)methyl]piperidine-2,6-dione (C65): This reaction was carried out in two parallel batches. Potassium tert-butoxide (68.0 g, 606 mmol) was added portion-wise to a 0° C. to 5° C. solution of 6-bromo-1-methyl-1,3-dihydro-2H-benzimidazol-2-one (125 g, 550 mmol) in tetrahydrofuran (1.2 L). Upon completion of the addition, the reaction mixture was stirred at 0° C. to 5° C. for 1 hour, then treated drop-wise with a solution of C64 (from the previous step; 229 g, 5600 mmol) in tetrahydrofuran (1.8 L) at 0° C. to 5° C. The reaction mixture was warmed to 25° C. and stirred for 20 hours, whereupon it was partitioned between water (3 L) and ethyl acetate (2.5 L). After the aqueous layer had been extracted with ethyl acetate (4×2.5 L), the combined organic layers were washed with saturated aqueous sodium chloride solution (1.5 L). This organic layer was combined with the corresponding one from the second batch, dried over sodium sulfate, filtered, and concentrated in vacuo; silica gel chromatography (Eluent: 3:1 petroleum ether/tetrahydrofuran) afforded C65 as an off-white solid. Combined yield: 205 g, 447 mmol, 41%. ¹H NMR (400 MHz, DMSO-d₆) δ 7.47 (d, J=1.9 Hz, 1H), 7.20 (d, J=8.7 Hz, 2H), 7.20-7.15 (m, 1H), 7.00 (br d, J=8.4 Hz, 1H), 6.85 (d, J=8.7 Hz, 2H), 5.53 (dd, J=13.0, 5.4 Hz, 1H), 4.78 (AB quartet, J_{AB}=14.3 Hz, Δv_{AB}=25.0 Hz, 2H), 3.72 (s, 3H), 3.34 (s, 3H, assumed; partially obscured by water peak), 3.04 (ddd, J=17.3, 13.6, 5.4 Hz, 1H), 2.87-2.65 (m, 2H), 2.12-2.00 (m, 1H).

Step 4. Synthesis of 3-(5-bromo-3-methyl-2-oxo-2,3-dihydro-1H-benzimidazol-1-yl)piperidine-2,6-dione (C66): This reaction was carried out in two parallel batches. Methanesulfonic acid (860 g, 8.95 mol) was added drop-wise to a 20° C. solution of C65 (102.5 g, 223 mmol) in toluene (1 L), whereupon the reaction mixture was heated at 110° C. for 3 hours. It was then poured into water (1.5 L), and the aqueous layer was extracted with ethyl acetate (3×800 mL). After the combined organic layers had been washed with saturated aqueous sodium chloride solution (1.0 L), they were dried over magnesium sulfate and filtered, whereupon the filtrates from the two batches were combined and concentrated in vacuo. Purification using chromatography on silica gel (Gradient: 0% to 100% ethyl acetate in petroleum ether) provided C66 as an off-white solid. Combined yield: 100 g, 296 mmol, 66%. LCMS m/z 338.2 (bromine isotope pattern observed) [M+H]⁺. ¹H NMR (400 MHz, DMSO-d₆) δ 11.12 (br s, 1H), 7.47 (d, J=1.9 Hz, 1H), 7.21 (dd, component of ABX system, J=8.4, 1.9 Hz, 1H), 7.10 (d, half of AB quartet, J=8.4 Hz, 1H), 5.38 (dd, J=12.8, 5.3 Hz, 1H), 3.34 (s, 3H), 2.95-2.82 (m, 1H), 2.76-2.57 (m, 2H), 2.07-1.97 (m, 1H).

Step 5. Synthesis of 3-[5-(3,3-dimethoxypropyl)-3-methyl-2-oxo-2,3-dihydro-1H-benzimidazol-1-yl]piperidine-2,6-dione (C67): A mixture of C66 (20.0 g, 59.1 mmol), nickel(II) chloride, ethylene glycol dimethyl ether complex (1.30 g, 5.91 mmol), C63 (1.77 g, 6.06 mmol), and zinc flakes (7.73 g, 118 mmol) was thoroughly flushed with nitrogen. N,N-Dimethylacetamide (59 mL) was added, followed by 3-bromo-1,1-dimethoxypropane (20.3 mL, 149 mmol), and the reaction mixture was sparged with nitrogen for 5 minutes before being immersed in a 63° C. heating bath. After the reaction mixture had been stirred at 63° C. for 19 hours, it was allowed to cool to room temperature and filtered through a pad of diatomaceous earth. The filter cake was rinsed with ethyl acetate (800 mL), and the combined filtrates were washed with aqueous lithium chloride solution (20%, 300 mL). This resulted in an emulsion; the entire mixture was filtered through diatomaceous earth (see below for further work-up of this filter pad). The ethyl acetate layer of the filtrate was washed with saturated aqueous sodium chloride solution (2×75 mL). The saturated aqueous sodium chloride solution was combined with the earlier aqueous lithium chloride wash, and filtered through diatomaceous earth; this filtrate was extracted with ethyl acetate (2×150 mL). All the organic layers were then combined, dried over magnesium sulfate, filtered through a thin layer of diatomaceous earth, and concentrated under reduced pressure. The resulting solid was suspended in ethyl acetate and slowly concentrated in vacuo, to a volume of approximately 50 mL, whereupon the precipitated solid was collected via filtration and washed with ethyl acetate to provide C67 as a white solid (9.82 g).

The filter cake obtained from filtration of the emulsion above was washed with a mixture of dichloromethane and methanol (9:1, 300 mL). This filtrate was then passed through a pad of silica gel, which was further eluted with a mixture of dichloromethane and methanol (9:1, 300 mL). The combined eluents were concentrated under reduced pressure to provide additional C67 as a white solid (5.12 g). Combined yield: 14.9 g, 41.2 mmol, 70%. LCMS m/z 360.3 [M−H]⁻. ¹H NMR (400 MHz, DMSO-d₆) δ 11.08 (s, 1H), 7.05 (d, J=1.5 Hz, 1H), 7.00 (d, half of AB quartet, J=8.0 Hz, 1H), 6.87 (dd, component of ABX system, J=8.1, 1.5 Hz, 1H), 5.33 (dd, J=12.7, 5.3 Hz, 1H), 4.35 (t, J=5.6 Hz, 1H), 3.32 (s, 3H), 3.25 (s, 6H), 2.90 (ddd, J=16.6, 13.6, 5.5 Hz, 1H), 2.76-2.66 (m, 1H), 2.66-2.56 (m, 3H), 2.04-1.95 (m, 1H), 1.88-1.79 (m, 2H).

Step 6. Synthesis of 3-[1-(2,6-dioxopiperidin-3-yl)-3-methyl-2-oxo-2,3-dihydro-1H-benzimidazol-5-yl]propanal (P231): Aqueous potassium hydrogen sulfate solution (1.0 M; 100 mL, 100 mmol) was added to a vigorously stirring suspension of C67 (7.23 g, 20.0 mmol) in ethyl acetate (120 mL). After the reaction mixture had been stirred at room temperature for 13 hours, solids were collected via filtration. The filter cake was treated with dichloromethane (600 mL); insoluble material was removed via filtration and rinsed with additional dichloromethane (100 mL). The combined dichloromethane filtrates were washed sequentially with saturated aqueous sodium bicarbonate solution (150 mL) and saturated aqueous sodium chloride solution (150 mL), dried over sodium sulfate, and filtered through diatomaceous earth. After this filter cake had been rinsed with dichloromethane (50 mL), the combined filtrates were concentrated in vacuo to afford P231 as a white solid. Yield: 5.23 g, 16.6 mmol, 83%. LCMS m/z 316.3 [M+H]⁺. ¹H NMR (400 MHz, chloroform-d) δ 9.82 (s, 1H), 8.20 (br s, 1H), 6.90 (d, J=8.1 Hz, 1H), 6.87 (s, 1H), 6.72 (d, J=8.0 Hz, 1H), 5.20 (dd, J=13.0, 5.4 Hz, 1H), 3.42 (s, 3H), 3.00 (t, J=7.4 Hz, 2H), 2.97-2.89 (m, 1H), 2.88-2.77 (m, 3H), 2.77-2.64 (m, 1H), 2.28-2.17 (m, 1H).

### Preparation P232

### N-{[4-(7-Bromoimidazo[1,2-a]pyridin-2-yl)bicyclo[2.2.2]octan-1-yl]methyl}-2,3,5-trifluoro-4-[(4-methoxyphenyl)methoxy]benzamide (P232)

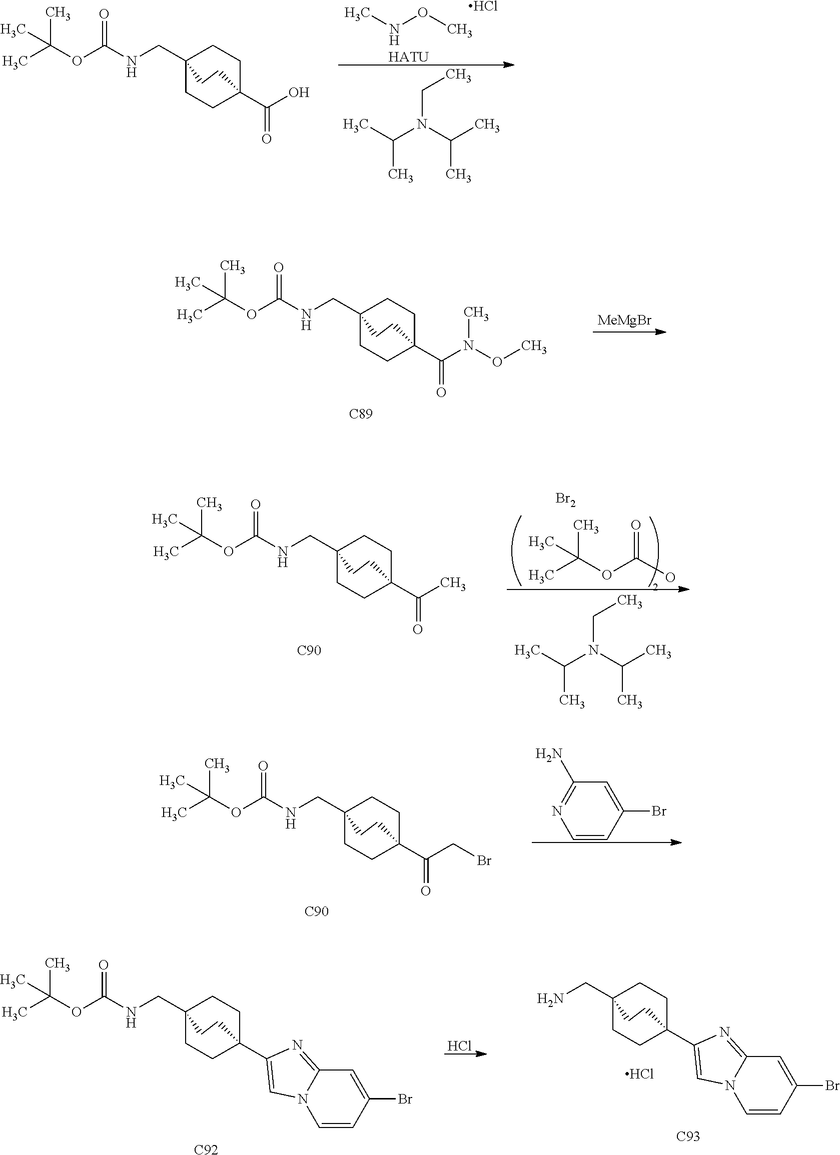

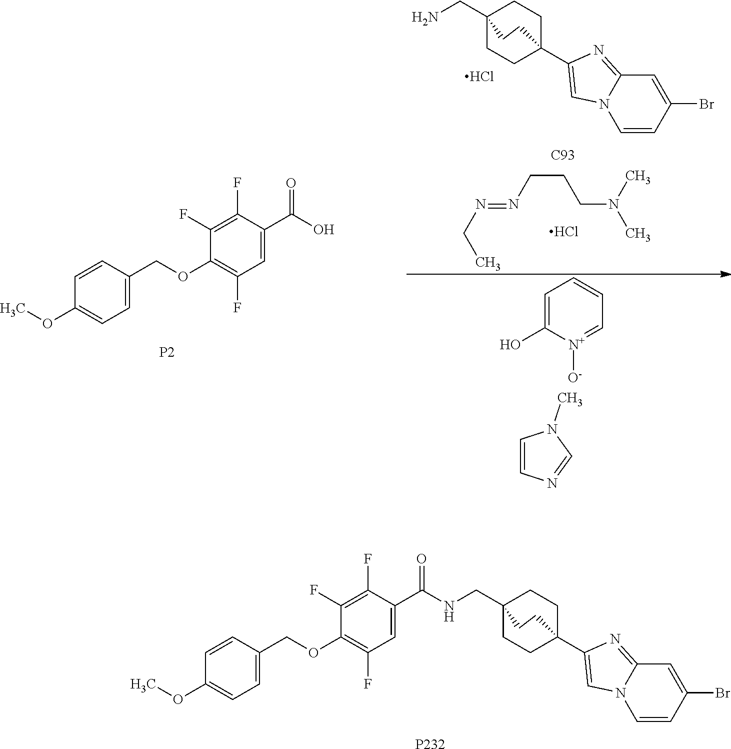

Step 1. Synthesis of tert-butyl ({4-[methoxy(methyl)carbamoyl]bicyclo[2.2.2]octan-1-yl}methyl)carbamate (C89): O-(7-Azabenzotriazol-1-yl)-N,N,N,N′-tetramethyluronium hexafluorophosphate (HATU; 20.1 g, 52.9 mmol) was added to a solution of 4-{[(tert-butoxycarbonyl)amino]methyl}bicyclo[2.2.2]octane-1-carboxylic acid (10.0 g, 35.3 mmol), N-methoxymethanamine hydrochloride (3.79 g, 38.9 mmol), and N,N-diisopropylethylamine (18.4 mL, 106 mmol) in N,N-dimethylformamide (150 mL), and the reaction mixture was stirred at 25° C. for 16 hours. The reaction mixture was then diluted with water (300 mL) and extracted with ethyl acetate (3×100 mL). The combined organic layers were washed with saturated aqueous sodium chloride solution (100 mL), dried over sodium sulfate, filtered, concentrated in vacuo, and purified via chromatography on silica gel (Gradient: 0% to 40% ethyl acetate in petroleum ether) to provide C89 as a yellow gum. Yield: 10.5 g, 32.2 mmol, 91%. LCMS m/z 327.2 [M+H]⁺. ¹H NMR (400 MHz, DMSO-d₆) δ 6.69 (br t, J=6.3 Hz, 1H), 3.62 (s, 3H), 3.03 (s, 3H), 2.67 (d, J=6.3 Hz, 2H), 1.78-1.68 (m, 6H), 1.37 (s, 9H), 1.34-1.25 (m, 6H).

Step 2. Synthesis of tert-butyl [(4-acetylbicyclo[2.2.2]octan-1-yl)methyl]carbamate (C90): This experiment was carried out in two batches. A solution of methylmagnesium bromide in tetrahydrofuran (3 M; 17.2 mL, 51.6 mmol) was added drop-wise to a −5° C. solution of C89 (5.25 g, 16.1 mmol) in tetrahydrofuran (80 mL). The reaction mixture was stirred at 25° C. for 4 hours, treated with aqueous ammonium chloride solution (100 mL), and extracted with ethyl acetate (3×80 mL). The combined organic layers were washed with saturated aqueous sodium chloride solution (100 mL) and concentrated under reduced pressure, whereupon the two batches were combined. Silica gel chromatography (Gradient: 0% to 30% ethyl acetate in petroleum ether) afforded C90 as a colorless solid. Combined yield: 5.90 g, 21.0 mmol, 65%. LCMS m/z 226.2 [(M-2-methylprop-1-ene)+H]⁺. ¹H NMR (400 MHz, DMSO-d₆) δ 6.73 (br t, J=6.3 Hz, 1H), 2.68 (d, J=6.4 Hz, 2H), 2.02 (s, 3H), 1.63-1.54 (m, 6H), 1.37 (s, 9H), 1.34-1.26 (m, 6H).

Step 3. Synthesis of tert-butyl {[4-(bromoacetyl)bicyclo[2.2.2]octan-1-yl]methyl}carbamate (C91): A 0° C. solution of C90 (3.10 g, 11.0 mmol) and di-tert-butyl dicarbonate (4.81 g, 22.0 mmol) in methanol (50 mL) was treated portion-wise with bromine (0.596 mL, 11.6 mmol). The reaction mixture was stirred at 0° C. for 2 hours, followed by 1 hour at 25° C. N,N-Diisopropylethylamine (4.98 g, 38.5 mmol) was then added in a portion-wise manner, and the resulting mixture was stirred at 25° C. for 20 minutes. After removal of volatiles in vacuo, the residue was purified using silica gel chromatography (Gradient: 0% to 30% ethyl acetate in petroleum ether) to provide C91 as a white solid. Yield: 3.50 g, 9.71 mmol, 88%. LCMS m/z 304.1 (bromine isotope pattern observed) [(M-2-methylprop-1-ene)+H]⁺. ¹H NMR (400 MHz, DMSO-d₆) δ 6.74 (brt, J=6.4 Hz, 1H), 4.56 (s, 2H), 2.68 (d, J=6.4 Hz, 2H), 1.71-1.61 (m, 6H), 1.37 (s, 9H), 1.35-1.26 (m, 6H).

Step 4. Synthesis of tert-butyl {[4-(7-bromoimidazo[1,2-a]pyridin-2-yl)bicyclo[2.2.2]octan-1-yl]methyl}carbamate (C92): A solution of C91 (3.50 g, 9.71 mmol) and 4-bromopyridin-2-amine (3.36 g, 19.4 mmol) in ethanol (50 mL) was heated at 70° C. for 16 hours. The reaction mixture was cooled to room temperature, then combined with the product of a similar reaction carried out using C91 (185 mg, 0.513 mmol), poured into water (400 mL) with stirring, and stirred for an additional 20 minutes. The resulting solids were collected via filtration, and washed with water to provide C92 as a light-yellow solid. Combined yield: 4.20 g, 9.67 mmol, 95%. LCMS m/z 434.1 (bromine isotope pattern observed) [M+H]⁺. ¹H NMR (400 MHz, DMSO-d₆) δ 8.40 (dd, J=7.2, 0.8 Hz, 1H), 7.78-7.74 (m, 1H), 7.64-7.62 (br s, 1H), 6.96 (dd, J=7.2, 2.0 Hz, 1H), 6.76 (br t, J=6.3 Hz, 1H), 2.73 (d, J=6.4 Hz, 2H), 1.82-1.73 (m, 6H), 1.45-1.35 (m, 6H), 1.38 (s, 9H).

Step 5. Synthesis of 1-[4-(7-bromoimidazo[1,2-a]pyridin-2-yl)bicyclo[2.2.2]octan-1-yl]methanamine, hydrochloride salt (C93): A solution of hydrogen chloride in 1,4-dioxane (4 M; 50 mL, 200 mmol) was added to a solution of C92 (3.90 g, 8.98 mmol) in methanol (100 mL). The reaction mixture was stirred at 25° C. for 4 hours, then concentrated in vacuo to afford C93 as a white solid. Yield: 3.10 g, 8.36 mmol, 93%. LCMS m/z 334.0 (bromine isotope pattern observed) [M+H]⁺. ¹H NMR (400 MHz, DMSO-d₆) δ 8.78-8.64 (m, 1H), 8.13 (s, 1H), 8.11-7.78 (m, 4H), 7.69-7.54 (m, 1H), 2.69-2.58 (m, 2H), 1.95-1.84 (m, 6H), 1.64-1.53 (m, 6H).

Step 6. Synthesis of N-{[4-(7-bromoimidazo[1,2-a]pyridin-2-yl)bicyclo[2.2.2]octan-1-yl]methyl}-2,3,5-trifluoro-4-[(4-methoxyphenyl)methoxy]benzamide (P232): To a solution of P2 (1.26 g, 4.04 mmol), C93 (1.50 g, 4.05 mmol), and 2-hydroxypyridine 1-oxide (539 mg, 4.85 mmol) in N,N-dimethylformamide (30 mL) was added 1-methyl-1H-imidazole (1.33 g, 16.2 mmol). The reaction mixture was stirred at 25° C. for 5 minutes, and 1-[3-(dimethylamino)propyl]-3-ethylcarbodiimide hydrochloride (1.01 g, 5.27 mmol) was added portion-wise. The mixture was stirred further at 25° C. for 16 hours, whereupon the reaction mixture was poured into water (90 mL). The resulting precipitate was collected via filtration and washed with water (90 mL) to afford P232 as a light-yellow solid. Yield: 2.30 g, 3.66 mmol, 91%. LCMS m/z 628.2 (bromine isotope pattern observed) [M+H]⁺. ¹H NMR (400 MHz, DMSO-d₆) δ 8.40 (br d, J=7.2 Hz, 1H), 8.30 (br t, J=6.2 Hz, 1H), 7.76 (d, J=2.0 Hz, 1H), 7.64 (s, 1H), 7.35 (d, J=8.6 Hz, 2H), 7.35-7.29 (m, 1H), 6.98-6.94 (m, 1H), 6.94 (d, J=8.7 Hz, 2H), 5.21 (s, 2H), 3.75 (s, 3H), 3.05 (d, J=6.2 Hz, 2H), 1.85-1.76 (m, 6H), 1.55-1.46 (m, 6H).

### Preparation P233

### N-{[(1r,4r)-4-Aminocyclohexyl]methyl}-2,3,5-trifluoro-4-[(4-methoxyphenyl)methoxy]benzamide

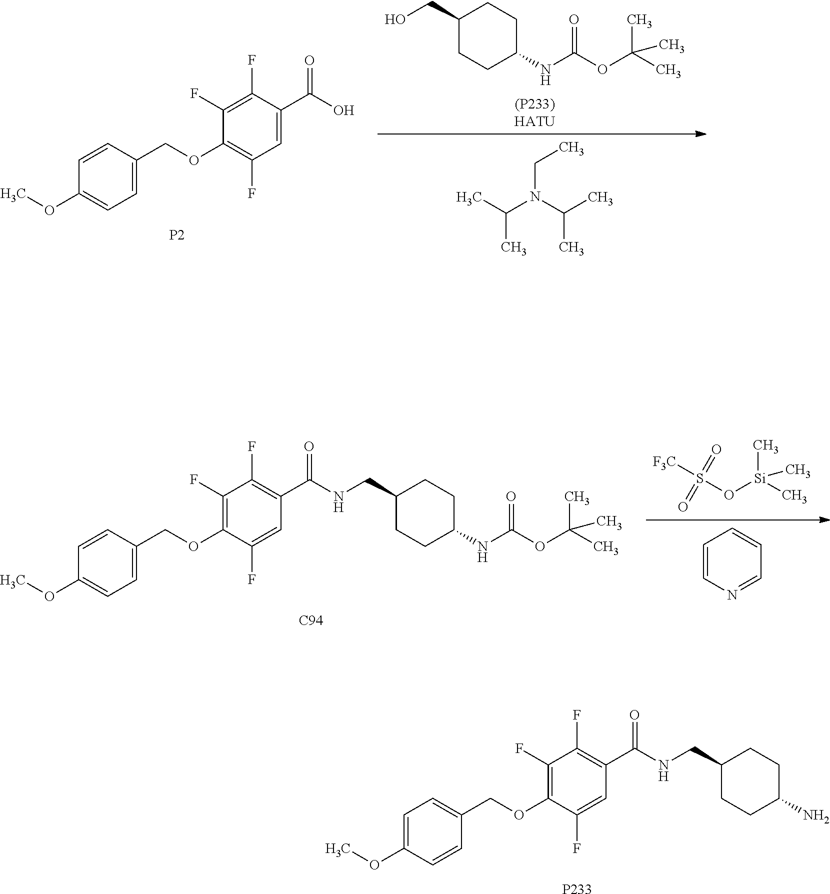

Step 1. Synthesis of tert-butyl [(1r,4r)-4-({2,3,5-trifluoro-4-[(4-methoxyphenyl)methoxy]benzamido}methyl)cyclohexyl]carbamate (C94): To a solution of P2 (5.00 g, 16.0 mmol), N,N-diisopropylethylamine (6.21 g, 48.0 mmol), and O-(7-azabenzotriazol-1-yl)-N,N,N′,N′-tetramethyluronium hexafluorophosphate (HATU; 9.13 g, 24.0 mmol) in N,N-dimethylformamide (75 mL) was added tert-butyl [(1r,4r)-4-(aminomethyl)cyclohexyl]carbamate (3.66 g, 16.0 mmol). After the reaction had been stirred at 25° C. for 2.5 hours, it was combined with a similar reaction carried out using P2 (1.00 g, 3.20 mmol), treated with ice water (200 mL), and stirred for 10 minutes. Filtration of this mixture afforded C94 as a gray solid. Combined yield: 9.00 g, 17.2 mmol, 90%. LCMS m/z 545.2 [M+Na⁺]. ¹H NMR (400 MHz, DMSO-d₆) δ 8.40 (br t, J=6 Hz, 1H), 7.35 (d, J=8.5 Hz, 2H), 7.35-7.29 (m, 1H), 6.94 (d, J=8.6 Hz, 2H), 6.68 (br d, J=8.1 Hz, 1H), 5.21 (s, 2H), 3.75 (s, 3H), 3.21-3.09 (m, 1H), 3.05 (t, J=6.3 Hz, 2H), 1.82-1.66 (m, 4H), 1.47-1.33 (m, 1H), 1.37 (s, 9H), 1.17-1.03 (m, 2H), 1.01-0.87 (m, 2H).

Step 2. Synthesis of N-{[(1r,4r)-4-aminocyclohexyl]methyl}-2,3,5-trifluoro-4-[(4-methoxyphenyl)methoxy]benzamide (P233): Trimethylsilyl trifluoromethanesulfonate (15.3 g, 68.8 mmol) was added drop-wise to a 0° C. mixture of C94 (9.00 g, 17.2 mmol) and pyridine (10.9 g, 140 mmol) in dichloromethane (150 mL). The reaction mixture was stirred at 25° C. for 4 hours, whereupon it was treated with aqueous sodium carbonate solution (100 mL) and aqueous sodium bicarbonate solution (100 mL). After removal of dichloromethane in vacuo, the mixture was filtered and the filter cake was purified using silica gel chromatography (Gradient: 0% to 100% ethyl acetate in petroleum ether, followed by 0% to 20% methanol in dichloromethane), providing P233 as a white solid. Yield: 5.00 g, 11.8 mmol, 69%. LCMS m/z 423.2 [M+H]⁺. ¹H NMR (400 MHz, DMSO-d₆) δ 8.40 (br t, J=6 Hz, 1H), 7.35 (d, J=8.6 Hz, 2H), 7.35-7.29 (m, 1H), 6.94 (d, J=8.6 Hz, 2H), 5.21 (s, 2H), 3.75 (s, 3H), 3.05 (t, J=6.3 Hz, 2H), 2.48-2.39 (m, 1H), 1.80-1.64 (m, 4H), 1.58 (br s, 2H), 1.46-1.34 (m, 1H), 1.02-0.85 (m, 4H).

### Preparation P234

### 2,3,5-Trifluoro-4-[(4-methoxyphenyl)methoxy]-N-({(1r,4r)-4-[5-(piperazin-1-yl)-2H-pyrazolo[4,3-b]pyridin-2-yl]cyclohexyl}methyl)benzamide (P234)

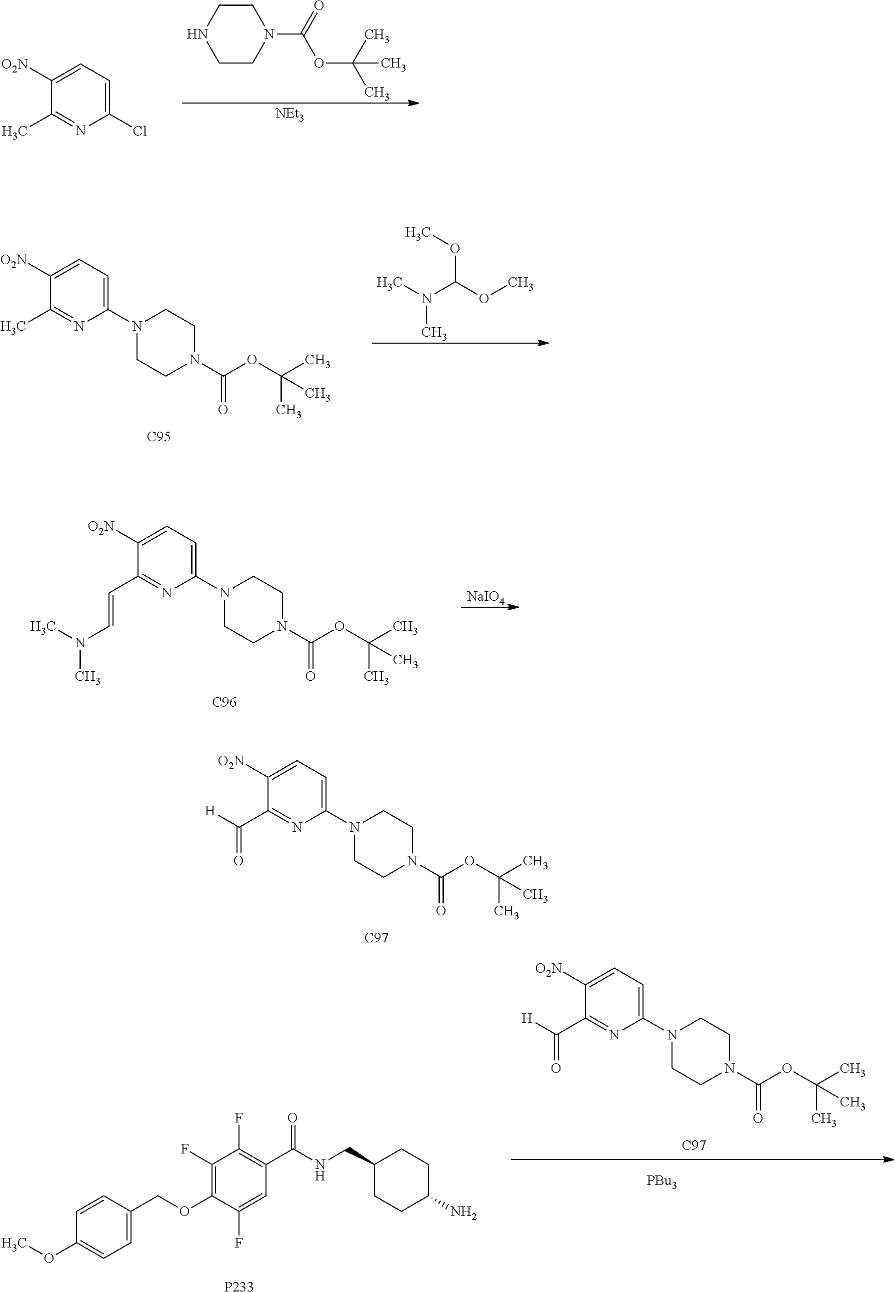

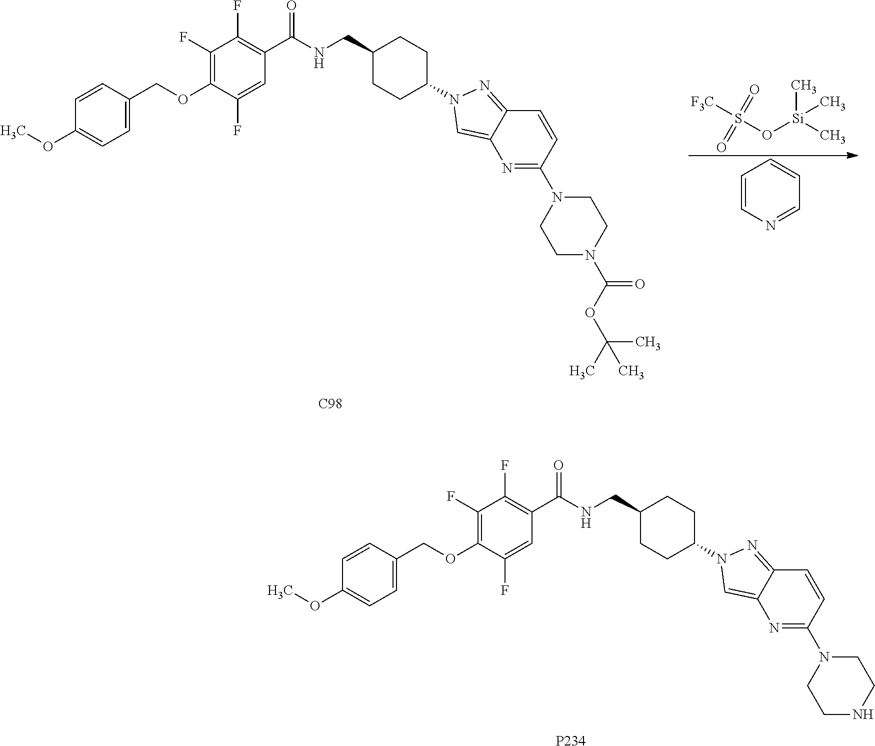

Step 1. Synthesis of tert-butyl 4-(6-methyl-5-nitropyridin-2-yl)piperazine-1-carboxylate (C95): A solution of 6-chloro-2-methyl-3-nitropyridine (2.50 g, 14.5 mmol) and tert-butyl piperazine-1-carboxylate (2.70 g, 14.5 mmol) in triethylamine (50 mL) was stirred at 80° C. for 18 hours. The reaction mixture was then concentrated in vacuo, and the residue was treated with water (100 mL); the resulting mixture was filtered, and the filter cake was washed with water (100 mL) to afford C95 as a yellow solid. Yield: 4.50 g, 14.0 mmol, 96%. LCMS m/z 323.1 [M+H]⁺. ¹H NMR (400 MHz, DMSO-d₆) δ 8.22 (d, J=9.4 Hz, 1H), 6.82 (d, J=9.4 Hz, 1H), 3.78-3.70 (m, 4H), 3.47-3.39 (m, 4H), 2.67 (s, 3H), 1.42 (s, 9H).

Step 2. Synthesis of tert-butyl 4-{6-[(E)-2-(dimethylamino)ethenyl]-5-nitropyridin-2-yl}piperazine-1-carboxylate (C96): A solution of C95 (4.50 g, 14.0 mmol) in a mixture of N,N-dimethylformamide dimethyl acetal (15 mL) and N,N-dimethylformamide (15 mL) was stirred at 110° C. for 16 hours, whereupon water (100 mL) was added. The resulting mixture was filtered, and the filter cake was washed with water (100 mL), providing C96 as a brown solid. Yield: 5.00 g, 13.2 mmol, 94%. LCMS m/z 378.2 [M+H]⁺. ¹H NMR (400 MHz, chloroform-d) δ 8.21 (d, J=9.4 Hz, 1H), 7.95 (d, J=12.4 Hz, 1H), 6.50 (d, J=12.5 Hz, 1H), 6.17 (d, J=9.5 Hz, 1H), 3.73-3.66 (m, 4H), 3.57-3.50 (m, 4H), 3.03 (s, 6H), 1.48 (s, 9H).

Step 3. Synthesis of tert-butyl 4-(6-formyl-5-nitropyridin-2-yl)piperazine-1-carboxylate (C97): Sodium periodate (8.58 g, 40.1 mmol) was added portion-wise over 5 minutes to a solution of C96 (5.00 g, 13.2 mmol) in a mixture of tetrahydrofuran (70 mL) and water (70 mL). After the reaction mixture had been stirred at 25° C. for 4 days, it was filtered, and the collected solids were rinsed with ethyl acetate (150 mL). The combined filtrates were washed sequentially with saturated aqueous sodium bicarbonate solution (150 mL) and aqueous sodium sulfite solution (150 mL), whereupon the combined aqueous layers were extracted with ethyl acetate (3×100 mL). The combined organic layers were washed with saturated aqueous sodium chloride solution, dried over sodium sulfate, filtered, and concentrated in vacuo. Chromatography on silica gel (Eluent: 2:1 petroleum ether/ethyl acetate) provided C97 as a yellow solid. Yield: 2.30 g, 6.84 mmol, 52%. LCMS m/z 281.1 [(M-2-methylprop-1-ene)+H]⁺. ¹H NMR (400 MHz, DMSO-d₆) δ 10.22 (s, 1H), 8.27 (d, J=9.5 Hz, 1H), 7.08 (d, J=9.6 Hz, 1H), 3.82-3.74 (m, 4H), 3.49-3.42 (m, 4H), 1.42 (s, 9H).

Step 4. Synthesis of tert-butyl 4-{2-[(1r,4r)-4-({2,3,5-trifluoro-4-[(4-methoxyphenyl)methoxy]benzamido}methyl)cyclohexyl]-2H-pyrazolo[4,3-b]pyridin-5-yl}piperazine-1-carboxylate (C98): A solution of P233 (600 mg, 1.42 mmol) and C97 (478 mg, 1.42 mmol) in propan-2-ol (50 mL) was stirred at 85° C. for 16 hours, whereupon it was cooled to 25° C. After addition of tributylphosphine (1.15 g, 5.68 mmol), the reaction mixture was stirred at 85° C. for 16 hours, then concentrated in vacuo. Purification of the residue using silica gel chromatography (Gradient: 0% to 100% ethyl acetate in petroleum ether, then 0% to 20% methanol in dichloromethane) afforded C98 as a yellow oil. Yield: 220 mg, 0.310 mmol, 22%. LCMS m/z 709.3 [M+H]⁺.

Step 5. Synthesis of 2,3,5-trifluoro-4-[(4-methoxyphenyl)methoxy]-N-({(1r,4r)-4-[5-(piperazin-1-yl)-2H-pyrazolo[4,3-b]pyridin-2-yl]cyclohexyl}methyl)benzamide (P234): Trimethylsilyl trifluoromethanesulfonate (276 mg, 1.24 mmol) was added drop-wise to a 0° C. mixture of C98 (220 mg, 0.310 mmol) and pyridine (196 mg, 2.48 mmol) in dichloromethane (20 mL). The reaction mixture was stirred at 25° C. for 16 hours, whereupon it was treated with aqueous sodium carbonate solution (5 mL) and aqueous sodium bicarbonate solution (5 mL). The resulting mixture was extracted with dichloromethane (2×30 mL), and the combined organic layers were concentrated in vacuo; silica gel chromatography (Gradient: 0% to 100% ethyl acetate in petroleum ether, then 0% to 20% methanol in dichloromethane) provided P234 as a yellow solid. Yield: 150 mg, 0.246 mmol, 79%. LCMS m/z 609.3 [M+H]⁺.

### Preparation P235

### (1r,4r)-4-({2,3,5-Trifluoro-4-[(4-methoxyphenyl)methoxy]benzamido}methyl)cyclohexane-1-carboxylic acid (P235)

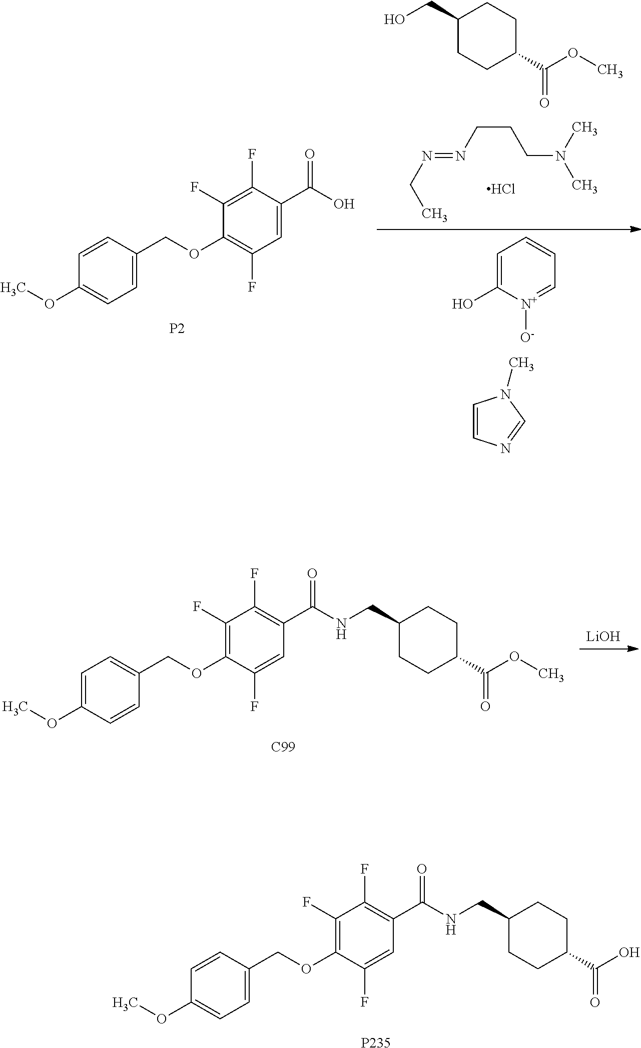

Step 1. Synthesis of methyl (1r,4r)-4-({2,3,5-trifluoro-4-[(4-methoxyphenyl)methoxy]benzamido}methyl)cyclohexane-1-carboxylate (C99): To a solution of P2 (1.66 g, 5.32 mmol), methyl (1r,4r)-4-(aminomethyl)cyclohexane-1-carboxylate (1.00 g, 5.84 mmol), 1-methyl-1H-imidazole (1.27 mL, 15.9 mmol), and 2-hydroxypyridine 1-oxide (590 mg, 5.31 mmol) in N,N-dimethylformamide (15 mL) was added 1-[3-(dimethylamino)propyl]-3-ethylcarbodiimide hydrochloride (98%, 1.14 g, 5.83 mmol), whereupon the reaction mixture was gradually heated to 60° C. After it had been stirred overnight at 60° C., the reaction mixture was cooled to room temperature, poured into cold water (100 mL), and partitioned between saturated aqueous sodium chloride solution (50 mL) and ethyl acetate (60 mL). The aqueous layer was extracted with ethyl acetate (2×60 mL), and the combined organic layers were dried over sodium sulfate, filtered, and concentrated in vacuo. Silica gel chromatography (Gradient: 0% to 100% ethyl acetate in heptane) afforded C99 as a white solid. Yield: 2.28 g, 4.90 mmol, 92%. LCMS m/z 466.4 [M+H]⁺. ¹H NMR (400 MHz, DMSO-d₆) δ 8.40 (br t, J=6 Hz, 1H), 7.35 (d, J=8.6 Hz, 2H), 7.35-7.29 (m, 1H), 6.94 (d, J=8.7 Hz, 2H), 5.21 (s, 2H), 3.75 (s, 3H), 3.58 (s, 3H), 3.07 (t, J=6.3 Hz, 2H), 2.25 (tt, J=12.2, 3.6 Hz, 1H), 1.95-1.86 (m, 2H), 1.81-1.72 (m, 2H), 1.54-1.40 (m, 1H), 1.36-1.21 (m, 2H), 1.04-0.90 (m, 2H).

Step 2. Synthesis of (1r,4r)-4-({2,3,5-trifluoro-4-[(4-methoxyphenyl)methoxy]benzamido}methyl)cyclohexane-1-carboxylic acid (P235): A 50° C. solution of C99 (2.28 g, 4.90 mmol) in a mixture of tetrahydrofuran (10 mL), water (10 mL), and methanol (5.0 mL) was treated with lithium hydroxide (3.52 g, 147 mmol). After four hours at 50° C., the reaction mixture was allowed to cool to room temperature, whereupon the organic solvents were removed via concentration in vacuo. The aqueous residue was diluted with water (100 mL) and saturated aqueous sodium chloride solution (50 mL), and subsequently adjusted to pH 2 by addition of concentrated hydrochloride acid. The resulting mixture was extracted with ethyl acetate (3×100 mL); the combined organic layers were washed with saturated aqueous sodium chloride solution, dried over sodium sulfate, filtered, and concentrated under reduced pressure. This crude product was dissolved in methanol, treated with silica gel, and concentrated in vacuo to allow dry-loading of the silica gel column; silica gel chromatography [Gradient: 0% to 100% (1% acetic acid in ethyl acetate) in heptane] afforded P235 as a solid. Yield: 1.40 g, 3.10 mmol, 63%. LCMS m/z 452.3 [M+H]⁺. ¹H NMR (400 MHz, DMSO-d₆) δ 11.98 (s, 1H), 8.39 (br t, J=6 Hz, 1H), 7.35-7.29 (m, 1H), 7.34 (d, J=8.6 Hz, 2H), 6.93 (d, J=8.6 Hz, 2H), 5.21 (s, 2H), 3.75 (s, 3H), 3.07 (t, J=6.3 Hz, 2H), 2.12 (tt, J=12.1, 3.5 Hz, 1H), 1.94-1.84 (m, 2H), 1.81-1.71 (m, 2H), 1.52-1.39 (m, 1H), 1.33-1.19 (m, 2H), 1.02-0.88 (m, 2H).

### Preparation P236

### 2,3,5-Trifluoro-4-[(4-methoxyphenyl)methoxy]-N-{[(1r,4r)-4-{5-[2-(piperazin-1-yl)pyrimidin-4-yl]-1,2,4-oxadiazol-3-yl}cyclohexyl]methyl}benzamide (P236)

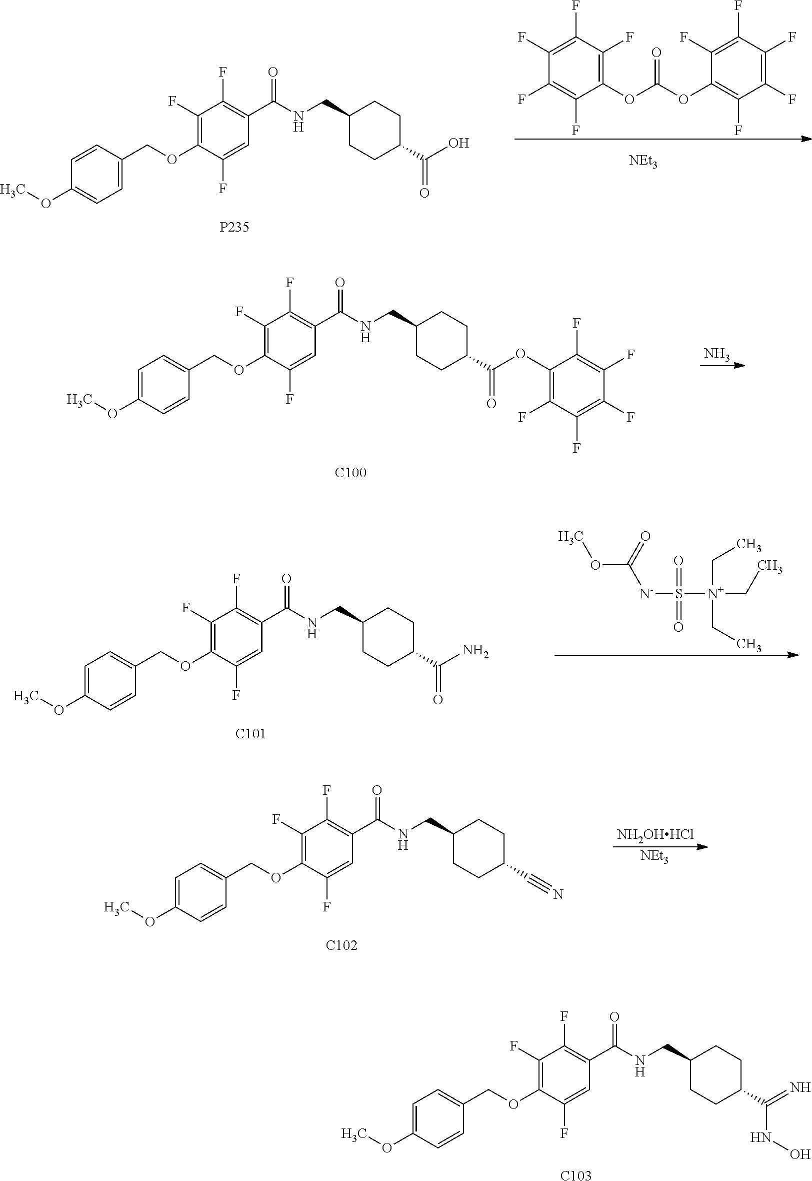

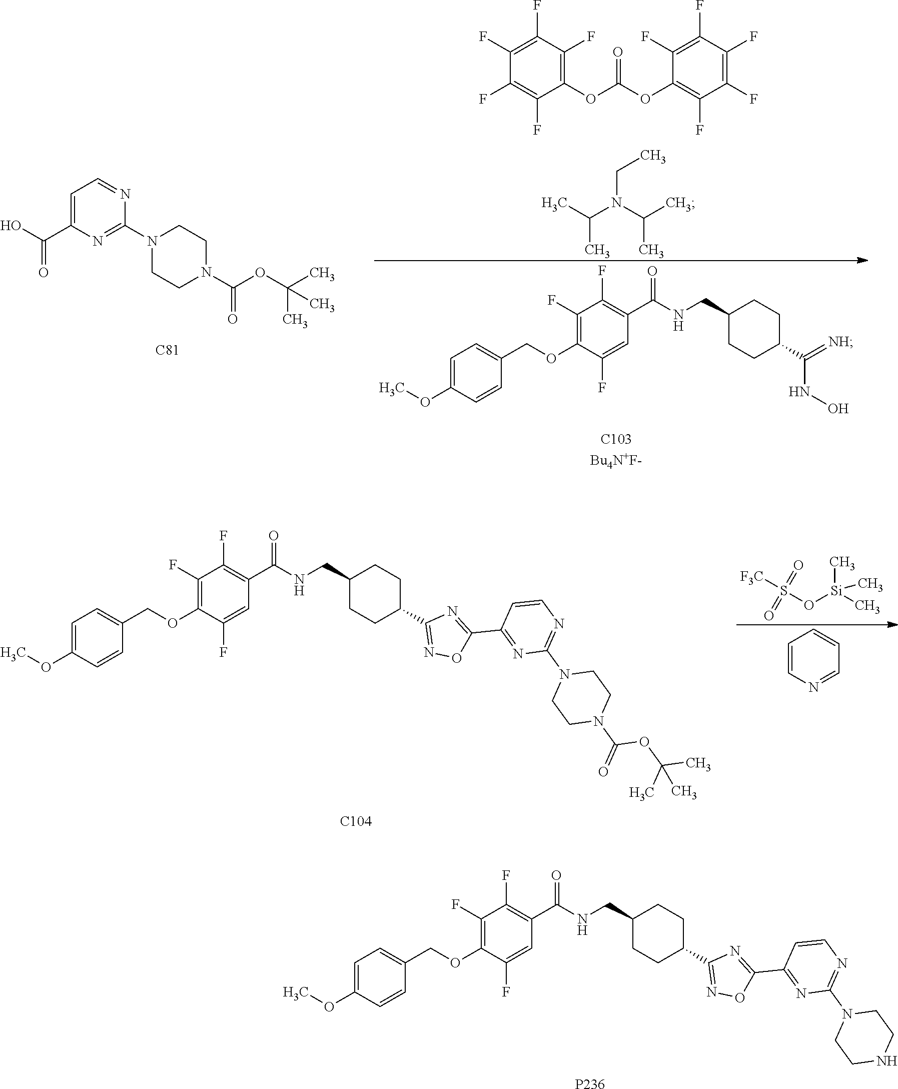

Step 1. Synthesis of pentafluorophenyl (1r,4r)-4-({2,3,5-trifluoro-4-[(4-methoxyphenyl)methoxy]benzamido}methyl)cyclohexane-1-carboxylate (C100): Triethylamine (0.454 mL, 330 mg, 3.26 mmol) was added to a solution of P235 (646 mg, 1.43 mmol) and bis(pentafluorophenyl) carbonate (98%, 668 mg, 1.66 mmol) in acetonitrile (5 mL). The reaction was allowed to stir at room temperature for 30 minutes, whereupon it was diluted with cold acetonitrile and filtered. The filter cake was washed with acetonitrile and with water; the combined filtrate produced additional precipitate, which was also collected via filtration. The washing and filtration were repeated with this second filter cake, and the three lots of solid were combined to afford C100 as a white solid. Yield: 758 mg, 1.23 mmol, 86%. LCMS m/z 618.3 [M+H]⁺. ¹H NMR (400 MHz, DMSO-d₆) δ 8.44 (br t, J=6 Hz, 1H), 7.36-7.31 (m, 1H), 7.35 (d, J=8.7 Hz, 2H), 6.94 (d, J=8.7 Hz, 2H), 5.21 (s, 2H), 3.75 (s, 3H), 3.11 (t, J=6.3 Hz, 2H), 2.78 (tt, J=12.0, 3.5 Hz, 1H), 2.15-2.06 (m, 2H), 1.88-1.78 (m, 2H), 1.62-1.39 (m, 3H), 1.15-1.01 (m, 2H).

Step 2. Synthesis of N-{[(1r,4r)-4-carbamoylcyclohexyl]methyl}-2,3,5-trifluoro-4-[(4-methoxyphenyl)methoxy]benzamide (C101): A solution of ammonia in tetrahydrofuran (0.40 M; 6.0 mL, 2.4 mmol) was added to a solution of C100 (350 mg, 0.567 mmol) in tetrahydrofuran (2.0 mL), and the reaction mixture was allowed to stir at room temperature for 3 hours. Partial concentration at 25° C. and 300 to 100 millibar produced a precipitate; after addition of methyl tert-butyl ether, the mixture was stirred for 1 hour. Filtration provided C101 as a white solid. Yield: 210 mg, 0.466 mmol, 82%. LCMS m/z 451.4 [M+H]⁺. ¹H NMR (400 MHz, DMSO-d₆) δ 8.39 (br t, J=6 Hz, 1H), 7.35 (d, J=8.6 Hz, 2H), 7.35-7.29 (m, 1H), 7.16 (br s, 1H), 6.94 (d, J=8.7 Hz, 2H), 6.64 (br s, 1H), 5.21 (s, 2H), 3.75 (s, 3H), 3.07 (t, J=6.3 Hz, 2H), 2.07-1.95 (m, 1H), 1.80-1.70 (m, 4H), 1.52-1.38 (m, 1H), 1.35-1.21 (m, 2H), 0.99-0.85 (m, 2H).

Step 3. Synthesis of N-{[(1r,4r)-4-cyanocyclohexyl]methyl}-2,3,5-trifluoro-4-[(4-methoxyphenyl)methoxy]benzamide (C102): To a suspension of C101 (524 mg, 1.16 mmol) in ethyl acetate (11.6 mL) was added methyl N-(triethylammoniosulfonyl)carbamate, inner salt (Burgess reagent; 704 mg, 2.95 mmol). After the reaction mixture had been stirred at room temperature for 4 hours, it was concentrated in vacuo and purified using silica gel chromatography (Gradient: 5% to 100% ethyl acetate in heptane) to afford C102 as a white solid. Yield: 385 mg, 0.890 mmol, 77%. LCMS m/z 433.3 [M+H]⁺. ¹H NMR (400 MHz, DMSO-d₆) δ 8.42 (br t, J=6 Hz, 1H), 7.35 (d, J=8.7 Hz, 2H), 7.35-7.29 (m, 1H), 6.94 (d, J=8.7 Hz, 2H), 5.21 (s, 2H), 3.75 (s, 3H), 3.07 (t, J=6.3 Hz, 2H), 2.62 (tt, J=11.9, 3.7 Hz, 1H), 2.05-1.95 (m, 2H), 1.78-1.68 (m, 2H), 1.58-1.38 (m, 3H), 1.04-0.90 (m, 2H).

Step 4. Synthesis of 2,3,5-trifluoro-N-{[(1r,4r)-4-(N-hydroxycarbamimidoyl)cyclohexyl]methyl}-4-[(4-methoxyphenyl)methoxy]benzamide (C103): Hydroxylamine hydrochloride (309 mg, 4.45 mmol) and triethylamine (0.622 mL, 4.46 mmol) were added to a suspension of C102 (385 mg, 0.890 mmol) in methanol (5.6 mL). The reaction mixture was heated at 55° C. for 24 hours, whereupon it was cooled to room temperature and concentrated under reduced pressure. After the residue had been dissolved in ethyl acetate, it was washed with saturated aqueous sodium chloride solution and concentrated in vacuo, affording C103 as a light-pink solid (544 mg). This material was impure; most of it was progressed directly to the following step. LCMS m/z 466.3 [M+H]⁺.

Step 5. Synthesis of tert-butyl 4-(4-{3-[(1r,4r)-4-({2,3,5-trifluoro-4-[(4-methoxyphenyl)methoxy]benzamido}methyl)cyclohexyl]-1,2,4-oxadiazol-5-yl}pyrimidin-2-yl)piperazine-1-carboxylate (C104): N,N-Diisopropylethylamine (0.240 mL, 1.38 mmol) was added drop-wise to a solution of C81 (156 mg, 0.506 mmol) and bis(pentafluorophenyl) carbonate (98%, 194 mg, 0.482 mmol) in tetrahydrofuran (2.3 mL). After the reaction mixture had been stirred at room temperature for 30 minutes, C103 (from the previous step, estimated purity of 40%; 535 mg, 0.46 mmol) was added and stirring was continued at room temperature for 1 hour. A solution of tetrabutylammonium fluoride in tetrahydrofuran (1.0 M; 2.30 mL, 2.30 mmol) was then added and the reaction mixture was heated at 50° C. overnight, whereupon it was cooled to room temperature, treated with a small amount of aqueous sodium bicarbonate solution, diluted with water, and extracted three times with ethyl acetate. The combined organic layers were dried over magnesium sulfate, filtered, and concentrated in vacuo; silica gel chromatography (Gradient: 5% to 80% ethyl acetate in heptane) provided C104 as a yellow solid. Yield: 89.0 mg, 0.121 mmol, 26%. LCMS m/z 682.5 [(M-2-methylprop-1-ene)+H]⁺.

Step 6. Synthesis of 2,3,5-trifluoro-4-[(4-methoxyphenyl)methoxy]-N-{[(1r,4r)-4-{5-[2-(piperazin-1-yl)pyrimidin-4-yl]-1,2,4-oxadiazol-3-yl}cyclohexyl]methyl}benzamide (P236): Trimethylsilyl trifluoromethanesulfonate (71.6 μL, 0.396 mmol) was added drop-wise to a −15° C. (methanol/ice bath slurry) solution of C104 (73 mg, 99 μmol) and pyridine (64.0 μL, 0.791 mmol) in dichloromethane (3.2 mL). The reaction mixture was allowed to stir overnight in the methanol/ice bath, at which point the bath had warmed to 12° C. After the reaction mixture had been cooled to 0° C., aqueous sodium bicarbonate solution (10 mL) was added slowly, and the resulting mixture was stirred for 10 minutes. The pH of the aqueous layer was adjusted from 7-8 to pH 10, whereupon it was extracted three times with dichloromethane. The combined organic layers were washed sequentially with saturated aqueous sodium bicarbonate solution and saturated aqueous sodium chloride solution, dried over magnesium sulfate, filtered, and concentrated in vacuo to afford P236 as a yellow solid. Yield: 63 mg, 99 μmol, quantitative. LCMS m/z 638.4 [M+H]⁺. ¹H NMR (400 MHz, DMSO-d₆) δ 8.64 (d, J=4.8 Hz, 1H), 8.45 (br t, J=6 Hz, 1H), 7.40-7.30 (m, 3H), 7.25 (d, J=4.8 Hz, 1H), 6.94 (d, J=8.6 Hz, 2H), 5.22 (s, 2H), 3.77-3.70 (m, 4H), 3.75 (s, 3H), 3.14 (t, J=6.3 Hz, 2H), 2.85 (tt, J=12.0, 3.4 Hz, 1H), 2.80-2.72 (m, 4H), 2.14-2.03 (m, 2H), 1.92-1.82 (m, 2H), 1.66-1.44 (m, 3H), 1.20-1.07 (m, 2H).

### Preparation P237

### 1-{4-[2-(2-Chloropyrimidin-4-yl)-1,3-oxazol-5-yl]bicyclo[2.2.2]octan-1-yl}methanamine (P237)

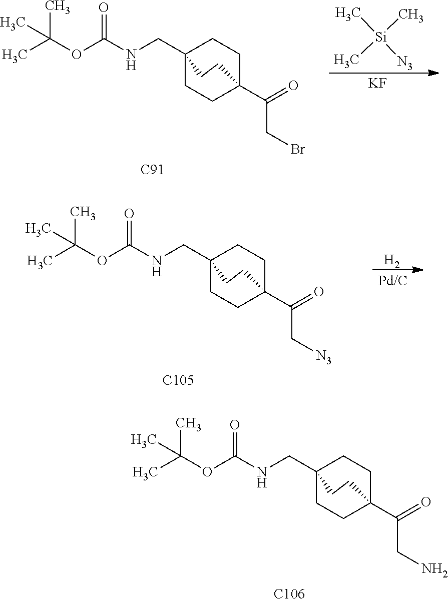

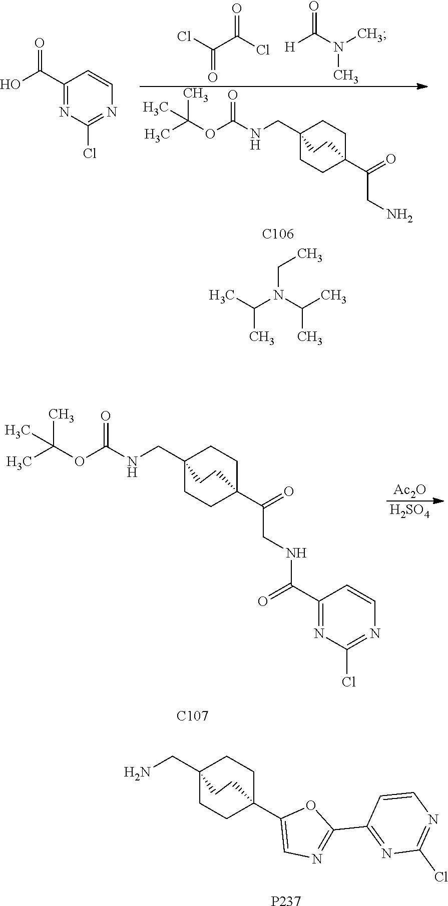

Step 1. Synthesis of tert-butyl {[4-(azidoacetyl)bicyclo[2.2.2]octan-1-yl]methyl}carbamate (C105): To a stirred solution of C91 (650 mg, 1.80 mol) in ethanol (5 mL) were added azido(trimethyl)silane (203 mg, 1.76 mmol) and potassium fluoride (6.81 mg, 0.117 mmol). After the reaction mixture had been stirred at 20° C. for 40 hours, it was concentrated in vacuo to provide C105 as an oil. Yield: 590 mg, 1.8 mmol, quantitative. LCMS m/z 345.2 [M+Na⁺].

Step 2. Synthesis of tert-butyl [(4-glycylbicyclo[2.2.2]octan-1-yl)methyl]carbamate (C106): A mixture of C105 (635 mg, 1.97 mmol) and palladium on carbon (79 mg) in methanol (5 mL) was hydrogenated for 3 hours at 20° C. After filtration of the reaction mixture through diatomaceous earth and rinsing of the filter cake with dichloromethane, the combined filtrates were concentrated in vacuo, affording C106 as a brown oil. Yield: 535 mg, 1.80 mmol, 91%. LCMS m/z 297.3 [M+H]⁺.

Step 3. Synthesis of tert-butyl ({4-[N-(2-chloropyrimidine-4-carbonyl)glycyl]bicyclo[2.2.2]octan-1-yl}methyl)carbamate (C107): N,N-Dimethylformamide (32.3 mg, 0.442 mmol) was added to a solution of 2-chloropyrimidine-4-carboxylic acid (70.0 mg, 0.442 mmol) and oxalyl chloride (67.3 mg, 0.530 mmol) in dichloromethane (10 mL). After this mixture had been stirred at 25° C. for 1 hour, C106 (131 mg, 0.442 mmol) and N,N-diisopropylethylamine (114 mg, 0.882 mmol) were added, and the reaction mixture was stirred at 25° C. overnight. It was then concentrated in vacuo and purified via chromatography on silica gel (Gradient: 0% to 10% methanol in dichloromethane), providing C107 as a yellow solid. Yield: 165 mg, 0.378 mmol, 86%. LCMS m/z 437.1 (chlorine isotope pattern observed) [M+H]⁺. ¹H NMR (400 MHz, DMSO-d₆) δ 9.03 (d, J=5.0 Hz, 1H), 8.92 (br t, J=5.7 Hz, 1H), 8.00 (d, J=4.9 Hz, 1H), 6.75 (br t, J=6.4 Hz, 1H), 4.26 (d, J=5.7 Hz, 2H), 2.70 (d, J=6.4 Hz, 2H), 1.73-1.63 (m, 6H), 1.40-1.29 (m, 15H).

Step 4. Synthesis of 1-{4-[2-(2-chloropyrimidin-4-yl)-1,3-oxazol-5-yl]bicyclo[2.2.2]octan-1-yl}methanamine (P237): To a 0° C. solution of C107 (100 mg, 0.229 mmol) in acetic anhydride (1.5 mL) was added concentrated sulfuric acid (1.5 mL). After the reaction mixture had been stirred at 25° C. for 2 hours, it was added to ice water (20 mL), and the pH of the resulting mixture was adjusted to 8 by addition of aqueous sodium carbonate solution. It was then extracted with ethyl acetate (30 mL), and the combined organic layers were washed twice with saturated aqueous sodium chloride solution, dried over sodium sulfate, filtered, and concentrated in vacuo to afford P237 as a white solid. Yield: 56.0 mg, 0.176 mmol, 77%. LCMS m/z 319.1 (chlorine isotope pattern observed) [M+H]⁺.

### Preparation P238

### 4-({[2-(2,6-Dioxopiperidin-3-yl)-1-oxo-2,3-dihydro-1H-isoindol-4-yl]oxy}methyl)benzaldehyde

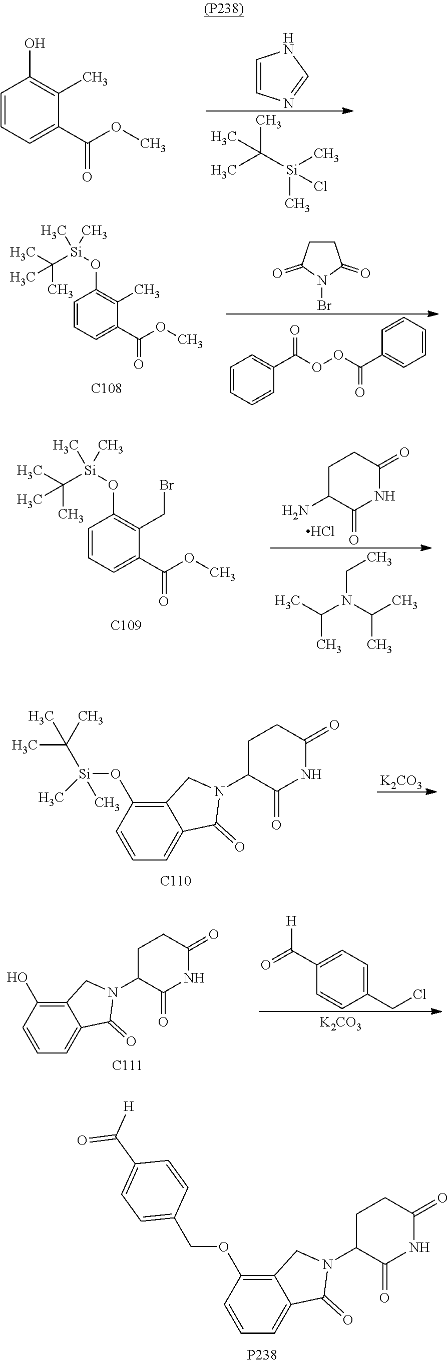

Step 1. Synthesis of methyl 3-{[tert-butyl(dimethyl)silyl]oxy}-2-methylbenzoate (C108): 1H-Imidazole (113 g, 1.66 mmol) was added portion-wise to a solution of methyl 3-hydroxy-2-methylbenzoate (110 g, 662 mmol) in N,N-dimethylformamide (1.0 L), whereupon the mixture was cooled to 0° C. in a bath containing ice and saturated aqueous sodium chloride solution and treated portion-wise with tert-butyl(dimethyl)silyl chloride (150 g, 995 mmol). After completion of the addition, the reaction mixture was allowed to warm to room temperature (25° C.) and stir for 40 hours; it was then poured into ice water (600 mL) and extracted with ethyl acetate (3×600 mL). The combined ethyl acetate layers were washed sequentially with water (600 mL) and saturated aqueous sodium chloride solution (3×600 mL), dried over sodium sulfate, filtered, and concentrated in vacuo. Silica gel chromatography (Gradient: 0% to 10% ethyl acetate in petroleum ether) afforded C108 as a yellow oil. Yield: 182 g, 649 mmol, 98%. ¹H NMR (400 MHz, chloroform-d) δ 7.42 (dd, J=7.8, 1.3 Hz, 1H), 7.09 (t, J=7.9 Hz, 1H), 6.93 (dd, J=8.0, 1.3 Hz, 1H), 3.88 (s, 3H), 2.41 (s, 3H), 1.02 (s, 9H), 0.21 (s, 6H).

Step 2. Synthesis of methyl 2-(bromomethyl)-3-{[tert-butyl(dimethyl)silyl]oxy}benzoate (C109): N-Bromosuccinimide (97.7 g, 549 mmol) and benzoyl peroxide (9.67 g, 39.9 mmol) were added to a solution of C108 (140 g, 499 mmol) in ethyl acetate (900 mL). After the reaction mixture had been heated at reflux (70° C.) for 18 hours, it was cooled to room temperature, diluted with ethyl acetate (800 mL), washed sequentially with saturated aqueous sodium sulfite solution (2×800 mL) and saturated aqueous sodium chloride solution (800 mL), dried over sodium sulfate, filtered, and concentrated in vacuo to afford C109 as a light-brown oil (180 g). The bulk of this material was progressed to the following step. ¹H NMR (400 MHz, chloroform-d) δ 7.52 (br d, J=7.8 Hz, 1H), 7.23 (t, J=8.0 Hz, 1H), 7.00 (br d, J=8.1 Hz, 1H), 5.02 (s, 2H), 3.93 (s, 3H), 1.07 (s, 9H), 0.31 (s, 6H).

Step 3. Synthesis of 3-(4-{[tert-butyl(dimethyl)silyl]oxy}-1-oxo-1,3-dihydro-2H-isoindol-2-yl)piperidine-2,6-dione (C110): 3-Aminopiperidine-2,6-dione, hydrochloride salt (95.3 g, 579 mmol) and N,N-diisopropylethylamine (197 mL, 1.13 mol) were added to a solution of C109 (from the previous step; 160 g, 5444 mmol) in acetonitrile (1.5 L), whereupon the reaction mixture was heated at 50° C. for 16 hours, then concentrated in vacuo. The residue was partitioned between water (200 mL) and ethyl acetate (200 mL), subjected to ultrasonic mixing for 3 minutes, and filtered. The filter cake was washed with water (2×100 mL) and ethyl acetate (2×100 mL) to provide C110 as a light-blue solid. Yield: 125 g, 334 mmol, 75% over 2 steps. LCMS m/z 374.8 [M+H]⁺.

Step 4. Synthesis of 3-(4-hydroxy-1-oxo-1,3-dihydro-2H-isoindol-2-yl)piperidine-2,6-dione (C111): Potassium carbonate (12.9 g, 93.3 mmol) was added to a solution of C110 (70.0 g, 187 mmol) in a mixture of N,N-dimethylformamide (700 mL) and water (100 mL), whereupon the reaction mixture was stirred at 25° C. for 40 minutes. It was then acidified to pH 4 by addition of 1 M hydrochloric acid, and concentrated in vacuo to remove N,N-dimethylformamide and water. The residue was partitioned between water (200 mL) and ethyl acetate (200 mL), stirred at 15° C. for 1 hour, and filtered; the filter cake was washed with ethyl acetate (3×100 mL) to afford C111 as a light-blue solid. Yield: 37.0 g, 142 mmol, 76%. LCMS m/z 261.0 [M+H]⁺. ¹H NMR (400 MHz, DMSO-d₆) δ 10.99 (br s, 1H), 10.13 br (s, 1H), 7.33 (t, J=7.7 Hz, 1H), 7.18 (d, J=7.4 Hz, 1H), 7.02 (d, J=7.9 Hz, 1H), 5.10 (dd, J=13.3, 5.1 Hz, 1H), 4.25 (AB quartet, J_{AB}=17.1 Hz, Δv_{AB}=55.4 Hz, 2H), 2.97-2.85 (m, 1H), 2.64-2.54 (m, 1H), 2.48-2.34 (m, 1H), 2.05-1.94 (m, 1H).

Step 5. Synthesis of 4-({[2-(2,6-dioxopiperidin-3-yl)-1-oxo-2,3-dihydro-1H-isoindol-4-yl]oxy}methyl)benzaldehyde (P238): Potassium carbonate (15.9 g, 115 mmol) and 4-(chloromethyl)benzaldehyde (16.3 g, 105 mmol) were added to a solution of C111 (25.0 g, 96.1 mmol) in acetonitrile (600 mL). After the reaction mixture had been heated at 60° C. for 16 hours, potassium carbonate (2.66 g, 19.2 mmol) and 4-(chloromethyl)benzaldehyde (4.46 g, 28.8 mmol) were again added, and heating was continued at 70° C. for an additional 36 hours. The reaction mixture was concentrated in vacuo, partitioned between water (250 mL) and ethyl acetate (200 mL), and stirred at 15° C. for 1 hour. The resulting mixture was filtered; the filter cake was washed with water (2×50 mL), then treated with dichloromethane (200 mL) and stirred at 15° C. for 20 minutes. A second filtration provided a solid, which was washed with dichloromethane (2×30 mL) to afford P238 as a light-blue solid. Yield: 26.0 g, 68.7 mmol, 71%. LCMS m/z 401.1 [M+Na⁺]. ¹H NMR (400 MHz, DMSO-d₆) δ 10.99 (br s, 1H), 10.02 (s, 1H), 7.94 (d, J=8.1 Hz, 2H), 7.71 (d, J=8.0 Hz, 2H), 7.49 (t, J=7.8 Hz, 1H), 7.34 (d, J=7.5 Hz, 1H), 7.31 (d, J=8.1 Hz, 1H), 5.39 (s, 2H), 5.12 (dd, J=13.3, 5.1 Hz, 1H), 4.39 (AB quartet, J_{AB}=17.5 Hz, Δv_{AB}=63.7 Hz, 2H), 2.91 (ddd, J=17.1, 13.6, 5.3 Hz, 1H), 2.63-2.54 (m, 1H), 2.5-2.38 (m, 1H, assumed; partially obscured by solvent peak), 2.04-1.94 (m, 1H).

### Preparation P239

### Pentafluorophenyl 3-(2,4-dioxo-1,3-diazinan-1-yl)-4-methyl benzoate (P239)

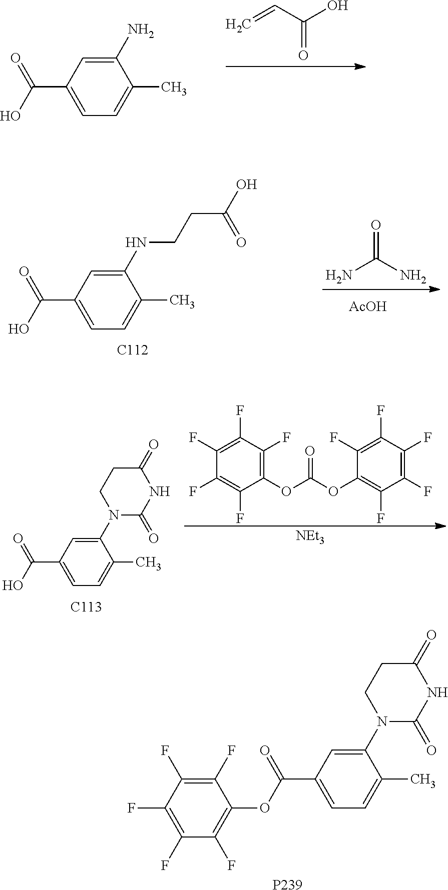

Step 1. Synthesis of 3-[(2-carboxyethyl)amino]-4-methylbenzoic acid (C112): A solution of 3-amino-4-methylbenzoic acid (50 g, 330 mmol) in prop-2-enoic acid (191 g, 2.65 mol) was stirred at 100° C. for 3 hours, providing C112. The reaction mixture was progressed directly to the following step. LCMS m/z 223.9 [M+H]⁺.

Step 2. Synthesis of 3-(2,4-dioxo-1,3-diazinan-1-yl)-4-methylbenzoic acid (C113): A mixture of C112 (from the previous step, still in prop-2-enoic acid; 5330 mmol) and urea (129 g, 2.15 mol) in acetic acid (400 mL) was stirred for 16 hours at 100° C., whereupon the reaction mixture was cooled to 20° C. and diluted with diluted with water (700 mL). After the resulting mixture had been stirred for 20 minutes at 20° C., it was filtered. The filter cake was dried under reduced pressure at 60° C. and then lyophilized, affording C113 as a white solid. Yield: 54.0 g, 218 mmol, 66% over 2 steps. LCMS m/z 248.9 [M+H]⁺. ¹H NMR (400 MHz, DMSO-d₆) δ 12.96 (br s, 1H), 10.40 (s, 1H), 7.82 (br d, half of AB quartet, J=1.7 Hz, 1H), 7.80 (br dd, component of ABX system, J=7.9, 1.8 Hz, 1H), 7.42 (d, J=7.9 Hz, 1H), 3.83 (ddd, J=12.2, 9.5, 5.4 Hz, 1H), 3.59-3.48 (m, 1H), 2.84-2.65 (m, 2H), 2.25 (s, 3H).

Step 3. Synthesis of pentafluorophenyl 3-(2,4-dioxo-1,3-diazinan-1-yl)-4-methylbenzoate (P239): Triethylamine (5.90 mL, 42.3 mmol) was added over approximately 1 minute to a stirring suspension of C113 (4.99 g, 20.1 mmol) and bis(pentafluorophenyl) carbonate (97%, 8.49 g, 20.9 mmol) in acetonitrile (32 mL). After the reaction mixture had been stirred at room temperature for 1 hour, the solid was collected via filtration and washed with cold acetonitrile (20 mL), providing P239 as a white solid. Yield: 7.99 g, 19.3 mmol, 96%. LCMS m/z 415.3 [M+H]⁺. ¹H NMR (400 MHz, chloroform-d) δ 8.10 (dd, J=8.0, 1.8 Hz, 1H), 8.02 (d, J=1.8 Hz, 1H), 7.89 (br s, 1H), 7.49 (d, J=8.1 Hz, 1H), 3.98-3.88 (m, 1H), 3.73-3.64 (m, 1H), 2.89 (t, J=6.7 Hz, 2H), 2.40 (s, 3H).

### Preparation P240

### [1-(2,6-Dioxopiperidin-3-yl)-3-methyl-2-oxo-2,3-dihydro-1H-benzimidazol-5-yl]acetaldehyde (P240)

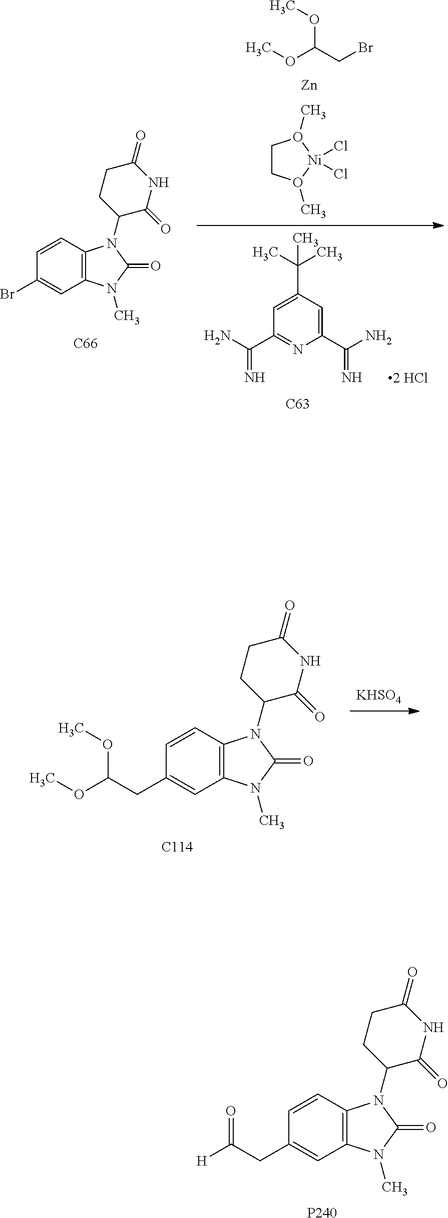

Step 1. Synthesis of 3-[5-(2,2-dimethoxyethyl)-3-methyl-2-oxo-2,3-dihydro-1H-benzimidazol-1-yl]piperidine-2,6-dione (C114): N,N-Dimethylacetamide was degassed under vacuum and then purged with nitrogen; this evacuation-purge cycle was carried out a total of three times. 2-Bromo-1,1-dimethoxyethane was degassed by bubbling nitrogen through it for 2 minutes.

A mixture of C66 (10.1 g, 29.9 mmol), nickel(II) chloride, ethylene glycol dimethyl ether complex (659 mg, 3.00 mmol), C63 (872 mg, 2.98 mmol), and zinc flakes (325 mesh; 3.92 g, 60.0 mmol) was evacuated under vacuum, then thoroughly flushed twice with nitrogen. Degassed N,N-dimethylacetamide (32 mL) was added, followed by addition of degassed 2-bromo-1,1-dimethoxyethane (97%, 10.5 mL, 86.2 mmol), whereupon the reaction mixture was heated at 65° C. for 24 hours. After the reaction mixture had cooled to room temperature, it was poured into ethyl acetate (170 mL), stirred for 10 minutes, and filtered through diatomaceous earth. The filter cake was rinsed with ethyl acetate (320 mL), and the combined filtrates were filtered through a small pad of diatomaceous earth. This filter cake was also rinsed with ethyl acetate (100 mL), and the combined filtrates were washed with aqueous lithium chloride solution (20%, 300 mL). The aqueous layer was extracted with ethyl acetate (3×200 mL), and all the ethyl acetate extracts were combined, dried over magnesium sulfate, filtered, and concentrated in vacuo. To the resulting solid was added dichloromethane (20 mL), followed by ethyl acetate (80 mL), and the suspension was stirred at room temperature for 1 hour; filtration afforded C114 as an off-white solid. Yield: 7.08 g, 20.4 mmol, 68%. LCMS m/z 348.3 [M+H]⁺. ¹H NMR (400 MHz, chloroform-d) δ 8.25 (br s, 1H), 6.96-6.90 (m, 2H), 6.72 (d, J=7.9 Hz, 1H), 5.21 (dd, J=12.6, 5.3 Hz, 1H), 4.53 (t, J=5.5 Hz, 1H), 3.43 (s, 3H), 3.35 (s, 6H), 2.94 (d, J=5.4 Hz, 2H), 2.92-2.89 (m, 1H), 2.87-2.77 (m, 1H), 2.77-2.65 (m, 1H), 2.26-2.17 (m, 1H).

Step 2. Synthesis of [1-(2,6-dioxopiperidin-3-yl)-3-methyl-2-oxo-2,3-dihydro-1H-benzimidazol-5-yl]acetaldehyde (P240): A mixture of C114 (1.29 g, 3.71 mmol), aqueous potassium hydrogen sulfate solution (1.0 M; 20 mL, 20 mmol), and ethyl acetate (25 mL) was stirred overnight at room temperature, whereupon LCMS analysis indicated the presence of C114: LCMS m/z 302.3 [M+H]⁺. The organic layer was washed with saturated aqueous sodium bicarbonate solution. The aqueous potassium hydrogen sulfate layer was diluted with dichloromethane (100 mL), then slowly treated with the aqueous sodium bicarbonate layer from above, under vigorously stirring. Saturated aqueous sodium bicarbonate solution was added to this mixture until the upper layer reached a pH of 7 to 8. The aqueous layer was further extracted with dichloromethane (2×50 mL), and all the dichloromethane and ethyl acetate layers were combined, dried over sodium sulfate, filtered, and concentrated in vacuo. Chromatography on silica gel (Gradient: 20% to 100% acetonitrile in dichloromethane) afforded P240 as a white, foam-like solid. Yield: 809 mg, 2.68 mmol, 72%. ¹H NMR (400 MHz, chloroform-d) δ 9.76 (s, 1H), 8.23 (br s, 1H), 6.87 (s, 1H), 6.85 (AB quartet, J_{AB}=7.9 Hz, Δv_{AB}=46.2 Hz, 2H), 5.22 (dd, J=12.6, 5.4 Hz, 1H), 3.74 (s, 2H), 3.43 (s, 3H), 3.01-2.64 (m, 3H), 2.29-2.18 (m, 1H).

### Preparation P241

### 3-[3-(2,4-Dioxo-1,3-diazinan-1-yl)pyrazolo[1,5-a]pyridin-6-yl]propanal (P241)

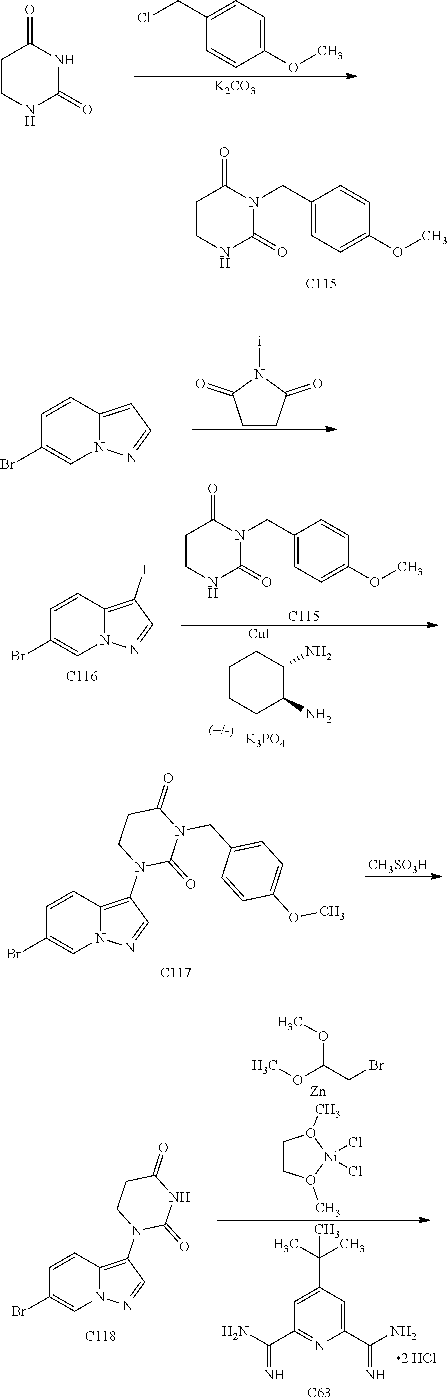

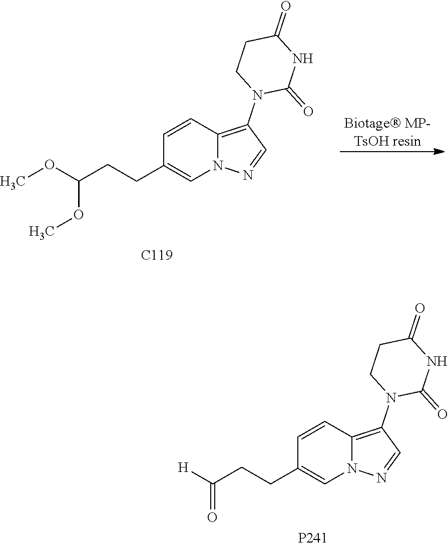 [SMILES: COC(CCc1ccc2c(N3CCC(=O)NC3=O)cnn2c1)OC ; "C119" ; "P241" ; O=CCCc1ccc2c(N3CCC(=O)NC3=O)cnn2c1]

Step 1. Synthesis of 3-[(4-methoxyphenyl)methyl]-1,3-diazinane-2,4-dione (C115): Potassium carbonate (12.1 g, 87.6 mmol) and 1-(chloromethyl)-4-methoxybenzene (9.61 g, 61.4 mmol) were added to a solution of 1,3-diazinane-2,4-dione (5.00 g, 43.8 mmol) in dimethyl sulfoxide (80 mL). After the reaction mixture had been stirred at 20° C. for 3 days, it was diluted with water (300 mL) and extracted with dichloromethane (3×300 mL). The combined organic layers were dried over sodium sulfate, filtered, concentrated in vacuo, and purified via silica gel chromatography (Gradient: 0% to 10% methanol in dichloromethane) to provide C115 as a white solid. Yield: 7.50 g, 32.0 mmol, 73%. LCMS m/z 235.1 [M+H]⁺. ¹H NMR (400 MHz, chloroform-d) δ 7.36 (d, J=8.7 Hz, 2H), 6.82 (d, J=8.7 Hz, 2H), 5.54 (br s, 1H), 4.88 (s, 2H), 3.78 (s, 3H), 3.37 (td, J=6.7, 1.6 Hz, 2H), 2.72 (t, J=6.8 Hz, 2H).

Step 2. Synthesis of 6-bromo-3-iodopyrazolo[1,5-a]pyridine (C116): To a solution of 6-bromopyrazolo[1,5-a]pyridine (6.00 g, 30.4 mmol) in acetonitrile (200 mL) was added N-iodosuccinimide (6.85 g, 30.4 mmol). The reaction mixture was stirred at 80° C. for 5 hours, whereupon it was concentrated in vacuo; the residue was purified via silica gel chromatography (Gradient: 0% to 10% ethyl acetate in petroleum ether) to afford C116 as a white solid. Yield: 9.80 g, 30.3 mmol, quantitative. LCMS m/z 322.9 (bromine isotope pattern observed) [M+H]⁺. ¹H NMR (400 MHz, DMSO-d₆) δ 9.16 (br s, 1H), 8.13 (s, 1H), 7.49 (d, half of AB quartet, J=9.4 Hz, 1H), 7.43 (dd, component of ABX system, J=9.4, 1.6 Hz, 1H).

Step 3. Synthesis of 1-(6-bromopyrazolo[1,5-a]pyridin-3-yl)-3-[(4-methoxyphenyl)methyl]-1,3-diazinane-2,4-dione (C117): To a solution of C116 (8.00 g, 24.8 mmol) in N,N-dimethylformamide (60 mL) were added C115 (8.12 g, 34.7 mmol), trans-cyclohexane-1,2-diamine (707 mg, 6.19 mmol), copper(I) iodide (1.18 g, 6.20 mmol), and tripotassium phosphate (13.1 g, 61.7 mmol), whereupon the reaction mixture was stirred at 80° C. for 16 hours. It was then concentrated in vacuo and purified via silica gel chromatography (Gradient: 0% to 50% ethyl acetate in petroleum ether), providing C117 as a yellow-brown solid. Yield: 7.40 g, 17.2 mmol, 69%. LCMS m/z 429.1 (bromine isotope pattern observed) [M+H]⁺. ¹H NMR (400 MHz, DMSO-d₆) δ 9.05 (dd, J=1.6, 0.9 Hz, 1H), 8.09 (s, 1H), 7.56 (dd, J=9.4, 0.9 Hz, 1H), 7.38 (dd, J=9.4, 1.6 Hz, 1H), 7.23 (d, J=8.6 Hz, 2H), 6.86 (d, J=8.7 Hz, 2H), 4.82 (s, 2H), 3.80 (t, J=6.7 Hz, 2H), 3.72 (s, 3H), 2.95 (t, J=6.7 Hz, 2H).

Step 4. Synthesis of 1-(6-bromopyrazolo[1,5-a]pyridin-3-yl)-1,3-diazinane-2,4-dione (C118): A 25° C. solution of C117 (7.40 g, 17.2 mmol) (80 mL) in toluene was treated with methanesulfonic acid (20 mL) and then stirred at 110° C. for 2 hours. After removal of the upper (toluene) layer, the remaining layer was cooled in an ice bath and treated with methanol (80 mL).

The precipitate was collected via filtration to afford C118 as a white solid. Yield: 4.30 g, 13.9 mmol, 81%. LCMS m/z 308.9 (bromine isotope pattern observed) [M+H]⁺. ¹H NMR (400 MHz, DMSO-d₆) δ 10.47 (s, 1H), 9.05 (dd, J=1.7, 0.9 Hz, 1H), 8.07 (s, 1H), 7.60 (dd, J=9.4, 0.9 Hz, 1H), 7.37 (dd, J=9.4, 1.6 Hz, 1H), 3.78 (t, J=6.7 Hz, 2H), 2.77 (t, J=6.7 Hz, 2H).

Step 5. Synthesis of 1-[6-(3,3-dimethoxypropyl)pyrazolo[1,5-a]pyridin-3-yl]-1,3-diazinane-2,4-dione (C119): To a vial containing C118 (450 mg, 1.46 mmol), nickel(II) chloride, ethylene glycol dimethyl ether complex (32.0 mg, 0.146 mmol), zinc flakes (192 mg, 2.94 mmol), C63 (43.5 mg, 0.149 mmol), and 3-bromo-1,1-dimethoxypropane (0.397 mL, 2.91 mmol) was added N,N-dimethylacetamide (1.6 mL), whereupon the suspension was degassed by bubbling nitrogen through it for 1 minute. After the reaction mixture had been heated at 65° C. overnight, it was filtered through diatomaceous earth and the filter cake was washed with ethyl acetate (20 mL). The combined filtrates were subjected to a second filtration through diatomaceous earth, and this filtrate was concentrated in vacuo to a syrup, which was dissolved in minimal dichloromethane and subjected to chromatography on silica gel (Gradient: 0% to 100% acetonitrile in dichloromethane), affording C119 as a white foam (431 mg). This material contained impurities, and was taken directly to the following step. LCMS m/z 333.3 [M+H]⁺. ¹H NMR (400 MHz, chloroform-d), product peaks only: δ 8.33 (br s, 1H), 7.92 (s, 1H), 7.57 (br s, 1H), 7.38 (d, J=9.1 Hz, 1H), 7.10 (br d, J=9.1 Hz, 1H), 4.40 (t, J=5.6 Hz, 1H), 3.89 (t, J=6.7 Hz, 2H), 3.35 (s, 6H), 2.89 (t, J=6.7 Hz, 2H), 2.73-2.66 (m, 2H), 1.98-1.90 (m, 2H).

Step 6. Synthesis of 3-[3-(2,4-dioxo-1,3-diazinan-1-yl)pyrazolo[1,5-a]pyridin-6-yl]propanal (P241): To a mixture of C119 (from the previous step; 431 mg, 51.3 mmol) in a mixture of tetrahydrofuran (5 mL) and water (0.5 mL) was added Biotage® MP-TsOH resin (microporous polystyrene resin containing bound para-toluenesulfonic acid; 1.00 g, 1.46 mmol/gram load factor). After the reaction mixture had been stirred at room temperature for 21 hours, it was diluted with dichloromethane (20 mL), dried over magnesium sulfate, filtered, and concentrated in vacuo, providing P241 as a tan solid (320 mg). The purity of this material was estimated at approximately 80%; it was used in further chemistry without additional purification. LCMS m/z 287.3 [M+H]⁺. ¹H NMR (400 MHz, DMSO-d₆), product peaks only, characteristic peaks: δ 10.42 (s, 1H), 9.73 (s, 1H), 8.50 (s, 1H), 7.96 (s, 1H), 7.53 (d, J=9.1 Hz, 1H), 7.17 (br d, J=9.1 Hz, 1H), 3.76 (t, J=6.7 Hz, 2H), 2.78-2.74 (m, 2H).

### Preparation P242

### 3-[3-(2,4-Dioxo-1,3-diazinan-1-yl)imidazo[1,2-a]pyridin-7-yl]propanal (P242)

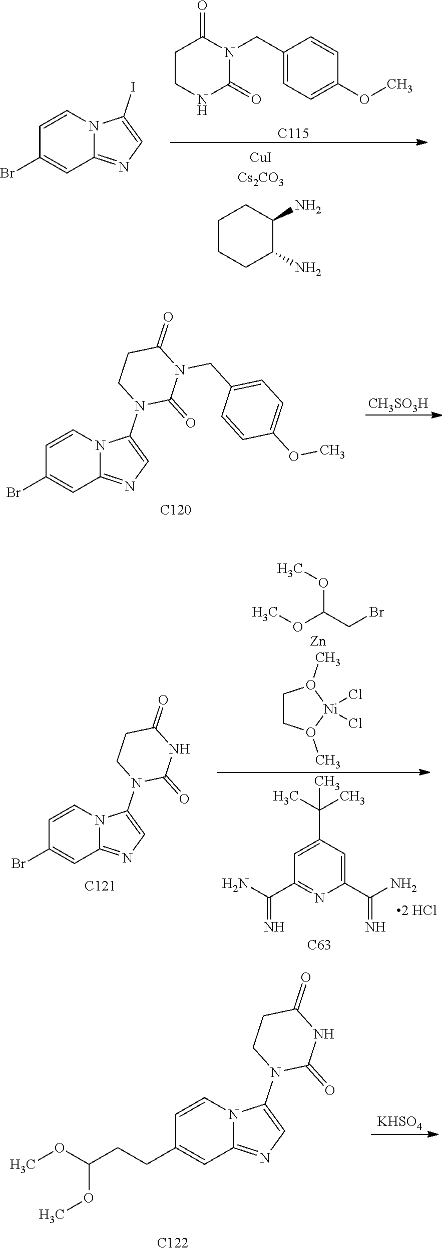

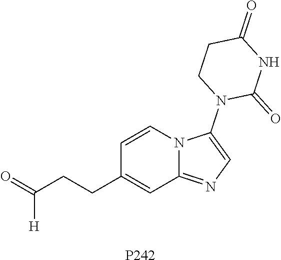 [SMILES: "P242" ; O=CCCc1ccn2c(N3CCC(=O)NC3=O)cnc2c1]

Step 1. Synthesis of 1-(7-bromoimidazo[1,2-a]pyridin-3-yl)-3-[(4-methoxyphenyl)methyl]-1,3-diazinane-2,4-dione (C120): To a solution of 7-bromo-3-iodoimidazo[1,2-a]pyridine (60.0 g, 186 mmol) and C115 (47.9 g, 204 mmol) in toluene (800 mL) were added (1R,2R)-cyclohexane-1,2-diamine (5.30 g, 46.4 mmol), copper(I) iodide (5.31 g, 27.9 mmol), and cesium carbonate (151 g, 463 mmol). After the reaction mixture had been stirred at 100° C. for 40 hours, it was partitioned between water (1 L) and ethyl acetate (1 L) and filtered. The aqueous layer was extracted with ethyl acetate (3×1 L), and the combined organic layers were washed with saturated aqueous sodium chloride solution (1 L), dried over magnesium sulfate, filtered, and concentrated in vacuo. Silica gel chromatography (Gradient: 35% to 55% tetrahydrofuran in petroleum ether) afforded C120 as a yellow oil. Yield: 40 g, 93 mmol, 50%.

Step 2. Synthesis of 1-(7-bromoimidazo[1,2-a]pyridin-3-yl)-1,3-diazinane-2,4-dione (C121): To a solution of C120 (40 g, 93 mmol) in toluene (260 mL) was added methanesulfonic acid (125 mL). The reaction mixture was stirred at 110° C. for 2 hours, whereupon it was concentrated in vacuo to remove toluene; the residue was added drop-wise into stirring aqueous sodium carbonate solution (20%, 800 mL). After the resulting mixture had been filtered, the filter cake was washed with water (5×100 mL), then azeotroped under reduced pressure with toluene (5×100 mL). The residue was stirred with methyl tert-butyl ether (500 mL) at 25° C. for 16 hours, then collected by filtration, to provide C121 as a gray solid. Yield: 25.5 g, 82.5 mmol, 89%. LCMS m/z 309.0 (bromine isotope pattern observed) [M+H]⁺. ¹H NMR (400 MHz, DMSO-d₆) δ 10.68 (s, 1H), 8.31 (br d, J=7.3 Hz, 1H), 7.92 (br d, J=2.0 Hz, 1H), 7.58 (s, 1H), 7.14 (dd, J=7.2, 2.0 Hz, 1H), 3.80 (t, J=6.7 Hz, 2H), 2.82 (t, J=6.7 Hz, 2H).

Step 3. Synthesis of 1-[7-(3,3-dimethoxypropyl)imidazo[1,2-a]pyridin-3-yl]-1,3-diazinane-2,4-dione (C122): A mixture of C121 (300 mg, 0.970 mmol), nickel(II) chloride, ethylene glycol dimethyl ether complex (213 mg, 0.969 mmol), C63 (284 mg, 0.972 mmol), and zinc (635 mg, 9.71 mmol) was degassed with nitrogen, whereupon a solution of 3-bromo-1,1-dimethoxypropane (444 mg, 2.43 mmol) in N,N-dimethylacetamide (20 mL) was added via syringe. After being degassed with nitrogen again, the reaction mixture was heated to 65° C. for 14 hours, then diluted with ethyl acetate (30 mL) and filtered through diatomaceous earth. The filter cake was washed with ethyl acetate, and the combined filtrates were washed with saturated aqueous sodium chloride solution (20 mL), dried over sodium sulfate, filtered, and concentrated in vacuo. Chromatography on silica gel (Gradient: 0% to 8% methanol in dichloromethane) afforded C122 as a white solid. Yield: 120 mg, 0.361 mmol, 37%. LCMS m/z 333.2 [M+H]⁺.

Step 4. Synthesis of 3-[3-(2,4-dioxo-1,3-diazinan-1-yl)imidazo[1,2-a]pyridin-7-yl]propanal (P242): Aqueous potassium hydrogen sulfate solution (1 M; 2.5 mL, 2.5 mmol) was added to a solution of C122 (120 mg, 0.361 mmol) in ethyl acetate (3 mL), and the reaction mixture was stirred at 25° C. for 4 hours. Water (10 mL) was then added, and the resulting mixture was extracted with ethyl acetate (2×10 mL); the combined organic layers were washed with saturated aqueous sodium chloride solution, dried over sodium sulfate, filtered, and concentrated in vacuo to provide P242 as a solid. Yield: 50.0 mg, 0.175 mmol, 48%. LCMS m/z 287.1 [M+H]⁺. ¹H NMR (400 MHz, DMSO-d₆) δ 10.59 (br s, 1H), 9.74 (s, 1H), 8.21 (d, J=7.0 Hz, 1H), 7.48 (s, 1H), 7.39 (br s, 1H), 6.88 (br d, J=7.0 Hz, 1H), 3.77 (t, J=6.7 Hz, 2H), 2.97-2.76 (m, 6H).

### Preparation P243

### 1-{2-Methyl-5-[3-(piperazin-1-yl)-1-oxa-8-azaspiro[4.5]decane-8-carbonyl]phenyl}-1,3-diazinane-2,4-dione, hydrochloride salt (P243)

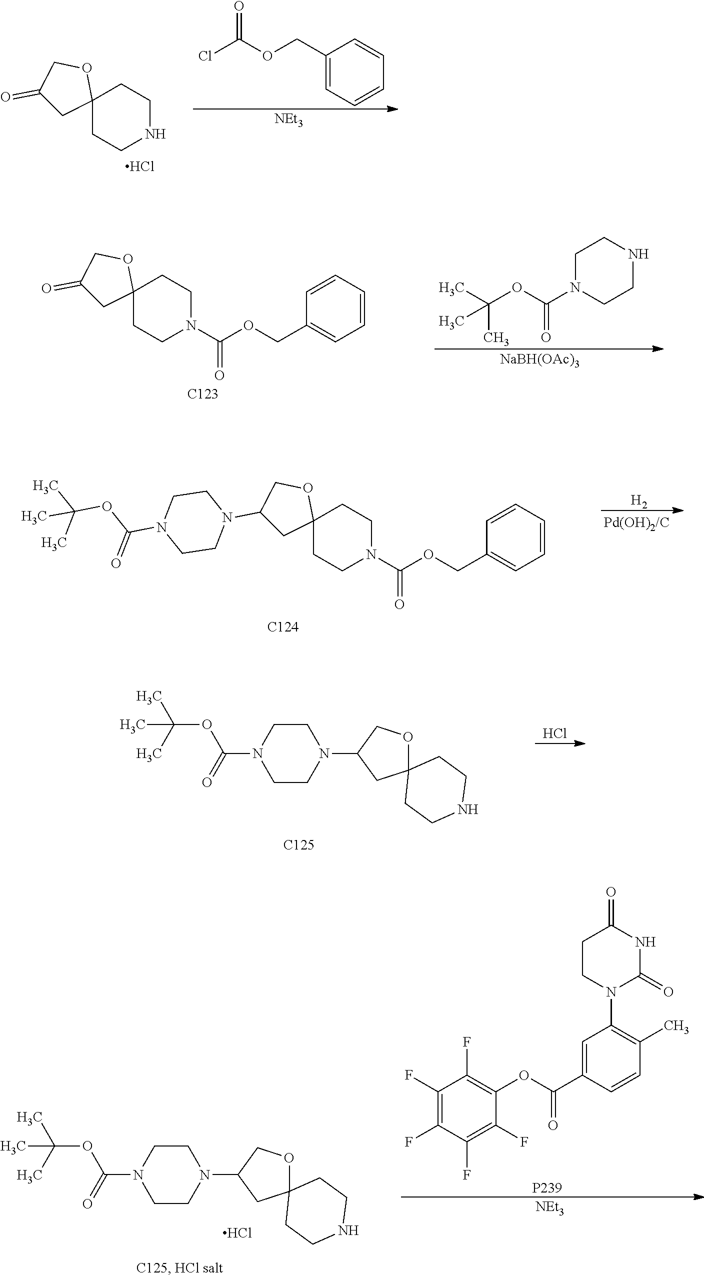

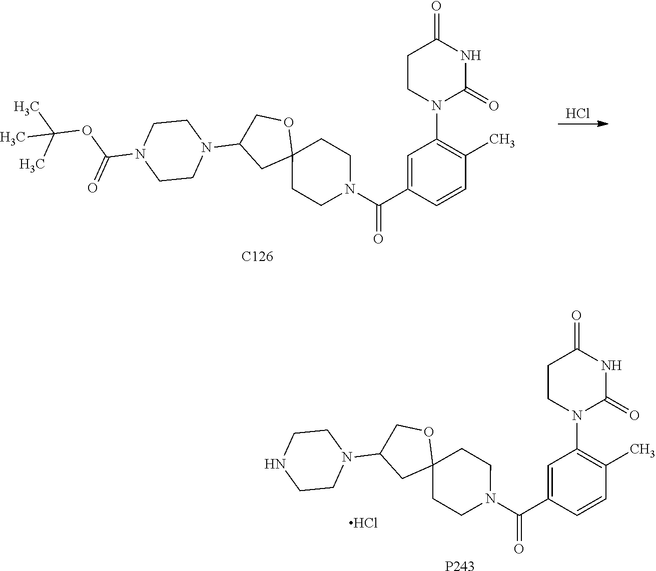 [SMILES: Cc1ccc(C(=O)N2CCC3(CC2)CC(N2CCN(C(=O)OC(C)(C)C)CC2)CO3)cc1N1CCC(=O)NC1=O ; Cl ; "C126" ; "P243" ; Cc1ccc(C(=O)N2CCC3(CC2)CC(N2CCNCC2)CO3)cc1N1CCC(=O)NC1=O ; Cl]

Step 1. Synthesis of benzyl 3-oxo-1-oxa-8-azaspiro[4.5]decane-8-carboxylate (C123): Benzyl chloroformate (95%, 0.862 mL, 5.74 mmol) was added to a solution of 1-oxa-8-azaspiro[4.5]decan-3-one, hydrochloride salt (500 mg, 2.61 mmol) and triethylamine (364 μL, 2.61 mmol) in dichloromethane (5.0 mL). After the reaction mixture had been stirred at room temperature overnight, it was partitioned between dichloromethane (20 mL) and saturated aqueous sodium bicarbonate solution (40 mL), and the aqueous layer was extracted with dichloromethane (3×10 mL). The combined organic layers were washed with saturated aqueous sodium chloride solution (10 mL), dried over sodium sulfate, filtered, and concentrated in vacuo. Silica gel chromatography (Gradient: 0% to 75% ethyl acetate in heptane), followed by reconcentration of the purified product from dichloromethane (20 mL), afforded C123 as a thick, colorless oil. Yield: 348 mg, 1.20 mmol, 46%. GCMS m/z 289.2 [M+]. ¹H NMR (400 MHz, DMSO-d₆) δ 7.41-7.28 (m, 5H), 5.08 (s, 2H), 3.99 (s, 2H), 3.59-3.49 (m, 2H), 3.46-3.32 (m, 2H), 2.44 (s, 2H), 1.75-1.61 (m, 4H).

Step 2. Synthesis of benzyl 3-[4-(tert-butoxycarbonyl)piperazin-1-yl]-1-oxa-8-azaspiro[4.5]decane-8-carboxylate (C124): Sodium triacetoxyborohydride (693 mg, 3.27 mmol) was added to a solution of C123 (338 mg, 1.17 mmol) and tert-butyl piperazine-1-carboxylate (218 mg, 1.17 mmol) in 1,2-dichloroethane (5.0 mL), and the reaction mixture was stirred at room temperature for 16 hours, whereupon it was partitioned between saturated aqueous sodium bicarbonate solution (25 mL) and dichloromethane (20 mL). The aqueous layer was extracted with dichloromethane (2×10 mL), and the combined organic layers were washed with saturated aqueous sodium chloride solution (10 mL), dried over sodium sulfate, filtered, and concentrated in vacuo. Silica gel chromatography (Gradient: 0% to 100% ethyl acetate in heptane) was followed by reconcentration of the purified product from dichloromethane (20 mL), providing C124 as a thick, colorless oil. Yield: 457 mg, 0.994 mmol, 85%. LCMS m/z 460.4 [M+H]⁺. ¹H NMR (400 MHz, DMSO-d₆) δ 7.41-7.28 (m, 5H), 5.06 (s, 2H), 3.90 (dd, J=8.5, 6.7 Hz, 1H), 3.59-3.47 (m, 3H), 3.31-3.24 (m, 6H), 2.91 (p, J=7.7 Hz, 1H), 2.37-2.28 (m, 2H), 2.27-2.19 (m, 2H), 2.00-1.92 (m, 1H), 1.61-1.42 (m, 5H), 1.38 (s, 9H).

Step 3. Synthesis of tert-butyl 4-(1-oxa-8-azaspiro[4.5]decan-3-yl)piperazine-1-carboxylate (C125): Palladium hydroxide on carbon (20%, 68.1 mg, 97.0 μmol) was added to a solution of C124 (446 mg, 0.970 mmol) in methanol (10 mL). After the mixture had been purged three times with nitrogen, then purged three times with hydrogen, it was hydrogenated in a Parr reactor at 30 psi for 16 hours at room temperature. The reaction mixture was filtered through diatomaceous earth, and the filter pad was rinsed with methanol; the combined filtrates were concentrated under reduced pressure. The resulting oil was treated with ethyl acetate to provide a solid; the mixture was concentrated in vacuo, dissolved in dichloromethane, and passed through a 0.2 μm membrane filter. The filtrate was concentrated in vacuo to afford C125 as a white solid. Yield: 320 mg, assumed quantitative. LCMS m/z 326.3 [M+H]⁺. ¹H NMR (400 MHz, DMSO-d₆) δ 3.87 (dd, J=8.5, 6.7 Hz, 1H), 3.51 (t, J=8.1 Hz, 1H), 3.31-3.24 (m, 4H), 2.94-2.75 (m, 3H), 2.71-2.60 (m, 2H), 2.37-2.28 (m, 2H), 2.27-2.18 (m, 2H), 1.95 (dd, J=12.3, 7.8 Hz, 1H), 1.63-1.42 (m, 5H), 1.39 (s, 9H).

Step 4. Synthesis of tert-butyl 4-(1-oxa-8-azaspiro[4.5]decan-3-yl)piperazine-1-carboxylate, hydrochloride salt (C125, HCl salt): A −78° C. solution of C125 (100 mg, 0.307 mmol) in dichloromethane (2.0 mL) was treated with a solution of hydrogen chloride in 1,4-dioxane (4.0 M; 77 μL, 0.31 mmol). After the reaction mixture had been stirred at −78° C. for 2 minutes, it was concentrated in vacuo below room temperature, providing C125, HCl salt as a hard, white solid. Yield: 106 mg, 0.293 mmol, 95%. LCMS m/z 326.4 [M+H]⁺. ¹H NMR (400 MHz, DMSO-d₆) δ 8.98 (br s, 2H), 3.91 (dd, J=8.6, 6.6 Hz, 1H), 3.60-3.52 (m, 1H), 3.33-3.24 (m, 4H), 3.09-2.89 (m, 5H), 2.39-2.29 (m, 2H), 2.29-2.20 (m, 2H), 2.00 (dd, J=12.5, 7.8 Hz, 1H), 1.90-1.68 (m, 4H), 1.59 (dd, J=12.5, 8.3 Hz, 1H), 1.38 (s, 9H).

Step 5. Synthesis of tert-butyl 4-{8-[3-(2,4-dioxo-1,3-diazinan-1-yl)-4-methylbenzoyl]-1-oxa-8-azaspiro[4.5]decan-3-yl}piperazine-1-carboxylate (C126): To a solution of C125, HCl salt (510 mg, 1.41 mmol) in N,N-dimethylformamide (6.0 mL) was added triethylamine (1.09 mL, 7.82 mmol); after the resulting mixture had been stirred for 5 minutes, P239 (649 mg, 1.57 mmol) was added. The reaction mixture was stirred at room temperature for 4.5 hours, whereupon it was added to a mixture of water (50 mL) and saturated aqueous sodium chloride solution (10 mL). After the suspension had been stirred for 30 minutes, it was extracted with ethyl acetate (3×50 mL), and the combined organic layers were dried over sodium sulfate. The solvent was decanted off and concentrated under reduced pressure to provide C126 as a solid (1.10 g). Most of this material was used in the following step. LCMS m/z 556.4 [M+H]⁺. ¹H NMR (400 MHz, DMSO-d₆), characteristic peaks: δ 10.37 (s, 1H), 7.36-7.30 (m, 2H), 7.25 (dd, J=7.7, 1.8 Hz, 1H), 2.21 (s, 3H), 2.03-1.94 (m, 1H), 1.38 (s, 9H).

Step 6. Synthesis of 1-{2-methyl-5-[3-(piperazin-1-yl)-1-oxa-8-azaspiro[4.5]decane-8-carbonyl]phenyl}-1,3-diazinane-2,4-dione, hydrochloride salt (P243): A solution of hydrogen chloride in 1,4-dioxane (4 M; 12 mL, 48 mmol) was slowly added to a 0° C. solution of C126 (from the previous step; 875.0 mg, 51.12 mmol) in dichloromethane (10 mL). After the reaction mixture had warmed to room temperature and been stirred for 2 hours, it was concentrated in vacuo, providing P243 as a solid. Yield: 453 mg, 0.921 mmol, 82% over 2 steps. LCMS m/z 456.3 [M+H]⁺. ¹H NMR (400 MHz, DMSO-d₆), characteristic peaks: δ 10.37 (s, 1H), 9.66-9.22 (br m, 2H), 7.37-7.32 (m, 2H), 7.27 (dd, J=7.7, 1.8 Hz, 1H), 2.22 (s, 3H), 2.09-1.89 (m, 1H).

### Preparation P244

### 1-(2,6-Dioxopiperidin-3-yl)-3-methyl-2-oxo-2,3-dihydro-1H-benzimidazole-4-carbaldehyde (P244)

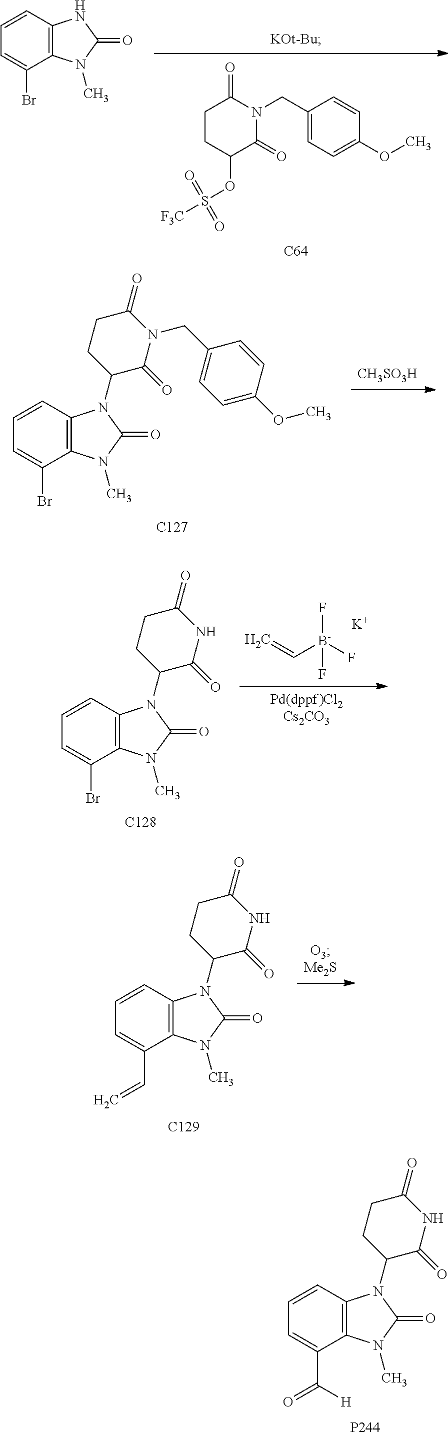

Step 1. Synthesis of 3-(4-bromo-3-methyl-2-oxo-2,3-dihydro-1H-benzimidazol-1-yl)-1-[(4-methoxyphenyl)methyl]piperidine-2,6-dione (C127): Potassium tert-butoxide (35.3 g, 315 mmol) was added portion-wise to a 0° C. to 5° C. solution of 7-bromo-1-methyl-1,3-dihydro-2H-benzimidazol-2-one (65.0 g, 286 mmol) in tetrahydrofuran (650 mL). The reaction mixture was stirred at 0° C. to 5° C. for 1 hour, whereupon a solution of C64 (120 g, 315 mmol) in tetrahydrofuran (1 L) was added drop-wise while the reaction mixture was maintained at 0° C. to 5° C. It was then warmed to 25° C. and stirred for 16 hours, before being cooled to 0° C. to 5° C. Water (1.5 L) was added in portions, followed by ethyl acetate (1.2 L), and the aqueous layer was extracted with ethyl acetate (3×1 L). The combined organic layers were washed with saturated aqueous sodium chloride solution (1 L), dried over magnesium sulfate, filtered, concentrated in vacuo, and purified using silica gel chromatography (Eluent: 3:1 petroleum ether/tetrahydrofuran) to afford C127 as an off-white solid. Yield: 50.0 g, 109 mmol, 38%. ¹H NMR (400 MHz, DMSO-d₆) δ 7.24 (br d, J=8.1 Hz, 1H), 7.20 (d, J=8.7 Hz, 2H), 7.08 (br d, J=7.9 Hz, 1H), 6.94 (t, J=8.0 Hz, 1H), 6.85 (d, J=8.7 Hz, 2H), 5.57 (dd, J=13.0, 5.3 Hz, 1H), 4.79 (AB quartet, J_{AB}=14.4 Hz, Δv_{AB}=25.0 Hz, 2H), 3.72 (s, 3H), 3.63 (s, 3H), 3.11-2.98 (m, 1H), 2.88-2.65 (m, 2H), 2.12-2.02 (m, 1H).

Step 2. Synthesis of 3-(4-bromo-3-methyl-2-oxo-2,3-dihydro-1H-benzimidazol-1-yl)piperidine-2,6-dione (C128): To a solution of C127 (50.0 g, 109 mmol) in toluene (500 mL) was added methanesulfonic acid (311 mL, 4.79 mol) drop-wise, whereupon the reaction mixture was heated at 100° C. for 3 hours. After cooling to room temperature, it was poured into water (900 mL), and the aqueous layer was extracted with ethyl acetate (3×600 mL); the combined organic layers were washed with saturated aqueous sodium chloride solution (600 mL), dried over magnesium sulfate, filtered, and concentrated in vacuo. Silica gel chromatography (Gradient: 0% to 100% ethyl acetate in petroleum ether) provided C128 as an off-white solid. Yield: 25 g, 74 mmol, 68%. LCMS m/z 340.2 (bromine isotope pattern observed) [M+H]⁺. ¹H NMR (400 MHz, DMSO-d₆) δ 11.14 (s, 1H), 7.24 (d, J=8.1 Hz, 1H), 7.17 (d, J=7.9 Hz, 1H), 6.98 (t, J=8.0 Hz, 1H), 5.41 (dd, J=12.6, 5.3 Hz, 1H), 3.63 (s, 3H), 2.95-2.82 (m, 1H), 2.78-2.58 (m, 2H), 2.08-1.99 (m, 1H).

Step 3. Synthesis of 3-(4-ethenyl-3-methyl-2-oxo-2,3-dihydro-1H-benzimidazol-1-yl)piperidine-2,6-dione (C129): To a solution of C128 (40.0 g, 118 mmol) and potassium (ethenyl)trifluoroborate (23.8 g, 178 mmol) in a mixture of 1,4-dioxane (3.2 L) and water (32 mL) were added [1,1′-bis(diphenylphosphino)ferrocene]dichloropalladium(II), dichloromethane complex (9.59 g, 11.7 mmol) and cesium carbonate (77.1 g, 237 mmol). The reaction mixture was stirred at 90° C. for 6 hours, whereupon it was filtered; the filtrate was concentrated in vacuo and subjected to chromatography on silica gel (Gradient: 0% to 50% ethyl acetate in dichloromethane), affording C129 as a yellow solid. Yield: 19.9 g, 69.8 mmol, 59%. LCMS m/z 286.1 [M+H]⁺. ¹H NMR (400 MHz, DMSO-d₆) δ 11.11 (s, 1H), 7.40 (dd, J=17.2, 10.9 Hz, 1H), 7.19 (br d, J=7.4 Hz, 1H), 7.10-7.00 (m, 2H), 5.72 (d, J=17.2 Hz, 1H), 5.44-5.34 (m, 2H), 3.54 (s, 3H), 2.89 (ddd, J=17.3, 13.1, 5.3 Hz, 1H), 2.78-2.58 (m, 2H), 2.07-1.96 (m, 1H).

Step 4. Synthesis of 1-(2,6-dioxopiperidin-3-yl)-3-methyl-2-oxo-2,3-dihydro-1H-benzimidazole-4-carbaldehyde (P244): A solution of C129 (19.9 g, 69.8 mmol) in dichloromethane (3.2 L) was stirred under ozone at −78° C. for 30 minutes, whereupon dimethyl sulfide (100 mL) was added at −78° C. After the reaction mixture had been stirred at 25° C. for 16 hours, it was combined with a similar reaction carried out using C129 (6.80 g, 23.8 mmol) and filtered. The filter cake was washed with dichloromethane (200 mL) and water (100 mL), and the combined organic filtrates were washed with saturated aqueous sodium chloride solution (2×150 mL), dried over sodium sulfate, filtered, and concentrated in vacuo. The residue was triturated with dichloromethane (50 mL), providing P244 as a light-yellow solid. Combined yield: 23.9 g, 83.2 mmol, 89%. LCMS m/z 288.1 [M+H]⁺. ¹H NMR (400 MHz, DMSO-d₆) δ 11.17 (s, 1H), 10.40 (s, 1H), 7.60 (d, J=8.1 Hz, 1H), 7.46 (d, J=7.8 Hz, 1H), 7.21 (t, J=7.9 Hz, 1H), 5.47 (dd, J=12.7, 5.4 Hz, 1H), 3.67 (s, 3H), 2.90 (ddd, J=17.0, 13.3, 5.2 Hz, 1H), 2.82-2.59 (m, 2H), 2.11-2.00 (m, 1H).

### Preparation P245

### 1-(2,6-Dioxopiperidin-3-yl)-3-methyl-2-oxo-2,3-dihydro-1H-benzimidazole-5-carbaldehyde (P245)

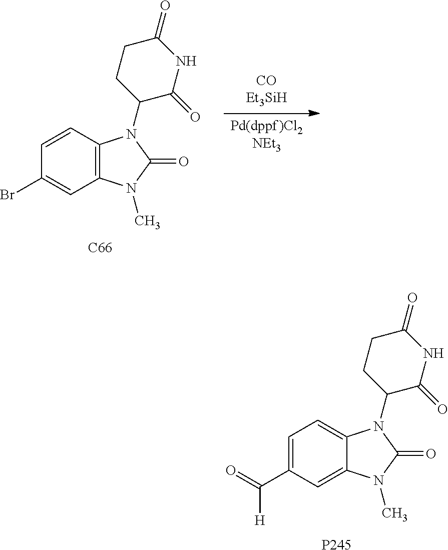 [SMILES: "C66" ; "P245" ; Cn1c(=O)n(C2CCC(=O)NC2=O)c2ccc(Br)cc21 ; Cn1c(=O)n(C2CCC(=O)NC2=O)c2ccc(C=O)cc21 ; "Pd(dppf)Cl2NEt3" ; "COEt3SiH"]

Triethylamine (80.3 mL, 576 mmol), [1,1′-bis(diphenylphosphino)ferrocene]dichloropalladium(II) (14.1 g, 19.3 mmol), and triethylsilane (92.1 mL, 577 mmol) were added to a solution of C66 (65.0 g, 192 mmol) in N,N-dimethylformamide (650 mL). The reaction mixture was degassed under vacuum and then purged with argon; this evacuation-purge cycle was carried out a total of three times, whereupon the reaction mixture was stirred under carbon monoxide (50 psi) at 80° C. for 24 hours. After cooling to room temperature, it was poured into a mixture of 1 M hydrochloric acid (600 mL) and water (1.2 L); filtration was followed by washing of the filter cake with water (3×100 mL). The remaining solid was dried at 50° C. to remove residual water, stirred with ethanol (200 mL) at 50° C. for 16 hours, and filtered. The resulting filter cake was stirred with acetonitrile (300 mL) at 25° C. for 30 minutes and filtered, providing P245 as a brown solid. Yield: 30.2 g, 105 mmol, 55%. LCMS m/z 288.3 [M+H]⁺. ¹H NMR (400 MHz, DMSO-d₆) δ 11.18 (br s, 1H), 9.94 (s, 1H), 7.73-7.66 (m, 2H), 7.36 (d, J=8.4 Hz, 1H), 5.48 (dd, J=12.8, 5.4 Hz, 1H), 3.42 (s, 3H), 2.97-2.84 (m, 1H), 2.81-2.59 (m, 2H), 2.12-2.02 (m, 1H).

### Preparation P246

### 1-[6-(3-Hydroxypropyl)pyrazolo[1,5-a]pyridin-3-yl]-1,3-diazinane-2,4-dione (P246)

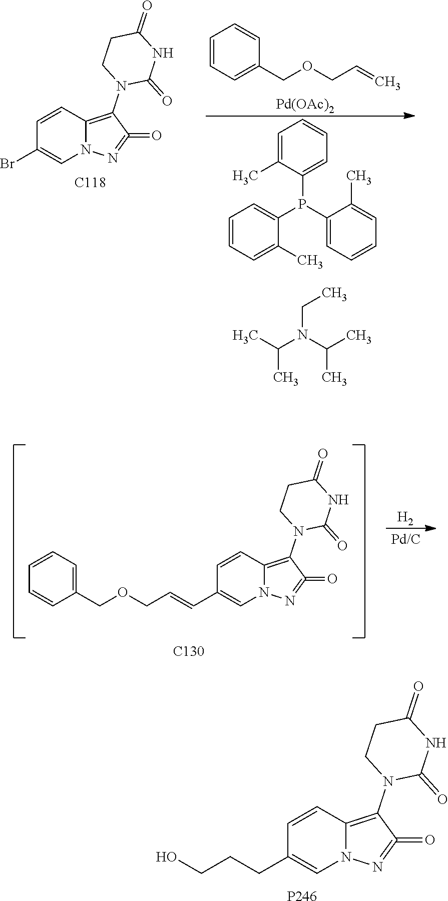

A mixture of C118 (1.25 g, 4.04 mmol), {[(prop-2-en-1-yl)oxy]methyl}benzene (719 mg, 4.85 mmol), palladium(II) acetate (90.8 mg, 0.404 mmol), tri-o-tolylphosphine (246 mg, 0.808 mmol), and N,N-diisopropylethylamine (2.61 g, 20.2 mmol) in N,N-dimethylformamide (10 mL) was stirred at 100° C. for 16 hours. After the reaction mixture had been diluted with water (100 mL) and extracted with ethyl acetate (2×50 mL), the combined organic layers were washed with saturated aqueous sodium chloride solution (50 mL), dried over sodium sulfate, filtered, and concentrated in vacuo. Silica gel chromatography (Gradient: 0% to 100% ethyl acetate in petroleum ether) provided C130 {1-{6-[(1E)-3-(benzyloxy)prop-1-en-1-yl]pyrazolo[1,5-a]pyridin-3-yl}-1,3-diazinane-2,4-dione} as a yellow solid (1.25 g). LCMS m/z 377.2 [M+H]⁺.

A portion of this material was taken on. A mixture of C130 (500 mg) and palladium on carbon (100 mg) in methanol (30 mL) was stirred at 25° C. for 7 hours, then at 60° C. for 3 hours. The reaction mixture was filtered, and the filtrate was concentrated in vacuo; purification via silica gel chromatography (Gradient: 0% to 20% methanol in dichloromethane) afforded P246 as a white solid. Yield: 200 mg, 0.694 mmol, 43%. LCMS m/z 289.1 [M+H]⁺. ¹H NMR (400 MHz, DMSO-d₆) δ 10.43 (s, 1H), 8.45 (s, 1H), 7.95 (s, 1H), 7.52 (br d, J=9.1 Hz, 1H), 7.16 (dd, J=9.1, 1.4 Hz, 1H), 4.53 (t, J=5.1 Hz, 1H), 3.76 (t, J=6.7 Hz, 2H), 3.47-3.40 (m, 2H), 2.76 (t, J=6.7 Hz, 2H), 2.64 (t, J=7.6 Hz, 2H), 1.80-1.70 (m, 2H).

### Preparation P247

### tert-Butyl (3R)-3-(piperazin-1-yl)-1-oxa-8-azaspiro[4.5]decane-8-carboxylate (P247)

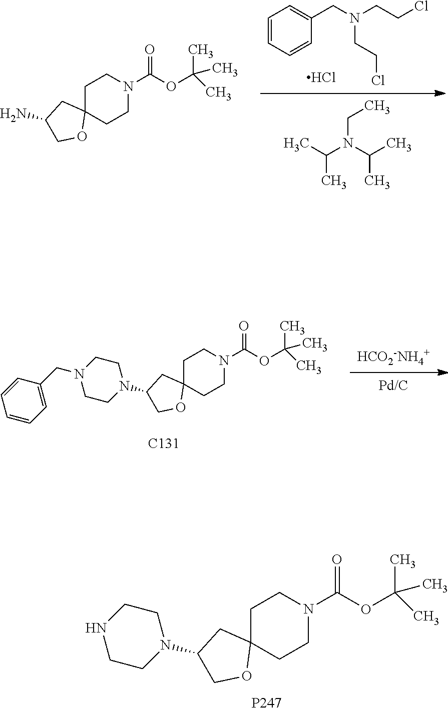

Step 1. Synthesis of tert-butyl (3R)-3-(4-benzylpiperazin-1-yl)-1-oxa-8-azaspiro[4.5]decane-8-carboxylate (C131): A solution of N-benzyl-2-chloro-N-(2-chloroethyl)ethan-1-amine, hydrochloride salt (5.76 g, 21.4 mmol), tert-butyl (3R)-3-amino-1-oxa-8-azaspiro[4.5]decane-8-carboxylate (see J. T. Kohrt et al., Org. Process Res. Dev. 2022, 26, 616-623; 5.00 g, 19.5 mmol), and N,N-diisopropylethylamine (13.8 mL, 79.2 mmol) in N,N-dimethylformamide (5 mL) was heated at 120° C. for 6 hours, cooled, and stirred at room temperature for 3 days. After the reaction mixture had been diluted with ethyl acetate (500 mL), it was washed sequentially with aqueous sodium hydroxide solution (0.5 M; 300 mL) and saturated aqueous sodium chloride solution, dried over sodium sulfate, filtered, and concentrated in vacuo. Silica gel chromatography (Gradient: 0% to 20% methanol in dichloromethane) afforded C131 as a tan oil. Yield: 5.0 g, 12 mmol, 62%. LCMS m/z 416.4 [M+H]⁺. ¹H NMR (400 MHz, chloroform-d), characteristic peaks: δ 7.34-7.28 (m, 4H), 7.28-7.21 (m, 1H), 3.99 (dd, J=8.6, 6.7 Hz, 1H), 3.66 (t, J=8.4 Hz, 1H), 3.51 (s, 2H), 3.37-3.21 (m, 2H), 3.02-2.89 (m, 1H), 1.95 (dd, J=12.2, 7.7 Hz, 1H), 1.71-1.55 (m, 4H), 1.52-1.44 (m, 1H), 1.44 (s, 9H).

Step 2. Synthesis of tert-butyl (3R)-3-(piperazin-1-yl)-1-oxa-8-azaspiro[4.5]decane-8-carboxylate (P247): A mixture of C131 (2.5 g, 6.0 mmol), ammonium formate (98%, 3.80 g, 59.1 mmol), and palladium on carbon (500 mg) in methanol (50 mL) was stirred for 2 days at 50° C., whereupon the reaction mixture was filtered through diatomaceous earth and concentrated in vacuo. After the residue had been diluted with diethyl ether and heptane, it was concentrated again, providing P247 as an off-white solid. Yield: 1.89 g, 5.81 mmol, 97%. LCMS m/z 326.4 [M+H]⁺. ¹H NMR (400 MHz, DMSO-d₆) δ 3.89 (dd, J=8.5, 6.6 Hz, 1H), 3.52 (t, J=8.1 Hz, 1H), 3.47-3.37 (m, 2H), 3.29-3.11 (m, 2H), 2.97-2.86 (m, 1H), 2.83 (t, J=5.0 Hz, 4H), 2.48-2.38 (m, 2H), 2.38-2.29 (m, 2H), 1.96 (dd, J=12.3, 7.8 Hz, 1H), 1.57-1.45 (m, 4H), 1.45-1.39 (m, 1H), 1.38 (s, 9H).

### Preparation P248

### 3-(2,4-Dioxo-1,3-diazinan-1-yl)pyrazolo[1,5-a]pyridine-6-carbaldehyde (P248)

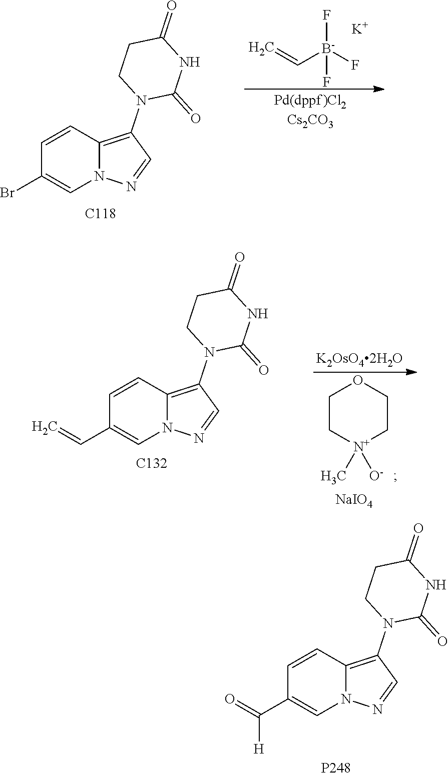

Step 1. Synthesis of 1-(6-ethenylpyrazolo[1,5-a]pyridin-3-yl)-1,3-diazinane-2,4-dione (C132): To a solution of C118 (2.0 g, 6.47 mmol) in a mixture of 1,4-dioxane (50 mL) and water (10 mL) was added cesium carbonate (4.22 g, 13.0 mmol), [1,1′-bis(diphenylphosphino)ferrocene]dichloropalladium(II), dichloromethane complex (524 mg, 0.642 mmol), and potassium (ethenyl)trifluoroborate (2.17 g, 16.2 mmol). The reaction mixture was stirred at 80° C. for 6 hours, whereupon it was concentrated in vacuo and extracted with ethyl acetate (3×100 mL). The combined organic layers were dried over sodium sulfate, filtered, concentrated in vacuo, and purified via silica gel chromatography (Gradient: 0% to 5% methanol in dichloromethane), affording C132 as a yellow solid. Yield: 1.60 g, 6.24 mmol, 96%. LCMS m/z 257.1 [M+H]⁺. ¹H NMR (400 MHz, DMSO-d₆) δ 10.45 (s, 1H), 8.71 (s, 1H), 8.03 (s, 1H), 7.59 (d, half of AB quartet, J=9.3 Hz, 1H), 7.55 (dd, component of ABX system, J=9.4, 1.4 Hz, 1H), 6.77 (dd, J=17.6, 11.0 Hz, 1H), 5.92 (d, J=17.7 Hz, 1H), 5.34 (d, J=11.1 Hz, 1H), 3.78 (t, J=6.7 Hz, 2H), 2.78 (t, J=6.7 Hz, 2H).

Step 2. Synthesis of 3-(2,4-dioxo-1,3-diazinan-1-yl)pyrazolo[1,5-a]pyridine-6-carbaldehyde (P248): To a solution of C132 (1.60 g, 6.24 mmol) in a mixture of tetrahydrofuran (50 mL) and water (10 mL) were added potassium osmate(VI) dihydrate (207 mg, 0.562 mmol) and 4-methylmorpholine N-oxide (1.46 g, 12.5 mmol). After the reaction mixture had been stirred at 25° C. for 16 hours, sodium periodate (4.01 g, 18.7 mmol) was added, and stirring was continued at 25° C. for 2 hours. Dilution with water was followed by extraction with ethyl acetate (2×100 mL); the combined organic layers were washed with aqueous sodium thiosulfate solution (100 mL), dried over sodium sulfate, filtered, and concentrated in vacuo. Purification using chromatography on silica gel (Gradient: 0% to 60% ethyl acetate in petroleum ether) afforded P248 as a yellow solid. Yield: 500 mg, 1.94 mmol, 31%. LCMS m/z 259.1 [M+H]⁺. ¹H NMR (400 MHz, DMSO-d₆) δ 10.54 (s, 1H), 9.94 (br s, 1H), 9.49-9.45 (m, 1H), 8.34 (s, 1H), 7.70 (br d, half of AB quartet, J=9.3 Hz, 1H), 7.52 (dd, component of ABX system, J=9.3, 1.4 Hz, 1H), 3.82 (t, J=6.7 Hz, 2H), 2.79 (t, J=6.7 Hz, 2H).

### Preparation P249

### Pentafluorophenyl 4-chloro-3-(2,4-dioxo-1,3-diazinan-1-yl)benzoate (P249)

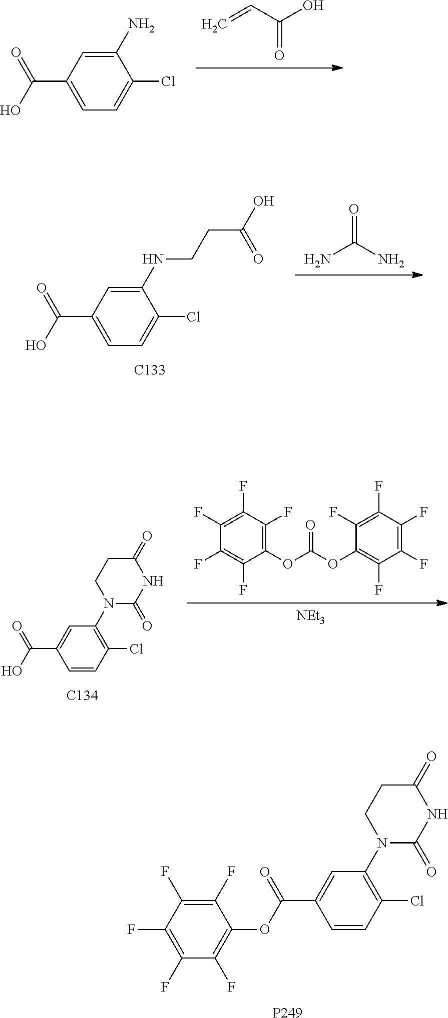

Step 1. Synthesis of 3-[(2-carboxyethyl)amino]-4-chlorobenzoic acid (C133): A mixture of 3-amino-4-chlorobenzoic acid (10.0 g, 58.3 mmol) and prop-2-enoic acid (100 mL) was stirred at 100° C. for 3 hours. The reaction mixture was diluted with water (200 mL) and the solid was collected via filtration, affording C133 as an off-white solid. Yield: 14.0 g, 57.5 mmol, 99%. LCMS m/z 244.1 (chlorine isotope pattern observed) [M+H]⁺. ¹H NMR (400 MHz, DMSO-d₆) δ 12.65 (br s, 2H), 7.37 (d, half of AB quartet, J=8.1 Hz, 1H), 7.23 (d, half of AC quartet, J=1.9 Hz, 1H), 7.17 (dd, component of ABC system, J=8.1, 1.9 Hz, 1H), 5.57 (t, J=5.8 Hz, 1H), 3.43-3.35 (m, 2H), 2.57 (t, J=6.9 Hz, 2H).

Step 2. Synthesis of 4-chloro-3-(2,4-dioxo-1,3-diazinan-1-yl)benzoic acid (C134): To a solution of C133 (14.0 g, 57.5 mmol) in acetic acid (200 mL) was added urea (34.5 g, 574 mmol). The reaction mixture was stirred at 100° C. for 3 days, whereupon it was combined with a similar reaction carried out using C133 (820 mg, 3.36 mmol), concentrated in vacuo, and diluted with water (500 mL). Filtration provided a filter cake, which was stirred with methyl tert-butyl ether (300 mL) at 25° C. for 10 minutes; a second filtration afforded C134 as an off-white solid. Combined yield: 11.0 g, 40.9 mmol, 67%. LCMS m/z 269.1 (chlorine isotope pattern observed) [M+H]⁺. ¹H NMR (400 MHz, DMSO-d₆) δ 10.52 (s, 1H), 8.04 (d, half of AB quartet, J=2.0 Hz, 1H), 7.90 (dd, component of ABC system, J=8.4, 2.1 Hz, 1H), 7.70 (d, half of AC quartet, J=8.4 Hz, 1H), 3.84-3.72 (m, 1H), 3.67-3.56 (m, 1H), 2.86-2.64 (m, 2H).

Step 3. Synthesis of pentafluorophenyl 4-chloro-3-(2,4-dioxo-1,3-diazinan-1-yl)benzoate (P249): Triethylamine (1.13 g, 11.2 mmol) was added drop-wise to a suspension of C134 (2.00 g, 7.44 mmol) and bis(pentafluorophenyl) carbonate (2.93 g, 7.43 mmol) in acetonitrile (20 mL). After the reaction mixture had been stirred at 20° C. for 2 hours, it was concentrated in vacuo and partitioned between dichloromethane (60 mL) and water (40 mL). The aqueous layer was extracted with dichloromethane (50 mL), and the combined organic layers were washed with saturated aqueous sodium chloride solution (50 mL), dried over sodium sulfate, filtered, and concentrated in vacuo. Silica gel chromatography (Eluent: 7:10 ethyl acetate/petroleum ether) provided P249 as a white solid. Yield: 2.85 g, 6.56 mmol, 88%. LCMS m/z 435.0 (chlorine isotope pattern observed) [M+H]⁺. ¹H NMR (400 MHz, DMSO-d₆) δ 10.52 (s, 1H), 8.04 (d, J=2.1 Hz, 1H), 7.90 (dd, component of ABC system, J=8.3, 2.1 Hz, 1H), 7.71 (d, half of AB quartet, J=8.4 Hz, 1H), 3.85-3.72 (m, 1H), 3.69-3.56 (m, 1H), 2.86-2.66 (m, 2H).

### Preparation P250

### N-{[(1r,4r)-4-(5-Chloro-2H-pyrazolo[3,4-c]pyridin-2-yl)cyclohexyl]methyl}-3,5-difluoro-4-[(4-methoxyphenyl)methoxy]benzamide (P250)

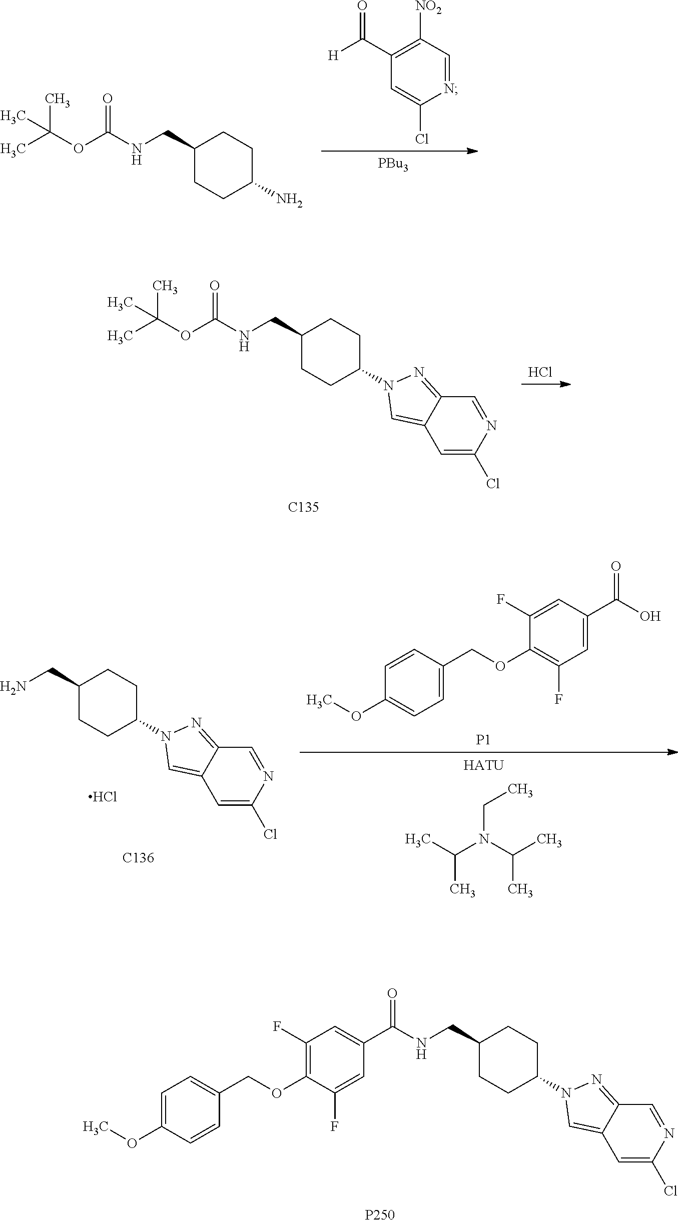

Step 1. Synthesis of tert-butyl {[(1r,4r)-4-(5-chloro-2H-pyrazolo[3,4-c]pyridin-2-yl)cyclohexyl]methyl}carbamate (C135): A suspension of 2-chloro-5-nitropyridine-4-carbaldehyde (2.80 g, 15.0 mmol) and tert-butyl {[(1r,4r)-4-aminocyclohexyl]methyl}carbamate (3.77 g, 16.5 mmol) in propan-2-ol (30 mL) was heated at 80° C. for 4 hours. The reaction mixture was cooled to room temperature and treated with tributylphosphine (6.07 g, 30.0 mmol), whereupon it was heated at 80° C. overnight. Concentration in vacuo was followed by silica gel chromatography (Eluent: 2:5 ethyl acetate/petroleum ether), affording C135 as a yellow solid. Yield: 3.20 g, 8.77 mmol, 58%. LCMS m/z 365.2 (chlorine isotope pattern observed) [M+H]⁺. ¹H NMR (400 MHz, chloroform-d) δ 9.07-9.04 (m, 1H), 7.96 (br s, 1H), 7.56 (d, J=1.3 Hz, 1H), 4.70-4.58 (m, 1H), 4.45 (tt, J=12.0, 3.8 Hz, 1H), 3.07 (t, J=6.5 Hz, 2H), 2.37-2.28 (m, 2H), 2.08-1.90 (m, 4H), 1.70-1.56 (m, 1H), 1.46 (s, 9H), 1.31-1.16 (m, 2H).

Step 2. Synthesis of 1-[(1r,4r)-4-(5-chloro-2H-pyrazolo[3,4-c]pyridin-2-yl)cyclohexyl]methanamine, hydrochloride salt (C136): A solution of hydrogen chloride in 1,4-dioxane (4 M; 20 mL, 80 mmol) was added to a solution of C135 (5.00 g, 13.7 mmol) in methanol (30 mL). After the reaction mixture had been stirred at room temperature for 2 hours, LCMS analysis indicated conversion to C136: LCMS m/z 265.2 (chlorine isotope pattern observed) [M+H]⁺. Concentration in vacuo provided C136 as a gray solid. Yield: 2.71 g, 9.00 mmol, 66%.

Step 3. Synthesis of N-{[(1r,4r)-4-(5-chloro-2H-pyrazolo[3,4-c]pyridin-2-yl)cyclohexyl]methyl}-3,5-difluoro-4-[(4-methoxyphenyl)methoxy]benzamide (P250): N,N-Diisopropylethylamine (3.48 g, 26.9 mmol) was added to a solution of P1 (3.44 g, 11.7 mmol) and O-(7-azabenzotriazol-1-yl)-N,N,N′N′-tetramethyluronium hexafluorophosphate (HATU; 5.13 g, 13.5 mmol) in N,N-dimethylformamide (50.0 mL). After the resulting mixture had been stirred for 5 minutes, C136 (2.71 g, 9.00 mmol) was added, and stirring was continued at room temperature for 2 hours. The reaction mixture was then diluted with water, and the solid was collected via filtration and washed three times with water, affording P250 as a gray solid. Yield: 4.80 g, 8.87 mmol, 99%. LCMS m/z 541.2 (chlorine isotope pattern observed) [M+H]⁺. ¹H NMR (400 MHz, DMSO-d₆) δ 9.02 (t, J=1.1 Hz, 1H), 8.61-8.55 (m, 2H), 7.79 (d, J=1.2 Hz, 1H), 7.66-7.56 (m, 2H), 7.34 (d, J=8.7 Hz, 2H), 6.92 (d, J=8.7 Hz, 2H), 5.17 (s, 2H), 4.67-4.56 (m, 1H), 3.74 (s, 3H), 3.18 (t, J=6.3 Hz, 2H), 2.21-2.10 (m, 2H), 1.99-1.83 (m, 4H), 1.74-1.60 (m, 1H), 1.30-1.14 (m, 2H).

### Preparation P251

### 2,3,5-Trifluoro-4-[(4-methoxyphenyl)methoxy]-N-({4-[6-(piperazin-1-yl)-2H-indazol-2-yl]bicyclo[2.2.2]octan-1-yl}methyl)benzamide (P251)

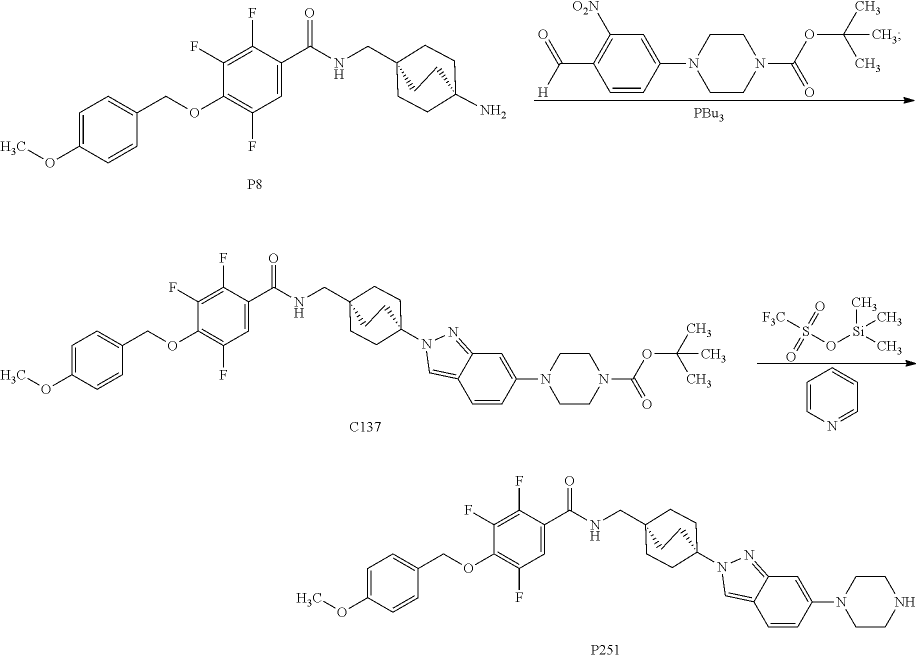

Step 1. Synthesis of tert-butyl 4-{2-[4-({2,3,5-trifluoro-4-[(4-methoxyphenyl)methoxy]benzamido}methyl)bicyclo[2.2.2]octan-1-yl]-2H-indazol-6-yl}piperazine-1-carboxylate (C137): A mixture of P8 (1.40 g, 3.12 mmol) and tert-butyl 4-(4-formyl-3-nitrophenyl)piperazine-1-carboxylate (1.36 g, 4.06 mmol) in propan-2-ol (20 mL) was stirred at 85° C. for 4 hours, whereupon it was cooled to 20° C. Tributylphosphine (2.53 g, 12.5 mmol) was added, and the reaction mixture was heated at 85° C. for 16 hours. After removal of solvent via concentration in vacuo, the residue was purified using silica gel chromatography (Gradient: 20% to 50% ethyl acetate in petroleum ether), affording C137 as a yellow solid. Yield: 670 mg, 0.913 mmol, 29%. LCMS m/z 734.3 [M+H]⁺. ¹H NMR (400 MHz, DMSO-d₆), aliphatic integrations are approximate: δ 8.39 (br t, J=6 Hz, 1H), 8.18 (s, 1H), 7.50 (d, J=9.1 Hz, 1H), 7.37-7.31 (m, 1H), 7.36 (d, J=8.6 Hz, 2H), 6.94 (d, J=8.7 Hz, 2H), 6.89 (dd, J=9.1, 2.0 Hz, 1H), 6.81 (br s, 1H), 5.22 (s, 2H), 3.75 (s, 3H), 3.52-3.42 (m, 4H), 3.09 (d, J=6.3 Hz, 2H), 3.07-3.01 (m, 4H), 2.16-2.06 (m, 6H), 1.69-1.59 (m, 6H), 1.42 (s, 9H).

Step 2. Synthesis of 2,3,5-trifluoro-4-[(4-methoxyphenyl)methoxy]-N-({4-[6-(piperazin-1-yl)-2H-indazol-2-yl]bicyclo[2.2.2]octan-1-yl}methyl)benzamide (P251): Trimethylsilyl trifluoromethanesulfonate (1.22 g, 5.49 mmol) was added to a solution of C137 (670 mg, 0.913 mmol) and pyridine (506 mg, 6.40 mmol) in dichloromethane (15 mL), and the reaction mixture was stirred at 20° C. for 2 hours. Aqueous sodium carbonate solution (2 M; 50 mL) was then added, and the mixture was extracted with dichloromethane (2×40 mL); the combined organic layers were washed with saturated aqueous sodium chloride solution (20 mL), dried over sodium sulfate, filtered, and concentrated in vacuo. Silica gel chromatography (Gradient: 9% to 23% methanol in dichloromethane) provided P251 as a yellow solid. Yield: 420 mg, 0.663 mmol, 73%. LCMS m/z 634.3 [M+H]⁺. ¹H NMR (400 MHz, DMSO-d₆) δ 8.41 (br t, J=6 Hz, 1H), 8.16 (s, 1H), 7.47 (d, J=9.1 Hz, 1H), 7.40-7.30 (m, 3H), 6.94 (d, J=8.5 Hz, 2H), 6.88 (br d, J=9.2 Hz, 1H), 6.75 (br s, 1H), 5.22 (s, 2H), 3.75 (s, 3H), 3.43-3.27 (m, 4H), 3.09 (d, J=6.2 Hz, 2H), 3.06-2.98 (m, 4H), 2.16-2.06 (m, 6H), 1.69-1.58 (m, 6H).

### Preparation P252

### 2,3,5-Trifluoro-N-({(1r,4r)-4-[6-(N-hydroxycarbamimidoyl)-2H-indazol-2-yl]cyclohexyl}methyl)-4-[(4-methoxyphenyl)methoxy]benzamide (P252)

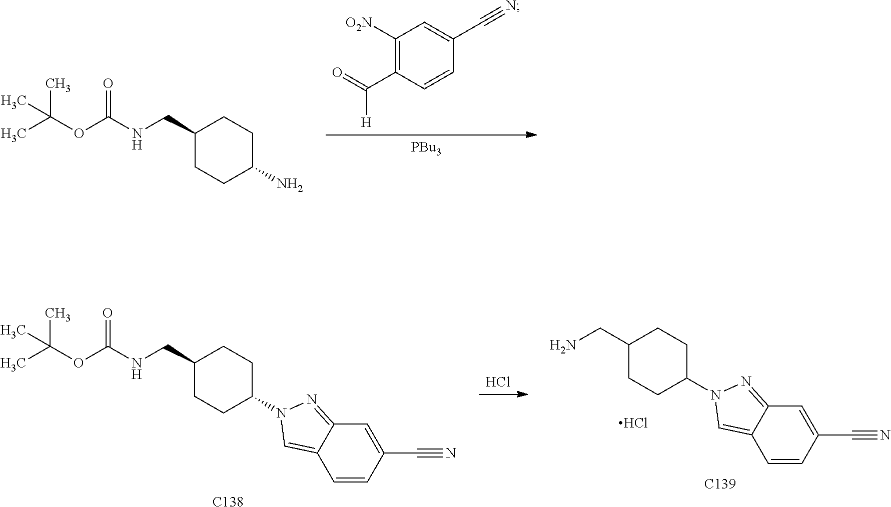

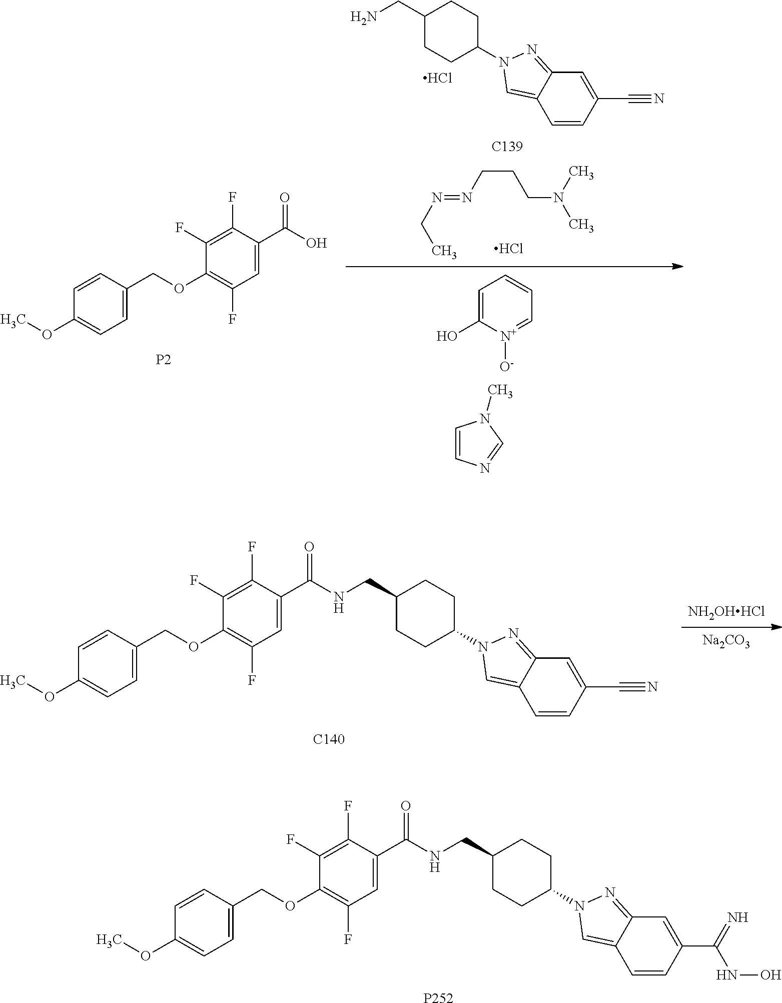

Step 1. Synthesis of tert-butyl {[(1r,4r)-4-(6-cyano-2H-indazol-2-yl)cyclohexyl]methyl}carbamate (C138): A solution of 4-formyl-3-nitrobenzonitrile (3.00 g, 17.0 mmol) and tert-butyl {[(1r,4r)-4-aminocyclohexyl]methyl}carbamate (3.89 g, 17.0 mmol) in propan-2-ol (30 mL) was stirred at 85° C. for 4 hours, whereupon it was cooled to room temperature and treated with tributylphosphine (6 mL, 24.1 mmol). After the reaction mixture had been stirred at 85° C. overnight, LCMS analysis indicated conversion to C138: LCMS m/z 377.2 [M+Na⁺]. The reaction mixture was combined with a similar reaction carried out using 4-formyl-3-nitrobenzonitrile (1.00 g, 5.68 mmol), concentrated in vacuo, and purified via chromatography on silica gel (Eluent: 4% methanol in dichloromethane) to provide C138 as a yellow solid. Combined yield: 4.10 g, 11.6 mmol, 51%. ¹H NMR (400 MHz, DMSO-d₆) δ 8.61 (br s, 1H), 8.31-8.29 (m, 1H), 7.90 (dd, J=8.6, 1.0 Hz, 1H), 7.26 (dd, J=8.7, 1.3 Hz, 1H), 6.90 (br t, J=6 Hz, 1H), 4.54 (tt, J=11.6, 3.6 Hz, 1H), 2.85 (t, J=6.3 Hz, 2H), 2.19-2.09 (m, 2H), 1.96-1.79 (m, 4H), 1.54-1.43 (m, 1H), 1.39 (s, 9H), 1.21-1.06 (m, 2H).

Step 2. Synthesis of 2-[4-(aminomethyl)cyclohexyl]-2H-indazole-6-carbonitrile, hydrochloride salt (C139): A solution of hydrogen chloride in 1,4-dioxane (4 M; 3 mL, 12 mmol) was added to a solution of C138 (1.0 g, 2.8 mmol) in dichloromethane (15 mL), and the reaction mixture was stirred at 25° C. for 2 hours. After concentration in vacuo, the residue was triturated with ethyl acetate, affording C139 as a light-yellow solid. Yield: 660 mg, 2.27 mmol, 81%. LCMS m/z 255.2 [M+H]⁺. ¹H NMR (400 MHz, DMSO-d₆) δ 8.65 (d, J=1.0 Hz, 1H), 8.32-8.29 (m, 1H), 8.12 (br s, 3H), 7.91 (dd, J=8.6, 1.0 Hz, 1H), 7.27 (dd, J=8.6, 1.3 Hz, 1H), 4.62-4.52 (m, 1H), 2.77-2.67 (m, 2H), 2.23-2.12 (m, 2H), 2.04-1.84 (m, 4H), 1.80-1.66 (m, 1H), 1.30-1.15 (m, 2H).

Step 3. Synthesis of N-{[(1r,4r)-4-(6-cyano-2H-indazol-2-yl)cyclohexyl]methyl}-2,3,5-trifluoro-4-[(4-methoxyphenyl)methoxy]benzamide (C140): To a solution of P2 (800 mg, 2.56 mmol), C139 (650 mg, 2.24 mmol), and 2-hydroxypyridine 1-oxide (399 mg, 3.59 mmol) in N,N-dimethylformamide (20 mL) was added 1-methyl-1H-imidazole (841 mg, 10.2 mmol). After the mixture had been stirred at 25° C. for 2 minutes, 1-[3-(dimethylamino)propyl]-3-ethylcarbodiimide hydrochloride (589 mg, 3.07 mmol) was added, and stirring was continued at 25° C. for 2 hours. Water (100 mL) was added and the resulting mixture was extracted with ethyl acetate (2×100 mL); the combined organic layers were washed with saturated aqueous sodium chloride solution, dried over sodium sulfate, filtered, and concentrated in vacuo. Chromatography on silica gel (Eluent: 2:1 petroleum ether/ethyl acetate) provided C140 as a white solid. Yield: 1.10 g, 2.01 mmol, 90%. LCMS m/z 549.2 [M+H]⁺. ¹H NMR (400 MHz, DMSO-d₆) δ 8.62 (br s, 1H), 8.51 (br t, J=6 Hz, 1H), 8.32-8.30 (m, 1H), 7.90 (dd, J=8.6, 1.0 Hz, 1H), 7.39-7.33 (m, 1H), 7.35 (d, J=8.6 Hz, 2H), 7.27 (dd, J=8.6, 1.3 Hz, 1H), 6.94 (d, J=8.6 Hz, 2H), 5.22 (s, 2H), 4.64-4.50 (m, 1H), 3.75 (s, 3H), 3.17 (t, J=6.3 Hz, 2H), 2.22-2.10 (m, 2H), 1.98-1.84 (m, 4H), 1.73-1.59 (m, 1H), 1.30-1.15 (m, 2H).

Step 4. Synthesis of 2,3,5-trifluoro-N-({(1r,4r)-4-[6-(N-hydroxycarbamimidoyl)-2H-indazol-2-yl]cyclohexyl}methyl)-4-[(4-methoxyphenyl)methoxy]benzamide (P252): A mixture of C140 (350 mg, 0.638 mmol), hydroxylamine hydrochloride (53.2 mg, 0.766 mmol), and sodium carbonate (271 mg, 2.56 mmol) in ethanol (10 mL) was stirred at 85° C. for 12 hours, whereupon the reaction mixture was concentrated in vacuo. The residue was triturated with water (20 mL) at 25° C. for 10 minutes, providing P252 as a white solid. Yield: 350 mg, 0.602 mmol, 94%. LCMS m/z 582.3 [M+H]⁺. ¹H NMR (400 MHz, DMSO-d₆) δ 9.67 (br s, 1H), 8.55 (br t, J=6 Hz, 1H), 8.38 (s, 1H), 7.90 (s, 1H), 7.60 (d, J=8.8 Hz, 1H), 7.42 (dd, J=8.8, 1.4 Hz, 1H), 7.40-7.34 (m, 1H), 7.35 (d, J=8.6 Hz, 2H), 6.94 (d, J=8.7 Hz, 2H), 5.83 (br s, 2H), 5.22 (s, 2H), 4.52-4.40 (m, 1H), 3.75 (s, 3H), 3.16 (t, J=6.3 Hz, 2H), 2.20-2.09 (m, 2H), 1.98-1.81 (m, 4H), 1.73-1.59 (m, 1H), 1.30-1.13 (m, 2H).

In the IUPAC names for the following Examples, the stereochemistry of the 2,6-dioxopiperidine moiety has been indicated as (3RS), to emphasize that these compounds are racemic at that 3-position. Other stereocenters that are drawn with a straight bond should also be understood as representing an equal mixture of both possible stereochemistries at that center.

### Example 1

### N-{[(1r,4r)-4-{6-[2-(3-{1-[(3RS)-2,6-Dioxopiperidin-3-yl]-3-methyl-2-oxo-2,3-dihydro-1H-benzimidazol-5-yl}propyl)-2,3-dihydro-1H-isoindol-5-yl]-2H-indazol-2-yl}cyclohexyl]methyl}-2,3,5-trifluoro-4-hydroxybenzamide, hydrochloride salt (1)

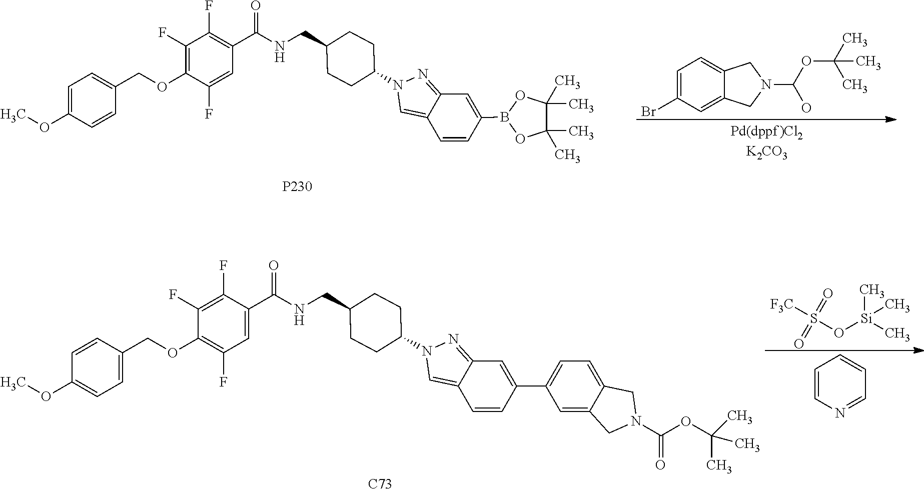

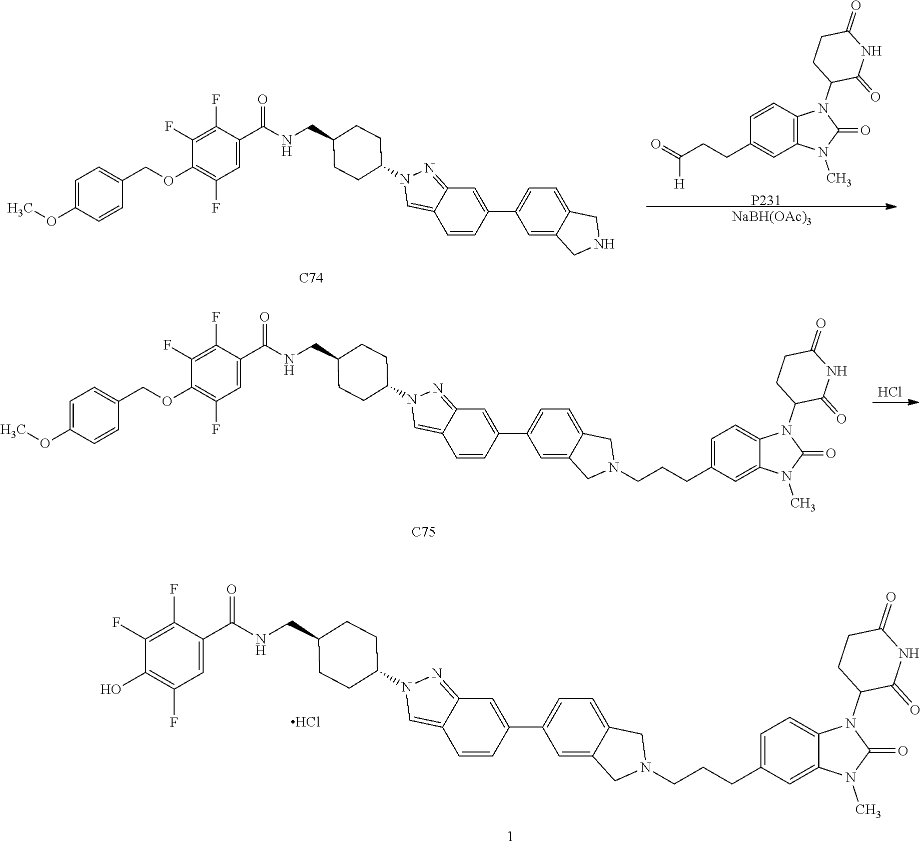

Step 1. Synthesis of tert-butyl 5-{2-[(1r,4r)-4-({2,3,5-trifluoro-4-[(4-methoxyphenyl)methoxy]benzamido}methyl)cyclohexyl]-2H-indazol-6-yl}-1,3-dihydro-2H-isoindole-2-carboxylate (C73): A solution of P230 (2.50 g, 3.85 mmol), tert-butyl 5-bromo-1,3-dihydro-2H-isoindole-2-carboxylate (1.26 g, 4.23 mmol), potassium carbonate (1.06 g, 7.67 mmol), and [1,1′-bis(diphenylphosphino)ferrocene]dichloropalladium(II) (310 mg, 0.424 mmol) in a mixture of 1,4-dioxane (30 mL) and water (6 mL) was stirred at 90° C. for 2 hours. After concentration of the reaction mixture under reduced pressure, silica gel chromatography (Gradient: 0% to 10% methanol in dichloromethane) afforded C73 as a yellow solid. Yield: 2.32 g, 3.13 mmol, 81%. ¹H NMR (400 MHz, chloroform-d) δ 7.96 (s, 1H), 7.87 (br s, 1H), 7.71 (d, J=8.6 Hz, 1H), [7.63-7.53 (m) and 7.50 (s), total 3H], 7.38-7.28 (m, 4H), 6.88 (d, J=8.6 Hz, 2H), 6.72-6.62 (m, 1H), 5.24 (s, 2H), 4.79-4.66 (m, 4H), 4.50-4.37 (m, 1H), 3.80 (s, 3H), 3.42 (t, J=6.1 Hz, 2H), 2.42-2.30 (m, 2H), 2.12-1.92 (m, 4H), 1.87-1.72 (m, 1H), 1.53 (s, 9H), 1.38-1.24 (m, 2H).

Step 2. Synthesis of N-({(1r,4r)-4-[6-(2,3-dihydro-1H-isoindol-5-yl)-2H-indazol-2-yl]cyclohexyl}methyl)-2,3,5-trifluoro-4-[(4-methoxyphenyl)methoxy]benzamide (C74): Trimethylsilyl trifluoromethanesulfonate (0.362 mL, 2.00 mmol) was added to a 0° C. solution of C73 (371 mg, 0.501 mmol) and pyridine (0.323 mL, 3.99 mmol) in dichloromethane (17 mL), whereupon the reaction mixture was allowed to slowly warm to room temperature and stir overnight. LCMS analysis at this point indicated conversion to C74: LCMS m/z 641.6 [M+H]⁺. After the reaction mixture had been cooled to 0° C., it was treated with saturated aqueous sodium bicarbonate solution, and the aqueous layer was extracted three times with dichloromethane. The combined organic layers were washed with saturated aqueous sodium chloride solution, dried over sodium sulfate, filtered, and concentrated in vacuo to provide C74; this material was progressed directly to the following step. LCMS m/z 641.6 [M+H]⁺. ¹H NMR (400 MHz, DMSO-d₆) δ 8.49 (br t, J=6 Hz, 1H), 8.39 (s, 1H), 7.73 (d, J=8.8 Hz, 1H), 7.56 (s, 1H), 7.50 (br d, J=7.8 Hz, 1H), 7.41-7.28 (m, 5H), 6.93 (d, J=8.6 Hz, 2H), 5.21 (s, 2H), 4.57-4.41 (m, 2H), 4.16-4.05 (m, 4H), 3.74 (s, 3H), 3.17 (t, J=6.3 Hz, 2H), 2.22-2.10 (m, 2H), 2.00-1.83 (m, 4H), 1.74-1.58 (m, 1H), 1.31-1.13 (m, 2H).

Step 3. Synthesis of N-({(1r,4r)-4-[6-(2-{3-[1-(2,6-dioxopiperidin-3-yl)-3-methyl-2-oxo-2,3-dihydro-1H-benzimidazol-5-yl]propyl}-2,3-dihydro-1H-isoindol-5-yl)-2H-indazol-2-yl]cyclohexyl}methyl)-2,3,5-trifluoro-4-[(4-methoxyphenyl)methoxy]benzamide (C75): A mixture of C74 (from the previous step; 50.501 mmol) and P231 (90%, 193 mg, 0.551 mmol) in tetrahydrofuran (8.4 mL) was stirred at room temperature for 30 minutes, whereupon sodium triacetoxyborohydride (425 mg, 2.00 mmol) was added. After the reaction mixture had been heated at 50° C. for 30 minutes, LCMS analysis indicated conversion to C75: LCMS m/z 940.8 [M+H]⁺. The reaction mixture was diluted with dichloromethane and treated with aqueous sodium bicarbonate solution, and the aqueous layer was extracted three times with dichloromethane. The combined organic layers were washed with saturated aqueous sodium chloride solution, dried over sodium sulfate, filtered, and concentrated in vacuo; chromatography using neutral alumina (Gradient: 0% to 15% methanol in ethyl acetate) afforded C75 as a pale-red solid, which was used directly in the following step. Yield: 224 mg, 0.238 mmol, 48% over 2 steps. ¹H NMR (400 MHz, DMSO-d₆), characteristic peaks: δ 11.09 (s, 1H), 8.50 (br t, J=6 Hz, 1H), 8.41 (s, 1H), 7.78 (br s, 1H), 7.75 (d, J=8.7 Hz, 1H), 7.56 (s, 1H), 7.53 (br d, J=7.8 Hz, 1H), 7.40-7.29 (m, 5H), 7.09 (br s, 1H), 7.02 (d, J=8.0 Hz, 1H), 6.97-6.90 (m, 3H), 5.35 (dd, J=12.7, 5.4 Hz, 1H), 5.22 (s, 2H), 4.53-4.42 (m, 1H), 3.95-3.86 (m, 4H), 3.75 (s, 3H), 3.34 (s, 3H), 3.18 (t, J=6.3 Hz, 2H), 2.22-2.12 (m, 2H), 2.06-1.80 (m, 7H), 1.73-1.60 (m, 1H), 1.30-1.16 (m, 2H).

Step 4. Synthesis of N-{[(1r,4r)-4-{6-[2-(3-{1-[(3RS)-2,6-dioxopiperidin-3-yl]-3-methyl-2-oxo-2,3-dihydro-1H-benzimidazol-5-yl}propyl)-2,3-dihydro-1H-isoindol-5-yl]-2H-indazol-2-yl}cyclohexyl]methyl}-2,3,5-trifluoro-4-hydroxybenzamide, hydrochloride salt (1): A solution of hydrogen chloride in 1,4-dioxane (4 M; 1.97 mL, 7.88 mmol) was added drop-wise to a 0° C. solution of C75 (224 mg, 0.238 mmol) in dichloromethane (4.8 mL), whereupon the reaction mixture was removed from the ice bath and allowed to stir at room temperature for 2 hours. A sticky brown solid was collected via filtration; this was stirred with acetonitrile (4 mL) for 30 minutes at room temperature, collected via filtration, and suspended in propan-2-ol (4 mL). After this mixture had been stirred for 45 minutes, filtration afforded N-{[(1r,4r)-4-{6-[2-(3-{1-[(3RS)-2,6-dioxopiperidin-3-yl]-3-methyl-2-oxo-2,3-dihydro-1H-benzimidazol-5-yl}propyl)-2,3-dihydro-1H-isoindol-5-yl]-2H-indazol-2-yl}cyclohexyl]methyl}-2,3,5-trifluoro-4-hydroxybenzamide, hydrochloride salt (1) as a pale-brown solid. Yield: 56 mg, 65 μmol, 27%. LCMS m/z 820.7 [M+H]⁺. ¹H NMR (400 MHz, DMSO-d₆) δ 11.36 (br s, 2H), 11.10 (s, 1H), 8.45 (s, 1H), 8.35 (br t, J=6 Hz, 1H), 7.85 (br s, 1H), 7.79 (d, J=8.7 Hz, 1H), 7.76-7.71 (m, 2H), 7.49 (d, J=7.9 Hz, 1H), 7.34 (dd, J=8.7, 1.6 Hz, 1H), 7.33-7.28 (m, 1H), 7.13 (br s, 1H), 7.07 (d, J=8.0 Hz, 1H), 6.96 (br d, J=8.2 Hz, 1H), 5.37 (dd, J=12.7, 5.4 Hz, 1H), 4.92-4.81 (m, 2H), 4.62-4.45 (m, 3H), 3.46-3.36 (m, 2H), 3.36 (s, 3H), 3.19 (t, J=6.3 Hz, 2H), 2.98-2.86 (m, 1H), 2.81-2.58 (m, 4H), 2.23-2.06 (m, 4H), 2.06-1.86 (m, 5H), 1.76-1.62 (m, 1H), 1.31-1.18 (m, 2H).

### Example 2

### N-{[(1r,4r)-4-{6-[6-(3-{1-[(3RS)-2,6-Dioxopiperidin-3-yl]-3-methyl-2-oxo-2,3-dihydro-1H-benzimidazol-5-yl}propyl)-6,7-dihydro-5H-pyrrolo[3,4-b]pyridin-2-yl]-2H-indazol-2-yl}cyclohexyl]methyl}-2,3,5-trifluoro-4-hydroxybenzamide (2)

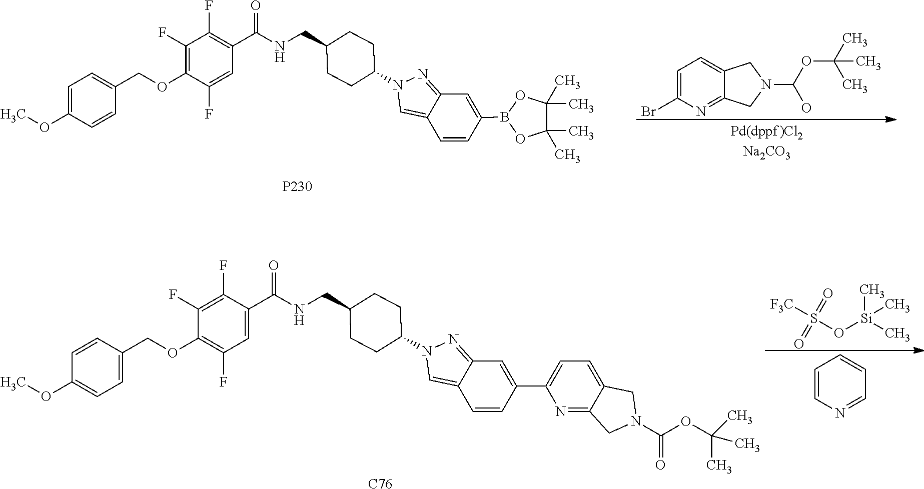

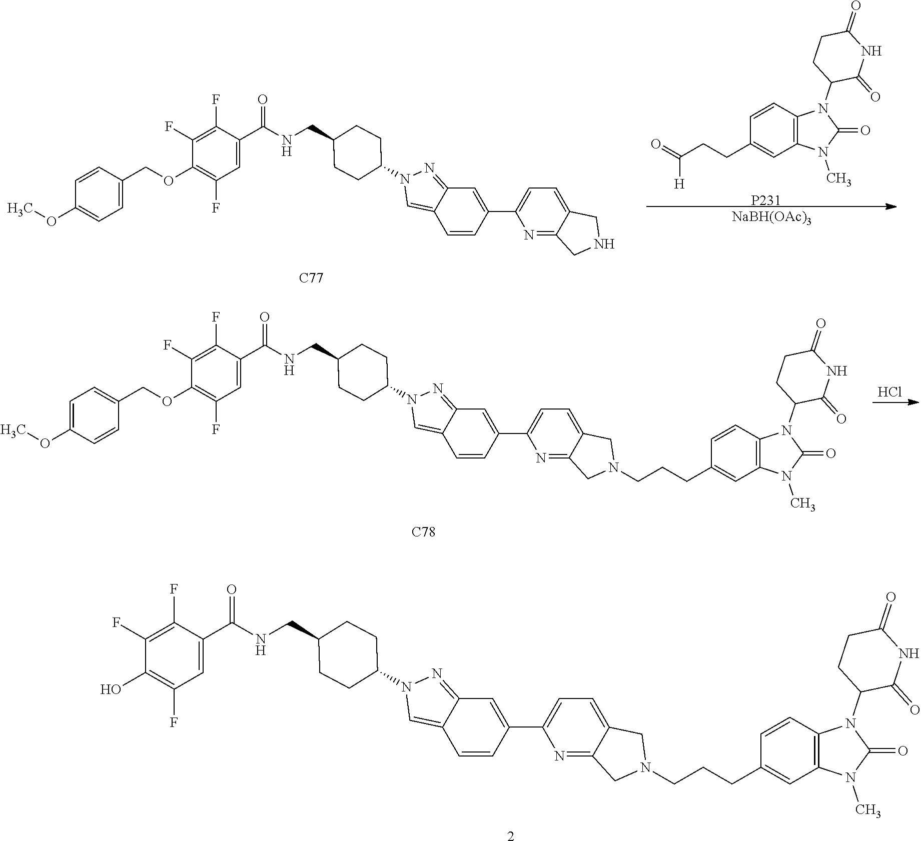

Step 1. Synthesis of tert-butyl 2-{2-[(1r,4r)-4-({2,3,5-trifluoro-4-[(4-methoxyphenyl)methoxy]benzamido}methyl)cyclohexyl]-2H-indazol-6-yl}-5,7-dihydro-6H-pyrrolo[3,4-b]pyridine-6-carboxylate (C76): A solution of P230 (597 mg, 0.919 mmol), tert-butyl 2-bromo-5,7-dihydro-6H-pyrrolo[3,4-b]pyridine-6-carboxylate (250 mg, 0.836 mmol), sodium carbonate (221 mg, 2.09 mmol), and [1,1′-bis(diphenylphosphino)ferrocene]dichloropalladium(II) (91.7 mg, 0.125 mmol) in a mixture of 1,4-dioxane (30 mL) and water (3 mL) was stirred for 16 hours at 80° C. The reaction mixture was then concentrated in vacuo, and the residue was purified via chromatography on silica gel (Gradient: 0% to 100% ethyl acetate in petroleum ether), affording C76 as a light-yellow solid. Yield: 320 mg, 0.431 mmol, 52%. LCMS m/z 742.4 [M+H]⁺. ¹H NMR (400 MHz, DMSO-d₆) δ 8.51 (br t, J=6 Hz, 1H), 8.43 (s, 1H), 8.29 (s, 1H), 7.97 (d, half of AB quartet, J=8.1 Hz, 1H), 7.89-7.73 (m, 3H), 7.43-7.32 (m, 1H), 7.36 (d, J=8.3 Hz, 2H), 6.94 (d, J=8.3 Hz, 2H), 5.22 (s, 2H), 4.71-4.59 (m, 4H), 4.56-4.44 (m, 1H), 3.75 (s, 3H), 3.18 (br t, J=6 Hz, 2H), 2.24-2.13 (m, 2H), 2.03-1.84 (m, 4H), 1.74-1.59 (m, 1H), 1.48 (s, 9H), 1.32-1.14 (m, 2H).

Step 2. Synthesis of N-({(1r,4r)-4-[6-(6,7-dihydro-5H-pyrrolo[3,4-b]pyridin-2-yl)-2H-indazol-2-yl]cyclohexyl}methyl)-2,3,5-trifluoro-4-[(4-methoxyphenyl)methoxy]benzamide (C77): Trimethylsilyl trifluoromethanesulfonate (240 mg, 1.08 mmol) was added drop-wise to a 0° C. mixture of C76 (200 mg, 0.270 mmol) and pyridine (171 mg, 2.16 mmol) in dichloromethane (15 mL). After the reaction mixture had been stirred at 25° C. for 16 hours, it was combined with a similar reaction carried out using C76 (120 mg, 0.162 mmol), and treated with aqueous sodium carbonate solution (40 mL) and aqueous sodium bicarbonate solution (40 mL). The resulting mixture was extracted with dichloromethane (2×30 mL); the combined organic layers were washed with saturated aqueous sodium chloride solution, filtered, dried, and concentrated in vacuo. Silica gel chromatography (Gradient: 0% to 100% ethyl acetate in petroleum ether, followed by 0% to 20% methanol in dichloromethane) provided C77 as a pale-yellow solid. Combined yield: 160 mg, 0.249 mmol, 58%. LCMS m/z 642.3 [M+H]⁺. ¹H NMR (400 MHz, DMSO-d₆) δ 8.51 (br t, J=6 Hz, 1H), 8.44 (s, 1H), 8.30-8.26 (m, 1H), [7.97 (d, half of AB quartet, J=8.1 Hz) and 7.91 (d, half of AB quartet, J=8.0 Hz), total 1H], 7.87-7.74 (m, 3H), 7.41-7.32 (m, 1H), 7.36 (d, J=8.7 Hz, 2H), 6.94 (d, J=8.6 Hz, 2H), 5.22 (s, 2H), 4.66 (br d, J=9.0 Hz, 2H), 4.56-4.44 (m, 1H), 4.31 (d, J=18.7 Hz, 2H), 3.75 (s, 3H), 3.18 (t, J=6.3 Hz, 2H), 2.24-2.12 (m, 2H), 2.02-1.85 (m, 4H), 1.74-1.60 (m, 1H), 1.32-1.16 (m, 2H).

Step 3. Synthesis of N-({(1r,4r)-4-[6-(6-{3-[1-(2,6-dioxopiperidin-3-yl)-3-methyl-2-oxo-2,3-dihydro-1H-benzimidazol-5-yl]propyl}-6,7-dihydro-5H-pyrrolo[3,4-b]pyridin-2-yl)-2H-indazol-2-yl]cyclohexyl}methyl)-2,3,5-trifluoro-4-[(4-methoxyphenyl)methoxy]benzamide (C78): To a 0° C. solution of C77 (160 mg, 0.249 mmol) and P231 (82.6 mg, 0.262 mmol) in a mixture of dimethyl sulfoxide (4 mL) and dichloromethane (20 mL) was added sodium triacetoxyborohydride (317 mg, 1.50 mmol). The reaction mixture was maintained at 0° C. for 5 minutes, whereupon it was stirred at 25° C. for 15 minutes. After removal of dichloromethane via concentration under reduced pressure, water (40 mL) was added and the mixture was extracted with ethyl acetate (2×30 mL). The combined organic layers were washed with saturated aqueous sodium chloride solution, filtered, dried, concentrated in vacuo, and purified using silica gel chromatography (Gradient: 0% to 20% methanol in dichloromethane) to afford C78 as a brown-yellow solid. Yield: 170 mg, 0.181 mmol, 73%. LCMS m/z 941.3 [M+H]⁺.

Step 4. Synthesis of N-{[(1r,4r)-4-{6-[6-(3-{1-[(3RS)-2,6-dioxopiperidin-3-yl]-3-methyl-2-oxo-2,3-dihydro-1H-benzimidazol-5-yl}propyl)-6,7-dihydro-5H-pyrrolo[3,4-b]pyridin-2-yl]-2H-indazol-2-yl}cyclohexyl]methyl}-2,3,5-trifluoro-4-hydroxybenzamide (2): A solution of hydrogen chloride in 1,4-dioxane (4.0 M; 1.0 mL, 4.0 mmol) was added to a solution of C78 (170 mg, 0.181 mmol) in dichloromethane (10 mL). After the reaction mixture had been stirred at 25° C. for 30 minutes, it was concentrated in vacuo; the residue was purified using reversed-phase HPLC (Column: Welch Xtimate C18, 30×250 mm, 10 μm; Mobile phase A: water containing 0.1% formic acid; Mobile phase B: acetonitrile; Gradient: 18% to 95% B; Flow rate: 50 mL/minute) to provide N-{[(1r,4r)-4-{6-[6-(3-{1-[(3RS)-2,6-dioxopiperidin-3-yl]-3-methyl-2-oxo-2,3-dihydro-1H-benzimidazol-5-yl}propyl)-6,7-dihydro-5H-pyrrolo[3,4-b]pyridin-2-yl]-2H-indazol-2-yl}cyclohexyl]methyl}-2,3,5-trifluoro-4-hydroxybenzamide (2) as a light-yellow solid. Yield: 41.3 mg, 50.3 μmol, 28%. LCMS m/z 821.2 [M+H]⁺. ¹H NMR (400 MHz, DMSO-d₆) δ 11.10 (s, 1H), 8.42 (s, 1H), 8.25 (s, 1H), 8.16-8.08 (m, 1H), 7.85 (d, half of AB quartet, J=8.0 Hz, 1H), 7.81-7.70 (m, 3H), 7.22 (ddd, J=11.6, 6.7, 2.2 Hz, 1H), 7.10 (br s, 1H), 6.98 (AB quartet, upfield doublet is broadened, J_{AB}=8.0 Hz, Δv_{AB}=38.8 Hz, 2H), 5.35 (dd, J=12.8, 5.4 Hz, 1H), 4.55-4.43 (m, 1H), 3.96 (s, 4H), 3.34 (s, 3H), 3.17 (t, J=6.3 Hz, 2H), 2.90 (ddd, J=17.1, 13.2, 5.3 Hz, 1H), 2.80-2.66 (m, 5H), 2.66-2.57 (m, 1H), 2.23-2.13 (m, 2H), 2.06-1.82 (m, 7H), 1.74-1.61 (m, 1H), 1.32-1.15 (m, 3H).

### Example 3

### N-{[4-(5-{2-[4-(3-{1-[(3RS)-2,6-Dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)bicyclo[2.2.2]octan-1-yl]methyl}-2,3,5-trifluoro-4-hydroxybenzamide, hydrochloride salt (3)

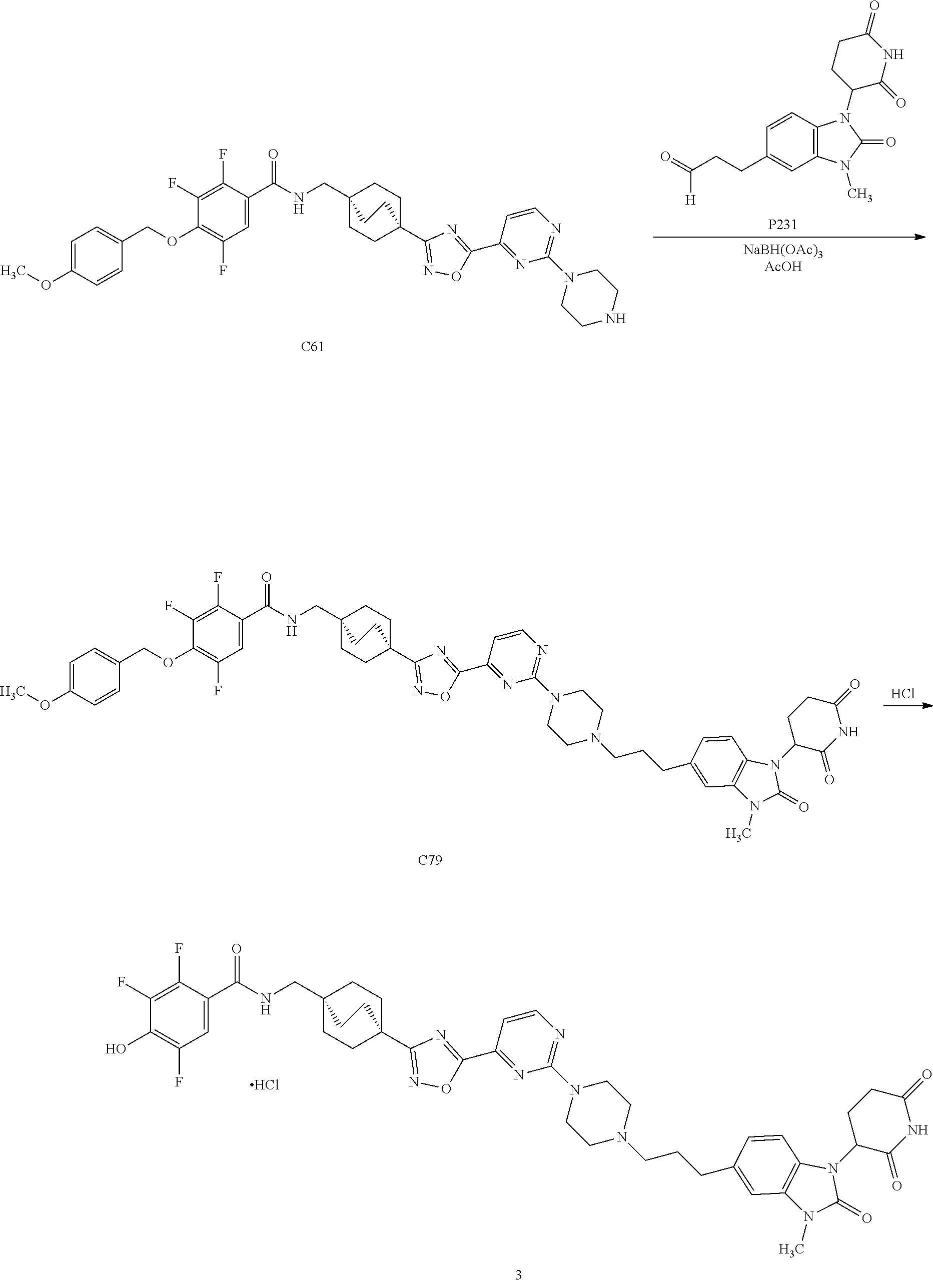

Step 1. Synthesis of N-[(4-{5-[2-(4-{3-[1-(2,6-dioxopiperidin-3-yl)-3-methyl-2-oxo-2,3-dihydro-1H-benzimidazol-5-yl]propyl}piperazin-1-yl)pyrimidin-4-yl]-1,2,4-oxadiazol-3-yl}bicyclo[2.2.2]octan-1-yl)methyl]-2,3,5-trifluoro-4-[(4-methoxyphenyl)methoxy]benzamide (C79): Acetic acid (1.30 mL, 22.7 mmol) was added drop-wise to a 0° C. solution of C61 (8.83 g, 13.3 mmol) in dichloromethane (133 mL). After addition of sodium triacetoxyborohydride (8.38 g, 39.5 mmol), stirring was continued at 0° C. for 5 minutes, whereupon P231 (92%, 4.75 g, 13.9 mmol) was added as a solid. The reaction mixture was stirred at 0° C. for an additional 40 minutes, then at room temperature for 30 minutes, before being partitioned between dichloromethane (300 mL) and saturated aqueous sodium bicarbonate solution (180 mL). The aqueous layer was extracted with dichloromethane (100 mL), and the combined organic layers were washed with saturated aqueous sodium chloride solution (150 mL), dried over sodium sulfate, filtered, and concentrated in vacuo. The resulting material was combined with the products of two similar reactions carried out using C61 (506 mg, 0.762 mmol; 98% purity, 500 mg, 0.738 mmol) and purified using silica gel chromatography (Gradient: 0% to 100% acetone in dichloromethane) to afford C79 as a pale-yellow solid. Combined yield: 12.8 g, 13.3 mmol, 90%. LCMS m/z 963.7 [M+H]⁺. ¹H NMR (400 MHz, chloroform-d) δ 8.50 (d, J=4.8 Hz, 1H), 8.32 (br s, 1H), 7.59 (ddd, J=11.7, 6.7, 2.3 Hz, 1H), 7.33 (d, J=8.6 Hz, 2H), 7.21 (d, J=4.8 Hz, 1H), 6.93-6.83 (m, 4H), 6.72 (d, J=8.0 Hz, 1H), 6.61-6.51 (m, 1H), 5.24 (s, 2H), 5.20 (dd, J=12.7, 5.4 Hz, 1H), 4.01-3.88 (m, 4H), 3.80 (s, 3H), 3.42 (s, 3H), 3.33-3.26 (m, 2H), 2.98-2.88 (m, 1H), 2.88-2.65 (m, 4H), 2.58-2.48 (m, 4H), 2.47-2.38 (m, 2H), 2.27-2.19 (m, 1H), 2.06-1.95 (m, 6H), 1.94-1.83 (m, 2H), 1.64-1.54 (m, 6H).

Step 2. Synthesis of N-{[4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)bicyclo[2.2.2]octan-1-yl]methyl}-2,3,5-trifluoro-4-hydroxybenzamide, hydrochloride salt (3): A solution of hydrogen chloride in 1,4-dioxane (4.0 M; 7.0 mL, 28 mmol) was added slowly, over approximately 3 minutes, to a 0° C. solution of C79 (1.71 g, 1.78 mmol) in dichloromethane (10 mL). After the resulting clumpy mixture had been stirred for 5 minutes at 0° C., additional dichloromethane (3 mL) was introduced. The reaction mixture was vigorously stirred for 1 hour at 0° C., whereupon it was removed from the cooling bath and stirred at room temperature for 2 hours. Solids were collected via rapid filtration using filter paper, suspended in acetonitrile (20 mL), and vigorously stirred at room temperature for 1 hour. A second rapid filtration through filter paper provided a filter cake; this was rinsed with acetonitrile (approximately 5 mL). The filter cake was suspended in acetone (approximately 25 mL) and vigorously stirred at 30° C. for 3 hours, then at room temperature overnight. Filtration, followed by rinsing of the filter cake with acetone (5 mL), afforded N-{[4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)bicyclo[2.2.2]octan-1-yl]methyl}-2,3,5-trifluoro-4-hydroxybenzamide, hydrochloride salt (3) as an off-white/pale-tan solid. Yield: 1.13 g, 1.28 mmol, 72%. LCMS m/z 843.7 [M+H]⁺. ¹H NMR (400 MHz, DMSO-d₆) δ 11.35 (br s, 1H), 11.11 (br s, 1H), 11.08 (s, 1H), 8.74 (d, J=4.9 Hz, 1H), 8.19 (br t, J=6 Hz, 1H), 7.40 (d, J=4.8 Hz, 1H), 7.27 (ddd, J=11.0, 6.2, 2.3 Hz, 1H), 7.10 (br s, 1H), 6.99 (AB quartet, upfield doublet is broadened, J_{AB}=8.1 Hz, Δv_{AB}=50.2 Hz, 2H), 5.35 (dd, J=12.7, 5.4 Hz, 1H), 4.77-4.69 (m, 2H), 3.65-3.56 (m, 2H), 3.56-3.45 (m, 2H), 3.34 (s, 3H), 3.15-2.99 (m, 6H), 2.98-2.84 (m, 1H), 2.78-2.56 (m, 4H), 2.16-2.04 (m, 2H), 2.04-1.95 (m, 1H), 1.94-1.83 (m, 6H), 1.59-1.47 (m, 6H).

### Example 4

### N-{[4-(2-{2-[4-(3-{1-[(3RS)-2,6-Dioxopiperidin-3-yl]-3-methyl-2-oxo-2,3-dihydro-1H-benzimidazol-5-yl}propyl)piperazin-1-yl]pyrimidin-4-yl}-1,3-thiazol-4-yl)bicyclo[2.2.2]octan-1-yl]methyl}-2,3,5-trifluoro-4-hydroxybenzamide, formate salt (4)

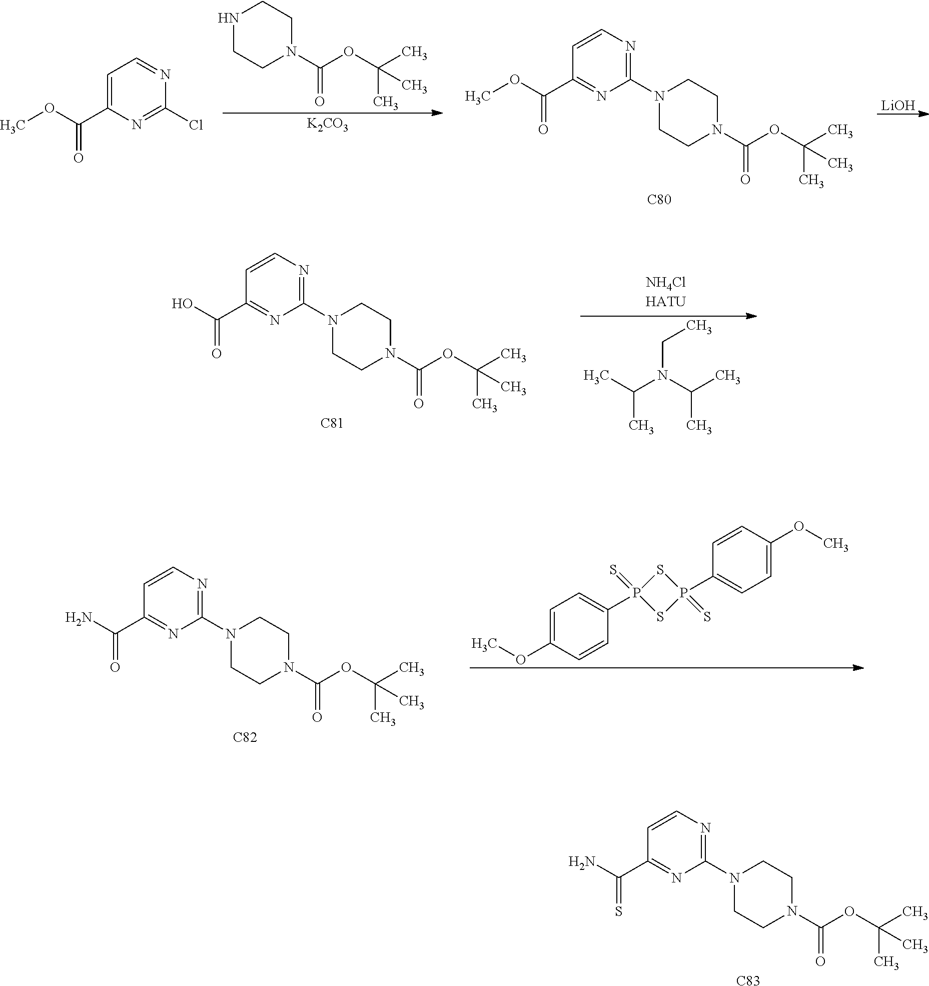

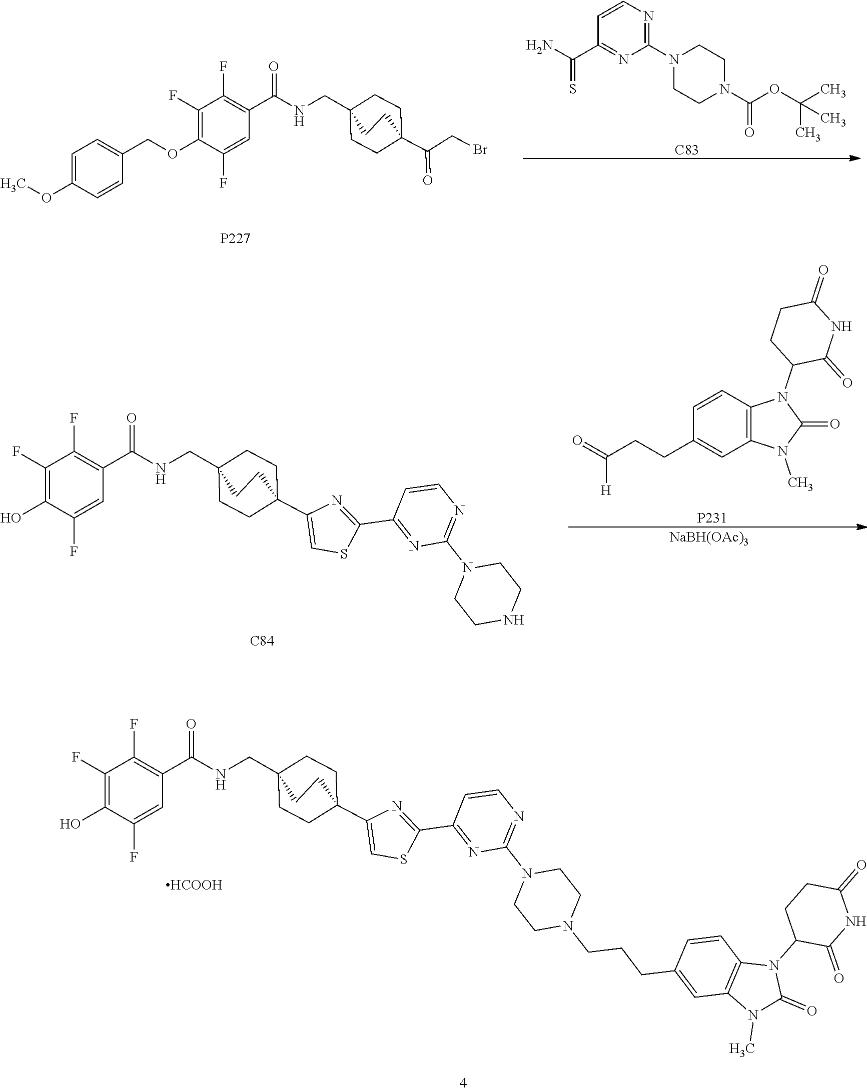

Step 1. Synthesis of methyl 2-[4-(tert-butoxycarbonyl)piperazin-1-yl]pyrimidine-4-carboxylate (C80): A mixture of methyl 2-chloropyrimidine-4-carboxylate (75.0 g, 435 mmol), tert-butyl piperazine-1-carboxylate (121 g, 650 mmol), and potassium carbonate (240 g, 1.74 mol) in acetonitrile (1.5 L) was stirred at 80° C. for 8 hours, whereupon LCMS analysis indicated conversion to C80: LCMS m/z 323.2 [M+H]⁺. The reaction mixture was filtered, and the filtrate was concentrated in vacuo to provide C80. A portion of this material (190 g) was used directly in the following reaction.

Step 2. Synthesis of 2-[4-(tert-butoxycarbonyl)piperazin-1-yl]pyrimidine-4-carboxylic acid (C81): To a solution of C80 (from the previous step; 90 g, 5206 mmol) in a mixture of tetrahydrofuran (1 L) and water (1 L) was added lithium hydroxide (66.9 g, 2.79 mol). After the reaction mixture had been stirred at 60° C. for 16 hours, the tetrahydrofuran was removed under reduced pressure. Water (500 mL) was added to the aqueous residue, and its pH was adjusted to 3; the resulting precipitate was collected by filtration, affording C81 as an off-white solid. Yield: 50 g, 160 mmol, 78% over 2 steps. LCMS m/z 309.2 [M+H]⁺. ¹H NMR (400 MHz, DMSO-d₆) δ 13.47 (br s, 1H), 8.59 (d, J=4.8 Hz, 1H), 7.11 (d, J=4.8 Hz, 1H), 3.80-3.73 (m, 4H), 3.44-3.37 (m, 4H), 1.42 (s, 9H).

Step 3. Synthesis of tert-butyl 4-(4-carbamoylpyrimidin-2-yl)piperazine-1-carboxylate (C82): To a solution of C81 (5.00 g, 16.2 mmol), ammonium chloride (1.04 g, 19.4 mmol), and O-(7-azabenzotriazol-1-yl)-N,N,N′N′-tetramethyluronium hexafluorophosphate (HATU; 7.39 g, 19.4 mmol) in N,N-dimethylformamide (40 mL) was added N,N-diisopropylethylamine (8.47 mL, 48.6 mmol), whereupon the reaction mixture was stirred at 25° C. for 6 hours. Water (80 mL) was added and the resulting mixture was extracted with ethyl acetate (2×100 mL); the combined organic layers were washed with saturated aqueous sodium chloride solution, dried over sodium sulfate, filtered, and concentrated in vacuo. Silica gel chromatography (Eluent: 20:1 dichloromethane/methanol) provided C82 as a solid. Yield: 4.60 g, 15.0 mmol, 93%. LCMS m/z 308.1 [M+H]⁺.

Step 4. Synthesis of tert-butyl 4-(4-carbamothioylpyrimidin-2-yl)piperazine-1-carboxylate (C83): 2,4-Bis(4-methoxyphenyl)-1,3,2,4-dithiadiphosphetane-2,4-dithione (3.95 g, 9.77 mmol) was added to a mixture of C82 (2.00 g, 6.51 mmol) in toluene (20 mL). The reaction mixture was stirred for 12 hours at 110° C., whereupon it was extracted with ethyl acetate (2×50 mL); the combined organic layers were washed with saturated aqueous sodium chloride solution, dried over sodium sulfate, filtered, and concentrated in vacuo. Purification via silica gel chromatography (Gradient: 0% to 100% ethyl acetate in petroleum ether) afforded C83 as a yellow solid, which contained impurities by ¹H NMR analysis. A portion of this material was used in the following reaction. Yield: 1.80 g, <5.57 mmol, <86%. LCMS m/z 324.2 [M+H]⁺. ¹H NMR (400 MHz, DMSO-d₆), product peaks only: δ 8.55 (d, J=4.9 Hz, 1H), 7.50 (d, J=4.9 Hz, 1H), 3.86-3.77 (m, 4H), 3.43-3.34 (m, 4H), 1.42 (s, 9H).

Step 5. Synthesis of 2,3,5-trifluoro-4-hydroxy-N-[(4-{2-[2-(piperazin-1-yl)pyrimidin-4-yl]-1,3-thiazol-4-yl}bicyclo[2.2.2]octan-1-yl)methyl]benzamide (C84): To a solution of P227 (200 mg, 0.361 mmol) in a mixture of 1,4-dioxane (2 mL) and toluene (2 mL) was added C83 (from the previous step; 117 mg, <0.362 mmol). The reaction mixture was stirred at 120° C. for 16 hours, whereupon it was extracted with ethyl acetate (3×40 mL). The combined organic layers were washed with saturated aqueous sodium chloride solution (50 mL) and concentrated in vacuo. Silica gel chromatography (Gradient: 0% to 30% methanol in dichloromethane) provided C84 as a yellow solid, which contained impurities. A portion of this material was progressed to the following step. Yield: 133 mg, <0.238 mmol. LCMS m/z 559.2 [M+H]⁺.

Step 6. Synthesis of N-{[4-(2-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,3-thiazol-4-yl)bicyclo[2.2.2]octan-1-yl]methyl}-2,3,5-trifluoro-4-hydroxybenzamide, formate salt (4): Sodium triacetoxyborohydride (133 mg, 0.627 mmol) was added to a solution of C84 (from the previous step; 70 mg, <0.13 mmol) and P231 (43.5 mg, 0.138 mmol) in dichloromethane (10 mL). After the reaction mixture had been stirred at 25° C. for 2 hours, it was concentrated in vacuo and purified, first by chromatography on silica gel (Gradient: 0% to 25% methanol in dichloromethane) and then via reversed-phase HPLC (Column: Welch Xtimate C18, 30×250 mm, 10 μm; Mobile phase A: water containing 0.1% formic acid; Mobile phase B: acetonitrile; Gradient: 25% to 95% B; Flow rate: 50 mL/minute), affording N-{[4-(2-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,3-thiazol-4-yl)bicyclo[2.2.2]octan-1-yl]methyl}-2,3,5-trifluoro-4-hydroxybenzamide, formate salt (4) as a white solid. Yield: 33.8 mg, 37.4 μmol, 17% over 3 steps. LCMS m/z 858.3 [M+H]⁺. ¹H NMR (400 MHz, DMSO-d₆), characteristic peaks: δ 11.09 (s, 1H), 8.49 (d, J=4.9 Hz, 1H), 8.15 (s, <1H, assumed to be formate signal), 8.05-7.99 (m, 1H), 7.46 (s, 1H), 7.26-7.20 (m, 1H), 7.21 (d, J=5.0 Hz, 1H), 7.07 (br s, 1H), 7.01 (d, half of AB quartet, J=8.0 Hz, 1H), 6.90 (dd, component of ABX system, J=8.1, 1.6 Hz, 1H), 5.34 (dd, J=12.7, 5.4 Hz, 1H), 3.83-3.75 (m, 4H), 3.33 (s, 3H, assumed; partially obscured by water peak), 3.06 (d, J=6.1 Hz, 2H), 2.96-2.83 (m, 1H), 2.77-2.56 (m, 4H), 2.40-2.33 (m, 2H), 2.05-1.95 (m, 1H), 1.90-1.74 (m, 8H), 1.57-1.47 (m, 6H).

### Example 5

### N-{[4-(4-{2-[4-(3-{1-[(3RS)-2,6-Dioxopiperidin-3-yl]-3-methyl-2-oxo-2,3-dihydro-1H-benzimidazol-5-yl}propyl)piperazin-1-yl]pyrimidin-4-yl}-1,3-thiazol-2-yl)bicyclo[2.2.2]octan-1-yl]methyl}-2,3,5-trifluoro-4-hydroxybenzamide (5)

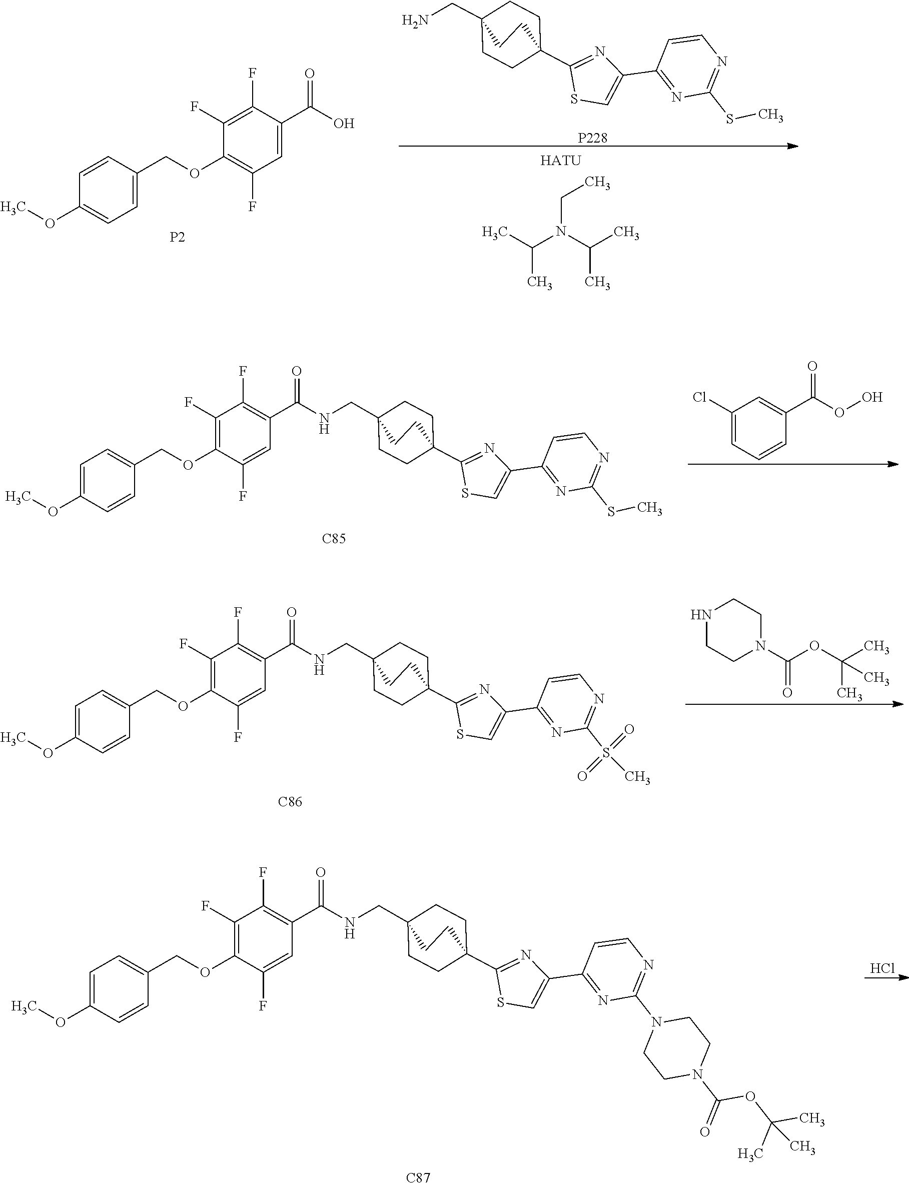

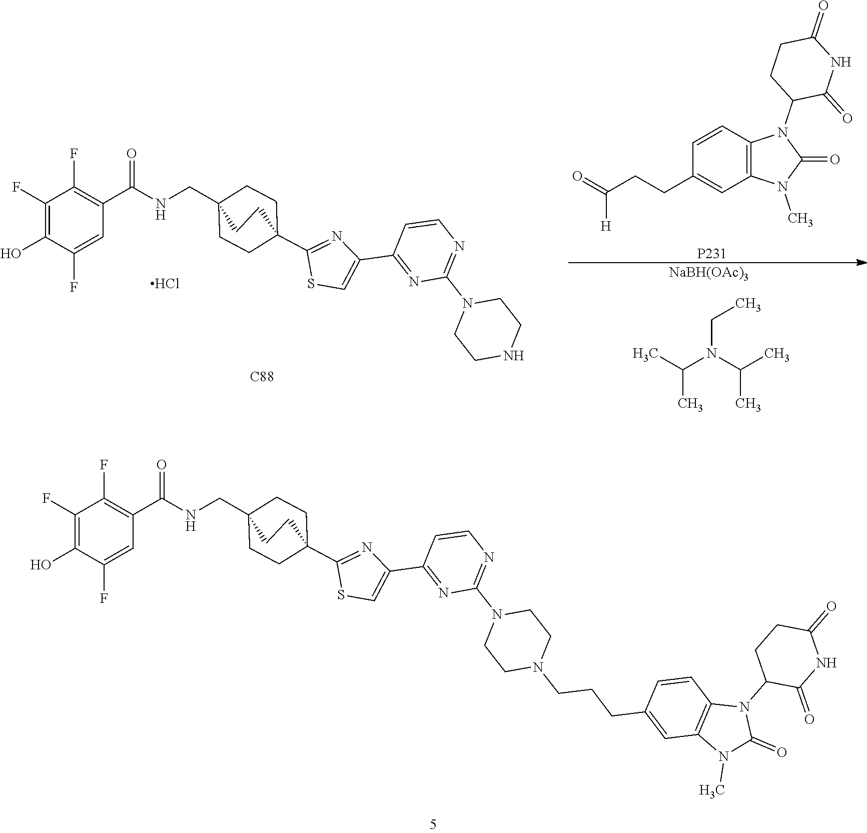

Step 1. Synthesis of 2,3,5-trifluoro-4-[(4-methoxyphenyl)methoxy]-N-[(4-{4-[2-(methylsulfanyl)pyrimidin-4-yl]-1,3-thiazol-2-yl}bicyclo[2.2.2]octan-1-yl)methyl]benzamide (C85): To a solution of P2 (200 mg, 0.641 mmol) and O-(7-azabenzotriazol-1-yl)-N,N,N′N′-tetramethyluronium hexafluorophosphate (HATU; 292 mg, 0.768 mmol) in dichloromethane (20 mL) was added N,N-diisopropylethylamine (248 mg, 1.92 mmol). After the mixture had been stirred at 25° C. for 2 minutes, P228 (222 mg, 0.641 mmol) was added, and the reaction mixture was stirred at 25° C. for 1 hour. Water (50 mL) was then added, and the resulting mixture was extracted with ethyl acetate (2×50 mL); the combined organic layers were washed with saturated aqueous sodium chloride solution, dried over sodium sulfate, filtered, and concentrated in vacuo. Silica gel chromatography (Gradient: 40% to 80% ethyl acetate in petroleum ether) afforded C85 as a solid. Yield: 286 mg, 0.446 mmol, 70%. LCMS m/z 641.3 [M+H]⁺. ¹H NMR (400 MHz, DMSO-d₆) δ 8.70 (d, J=5.1 Hz, 1H), 8.46 (s, 1H), 8.37 (br t, J=6.3 Hz, 1H), 7.68 (d, J=5.1 Hz, 1H), 7.38-7.30 (m, 1H), 7.36 (d, J=8.6 Hz, 2H), 6.94 (d, J=8.7 Hz, 2H), 5.22 (s, 2H), 3.75 (s, 3H), 3.07 (d, J=6.3 Hz, 2H), 2.57 (s, 3H), 1.98-1.90 (m, 6H), 1.60-1.51 (m, 6H).

Step 2. Synthesis of 2,3,5-trifluoro-N-[(4-{4-[2-(methanesulfonyl)pyrimidin-4-yl]-1,3-thiazol-2-yl}bicyclo[2.2.2]octan-1-yl)methyl]-4-[(4-methoxyphenyl)methoxy]benzamide (C86): 3-Chloroperoxybenzoic acid (308 mg, 1.78 mmol) was added to a solution of C85 (286 mg, 0.446 mmol) in dichloromethane (10 mL), whereupon the reaction mixture was stirred at 25° C. for 16 hours. It was then diluted with water (50 mL) and extracted with dichloromethane (2×50 mL); the combined organic layers were washed with saturated aqueous sodium chloride solution, dried over sodium sulfate, filtered, concentrated in vacuo, and purified via silica gel chromatography (Gradient: 40% to 80% ethyl acetate in petroleum ether), affording C86 as a yellow solid. Yield: 256 mg, 0.381 mmol, 85%. LCMS m/z 673.2 [M+H]⁺. ¹H NMR (400 MHz, DMSO-d₆) δ 9.11 (d, J=5.2 Hz, 1H), 8.69 (s, 1H), 8.37 (br t, J=6.2 Hz, 1H), 8.22 (d, J=5.2 Hz, 1H), 7.38-7.31 (m, 1H), 7.36 (d, J=8.6 Hz, 2H), 6.94 (d, J=8.7 Hz, 2H), 5.22 (s, 2H), 3.75 (s, 3H), 3.48 (s, 3H), 3.08 (d, J=6.2 Hz, 2H), 2.01-1.92 (m, 6H), 1.61-1.52 (m, 6H).

Step 3. Synthesis of tert-butyl 4-(4-{2-[4-({2,3,5-trifluoro-4-[(4-methoxyphenyl)methoxy]benzamido}methyl)bicyclo[2.2.2]octan-1-yl]-1,3-thiazol-4-yl}pyrimidin-2-yl)piperazine-1-carboxylate (C87): A solution of C86 (256 mg, 0.381 mmol) and tert-butyl piperazine-1-carboxylate (283 mg, 1.52 mmol) in dimethyl sulfoxide (5 mL) was stirred at 85° C. for 3 days, whereupon the reaction mixture was diluted with ethyl acetate (30 mL) and washed with water (2×30 mL). The organic layer was dried over sodium sulfate, filtered, concentrated in vacuo, and purified using silica gel chromatography (Eluent: 20:1 dichloromethane/methanol) to provide C87 as a yellow oil, which contained impurities. Most of this material was progressed to the following step. Yield: 168 mg, <0.216 mmol, <57%. LCMS m/z 779.4 [M+H]⁺. ¹H NMR (400 MHz, DMSO-d₆), characteristic peaks: δ 8.47 (d, J=4.9 Hz, 1H), 8.36 (s, 1H), 7.38-7.30 (m, 1H), 7.36 (d, J=8.6 Hz, 2H), 5.22 (s, 2H), 3.83-3.76 (m, 4H), 3.45-3.38 (m, 4H), 3.07 (d, J=6.2 Hz, 2H), 1.98-1.87 (m, 6H), 1.61-1.49 (m, 6H).

Step 4. Synthesis of 2,3,5-trifluoro-4-hydroxy-N-[(4-{4-[2-(piperazin-1-yl)pyrimidin-4-yl]-1,3-thiazol-2-yl}bicyclo[2.2.2]octan-1-yl)methyl]benzamide, hydrochloride salt (C88): A solution of hydrogen chloride in 1,4-dioxane (4 M; 1.5 mL, 6 mmol) was added to a solution of C87 (160 mg, <0.205 mmol) in dichloromethane (6 mL). The reaction mixture was stirred at 25° C. for 2 hours, whereupon it was concentrated in vacuo to provide C88, which contained impurities. The bulk of this material was progressed to the following step. Yield: 98 mg, <0.165 mmol. LCMS m/z 559.2 [M+H]⁺. ¹H NMR (400 MHz, DMSO-d₆), characteristic product peaks: δ 8.52 (d, J=5.0 Hz, 1H), 8.41 (s, 1H), 8.23-8.16 (m, 1H), 7.29 (d, J=5.0 Hz, 1H), 7.28-7.20 (m, 1H), 4.07-4.01 (m, 4H), 3.08 (d, J=6.3 Hz, 2H), 1.98-1.88 (m, 6H), 1.61-1.51 (m, 6H).

Step 5. Synthesis of N-{[4-(4-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,3-thiazol-2-yl)bicyclo[2.2.2]octan-1-yl]methyl}-2,3,5-trifluoro-4-hydroxybenzamide (5): Sodium triacetoxyborohydride (134 mg, 0.632 mmol) was added to a solution of C88 (from the previous step; 77.9 mg, <0.131 mmol), N,N-diisopropylethylamine (16.4 mg, 0.127 mmol), and P231 (40.0 mg, 0.127 mmol) in dichloromethane (10 mL). After the reaction mixture had been stirred at 25° C. for 10 hours, it was concentrated in vacuo. Purification via silica gel chromatography (Gradient: 0% to 25% methanol in dichloromethane), followed by reversed-phase HPLC (Column: Welch Xtimate C18, 30×250 mm, 10 μm; Mobile phase A: water containing 0.1% formic acid; Mobile phase B: acetonitrile; Gradient: 38% to 95% B; Flow rate: 50 mL/minute), afforded N-{[4-(4-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,3-thiazol-2-yl)bicyclo[2.2.2]octan-1-yl]methyl}-2,3,5-trifluoro-4-hydroxybenzamide (5) as a white solid. Yield: 25 mg, 29 μmol, 10% over 3 steps. LCMS m/z 858.3 [M+H]⁺. ¹H NMR (400 MHz, DMSO-d₆) δ 11.08 (s, 1H), 8.44 (d, J=5.0 Hz, 1H), 8.32 (s, 1H), 8.03-7.95 (m, 1H), 7.25-7.17 (m, 1H), 7.18 (d, J=5.0 Hz, 1H), 7.07 (br s, 1H), 6.95 (AB quartet, upfield doublet is broadened, J_{AB}=8.1 Hz, Δv_{AB}=44.1 Hz, 2H), 5.34 (dd, J=12.7, 5.4 Hz, 1H), 3.87-3.75 (m, 4H), 3.33 (s, 3H, assumed; partially obscured by water peak), 3.07 (d, J=6.2 Hz, 2H), 2.96-2.83 (m, 1H), [2.76-2.57 (m) and 2.5-2.42 (m), total 8H, assumed; partially obscured by solvent peak], 2.40-2.33 (m, 2H), 2.05-1.96 (m, 1H), 1.96-1.88 (m, 6H), 1.86-1.75 (m, 2H), 1.60-1.50 (m, 6H).

### Example 6

### N-{[(1r,4r)-4-{6-[4-({4-[({2-[(3RS)-2,6-Dioxopiperidin-3-yl]-1-oxo-2,3-dihydro-1H-isoindol-4-yl}oxy)methyl]phenyl}methyl)piperazin-1-yl]-2H-indazol-2-yl}cyclohexyl]methyl}-3,5-difluoro-4-hydroxybenzamide (6)

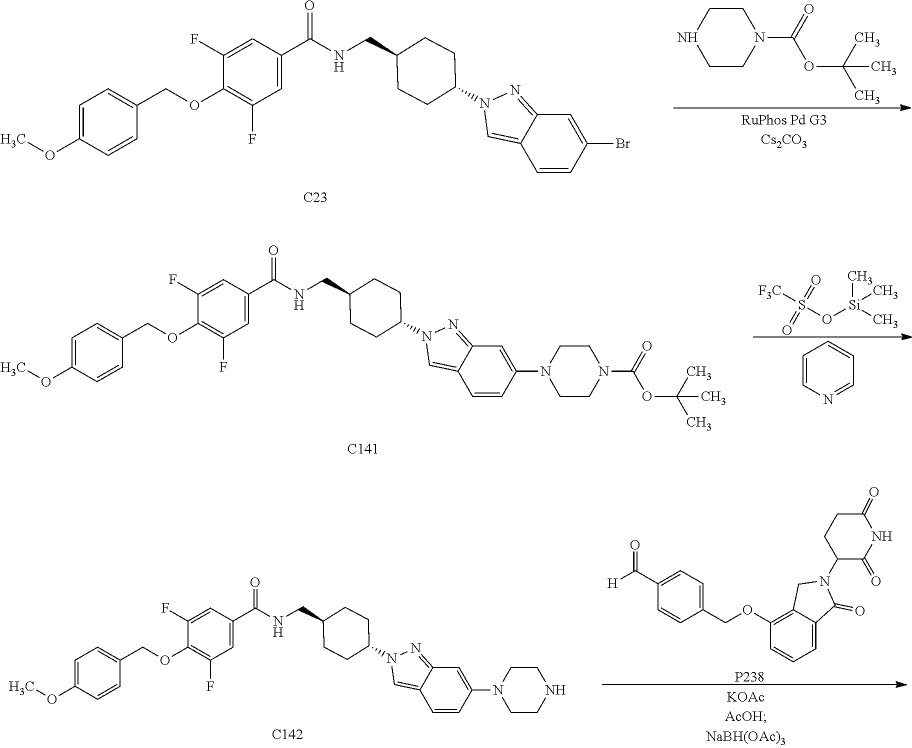

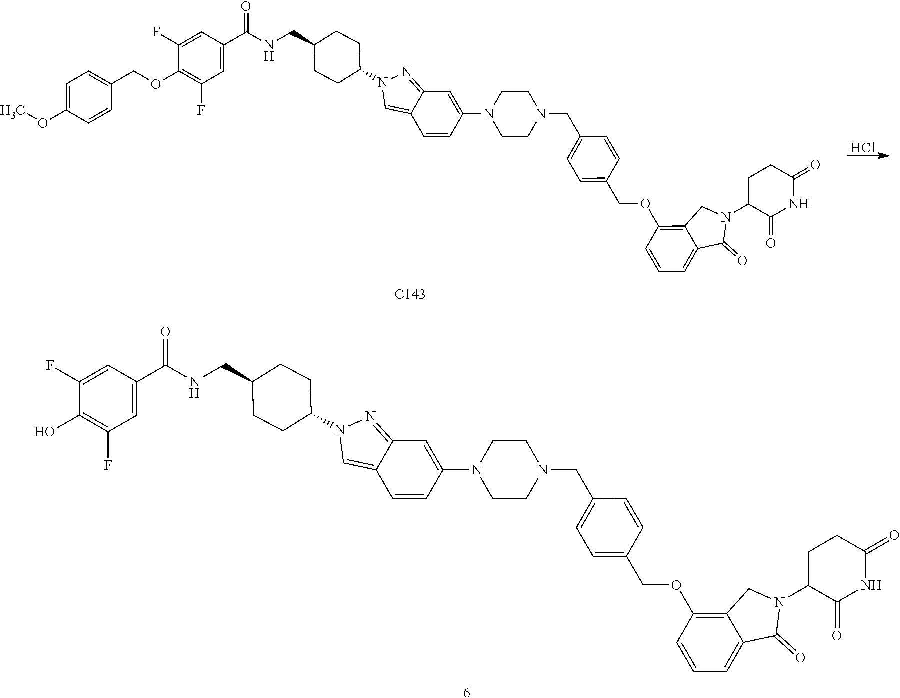 [SMILES: COc1ccc(COc2c(F)cc(C(=O)NC[C@H]3CC[C@H](n4cc5ccc(N6CCN(Cc7ccc(COc8cccc9c8CN(C8CCC(=O)NC8=O)C9=O)cc7)CC6)cc5n4)CC3)cc2F)cc1 ; Cl ; "C143" ; O=C1CCC(N2Cc3c(OCc4ccc(CN5CCN(c6ccc7cn([C@H]8CC[C@H](CNC(=O)c9cc(F)c(O)c(F)c9)CC8)nc7c6)CC5)cc4)cccc3C2=O)C(=O)N1 ; "6"]

Step 1. Synthesis of tert-butyl 4-{2-[(1r,4r)-4-({3,5-difluoro-4-[(4-methoxyphenyl)methoxy]benzamido}methyl)cyclohexyl]-2H-indazol-6-yl}piperazine-1-carboxylate (C141): To a solution of C23 (7.00 g, 12.0 mmol), tert-butyl piperazine-1-carboxylate (2.68 g, 14.4 mmol), and cesium carbonate (7.80 g, 23.9 mmol) in 1,4-dioxane (150 mL) was added (2-dicyclohexylphosphino-2′,6′-diisopropoxy-1,1′-biphenyl)[2-(2′-amino-1,1′-biphenyl)]palladium(II) methanesulfonate (RuPhos Pd G3; 1.50 g, 1.79 mmol), whereupon the reaction mixture was stirred at 100° C. for 16 hours and filtered. The filtrate was concentrated in vacuo; silica gel chromatography (Eluent: 5:3 petroleum ether/ethyl acetate) provided C141 as an off-yellow solid. Yield: 5.30 g, 7.68 mmol, 64%. LCMS m/z 690.3 [M+H]⁺. ¹H NMR (400 MHz, DMSO-d₆) δ 8.56 (br t, J=5.7 Hz, 1H), 8.18 (s, 1H), 7.65-7.56 (m, 2H), 7.51 (d, J=9.1 Hz, 1H), 7.34 (d, J=8.5 Hz, 2H), 6.96-6.87 (m, 3H), 6.84-6.80 (m, 1H), 5.17 (s, 2H), 4.42-4.29 (m, 1H), 3.74 (s, 3H), 3.54-3.41 (m, 4H), 3.17 (t, J=6.2 Hz, 2H), 3.11-2.99 (m, 4H), 2.16-2.05 (m, 2H), 1.96-1.77 (m, 4H), 1.73-1.58 (m, 1H), 1.42 (s, 9H), 1.28-1.10 (m, 2H).

Step 2. Synthesis of 3,5-difluoro-4-[(4-methoxyphenyl)methoxy]-N-({(1r,4r)-4-[6-(piperazin-1-yl)-2H-indazol-2-yl]cyclohexyl}methyl)benzamide (C142): To a 0° C. solution of C141 (5.60 g, 8.12 mmol) and pyridine (4.50 g, 56.9 mmol) in dichloromethane (50 mL) was added trimethylsilyl trifluoromethanesulfonate (12.6 g, 56.7 mmol). After the reaction mixture had been stirred at 25° C. for 16 hours, aqueous sodium bicarbonate solution (2 M; 300 mL) was added, and stirring was continued for 30 minutes. The resulting mixture was extracted with dichloromethane (2×300 mL), and the combined organic layers were washed with saturated aqueous sodium chloride solution, dried over sodium sulfate, filtered, and concentrated in vacuo. Silica gel chromatography (Eluent: 10:3 dichloromethane/methanol) afforded C142 as a solid. Yield: 4.53 g, 7.68 mmol, 95%. LCMS m/z 590.3 [M+H]⁺. ¹H NMR (400 MHz, DMSO-d₆) δ 8.59 (br t, J=5.8 Hz, 1H), 8.18 (s, 1H), 7.66-7.57 (m, 2H), 7.50 (d, J=9.1 Hz, 1H), 7.34 (d, J=8.6 Hz, 2H), 6.92 (d, J=8.6 Hz, 2H), 6.91-6.87 (m, 1H), 6.82-6.78 (m, 1H), 5.17 (s, 2H), 4.42-4.28 (m, 1H), 3.74 (s, 3H), 3.17 (t, J=6.3 Hz, 2H), 3.15-3.09 (m, 4H), 3.04-2.97 (m, 4H), 2.16-2.04 (m, 2H), 1.95-1.76 (m, 4H), 1.72-1.58 (m, 1H), 1.27-1.11 (m, 2H).

Step 3. Synthesis of N-({(1r,4r)-4-[6-(4-{[4-({[2-(2,6-dioxopiperidin-3-yl)-1-oxo-2,3-dihydro-1H-isoindol-4-yl]oxy}methyl)phenyl]methyl}piperazin-1-yl)-2H-indazol-2-yl]cyclohexyl}methyl)-3,5-difluoro-4-[(4-methoxyphenyl)methoxy]benzamide (C143): A solution of C142 (230 mg, 0.390 mmol), P238 (162 mg, 0.428 mmol), potassium acetate (191 mg, 1.95 mmol), and acetic acid (118 mg, 1.96 mmol) in dimethyl sulfoxide (10 mL) was stirred at 65° C. for 30 minutes, whereupon sodium triacetoxyborohydride (496 mg, 2.34 mmol) was added. After the reaction mixture had been stirred at 60° C. for an additional 16 hours, LCMS analysis indicated conversion to C143: LCMS m/z 952.4 [M+H]⁺. A similar reaction carried out using C142 (220 mg, 0.373 mmol) was added, and the combined reaction mixture was diluted with water (20 mL) and extracted with ethyl acetate (3×20 mL). The combined organic layers were washed with saturated aqueous sodium chloride solution (100 mL), dried over sodium sulfate, filtered, concentrated in vacuo, and purified using silica gel chromatography (Gradient: 0% to 10% methanol in dichloromethane) to afford C143 as an off-white solid. Combined yield: 320 mg, 0.336 mmol, 44%. ¹H NMR (400 MHz, DMSO-d₆), characteristic peaks: δ 8.14 (br s, 1H), 7.67 (s, 1H), 7.41-7.36 (m, 2H), 7.35-7.25 (m, 5H), 7.00 (d, J=8.0 Hz, 1H), 6.86 (br s, 1H), 6.78 (dd, J=9.2, 1.9 Hz, 1H), 6.75 (d, J=8.6 Hz, 1H), 6.18 (br t, J=6.0 Hz, 1H), 5.10 (dd, J=13.3, 5.1 Hz, 1H), 5.06 (s, 2H), 5.03 (s, 2H), 4.28 (AB quartet, J_{AB}=16.6 Hz, Δv_{AB}=51.1 Hz, 2H), 4.24-4.14 (m, 1H), 3.68 (s, 3H), 3.24 (t, J=6.5 Hz, 2H), 1.94-1.75 (m, 4H), 1.69-1.55 (m, 1H), 1.22-1.07 (m, 2H).

Step 4. Synthesis of N-{[(1r,4r)-4-{6-[4-({4-[({2-[(3RS)-2,6-dioxopiperidin-3-yl]-1-oxo-2,3-dihydro-1H-isoindol-4-yl}oxy)methyl]phenyl}methyl)piperazin-1-yl]-2H-indazol-2-yl}cyclohexyl]methyl}-3,5-difluoro-4-hydroxybenzamide (6): To a solution of C143 (310 mg, 0.326 mmol) in 1,4-dioxane (30 mL) was added a solution of hydrogen chloride in 1,4-dioxane (4 M; 3 mL, 12 mmol), whereupon the reaction mixture was stirred at 25° C. for 3 hours. It was then concentrated in vacuo and purified via chromatography on silica gel (Gradient: 0% to 20% methanol in dichloromethane), providing N-{[(1r,4r)-4-{6-[4-({4-[({2-[(3RS)-2,6-dioxopiperidin-3-yl]-1-oxo-2,3-dihydro-1H-isoindol-4-yl}oxy)methyl]phenyl}methyl)piperazin-1-yl]-2H-indazol-2-yl}cyclohexyl]methyl}-3,5-difluoro-4-hydroxybenzamide (6) as a solid. Yield: 78 mg, 93.8 μmol, 29%. LCMS m/z 832.3 [M+H]⁺. ¹H NMR (400 MHz, DMSO-d₆) δ 10.96 (s, 1H), 8.51 (br t, J=5.7 Hz, 1H), 8.16 (s, 1H), 7.66-7.55 (m, 2H), 7.53-7.43 (m, 4H), 7.43-7.29 (m, 4H), 6.88 (br d, J=9.2 Hz, 1H), 6.77 (br s, 1H), 5.24 (s, 2H), 5.11 (dd, J=13.3, 5.1 Hz, 1H), 4.39-4.28 (m, 1H), 4.34 (AB quartet, J_{AB}=17.5 Hz, Δv_{AB}=61.1 Hz, 2H), 3.67-3.47 (m, 2H), 3.22-3.00 (m, 6H), 2.97-2.84 (m, 1H), 2.67-2.37 (m, 5H, assumed; partially obscured by solvent peak), 2.15-2.04 (m, 2H), 2.03-1.94 (m, 1H), 1.94-1.77 (m, 4H), 1.72-1.58 (m, 1H), 1.27-1.11 (m, 3H).

### Example 7

### N-{[(1r,4r)-4-{6-[2-(2-{4-[3-(2,4-Dioxo-1,3-diazinan-1-yl)-4-methylbenzoyl]piperazin-1-yl}ethoxy)pyrimidin-5-yl]-2H-indazol-2-yl}cyclohexyl]methyl}-2,3,5-trifluoro-4-hydroxybenzamide, trifluoroacetate salt (7)

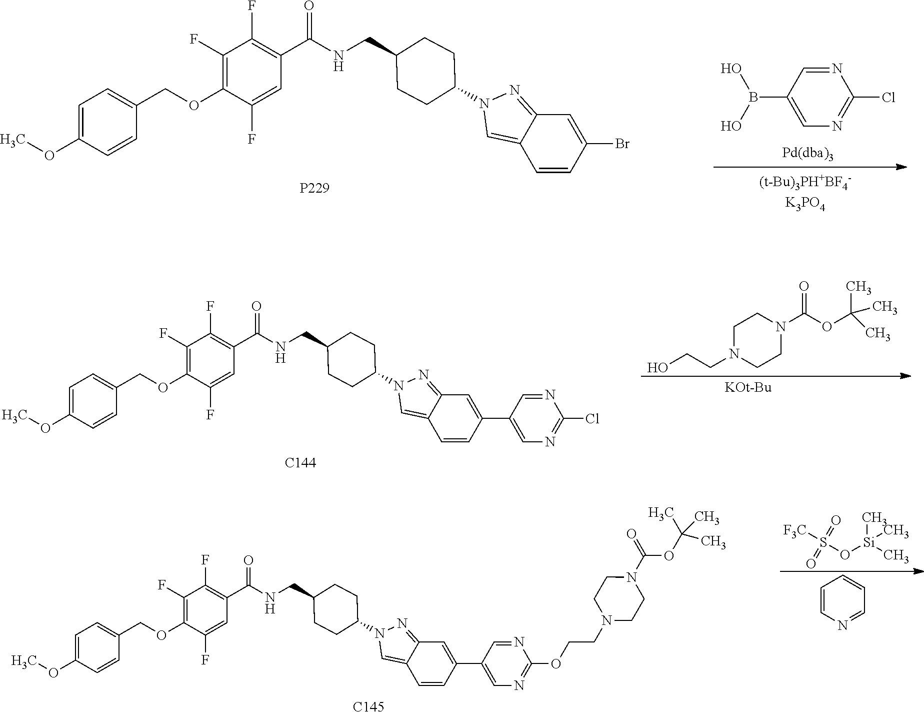

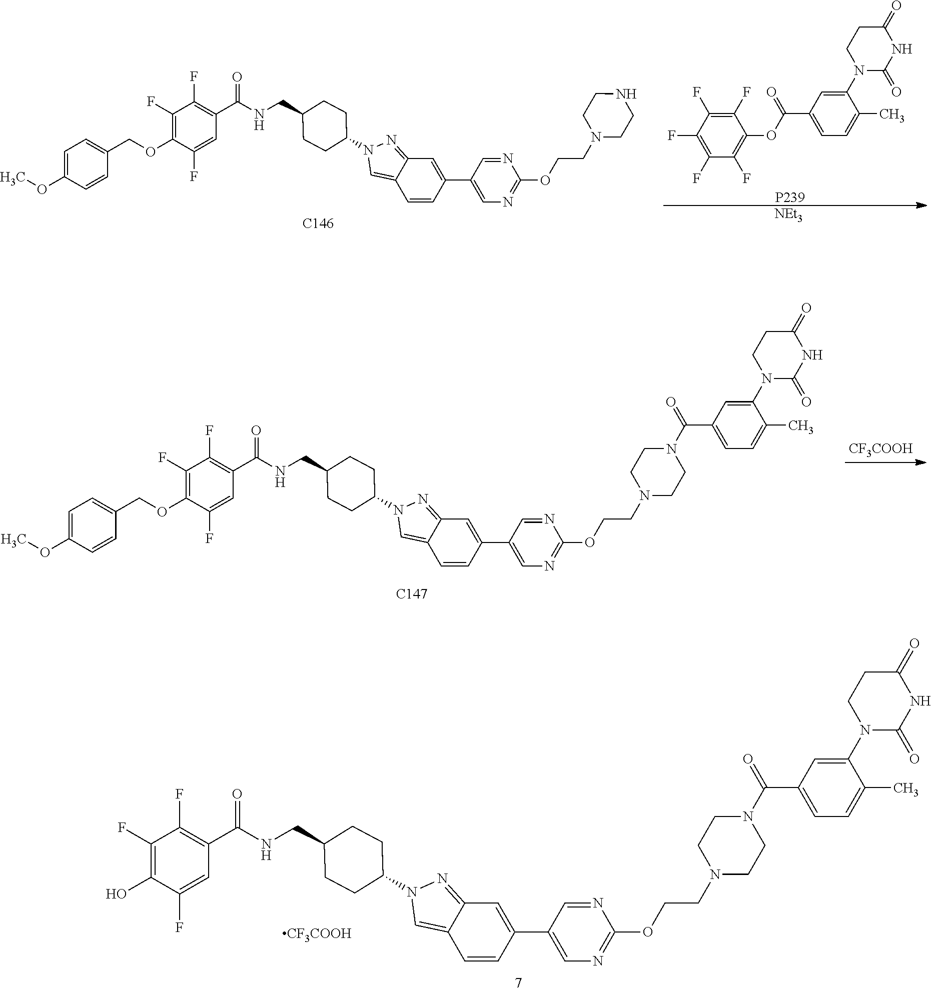

Step 1. Synthesis of N-({(1r,4r)-4-[6-(2-chloropyrimidin-5-yl)-2H-indazol-2-yl]cyclohexyl}methyl)-2,3,5-trifluoro-4-[(4-methoxyphenyl)methoxy]benzamide (C144): To a solution of P229 (3.00 g, 5.00 mmol) and (2-chloropyrimidin-5-yl)boronic acid (867 mg, 5.48 mmol) in a mixture of 1,4-dioxane (27 mL) and water (8.1 mL) were sequentially added tripotassium phosphate (97%, 2.18 g, 9.96 mmol), tris(dibenzylideneacetone)dipalladium(0) [Pd(dba)₃, 98%; 233 mg, 0.249 mmol), and tri-tert-butylphosphonium tetrafluoroborate (144 mg, 0.496 mmol). After the reaction mixture had been degassed with nitrogen for 4 minutes, it was stirred at 50° C. for 18 hours, whereupon it was filtered, while still warm, through diatomaceous earth. The filter pad was washed thoroughly with ethyl acetate, and the combined filtrates were concentrated under reduced pressure; the residue was partitioned between water and dichloromethane, and the aqueous layer was extracted three times with dichloromethane. The dichloromethane layers were combined, dried over sodium sulfate, filtered, concentrated in vacuo, and purified via silica gel chromatography (Gradient: 0% to 100% ethyl acetate in heptane) to provide C144 as an off-white solid. Yield: 2.10 g, 3.30 mmol, 66%. LCMS m/z 636.3 (chlorine isotope pattern observed) [M+H]⁺. ¹H NMR (400 MHz, chloroform-d) δ 8.89 (s, 2H), 8.03 (s, 1H), 7.92 (br s, 1H), 7.82 (br d, J=8.7 Hz, 1H), 7.60 (ddd, J=11.6, 6.8, 2.3 Hz, 1H), 7.34 (d, J=8.6 Hz, 2H), 7.29-7.25 (m, 1H, assumed; partially obscured by solvent peak), 6.88 (d, J=8.6 Hz, 2H), 6.72-6.61 (m, 1H), 5.25 (s, 2H), 4.56-4.43 (m, 1H), 3.80 (s, 3H), 3.43 (br t, J=6.2 Hz, 2H), 2.44-2.33 (m, 2H), 2.13-1.95 (m, 4H), 1.88-1.74 (m, 1H), 1.41-1.25 (m, 2H).

Step 2. Synthesis of tert-butyl 4-{2-[(5-{2-[(1r,4r)-4-({2,3,5-trifluoro-4-[(4-methoxyphenyl)methoxy]benzamido}methyl)cyclohexyl]-2H-indazol-6-yl}pyrimidin-2-yl)oxy]ethyl}piperazine-1-carboxylate (C145): A solution of potassium tert-butoxide in tetrahydrofuran (1 M; 3.08 mL, 3.08 mmol) was added to a solution of tert-butyl 4-(2-hydroxyethyl)piperazine-1-carboxylate (634 mg, 2.75 mmol) in tetrahydrofuran (10 mL). The reaction mixture was stirred at room temperature for 20 minutes, whereupon C144 (1.40 g, 2.20 mmol) was added and stirring was continued for 18 hours at room temperature. After the reaction mixture had been diluted with water and ethyl acetate, the mixture was filtered; the aqueous layer of the filtrate was extracted twice with ethyl acetate, and the combined organic layers were dried over magnesium sulfate, filtered, and concentrated in vacuo. Silica gel chromatography (Gradient: 0% to 10% methanol in dichloromethane, followed by a second column using a gradient of 0% to 100% ethyl acetate in heptane) afforded C145 as a white solid. Yield: 799 mg, 0.963 mmol, 44%. LCMS m/z 830.6 [M+H]⁺. ¹H NMR (400 MHz, chloroform-d) δ 8.77 (s, 2H), 7.99 (br s, 1H), 7.83 (br s, 1H), 7.76 (br d, J=8.7 Hz, 1H), 7.60 (ddd, J=11.7, 6.8, 2.3 Hz, 1H), 7.34 (d, J=8.6 Hz, 2H), 7.23 (dd, J=8.7, 1.5 Hz, 1H), 6.88 (d, J=8.7 Hz, 2H), 6.72-6.61 (m, 1H), 5.25 (s, 2H), 4.63-4.53 (m, 2H), 4.49-4.37 (m, 1H), 3.80 (s, 3H), 3.53-3.37 (m, 6H), 2.95-2.83 (m, 2H), 2.65-2.49 (m, 4H), 2.42-2.30 (m, 2H), 2.12-1.93 (m, 4H), 1.87-1.73 (m, 1H), 1.46 (s, 9H), 1.39-1.23 (m, 2H).

Step 3. Synthesis of 2,3,5-trifluoro-4-[(4-methoxyphenyl)methoxy]-N-{[(1r,4r)-4-(6-{2-[2-(piperazin-1-yl)ethoxy]pyrimidin-5-yl}-2H-indazol-2-yl)cyclohexyl]methyl}benzamide (C146): Trimethylsilyl trifluoromethanesulfonate (0.695 mL, 3.84 mmol) was added drop-wise to a 0° C. solution of C145 (797 mg, 0.960 mmol) in a mixture of pyridine (0.621 mL, 7.68 mmol) and dichloromethane (39 mL), whereupon the reaction mixture was allowed to stir in the ice bath overnight for 18 hours. After the reaction mixture had been cooled back to 0° C., it was again treated with pyridine (0.621 mL, 7.68 mmol) and trimethylsilyl trifluoromethanesulfonate (0.695 mL, 3.84 mmol), allowed to warm to room temperature, and stirred for 4 days. The reaction mixture was cooled to 0° C., and aqueous sodium bicarbonate solution (20 mL) was slowly added. The resulting mixture was stirred for 10 minutes, diluted with dichloromethane, and treated with saturated aqueous sodium chloride solution. The aqueous layer was extracted three times with ethyl acetate, and the combined ethyl acetate layers were dried over magnesium sulfate, filtered, and concentrated under reduced pressure. The dichloromethane layer was washed with saturated aqueous sodium chloride solution, dried over magnesium sulfate, added to the ethyl acetate concentrate, and concentrated in vacuo, providing C146 as an off-white solid. All of this material was progressed to the following step. LCMS m/z 730.4 [M+H]⁺. ¹H NMR (400 MHz, chloroform-d) δ 8.78 (s, 2H), 7.99 (s, 1H), 7.85-7.82 (m, 1H), 7.77 (br d, J=8.7 Hz, 1H), 7.60 (ddd, J=11.6, 6.7, 2.3 Hz, 1H), 7.34 (d, J=8.6 Hz, 2H), 7.23 (dd, J=8.7, 1.5 Hz, 1H), 6.88 (d, J=8.7 Hz, 2H), 6.71-6.61 (m, 1H), 5.25 (s, 2H), 4.56 (t, J=5.6 Hz, 2H), 4.49-4.37 (m, 1H), 3.80 (s, 3H), 3.43 (t, J=6.4 Hz, 2H), 3.23-3.13 (m, 4H), 2.91 (t, J=5.6 Hz, 2H), 2.88-2.78 (m, 4H), 2.42-2.30 (m, 2H), 2.13-1.93 (m, 4H), 1.86-1.73 (m, 1H), 1.39-1.23 (m, 2H).

Step 4. Synthesis of N-{[(1r,4r)-4-{6-[2-(2-{4-[3-(2,4-dioxo-1,3-diazinan-1-yl)-4-methylbenzoyl]piperazin-1-yl}ethoxy)pyrimidin-5-yl]-2H-indazol-2-yl}cyclohexyl]methyl}-2,3,5-trifluoro-4-[(4-methoxyphenyl)methoxy]benzamide (C147): To a solution of C146 (from the previous step: 50.960 mmol) in dichloromethane (4 mL) was added triethylamine (0.405 mL, 2.91 mmol), followed by P239 (397 mg, 0.958 mmol). After the reaction mixture had been stirred for 30 minutes at room temperature, it was treated with saturated aqueous sodium bicarbonate solution, whereupon the aqueous layer was extracted twice with dichloromethane. The combined organic layers were dried over magnesium sulfate, filtered, concentrated in vacuo, and purified using silica gel chromatography (Gradient: 0% to 10% methanol in dichloromethane), affording C147 as an off-white solid. Yield: 600 mg, 0.625 mmol, 65% over 2 steps. LCMS m/z 960.7 [M+H]⁺. ¹H NMR (400 MHz, chloroform-d), characteristic peaks: δ 8.77 (s, 2H), 7.99 (s, 1H), 7.86 (br s, 1H), 7.77 (d, J=8.7 Hz, 1H), 7.65-7.57 (m, 2H), 7.34 (d, J=8.6 Hz, 2H), 7.33-7.20 (m, 3H, assumed; partially obscured by solvent peak), 6.88 (d, J=8.7 Hz, 2H), 6.72-6.60 (m, 1H), 5.25 (s, 2H), 4.70-4.52 (m, 2H), 4.52-4.40 (m, 1H), 3.91-3.78 (m, 1H), 3.81 (s, 3H), 3.70-3.60 (m, 1H), 3.43 (t, J=6.3 Hz, 2H), 2.98-2.79 (m, 4H), 2.42-2.33 (m, 2H), 2.31 (s, 3H), 2.11-1.92 (m, 4H), 1.88-1.73 (m, 1H), 1.41-1.24 (m, 2H).

Step 5. Synthesis of N-{[(1r,4r)-4-{6-[2-(2-{4-[3-(2,4-dioxo-1,3-diazinan-1-yl)-4-methylbenzoyl]piperazin-1-yl}ethoxy)pyrimidin-5-yl]-2H-indazol-2-yl}cyclohexyl]methyl}-2,3,5-trifluoro-4-hydroxybenzamide, trifluoroacetate salt (7): To a 0° C. solution of C147 (600 mg, 0.625 mmol) in dichloromethane (4 mL) was added trifluoroacetic acid (0.720 mL, 9.35 mmol), whereupon the ice bath was removed and the reaction mixture was stirred for 30 minutes at room temperature. The reaction mixture was concentrated in vacuo, and the resulting syrup was dissolved in dichloromethane (1.5 mL), cooled to 0° C., and again treated with trifluoroacetic acid (1.0 mL, 13 mmol); the cooling bath was removed and the reaction mixture was stirred for 30 minutes at room temperature. After addition of dichloromethane (20 mL), the mixture was cooled to 0° C., and saturated aqueous sodium bicarbonate solution was slowly added until the aqueous phase reached pH 7. The organic layer and precipitated solids were treated with tetrahydrofuran and methanol until a solution was obtained, whereupon acetic acid (3 drops) was added, followed by silica gel. The resulting mixture was concentrated under reduced pressure, and the product-laden silica gel was used as a pre-column for silica gel chromatography (Gradient: 0% to 10% methanol in dichloromethane). The purified material was combined with the product from a similar reaction carried out using C147 (249 mg, 0.259 mmol) and subjected to a second purification via silica gel chromatography (Gradient: 0% to 7.5% methanol in dichloromethane); the resulting white solid was subjected to a final purification using reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 20% to 40% B over 8.5 minutes, then 40% to 95% B over 0.5 minutes; Flow rate: 25 mL/minute) to provide N-{[(1r,4r)-4-{6-[2-(2-{4-[3-(2,4-dioxo-1,3-diazinan-1-yl)-4-methylbenzoyl]piperazin-1-yl}ethoxy)pyrimidin-5-yl]-2H-indazol-2-yl}cyclohexyl]methyl}-2,3,5-trifluoro-4-hydroxybenzamide, trifluoroacetate salt (7). Combined yield: 231 mg, 0.242 mmol, 27%. LCMS m/z 840.7 [M+H]⁺. ¹H NMR (400 MHz, methanol-d₄) δ 8.88 (s, 2H), 8.33 (br s, 1H), 7.87-7.81 (m, 2H), 7.42 (d, half of AB quartet, J=7.8 Hz, 1H), 7.38-7.31 (m, 3H), 7.28 (ddd, J=11.1, 6.3, 2.3 Hz, 1H), 4.64 (t, J=5.5 Hz, 2H), 4.57-4.45 (m, 1H), 3.91-3.49 (m, 6H), 3.38-3.3 (m, 2H, assumed; obscured by solvent peak), 2.94 (t, J=5.5 Hz, 2H), 2.90-2.58 (m, 6H), 2.32 (s, 3H), 2.32-2.25 (m, 2H), 2.12-1.98 (m, 4H), 1.89-1.75 (m, 1H), 1.42-1.27 (m, 2H).

### Example 8

### N-{[(1r,4r)-4-(6-{5-[4-(2-{1-[(3RS)-2,6-Dioxopiperidin-3-yl]-3-methyl-2-oxo-2,3-dihydro-1H-benzimidazol-5-yl}ethyl)piperazin-1-yl]pyrazin-2-yl}-2H-indazol-2-yl)cyclohexyl]methyl}-2,3,5-trifluoro-4-hydroxybenzamide, hydrochloride salt (8)

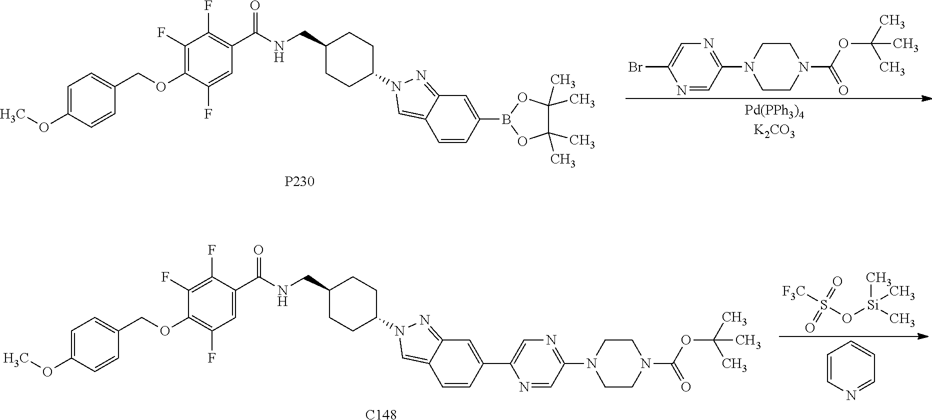

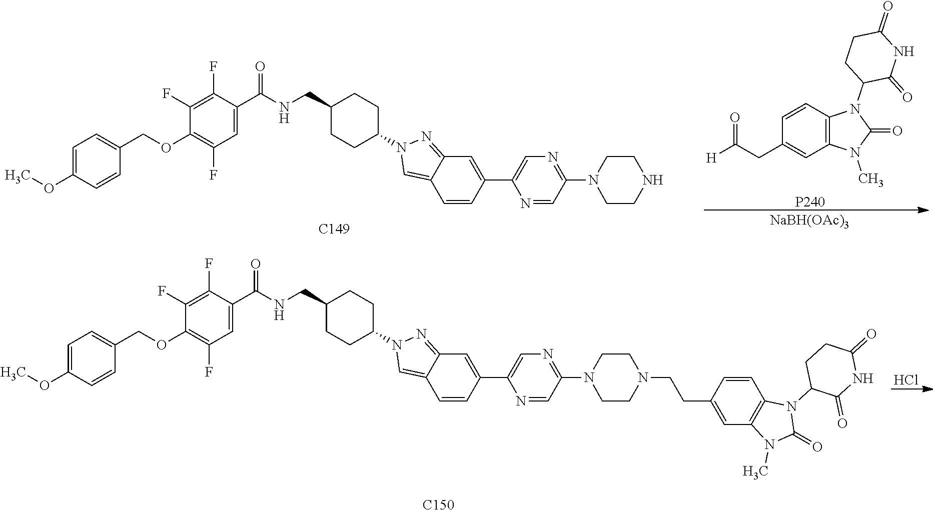

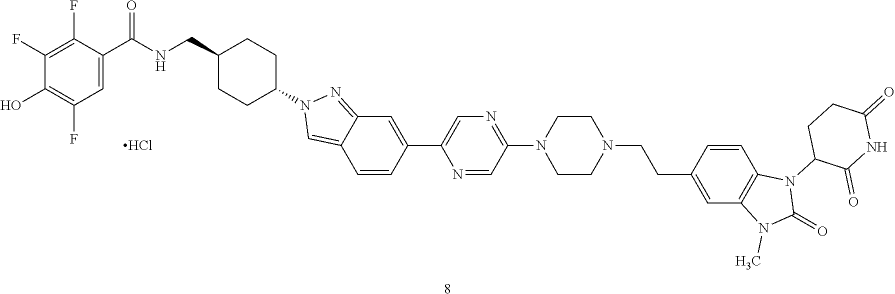 [SMILES: Cn1c(=O)n(C2CCC(=O)NC2=O)c2ccc(CCN3CCN(c4cnc(-c5ccc6cn([C@H]7CC[C@H](CNC(=O)c8cc(F)c(O)c(F)c8F)CC7)nc6c5)cn4)CC3)cc21 ; Cl ; "8"]

Step 1. Synthesis of tert-butyl 4-(5-{2-[(1r,4r)-4-({2,3,5-trifluoro-4-[(4-methoxyphenyl)methoxy]benzamido}methyl)cyclohexyl]-2H-indazol-6-yl}pyrazin-2-yl)piperazine-1-carboxylate (C148): A solution of P230 (2.90 g, 4.46 mmol), tert-butyl 4-(5-bromopyrazin-2-yl)piperazine-1-carboxylate (1.64 g, 4.78 mmol), potassium carbonate (1.80 g, 13.0 mmol), and tetrakis(triphenylphosphine)palladium(0) (519 mg, 0.449 mmol) in a mixture of 1,4-dioxane (40 mL) and water (10 mL) was degassed for 5 minutes, whereupon the reaction mixture was heated at 90° C. for 6 hours. After gradual removal of solvents in vacuo at 40° C., the residue was diluted with dichloromethane and filtered through diatomaceous earth; the filter pad was rinsed well with dichloromethane, and the organic layer of the combined filtrates was concentrated under reduced pressure, to a volume of approximately 10 mL. Acetonitrile (approximately 740 mL) was slowly added, and the resulting suspension was stirred for 30 minutes. Filtration, followed by rinsing of the filter cake with cold acetonitrile, afforded C148 as a white solid. Yield: 2.12 g, 2.70 mmol, 60%. LCMS m/z 786.6 [M+H]⁺. ¹H NMR (400 MHz, chloroform-d) δ 8.62 (s, 1H), 8.22 (s, 1H), 8.16 (s, 1H), 7.93 (s, 1H), 7.76-7.67 (m, 2H), 7.60 (ddd, J=11.7, 6.8, 2.3 Hz, 1H), 7.34 (d, J=8.3 Hz, 2H), 6.88 (d, J=8.5 Hz, 2H), 6.71-6.61 (m, 1H), 5.24 (s, 2H), 4.47-4.36 (m, 1H), 3.80 (s, 3H), 3.68-3.54 (m, 8H), 3.42 (t, J=6.4 Hz, 2H), 2.41-2.30 (m, 2H), 2.11-1.92 (m, 4H), 1.86-1.72 (m, 1H), 1.50 (s, 9H), 1.38-1.22 (m, 2H).

Step 2. Synthesis of 2,3,5-trifluoro-4-[(4-methoxyphenyl)methoxy]-N-{[(1r,4r)-4-{6-[5-(piperazin-1-yl)pyrazin-2-yl]-2H-indazol-2-yl}cyclohexyl]methyl}benzamide (C149): Trimethylsilyl trifluoromethanesulfonate (1.89 mL, 10.4 mmol) was added drop-wise to a −20° C. (methanol/ice cooling bath) solution of C148 (2.05 g, 2.61 mmol) and pyridine (1.69 mL, 20.9 mmol) in dichloromethane (84 mL). The reaction mixture was stirred in this methanol/ice bath overnight, whereupon the temperature of the bath had risen to 12° C. The reaction mixture was cooled to 0° C.; aqueous sodium bicarbonate solution (30 mL) was slowly added, and the mixture was stirred for 10 minutes. The aqueous layer was then adjusted to pH 10 and extracted three times with dichloromethane. The combined organic layers were washed sequentially with saturated sodium bicarbonate solution and saturated aqueous sodium chloride solution, dried over magnesium sulfate, filtered, and concentrated in vacuo. The resulting material was stirred for 30 minutes with heptane (approximately 10 mL) and an extremely small amount of dichloromethane; filtration provided C149 as a tan solid. Yield: 1.78 g, 2.60 mmol, quantitative. LCMS m/z 686.5 [M+H]⁺. ¹H NMR (400 MHz, DMSO-d₆) δ 8.74 (d, J=1.5 Hz, 1H), 8.50 (br t, J=6 Hz, 1H), 8.41-8.35 (m, 2H), 8.15 (br s, 1H), 7.74 (d, half of AB quartet, J=8.8 Hz, 1H), 7.69 (dd, component of ABX system, J=8.8, 1.4 Hz, 1H), 7.40-7.32 (m, 3H), 6.94 (d, J=8.7 Hz, 2H), 5.22 (s, 2H), 4.53-4.41 (m, 1H), 3.75 (s, 3H), 3.57-3.50 (m, 4H), 3.18 (t, J=6.3 Hz, 2H), 2.85-2.77 (m, 4H), 2.22-2.12 (m, 2H), 1.99-1.84 (m, 4H), 1.73-1.60 (m, 1H), 1.30-1.15 (m, 2H).

Step 3. Synthesis of N-{[(1r,4r)-4-{6-[5-(4-{2-[1-(2,6-dioxopiperidin-3-yl)-3-methyl-2-oxo-2,3-dihydro-1H-benzimidazol-5-yl]ethyl}piperazin-1-yl)pyrazin-2-yl]-2H-indazol-2-yl}cyclohexyl]methyl}-2,3,5-trifluoro-4-[(4-methoxyphenyl)methoxy]benzamide (C150): A mixture of C149 (1.15 g, 1.68 mmol) and P240 (75%, 741 mg, 1.84 mmol) in tetrahydrofuran (27 mL) was stirred at room temperature for 30 minutes, treated with sodium triacetoxyborohydride (1.42 g, 6.70 mmol), and heated to 50° C. for 1.5 hours, followed by stirring at room temperature overnight. The reaction mixture was then diluted with dichloromethane (220 mL) and washed with saturated aqueous sodium bicarbonate solution (100 mL). After the aqueous layer had been extracted with dichloromethane (100 mL), the combined organic layers were washed with saturated aqueous sodium chloride solution (50 mL), dried over sodium sulfate, filtered, and concentrated in vacuo to afford a pale-yellow solid (1.57 g). A portion of this material (820 mg) was purified via silica gel chromatography (Gradient: 0% to 20% propan-2-ol in dichloromethane), loaded as a mixture in dichloromethane containing a minimal quantity of methanol; this provided C150 as an off-white solid. Yield: 438 mg, 0.451 mmol, (51%, adjusted to account for purification on only part of the crude product). LCMS m/z 971.7 [M+H]⁺. ¹H NMR (400 MHz, DMSO-d₆) δ 11.08 (s, 1H), 8.76 (br s, 1H), 8.50 (br t, J=6 Hz, 1H), 8.44-8.37 (m, 2H), 8.17 (br s, 1H), 7.74 (br d, half of AB quartet, J=8.8 Hz, 1H), 7.69 (dd, component of ABX system, J=8.7, 1.4 Hz, 1H), 7.40-7.32 (m, 3H), 7.11 (br s, 1H), 7.02 (d, half of AB quartet, J=8.0 Hz, 1H), 6.97-6.90 (m, 3H), 5.34 (dd, J=12.7, 5.4 Hz, 1H), 5.22 (s, 2H), 4.54-4.41 (m, 1H), 3.75 (s, 3H), 3.68-3.58 (m, 4H), 3.33 (s, 3H), 3.18 (t, J=6.3 Hz, 2H), 2.97-2.54 (m, 11H), 2.22-2.12 (m, 2H), 2.05-1.84 (m, 5H), 1.72-1.60 (m, 1H), 1.31-1.15 (m, 2H).

Step 4. Synthesis of N-{[(1r,4r)-4-(6-{5-[4-(2-{1-[(3RS)-2,6-dioxopiperidin-3-yl]-3-methyl-2-oxo-2,3-dihydro-1H-benzimidazol-5-yl}ethyl)piperazin-1-yl]pyrazin-2-yl}-2H-indazol-2-yl)cyclohexyl]methyl}-2,3,5-trifluoro-4-hydroxybenzamide, hydrochloride salt (8): A solution of hydrogen chloride in 1,4-dioxane (4.0 M; 6.37 mL, 25.5 mmol) was slowly added, over 5 minutes, to a 0° C. solution of C150 (750 mg, 0.772 mmol) in dichloromethane (16 mL). After the reaction mixture had been stirred for 15 minutes at 0° C. and 1 hour at room temperature, it was filtered; the collected solids were isolated via filtration, suspended in acetonitrile (8.5 mL), and vigorously stirred at room temperature for 30 minutes. Filtration afforded a solid, which was suspended in propan-2-ol (8.5 mL), vigorously stirred at room temperature for 30 minutes, and collected via filtration to provide N-{[(1r,4r)-4-(6-{5-[4-(2-{1-[(3RS)-2,6-dioxopiperidin-3-yl]-3-methyl-2-oxo-2,3-dihydro-1H-benzimidazol-5-yl}ethyl)piperazin-1-yl]pyrazin-2-yl}-2H-indazol-2-yl)cyclohexyl]methyl}-2,3,5-trifluoro-4-hydroxybenzamide, hydrochloride salt (8) as a yellow solid. Yield: 506 mg, 0.570 mmol, 74%. LCMS m/z 851.7 [M+H]⁺. ¹H NMR (400 MHz, DMSO-d₆), characteristic peaks: δ 11.09 (s, 1H), 8.85 (d, J=1.4 Hz, 1H), 8.55 (br s, 1H), 8.42 (br s, 1H), 8.38-8.31 (m, 1H), 8.22 (br s, 1H), 7.77 (br d, half of AB quartet, J=8.8 Hz, 1H), 7.72 (dd, component of ABX system, J=8.8, 1.4 Hz, 1H), 7.30 (ddd, J=11.1, 6.3, 2.3 Hz, 1H), 7.14 (br s, 1H), 7.10 (d, half of AB quartet, J=8.1 Hz, 1H), 6.97 (dd, component of ABX system, J=8.2, 1.5 Hz, 1H), 5.37 (dd, J=12.8, 5.4 Hz, 1H), 3.72-3.63 (m, 2H), 3.53-3.34 (m, 4H, assumed; partially obscured by water peak), 3.25-3.08 (m, 6H), 2.97-2.84 (m, 1H), 2.78-2.57 (m, 2H), 2.23-2.12 (m, 2H), 2.05-1.85 (m, 5H), 1.75-1.61 (m, 1H), 1.32-1.16 (m, 2H).

### Example 9

### N-{[(1r,4r)-4-{6-[4-(2-{1-[(3RS)-2,6-Dioxopiperidin-3-yl]-3-methyl-2-oxo-2,3-dihydro-1H-benzimidazol-5-yl}ethyl)piperazin-1-yl]-2H-indazol-2-yl}cyclohexyl]methyl}-2,3,5-trifluoro-4-hydroxybenzamide, hydrochloride salt (9)

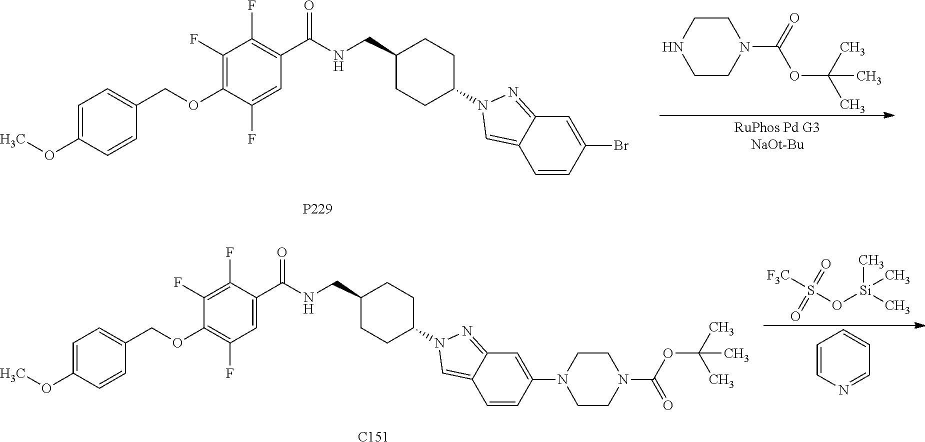

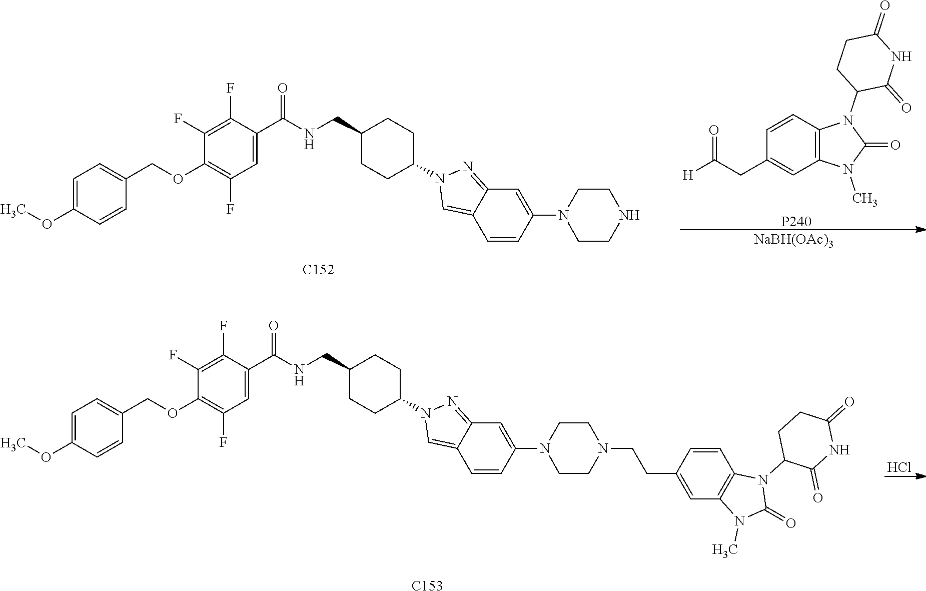

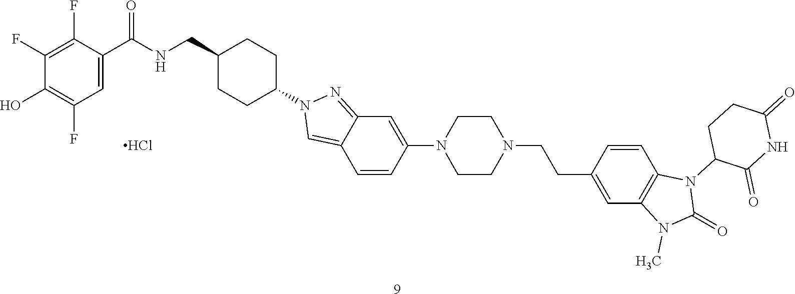 [SMILES: Cn1c(=O)n(C2CCC(=O)NC2=O)c2ccc(CCN3CCN(c4ccc5cn([C@H]6CC[C@H](CNC(=O)c7cc(F)c(O)c(F)c7F)CC6)nc5c4)CC3)cc21 ; Cl ; "9"]

Step 1. Synthesis of tert-butyl 4-{2-[(1r,4r)-4-({2,3,5-trifluoro-4-[(4-methoxyphenyl)methoxy]benzamido}methyl)cyclohexyl]-2H-indazol-6-yl}piperazine-1-carboxylate (C151): A mixture of P229 (1.91 g, 3.17 mmol), (2-dicyclohexylphosphino-2′,6′-diisopropoxy-1,1′-biphenyl)[2-(2′-amino-1,1′-biphenyl)]palladium(II) methanesulfonate (RuPhos Pd G3; 266 mg, 0.318 mmol), tert-butyl piperazine-1-carboxylate (680 mg, 3.65 mmol), and sodium tert-butoxide (915 mg, 9.52 mmol) in toluene (25 mL) was sparged with nitrogen for 5 minutes at room temperature, whereupon the reaction mixture was stirred at 90° C. for 5 hours. After the reaction mixture had cooled to room temperature, it was gently concentrated in vacuo (150 mbar gradually ramped down to 30 mbar, 25° C.) to remove most of the toluene. The residue was partitioned between ethyl acetate (250 mL) and water (50 mL), and the aqueous layer was extracted with ethyl acetate (2×50 mL). The combined organic layers were washed with saturated aqueous sodium chloride solution (50 mL), dried over sodium sulfate, filtered, and concentrated in vacuo; silica gel chromatography (Gradient: 30% to 100% acetonitrile in dichloromethane, followed by 100% acetonitrile) afforded C151 as an off-white/pale-gray solid. Yield: 1.65 g, 2.33 mmol, 74%. LCMS m/z 708.4 [M+H]⁺. ¹H NMR (400 MHz, chloroform-d) δ 7.80 (s, 1H), 7.59 (ddd, J=11.7, 6.7, 2.3 Hz, 1H), 7.52 (d, J=9.1 Hz, 1H), 7.34 (d, J=8.2 Hz, 2H), 6.99 (s, 1H), 6.94-6.84 (m, 3H), 6.69-6.60 (m, 1H), 5.24 (s, 2H), 4.38-4.26 (m, 1H), 3.80 (s, 3H), 3.66-3.56 (m, 4H), 3.41 (t, J=6.4 Hz, 2H), 3.18-3.08 (m, 4H), 2.36-2.25 (m, 2H), 2.08-1.87 (m, 4H), 1.83-1.69 (m, 1H), 1.49 (s, 9H), 1.35-1.20 (m, 2H).

Step 2. Synthesis of 2,3,5-trifluoro-4-[(4-methoxyphenyl)methoxy]-N-({(1r,4r)-4-[6-(piperazin-1-yl)-2H-indazol-2-yl]cyclohexyl}methyl)benzamide (C152): Trimethylsilyl trifluoromethanesulfonate (1.65 mL, 9.12 mmol) was added drop-wise to a −15° C. (methanol/ice bath slurry) solution of C151 (1.61 g, 2.27 mmol) and pyridine (1.45 mL, 17.9 mmol) in dichloromethane (75 mL). The reaction mixture was stirred for 13 hours, at which time the internal temperature was 16° C.; ice was added to the cooling bath until the internal temperature reached 2° C., whereupon saturated aqueous sodium bicarbonate solution (45 mL) was slowly added, followed by saturated aqueous sodium carbonate solution (10 mL). After the resulting mixture had been stirred for 10 minutes, the aqueous layer was extracted with dichloromethane (2×75 mL), and the combined organic layers were washed sequentially with saturated aqueous sodium carbonate solution (25 mL) and saturated aqueous sodium chloride solution (50 mL), dried over sodium sulfate, filtered, and concentrated in vacuo. The residue was treated with minimal dichloromethane; heptane was added to precipitate the product. The mixture was stirred at room temperature for 30 minutes, followed by filtration to afford C152 as a granular, off-white solid. Yield: 1.37 g, 2.25 mmol, 99%. LCMS m/z 608.4 [M+H]⁺. ¹H NMR (400 MHz, chloroform-d) δ 7.79 (s, 1H), 7.64-7.55 (m, 1H), 7.51 (d, J=9.1 Hz, 1H), 7.34 (d, J=8.2 Hz, 2H), 6.99 (s, 1H), 6.94-6.83 (m, 3H), 6.70-6.59 (m, 1H), 5.24 (s, 2H), 4.37-4.25 (m, 1H), 3.80 (s, 3H), 3.40 (t, J=6.5 Hz, 2H), 3.21-3.12 (m, 4H), 3.12-3.04 (m, 4H), 2.36-2.25 (m, 2H), 2.08-1.68 (m, 5H, assumed; partially obscured by water peak), 1.35-1.20 (m, 2H).

Step 3. Synthesis of N-({(1r,4r)-4-[6-(4-{2-[1-(2,6-dioxopiperidin-3-yl)-3-methyl-2-oxo-2,3-dihydro-1H-benzimidazol-5-yl]ethyl}piperazin-1-yl)-2H-indazol-2-yl]cyclohexyl}methyl)-2,3,5-trifluoro-4-[(4-methoxyphenyl)methoxy]benzamide (C153): A mixture of C152 (2.73 g, 4.49 mmol) and P240 (85%, 1.81 g, 5.11 mmol) in tetrahydrofuran (60 mL) was stirred at room temperature for 1 hour, whereupon sodium triacetoxyborohydride (3.00 g, 14.2 mmol) was added. The reaction mixture was placed in a preheated oil bath (50° C.) and stirred at 50° C. for an hour and 40 minutes, followed by stirring at room temperature for 30 minutes. It was then poured into dichloromethane (600 mL), and washed with saturated aqueous sodium bicarbonate solution (100 mL). The aqueous layer was extracted with dichloromethane (100 mL), and the combined dichloromethane layers were washed with saturated aqueous sodium chloride solution (100 mL), dried over sodium sulfate, filtered through diatomaceous earth, and concentrated in vacuo to provide C153 as an off-white/pale-tan solid (4.22 g). This material was progressed directly to the following step. LCMS m/z 893.5 [M+H]⁺.

Step 4. Synthesis of N-{[(1r,4r)-4-{6-[4-(2-{1-[(3RS)-2,6-dioxopiperidin-3-yl]-3-methyl-2-oxo-2,3-dihydro-1H-benzimidazol-5-yl}ethyl)piperazin-1-yl]-2H-indazol-2-yl}cyclohexyl]methyl}-2,3,5-trifluoro-4-hydroxybenzamide, hydrochloride salt (9): A solution of hydrogen chloride in 1,4-dioxane (4.0 M; 30.0 mL, 120 mmol) was slowly added, over 5 minutes, to a 0° C. solution of C153 (from the previous step; 4.22 g, 54.49 mmol) in dichloromethane (50 mL). The reaction mixture was stirred for 2 hours, while the cooling bath warmed; the solids were then collected via filtration and rinsed with 1,4-dioxane (5 mL). The filter cake was suspended in acetonitrile (8 mL), vigorously stirred at room temperature for 1 hour, and again collected by filtration. This filter cake was suspended in propan-2-ol (8 mL), vigorously stirred at room temperature for 1 hour, and filtered again, affording N-{[(1r,4r)-4-{6-[4-(2-{1-[(3RS)-2,6-dioxopiperidin-3-yl]-3-methyl-2-oxo-2,3-dihydro-1H-benzimidazol-5-yl}ethyl)piperazin-1-yl]-2H-indazol-2-yl}cyclohexyl]methyl}-2,3,5-trifluoro-4-hydroxybenzamide, hydrochloride salt (9) as an off-white/pale-tan solid. Yield: 2.45 g, 3.03 mmol, 67% over 2 steps. LCMS m/z 773.5 [M+H]⁺. ¹H NMR (400 MHz, DMSO-d₆) δ 11.38 (v br s, 1H), 11.19-10.98 (m, 2H), 8.39-8.32 (m, 1H), 8.31 (s, 1H), 7.60 (d, J=9.0 Hz, 1H), 7.30 (ddd, J=11.1, 6.3, 2.3 Hz, 1H), 7.15 (s, 1H), 7.11 (d, half of AB quartet, J=8.1 Hz, 1H), 7.04-6.90 (m, 3H), 5.38 (dd, J=12.8, 5.4 Hz, 1H), 4.48-4.34 (m, 1H), 3.91-3.80 (m, 2H), 3.71-3.60 (m, 2H), 3.45-3.36 (m, 2H), 3.35 (s, 3H), 3.29-3.10 (m, 8H), 2.98-2.85 (m, 1H), 2.78-2.57 (m, 2H), 2.20-2.08 (m, 2H), 2.06-1.96 (m, 1H), 1.96-1.80 (m, 4H), 1.73-1.58 (m, 1H), 1.30-1.13 (m, 2H).

### Example 10

### N-{[(1r,4r)-4-{6-[5-(4-{3-[3-(2,4-Dioxo-1,3-diazinan-1-yl)pyrazolo[1,5-a]pyridin-6-yl]propyl}piperazin-1-yl)pyrazin-2-yl]-2H-indazol-2-yl}cyclohexyl]methyl}-2,3,5-trifluoro-4-hydroxybenzamide, hydrochloride salt (10)

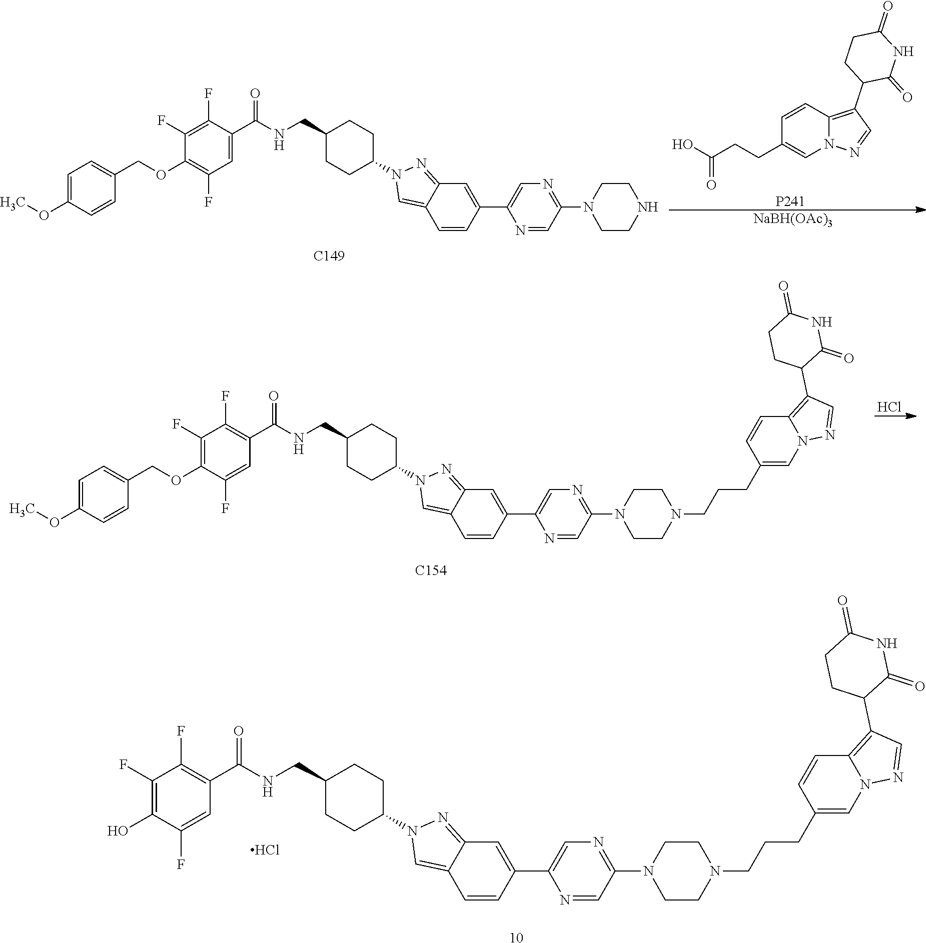

Step 1. Synthesis of N-{[(1r,4r)-4-{6-[5-(4-{3-[3-(2,4-dioxo-1,3-diazinan-1-yl)pyrazolo[1,5-a]pyridin-6-yl]propyl}piperazin-1-yl)pyrazin-2-yl]-2H-indazol-2-yl}cyclohexyl]methyl}-2,3,5-trifluoro-4-[(4-methoxyphenyl)methoxy]benzamide (C154): A solution of C149 (450 mg, 0.656 mmol) and P241 (80%, 245 mg, 0.685 mmol) in tetrahydrofuran (5 mL) was stirred for 1 hour at room temperature, whereupon sodium triacetoxyborohydride (278 mg, 1.31 mmol) was added in one portion and the reaction mixture was heated at 50° C. for 1 hour. Heating was then stopped, and the reaction mixture was allowed to cool to room temperature and stir for 18 hours before being partitioned between dichloromethane (40 mL) and saturated aqueous sodium bicarbonate solution (15 mL). After the mixture had been stirred for 20 minutes, the aqueous layer was extracted twice with dichloromethane; the combined organic layers were washed with saturated aqueous sodium chloride solution, dried over magnesium sulfate, and treated with silica gel. This mixture was concentrated to dryness and subjected to chromatography on silica gel (Gradient: 0% to 12% methanol in dichloromethane), providing C154 as a tan solid. Yield: 245 mg, 0.256 mmol, 39%. LCMS m/z 956.6 [M+H]⁺. ¹H NMR (400 MHz, chloroform-d) δ 8.61 (d, J=1.4 Hz, 1H), 8.25 (s, 1H), 8.22 (d, J=1.5 Hz, 1H), 8.17-8.14 (m, 1H), 7.93 (br s, 1H), 7.89 (s, 1H), 7.75-7.68 (m, 2H), 7.60 (ddd, J=11.7, 6.7, 2.3 Hz, 1H), 7.49 (br s, 1H), 7.37 (d, half of AB quartet J=9.3 Hz, 1H), 7.34 (d, J=8.6 Hz, 2H), 7.10 (dd, component of ABX system, J=9.1, 1.5 Hz, 1H), 6.88 (d, J=8.6 Hz, 2H), 6.71-6.60 (m, 1H), 5.25 (s, 2H), 4.47-4.36 (m, 1H), 3.89 (t, J=6.7 Hz, 2H), 3.80 (s, 3H), 3.75-3.60 (m, 4H), 3.42 (t, J=6.3 Hz, 2H), 2.90 (t, J=6.7 Hz, 2H), 2.72 (t, J=7.4 Hz, 2H), 2.66-2.53 (m, 4H), 2.51-2.41 (m, 2H), 2.41-2.31 (m, 2H), 2.11-1.85 (m, 6H), 1.85-1.72 (m, 1H), 1.38-1.23 (m, 2H).

Step 2. Synthesis of N-{[(1r,4r)-4-{6-[5-(4-{3-[3-(2,4-dioxo-1,3-diazinan-1-yl)pyrazolo[1,5-a]pyridin-6-yl]propyl}piperazin-1-yl)pyrazin-2-yl]-2H-indazol-2-yl}cyclohexyl]methyl}-2,3,5-trifluoro-4-hydroxybenzamide, hydrochloride salt (10): To a 0° C. mixture of C154 (245 mg, 0.256 mmol) in dichloromethane (3.0 mL) was slowly added a solution of hydrogen chloride in 1,4-dioxane (4.0 M; 2.0 mL, 8.0 mmol). The cooling bath was removed and the reaction mixture was stirred at room temperature for 90 minutes, whereupon it was filtered, and the filter cake was rinsed with dichloromethane (1 mL). The collected solids were slurried in acetonitrile (2 mL) with rapid stirring for 1 hour; filtration afforded a solid, which was isolated via filtration and rinsed with acetonitrile (1 mL). This material was slurried in propan-2-ol (2 mL) for 2 hours with rapid stirring, then filtered, affording N-{[(1r,4r)-4-{6-[5-(4-{3-[3-(2,4-dioxo-1,3-diazinan-1-yl)pyrazolo[1,5-a]pyridin-6-yl]propyl}piperazin-1-yl)pyrazin-2-yl]-2H-indazol-2-yl}cyclohexyl]methyl}-2,3,5-trifluoro-4-hydroxybenzamide, hydrochloride salt (10) as a yellow solid. Yield: 95.4 mg, 0.109 mmol, 43%. LCMS m/z 836.7 [M+H]⁺. ¹H NMR (400 MHz, DMSO-d₆) δ 11.06 (br s, 1H), 10.43 (s, 1H), 8.83 (s, 1H), 8.57 (s, 1H), 8.52 (s, 1H), 8.42 (s, 1H), 8.39-8.31 (m, 1H), 8.21 (s, 1H), 7.98 (s, 1H), 7.74 (AB quartet, J_{AB}=8.8 Hz, Δv_{AB}=17.9 Hz, 2H), 7.57 (d, J=9.1 Hz, 1H), 7.35-7.26 (m, 1H), 7.21 (d, J=9.1 Hz, 1H), 4.59-4.40 (m, 3H), 3.80-3.72 (m, 2H), 3.66-3.57 (m, 2H), 3.53-3.40 (m, 2H), 3.25-3.03 (m, 6H), 2.82-2.67 (m, 4H), 2.24-2.08 (m, 4H), 2.01-1.83 (m, 4H), 1.75-1.60 (m, 1H), 1.33-1.14 (m, 2H).

### Example 11

### N-{[(1r,4r)-4-{6-[2-(4-{8-[3-(2,4-Dioxo-1,3-diazinan-1-yl)-4-methylbenzoyl]-1-oxa-8-azaspiro[4.5]decan-3-yl}piperazin-1-yl)pyrimidin-5-yl]-2H-indazol-2-yl}cyclohexyl]methyl}-2,3,5-trifluoro-4-hydroxybenzamide (11)

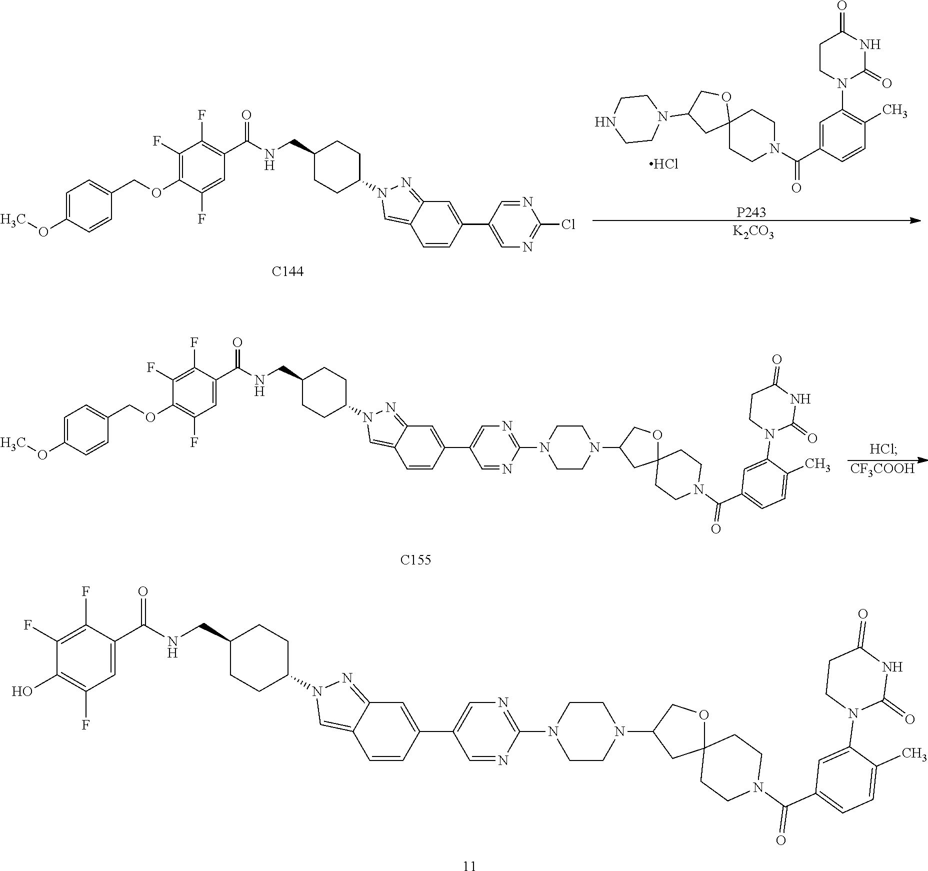

Step 1. Synthesis of N-{[(1r,4r)-4-{6-[2-(4-{8-[3-(2,4-dioxo-1,3-diazinan-1-yl)-4-methylbenzoyl]-1-oxa-8-azaspiro[4.5]decan-3-yl}piperazin-1-yl)pyrimidin-5-yl]-2H-indazol-2-yl}cyclohexyl]methyl}-2,3,5-trifluoro-4-[(4-methoxyphenyl)methoxy]benzamide (C155): Potassium carbonate (289 mg, 2.09 mmol) was added to a solution of P243 (206.0 mg, 0.419 mmol) and C144 (293 mg, 0.461 mmol) in N,N-dimethylformamide (4.0 mL). The reaction mixture was gradually heated to 85° C., then allowed to stir overnight at 85° C. After it had cooled, the reaction mixture was added to a mixture of cold water (40 mL) and saturated aqueous sodium chloride solution (10 mL). The resulting suspension was extracted with ethyl acetate (4×60 mL) and the combined organic layers were dried over sodium sulfate, filtered, and concentrated in vacuo to provide C155 as a solid (500 mg). This material was progressed directly to the following step. LCMS m/z 1055.7 [M+H]⁺.

Step 2. Synthesis of N-{[(1r,4r)-4-{6-[2-(4-{8-[3-(2,4-dioxo-1,3-diazinan-1-yl)-4-methylbenzoyl]-1-oxa-8-azaspiro[4.5]decan-3-yl}piperazin-1-yl)pyrimidin-5-yl]-2H-indazol-2-yl}cyclohexyl]methyl}-2,3,5-trifluoro-4-hydroxybenzamide (11): A solution of hydrogen chloride in 1,4-dioxane (4.0 M; 4.74 mL, 19.0 mmol) was slowly added to a solution of C155 (from the previous step; 500 mg, 50.419 mmol) in dichloromethane (8.0 mL), resulting in an immediate precipitate. The liquid was decanted away, and the solid was dissolved in 1,1,1,3,3,3-hexafluoropropan-2-ol (2.0 mL). Trifluoroacetic acid (0.10 mL, 1.3 mmol) was added, and the reaction mixture was stirred for two hours, whereupon it was concentrated in vacuo. The residue was subjected to supercritical fluid chromatography {Column: Regis Technologies, (S,S)-Whelk-O 1, 30×250 mm, 5 μm; Mobile phase: 7:3 carbon dioxide/[1:1:0.2 acetonitrile/propan-2-ol/(7 M ammonia in methanol)]; Flow rate: 80 mL/minute; Back pressure: 100 bar}, followed by silica gel chromatography (Gradient: 0% to 30% methanol in dichloromethane), affording N-{[(1r,4r)-4-{6-[2-(4-{8-[3-(2,4-dioxo-1,3-diazinan-1-yl)-4-methylbenzoyl]-1-oxa-8-azaspiro[4.5]decan-3-yl}piperazin-1-yl)pyrimidin-5-yl]-2H-indazol-2-yl}cyclohexyl]methyl}-2,3,5-trifluoro-4-hydroxybenzamide (11) as a solid. Yield: 65 mg, 70 μmol, 17% over 2 steps. LCMS m/z 935.7 [M+H]⁺. ¹H NMR (400 MHz, DMSO-d₆), characteristic peaks: δ 10.37 (s, 1H), 8.76 (s, 2H), 8.40 (s, 1H), 8.34-8.27 (m, 1H), 7.84 (br s, 1H), 7.75 (d, J=8.7 Hz, 1H), 7.38-7.23 (m, 5H), 4.54-4.41 (m, 1H), 3.97 (t, J=7.6 Hz, 1H), 3.63 (t, J=8.0 Hz, 1H), 3.58-3.48 (m, 1H), 3.22-3.14 (m, 2H), 3.05-2.93 (m, 1H), 2.84-2.63 (m, 2H), 2.45-2.36 (m, 2H), 2.21 (s, 3H), 2.21-2.11 (m, 2H), 2.05 (dd, J=12.2, 7.6 Hz, 1H), 1.99-1.84 (m, 4H), 1.76-1.47 (m, 6H), 1.31-1.15 (m, 2H).

### Example 12

### N-{[4-(7-{2-[4-(3-{1-[(3RS)-2,6-Dioxopiperidin-3-yl]-3-methyl-2-oxo-2,3-dihydro-1H-benzimidazol-5-yl}propyl)piperazin-1-yl]pyrimidin-5-yl}imidazo[1,2-a]pyridin-2-yl)bicyclo[2.2.2]octan-1-yl]methyl}-2,3,5-trifluoro-4-hydroxybenzamide, trifluoroacetate salt (12)

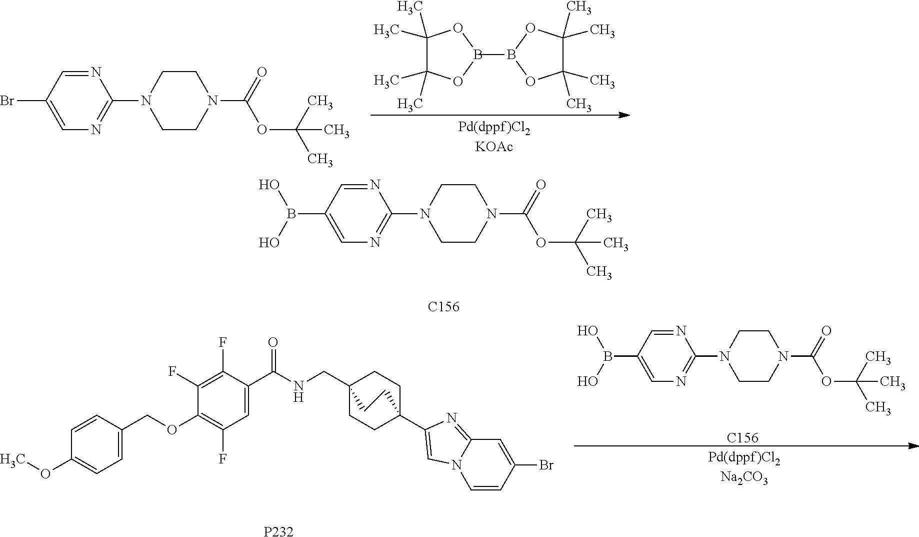

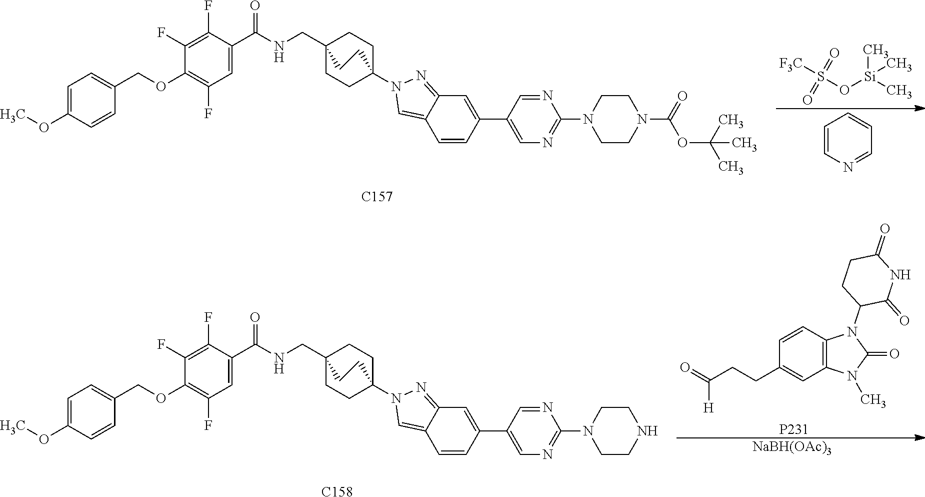

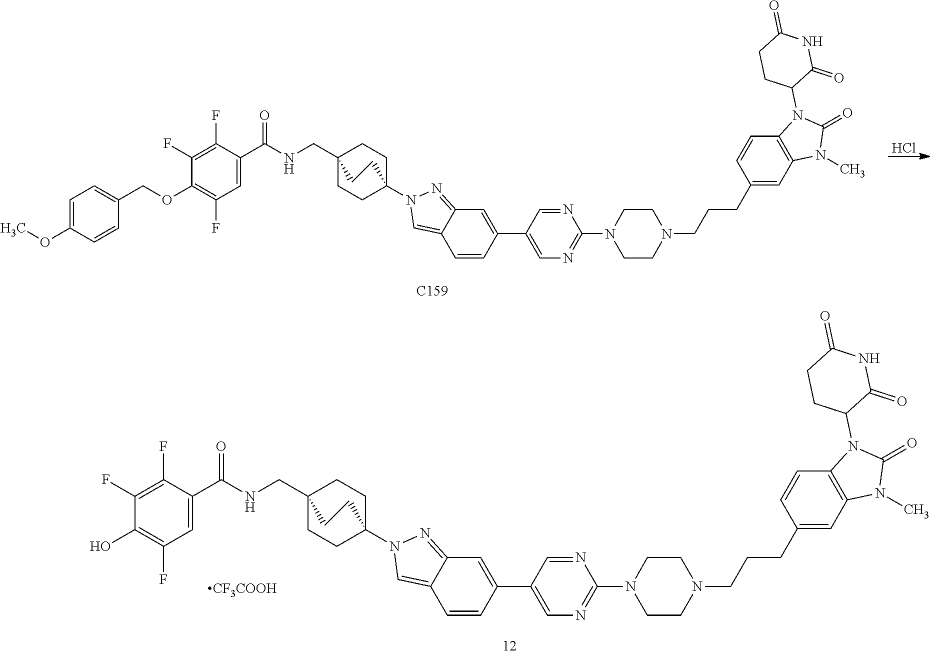 [SMILES: COc1ccc(COc2c(F)cc(C(=O)NC[C@]34CC[C@](n5cc6ccc(-c7cnc(N8CCN(CCCc9ccc%10c(c9)n(C)c(=O)n%10C9CCC(=O)NC9=O)CC8)nc7)cc6n5)(CC3)CC4)c(F)c2F)cc1 ; Cl ; "C159" ; Cn1c(=O)n(C2CCC(=O)NC2=O)c2ccc(CCCN3CCN(c4ncc(-c5ccc6cn([C@]78CC[C@](CNC(=O)c9cc(F)c(O)c(F)c9F)(CC7)CC8)nc6c5)cn4)CC3)cc21 ; "·CF3COOH" ; "12"]

Step 1. Synthesis of {2-[4-(tert-butoxycarbonyl)piperazin-1-yl]pyrimidin-5-yl}boronic acid (C156): [1,1′-Bis(diphenylphosphino)ferrocene]dichloropalladium(II) (1.07 g, 1.46 mmol) and potassium acetate (4.29 g, 43.7 mmol) were added to a solution of tert-butyl 4-(5-bromopyrimidin-2-yl)piperazine-1-carboxylate (5.00 g, 14.6 mmol) and 4,4,4′,4′,5,5,5′,5′-octamethyl-2,2′-bi-1,3,2-dioxaborolane (4.44 g, 17.5 mmol) in 1,4-dioxane (120 mL), and the reaction mixture was stirred at 90° C. After 16 hours, water (100 mL) was added, and the resulting mixture was extracted with ethyl acetate (2×100 mL); the combined organic layers were washed with saturated aqueous sodium chloride solution, dried over sodium sulfate, filtered, and concentrated in vacuo. Silica gel chromatography (Gradient: 5% to 10% methanol in dichloromethane) afforded C156 as a solid. Yield: 4.12 g, 13.4 mmol, 92%. LCMS m/z 309.2 [M+H]⁺. ¹H NMR (400 MHz, DMSO-d₆) δ 8.63 (s, 2H), 8.09 (br s, 2H), 3.78-3.71 (m, 4H), 3.43-3.35 (m, 4H), 1.42 (s, 9H).

Step 2. Synthesis of tert-butyl 4-(5-{2-[4-({2,3,5-trifluoro-4-[(4-methoxyphenyl)methoxy]benzamido}methyl)bicyclo[2.2.2]octan-1-yl]imidazo[1,2-a]pyridin-7-yl}pyrimidin-2-yl)piperazine-1-carboxylate (C157): A mixture of P232 (400 mg, 0.636 mmol), C156 (235 mg, 0.763 mmol), [1,1′-bis(diphenylphosphino)ferrocene]dichloropalladium(II) (93.1 mg, 0.127 mmol), and sodium carbonate (169 mg, 1.59 mmol) in a mixture of 1,4-dioxane (25 mL) and water (2.5 mL) was stirred at 85° C. for 8 hours. The reaction mixture was concentrated in vacuo, and the residue was purified using silica gel chromatography (Gradient: 0% to 100% ethyl acetate in petroleum ether), providing C157. Yield: 360 mg, 0.443 mmol, 70%. LCMS m/z 812.4 [M+H]⁺.

Step 3. Synthesis of 2,3,5-trifluoro-4-[(4-methoxyphenyl)methoxy]-N-[(4-{7-[2-(piperazin-1-yl)pyrimidin-5-yl]imidazo[1,2-a]pyridin-2-yl}bicyclo[2.2.2]octan-1-yl)methyl]benzamide (C158): Trimethylsilyl trifluoromethanesulfonate (394 mg, 1.77 mmol) was added drop-wise to a 0° C. mixture of C157 (360 mg, 0.443 mmol) and pyridine (281 mg, 3.55 mmol) in dichloromethane (30 mL). After the reaction mixture had been stirred at 25° C. for 16 hours, it was treated with aqueous sodium carbonate solution (30 mL) and aqueous sodium bicarbonate solution (20 mL), and then extracted with dichloromethane (2×20 mL). The combined organic layers were washed with saturated aqueous sodium chloride solution, filtered, and concentrated in vacuo. The residue was purified via chromatography on silica gel (Gradient: 0% to 100% ethyl acetate in petroleum ether, then 0% to 20% methanol in dichloromethane) to afford C158 as a yellow solid. Yield: 215 mg, 0.302 mmol, 68%. LCMS m/z 712.3 [M+H]⁺. ¹H NMR (400 MHz, DMSO-d₆) δ 8.83 (s, 2H), 8.47 (br d, J=7.1 Hz, 1H), 8.31 (br t, J=6.3 Hz, 1H), 7.79 (br s, 1H), 7.58 (s, 1H), 7.36 (d, J=8.6 Hz, 2H), 7.35-7.30 (m, 1H), 7.18 (dd, J=7.1, 1.9 Hz, 1H), 6.94 (d, J=8.6 Hz, 2H), 5.22 (s, 2H), 3.80-3.76 (m, 4H), 3.75 (s, 3H), 3.06 (d, J=6.2 Hz, 2H), 2.87-2.80 (m, 4H), 1.88-1.78 (m, 6H), 1.56-1.47 (m, 6H).

Step 4. Synthesis of N-[(4-{7-[2-(4-{3-[1-(2,6-dioxopiperidin-3-yl)-3-methyl-2-oxo-2,3-dihydro-1H-benzimidazol-5-yl]propyl}piperazin-1-yl)pyrimidin-5-yl]imidazo[1,2-a]pyridin-2-yl}bicyclo[2.2.2]octan-1-yl)methyl]-2,3,5-trifluoro-4-[(4-methoxyphenyl)methoxy]benzamide (C159): To a 0° C. solution of P231 (22 mg, 70 μmol) and C158 (49.7 mg, 69.8 μmol) in dimethyl sulfoxide (3 mL) was added sodium triacetoxyborohydride (88.7 mg, 0.419 mmol). The reaction mixture was maintained at 0° C. for 5 minutes, whereupon it was stirred at 25° C. for 2 hours. Water (15 mL) was then added, and the resulting mixture was extracted with ethyl acetate (2×20 mL); the combined organic layers were washed with saturated aqueous sodium chloride solution, dried over sodium sulfate, filtered, and concentrated in vacuo. Silica gel chromatography (Gradient: 0% to 20% methanol in dichloromethane) afforded C159 as a yellow oil. Yield: 50 mg, 49 μmol, 70%. LCMS m/z 1011.5 [M+H]⁺.

Step 5. Synthesis of N-{[4-(7-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-5-yl}imidazo[1,2-a]pyridin-2-yl)bicyclo[2.2.2]octan-1-yl]methyl}-2,3,5-trifluoro-4-hydroxybenzamide, trifluoroacetate salt (12): To a solution of C159 (50 mg, 49 μmol) in dichloromethane (10 mL) was added a solution of hydrogen chloride in 1,4-dioxane (4 M; 1.0 mL, 4.0 mmol). After the reaction mixture had been stirred at 25° C. for 30 minutes, it was concentrated in vacuo and purified via reversed-phase HPLC (Column: Welch Xtimate C18, 30×250 mm, 10 μm; Mobile phase A: water containing 0.1% formic acid; Mobile phase B: acetonitrile; Gradient: 15% to 95% B; Flow rate: 50 mL/minute). Trifluoroacetate salt formation occurred during lyophilization of the fractions, due to trifluoroacetic acid present in the equipment; N-{[4-(7-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-5-yl}imidazo[1,2-a]pyridin-2-yl)bicyclo[2.2.2]octan-1-yl]methyl}-2,3,5-trifluoro-4-hydroxybenzamide, trifluoroacetate salt (12) was isolated as a light-yellow solid. Yield: 3.6 mg, 3.6 μmol, 7%. LCMS m/z 891.4 [M+H]⁺. ¹H NMR (400 MHz, DMSO-d₆), characteristic peaks, integrations are approximate: δ 11.08 (br s, 1H), 8.82 (s, 2H), 8.46 (d, J=7.1 Hz, 1H), 7.80 (br s, 1H), 7.58 (s, 1H), 7.17 (dd, J=7.2, 1.9 Hz, 1H), 7.09-7.06 (m, 1H), 7.03-6.95 (m, 2H), 6.90 (dd, J=8.1, 1.6 Hz, 1H), 5.38-5.30 (m, 1H), 3.85-3.77 (m, 4H), 3.33 (s, 3H), 3.06 (d, J=6.1 Hz, 2H), 2.96-2.83 (m, 1H), 2.77-2.57 (m, 4H), 2.47-2.41 (m, 4H), 2.38-2.31 (m, 2H), 2.04-1.95 (m, 2H), 1.88-1.77 (m, 6H), 1.54-1.44 (m, 6H).

### Example 13

### N-{[(1r,4r)-4-(6-{2-[8-(3-{1-[(3RS)-2,6-Dioxopiperidin-3-yl]-3-methyl-2-oxo-2,3-dihydro-1H-benzimidazol-5-yl}propyl)-5-oxa-2,8-diazaspiro[3.5]nonan-2-yl]pyrimidin-5-yl}-2H-indazol-2-yl)cyclohexyl]methyl}-2,3,5-trifluoro-4-hydroxybenzamide, trifluoroacetate salt (13)

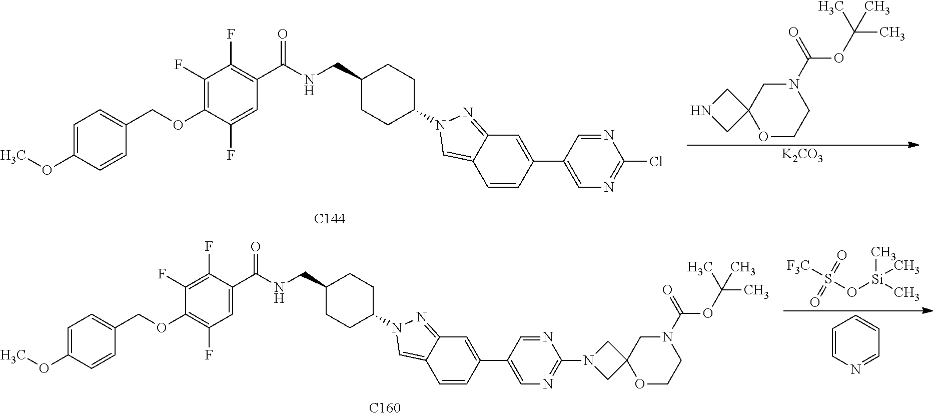

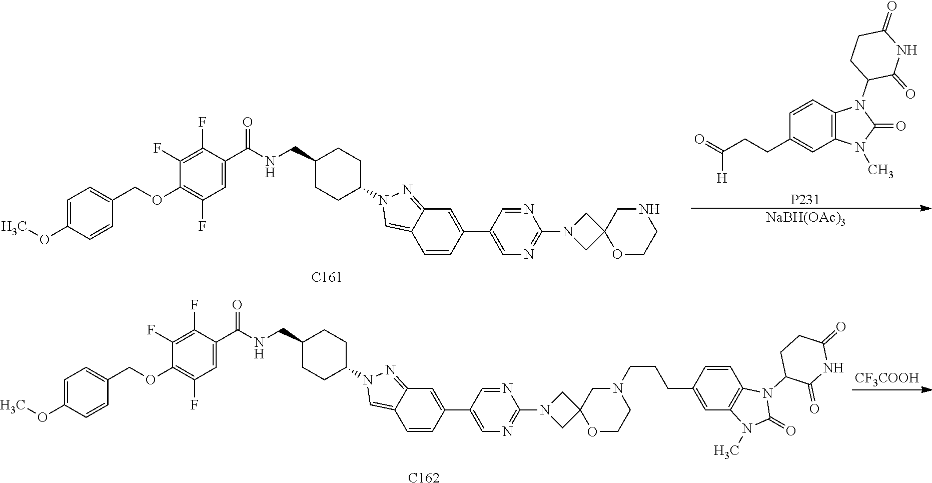

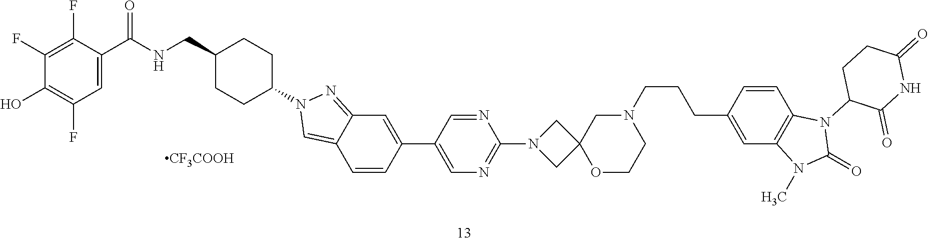 [SMILES: Cn1c(=O)n(C2CCC(=O)NC2=O)c2ccc(CCCN3CCOC4(C3)CN(c3ncc(-c5ccc6cn([C@H]7CC[C@H](CNC(=O)c8cc(F)c(O)c(F)c8F)CC7)nc6c5)cn3)C4)cc21 ; "·CF3COOH" ; "13"]

Step 1. Synthesis of tert-butyl 2-(5-{2-[(1r,4r)-4-({2,3,5-trifluoro-4-[(4-methoxyphenyl)methoxy]benzamido}methyl)cyclohexyl]-2H-indazol-6-yl}pyrimidin-2-yl)-5-oxa-2,8-diazaspiro[3.5]nonane-8-carboxylate (C160): Potassium carbonate (326 mg, 2.36 mmol) was added to a solution of C144 (500 mg, 0.786 mmol) and tert-butyl 5-oxa-2,8-diazaspiro[3.5]nonane-8-carboxylate (197 mg, 0.863 mmol) in N,N-dimethylformamide (5.2 mL), whereupon the reaction mixture was heated at 80° C. overnight. After the reaction mixture had been partitioned between water and ethyl acetate, the aqueous layer was extracted twice with ethyl acetate; the combined organic layers were washed with saturated aqueous sodium chloride solution, dried over sodium sulfate, filtered, and concentrated in vacuo. Purification via silica gel chromatography (Gradient: 0% to 100% ethyl acetate in heptane) provided C160 as a solid. Yield: 493 mg, 0.595 mmol, 76%. LCMS m/z 828.6 [M+H]⁺. ¹H NMR (400 MHz, DMSO-d₆) δ 8.78 (s, 2H), 8.49 (br t, J=5.8 Hz, 1H), 8.41 (s, 1H), 7.85 (br s, 1H), 7.77 (br d, J=8.7 Hz, 1H), 7.39-7.33 (m, 1H), 7.35 (d, J=8.7 Hz, 2H), 7.31 (dd, J=8.7, 1.5 Hz, 1H), 6.94 (d, J=8.7 Hz, 2H), 5.22 (s, 2H), 4.54-4.41 (m, 1H), 3.96 (AB quartet, J_{AB}=9.4 Hz, Δv_{AB}=21.6 Hz, 4H), 3.75 (s, 3H), 3.66-3.61 (m, 2H), 3.58-3.53 (m, 2H), 3.38-3.3 (m, 2H, assumed; partially obscured by water peak), 3.21-3.14 (m, 2H), 2.21-2.12 (m, 2H), 1.98-1.85 (m, 4H), 1.72-1.60 (m, 1H), 1.42 (s, 9H), 1.29-1.16 (m, 2H).

Step 2. Synthesis of 2,3,5-trifluoro-4-[(4-methoxyphenyl)methoxy]-N-{[(1r,4r)-4-{6-[2-(5-oxa-2,8-diazaspiro[3.5]nonan-2-yl)pyrimidin-5-yl]-2H-indazol-2-yl}cyclohexyl]methyl}benzamide (C161): Trimethylsilyl trifluoromethanesulfonate (0.430 mL, 2.38 mmol) was slowly added to a 0° C. solution of C160 (492 mg, 0.594 mmol) and pyridine (0.383 mL, 4.74 mmol) in dichloromethane (20 mL), whereupon the reaction mixture was allowed to warm to room temperature and stir overnight. LCMS analysis indicated conversion to C161: LCMS m/z 728.6 [M+H]⁺. The reaction mixture was cooled to 0° C. and diluted with saturated aqueous sodium bicarbonate solution; the aqueous layer was extracted three times with dichloromethane. The combined organic layers were washed sequentially with saturated aqueous sodium bicarbonate solution and saturated aqueous sodium chloride solution, dried over sodium sulfate, filtered, and concentrated in vacuo to provide C161 (530 mg), a portion of which was progressed directly to the following step. ¹H NMR (400 MHz, DMSO-d₆) δ 8.75 (s, 2H), 8.49 (br t, J=6 Hz, 1H), 8.41 (s, 1H), 7.84 (s, 1H), 7.76 (d, J=8.6 Hz, 1H), 7.40-7.27 (m, 4H), 6.94 (d, J=8.6 Hz, 2H), 5.22 (s, 2H), 4.54-4.41 (m, 1H), 3.93 (AB quartet, J_{AB}=9.3 Hz, Δv_{AB}=44.7 Hz, 4H), 3.75 (s, 3H), 3.61-3.54 (m, 2H), 3.21-3.14 (m, 2H), 2.86 (s, 2H), 2.70-2.63 (m, 2H), 2.21-2.11 (m, 2H), 1.98-1.84 (m, 4H), 1.72-1.60 (m, 1H), 1.30-1.14 (m, 2H).

Step 3. Synthesis of N-{[(1r,4r)-4-{6-[2-(8-{3-[1-(2,6-dioxopiperidin-3-yl)-3-methyl-2-oxo-2,3-dihydro-1H-benzimidazol-5-yl]propyl}-5-oxa-2,8-diazaspiro[3.5]nonan-2-yl)pyrimidin-5-yl]-2H-indazol-2-yl}cyclohexyl]methyl}-2,3,5-trifluoro-4-[(4-methoxyphenyl)methoxy]benzamide (C162): A mixture of C161 (from the previous step; 106 mg, 50.119 mmol) and P231 (90%, 56.1 mg, 0.160 mmol) in tetrahydrofuran (2.4 mL) was maintained at room temperature for 30 minutes, whereupon sodium triacetoxyborohydride (123 mg, 0.580 mmol) was added, and the reaction mixture was heated at 50° C. After 30 minutes, the heat was removed, and stirring was continued overnight at room temperature. LCMS analysis indicated the presence of C162: LCMS m/z 1027.8 [M+H]⁺. After the reaction mixture had been diluted with dichloromethane, aqueous sodium bicarbonate solution was added, and the aqueous layer was extracted three times with dichloromethane. The combined organic layers were washed with saturated aqueous sodium chloride solution, dried over sodium sulfate, filtered, and concentrated in vacuo to afford C162 as a white solid (167 mg). Most of this material was taken directly to the following step. ¹H NMR (400 MHz, DMSO-d₆), characteristic peaks, aliphatic integrations are approximate: δ 11.07 (s, 1H), 8.75 (s, 2H), 8.49 (br t, J=6 Hz, 1H), 8.41 (br s, 1H), 7.84 (br s, 1H), 7.76 (d, J=8.7 Hz, 1H), 7.39-7.33 (m, 1H), 7.35 (d, J=8.7 Hz, 2H), 7.30 (dd, J=8.7, 1.5 Hz, 1H), 7.05 (br s, 1H), 7.00 (d, half of AB quartet, J=8.0 Hz, 1H), 6.94 (d, J=8.6 Hz, 2H), 6.88 (dd, component of ABX system, J=8.0, 1.5 Hz, 1H), 5.32 (dd, J=12.7, 5.4 Hz, 1H), 5.22 (s, 2H), 4.53-4.42 (m, 1H), 3.96 (AB quartet, J_{AB}=9.4 Hz, Δv_{AB}=27.0 Hz, 4H), 3.75 (s, 3H), 3.70-3.63 (m, 2H), 3.31 (s, 3H), 3.21-3.14 (m, 2H), 2.94-2.82 (m, 1H), 2.75-2.60 (m, 4H), 2.60-2.54 (m, 2H), 2.40-2.29 (m, 3H), 2.21-2.11 (m, 2H), 2.04-1.85 (m, 5H), 1.71-1.60 (m, 1H), 1.30-1.14 (m, 4H).

Step 4. Synthesis of N-{[(1r,4r)-4-(6-{2-[8-(3-{1-[(3RS)-2,6-dioxopiperidin-3-yl]-3-methyl-2-oxo-2,3-dihydro-1H-benzimidazol-5-yl}propyl)-5-oxa-2,8-diazaspiro[3.5]nonan-2-yl]pyrimidin-5-yl}-2H-indazol-2-yl)cyclohexyl]methyl}-2,3,5-trifluoro-4-hydroxybenzamide, trifluoroacetate salt (13): Trifluoroacetic acid (16.7 mg, 0.146 mmol) was added to a solution of C162 (from the previous step; 150 mg, 50.107 mmol) in dichloromethane (5 mL). After the reaction mixture had been stirred at room temperature for 15 minutes, solvent was removed under a stream of nitrogen. Purification via reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 5% to 95% B over 8.5 minutes, then 95% B over 1.5 minutes; Flow rate: 25 mL/minute) afforded N-{[(1r,4r)-4-(6-{2-[8-(3-{1-[(3RS)-2,6-dioxopiperidin-3-yl]-3-methyl-2-oxo-2,3-dihydro-1H-benzimidazol-5-yl}propyl)-5-oxa-2,8-diazaspiro[3.5]nonan-2-yl]pyrimidin-5-yl}-2H-indazol-2-yl)cyclohexyl]methyl}-2,3,5-trifluoro-4-hydroxybenzamide, trifluoroacetate salt (13) as an oil. Yield: 28.2 mg, 27.6 μmol, 26% over 3 steps. LCMS m/z 907.7 [M+H]⁺. ¹H NMR (600 MHz, DMSO-d₆), characteristic peaks, integrations are approximate: δ 11.08 (s, 1H), 8.78 (s, 2H), 8.41 (s, 1H), 8.34 (br t, J=6 Hz, 1H), 7.85 (br s, 1H), 7.77 (d, J=8.7 Hz, 1H), 7.34-7.26 (m, 2H), 7.07 (br s, 1H), 7.04 (d, J=8.0 Hz, 1H), 6.92 (d, J=8.0 Hz, 1H), 5.37-5.30 (m, 1H), 4.52-4.43 (m, 1H), 3.33 (s, 3H), 3.21-3.15 (m, 2H), 2.94-2.84 (m, 1H), 2.72-2.65 (m, 2H), 2.67-2.58 (m, 1H), 2.20-2.11 (m, 2H), 2.06-1.95 (m, 3H), 1.95-1.86 (m, 4H), 1.71-1.62 (m, 1H), 1.28-1.19 (m, 2H).

### Example 14

### N-{[(1r,4r)-4-(6-{6-[4-(2-{1-[(3RS)-2,6-Dioxopiperidin-3-yl]-3-methyl-2-oxo-2,3-dihydro-1H-benzimidazol-5-yl}ethyl)piperazin-1-yl]pyrazin-2-yl}-2H-indazol-2-yl)cyclohexyl]methyl}-2,3,5-trifluoro-4-hydroxybenzamide, trifluoroacetate salt (14)

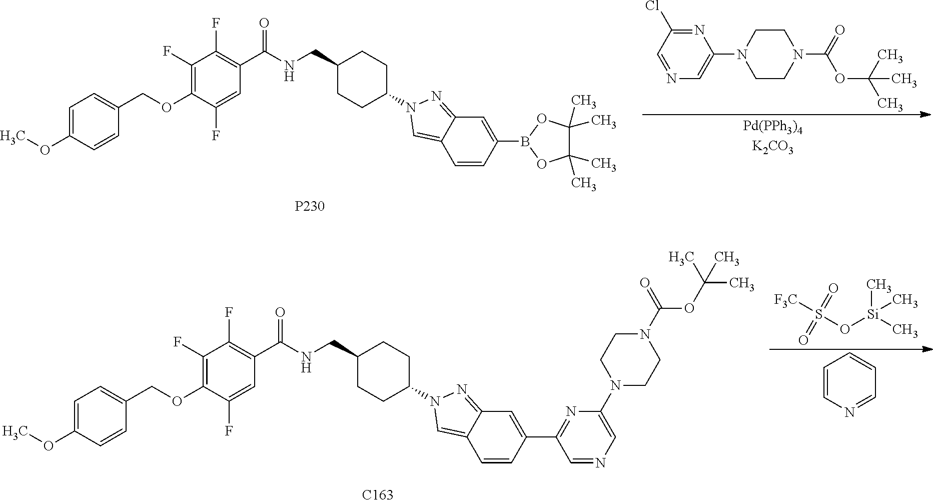

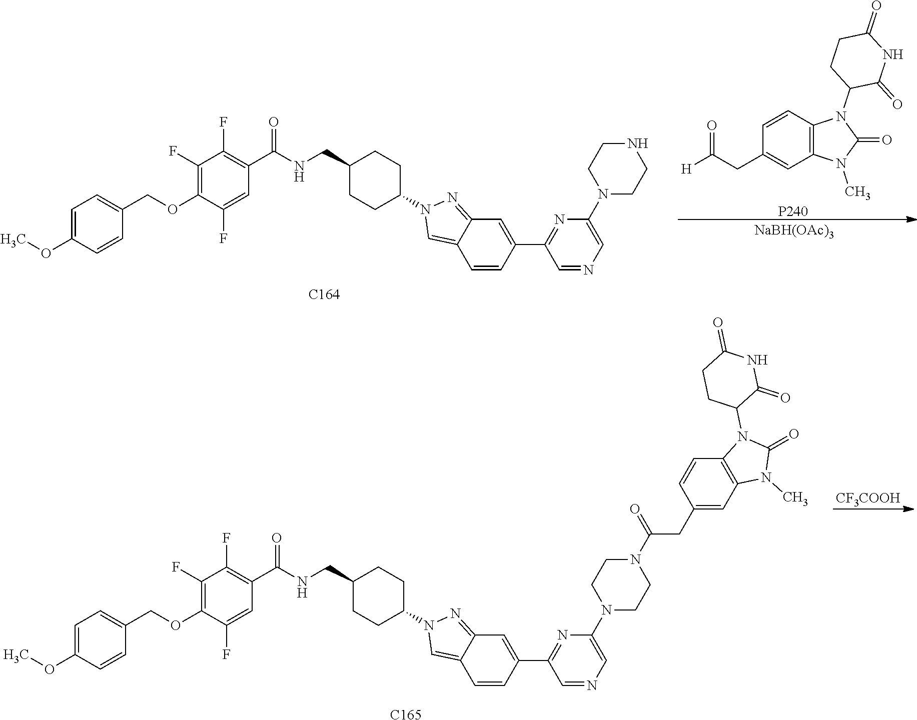

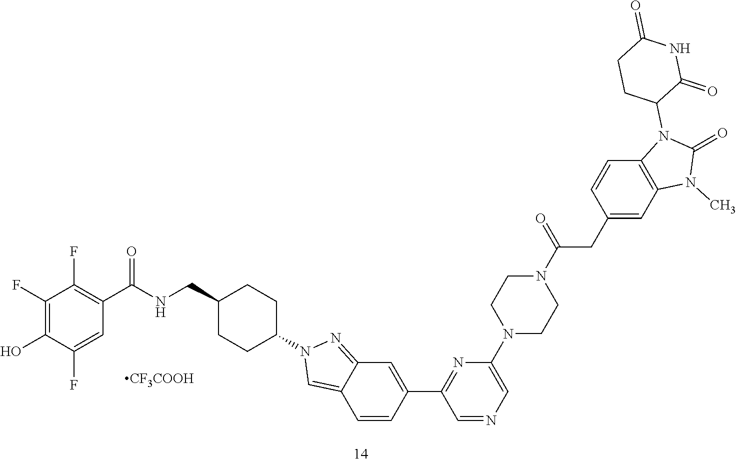

Step 1. Synthesis of tert-butyl 4-(6-{2-[(1r,4r)-4-({2,3,5-trifluoro-4-[(4-methoxyphenyl)methoxy]benzamido}methyl)cyclohexyl]-2H-indazol-6-yl}pyrazin-2-yl)piperazine-1-carboxylate (C163): A solution of P230 (100 mg, 0.154 mmol), tert-butyl 4-(6-chloropyrazin-2-yl)piperazine-1-carboxylate (46.0 mg, 0.154 mmol), potassium carbonate (64.0 mg, 0.463 mmol), and tetrakis(triphenylphosphine)palladium(0) (18.0 mg, 15.6 μmol) in a mixture of 1,4-dioxane (2.0 mL) and water (0.5 mL) was purged with nitrogen for 5 minutes, whereupon it was stirred at 90° C. for 20 hours. After the reaction mixture had been cooled and concentrated in vacuo, the residue was taken up in ethyl acetate and filtered through a pad of diatomaceous earth. The filter pad was washed with ethyl acetate and then with 10% methanol in dichloromethane; the combined filtrates were dried over sodium sulfate, filtered, and concentrated under reduced pressure, affording C163 as a black solid (168 mg). This material was progressed directly to the following step. LCMS m/z 786.5 [M+H]⁺.

Step 2. Synthesis of 2,3,5-trifluoro-4-[(4-methoxyphenyl)methoxy]-N-{[(1r,4r)-4-{6-[6-(piperazin-1-yl)pyrazin-2-yl]-2H-indazol-2-yl}cyclohexyl]methyl}benzamide (C164): Trimethylsilyl trifluoromethanesulfonate (122 μL, 0.674 mmol) was added drop-wise to a 0° C. solution of C163 (from the previous step; 168 mg, 50.154 mmol) and pyridine (111 μL, 1.37 mmol) in dichloromethane (7 mL). The reaction mixture was allowed to stir in the cooling bath overnight, by which time the bath had warmed to 17° C. LCMS analysis indicated conversion to C164: LCMS m/z 686.4 [M+H]⁺. After the reaction mixture had been cooled to 0° C. and stirred for 10 minutes, saturated aqueous sodium bicarbonate solution (10 mL) was slowly added, and stirring was continued for 10 minutes. The aqueous layer was extracted with dichloromethane (3×15 mL), and the combined organic layers were dried over sodium sulfate, filtered, and concentrated in vacuo to provide C164 as a black solid (150 mg). A portion of this material was used in the following step.

Step 3. Synthesis of N-{[(1r,4r)-4-{6-[6-(4-{2-[1-(2,6-dioxopiperidin-3-yl)-3-methyl-2-oxo-2,3-dihydro-1H-benzimidazol-5-yl]ethyl}piperazin-1-yl)pyrazin-2-yl]-2H-indazol-2-yl}cyclohexyl]methyl}-2,3,5-trifluoro-4-[(4-methoxyphenyl)methoxy]benzamide (C165): To a mixture of P240 (83%, 17 mg, 47 μmol) and C164 (from the previous step; 55 mg, 556 μmol) in dichloromethane (1 mL) was added acetic acid (3 μL, 50 μmol). After the mixture had been stirred at room temperature for 30 minutes, sodium triacetoxyborohydride (20 mg, 94 μmol) was added in one portion and the reaction mixture was stirred at room temperature overnight. It was then diluted with dichloromethane and saturated aqueous sodium bicarbonate solution, and the aqueous layer was extracted three times with dichloromethane; the combined organic layers were dried over sodium sulfate, filtered, and concentrated in vacuo, affording C165 as a dark solid (44 mg). LCMS m/z 969.4 [M−H]⁻.

Step 4. Synthesis of N-{[(1r,4r)-4-(6-{6-[4-(2-{1-[(3RS)-2,6-dioxopiperidin-3-yl]-3-methyl-2-oxo-2,3-dihydro-1H-benzimidazol-5-yl}ethyl)piperazin-1-yl]pyrazin-2-yl}-2H-indazol-2-yl)cyclohexyl]methyl}-2,3,5-trifluoro-4-hydroxybenzamide, trifluoroacetate salt (14): Trifluoroacetic acid (0.50 mL, 6.5 mmol) was added drop-wise to a 0° C. solution of C165 (from the previous step; 44 mg, 545 μmol) in dichloromethane (1 mL). The reaction mixture was allowed to stir at room temperature for 20 minutes, whereupon it was concentrated in vacuo and purified via reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 15% to 55% B over 8.5 minutes, then 55% to 95% B over 0.5 minutes; Flow rate: 25 mL/minute), providing N-{[(1r,4r)-4-(6-{6-[4-(2-{1-[(3RS)-2,6-dioxopiperidin-3-yl]-3-methyl-2-oxo-2,3-dihydro-1H-benzimidazol-5-yl}ethyl)piperazin-1-yl]pyrazin-2-yl}-2H-indazol-2-yl)cyclohexyl]methyl}-2,3,5-trifluoro-4-hydroxybenzamide, trifluoroacetate salt (14). Yield: 6.1 mg, 6.3 μmol, 14% over 2 steps. LCMS m/z 851.6 [M+H]⁺. ¹H NMR (400 MHz, DMSO-d₆), characteristic peaks, integrations are approximate: δ 11.10 (s, 1H), 8.65 (s, 1H), 8.45 (s, 1H), 8.40 (br s, 2H), 8.37-8.33 (m, 2H), 7.82-7.77 (m, 2H), 7.29 (ddd, J=11.0, 6.2, 2.4 Hz, 1H), 7.14 (br s, 1H), 7.04 (AB quartet, upfield doublet is broadened, J_{AB}=8.1 Hz, Δv_{AB}=66.0 Hz, 2H), 5.35 (dd, J=12.9, 5.4 Hz, 1H), 4.73-4.41 (m, 2H), 3.34 (s, 3H), 3.20-3.16 (m, 2H), 3.11-3.05 (m, 2H), 2.94-2.85 (m, 1H), 2.74-2.66 (m, 1H), 2.66-2.60 (m, 1H), 2.21-2.13 (m, 2H), 2.04-1.97 (m, 1H), 1.97-1.87 (m, 4H), 1.72-1.63 (m, 1H), 1.29-1.18 (m, 2H).

### Example 15

### N-{[4-(3-{6-[4-(3-{1-[(3RS)-2,6-Dioxopiperidin-3-yl]-3-methyl-2-oxo-2,3-dihydro-1H-benzimidazol-5-yl}propyl)piperazin-1-yl]pyridazin-3-yl}-1,2,4-oxadiazol-5-yl)bicyclo[2.2.2]octan-1-yl]methyl}-2,3,5-trifluoro-4-hydroxybenzamide, trifluoroacetate salt (15)

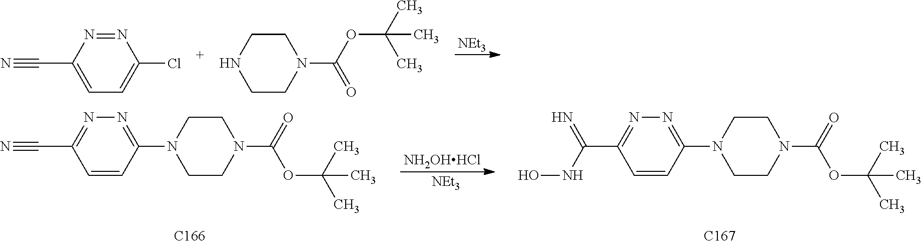

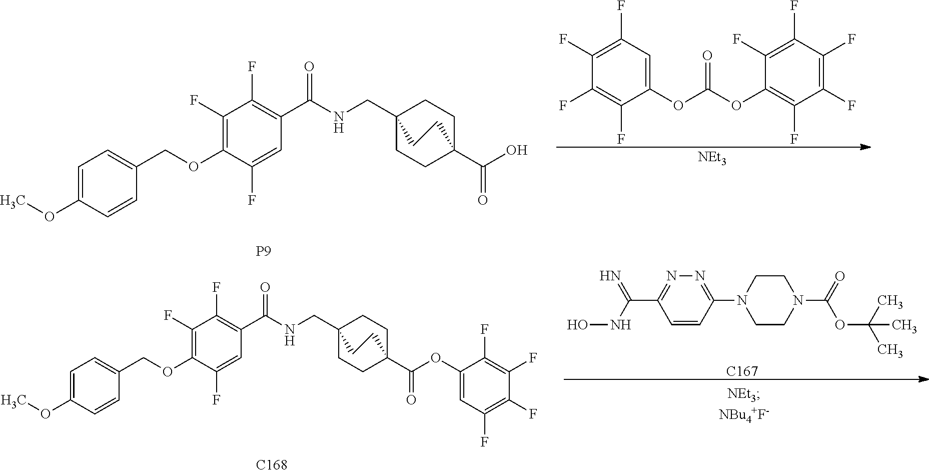

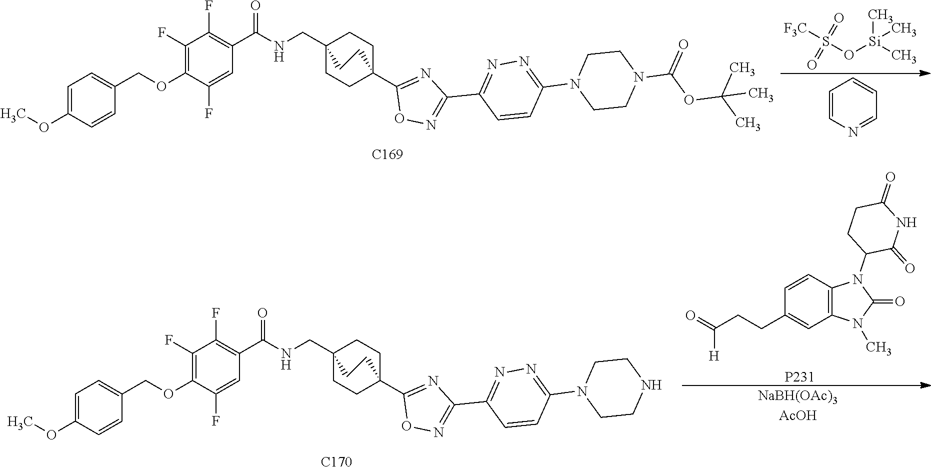

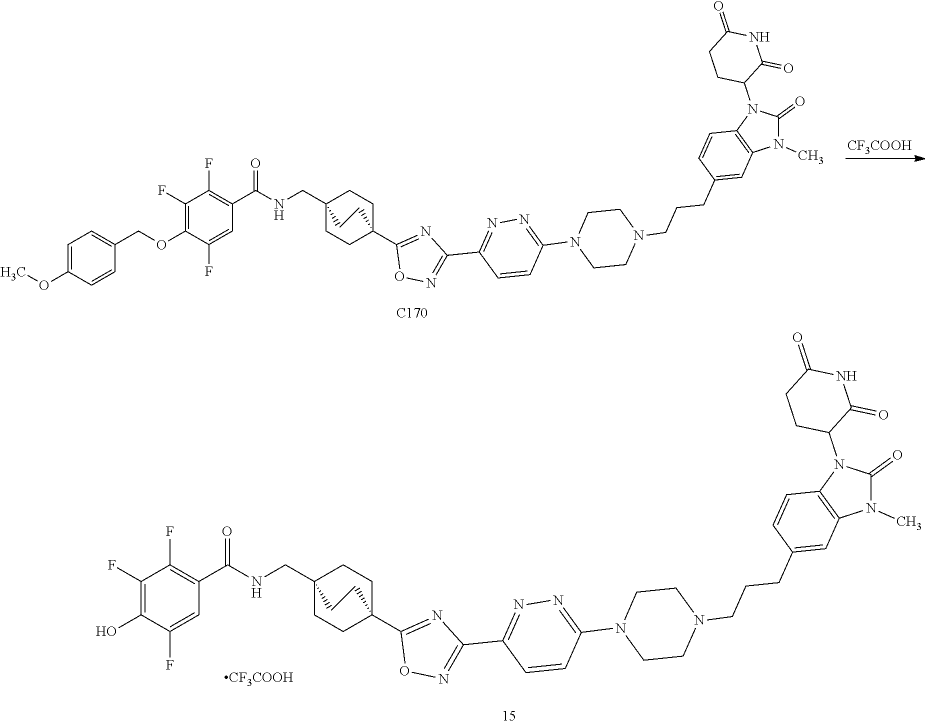

Step 1. Synthesis of tert-butyl 4-(6-cyanopyridazin-3-yl)piperazine-1-carboxylate (C166): A solution of tert-butyl piperazine-1-carboxylate (294 mg, 1.58 mmol) and triethylamine (0.400 mL, 2.87 mmol) in N,N-dimethylformamide (5 mL) was stirred for 10 minutes, whereupon 6-chloropyridazine-3-carbonitrile (200 mg, 1.43 mmol) was added and the reaction mixture was gradually heated to 60° C. After two hours, the reaction mixture was cooled to room temperature and added to a mixture of water (100 mL) and saturated aqueous sodium chloride solution (10 mL). The resulting suspension was extracted with ethyl acetate (3×60 mL) and the combined organic layers were dried over sodium sulfate, filtered, and concentrated in vacuo. Silica gel chromatography (Gradient: 0% to 100% ethyl acetate in heptane) afforded C166 as a solid. Yield: 300 mg, 1.04 mmol, 73%. LCMS m/z 234.2 [(M-2-methylprop-1-ene)+H]⁺. ¹H NMR (400 MHz, DMSO-d₆) δ 7.90 (d, J=9.7 Hz, 1H), 7.34 (d, J=9.7 Hz, 1H), 3.80-3.73 (m, 4H), 3.50-3.43 (m, 4H), 1.43 (s, 9H).

Step 2. Synthesis of tert-butyl 4-[6-(N-hydroxycarbamimidoyl)pyridazin-3-yl]piperazine-1-carboxylate (C167): To a solution of C166 (285 mg, 0.985 mmol) and hydroxylamine hydrochloride (75.3 mg, 1.08 mmol) in methanol (5.0 mL) was added triethylamine (0.275 mL, 1.97 mmol), whereupon the reaction mixture was stirred at room temperature overnight. It was then concentrated under reduced pressure, and the residue was dissolved in ethyl acetate (60 mL) and washed sequentially with water (3×60 mL) and saturated aqueous sodium chloride solution (60 mL). Concentration in vacuo afforded C167 as a solid. Yield: 325 mg, assumed quantitative. LCMS m/z 323.3 [M+H]⁺. ¹H NMR (400 MHz, DMSO-d₆) δ 9.89 (s, 1H), 7.72 (d, J=9.6 Hz, 1H), 7.28 (d, J=9.7 Hz, 1H), 5.87 (br s, 2H), 3.67-3.60 (m, 4H), 3.49-3.42 (m, 4H), 1.43 (s, 9H).

Step 3. Synthesis of pentafluorophenyl 4-({2,3,5-trifluoro-4-[(4-methoxyphenyl)methoxy]benzamido}methyl)bicyclo[2.2.2]octane-1-carboxylate (C168): A solution of P9 (1.94 g, 4.06 mmol) and bis(pentafluorophenyl) carbonate (1.76 g, 4.46 mmol) in acetonitrile (15 mL) was treated with triethylamine (1.24 mL, 8.90 mmol), whereupon the reaction mixture was stirred at room temperature for 3 hours before being concentrated in vacuo. Purification via silica gel chromatography (Gradient: 5% to 80% ethyl acetate in heptane) provided C168 as a solid. Yield: 2.34 g, 3.64 mmol, 90%. LCMS m/z 644.3 [M+H]⁺. ¹H NMR (400 MHz, DMSO-d₆) δ 8.35 (br t, J=6.3 Hz, 1H), 7.35 (d, J=8.7 Hz, 2H), 7.35-7.30 (m, 1H), 6.94 (d, J=8.6 Hz, 2H), 5.21 (s, 2H), 3.75 (s, 3H), 3.05 (d, J=6.3 Hz, 2H), 1.95-1.85 (m, 6H), 1.55-1.45 (m, 6H).

Step 4. Synthesis of tert-butyl 4-(6-{5-[4-({2,3,5-trifluoro-4-[(4-methoxyphenyl)methoxy]benzamido}methyl)bicyclo[2.2.2]octan-1-yl]-1,2,4-oxadiazol-3-yl}pyridazin-3-yl)piperazine-1-carboxylate (C169): A mixture of C168 (599 mg, 0.931 mmol) and C167 (300 mg, 0.931 mmol) in tetrahydrofuran (5 mL) was treated with dimethyl sulfoxide (0.5 mL) to provide a solution. After addition of triethylamine (0.259 mL, 1.86 mmol), the reaction mixture was stirred at room temperature for 2 hours, whereupon triethylamine (0.259 mL, 1.86 mmol) was again added and the reaction mixture was allowed to stir overnight. Solvent was removed in vacuo, and the residue was taken up in ethyl acetate (200 mL) and washed with a mixture of water and saturated aqueous sodium chloride solution (1:1, 3×150 mL). The organic layer was dried over sodium sulfate, filtered, and concentrated in vacuo to afford the acyl intermediate as a solid (740 mg). LCMS m/z 782.5 [M+H]⁺. This material was dissolved in tetrahydrofuran (8.0 mL) and treated with a solution of tetrabutylammonium fluoride in tetrahydrofuran (1.0 M; 2.84 mL, 2.84 mmol), whereupon the reaction mixture was stirred at room temperature for 4 hours. After solvent had been removed in vacuo, the residue was purified via silica gel chromatography (Gradient: 0% to 30% methanol in dichloromethane), affording C169 as a solid. Yield: 460 mg, 0.602 mmol, 65%. LCMS m/z 764.5 [M+H]⁺. ¹H NMR (400 MHz, DMSO-d₆) δ 8.37 (br t, J=6.3 Hz, 1H), 7.89 (d, J=9.6 Hz, 1H), 7.37 (d, J=9.4 Hz, 1H), 7.37-7.31 (m, 1H), 7.35 (d, J=8.6 Hz, 2H), 6.94 (d, J=8.7 Hz, 2H), 5.22 (s, 2H), 3.75 (s, 3H), 3.75-3.70 (m, 4H), 3.52-3.45 (m, 4H), 3.07 (d, J=6.2 Hz, 2H), 2.02-1.92 (m, 6H), 1.59-1.50 (m, 6H), 1.43 (s, 9H).

Step 5. Synthesis of 2,3,5-trifluoro-4-[(4-methoxyphenyl)methoxy]-N-[(4-{3-[6-(piperazin-1-yl)pyridazin-3-yl]-1,2,4-oxadiazol-5-yl}bicyclo[2.2.2]octan-1-yl)methyl]benzamide (C170): Trimethylsilyl trifluoromethanesulfonate (180 μL, 0.995 mmol) was added drop-wise to a 0° C. solution of C169 (190 mg, 0.249 mmol) and pyridine (163 μL, 2.02 mmol) in dichloromethane (10 mL). After the reaction mixture had stirred overnight in the cooling bath, the bath temperature had risen to 16° C.; the reaction mixture was cooled to 0° C., whereupon saturated aqueous sodium bicarbonate solution (7 mL) was slowly added. The resulting mixture was stirred for 10 minutes, and the aqueous layer was extracted with dichloromethane (3×10 mL); the combined organic layers were dried over sodium sulfate, filtered, and concentrated in vacuo to provide C170 as a light-yellow solid (209 mg), a portion of which was progressed to the following step. LCMS m/z 664.4 [M+H]⁺. ¹H NMR (400 MHz, chloroform-d), characteristic peaks, integrations are approximate; δ 7.99-7.90 (m, 1H), 7.63-7.55 (m, 1H), 7.34 (d, J=8.6 Hz, 2H), 7.02-6.92 (m, 1H), 6.88 (d, J=8.6 Hz, 2H), 6.62-6.51 (m, 1H), 5.24 (s, 2H), 4.04-3.95 (m, 2H), 3.81 (s, 3H), 3.31 (d, J=6.2 Hz, 2H), 3.28-3.21 (m, 3H), 2.14-2.04 (m, 6H), 1.67-1.57 (m, 6H).

Step 6. Synthesis of N-[(4-{3-[6-(4-{3-[1-(2,6-dioxopiperidin-3-yl)-3-methyl-2-oxo-2,3-dihydro-1H-benzimidazol-5-yl]propyl}piperazin-1-yl)pyridazin-3-yl]-1,2,4-oxadiazol-5-yl}bicyclo[2.2.2]octan-1-yl)methyl]-2,3,5-trifluoro-4-[(4-methoxyphenyl)methoxy]benzamide (C171): Acetic acid (3 μL, 50 μmol) was added to a mixture of P231 (85%, 17 mg, 46 μmol) and C170 (from the previous step; 30 mg, 536 μmol) in dichloromethane (1.0 mL). After the resulting solution had been stirred at room temperature for 30 minutes, sodium triacetoxyborohydride (19 mg, 90 μmol) was added in one portion and the reaction mixture was stirred at room temperature overnight. Additional dichloromethane was added, followed by saturated aqueous sodium bicarbonate solution, and the aqueous layer was extracted twice with dichloromethane. The combined organic layers were dried over sodium sulfate, filtered, and concentrated in vacuo to provide C171 as an off-white solid (58 mg); this material was taken directly to the following step. LCMS m/z 963.8 [M+H]⁺.

Step 7. Synthesis of N-{[4-(3-{6-[4-(3-{1-[(3RS)-2,6-dioxopiperidin-3-yl]-3-methyl-2-oxo-2,3-dihydro-1H-benzimidazol-5-yl}propyl)piperazin-1-yl]pyridazin-3-yl}-1,2,4-oxadiazol-5-yl)bicyclo[2.2.2]octan-1-yl]methyl}-2,3,5-trifluoro-4-hydroxybenzamide, trifluoroacetate salt (15): Trifluoroacetic acid (0.50 mL, 6.5 mmol) was added drop-wise to a 0° C. solution of C171 (from the previous step; 58 mg, 536 μmol) in dichloromethane (1.0 mL). After the reaction mixture had been stirred at room temperature for 20 minutes, it was concentrated in vacuo. Purification via reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 10% to 50% B over 8.5 minutes, then 50% to 95% B over 0.5 minutes, then 95% B for 1.0 minute; Flow rate: 25 mL/minute) afforded N-{[4-(3-{6-[4-(3-{1-[(3RS)-2,6-dioxopiperidin-3-yl]-3-methyl-2-oxo-2,3-dihydro-1H-benzimidazol-5-yl}propyl)piperazin-1-yl]pyridazin-3-yl}-1,2,4-oxadiazol-5-yl)bicyclo[2.2.2]octan-1-yl]methyl}-2,3,5-trifluoro-4-hydroxybenzamide, trifluoroacetate salt (15). Yield: 22.5 mg, 23.5 μmol, 65% over 3 steps. LCMS m/z 843.6 [M+H]⁺. ¹H NMR (600 MHz, DMSO-d₆), characteristic peaks: δ 11.09 (s, 1H), 8.20 (br t, J=6 Hz, 1H), 7.97 (d, J=9.5 Hz, 1H), 7.49 (d, J=9.7 Hz, 1H), 7.27 (ddd, J=11.0, 6.2, 2.2 Hz, 1H), 7.09 (br s, 1H), 6.99 (AB quartet, upfield doublet is broadened, J_{AB}=8.0 Hz, Δv_{AB}=71.6 Hz, 2H), 5.34 (dd, J=12.9, 5.4 Hz, 1H), 4.74-4.48 (m, 2H), 3.34 (s, 3H), 3.20-3.11 (m, 3H), 3.08 (d, J=6.3 Hz, 2H), 2.94-2.85 (m, 1H), 2.74-2.59 (m, 4H), 2.08-1.92 (m, 9H), 1.59-1.51 (m, 6H).

### Example 16

### N-{[(1r,4r)-4-{5-[4-(3-{1-[(3RS)-2,6-Dioxopiperidin-3-yl]-3-methyl-2-oxo-2,3-dihydro-1H-benzimidazol-5-yl}propyl)piperazin-1-yl]-2H-pyrazolo[4,3-b]pyridin-2-yl}cyclohexyl]methyl}-2,3,5-trifluoro-4-hydroxybenzamide, formate salt (16)

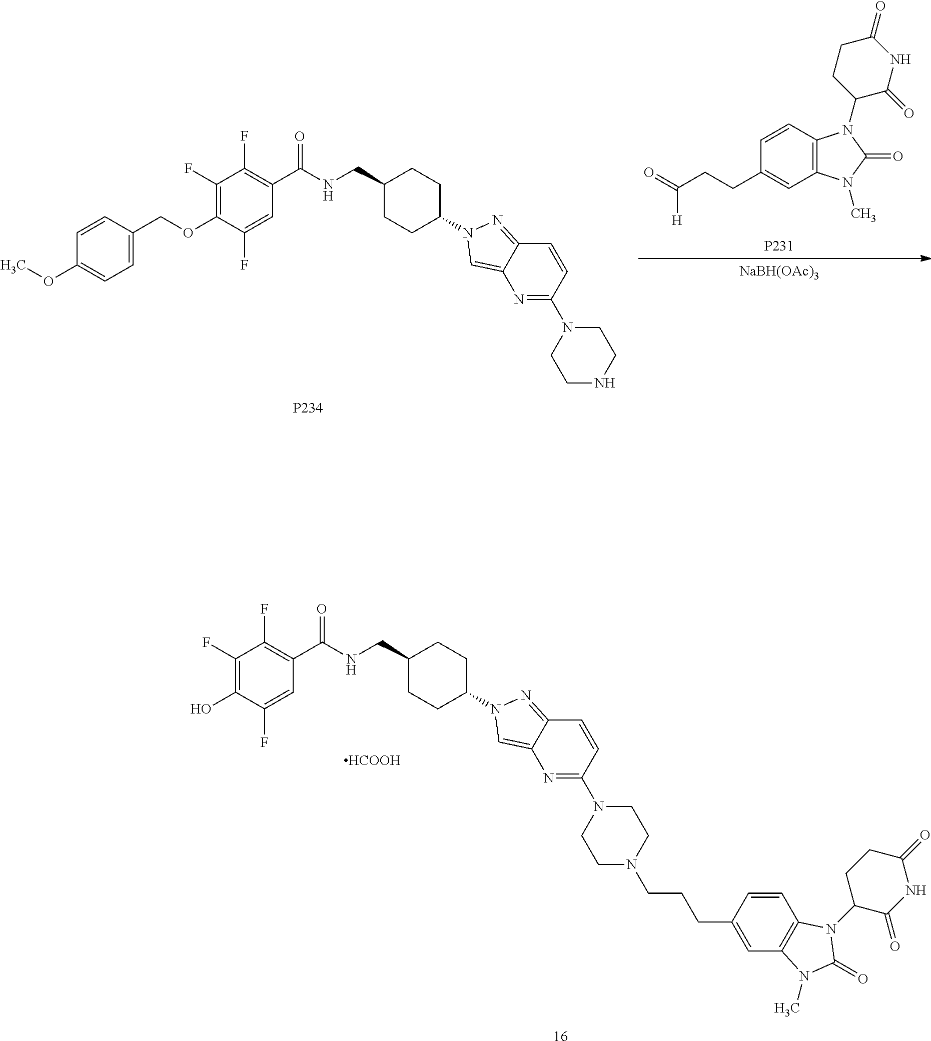

To a solution of P231 (20 mg, 63 μmol) and P234 (38.6 mg, 63.4 μmol) in dimethyl sulfoxide (5 mL) was added sodium triacetoxyborohydride (40.3 mg, 0.190 mmol). After the reaction mixture had been stirred at 15° C. for 1 hour, water (30 mL) was added, and the resulting mixture was extracted with ethyl acetate (2×20 mL). The combined organic layers were washed with saturated aqueous sodium chloride solution, dried over sodium sulfate, filtered, and concentrated in vacuo; silica gel chromatography (Gradient: 0% to 10% methanol in dichloromethane), followed by reversed-phase HPLC (Column: Waters XBridge C18, 19×150 mm, 5 μm; Mobile phase A: water containing 0.05% formic acid; Mobile phase B: acetonitrile; Gradient: 20% to 30% B; Flow rate: 20 mL/minute), afforded N-{[(1r,4r)-4-{5-[4-(3-{1-[(3RS)-2,6-dioxopiperidin-3-yl]-3-methyl-2-oxo-2,3-dihydro-1H-benzimidazol-5-yl}propyl)piperazin-1-yl]-2H-pyrazolo[4,3-b]pyridin-2-yl}cyclohexyl]methyl}-2,3,5-trifluoro-4-hydroxybenzamide, formate salt (16) as a solid. Yield: 5.3 mg, 6.4 μmol, 10%. LCMS m/z 788.3 [M+H]⁺. ¹H NMR (400 MHz, DMSO-d₆), characteristic peaks, some integrations are approximate: δ 11.08 (s, 1H), 8.21-8.07 (m, 3H), 7.82 (d, J=9.5 Hz, 1H), 7.26-7.18 (m, 1H), 7.10-7.03 (m, 2H), 6.95 (AB quartet, J_{AB}=8.0 Hz, Δv_{AB}=45.1 Hz, 2H), 5.34 (dd, J=12.9, 5.3 Hz, 1H), 4.40-4.28 (m, 1H), 3.56-3.46 (m, 4H), 3.33 (s, 3H, assumed; overlaps with water peak), 3.19-3.12 (m, 2H), 2.96-2.83 (m, 1H), 2.78-2.57 (m, 4H, assumed; partially obscured by solvent peak), 2.42-2.34 (m, 2H), 2.16-2.05 (m, 2H), 1.94-1.75 (m, 5H), 1.71-1.58 (m, 1H).

### Example 17

### N-{[(1r,4r)-4-(5-{2-[4-(3-{1-[(3RS)-2,6-Dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)cyclohexyl]methyl}-2,3,5-trifluoro-4-hydroxybenzamide, trifluoroacetate salt (17)

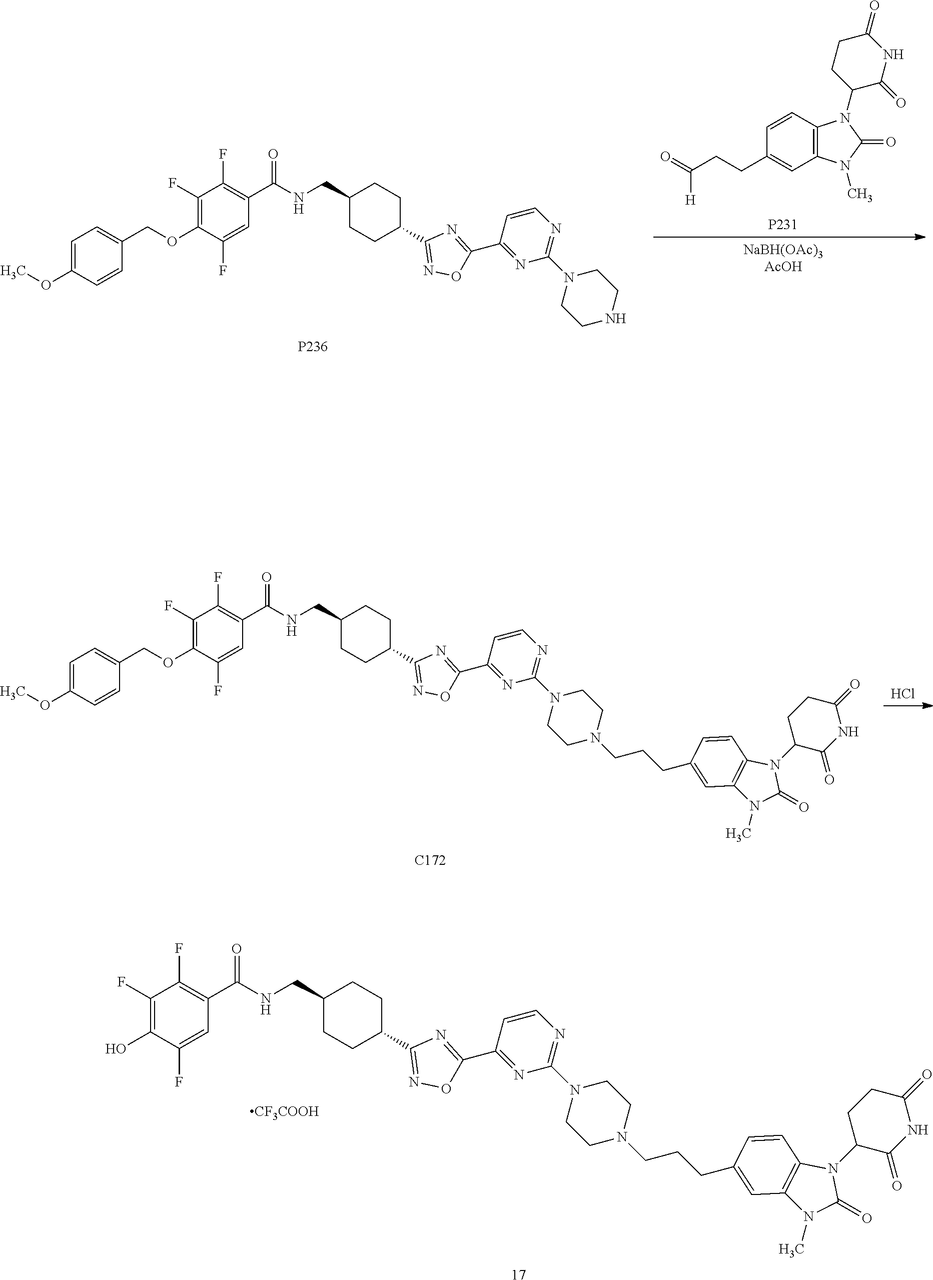

Step 1. Synthesis of N-[(4-{5-[2-(4-{3-[1-(2,6-dioxopiperidin-3-yl)-3-methyl-2-oxo-2,3-dihydro-1H-benzimidazol-5-yl]propyl}piperazin-1-yl)pyrimidin-4-yl]-1,2,4-oxadiazol-3-yl}cyclohexyl)methyl]-2,3,5-trifluoro-4-[(4-methoxyphenyl)methoxy]benzamide (C172): Acetic acid (6.9 μL, 0.12 mmol) was added to a solution of P236 (95%, 81 mg, 0.12 mmol) and P231 (90%, 46.5 mg, 0.133 mmol) in dichloromethane (2.4 mL), whereupon the reaction mixture was heated at 40° C. After 15 minutes, it was cooled to room temperature; sodium triacetoxyborohydride (51.2 mg, 0.241 mmol) was added, and the reaction mixture was heated at 40° C. for an additional 30 minutes. It was again allowed to cool to room temperature, then treated with saturated aqueous sodium bicarbonate solution. The aqueous layer was extracted three times with dichloromethane, and the combined organic layers were washed with saturated aqueous sodium chloride solution, dried over sodium sulfate, filtered, and concentrated in vacuo to afford C172 as a yellow tacky solid (133 mg). This material was taken directly to the next step. LCMS m/z 937.8 [M+H]⁺.

Step 2. Synthesis of N-{[(1r,4r)-4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)cyclohexyl]methyl}-2,3,5-trifluoro-4-hydroxybenzamide, trifluoroacetate salt (17): A solution of hydrogen chloride in 1,4-dioxane (4.0 M; 1.1 mL, 4.4 mmol) was slowly added, over approximately 1 minute, to a 0° C. solution of C172 (from the previous step; 133.0 mg, 50.12 mmol) in dichloromethane (3 mL). After the reaction mixture had been stirred for 15 minutes at 0° C., followed by 4 hours at room temperature, the reaction solution was decanted away from the solid that had formed. The solid was then stirred with acetonitrile (3 mL), and the liquid was again decanted off, whereupon propan-2-ol (3 mL) was added and stirring was continued for 20 minutes. The resulting gel was diluted with acetonitrile (5 mL) and stirred, whereupon the mixture was filtered through a 0.45 μm nylon filter; the filtrate was concentrated in vacuo, and the resulting solid was stirred with acetone (3 mL) for 15 minutes, isolated via filtration, and rinsed with a small amount of acetone to afford N-{[(1r,4r)-4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)cyclohexyl]methyl}-2,3,5-trifluoro-4-hydroxybenzamide, hydrochloride salt (17, hydrochloride salt) as a pale-yellow solid. Yield: 39 mg, 46 μmol, 38% over 2 steps.

The filter paper was rinsed with propan-2-ol, and the rinse was combined with the acetone filtrates and concentrated in vacuo. The residue was purified via reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 20% to 40% B over 8.5 minutes, then 40% to 95% B over 0.5 minutes; Flow rate: 25 mL/minute), providing N-{[(1r,4r)-4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)cyclohexyl]methyl}-2,3,5-trifluoro-4-hydroxybenzamide, trifluoroacetate salt (17). Additional yield: 19.2 mg, 20.6 μmol, 17% over 2 steps; total yield: 55% over 2 steps. LCMS m/z 817.6 [M+H]⁺. ¹H NMR (600 MHz, DMSO-d₆), characteristic peaks: δ 11.41 (br s, 1H), 11.09 (s, 1H), 9.72 (br s, 1H), 8.75 (d, J=4.9 Hz, 1H), 8.32-8.28 (m, 1H), 7.44 (d, J=4.9 Hz, 1H), 7.28 (ddd, J=11.0, 6.3, 2.3 Hz, 1H), 7.08 (d, J=1.6 Hz, 1H), 7.05 (d, half of AB quartet, J=8.0 Hz, 1H), 6.92 (dd, component of ABX system, J=8.0, 1.6 Hz, 1H), 5.35 (dd, J=13.0, 5.5 Hz, 1H), 4.85-4.66 (m, 2H), 3.71-3.54 (m, 2H), 3.34 (s, 3H, assumed; partially obscured by water peak), 3.19-3.03 (m, 5H or 6H), 2.95-2.83 (m, 2H), 2.74-2.65 (m, 3H), 2.65-2.60 (m, 1H), 2.12-2.06 (m, 2H), 2.06-1.96 (m, 2H), 1.90-1.84 (m, 2H), 1.65-1.56 (m, 1H), 1.56-1.46 (m, 2H), 1.19-1.09 (m, 2H).

### Example 18

### N-{[4-(2-{2-[4-(3-{1-[(3RS)-2,6-Dioxopiperidin-3-yl]-3-methyl-2-oxo-2,3-dihydro-1H-benzimidazol-5-yl}propyl)piperazin-1-yl]pyrimidin-4-yl}-1,3-oxazol-5-yl)bicyclo[2.2.2]octan-1-yl]methyl}-2,3,5-trifluoro-4-hydroxybenzamide (18)

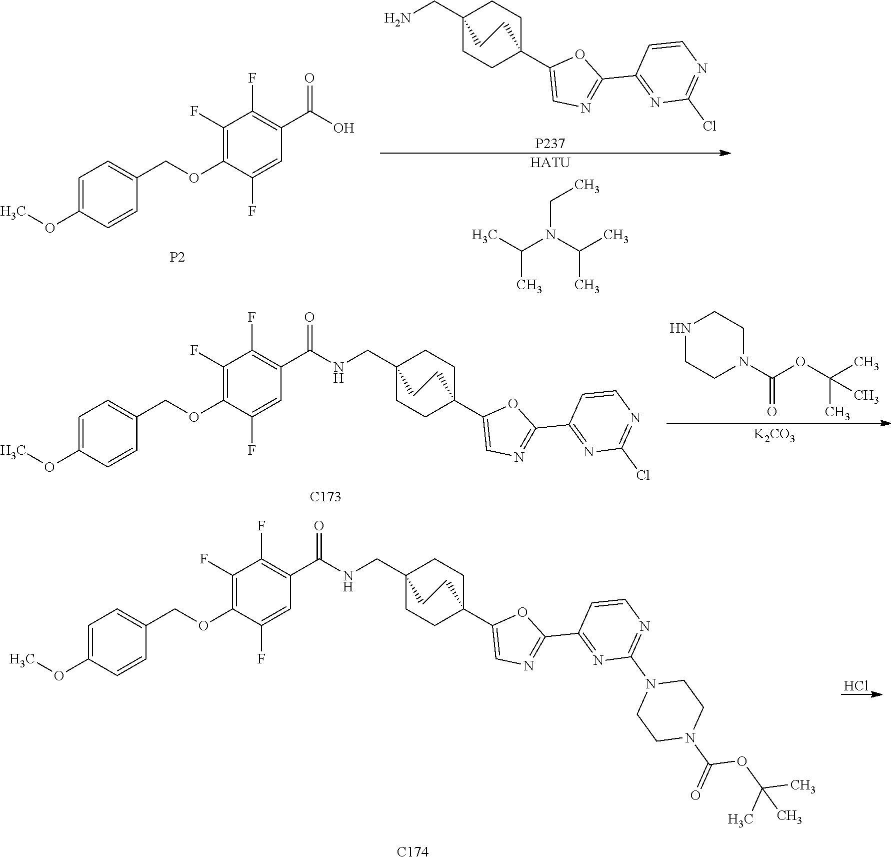

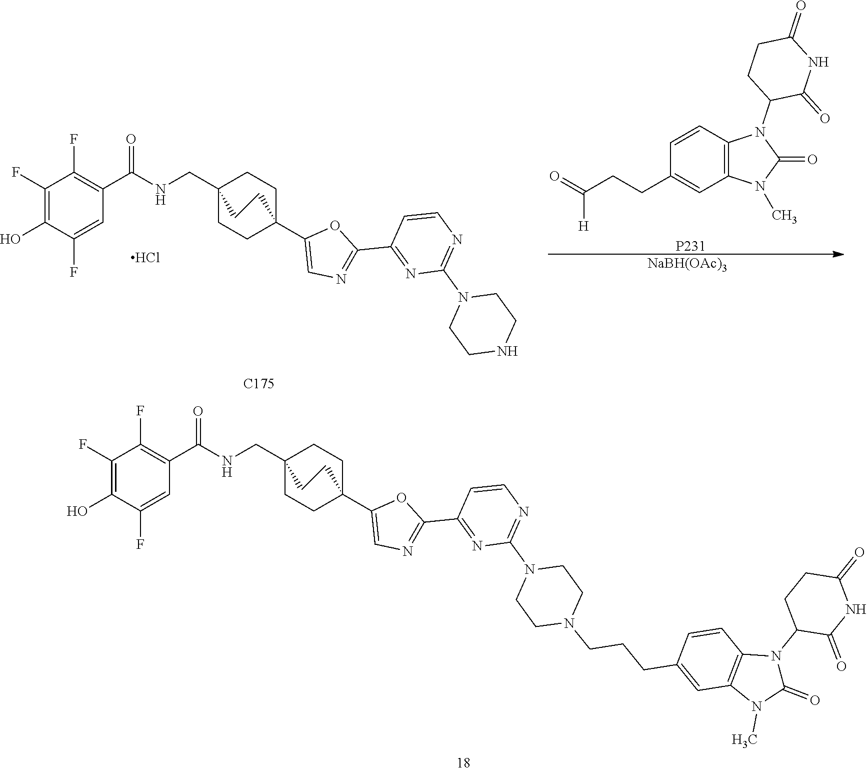

Step 1. Synthesis of N-({4-[2-(2-chloropyrimidin-4-yl)-1,3-oxazol-5-yl]bicyclo[2.2.2]octan-1-yl}methyl)-2,3,5-trifluoro-4-[(4-methoxyphenyl)methoxy]benzamide (C173): N,N-Diisopropylethylamine (62 mg, 0.48 mmol) was added to a solution of P2 (50 mg, 0.16 mmol) and O-(7-azabenzotriazol-1-yl)-N,N,N′N′-tetramethyluronium hexafluorophosphate (HATU; 73.1 mg, 0.192 mmol) in N,N-dimethylformamide (4 mL). After the mixture had been stirred at 25° C. for 2 minutes, P237 (55 mg, 0.17 mmol) was added, and the reaction mixture was stirred at 25° C. for 1 hour. Water (30 mL) was added, and the resulting mixture was extracted with ethyl acetate (2×30 mL); the combined organic layers were washed with saturated aqueous sodium chloride solution, dried over sodium sulfate, filtered, and concentrated in vacuo. Purification via silica gel chromatography (Gradient: 40% to 80% ethyl acetate in petroleum ether) afforded C173 as a solid. Yield: 67 mg, 0.11 mmol, 69%. LCMS m/z 613.2 [M+H]⁺.

Step 2. Synthesis of tert-butyl 4-(4-{5-[4-({2,3,5-trifluoro-4-[(4-methoxyphenyl)methoxy]benzamido}methyl)bicyclo[2.2.2]octan-1-yl]-1,3-oxazol-2-yl}pyrimidin-2-yl)piperazine-1-carboxylate (C174): To a mixture of tert-butyl piperazine-1-carboxylate (29.6 mg, 0.159 mmol) and potassium carbonate (58.6 mg, 0.424 mmol) in acetonitrile (10 mL) was added C173 (65 mg, 0.11 mmol). The reaction mixture was stirred at 80° C. for 16 hours, whereupon it was filtered; the filtrate was concentrated in vacuo and purified using chromatography on silica gel (Gradient: 30% to 100% ethyl acetate in petroleum ether) to afford C174 as a white solid. Yield: 62 mg, 81 μmol, 74%. ¹H NMR (400 MHz, DMSO-d₆) δ 8.51 (d, J=5.0 Hz, 1H), 8.35 (br t, J=6 Hz, 1H), 7.37-7.30 (m, 1H), 7.36 (d, J=8.6 Hz, 2H), 7.18 (d, J=5.0 Hz, 1H), 7.09 (s, 1H), 6.94 (d, J=8.7 Hz, 2H), 5.22 (s, 2H), 3.82-3.76 (m, 4H), 3.75 (s, 3H), 3.47-3.39 (m, 4H), 3.06 (d, J=6.2 Hz, 2H), 1.87-1.76 (m, 6H), 1.56-1.47 (m, 6H), 1.43 (s, 9H).

Step 3. Synthesis of 2,3,5-trifluoro-4-hydroxy-N-[(4-{2-[2-(piperazin-1-yl)pyrimidin-4-yl]-1,3-oxazol-5-yl}bicyclo[2.2.2]octan-1-yl)methyl]benzamide, hydrochloride salt (C175): To a solution of C174 (60 mg, 79 μmol) in dichloromethane (4 mL) was added a solution of hydrogen chloride in 1,4-dioxane (4 M; 1 mL, 4 mmol). After the mixture had been stirred at 25° C. for 2 hours, it was concentrated in vacuo, affording C175 as a white solid. Yield: 32 mg, 55 μmol, 70%. LCMS m/z 543.2 [M+H]⁺.

Step 4. Synthesis of N-{[4-(2-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,3-oxazol-5-yl)bicyclo[2.2.2]octan-1-yl]methyl}-2,3,5-trifluoro-4-hydroxybenzamide (18): Sodium triacetoxyborohydride (46.9 mg, 0.221 mmol) was added to a solution of C175 (30 mg, 52 μmol), N,N-diisopropylethylamine (10.7 mg, 82.8 μmol), and P231 (20.9 mg, 66.3 μmol) in dichloromethane (5 mL). The reaction mixture was stirred at 25° C. for 2 hours, whereupon it was concentrated in vacuo and purified via silica gel chromatography (Gradient: 0% to 25% methanol in dichloromethane), followed by reversed-phase HPLC (Column: Waters XBridge C18, 19×150 mm, 5 μm; Mobile phase A: water containing 0.1% formic acid; Mobile phase B: acetonitrile; Gradient: 27% to 37% B; Flow rate: 20 mL/minute), to provide N-{[4-(2-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,3-oxazol-5-yl)bicyclo[2.2.2]octan-1-yl]methyl}-2,3,5-trifluoro-4-hydroxybenzamide (18) as a white solid. Yield: 9.5 mg, 11 μmol, 21%. LCMS m/z 842.4 [M+H]⁺. ¹H NMR (400 MHz, DMSO-d₆) δ 11.11 (s, 1H), 8.48 (d, J=4.9 Hz, 1H), 8.25 (br s, 1H), 7.43-7.30 (m, 1H), 7.14 (d, J=5.0 Hz, 1H), 7.09-7.00 (m, 3H), 7.01 (d, half of AB quartet, J=8.0 Hz, 1H), 6.90 (dd, component of ABX system, J=8.1, 1.5 Hz, 1H), 5.34 (dd, J=12.6, 5.4 Hz, 1H), 3.85-3.75 (m, 4H), 3.33 (s, 3H, assumed; overlaps with water peak), 3.06 (d, J=6.2 Hz, 2H), 2.96-2.84 (m, 1H), 2.77-2.57 (m, 4H), 2.47-2.40 (m, 4H, assumed; partially obscured by solvent peak), 2.38-2.30 (m, 2H), 2.05-1.94 (m, 1H), 1.87-1.73 (m, 8H), 1.56-1.44 (m, 6H).

### Example 19

### N-{[(1r,4r)-4-{6-[2-(4-{3-[3-(2,4-Dioxo-1,3-diazinan-1-yl)imidazo[1,2-a]pyridin-7-yl]propyl}piperazin-1-yl)pyrimidin-5-yl]-2H-indazol-2-yl}cyclohexyl]methyl}-2,3,5-trifluoro-4-hydroxybenzamide, trifluoroacetate salt (19)

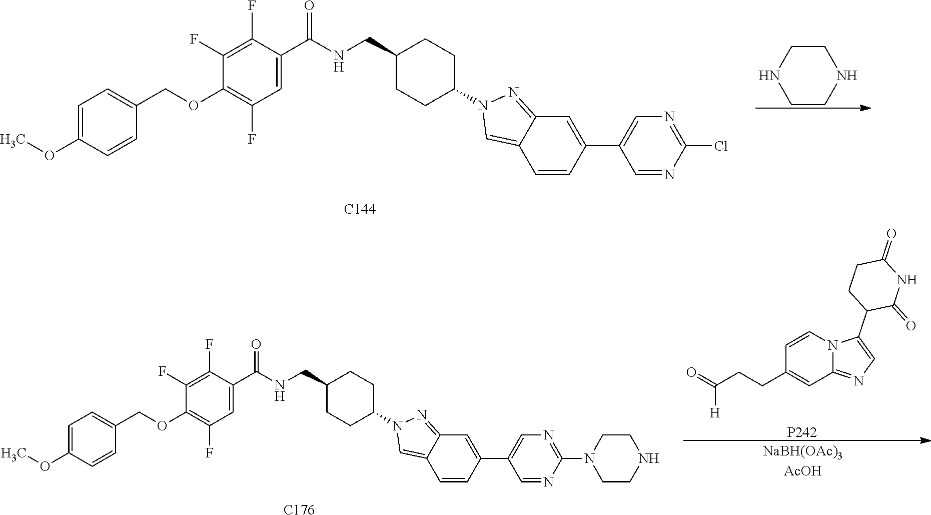

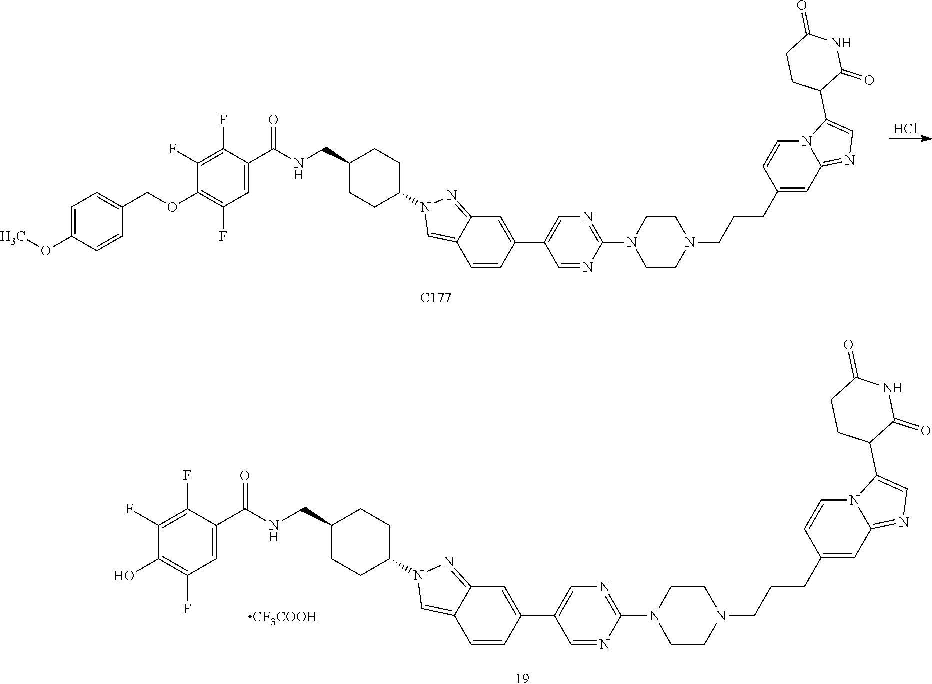

Step 1. Synthesis of 2,3,5-trifluoro-4-[(4-methoxyphenyl)methoxy]-N-{[(1r,4r)-4-{6-[2-(piperazin-1-yl)pyrimidin-5-yl]-2H-indazol-2-yl}cyclohexyl]methyl}benzamide (C176): A solution of C144 (219 mg, 0.344 mmol) and piperazine (123 mg, 1.43 mmol) in N,N-dimethylformamide (1.4 mL) was heated at 45° C. for 30 minutes, whereupon the reaction mixture was cooled to room temperature and treated with water (approximately 5 mL). After the resulting mixture had been vigorously stirred (1500 rpm) for 30 minutes, the solid was collected by filtration. The reaction flask was rinsed with water (approximately 5 mL), and this was used to wash the filter cake. The collected solid was taken up in tetrahydrofuran (25 mL) and stirred for 15 minutes, whereupon the resulting solution was dried over sodium sulfate, filtered, and concentrated in vacuo to afford C176 as a white solid. Yield: 214 mg, 0.312 mmol, 91%. LCMS m/z 686.5 [M+H]⁺.

Step 2. Synthesis of N-{[(1r,4r)-4-{6-[2-(4-{3-[3-(2,4-dioxo-1,3-diazinan-1-yl)imidazo[1,2-a]pyridin-7-yl]propyl}piperazin-1-yl)pyrimidin-5-yl]-2H-indazol-2-yl}cyclohexyl]methyl}-2,3,5-trifluoro-4-[(4-methoxyphenyl)methoxy]benzamide (C177): A mixture of P242 (33 mg, 0.12 mmol) and C176 (53 mg, 77 μmol) in dichloromethane (0.8 mL) and dimethyl sulfoxide (0.2 mL) was treated with acetic acid (5.8 μL, 0.10 mmol), whereupon it was stirred at room temperature for 30 minutes. After addition of sodium triacetoxyborohydride (43.4 mg, 0.205 mmol) in one portion, the reaction mixture was stirred at 40° C. for 30 minutes, cooled to room temperature, treated with methanol (0.2 mL), and stirred for an additional 20 minutes. The resulting mixture was concentrated under reduced pressure, and the residue was partitioned between saturated aqueous sodium bicarbonate solution and dichloromethane; the aqueous layer was extracted twice with dichloromethane, and the combined organic layers were concentrated in vacuo to provide C177 as an oily solid (120 mg). The bulk of this material was progressed to the following step. LCMS m/z 956.6 [M+H]⁺.

Step 3. Synthesis of N-{[(1r,4r)-4-{6-[2-(4-{3-[3-(2,4-dioxo-1,3-diazinan-1-yl)imidazo[1,2-a]pyridin-7-yl]propyl}piperazin-1-yl)pyrimidin-5-yl]-2H-indazol-2-yl}cyclohexyl]methyl}-2,3,5-trifluoro-4-hydroxybenzamide, trifluoroacetate salt (19): To a solution of C177 (from the previous step; 98 mg, 563 μmol) in methanol (1 mL) was added a solution of hydrogen chloride in 1,4-dioxane (4 M; 1 mL, 4 mmol). After the reaction mixture had been stirred for 25 minutes at room temperature, a solution of hydrogen chloride in 1,4-dioxane (4 M; 0.5 mL, 2 mmol) was again added, and stirring was continued for 18 hours at room temperature. After removal of solvent in vacuo, the residue was purified via reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 20% to 30% B over 8.5 minutes, then 30% to 95% B over 0.5 minutes; Flow rate: 25 mL/minute) to afford N-{[(1r,4r)-4-{6-[2-(4-{3-[3-(2,4-dioxo-1,3-diazinan-1-yl)imidazo[1,2-a]pyridin-7-yl]propyl}piperazin-1-yl)pyrimidin-5-yl]-2H-indazol-2-yl}cyclohexyl]methyl}-2,3,5-trifluoro-4-hydroxybenzamide, trifluoroacetate salt (19). Yield: 8.2 mg, 8.6 μmol, 14% over 2 steps. LCMS m/z 836.9 [M+H]⁺. ¹H NMR (600 MHz, DMSO-d₆), characteristic peaks: δ 10.83 (s, 1H), 8.86 (s, 2H), 8.69-8.62 (m, 1H), 8.42 (s, 1H), 8.37-8.32 (m, 1H), 8.02 (br s, 1H), 7.89 (s, 1H), 7.78 (d, J=8.7 Hz, 1H), 7.74 (br s, 1H), 7.37-7.27 (m, 3H), 4.88-4.66 (m, 2H), 4.54-4.43 (m, 1H), 3.88-3.80 (m, 2H), 2.92-2.80 (m, 4H), 2.21-2.07 (m, 4H), 1.97-1.86 (m, 4H), 1.72-1.62 (m, 1H), 1.29-1.17 (m, 2H).

### Example 20

### N-{[(1r,4r)-4-{6-[4-(4-{1-[3-(2,4-Dioxo-1,3-diazinan-1-yl)-4-methylbenzoyl]piperidin-4-yl}butyl)piperazin-1-yl]-2H-indazol-2-yl}cyclohexyl]methyl}-2,3,5-trifluoro-4-hydroxybenzamide (20)

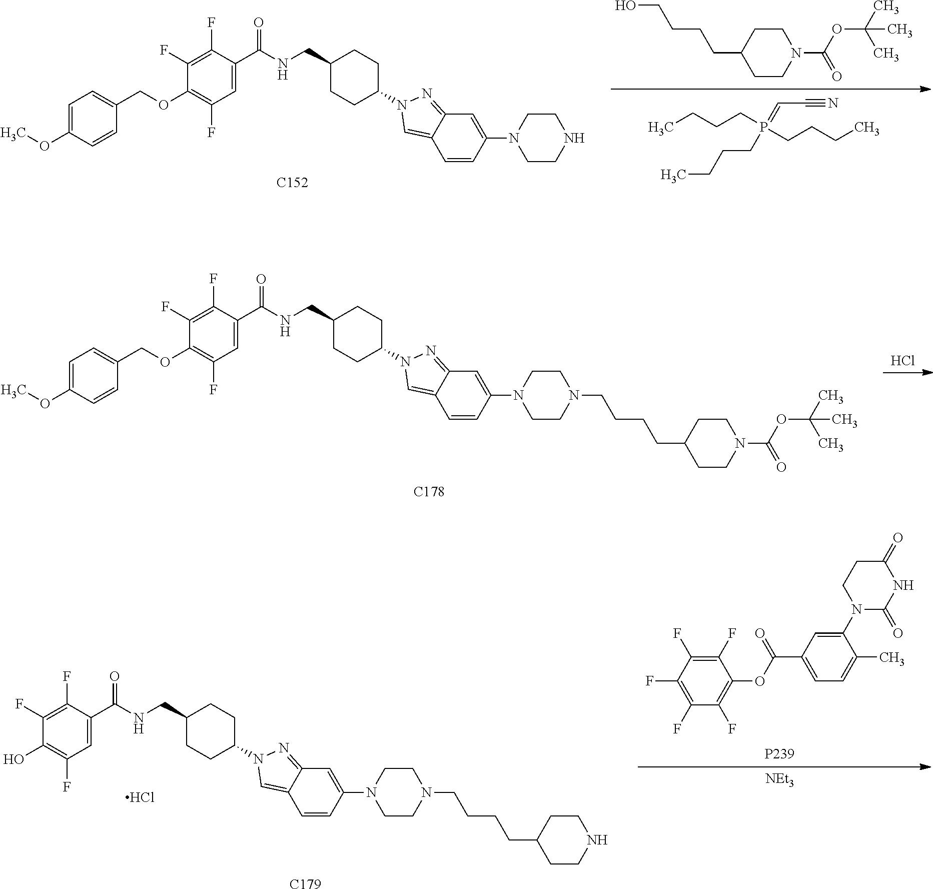

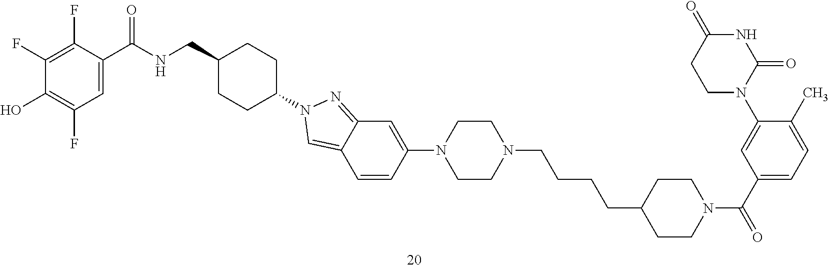 [SMILES: Cc1ccc(C(=O)N2CCC(CCCCN3CCN(c4ccc5cn([C@H]6CC[C@H](CNC(=O)c7cc(F)c(O)c(F)c7F)CC6)nc5c4)CC3)CC2)cc1N1CCC(=O)NC1=O ; "20"]

Step 1. Synthesis of tert-butyl 4-[4-(4-{2-[(1r,4r)-4-({2,3,5-trifluoro-4-[(4-methoxyphenyl)methoxy]benzamido}methyl)cyclohexyl]-2H-indazol-6-yl}piperazin-1-yl)butyl]piperidine-1-carboxylate (C178): To a solution of C152 (200 mg, 0.329 mmol) and tert-butyl 4-(4-hydroxybutyl)piperidine-1-carboxylate (169 mg, 0.657 mmol) in toluene (2 mL) was added (tributyl-λ⁵-phosphanylidene)acetonitrile (317 mg, 1.31 mmol), whereupon the reaction mixture was stirred at 110° C. for 16 hours. After solvent had been removed via concentration in vacuo, the residue was purified using chromatography on silica gel (Eluent: 20:1 dichloromethane/methanol) to provide C178 as a yellow solid. Yield: 220 mg, 0.260 mmol, 79%. LCMS m/z 847.5 [M+H]⁺. ¹H NMR (400 MHz, methanol-d₄), characteristic peaks, aliphatic integrations are approximate: δ 8.08 (s, 1H), 7.54 (d, J=9.1 Hz, 1H), 7.33 (d, J=8.6 Hz, 2H), 7.29-7.23 (m, 1H), 6.96 (dd, J=9.2, 2.0 Hz, 1H), 6.92-6.85 (m, 3H), 5.23 (s, 2H), 4.42-4.30 (m, 1H), 3.78 (s, 3H), 3.27-3.19 (m, 4H), 2.82-2.63 (m, 6H), 2.53-2.45 (m, 2H), 2.29-2.19 (m, 2H), 2.09-1.89 (m, 4H).

Step 2. Synthesis of 2,3,5-trifluoro-4-hydroxy-N-{[(1r,4r)-4-(6-{4-[4-(piperidin-4-yl)butyl]piperazin-1-yl}-2H-indazol-2-yl)cyclohexyl]methyl}benzamide, hydrochloride salt (C179): A solution of hydrogen chloride in 1.4-dioxane (4 M; 2 mL) was added to a solution of C178 (220 mg, 0.260 mmol) in dichloromethane (8 mL). After the reaction mixture had been stirred at 20° C. for 2 hours, it was concentrated in vacuo to provide C179. Yield: 132 mg, 0.199 mmol, 77%. LCMS m/z 627.4 [M+H]⁺.

Step 3. Synthesis of N-{[(1r,4r)-4-{6-[4-(4-{1-[3-(2,4-dioxo-1,3-diazinan-1-yl)-4-methylbenzoyl]piperidin-4-yl}butyl)piperazin-1-yl]-2H-indazol-2-yl}cyclohexyl]methyl}-2,3,5-trifluoro-4-hydroxybenzamide (20): To a solution of P239 (40 mg, 97 μmol) and C179 (60.5 mg, 91.2 μmol) in dichloromethane (10 mL) was added triethylamine (29 mg, 0.29 mmol), whereupon the reaction mixture was stirred at 20° C. for 2 hours. It was then concentrated in vacuo and purified via reversed-phase HPLC (Column: Waters XBridge C18, 19×150 mm, 5 μm; Mobile phase A: water containing 0.05% formic acid; Mobile phase B: acetonitrile; Gradient: 22% to 52% B; Flow rate: 20 mL/minute) to afford N-{[(1r,4r)-4-{6-[4-(4-{1-[3-(2,4-dioxo-1,3-diazinan-1-yl)-4-methylbenzoyl]piperidin-4-yl}butyl)piperazin-1-yl]-2H-indazol-2-yl}cyclohexyl]methyl}-2,3,5-trifluoro-4-hydroxybenzamide (20) as a solid. Yield: 20.4 mg, 23.8 μmol, 26%. LCMS m/z 857.4 [M+H]⁺. ¹H NMR (400 MHz, DMSO-d₆), characteristic peaks, aliphatic integrations are approximate: δ 10.37 (s, 1H), 8.18 (br s, 1H), 8.17 (s, 1H), 7.87-7.75 (m, 1H), 7.48 (d, J=9.1 Hz, 1H), 7.30 (br s, 1H), 7.28 (AB quartet, upfield doublet is broadened, J_{AB}=7.8 Hz, Δv_{AB}=39.7 Hz, 2H), 7.16-7.08 (m, 1H), 6.89 (d, J=9.2 Hz, 1H), 6.77 (s, 1H), 4.53-4.39 (m, 1H), 4.39-4.27 (m, 1H), 3.87-3.74 (m, 1H), 3.15 (t, J=6.4 Hz, 2H), 3.12-3.05 (m, 4H), 2.83-2.63 (m, 3H), 2.33 (t, J=7.3 Hz, 2H), 2.21 (s, 3H), 2.15-2.05 (m, 2H), 1.93-1.78 (m, 4H), 1.70-1.56 (m, 2H), 1.55-1.40 (m, 3H), 1.12-0.99 (m, 2H).

### Example 21

### N-{[(1r,4r)-4-{6-[6-(3-{1-[(3RS)-2,6-Dioxopiperidin-3-yl]-3-methyl-2-oxo-2,3-dihydro-1H-benzimidazol-5-yl}propyl)-5,6,7,8-tetrahydro-1,6-naphthyridin-2-yl]-2H-indazol-2-yl}cyclohexyl]methyl}-2,3,5-trifluoro-4-hydroxybenzamide (21)

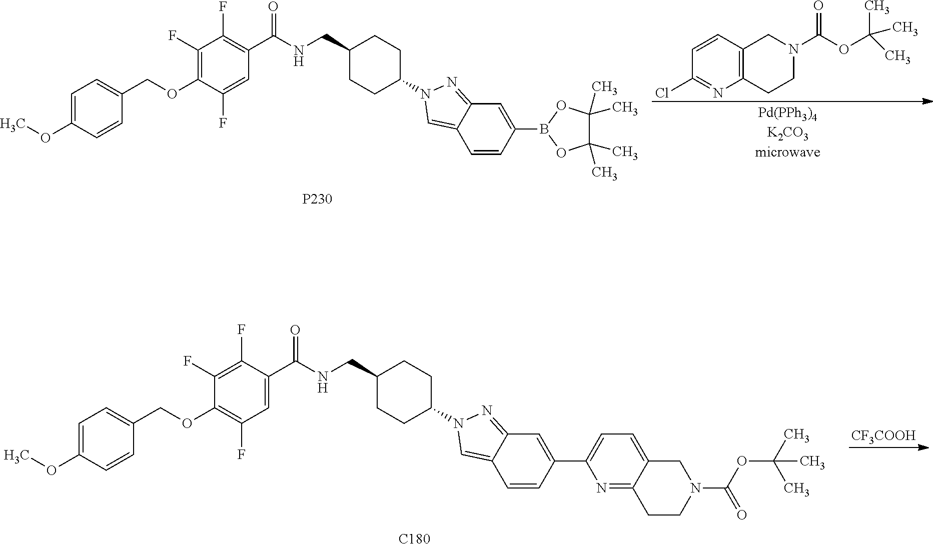

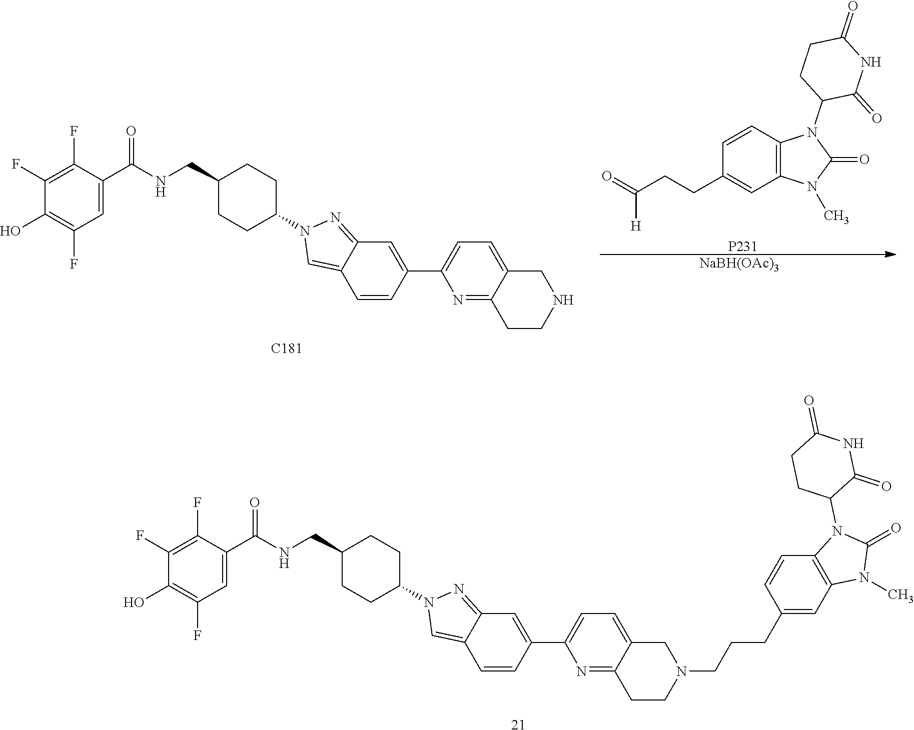

Step 1. Synthesis of tert-butyl 2-{2-[(1r,4r)-4-({2,3,5-trifluoro-4-[(4-methoxyphenyl)methoxy]benzamido}methyl)cyclohexyl]-2H-indazol-6-yl}-7,8-dihydro-1,6-naphthyridine-6(5H)-carboxylate (C180): A mixture of P230 (100 mg, 0.154 mmol), tert-butyl 2-chloro-7,8-dihydro-1,6-naphthyridine-6(5H)-carboxylate (50 mg, 0.19 mmol), tetrakis(triphenylphosphine)palladium(0) (35.6 mg, 30.8 μmol), and potassium carbonate (42.6 mg, 0.308 mmol) in a mixture of N,N-dimethylformamide (2 mL) and water (0.2 mL) was stirred at 120° C. for 20 hours under microwave irradiation. After the reaction mixture had been diluted with water (20 mL), it was extracted with ethyl acetate (3×20 mL), and the combined organic layers were washed with saturated aqueous sodium chloride solution (100 mL), dried over sodium sulfate, filtered, and concentrated in vacuo. Chromatography on silica gel (Gradient: 0% to 15% methanol in dichloromethane) afforded C180 as a light-yellow solid (80 mg). Most of this material was progressed to the following step. LCMS m/z 756.3 [M+H]⁺.

Step 2. Synthesis of 2,3,5-trifluoro-4-hydroxy-N-({(1r,4r)-4-[6-(5,6,7,8-tetrahydro-1,6-naphthyridin-2-yl)-2H-indazol-2-yl]cyclohexyl}methyl)benzamide (C181): Trifluoroacetic acid (0.25 mL, 3.2 mmol) was added to a solution of C180 (from the previous step; 70 mg, 50.13 mmol) in dichloromethane (5 mL), whereupon the reaction mixture was stirred at 25° C. for 2 hours. Concentration in vacuo, followed by silica gel chromatography (Gradient: 0% to 15% methanol in dichloromethane), provided C181 as a light-yellow gum. Yield: 20 mg, 37 μmol, 28% over 2 steps. LCMS m/z 536.3 [M+H]⁺.

Step 3. Synthesis of N-{[(1r,4r)-4-{6-[6-(3-{1-[(3RS)-2,6-dioxopiperidin-3-yl]-3-methyl-2-oxo-2,3-dihydro-1H-benzimidazol-5-yl}propyl)-5,6,7,8-tetrahydro-1,6-naphthyridin-2-yl]-2H-indazol-2-yl}cyclohexyl]methyl}-2,3,5-trifluoro-4-hydroxybenzamide (21): Sodium triacetoxyborohydride (39.6 mg, 0.187 mmol) was added to a mixture of C181 (20 mg, 37 μmol) and P231 (11.8 mg, 37.4 μmol) in dimethyl sulfoxide (2 mL), and the reaction mixture was stirred at 25° C. for 2 hours. It was then diluted with water (20 mL) and extracted with ethyl acetate (3×20 mL); the combined organic layers were washed with saturated aqueous sodium chloride solution (100 mL), dried over sodium sulfate, filtered, and concentrated in vacuo. Silica gel chromatography (Gradient: 0% to 15% methanol in dichloromethane) afforded N-{[(1r,4r)-4-{6-[6-(3-{1-[(3RS)-2,6-dioxopiperidin-3-yl]-3-methyl-2-oxo-2,3-dihydro-1H-benzimidazol-5-yl}propyl)-5,6,7,8-tetrahydro-1,6-naphthyridin-2-yl]-2H-indazol-2-yl}cyclohexyl]methyl}-2,3,5-trifluoro-4-hydroxybenzamide (21) as an off-white solid. Yield: 10.8 mg, 12.9 μmol, 35%. LCMS m/z 835.4 [M+H]⁺. ¹H NMR (400 MHz, DMSO-d₆), characteristic peaks, aliphatic integrations are approximate: δ 11.09 (s, 1H), 8.42 (s, 1H), 8.32-8.26 (m, 1H), 8.25 (br s, 1H), 7.82 (d, J=8.2 Hz, 1H), 7.80 (dd, component of ABX system, J=8.8, 1.4 Hz, 1H), 7.74 (d, half of AB quartet, J=8.8 Hz, 1H), 7.57 (d, J=8.2 Hz, 1H), 7.29 (ddd, J=11.2, 6.4, 2.3 Hz, 1H), 7.09 (br s, 1H), 7.02 (d, half of AB quartet, J=8.0 Hz, 1H), 6.92 (dd, component of ABX system, J=8.1, 1.5 Hz, 1H), 5.34 (dd, J=12.8, 5.4 Hz, 1H), 4.56-4.42 (m, 1H), 3.70 (s, 2H), 3.18 (t, J=6.3 Hz, 2H), 3.05-2.97 (m, 2H), 2.96-2.83 (m, 3H), 2.77-2.55 (m, 6H), 2.23-2.13 (m, 2H), 2.05-1.84 (m, 7H), 1.74-1.61 (m, 1H).

### Example 22

### N-{[(1r,4r)-4-{6-[(3aR,7aS)-2-(3-{1-[(3RS)-2,6-Dioxopiperidin-3-yl]-3-methyl-2-oxo-2,3-dihydro-1H-benzimidazol- 5-yl}propyl)octahydro-5H-pyrrolo[3,4-c]pyridin-5-yl]-2H-indazol-2-yl}cyclohexyl]methyl}-2,3,5-trifluoro-4-hydroxybenzamide, formate salt (22)

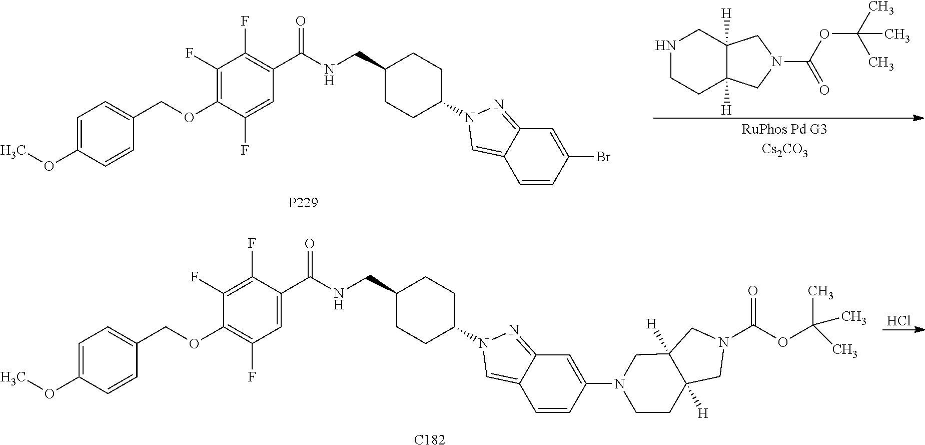 [SMILES: "C182" ; "P229" ; COc1ccc(COc2c(F)cc(C(=O)NC[C@H]3CC[C@H](n4cc5ccc(N6CC[C@@H]7CN(C(=O)OC(C)(C)C)C[C@@H]7C6)cc5n4)CC3)c(F)c2F)cc1 ; COc1ccc(COc2c(F)cc(C(=O)NC[C@H]3CC[C@H](n4cc5ccc(Br)cc5n4)CC3)c(F)c2F)cc1 ; CC(C)(C)OC(=O)N1C[C@H]2CCNC[C@H]2C1 ; Cl]

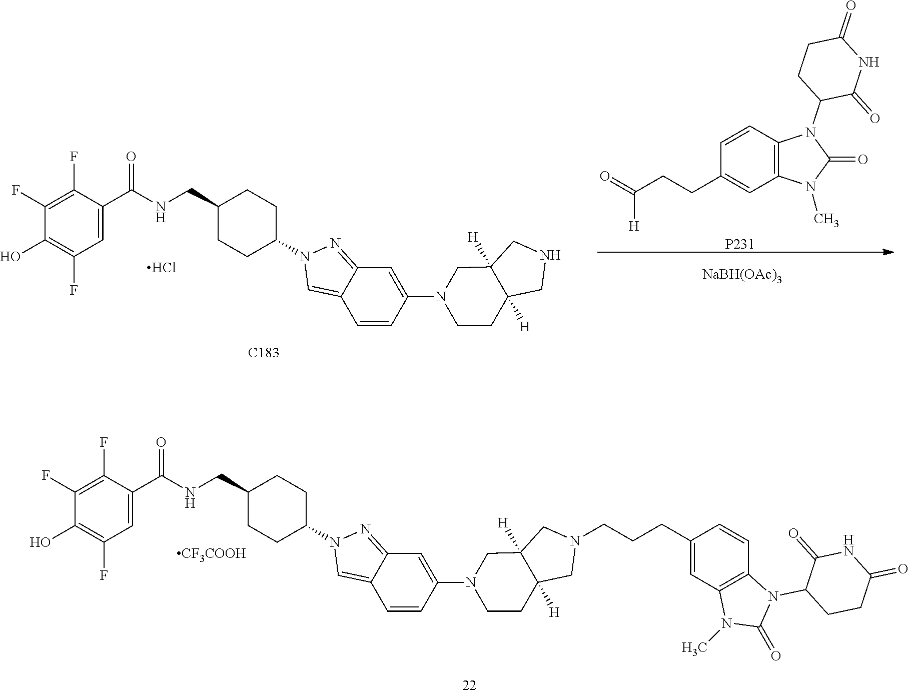

Step 1. Synthesis of tert-butyl (3aS,7aS)-5-{2-[(1r,4r)-4-({2,3,5-trifluoro-4-[(4-methoxyphenyl)methoxy]benzamido}methyl)cyclohexyl]-2H-indazol-6-yl}octahydro-2H-pyrrolo[3,4-c]pyridine-2-carboxylate (C182): To a solution of P229 (170 mg, 0.282 mmol) and tert-butyl (3aS,7aS)-octahydro-2H-pyrrolo[3,4-c]pyridine-2-carboxylate (63.9 mg, 0.282 mmol) in 1,4-dioxane (5 mL) were added (2-dicyclohexylphosphino-2′,6′-diisopropoxy-1,1′-biphenyl)[2-(2′-amino-1,1′-biphenyl)]palladium(II) methanesulfonate (RuPhos Pd G3; 35.4 mg, 42.3 μmol) and cesium carbonate (184 mg, 0.565 mmol). After the reaction mixture had been stirred at 90° C. for 16 hours, LCMS analysis indicated conversion to C182: LCMS m/z 748.4 [M+H]⁺. The reaction mixture was filtered through a pad of diatomaceous earth, and the pad was rinsed with dichloromethane; the combined filtrates were concentrated in vacuo and purified using silica gel chromatography (Gradient: 0% to 50% ethyl acetate in petroleum ether) to afford C182 as a pale-yellow solid. Yield: 110 mg, 0.147 mmol, 52%.

Step 2. Synthesis of 2,3,5-trifluoro-4-hydroxy-N-{[(1r,4r)-4-{6-[(3aR,7aS)-octahydro-5H-pyrrolo[3,4-c]pyridin-5-yl]-2H-indazol-2-yl}cyclohexyl]methyl}benzamide, hydrochloride salt (C183): To a solution of C182 (110 mg, 0.147 mmol) in dichloromethane (2 mL) was added a solution of hydrogen chloride in 1,4-dioxane (4 M; 1 mL, 4 mmol). After the reaction mixture had been stirred at 20° C. for 16 hours, LCMS analysis indicated conversion to C183: LCMS m/z 528.3 [M+H]⁺. Concentration in vacuo provided C183 as a brown solid. Yield: 70 mg, 0.12 mmol, 82%.

Step 3. Synthesis of N-{[(1r,4r)-4-{6-[(3aR,7aS)-2-(3-{1-[(3RS)-2,6-dioxopiperidin-3-yl]-3-methyl-2-oxo-2,3-dihydro-1H-benzimidazol-5-yl}propyl)octahydro-5H-pyrrolo[3,4-c]pyridin-5-yl]-2H-indazol-2-yl}cyclohexyl]methyl}-2,3,5-trifluoro-4-hydroxybenzamide, formate salt (22): Sodium triacetoxyborohydride (112 mg, 0.528 mmol) was added to a solution of C183 (70 mg, 0.12 mmol) and P231 (46.0 mg, 0.146 mmol) in dichloromethane (2 mL), whereupon the reaction mixture was stirred at 20° C. for 16 hours. It was then concentrated in vacuo and purified via reversed-phase HPLC (Column: Waters XBridge C18, 19×150 mm, 5 μm; Mobile phase A: water containing 0.1% formic acid; Mobile phase B: acetonitrile; Gradient: 20% to 50% B; Flow rate: 20 mL/minute) to afford N-{[(1r,4r)-4-{6-[(3aR,7aS)-2-(3-{1-[(3RS)-2,6-dioxopiperidin-3-yl]-3-methyl-2-oxo-2,3-dihydro-1H-benzimidazol-5-yl}propyl)octahydro-5H-pyrrolo[3,4-c]pyridin-5-yl]-2H-indazol-2-yl}cyclohexyl]methyl}-2,3,5-trifluoro-4-hydroxybenzamide, formate salt (22) as a yellow solid and oil. Yield: 21.2 mg, 24.3 μmol, 20%. LCMS m/z 827.4 [M+H]⁺. ¹H NMR (400 MHz, DMSO-d₆), characteristic peaks, aliphatic integrations are approximate: δ 11.08 (s, 1H), 8.25 (s, 1H), 8.13 (s, 1H), 7.69-7.60 (m, 1H), 7.46 (d, J=9.1 Hz, 1H), 7.09-7.01 (m, 1H), 7.03 (br s, 1H), 6.93 (AB quartet, upfield doublet is broadened, J_{AB}=8.0 Hz, Δv_{AB}=51.7 Hz, 2H), 6.82 (dd, J=9.2, 2.0 Hz, 1H), 6.67 (br s, 1H), 5.33 (dd, J=12.7, 5.4 Hz, 1H), 4.37-4.24 (m, 1H), 3.31 (s, 3H), 3.29-3.22 (m, 3H), 3.22-3.09 (m, 3H), 3.09-3.00 (m, 1H), 2.95-2.82 (m, 3H), 2.76-2.55 (m, 6H), 2.36-2.25 (m, 1H), 2.15-2.04 (m, 2H), 2.04-1.94 (m, 1H), 1.93-1.67 (m, 7H), 1.67-1.56 (m, 1H), 1.24-1.09 (m, 2H).

### Protein Degrader Compounds

Table 3 shows additional protein degrader compounds of the disclosure of Formula II. In the IUPAC names for these Examples, the stereochemistry of the 2,6-dioxopiperidine moiety has been indicated as (3RS), to emphasize that these compounds are racemic at that 3-position. Other stereocenters that are drawn with a straight bond should also be understood as representing an equal mixture of both possible stereochemistries at that center.

TABLE 3

| Example | Structure | IUPAC Name |
|---|---|---|
| 23 | 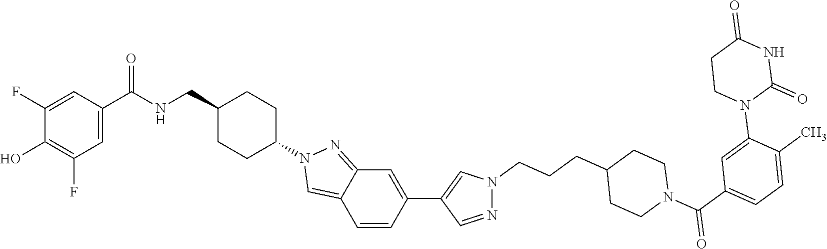 [SMILES: Cc1ccc(C(=O)N2CCC(CCCn3cc(-c4ccc5cn([C@H]6CC[C@H](CNC(=O)c7cc(F)c(O)c(F)c7)CC6)nc5c4)cn3)CC2)cc1N1CCC(=O)NC1=O] | N-{[(1r,4r)-4-{6-[1-(3-{1-[3-(2,4- dioxo-1,3-diazinan-1-yl)-4- methylbenzoyl]piperidin-4- yl}propyl)-1H-pyrazol-4-yl]-2H- indazol-2-yl}cyclohexyl]methyl}- 3,5-difluoro-4-hydroxybenzamide |
| 24 | 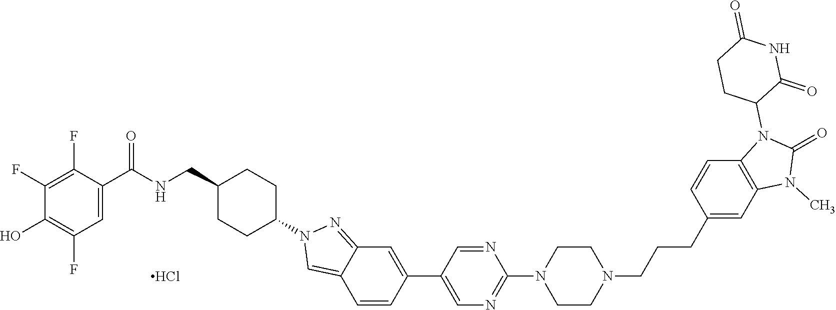 [SMILES: Cn1c(=O)n(C2CCC(=O)NC2=O)c2ccc(CCCN3CCN(c4ncc(-c5ccc6cn([C@H]7CC[C@H](CNC(=O)c8cc(F)c(O)c(F)c8F)CC7)nc6c5)cn4)CC3)cc21 ; Cl] | N-{[(1r,4r)-4-(6-{2-[4-(3-{1-[(3RS)- 2,6-dioxopiperidin-3-yl]-3-methyl- 2-oxo-2,3-dihydro-1H- benzimidazol-5- yl}propyl)piperazin-1-yl]pyrimidin- 5-yl}-2H-indazol-2- yl)cyclohexyl]methyl}-2,3,5- trifluoro-4-hydroxybenzamide, hydrochloride salt |
| 25 | 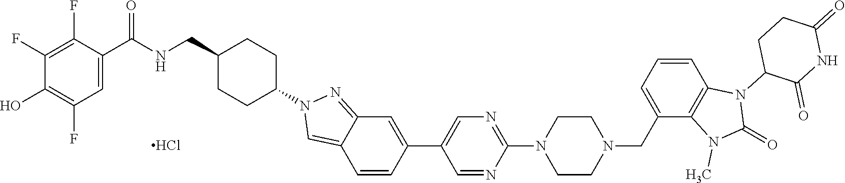 [SMILES: Cn1c(=O)n(C2CCC(=O)NC2=O)c2cccc(CN3CCN(c4ncc(-c5ccc6cn([C@H]7CC[C@H](CNC(=O)c8cc(F)c(O)c(F)c8F)CC7)nc6c5)cn4)CC3)c21 ; Cl] | N-{[(1r,4r)-4-(6-{2-[4-({1-[(3RS)- 2,6-dioxopiperidin-3-yl]-3-methyl- 2-oxo-2,3-dihydro-1H- benzimidazol-4- yl}methyl)piperazin-1-yl]pyrimidin- 5-yl}-2H-indazol-2- yl)cyclohexyl]methyl}-2,3,5- trifluoro-4-hydroxybenzamide, hydrochloride salt |
| 26 | 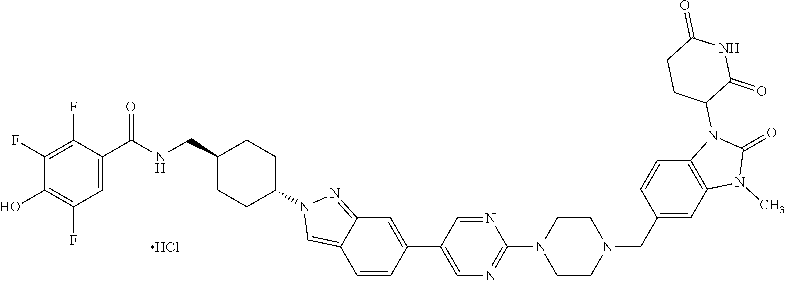 [SMILES: Cn1c(=O)n(C2CCC(=O)NC2=O)c2ccc(CN3CCN(c4ncc(-c5ccc6cn([C@H]7CC[C@H](CNC(=O)c8cc(F)c(O)c(F)c8F)CC7)nc6c5)cn4)CC3)cc21 ; Cl] | N-{[(1r,4r)-4-(6-{2-[4-({1-[(3RS)- 2,6-dioxopiperidin-3-yl]-3-methyl- 2-oxo-2,3-dihydro-1H- benzimidazol-5- yl}methyl)piperazin-1-yl]pyrimidin- 5-yl}-2H-indazol-2- yl)cyclohexyl]methyl}-2,3,5- trifluoro-4-hydroxybenzamide, hydrochloride salt |
| 27 | 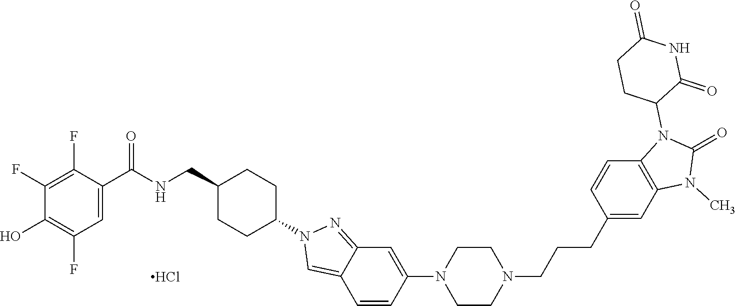 [SMILES: Cn1c(=O)n(C2CCC(=O)NC2=O)c2ccc(CCCN3CCN(c4ccc5cn([C@H]6CC[C@H](CNC(=O)c7cc(F)c(O)c(F)c7F)CC6)nc5c4)CC3)cc21 ; Cl] | N-{[(1r,4r)-4-{6-[4-(3-{1-[(3RS)- 2,6-dioxopiperidin-3-yl]-3-methyl- 2-oxo-2,3-dihydro-1H- benzimidazol-5- yl}propyl)piperazin-1-yl]-2H- indazol-2-yl}cyclohexyl]methyl}- 2,3,5-trifluoro-4- hydroxybenzamide, hydrochloride salt |
| 28 | 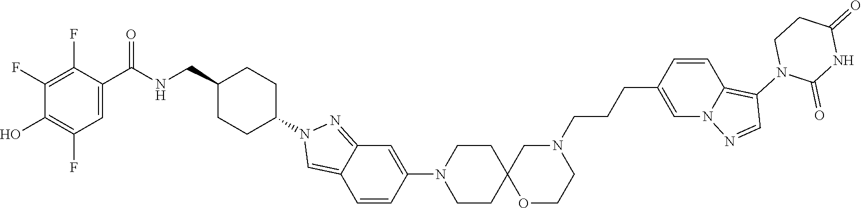 [SMILES: O=C1CCN(c2cnn3cc(CCCN4CCOC5(CCN(c6ccc7cn([C@H]8CC[C@H](CNC(=O)c9cc(F)c(O)c(F)c9F)CC8)nc7c6)CC5)C4)ccc23)C(=O)N1] | N-({(1r,4r)-4-[6-(4-{3-[3-(2,4-dioxo- 1,3-diazinan-1-yl)pyrazolo[1,5- a]pyridin-6-yl]propyl}-1-oxa-4,9- diazaspiro[5.5]undecan-9-yl)-2H- indazol-2-yl]cyclohexyl}methyl)- 2,3,5-trifluoro-4- hydroxybenzamide |
| 29 | 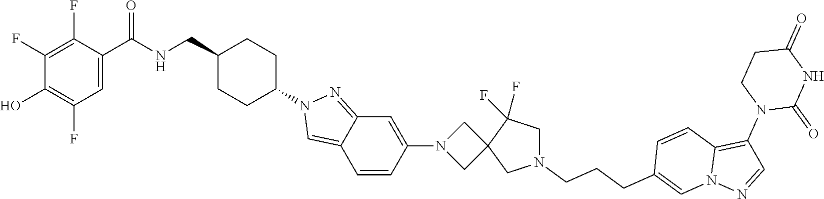 [SMILES: O=C1CCN(c2cnn3cc(CCCN4CC(F)(F)C5(C4)CN(c4ccc6cn([C@H]7CC[C@H](CNC(=O)c8cc(F)c(O)c(F)c8F)CC7)nc6c4)C5)ccc23)C(=O)N1] | N-({(1r,4r)-4-[6-(6-{3-[3-(2,4-dioxo- 1,3-diazinan-1-yl)pyrazolo[1,5- a]pyridin-6-yl]propyl}-8,8-difluoro- 2,6-diazaspiro[3.4]octan-2-yl)-2H- indazol-2-yl]cyclohexyl}methyl)- 2,3,5-trifluoro-4- hydroxybenzamide |
| 30 | 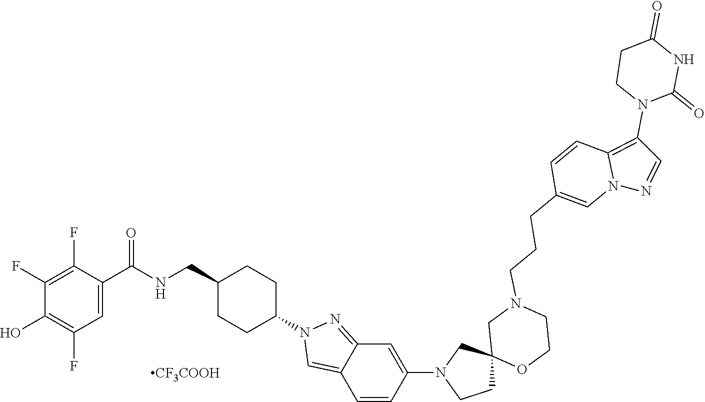 [SMILES: O=C1CCN(c2cnn3cc(CCCN4CCO[C@]5(CCN(c6ccc7cn([C@H]8CC[C@H](CNC(=O)c9cc(F)c(O)c(F)c9F)CC8)nc7c6)C5)C4)ccc23)C(=O)N1 ; "·CF3COOH"] | N-{[(1r,4r)-4-{6-[(5R)-9-{3-[3-(2,4- dioxo-1,3-diazinan-1- yl)pyrazolo[1,5-a]pyridin-6- yl]propyl}-6-oxa-2,9- diazaspiro[4.5]decan-2-yl]-2H- indazol-2-yl}cyclohexyl]methyl}- 2,3,5-trifluoro-4- hydroxybenzamide, trifluoroacetate salt |
| | or | or |
| | 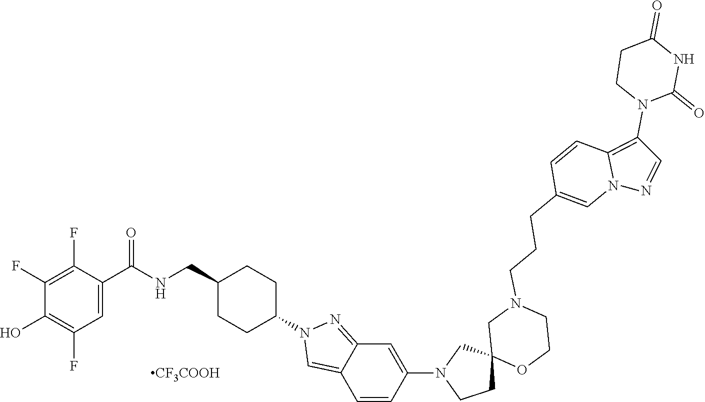 [SMILES: O=C1CCN(c2cnn3cc(CCCN4CCO[C@@]5(CCN(c6ccc7cn([C@H]8CC[C@H](CNC(=O)c9cc(F)c(O)c(F)c9F)CC8)nc7c6)C5)C4)ccc23)C(=O)N1 ; "·CF3COOH"] | N-{[(1r,4r)-4-{6-[(5S)-9-{3-[3-(2,4- dioxo-1,3-diazinan-1- yl)pyrazolo[1,5-a]pyridin-6- yl]propyl}-6-oxa-2,9- diazaspiro[4.5]decan-2-yl]-2H- indazol-2-yl}cyclohexyl]methyl}- 2,3,5-trifluoro-4- hydroxybenzamide, trifluoroacetate salt (See footnote 5 in Table 4) |
| 31 | 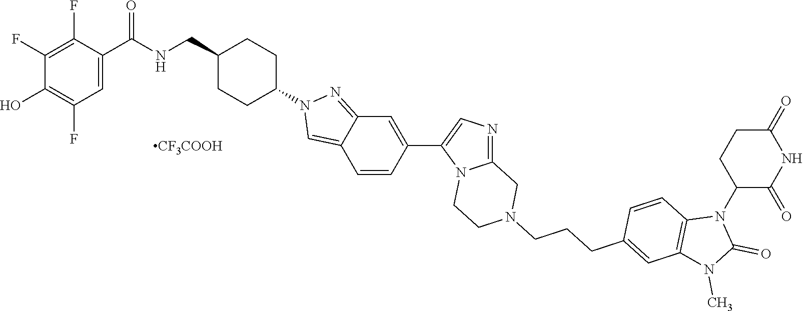 [SMILES: Cn1c(=O)n(C2CCC(=O)NC2=O)c2ccc(CCCN3CCn4c(-c5ccc6cn([C@H]7CC[C@H](CNC(=O)c8cc(F)c(O)c(F)c8F)CC7)nc6c5)cnc4C3)cc21 ; "·CF3COOH"] | N-{[(1r,4r)-4-{6-[7-(3-{1-[(3RS)- 2,6-dioxopiperidin-3-yl]-3-methyl- 2-oxo-2,3-dihydro-1H- benzimidazol-5-yl}propyl)-5,6,7,8- tetrahydroimidazo[1,2-a]pyrazin-3- yl]-2H-indazol-2- yl}cyclohexyl]methyl}-2,3,5- trifluoro-4-hydroxybenzamide, trifluoroacetate salt |
| 32 | 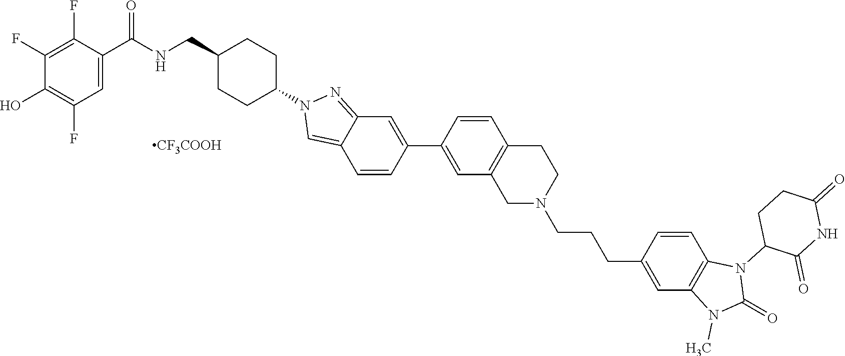 [SMILES: Cn1c(=O)n(C2CCC(=O)NC2=O)c2ccc(CCCN3CCc4ccc(-c5ccc6cn([C@H]7CC[C@H](CNC(=O)c8cc(F)c(O)c(F)c8F)CC7)nc6c5)cc4C3)cc21 ; "·CF3COOH"] | N-{[(1r,4r)-4-{6-[2-(3-{1-[(3RS)- 2,6-dioxopiperidin-3-yl]-3-methyl- 2-oxo-2,3-dihydro-1H- benzimidazol-5-yl}propyl)-1,2,3,4- tetrahydroisoquinolin-7-yl]-2H- indazol-2-yl}cyclohexyl]methyl}- 2,3,5-trifluoro-4- hydroxybenzamide, trifluoroacetate salt |
| 33 | 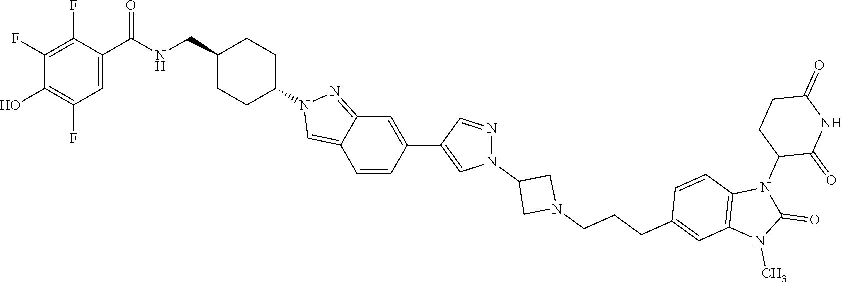 [SMILES: Cn1c(=O)n(C2CCC(=O)NC2=O)c2ccc(CCCN3CC(n4cc(-c5ccc6cn([C@H]7CC[C@H](CNC(=O)c8cc(F)c(O)c(F)c8F)CC7)nc6c5)cn4)C3)cc21] | N-{[(1r,4r)-4-(6-{1-[1-(3-{1-[(3RS)- 2,6-dioxopiperidin-3-yl]-3-methyl- 2-oxo-2,3-dihydro-1H- benzimidazol-5-yl}propyl)azetidin- 3-yl]-1H-pyrazol-4-yl}-2H-indazol- 2-yl)cyclohexyl]methyl}-2,3,5- trifluoro-4-hydroxybenzamide |
| 34 | 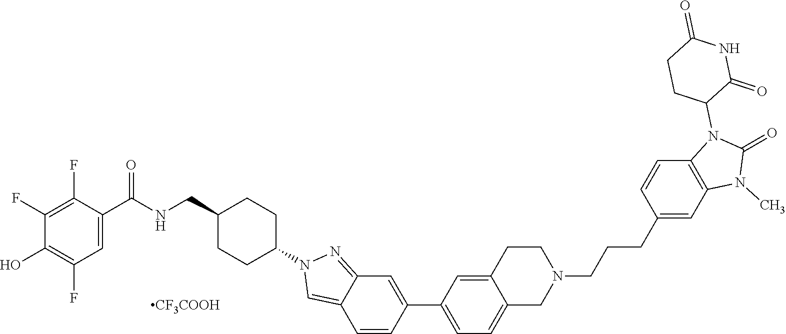 [SMILES: Cn1c(=O)n(C2CCC(=O)NC2=O)c2ccc(CCCN3CCc4cc(-c5ccc6cn([C@H]7CC[C@H](CNC(=O)c8cc(F)c(O)c(F)c8F)CC7)nc6c5)ccc4C3)cc21 ; "·CF3COOH"] | N-{[(1r,4r)-4-{6-[2-(3-{1-[(3RS)- 2,6-dioxopiperidin-3-yl]-3-methyl- 2-oxo-2,3-dihydro-1H- benzimidazol-5-yl}propyl)-1,2,3,4- tetrahydroisoquinolin-6-yl]-2H- indazol-2-yl}cyclohexyl]methyl}- 2,3,5-trifluoro-4- hydroxybenzamide, trifluoroacetate salt |
| 35 | 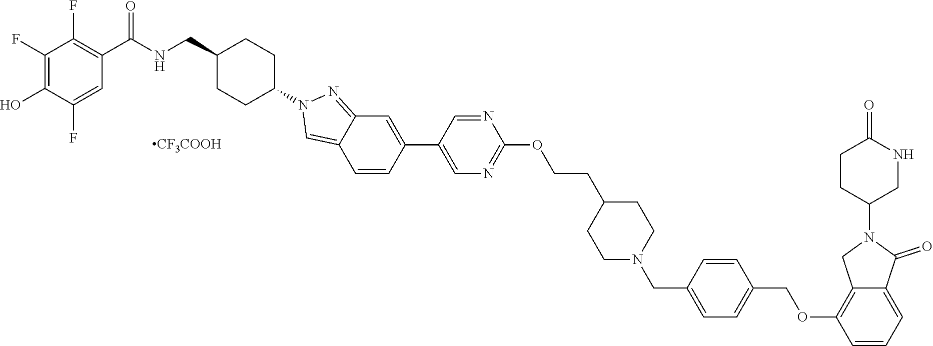 [SMILES: O=C1CCC(N2Cc3c(OCc4ccc(CN5CCC(CCOc6ncc(-c7ccc8cn([C@H]9CC[C@H](CNC(=O)c%10cc(F)c(O)c(F)c%10F)CC9)nc8c7)cn6)CC5)cc4)cccc3C2=O)CN1 ; "·CF3COOH"] | N-({(1r,4r)-4-[6-(2-{2-[1-({4-[{{2- [(3RS)-2,6-dioxopiperidin-3-yl]-1- oxo-2,3-dihydro-1H-isoindol-4- yl}oxy)methyl]phenyl}methyl)piperi- din-4-yl]ethoxy}pyrimidin-5-yl)-2H- indazol-2-yl]cyclohexyl}methyl)- 2,3,5-trifluoro-4- hydroxybenzamide, trifluoroacetate salt |
| 36 | 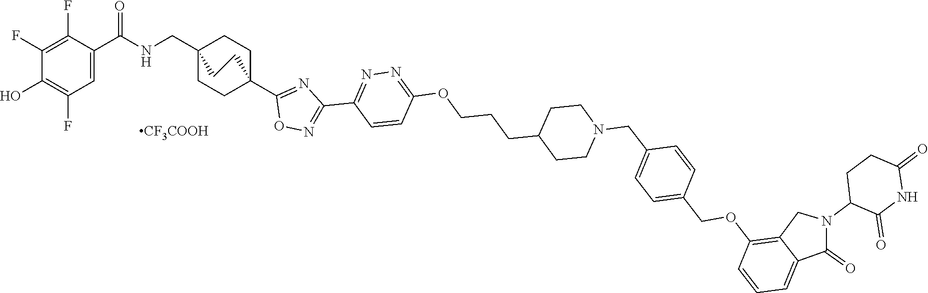 [SMILES: O=C1CCC(N2Cc3c(OCc4ccc(CN5CCC(CCCOc6ccc(-c7noc([C@]89CC[C@](CNC(=O)c%10cc(F)c(O)c(F)c%10F)(CC8)CC9)n7)nn6)CC5)cc4)cccc3C2=O)C(=O)N1 ; "·CF3COOH"] | N-({4-[3-(6-{3-[1-({4-[{{2-[(3RS)- 2,6-dioxopiperidin-3-yl]-1-oxo-2,3- dihydro-1H-isoindol-4- yl}oxy)methyl]phenyl}methyl)piperi- din-4-yl]propoxy}pyridazin-3-yl)- 1,2,4-oxadiazol-5- yl]bicyclo[2.2.2]octan-1-yl}methyl)- 2,3,5-trifluoro-4- hydroxybenzamide, trifluoroacetate salt |
| 37 | 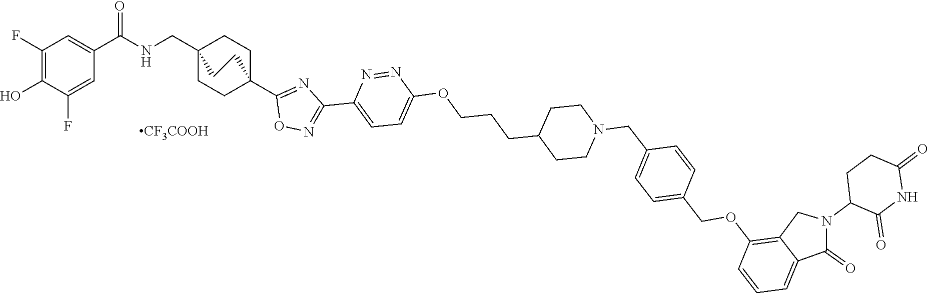 [SMILES: O=C1CCC(N2Cc3c(OCc4ccc(CN5CCC(CCCOc6ccc(-c7noc([C@]89CC[C@](CNC(=O)c%10cc(F)c(O)c(F)c%10)(CC8)CC9)n7)nn6)CC5)cc4)cccc3C2=O)C(=O)N1 ; "·CF3COOH"] | N-({4-[3-(6-{3-[1-({4-[({2-[(3RS)- 2,6-dioxopiperidin-3-yl]-1-oxo-2,3- dihydro-1H-isoindol-4- yl}oxy)methyl]phenyl}methyl)piperi- din-4-yl]propoxy}pyridazin-3-yl)- 1,2,4-oxadiazol-5- yl]bicyclo[2.2.2]octan-1-yl}methyl)- 3,5-difluoro-4-hydroxybenzamide, trifluoroacetate salt |
| 38 | 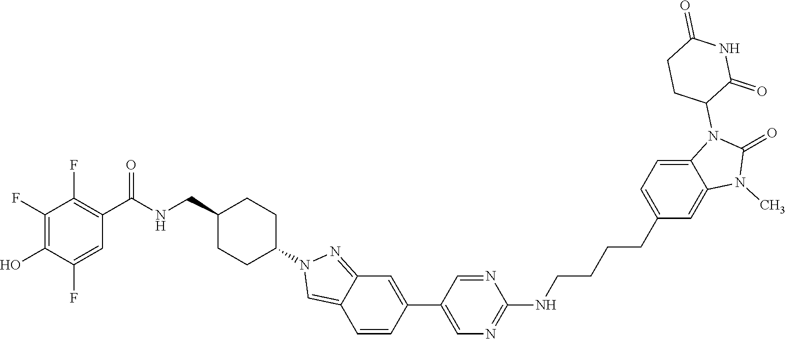 [SMILES: Cn1c(=O)n(C2CCC(=O)NC2=O)c2ccc(CCCCNc3ncc(-c4ccc5cn([C@H]6CC[C@H](CNC(=O)c7cc(F)c(O)c(F)c7F)CC6)nc5c4)cn3)cc21] | N-{[(1r,4r)-4-(6-{2-[(4-{1-[(3RS)- 2,6-dioxopiperidin-3-yl]-3-methyl- 2-oxo-2,3-dihydro-1H- benzimidazol-5- yl}butyl)amino]pyrimidin-5-yl}-2H- indazol-2-yl)cyclohexyl]methyl}- 2,3,5-trifluoro-4- hydroxybenzamide |
| 39 | 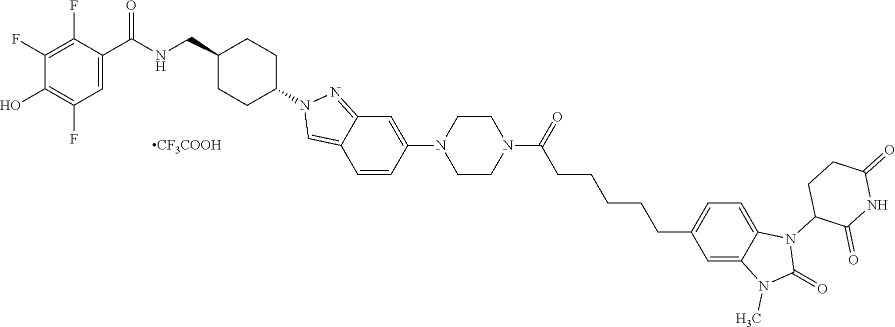 [SMILES: Cn1c(=O)n(C2CCC(=O)NC2=O)c2ccc(CCCCCC(=O)N3CCN(c4ccc5cn([C@H]6CC[C@H](CNC(=O)c7cc(F)c(O)c(F)c7F)CC6)nc5c4)CC3)cc21 ; "·CF3COOH"] | N-{[(1r,4r)-4-{6-[4-(6-{1-[(3RS)- 2,6-dioxopiperidin-3-yl]-3-methyl- 2-oxo-2,3-dihydro-1H- benzimidazol-5- yl}hexanoyl)piperazin-1-yl]-2H- indazol-2-yl}cyclohexyl]methyl}- 2,3,5-trifluoro-4- hydroxybenzamide, trifluoroacetate salt |
| 40 | 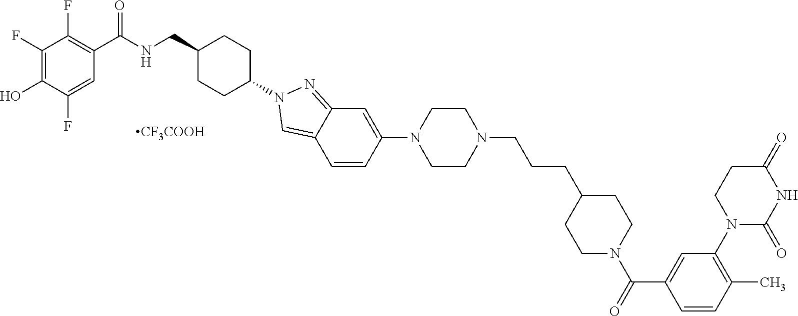 [SMILES: Cc1ccc(C(=O)N2CCC(CCCN3CCN(c4ccc5cn([C@H]6CC[C@H](CNC(=O)c7cc(F)c(O)c(F)c7F)CC6)nc5c4)CC3)CC2)cc1N1CCC(=O)NC1=O ; "·CF3COOH"] | N-{[(1r,4r)-4-{6-[4-(3-{1-[3-(2,4- dioxo-1,3-diazinan-1-yl)-4- methylbenzoyl]piperidin-4- yl}propyl)piperazin-1-yl]-2H- indazol-2-yl}cyclohexyl]methyl}- 2,3,5-trifluoro-4- hydroxybenzamide, trifluoroacetate salt |
| 41 | 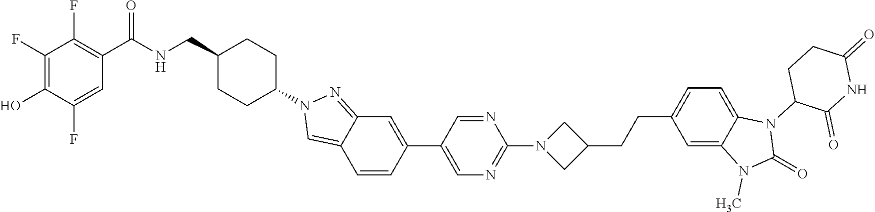 [SMILES: Cn1c(=O)n(C2CCC(=O)NC2=O)c2ccc(CCC3CN(c4ncc(-c5ccc6cn([C@H]7CC[C@H](CNC(=O)c8cc(F)c(O)c(F)c8F)CC7)nc6c5)cn4)C3)cc21] | N-{[(1r,4r)-4-(6-{2-[3-(2-{1-[(3RS)- 2,6-dioxopiperidin-3-yl]-3-methyl- 2-oxo-2,3-dihydro-1H- benzimidazol-5-yl}ethyl)azetidin-1- yl]pyrimidin-5-yl}-2H-indazol-2- yl)cyclohexyl]methyl}-2,3,5- trifluoro-4-hydroxybenzamide |
| 42 | 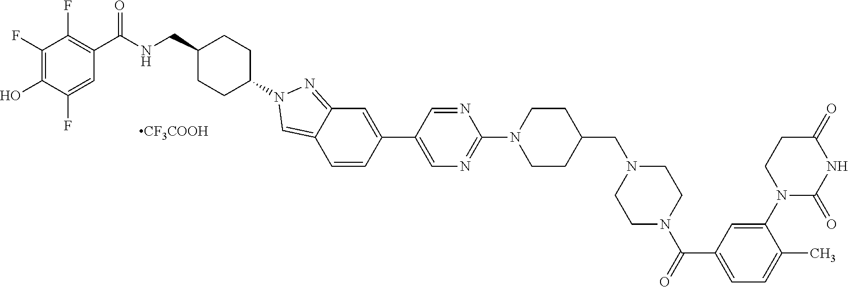 [SMILES: Cc1ccc(C(=O)N2CCN(CC3CCN(c4ncc(-c5ccc6cn([C@H]7CC[C@H](CNC(=O)c8cc(F)c(O)c(F)c8F)CC7)nc6c5)cn4)CC3)CC2)cc1N1CCC(=O)NC1=O ; "·CF3COOH"] | N-{[(1r,4r)-4-(6-{2-[4-({4-[3-(2,4- dioxo-1,3-diazinan-1-yl)-4- methylbenzoyl]piperazin-1- yl}methyl)piperidin-1-yl]pyrimidin- 5-yl}-2H-indazol-2- yl)cyclohexyl]methyl}-2,3,5- trifluoro-4-hydroxybenzamide, trifluoroacetate salt |
| 43 | 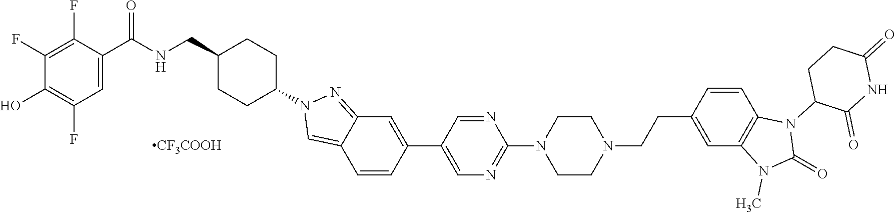 [SMILES: Cn1c(=O)n(C2CCC(=O)NC2=O)c2ccc(CCN3CCN(c4ncc(-c5ccc6cn([C@H]7CC[C@H](CNC(=O)c8cc(F)c(O)c(F)c8F)CC7)nc6c5)cn4)CC3)cc21 ; "·CF3COOH"] | N-{[(1r,4r)-4-(6-{2-[4-(2-{1-[(3RS)- 2,6-dioxopiperidin-3-yl]-3-methyl- 2-oxo-2,3-dihydro-1H- benzimidazol-5-yl}ethyl)piperazin- 1-yl]pyrimidin-5-yl}-2H-indazol-2- yl)cyclohexyl]methyl}-2,3,5- trifluoro-4-hydroxybenzamide, trifluoroacetate salt |
| 44 | 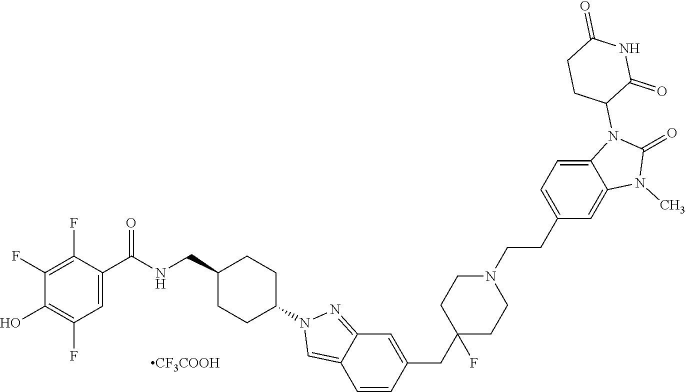 [SMILES: Cn1c(=O)n(C2CCC(=O)NC2=O)c2ccc(CCN3CCC(F)(Cc4ccc5cn([C@H]6CC[C@H](CNC(=O)c7cc(F)c(O)c(F)c7F)CC6)nc5c4)CC3)cc21 ; "·CF3COOH"] | N-{[(1r,4r)-4-(6-{[1-(2-{1-[(3RS)- 2,6-dioxopiperidin-3-yl]-3-methyl- 2-oxo-2,3-dihydro-1H- benzimidazol-5-yl}ethyl)-4- fluoropiperidin-4-yl]methyl}-2H- indazol-2-yl)cyclohexyl]methyl}- 2,3,5-trifluoro-4- hydroxybenzamide, trifluoroacetate salt |
| 45 | 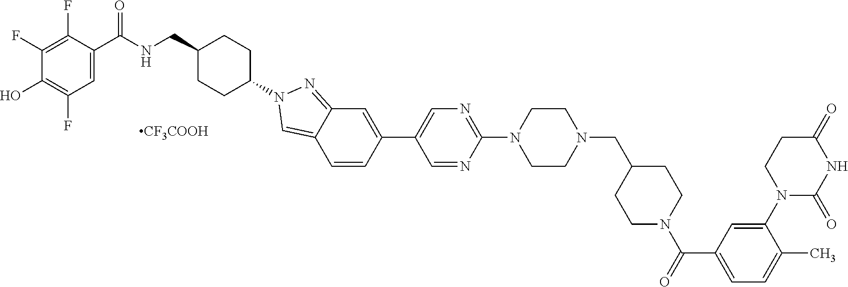 [SMILES: Cc1ccc(C(=O)N2CCC(CN3CCN(c4ncc(-c5ccc6cn([C@H]7CC[C@H](CNC(=O)c8cc(F)c(O)c(F)c8F)CC7)nc6c5)cn4)CC3)CC2)cc1N1CCC(=O)NC1=O ; "·CF3COOH"] | N-{[(1r,4r)-4-(6-{2-[4-({1-[3-(2,4- dioxo-1,3-diazinan-1-yl)-4- methylbenzoyl]piperidin-4- yl}methyl)piperazin-1-yl]pyrimidin- 5-yl}-2H-indazol-2- yl)cyclohexyl]methyl}-2,3,5- trifluoro-4-hydroxybenzamide, trifluoroacetate salt |
| 46 | 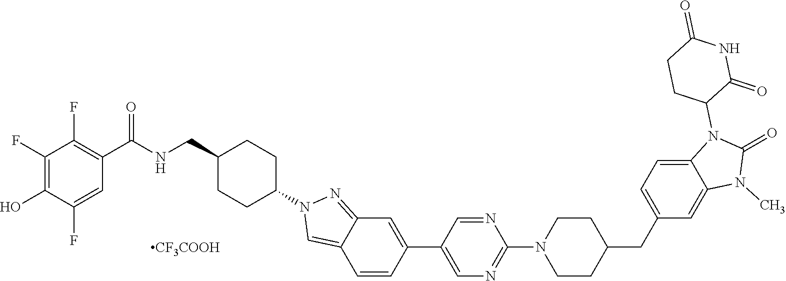 [SMILES: Cn1c(=O)n(C2CCC(=O)NC2=O)c2ccc(CC3CCN(c4ncc(-c5ccc6cn([C@H]7CC[C@H](CNC(=O)c8cc(F)c(O)c(F)c8F)CC7)nc6c5)cn4)CC3)cc21 ; "·CF3COOH"] | N-{[(1r,4r)-4-(6-{2-[4-({1-[(3RS)- 2,6-dioxopiperidin-3-yl]-3-methyl- 2-oxo-2,3-dihydro-1H- benzimidazol-5- yl}methyl)piperidin-1-yl]pyrimidin- 5-yl}-2H-indazol-2- yl)cyclohexyl]methyl}-2,3,5- trifluoro-4-hydroxybenzamide, trifluoroacetate salt |
| 47 | 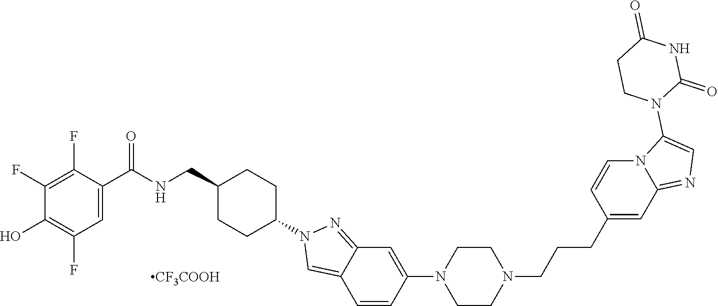 [SMILES: O=C1CCN(c2cnc3cc(CCCN4CCN(c5ccc6cn([C@H]7CC[C@H](CNC(=O)c8cc(F)c(O)c(F)c8F)CC7)nc6c5)CC4)ccn23)C(=O)N1 ; "·CF3COOH"] | N-({(1r,4r)-4-[6-(4-{3-[3-(2,4-dioxo- 1,3-diazinan-1-yl)imidazo[1,2- a]pyridin-7-yl]propyl}piperazin-1- yl)-2H-indazol-2- yl]cyclohexyl}methyl)-2,3,5- trifluoro-4-hydroxybenzamide, trifluoroacetate salt |
| 48 | 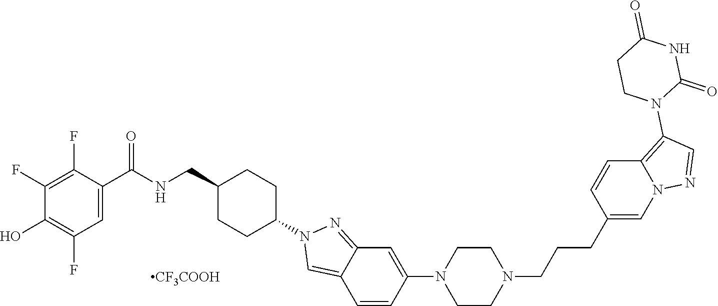 [SMILES: O=C1CCN(c2cnn3cc(CCCN4CCN(c5ccc6cn([C@H]7CC[C@H](CNC(=O)c8cc(F)c(O)c(F)c8F)CC7)nc6c5)CC4)ccc23)C(=O)N1 ; "·CF3COOH"] | N-({(1r,4r)-4-[6-(4-{3-[3-(2,4-dioxo- 1,3-diazinan-1-yl)pyrazolo[1,5- a]pyridin-6-yl]propyl}piperazin-1- yl)-2H-indazol-2- yl]cyclohexyl}methyl)-2,3,5- trifluoro-4-hydroxybenzamide, trifluoroacetate salt |
| 49 | 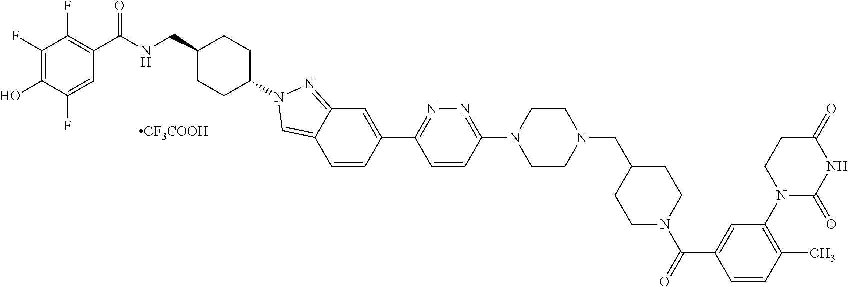 [SMILES: Cc1ccc(C(=O)N2CCC(CN3CCN(c4ccc(-c5ccc6cn([C@H]7CC[C@H](CNC(=O)c8cc(F)c(O)c(F)c8F)CC7)nc6c5)nn4)CC3)CC2)cc1N1CCC(=O)NC1=O ; "·CF3COOH"] | N-{[(1r,4r)-4-(6-{6-[4-({1-[3-(2,4- dioxo-1,3-diazinan-1-yl)-4- methylbenzoyl]piperidin-4- yl}methyl)piperazin-1-yl]pyridazin- 3-yl}-2H-indazol-2- yl)cyclohexyl]methyl}-2,3,5- trifluoro-4-hydroxybenzamide, trifluoroacetate salt |
| 50 | 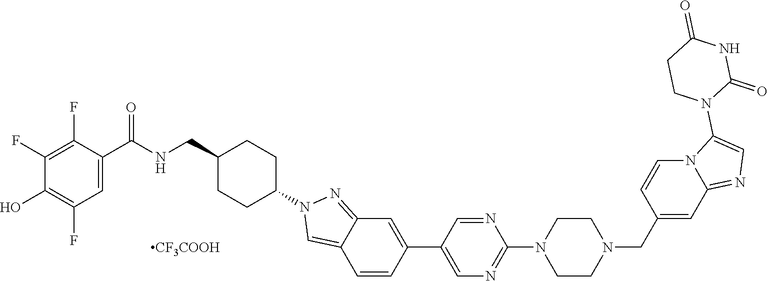 [SMILES: O=C1CCN(c2cnc3cc(CN4CCN(c5ncc(-c6ccc7cn([C@H]8CC[C@H](CNC(=O)c9cc(F)c(O)c(F)c9F)CC8)nc7c6)cn5)CC4)ccn23)C(=O)N1 ; "·CF3COOH"] | N-{[(1r,4r)-4-{6-[2-(4-{[3-(2,4- dioxo-1,3-diazinan-1- yl)imidazo[1,2-a]pyridin-7- yl]methyl}piperazin-1-yl)pyrimidin- 5-yl]-2H-indazol-2- yl}cyclohexyl]methyl}-2,3,5- trifluoro-4-hydroxybenzamide, trifluoroacetate salt |
| 51 | 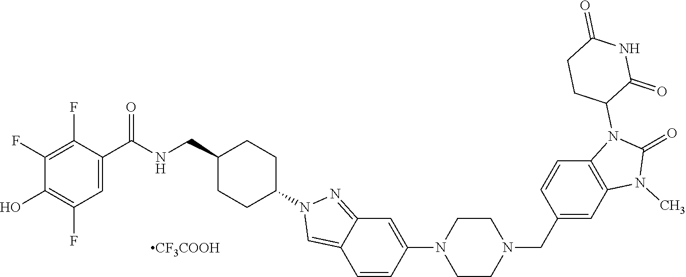 [SMILES: Cn1c(=O)n(C2CCC(=O)NC2=O)c2ccc(CN3CCN(c4ccc5cn([C@H]6CC[C@H](CNC(=O)c7cc(F)c(O)c(F)c7F)CC6)nc5c4)CC3)cc21 ; "·CF3COOH"] | N-{[(1r,4r)-4-{6-[4-({1-[(3RS)-2,6- dioxopiperidin-3-yl]-3-methyl-2- oxo-2,3-dihydro-1H-benzimidazol- 5-yl}methyl)piperazin-1-yl]-1H- indazol-2-yl}cyclohexyl]methyl}- 2,3,5-trifluoro-4- hydroxybenzamide, trifluoroacetate salt |
| 52 | 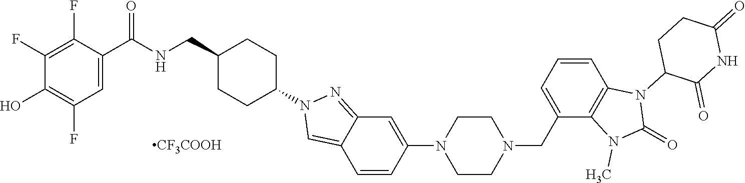 [SMILES: Cn1c(=O)n(C2CCC(=O)NC2=O)c2cccc(CN3CCN(c4ccc5cn([C@H]6CC[C@H](CNC(=O)c7cc(F)c(O)c(F)c7F)CC6)nc5c4)CC3)c21 ; "·CF3COOH"] | N-{[(1r,4r)-4-{6-[4-({1-[(3RS)-2,6- dioxopiperidin-3-yl]-3-methyl-2- oxo-2, 3-dihydro-1H-benzimidazol- 4-yl}methyl)piperazin-1-yl]-2H- indazol-2-yl}cyclohexyl]methyl}- 2,3,5-trifluoro-4- hydroxybenzamide, trifluoroacetate salt |
| 53 | 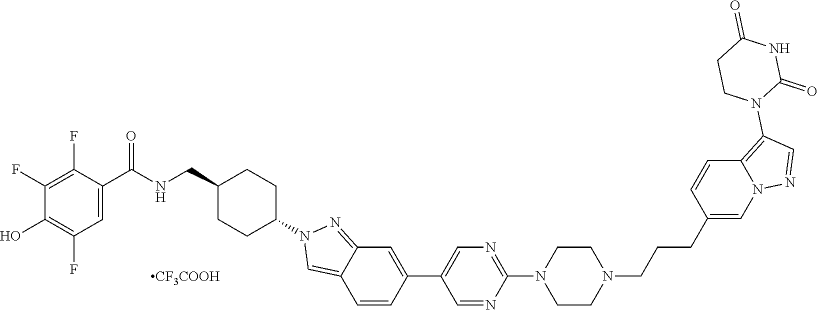 [SMILES: O=C1CCN(c2cnn3cc(CCCN4CCN(c5ncc(-c6ccc7cn([C@H]8CC[C@H](CNC(=O)c9cc(F)c(O)c(F)c9F)CC8)nc7c6)cn5)CC4)ccc23)C(=O)N1 ; "·CF3COOH"] | N-{[(1r,4r)-4-{6-[2-(4-{3-[3-(2,4- dioxo-1,3-diazinan-1- yl)pyrazolo[1,5-a]pyridin-6- yl]propyl}piperazin-1-yl)pyrimidin- 5-yl]-2H-indazol-2- yl}cyclohexyl]methyl}-2,3,5- trifluoro-4-hydroxybenzamide, trifluoroacetate salt |
| 54 | 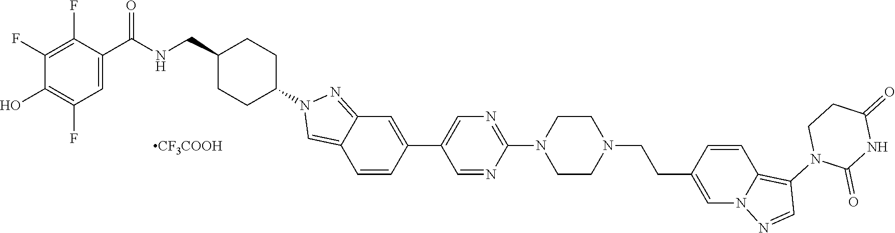 [SMILES: O=C1CCN(c2cnn3cc(CCN4CCN(c5ncc(-c6ccc7cn([C@H]8CC[C@H](CNC(=O)c9cc(F)c(O)c(F)c9F)CC8)nc7c6)cn5)CC4)ccc23)C(=O)N1 ; "·CF3COOH"] | N-{[(1r,4r)-4-{6-[2-(4-{2-[3-(2,4- dioxo-1,3-diazinan-1- yl)pyrazolo[1,5-a]pyridin-6- yl]ethyl}piperazin-1-yl)pyrimidin-5- yl]-2H-indazol-2- yl}cyclohexyl]methyl}-2,3,5- trifluoro-4-hydroxybenzamide, trifluoroacetate salt |
| 55 | 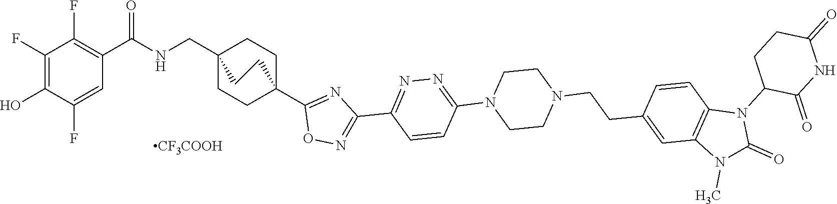 [SMILES: Cn1c(=O)n(C2CCC(=O)NC2=O)c2ccc(CCN3CCN(c4ccc(-c5noc([C@]67CC[C@](CNC(=O)c8cc(F)c(O)c(F)c8F)(CC6)CC7)n5)nn4)CC3)cc21 ; "·CF3COOH"] | N-{[4-(3-{6-[4-(2-{1-[(3RS)-2,6- dioxopiperidin-3-yl]-3-methyl-2- oxo-2,3-dihydro-1H-benzimidazol- 5-yl}ethyl)piperazin-1-yl]pyridazin- 3-yl}-1,2,4-oxadiazol-5- yl)bicyclo[2.2.2]octan-1-yl]methyl}- 2,3,5-trifluoro-4- hydroxybenzamide, trifluoroacetate salt |
| 56 | 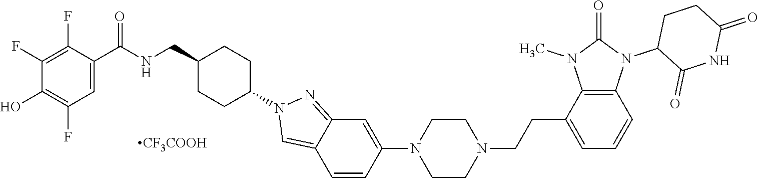 [SMILES: Cn1c(=O)n(C2CCC(=O)NC2=O)c2cccc(CCN3CCN(c4ccc5cn([C@H]6CC[C@H](CNC(=O)c7cc(F)c(O)c(F)c7F)CC6)nc5c4)CC3)c21 ; "·CF3COOH"] | N-{[(1r,4r)-4-{6-[4-(2-{1-[(3RS)- 2,6-dioxopiperidin-3-yl]-3-methyl- 2-oxo-2,3-dihydro-1H- benzimidazol-4-yl}ethyl)piperazin- 1-yl]-2H-indazol-2- yl}cyclohexyl]methyl}-2,3,5- trifluoro-4-hydroxybenzamide, trifluoroacetate salt |
| 57 | 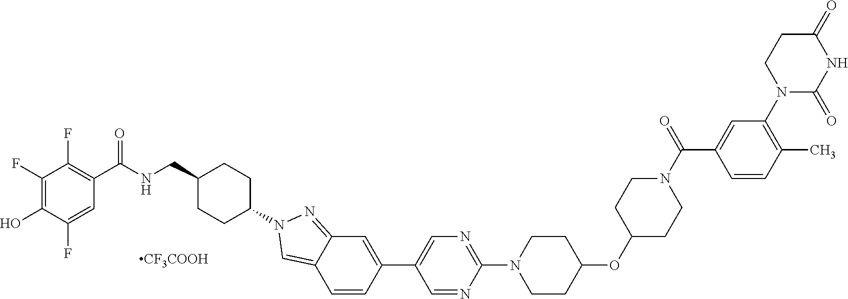 [SMILES: Cc1ccc(C(=O)N2CCC(OC3CCN(c4ncc(-c5ccc6cn([C@H]7CC[C@H](CNC(=O)c8cc(F)c(O)c(F)c8F)CC7)nc6c5)cn4)CC3)CC2)cc1N1CCC(=O)NC1=O ; "·CF3COOH"] | N-{[(1r,4r)-4-(6-{2-[4-({1-[3-(2,4- dioxo-1,3-diazinan-1-yl)-4- methylbenzoyl]piperidin-4- yl}oxy)piperidin-1-yl]pyrimidin-5- yl}-2H-indazol-2- yl)cyclohexyl]methyl}-2,3,5- trifluoro-4-hydroxybenzamide, trifluoroacetate salt |
| 58 | 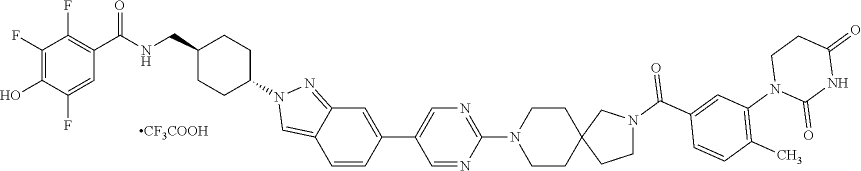 [SMILES: Cc1ccc(C(=O)N2CCC3(CCN(c4ncc(-c5ccc6cn([C@H]7CC[C@H](CNC(=O)c8cc(F)c(O)c(F)c8F)CC7)nc6c5)cn4)CC3)C2)cc1N1CCC(=O)NC1=O ; "·CF3COOH"] | N-({(1r,4r)-4-[6-(2-{2-[3-(2,4-dioxo- 1,3-diazinan-1-yl)-4- methylbenzoyl]-2,8- diazaspiro[4.5]decan-8- yl}pyrimidin-5-yl)-2H-indazol-2- yl]cyclohexyl}methyl)-2,3,5- trifluoro-4-hydroxybenzamide, trifluoroacetate salt |
| 59 | 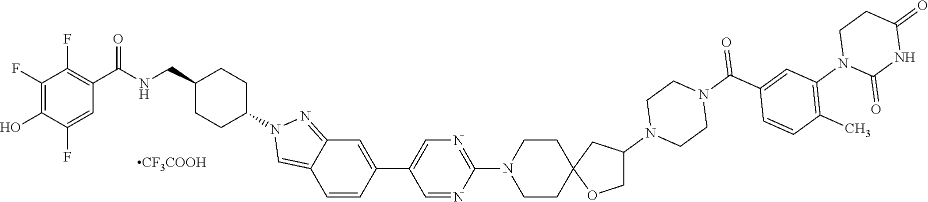 [SMILES: Cc1ccc(C(=O)N2CCN(C3COC4(CCN(c5ncc(-c6ccc7cn([C@H]8CC[C@H](CNC(=O)c9cc(F)c(O)c(F)c9F)CC8)nc7c6)cn5)CC4)C3)CC2)cc1N1CCC(=O)NC1=O ; "·CF3COOH"] | N-{[(1r,4r)-4-{6-[2-(3-{4-[3-(2,4- dioxo-1,3-diazinan-1-yl)-4- methylbenzoyl]piperazin-1-yl}-1- oxa-8-azaspiro[4.5]decan-8- yl)pyrimidin-5-yl]-2H-indazol-2- yl}cyclohexyl]methyl}-2,3,5- trifluoro-4-hydroxybenzamide, trifluoroacetate salt |
| 60 | 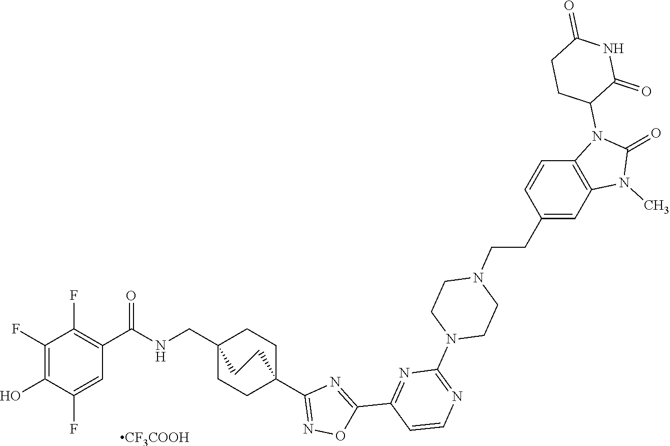 [SMILES: Cn1c(=O)n(C2CCC(=O)NC2=O)c2ccc(CCN3CCN(c4nccc(-c5nc([C@]67CC[C@](CNC(=O)c8cc(F)c(O)c(F)c8F)(CC6)CC7)no5)n4)CC3)cc21 ; "·CF3COOH"] | N-{[4-(5-{2-[4-(2-{1-[(3RS)-2,6- dioxopiperidin-3-yl]-3-methyl-2- oxo-2,3-dihydro-1H-benzimidazol- 5-yl}ethyl)piperazin-1-yl]pyrimidin- 4-yl}-1,2,4-oxadiazol-3- yl)bicyclo[2.2.2]octan-1-yl]methyl}- 2,3,5-trifluoro-4- hydroxybenzamide, trifluoroacetate salt |
| 61 | 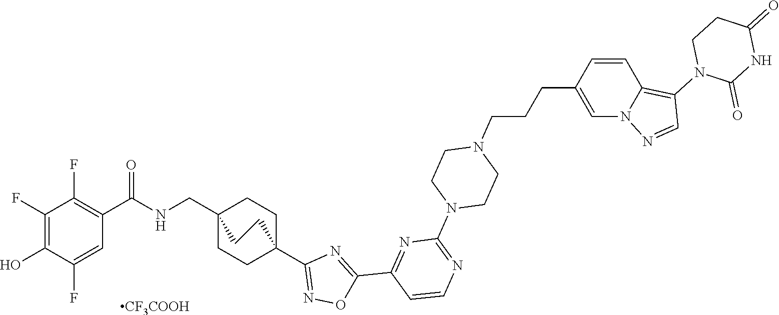 [SMILES: O=C1CCN(c2cnn3cc(CCCN4CCN(c5nccc(-c6nc([C@]78CC[C@](CNC(=O)c9cc(F)c(O)c(F)c9F)(CC7)CC8)no6)n5)CC4)ccc23)C(=O)N1 ; "·CF3COOH"] | N-[(4-{5-[2-(4-{3-[3-(2,4-dioxo-1,3- diazinan-1-yl)pyrazolo[1,5- a]pyridin-6-yl]propyl}piperazin-1- yl)pyrimidin-4-yl]-1,2,4-oxadiazol- 3-yl}bicyclo[2.2.2]octan-1- yl)methyl]-2,3,5-trifluoro-4- hydroxybenzamide, trifluoroacetate salt |
| 62 | 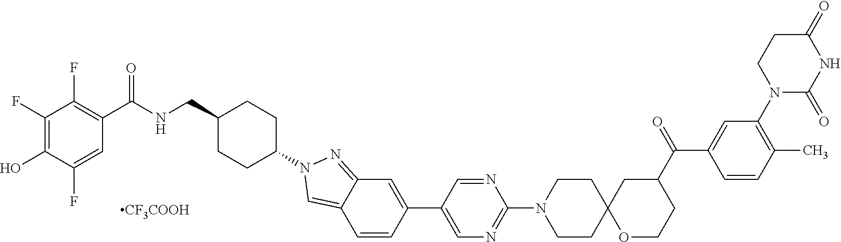 [SMILES: Cc1ccc(C(=O)C2CCOC3(CCN(c4ncc(-c5ccc6cn([C@H]7CC[C@H](CNC(=O)c8cc(F)c(O)c(F)c8F)CC7)nc6c5)cn4)CC3)C2)cc1N1CCC(=O)NC1=O ; "·CF3COOH"] | N-({(1r,4r)-4-[6-(2-{4-[3-(2,4-dioxo- 1,3-diazinan-1-yl)-4- methylbenzoyl]-1-oxa-4,9- diazaspiro[5.5]undecan-9- yl}pyrimidin-5-yl)-2H-indazol-2- yl]cyclohexyl}methyl)-2,3,5- trifluoro-4-hydroxybenzamide, trifluoroacetate salt |
| 63 | 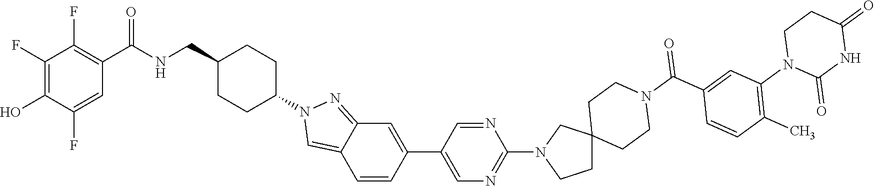 [SMILES: Cc1ccc(C(=O)N2CCC3(CC2)CCN(c2ncc(-c4ccc5cn([C@H]6CC[C@H](CNC(=O)c7cc(F)c(O)c(F)c7F)CC6)nc5c4)cn2)C3)cc1N1CCC(=O)NC1=O] | N-({(1r,4r)-4-[6-(2-{8-[3-(2,4-dioxo- 1,3-diazinan-1-yl)-4- methylbenzoyl]-2,8- diazaspiro[4.5]decan-2- yl}pyrimidin-5-yl)-2H-indazol-2- yl]cyclohexyl}methyl)-2,3,5- trifluoro-4-hydroxybenzamide |
| 64 | 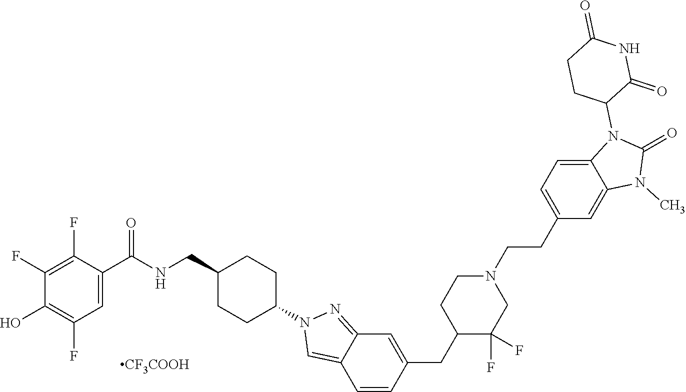 [SMILES: Cn1c(=O)n(C2CCC(=O)NC2=O)c2ccc(CCN3CCC(Cc4ccc5cn([C@H]6CC[C@H](CNC(=O)c7cc(F)c(O)c(F)c7F)CC6)nc5c4)C(F)(F)C3)cc21 ; "·CF3COOH"] | N-{[(1r,4r)-4-(6-{[1-(2-{1-[(3RS)- 2,6-dioxopiperidin-3-yl]-3-methyl- 2-oxo-2,3-dihydro-1H- benzimidazol-5-yl}ethyl)-3,3- difluoropiperidin-4-yl]methyl}-2H- indazol-2-yl)cyclohexyl]methyl}- 2,3,5-trifluoro-4- hydroxybenzamide, trifluoroacetate salt |
| 65 | 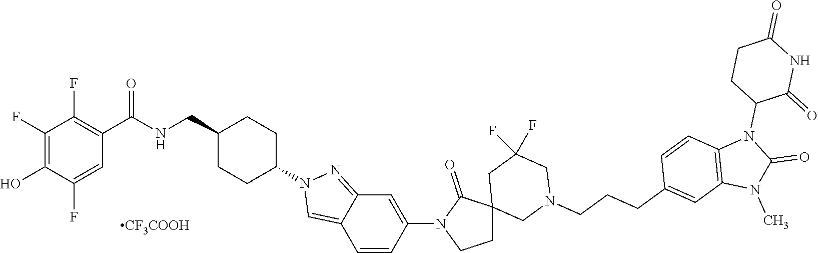 [SMILES: Cn1c(=O)n(C2CCC(=O)NC2=O)c2ccc(CCCN3CC(F)(F)CC4(CCN(c5ccc6cn([C@H]7CC[C@H](CNC(=O)c8cc(F)c(O)c(F)c8F)CC7)nc6c5)C4=O)C3)cc21 ; "·CF3COOH"] | N-{[(1r,4r)-4-{6-[7-(3-{1-[(3RS)- 2,6-dioxopiperidin-3-yl]-3-methyl- 2-oxo-2,3-dihydro-1H- benzimidazol-5-yl}propyl)-9,9- difluoro-1-oxo-2,7- diazaspiro[4.5]decan-2-yl]-2H- indazol-2-yl}cyclohexyl]methyl}- 2,3,5-trifluoro-4- hydroxybenzamide, trifluoroacetate salt |
| 66 | 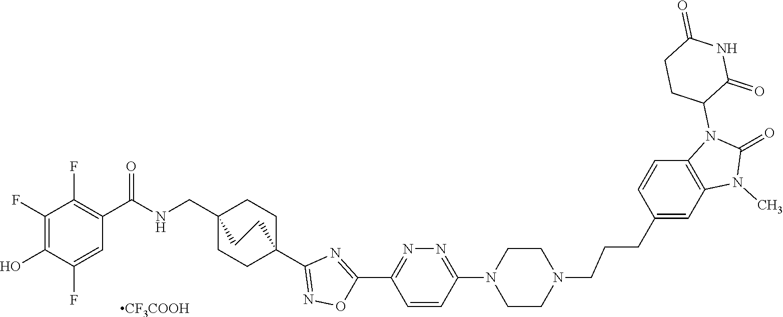 [SMILES: Cn1c(=O)n(C2CCC(=O)NC2=O)c2ccc(CCCN3CCN(c4ccc(-c5nc([C@]67CC[C@](CNC(=O)c8cc(F)c(O)c(F)c8F)(CC6)CC7)no5)nn4)CC3)cc21 ; "·CF3COOH"] | N-{[4-(5-{6-[4-(3-{1-[(3RS)-2,6- dioxopiperidin-3-yl]-3-methyl-2- oxo-2,3-dihydro-1H-benzimidazol- 5-yl}propyl)piperazin-1- yl]pyridazin-3-yl}-1,2,4-oxadiazol- 3-yl)bicyclo[2.2.2]octan-1- yl]methyl}-2,3,5-trifluoro-4- hydroxybenzamide, trifluoroacetate salt |
| 67 | 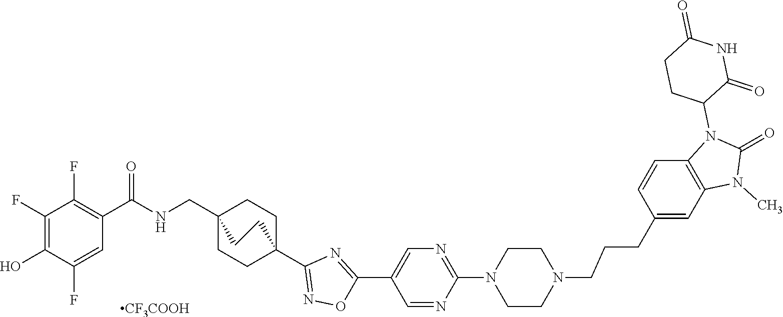 [SMILES: Cn1c(=O)n(C2CCC(=O)NC2=O)c2ccc(CCCN3CCN(c4ncc(-c5nc([C@]67CC[C@](CNC(=O)c8cc(F)c(O)c(F)c8F)(CC6)CC7)no5)cn4)CC3)cc21 ; "·CF3COOH"] | N-{[4-(5-{2-[4-(3-{1-[(3RS)-2,6- dioxopiperidin-3-yl]-3-methyl-2- oxo-2,3-dihydro-1H-benzimidazol- 5-yl}propyl)piperazin-1- yl]pyrimidin-5-yl}-1,2,4-oxadiazol- 3-yl)bicyclo[2.2.2]octan-1- yl]methyl}-2,3,5-trifluoro-4- hydroxybenzamide, trifluoroacetate salt |
| 68 | 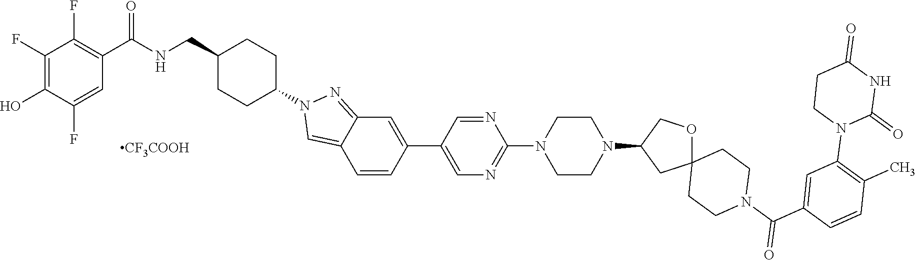 [SMILES: Cc1ccc(C(=O)N2CCC3(CC2)C[C@@H](N2CCN(c4ncc(-c5ccc6cn([C@H]7CC[C@H](CNC(=O)c8cc(F)c(O)c(F)c8F)CC7)nc6c5)cn4)CC2)CO3)cc1N1CCC(=O)NC1=O ; "·CF3COOH"] | N-{[(1r,4r)-4-{6-[2-(4-{(3R)-8-[3- (2,4-dioxo-1,3-diazinan-1-yl)-4- methylbenzoyl]-1-oxa-8- azaspiro[4.5]decan-3-yl}piperazin- 1-yl)pyrimidin-5-yl]-2H-indazol-2- yl}cyclohexyl]methyl}-2,3,5- trifluoro-4-hydroxybenzamide, trifluoroacetate salt |
| 69 | 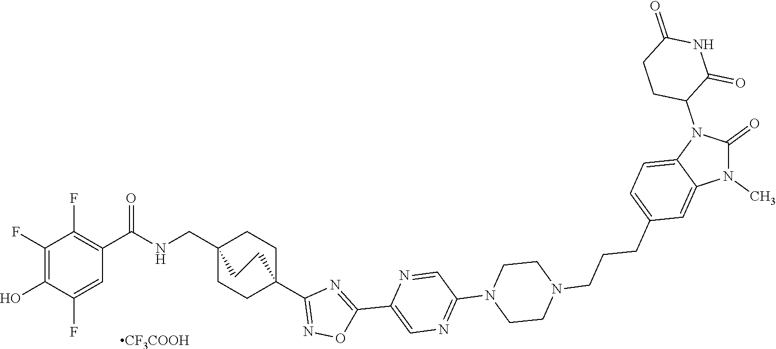 [SMILES: Cn1c(=O)n(C2CCC(=O)NC2=O)c2ccc(CCCN3CCN(c4cnc(-c5nc([C@]67CC[C@](CNC(=O)c8cc(F)c(O)c(F)c8F)(CC6)CC7)no5)cn4)CC3)cc21 ; "·CF3COOH"] | N-{[4-(5-{5-[4-(3-{1-[(3RS)-2,6- dioxopiperidin-3-yl]-3-methyl-2- oxo-2,3-dihydro-1H-benzimidazol- 5-yl}propyl)piperazin-1-yl]pyrazin- 2-yl}-1,2,4-oxadiazol-3- yl)bicyclo[2.2.2]octan-1-yl]methyl}- 2,3,5-trifluoro-4- hydroxybenzamide, trifluoroacetate salt |
| 70 | 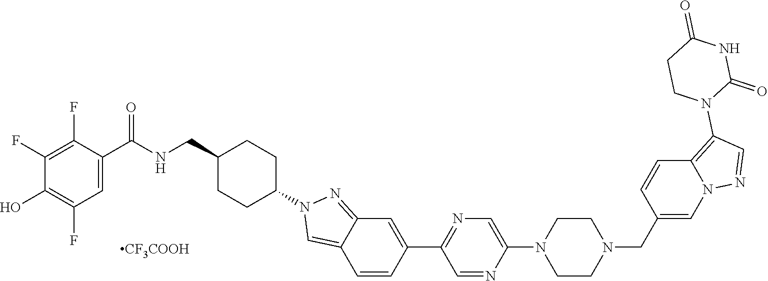 [SMILES: O=C1CCN(c2cnn3cc(CN4CCN(c5cnc(-c6ccc7cn([C@H]8CC[C@H](CNC(=O)c9cc(F)c(O)c(F)c9F)CC8)nc7c6)cn5)CC4)ccc23)C(=O)N1 ; "·CF3COOH"] | N-{[(1r,4r)-4-{6-[5-(4-{[3-(2,4- dioxo-1,3-diazinan-1- yl)pyrazolo[1,5-a]pyridin-6- yl]methyl}piperazin-1-yl)pyrazin-2- yl]-2H-indazol-2- yl}cyclohexyl]methyl}-2,3,5- trifluoro-4-hydroxybenzamide, trifluoroacetate salt |
| 71 | 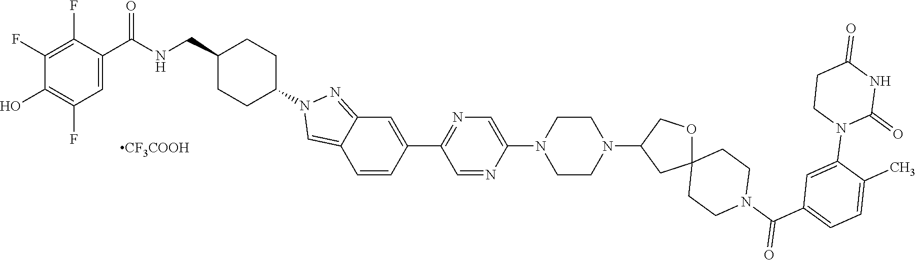 [SMILES: Cc1ccc(C(=O)N2CCC3(CC2)CC(N2CCN(c4cnc(-c5ccc6cn([C@H]7CC[C@H](CNC(=O)c8cc(F)c(O)c(F)c8F)CC7)nc6c5)cn4)CC2)CO3)cc1N1CCC(=O)NC1=O ; "·CF3COOH"] | N-{[(1r,4r)-4-{6-[5-(4-{8-[3-(2,4- dioxo-1,3-diazinan-1-yl)-4- methylbenzoyl]-1-oxa-8- azaspiro[4.5]decan-3-yl}piperazin- 1-yl)pyrazin-2-yl]-2H-indazol-2- yl}cyclohexyl]methyl}-2,3,5- trifluoro-4-hydroxybenzamide, trifluoroacetate salt |
| 72 | 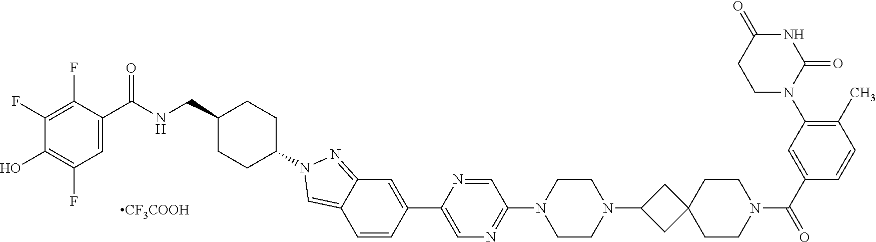 [SMILES: Cc1ccc(C(=O)N2CCC3(CC2)CC(N2CCN(c4cnc(-c5ccc6cn([C@H]7CC[C@H](CNC(=O)c8cc(F)c(O)c(F)c8F)CC7)nc6c5)cn4)CC2)C3)cc1N1CCC(=O)NC1=O ; "·CF3COOH"] | N-{[(1r,4r)-4-{6-[5-(4-{7-[3-(2,4- dioxo-1,3-diazinan-1-yl)-4- methylbenzoyl]-7- azaspiro[3.5]nonan-2-yl}piperazin- 1-yl)pyrazin-2-yl]-2H-indazol-2- yl}cyclohexyl]methyl}-2,3,5- trifluoro-4-hydroxybenzamide, trifluoroacetate salt |
| 73 | 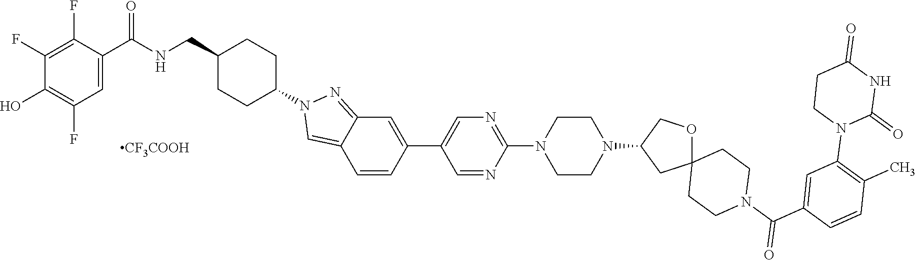 [SMILES: Cc1ccc(C(=O)N2CCC3(CC2)C[C@H](N2CCN(c4ncc(-c5ccc6cn([C@H]7CC[C@H](CNC(=O)c8cc(F)c(O)c(F)c8F)CC7)nc6c5)cn4)CC2)CO3)cc1N1CCC(=O)NC1=O ; "·CF3COOH"] | N-{[(1r,4r)-4-{6-[2-(4-{(3S)-8-[3- (2,4-dioxo-1,3-diazinan-1-yl)-4- methylbenzoyl]-1-oxa-8- azaspiro[4.5]decan-3-yl}piperazin- 1-yl)pyrimidin-5-yl]-2H-indazol-2- yl}cyclohexyl]methyl}-2,3,5- trifluoro-4-hydroxybenzamide, trifluoroacetate salt |
| 74 | 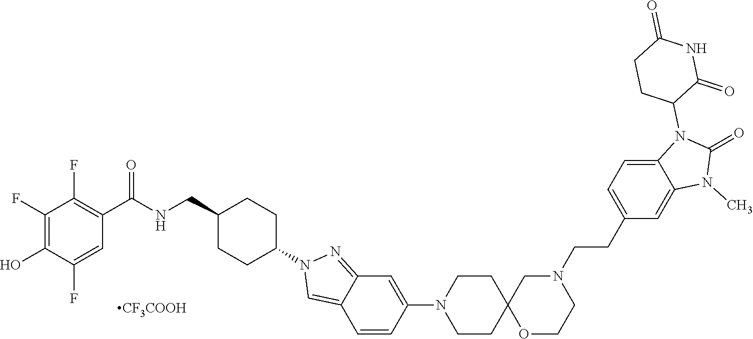 [SMILES: Cn1c(=O)n(C2CCC(=O)NC2=O)c2ccc(CCN3CCOC4(CCN(c5ccc6cn([C@H]7CC[C@H](CNC(=O)c8cc(F)c(O)c(F)c8F)CC7)nc6c5)CC4)C3)cc21 ; "·CF3COOH"] | N-{[(1r,4r)-4-{6-[4-(2-{1-[(3RS)- 2,6-dioxopiperidin-3-yl]-3-methyl- 2-oxo-2,3-dihydro-1H- benzimidazol-5-yl}ethyl)-1-oxa- 4,9-diazaspiro[5.5]undecan-9-yl]- 2H-indazol-2- yl}cyclohexyl]methyl}-2,3,5- trifluoro-4-hydroxybenzamide, trifluoroacetate salt |
| 75 | 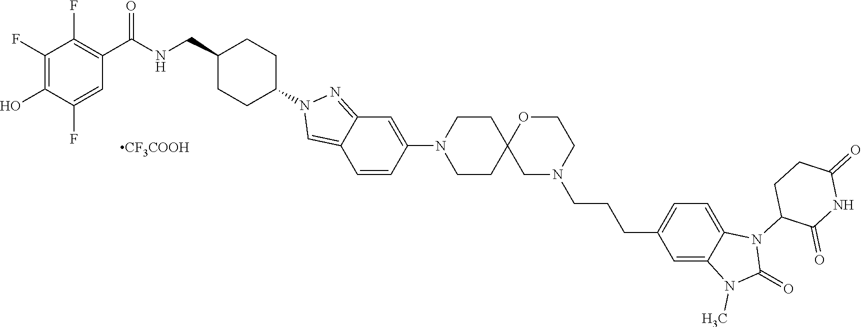 [SMILES: Cn1c(=O)n(C2CCC(=O)NC2=O)c2ccc(CCCN3CCOC4(CCN(c5ccc6cn([C@H]7CC[C@H](CNC(=O)c8cc(F)c(O)c(F)c8F)CC7)nc6c5)CC4)C3)cc21 ; "·CF3COOH"] | N-{[(1r,4r)-4-{6-[4-(3-{1-[(3RS)- 2,6-dioxopiperidin-3-yl]-3-methyl- 2-oxo-2,3-dihydro-1H- benzimidazol-5-yl}propyl)-1-oxa- 4,9-diazaspiro[5.5]undecan-9-yl]- 2H-indazol-2- yl}cyclohexyl]methyl}-2,3,5- trifluoro-4-hydroxybenzamide, trifluoroacetate salt |
| 76 | 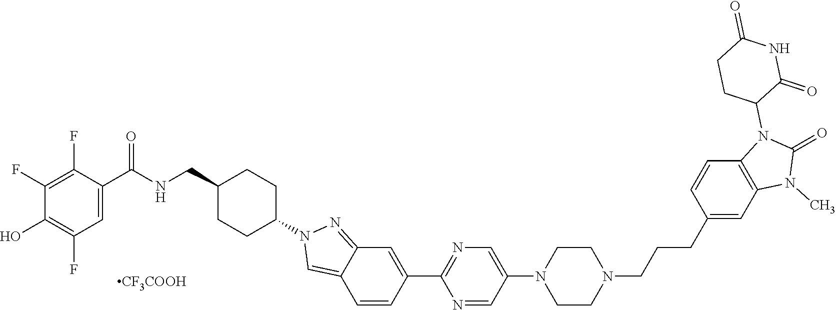 [SMILES: Cn1c(=O)n(C2CCC(=O)NC2=O)c2ccc(CCCN3CCN(c4cnc(-c5ccc6cn([C@H]7CC[C@H](CNC(=O)c8cc(F)c(O)c(F)c8F)CC7)nc6c5)nc4)CC3)cc21 ; "·CF3COOH"] | N-{[(1r,4r)-4-(6-{5-[4-(3-{1-[(3RS)- 2,6-dioxopiperidin-3-yl]-3-methyl- 2-oxo-2,3-dihydro-1H- benzimidazol-5- yl}propyl)piperazin-1-yl]pyrimidin- 2-yl}-2H-indazol-2- yl)cyclohexyl]methyl}-2,3,5- trifluoro-4-hydroxybenzamide, trifluoroacetate salt |
| 77 | 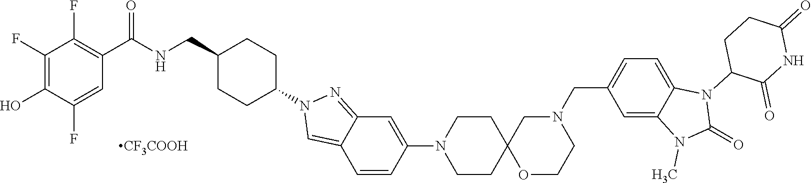 [SMILES: Cn1c(=O)n(C2CCC(=O)NC2=O)c2ccc(CN3CCOC4(CCN(c5ccc6cn([C@H]7CC[C@H](CNC(=O)c8cc(F)c(O)c(F)c8F)CC7)nc6c5)CC4)C3)cc21 ; "·CF3COOH"] | N-{[(1r,4r)-4-{6-[4-({1-[(3RS)-2,6- dioxopiperidin-3-yl]-3-methyl-2- oxo-2,3-dihydro-1H-benzimidazol- 5-yl}methyl)-1-oxa-4,9- diazaspiro[5.5]undecan-9-yl]-2H- indazol-2-yl}cyclohexyl]methyl}- 2,3,5-trifluoro-4- hydroxybenzamide, trifluoroacetate salt |
| 78 | 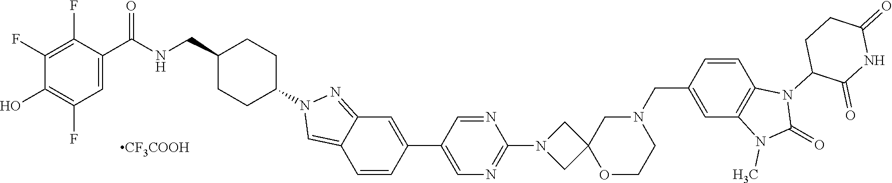 [SMILES: Cn1c(=O)n(C2CCC(=O)NC2=O)c2ccc(CN3CCOC4(C3)CN(c3ncc(-c5ccc6cn([C@H]7CC[C@H](CNC(=O)c8cc(F)c(O)c(F)c8F)CC7)nc6c5)cn3)C4)cc21 ; "·CF3COOH"] | N-{[(1r,4r)-4-(6-{2-[8-({1-[(3RS)- 2,6-dioxopiperidin-3-yl]-3-methyl- 2-oxo-2,3-dihydro-1H- benzimidazol-5-yl}methyl)-5-oxa- 2,8-diazaspiro[3.5]nonan-2- yl]pyrimidin-5-yl}-2H-indazol-2- yl)cyclohexyl]methyl}-2,3,5- trifluoro-4-hydroxybenzamide, trifluoroacetate salt |
| 79 | 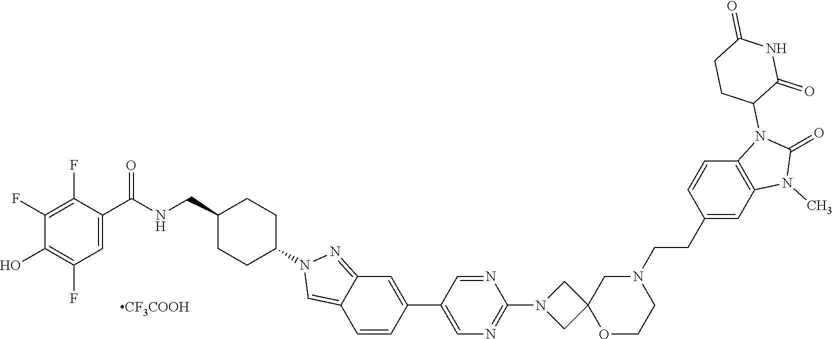 [SMILES: Cn1c(=O)n(C2CCC(=O)NC2=O)c2ccc(CCN3CCOC4(C3)CN(c3ncc(-c5ccc6cn([C@H]7CC[C@H](CNC(=O)c8cc(F)c(O)c(F)c8F)CC7)nc6c5)cn3)C4)cc21 ; "·CF3COOH"] | N-{[(1r,4r)-4-(6-{2-[8-(2-{1-[(3RS)- 2,6-dioxopiperidin-3-yl]-3-methyl- 2-oxo-2,3-dihydro-1H- benzimidazol-5-yl}ethyl)-5-oxa- 2,8-diazaspiro[3.5]nonan-2- yl]pyrimidin-5-yl}-2H-indazol-2- yl)cyclohexyl]methyl}-2,3,5- trifluoro-4-hydroxybenzamide, trifluoroacetate salt |
| 80 | 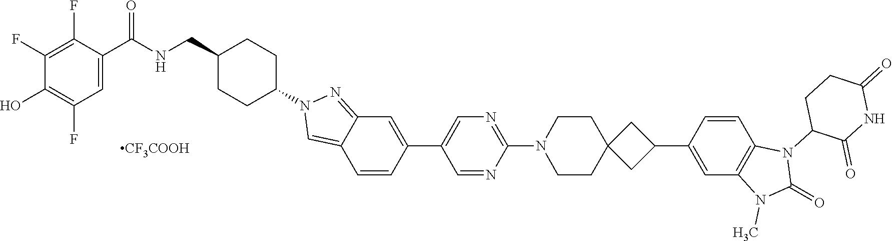 [SMILES: Cn1c(=O)n(C2CCC(=O)NC2=O)c2ccc(C3CC4(CCN(c5ncc(-c6ccc7cn([C@H]8CC[C@H](CNC(=O)c9cc(F)c(O)c(F)c9F)CC8)nc7c6)cn5)CC4)C3)cc21 ; "·CF3COOH"] | N-{[(1r,4r)-4-{6-[2-(2-{1-[(3RS)- 2,6-dioxopiperidin-3-yl]-3-methyl- 2-oxo-2,3-dihydro-1H- benzimidazol-5-yl}-7- azaspiro[3.5]nonan-7-yl)pyrimidin- 5-yl]-2H-indazol-2- yl}cyclohexyl]methyl}-2,3,5- trifluoro-4-hydroxybenzamide, trifluoroacetate salt |
| 81 | 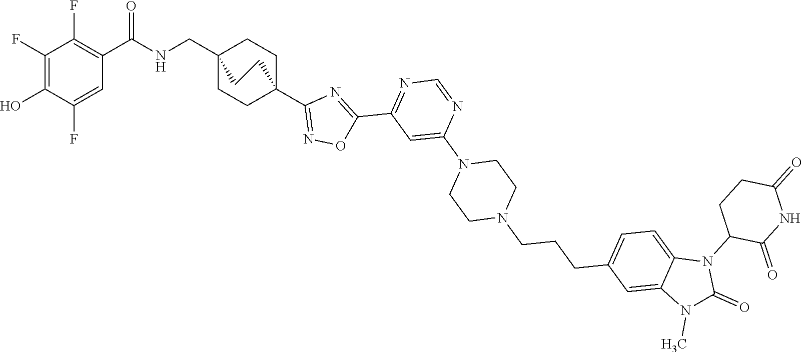 [SMILES: Cn1c(=O)n(C2CCC(=O)NC2=O)c2ccc(CCCN3CCN(c4cc(-c5nc([C@]67CC[C@](CNC(=O)c8cc(F)c(O)c(F)c8F)(CC6)CC7)no5)ncn4)CC3)cc21] | N-{[4-(5-{3-[4-(3-{1-[(3RS)-2,6- dioxopiperidin-3-yl]-3-methyl-2- oxo-2,3-dihydro-1H-benzimidazol- 5-yl}propyl)piperazin-1-yl]phenyl}- 1,2,4-oxadiazol-3- yl)bicyclo[2.2.2]octan-1-yl]methyl}- 2,3,5-trifluoro-4- hydroxybenzamide |
| 82 | 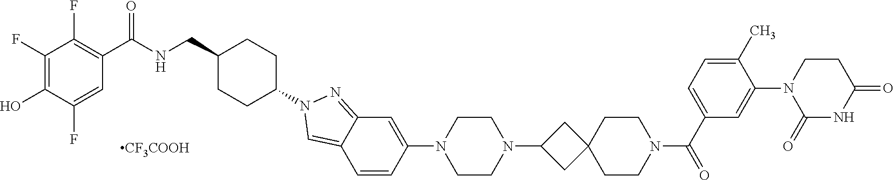 [SMILES: Cc1ccc(C(=O)N2CCC3(CC2)CC(N2CCN(c4ccc5cn([C@H]6CC[C@H](CNC(=O)c7cc(F)c(O)c(F)c7F)CC6)nc5c4)CC2)C3)cc1N1CCC(=O)NC1=O ; "·CF3COOH"] | N-({(1r,4r)-4-[6-(4-{7-[3-(2,4-dioxo- 1,3-diazinan-1-yl)-4- methylbenzoyl]-7- azaspiro[3.5]nonan-2-yl}piperazin- 1-yl)-2H-indazol-2- yl]cyclohexyl}methyl)-2,3,5- trifluoro-4-hydroxybenzamide, trifluoroacetate salt |
| 83 | 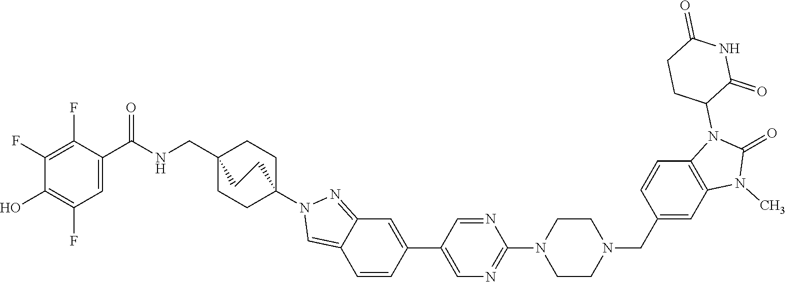 [SMILES: Cn1c(=O)n(C2CCC(=O)NC2=O)c2ccc(CN3CCN(c4ncc(-c5ccc6cn([C@]78CC[C@](CNC(=O)c9cc(F)c(O)c(F)c9F)(CC7)CC8)nc6c5)cn4)CC3)cc21] | N-{[4-(6-{2-[4-({1-[(3RS)-2,6- dioxopiperidin-3-yl]-3-methyl-2- oxo-2,3-dihydro-1H-benzimidazol- 5-yl}methyl)piperazin-1- yl]pyrimidin-5-yl}-2H-indazol-2- yl)bicyclo[2.2.2]octan-1-yl]methyl}- 2,3,5-trifluoro-4- hydroxybenzamide |
| 84 | 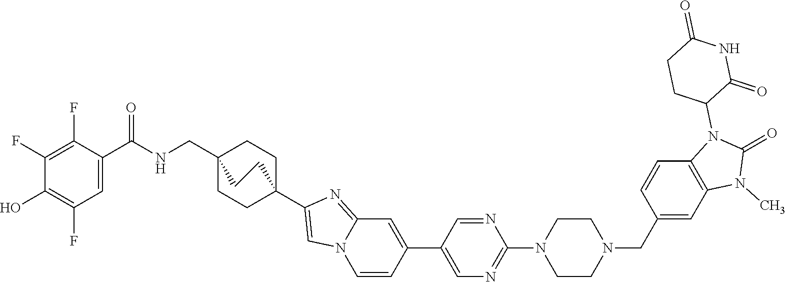 [SMILES: Cn1c(=O)n(C2CCC(=O)NC2=O)c2ccc(CN3CCN(c4ncc(-c5ccn6cc([C@]78CC[C@](CNC(=O)c9cc(F)c(O)c(F)c9F)(CC7)CC8)nc6c5)cn4)CC3)cc21] | N-{[4-(7-{2-[4-({1-[(3RS)-2,6- dioxopiperidin-3-yl]-3-methyl-2- oxo-2,3-dihydro-1H-benzimidazol- 5-yl}methyl)piperazin-1- yl]pyrimidin-5-yl}imidazo[1,2- a]pyridin-2-yl)bicyclo[2.2.2]octan- 1-yl]methyl}-2,3,5-trifluoro-4- hydroxybenzamide |
| 85 | 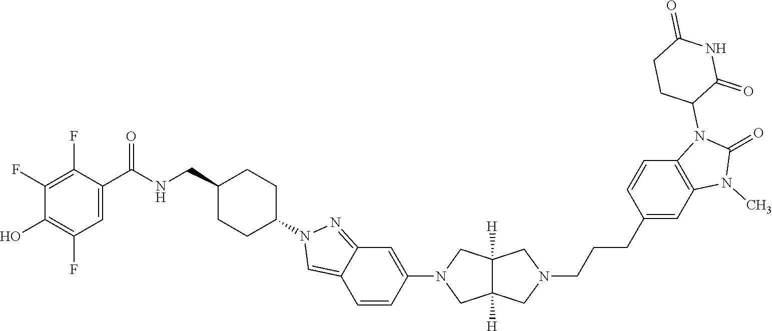 [SMILES: Cn1c(=O)n(C2CCC(=O)NC2=O)c2ccc(CCCN3C[C@@H]4CN(c5ccc6cn([C@H]7CC[C@H](CNC(=O)c8cc(F)c(O)c(F)c8F)CC7)nc6c5)C[C@@H]4C3)cc21] | N-{[(1r,4r)-4-{6-[(3aR,6aS)-5-(3- {1-[(3RS)-2,6-dioxopiperidin-3-yl]- 3-methyl-2-oxo-2,3-dihydro-1H- benzimidazol-5- yl}propyl)hexahydropyrrolo[3,4- c]pyrrol-2(1H)-yl]-2H-indazol-2- yl}cyclohexyl]methyl}-2,3,5- trifluoro-4-hydroxybenzamide |
| 86 | 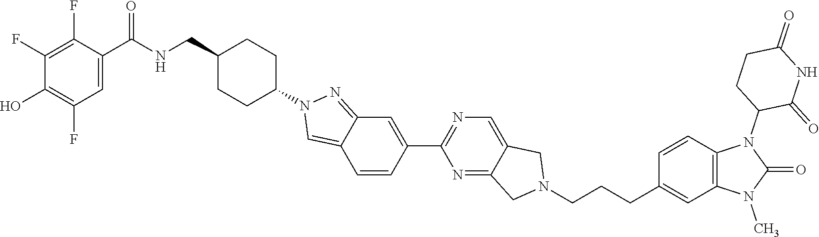 [SMILES: Cn1c(=O)n(C2CCC(=O)NC2=O)c2ccc(CCCN3Cc4cnc(-c5ccc6cn([C@H]7CC[C@H](CNC(=O)c8cc(F)c(O)c(F)c8F)CC7)nc6c5)nc4C3)cc21] | N-{[(1r,4r)-4-{6-[6-(3-{1-[(3RS)- 2,6-dioxopiperidin-3-yl]-3-methyl- 2-oxo-2,3-dihydro-1H- benzimidazol-5-yl}propyl)-6,7- dihydro-5H-pyrrolo[3,4- d]pyrimidin-2-yl]-2H-indazol-2- yl}cyclohexyl]methyl}-2,3,5- trifluoro-4-hydroxybenzamide |
| 87 | 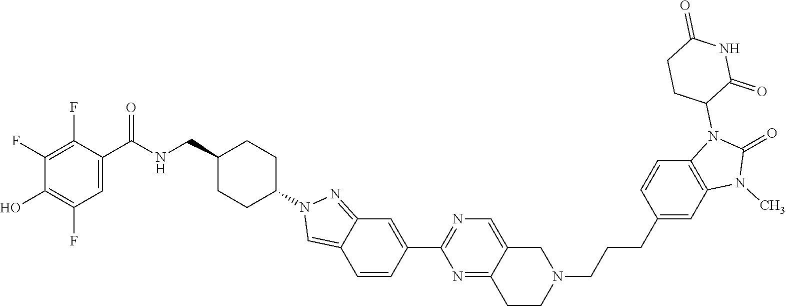 [SMILES: Cn1c(=O)n(C2CCC(=O)NC2=O)c2ccc(CCCN3CCc4nc(-c5ccc6cn([C@H]7CC[C@H](CNC(=O)c8cc(F)c(O)c(F)c8F)CC7)nc6c5)ncc4C3)cc21] | N-{[(1r,4r)-4-{6-[6-(3-{1-[(3RS)- 2,6-dioxopiperidin-3-yl]-3-methyl- 2-oxo-2,3-dihydro-1H- benzimidazol-5-yl}propyl)-5,6,7,8- tetrahydropyrido[4,3-d]pyrimidin- 2-yl]-2H-indazol-2- yl}cyclohexyl]methyl}-2,3,5- trifluoro-4-hydroxybenzamide |
| 88 | 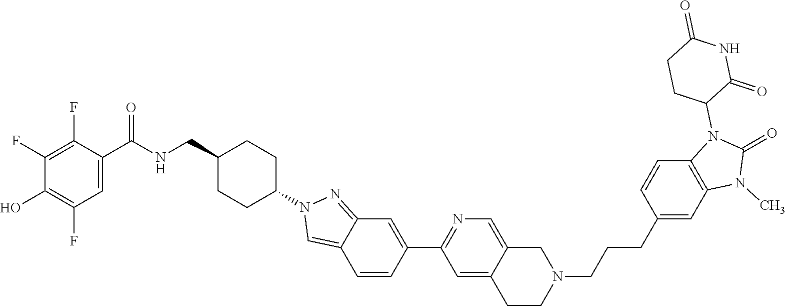 [SMILES: Cn1c(=O)n(C2CCC(=O)NC2=O)c2ccc(CCCN3CCc4cc(-c5ccc6cn([C@H]7CC[C@H](CNC(=O)c8cc(F)c(O)c(F)c8F)CC7)nc6c5)ncc4C3)cc21] | N-{[(1r,4r)-4-{6-[7-(3-{1-[(3RS)- 2,6-dioxopiperidin-3-yl]-3-methyl- 2-oxo-2,3-dihydro-1H- benzimidazol-5-yl}propyl)-5,6,7,8- tetrahydro-2,7-naphthyridin-3-yl]- 2H-indazol-2- yl}cyclohexyl]methyl}-2,3,5- trifluoro-4-hydroxybenzamide |
| 89 | 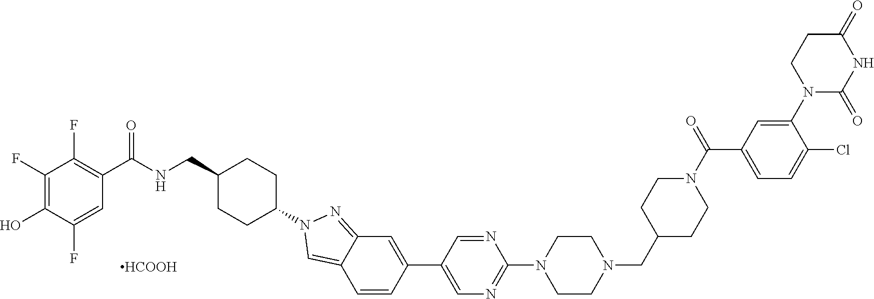 [SMILES: O=C1CCN(c2cc(C(=O)N3CCC(CN4CCN(c5ncc(-c6ccc7cn([C@H]8CC[C@H](CNC(=O)c9cc(F)c(O)c(F)c9F)CC8)nc7c6)cn5)CC4)CC3)ccc2Cl)C(=O)N1 ; "·HCOOH"] | N-{[(1r,4r)-4-(6-{2-[4-({1-[4-chloro- 3-(2,4-dioxo-1,3-diazinan-1- yl)benzoyl]piperidin-4- yl}methyl)piperazin-1-yl]pyrimidin- 5-yl}-2H-indazol-2- yl)cyclohexyl]methyl}-2,3,5- trifluoro-4-hydroxybenzamide, formate salt |
| 90 | 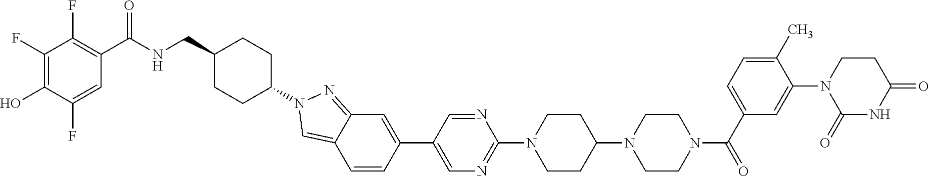 [SMILES: Cc1ccc(C(=O)N2CCN(C3CCN(c4ncc(-c5ccc6cn([C@H]7CC[C@H](CNC(=O)c8cc(F)c(O)c(F)c8F)CC7)nc6c5)cn4)CC3)CC2)cc1N1CCC(=O)NC1=O] | N-{[(1r,4r)-4-{6-[2-(4-{4-[3-(2,4- dioxo-1,3-diazinan-1-yl)-4- methylbenzoyl]piperazin-1- yl}piperidin-1-yl)pyrimidin-5-yl]-2H- indazol-2-yl}cyclohexyl]methyl}- 2,3,5-trifluoro-4- hydroxybenzamide |
| 91 | 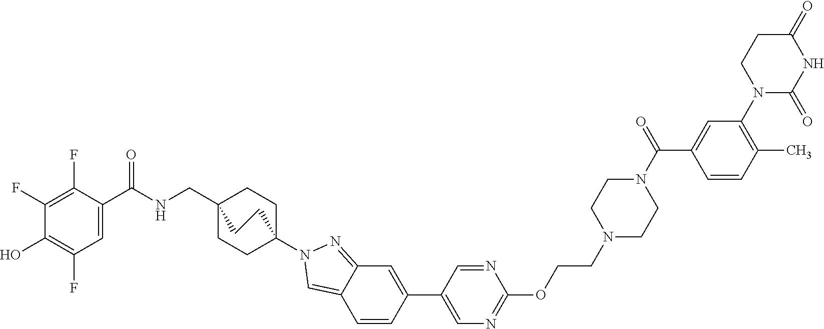 [SMILES: Cc1ccc(C(=O)N2CCN(CCOc3ncc(-c4ccc5cn([C@]67CC[C@](CNC(=O)c8cc(F)c(O)c(F)c8F)(CC6)CC7)nc5c4)cn3)CC2)cc1N1CCC(=O)NC1=O] | N-[(4-{6-[2-(2-{4-[3-(2,4-dioxo-1,3- diazinan-1-yl)-4- methylbenzoyl]piperazin-1- yl}ethoxy)pyrimidin-5-yl]-2H- indazol-2-yl}bicyclo[2.2.2]octan-1- yl)methyl]-2,3,5-trifluoro-4- hydroxybenzamide |
| 92 | 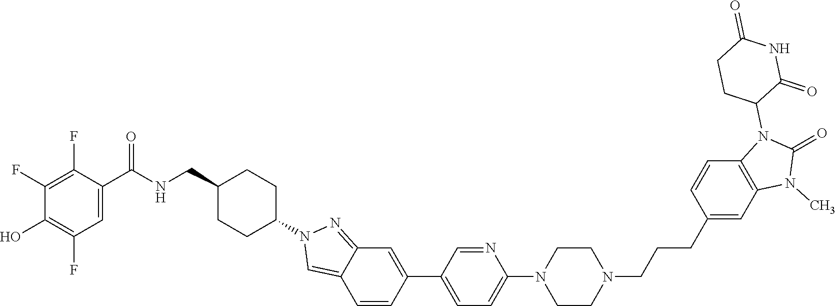 [SMILES: Cn1c(=O)n(C2CCC(=O)NC2=O)c2ccc(CCCN3CCN(c4ccc(-c5ccc6cn([C@H]7CC[C@H](CNC(=O)c8cc(F)c(O)c(F)c8F)CC7)nc6c5)cn4)CC3)cc21] | N-{[(1r,4r)-4-(6-{6-[4-(3-{1-[(3RS)- 2,6-dioxopiperidin-3-yl]-3-methyl- 2-oxo-2,3-dihydro-1H- benzimidazol-5- yl}propyl)piperazin-1-yl]pyridin-3- yl}-2H-indazol-2- yl)cyclohexyl]methyl}-2,3,5- trifluoro-4-hydroxybenzamide |
| 93 | 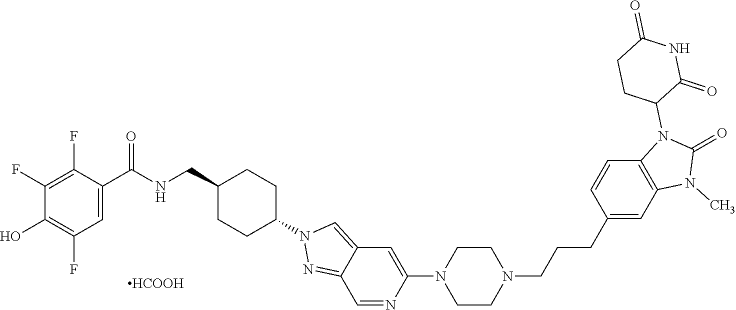 [SMILES: Cn1c(=O)n(C2CCC(=O)NC2=O)c2ccc(CCCN3CCN(c4cc5cn([C@H]6CC[C@H](CNC(=O)c7cc(F)c(O)c(F)c7F)CC6)nc5cn4)CC3)cc21 ; "·HCOOH"] | N-{[(1r,4r)-4-{5-[4-(3-{1-[(3RS)- 2,6-dioxopiperidin-3-yl]-3-methyl- 2-oxo-2,3-dihydro-1H- benzimidazol-5- yl}propyl)piperazin-1-yl]-2H- pyrazolo[3,4-c]pyridin-2- yl}cyclohexyl]methyl}-2,3,5- trifluoro-4-hydroxybenzamide, formate salt |
| 94 | 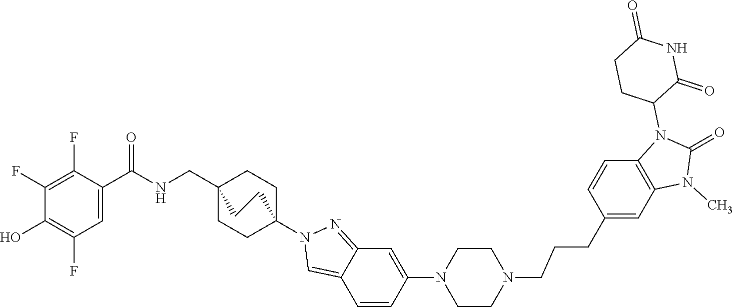 [SMILES: Cn1c(=O)n(C2CCC(=O)NC2=O)c2ccc(CCCN3CCN(c4ccc5cn([C@]67CC[C@](CNC(=O)c8cc(F)c(O)c(F)c8F)(CC6)CC7)nc5c4)CC3)cc21] | N-[(4-{6-[4-(3-{1-[(3RS)-2,6- dioxopiperidin-3-yl]-3-methyl-2- oxo-2,3-dihydro-1H-benzimidazol- 5-yl}propyl)piperazin-1-yl]-2H- indazol-2-yl}bicyclo[2.2.2]octan-1- yl)methyl]-2,3,5-trifluoro-4- hydroxybenzamide |
| 95 | 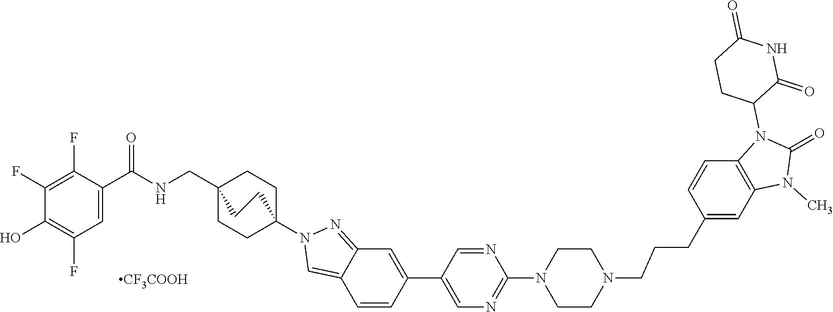 [SMILES: Cn1c(=O)n(C2CCC(=O)NC2=O)c2ccc(CCCN3CCN(c4ncc(-c5ccc6cn([C@]78CC[C@](CNC(=O)c9cc(F)c(O)c(F)c9F)(CC7)CC8)nc6c5)cn4)CC3)cc21 ; "·CF3COOH"] | N-{[4-(6-{2-[4-(3-{1-[(3RS)-2,6- dioxopiperidin-3-yl]-3-methyl-2- oxo-2,3-dihydro-1H-benzimidazol- 5-yl}propyl)piperazin-1- yl]pyrimidin-5-yl}-2H-indazol-2- yl)bicyclo[2.2.2]octan-1-yl]methyl}- 2,3,5-trifluoro-4- hydroxybenzamide, trifluoroacetate salt |
| 96 | 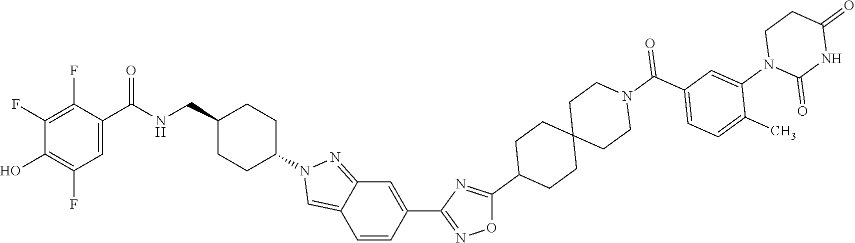 [SMILES: Cc1ccc(C(=O)N2CCC3(CCC(c4nc(-c5ccc6cn([C@H]7CC[C@H](CNC(=O)c8cc(F)c(O)c(F)c8F)CC7)nc6c5)no4)CC3)CC2)cc1N1CCC(=O)NC1=O] | N-({(1r,4r)-4-[6-(5-{3-[3-(2,4-dioxo- 1,3-diazinan-1-yl)-4- methylbenzoyl]-3- azaspiro[5.5]undecan-9-yl}-1,2,4- oxadiazol-3-yl)-2H-indazol-2- yl]cyclohexyl}methyl)-2,3,5- trifluoro-4-hydroxybenzamide |
| 97 | 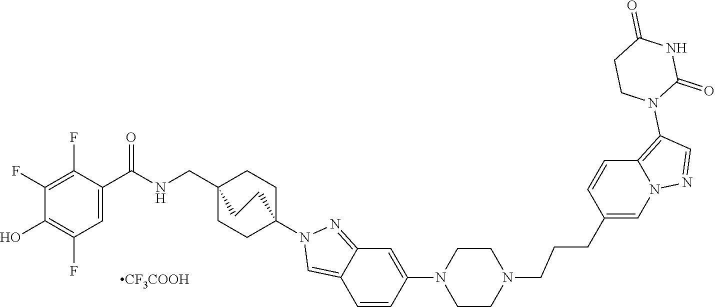 [SMILES: O=C1CCN(c2cnn3cc(CCCN4CCN(c5ccc6cn([C@]78CC[C@](CNC(=O)c9cc(F)c(O)c(F)c9F)(CC7)CC8)nc6c5)CC4)ccc23)C(=O)N1 ; "·CF3COOH"] | N-({4-[6-(4-{3-[3-(2,4-dioxo-1,3- diazinan-1-yl)pyrazolo[1,5- a]pyridin-6-yl]propyl}piperazin-1- yl)-2H-indazol-2- yl]bicyclo[2.2.2]octan-1-yl}methyl)- 2,3,5-trifluoro-4- hydroxybenzamide, trifluoroacetate salt |
| 98 | 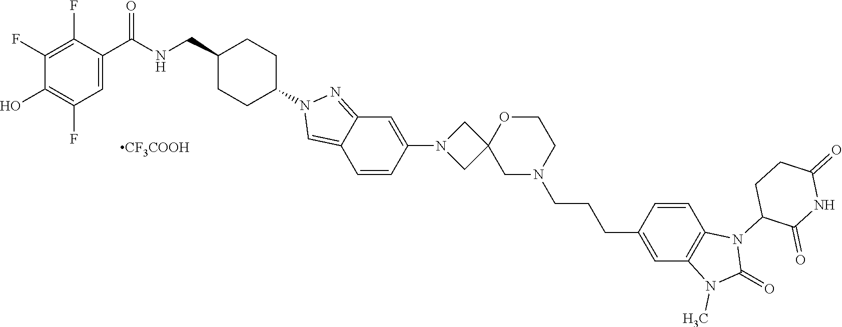 [SMILES: Cn1c(=O)n(C2CCC(=O)NC2=O)c2ccc(CCCN3CCOC4(C3)CN(c3ccc5cn([C@H]6CC[C@H](CNC(=O)c7cc(F)c(O)c(F)c7F)CC6)nc5c3)C4)cc21 ; "·CF3COOH"] | N-{[(1r,4r)-4-{6-[8-(3-{1-[(3RS)- 2,6-dioxopiperidin-3-yl]-3-methyl- 2-oxo-2,3-dihydro-1H- benzimidazol-5-yl}propyl)-5-oxa- 2,8-diazaspiro[3.5]nonan-2-yl]-2H- indazol-2-yl}cyclohexyl]methyl}- 2,3,5-trifluoro-4- hydroxybenzamide, trifluoroacetate salt |
| 99 | 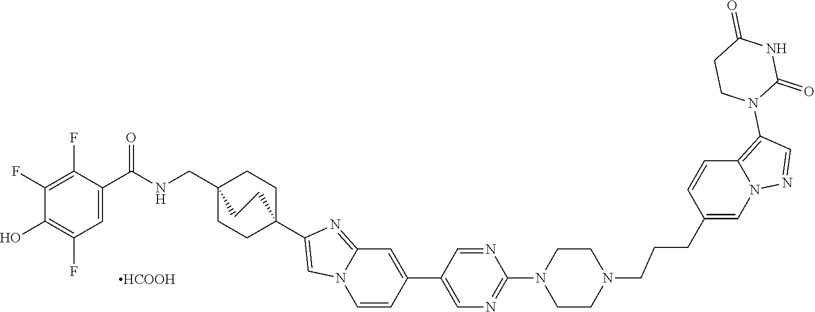 [SMILES: O=C1CCN(c2cnn3cc(CCCN4CCN(c5ncc(-c6ccn7cc([C@]89CC[C@](CNC(=O)c%10cc(F)c(O)c(F)c%10F)(CC8)CC9)nc7c6)cn5)CC4)ccc23)C(=O)N1 ; "·HCOOH"] | N-[(4-{7-[2-(4-{3-[3-(2,4-dioxo-1,3- diazinan-1-yl)pyrazolo[1,5- a]pyridin-6-yl]propyl}piperazin-1- yl)pyrimidin-5-yl]imidazo[1,2- a]pyridin-2-yl}bicyclo[2.2.2]octan- 1-yl)methyl]-2,3,5-trifluoro-4- hydroxybenzamide, formate salt |
| 100 | 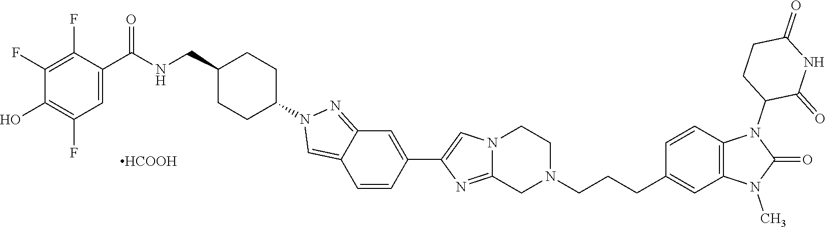 [SMILES: Cn1c(=O)n(C2CCC(=O)NC2=O)c2ccc(CCCN3CCn4cc(-c5ccc6cn([C@H]7CC[C@H](CNC(=O)c8cc(F)c(O)c(F)c8F)CC7)nc6c5)nc4C3)cc21 ; "·HCOOH"] | N-{[(1r,4r)-4-{6-[7-(3-{1-[(3RS)- 2,6-dioxopiperidin-3-yl]-3-methyl- 2-oxo-2,3-dihydro-1H- benzimidazol-5-yl}propyl)-5,6,7,8- tetrahydroimidazo[1,2-a]pyrazin-2- yl]-2H-indazol-2- yl}cyclohexyl]methyl}-2,3,5- trifluoro-4-hydroxybenzamide, formate salt |
| 101 | 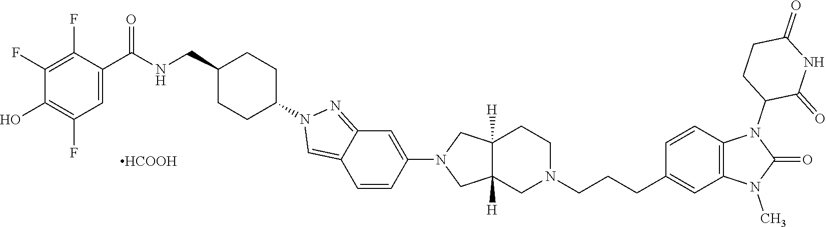 [SMILES: Cn1c(=O)n(C2CCC(=O)NC2=O)c2ccc(CCCN3CC[C@@H]4CN(c5ccc6cn([C@H]7CC[C@H](CNC(=O)c8cc(F)c(O)c(F)c8F)CC7)nc6c5)C[C@H]4C3)cc21 ; "·HCOOH"] | N-{[(1r,4r)-4-{6-[(3aR,7aS)-5-(3- {1-[(3RS)-2,6-dioxopiperidin-3-yl]- 3-methyl-2-oxo-2,3-dihydro-1H- benzimidazol-5- yl}propyl)octahydro-2H- pyrrolo[3,4-c]pyridin-2-yl]-2H- indazol-2-yl}cyclohexyl]methyl}- 2,3,5-trifluoro-4- hydroxybenzamide, formate salt |
| 102 | 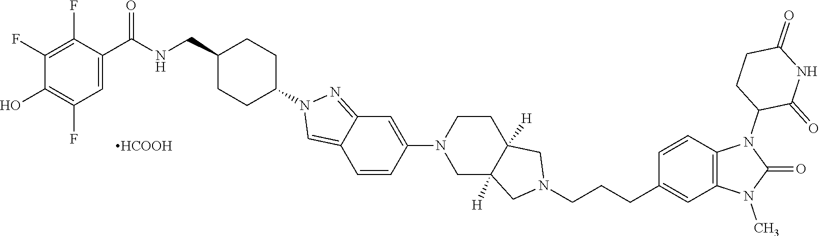 [SMILES: Cn1c(=O)n(C2CCC(=O)NC2=O)c2ccc(CCCN3C[C@@H]4CCN(c5ccc6cn([C@H]7CC[C@H](CNC(=O)c8cc(F)c(O)c(F)c8F)CC7)nc6c5)C[C@@H]4C3)cc21 ; "·HCOOH"] | N-{[(1r,4r)-4-{6-[(3aS,7aR)-2-(3- {1-[(3RS)-2,6-dioxopiperidin-3-yl]- 3-methyl-2-oxo-2,3-dihydro-1H- benzimidazol-5- yl}propyl)octahydro-5H- pyrrolo[3,4-c]pyridin-5-yl]-2H- indazol-2-yl}cyclohexyl]methyl}- 2,3,5-trifluoro-4- hydroxybenzamide, formate salt |
| 103 | 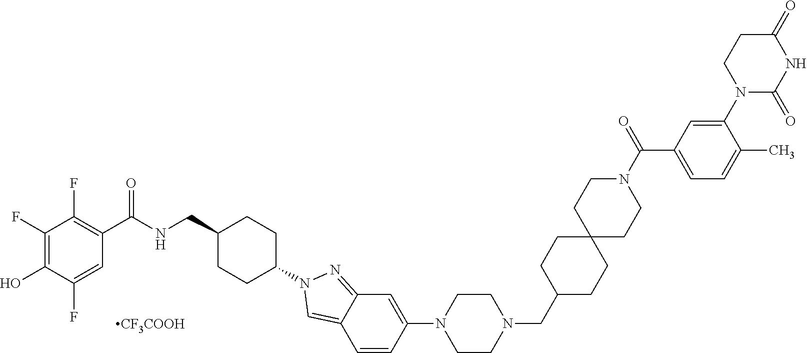 | N-{[(1r,4r)-4-{6-[4-({3-[3-(2,4- dioxo-1,3-diazinan-1-yl)-4- methylbenzoyl]-3- azaspiro[5.5]undecan-9- yl}methyl)piperazin-1-yl]-2H- indazol-2-yl}cyclohexyl]methyl}- 2,3,5-trifluoro-4- hydroxybenzamide, trifluoroacetate salt |
| 104 | 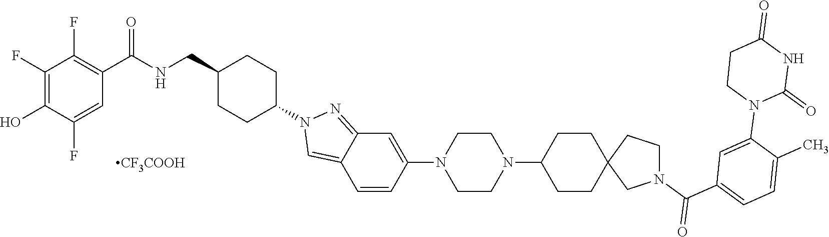 | N-({(1r,4r)-4-[6-(4-{2-[3-(2,4-dioxo- 1,3-diazinan-1-yl)-4- methylbenzoyl]-2- azaspiro[4.5]decan-8-yl}piperazin- 1-yl)-2H-indazol-2- yl]cyclohexyl}methyl)-2,3,5- trifluoro-4-hydroxybenzamide, trifluoroacetate salt |
| 105 | 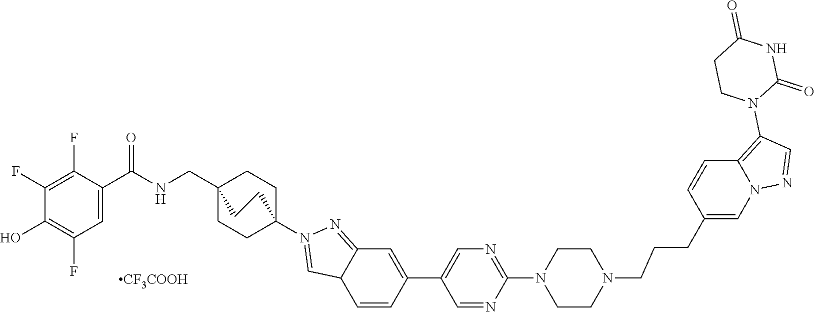 | N-[(4-{6-[2-(4-{3-[3-(2,4-dioxo-1,3- diazinan-1-yl)pyrazolo[1,5- a]pyridin-6-yl]propyl}piperazin-1- yl)pyrimidin-5-yl]-2H-indazol-2- yl}bicyclo[2.2.2]octan-1-yl)methyl]- 2,3,5-trifluoro-4- hydroxybenzamide, trifluoroacetate salt |
| 106 | 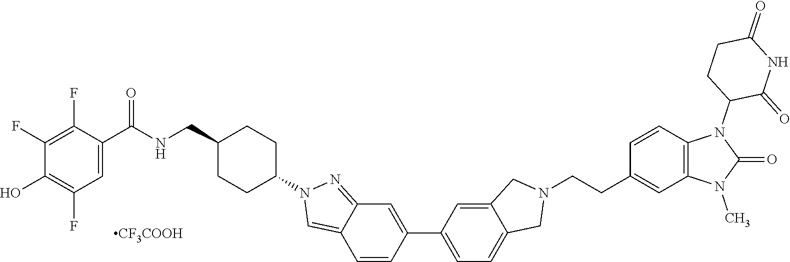 | N-{[(1r,4r)-4-{6-[2-(2-{1-[(3RS)- 2,6-dioxopiperidin-3-yl]-3-methyl- 2-oxo-2,3-dihydro-1H- benzimidazol-5-yl}ethyl)-2,3- dihydro-1H-isoindol-5-yl]-2H- indazol-2-yl}cyclohexyl]methyl}- 2,3,5-trifluoro-4- hydroxybenzamide, trifluoroacetate salt |
| 107 | 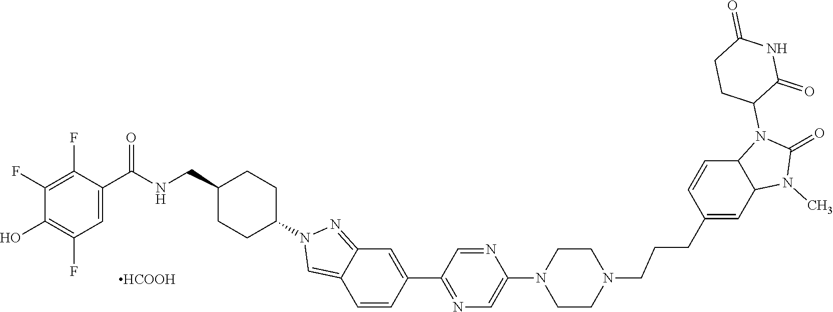 [SMILES: CN1C(=O)N(C2CCC(=O)NC2=O)C2C=CC(CCCN3CCN(c4cnc(-c5ccc6cn([C@H]7CC[C@H](CNC(=O)c8cc(F)c(O)c(F)c8F)CC7)nc6c5)cn4)CC3)=CC21 ; "·HCOOH"] | N-{[(1r,4r)-4-(6-{5-[4-(3-{1-[(3RS)- 2,6-dioxopiperidin-3-yl]-3-methyl- 2-oxo-2,3-dihydro-1H- benzimidazol-5- yl}propyl)piperazin-1-yl]pyrazin-2- yl}-2H-indazol-2- yl)cyclohexyl]methyl}-2,3,5- trifluoro-4-hydroxybenzamide, formate salt |
| 108 | 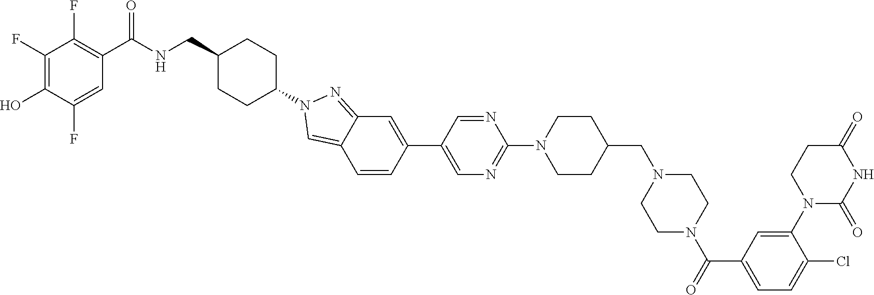 [SMILES: O=C1CCN(c2cc(C(=O)N3CCN(CC4CCN(c5ncc(-c6ccc7cn([C@H]8CC[C@H](CNC(=O)c9cc(F)c(O)c(F)c9F)CC8)nc7c6)cn5)CC4)CC3)ccc2Cl)C(=O)N1] | N-{[(1r,4r)-4-(6-{2-[4-({4-[4-chloro- 3-(2,4-dioxo-1,3-diazinan-1- yl)benzoyl]piperazin-1- yl}methyl)piperidin-1-yl]pyrimidin- 5-yl}-2H-indazol-2- yl)cyclohexyl]methyl}-2,3,5- trifluoro-4-hydroxybenzamide |
| 109 | 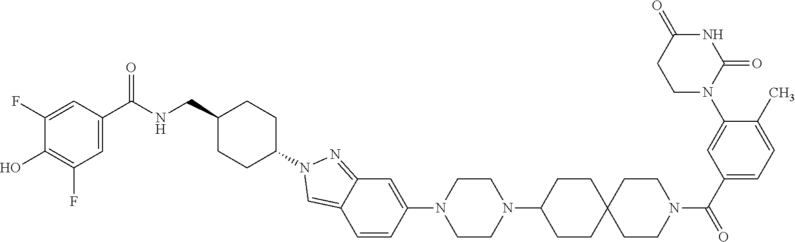 [SMILES: Cc1ccc(C(=O)N2CCC3(CCC(N4CCN(c5ccc6cn([C@H]7CC[C@H](CNC(=O)c8cc(F)c(O)c(F)c8)CC7)nc6c5)CC4)CC3)CC2)cc1N1CCC(=O)NC1=O] | N-({(1r,4r)-4-[6-(4-{3-[3-(2,4-dioxo- 1,3-diazinan-1-yl)-4- methylbenzoyl]-3- azaspiro[5.5]undecan-9- yl}piperazin-1-yl)-2H-indazol-2- yl]cyclohexyl}methyl)-3,5-difluoro- 4-hydroxybenzamide |
| 110 | 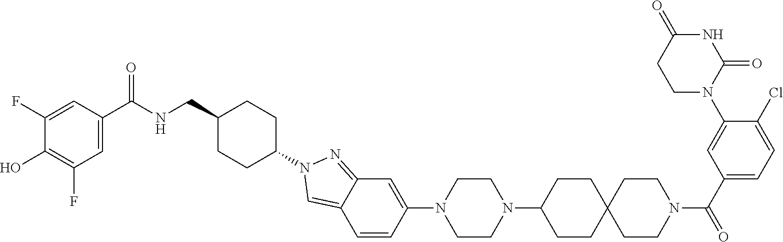 [SMILES: O=C1CCN(c2cc(C(=O)N3CCC4(CCC(N5CCN(c6ccc7cn([C@H]8CC[C@H](CNC(=O)c9cc(F)c(O)c(F)c9)CC8)nc7c6)CC5)CC4)CC3)ccc2Cl)C(=O)N1] | N-({(1r,4r)-4-[6-(4-{3-[4-chloro-3- (2,4-dioxo-1,3-diazinan-1- yl)benzoyl]-3- azaspiro[5.5]undecan-9- yl}piperazin-1-yl)-2H-indazol-2- yl]cyclohexyl}methyl)-3,5-difluoro- 4-hydroxybenzamide |
| 111 | 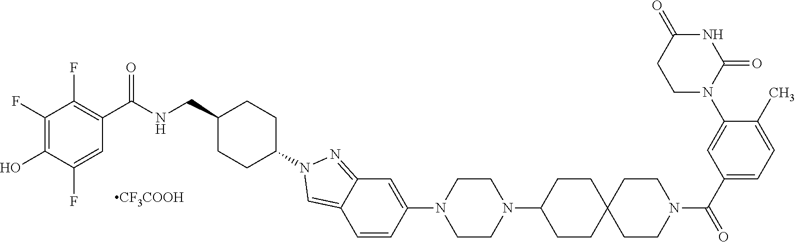 | N-({(1r,4r)-4-[6-(4-{3-[3-(2,4-dioxo- 1,3-diazinan-1-yl)-4- methylbenzoyl]-3- azaspiro[5.5]undecan-9- yl}piperazin-1-yl)-2H-indazol-2- yl]cyclohexyl}methyl)-2,3,5- trifluoro-4-hydroxybenzamide, trifluoroacetate salt |
| 112 | 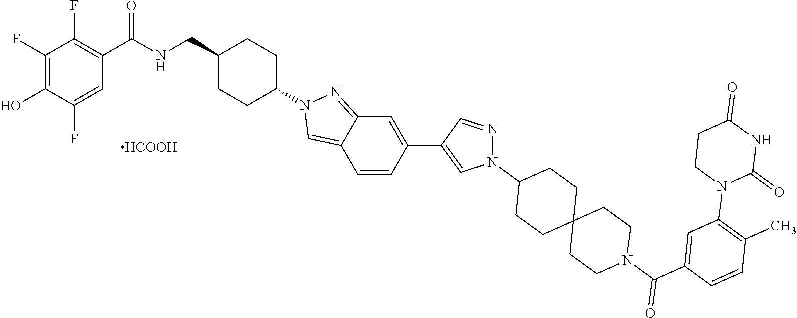 [SMILES: Cc1ccc(C(=O)N2CCC3(CCC(n4cc(-c5ccc6cn([C@H]7CC[C@H](CNC(=O)c8cc(F)c(O)c(F)c8F)CC7)nc6c5)cn4)CC3)CC2)cc1N1CCC(=O)NC1=O ; "·HCOOH"] | N-({(1r,4r)-4-[6-(1-{3-[3-(2,4-dioxo- 1,3-diazinan-1-yl)-4- methylbenzoyl]-3- azaspiro[5.5]undecan-9-yl}-1H- pyrazol-4-yl)-2H-indazol-2- yl]cyclohexyl}methyl)-2,3,5- trifluoro-4-hydroxybenzamide, formate salt |
| 113 | 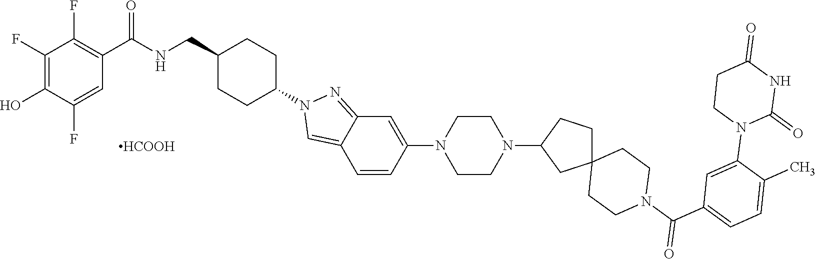 [SMILES: Cc1ccc(C(=O)N2CCC3(CCC(N4CCN(c5ccc6cn([C@H]7CC[C@H](CNC(=O)c8cc(F)c(O)c(F)c8F)CC7)nc6c5)CC4)C3)CC2)cc1N1CCC(=O)NC1=O ; "·HCOOH"] | N-({(1r,4r)-4-[6-(4-{8-[3-(2,4-dioxo- 1,3-diazinan-1-yl)-4- methylbenzoyl]-8- azaspiro[4.5]decan-2-yl}piperazin- 1-yl)-2H-indazol-2- yl]cyclohexyl}methyl)-2,3,5- trifluoro-4-hydroxybenzamide, formate salt |
| 114 | 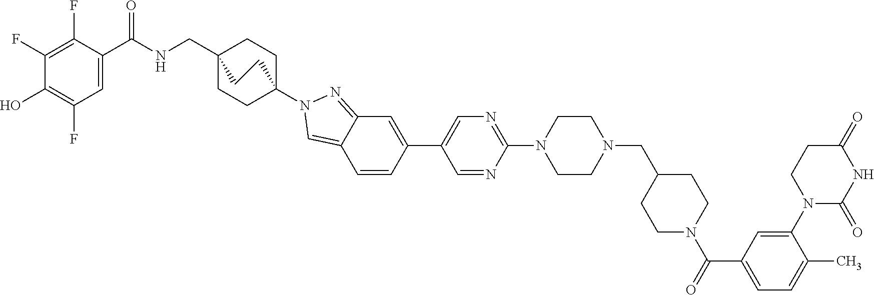 [SMILES: Cc1ccc(C(=O)N2CCC(CN3CCN(c4ncc(-c5ccc6cn([C@]78CC[C@](CNC(=O)c9cc(F)c(O)c(F)c9F)(CC7)CC8)nc6c5)cn4)CC3)CC2)cc1N1CCC(=O)NC1=O] | N-{[4-(6-{2-[4-({1-[3-(2,4-dioxo- 1,3-diazinan-1-yl)-4- methylbenzoyl]piperidin-4- yl}methyl)piperazin-1-yl]pyrimidin- 5-yl}-2H-indazol-2- yl)bicyclo[2.2.2]octan-1-yl]methyl}- 2,3,5-trifluoro-4- hydroxybenzamide |
| 115 | 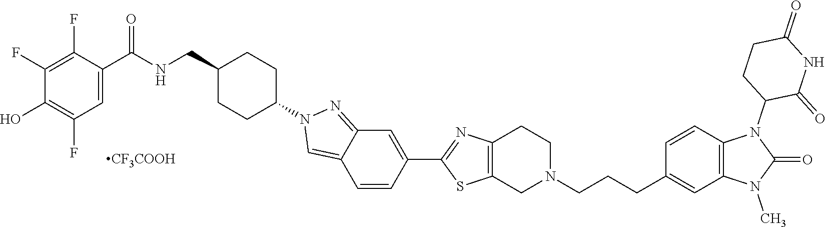 [SMILES: Cn1c(=O)n(C2CCC(=O)NC2=O)c2ccc(CCCN3CCc4nc(-c5ccc6cn([C@H]7CC[C@H](CNC(=O)c8cc(F)c(O)c(F)c8F)CC7)nc6c5)sc4C3)cc21 ; "·CF3COOH"] | N-{[(1r,4r)-4-{6-[5-(3-{1-[(3RS)- 2,6-dioxopiperidin-3-yl]-3-methyl- 2-oxo-2,3-dihydro-1H- benzimidazol-5-yl}propyl)-4,5,6,7- tetrahydro[1,3]thiazolo[5,4- c]pyridin-2-yl]-2H-indazol-2- yl}cyclohexyl]methyl}-2,3,5- trifluoro-4-hydroxybenzamide, trifluoroacetate salt |
| 116 | 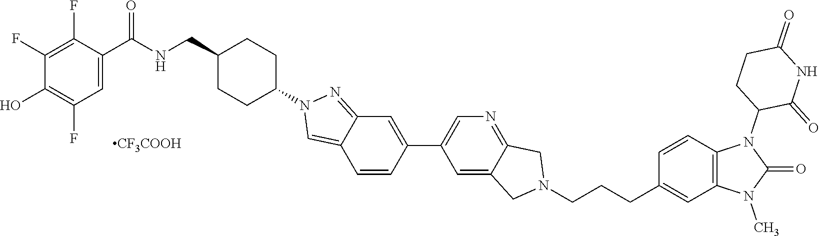 | N-{[(1r,4r)-4-{6-[6-(3-{1-[(3RS)- 2,6-dioxopiperidin-3-yl]-3-methyl- 2-oxo-2,3-dihydro-1H- benzimidazol-5-yl}propyl)-6,7- dihydro-5H-pyrrolo[3,4-b]pyridin- 3-yl]-2H-indazol-2- yl}cyclohexyl]methyl}-2,3,5- trifluoro-4-hydroxybenzamide, trifluoroacetate salt |
| 117 | 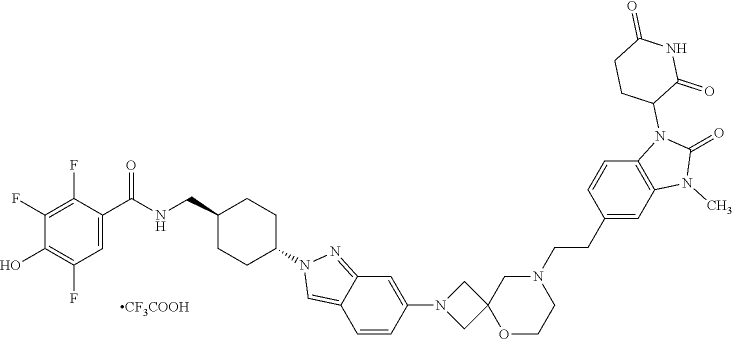 [SMILES: Cn1c(=O)n(C2CCC(=O)NC2=O)c2ccc(CCN3CCOC4(C3)CN(c3ccc5cn([C@H]6CC[C@H](CNC(=O)c7cc(F)c(O)c(F)c7F)CC6)nc5c3)C4)cc21 ; "·CF3COOH"] | N-{[(1r,4r)-4-{6-[8-(2-{1-[(3RS)- 2,6-dioxopiperidin-3-yl]-3-methyl- 2-oxo-2,3-dihydro-1H- benzimidazol-5-yl}ethyl)-5-oxa- 2,8-diazaspiro[3.5]nonan-2-yl]-2H- indazol-2-yl}cyclohexyl]methyl}- 2,3,5-trifluoro-4- hydroxybenzamide, trifluoroacetate salt |
| 118 | 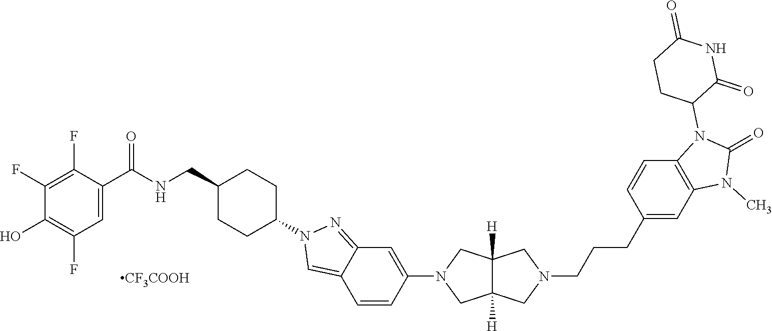 [SMILES: Cn1c(=O)n(C2CCC(=O)NC2=O)c2ccc(CCCN3C[C@H]4CN(c5ccc6cn([C@H]7CC[C@H](CNC(=O)c8cc(F)c(O)c(F)c8F)CC7)nc6c5)C[C@@H]4C3)cc21 ; "·CF3COOH"] | N-{[(1r,4r)-4-{6-[(3aS,6aS)-5-(3- {1-[(3RS)-2,6-dioxopiperidin-3-yl]- 3-methyl-2-oxo-2,3-dihydro-1H- benzimidazol-5- yl}propyl)hexahydropyrrolo[3,4- c]pyrrol-2(1H)-yl]-2H-indazol-2- yl}cyclohexyl]methyl}-2,3,5- trifluoro-4-hydroxybenzamide, trifluoroacetate salt |
| 119 | 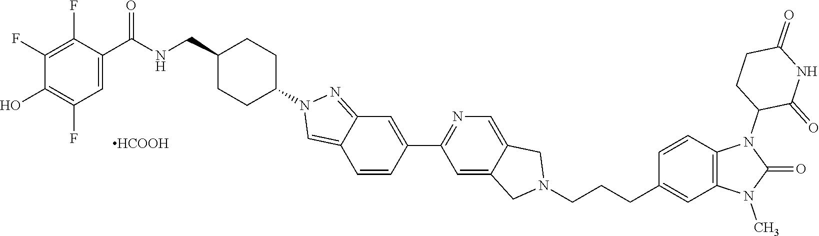 [SMILES: Cn1c(=O)n(C2CCC(=O)NC2=O)c2ccc(CCCN3Cc4cnc(-c5ccc6cn([C@H]7CC[C@H](CNC(=O)c8cc(F)c(O)c(F)c8F)CC7)nc6c5)cc4C3)cc21 ; "·HCOOH"] | N-{[(1r,4r)-4-{6-[2-(3-{1-[(3RS)- 2,6-dioxopiperidin-3-yl]-3-methyl- 2-oxo-2,3-dihydro-1H- benzimidazol-5-yl}propyl)-2,3- dihydro-1H-pyrrolo[3,4-c]pyridin-6- yl]-2H-indazol-2- yl}cyclohexyl]methyl}-2,3,5- trifluoro-4-hydroxybenzamide, formate salt |
| 120 | 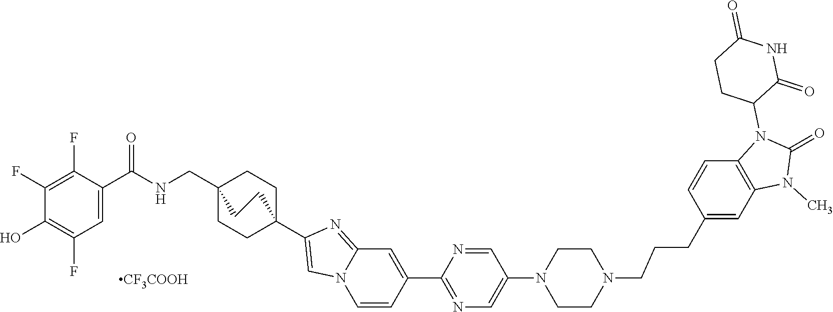 [SMILES: Cn1c(=O)n(C2CCC(=O)NC2=O)c2ccc(CCCN3CCN(c4cnc(-c5ccn6cc([C@]78CC[C@](CNC(=O)c9cc(F)c(O)c(F)c9F)(CC7)CC8)nc6c5)nc4)CC3)cc21 ; "·CF3COOH"] | N-{[4-(7-{5-[4-(3-{1-[(3RS)-2,6- dioxopiperidin-3-yl]-3-methyl-2- oxo-2,3-dihydro-1H-benzimidazol- 5-yl}propyl)piperazin-1- yl]pyrimidin-2-yl}imidazo[1,2- a]pyridin-2-yl)bicyclo[2.2.2]octan- 1-yl]methyl}-2,3,5-trifluoro-4- hydroxybenzamide, trifluoroacetate salt |
| 121 | 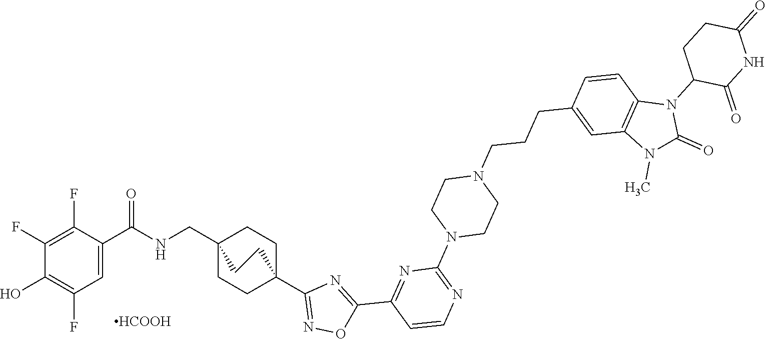 [SMILES: Cn1c(=O)n(C2CCC(=O)NC2=O)c2ccc(CCCN3CCN(c4nccc(-c5nc([C@]67CC[C@](CNC(=O)c8cc(F)c(O)c(F)c8F)(CC6)CC7)no5)n4)CC3)cc21 ; "·HCOOH"] | N-{[4-(5-{2-[4-(3-{1-[(3RS)-2,6- dioxopiperidin-3-yl]-3-methyl-2- oxo-2,3-dihydro-1H-benzimidazol- 5-yl}propyl)piperazin-1- yl]pyrimidin-4-yl}-1,2,4-oxadiazol- 3-yl)bicyclo[2.2.2]octan-1- yl]methyl}-3,5-difluoro-4- hydroxybenzamide, formate salt |
| 122 | 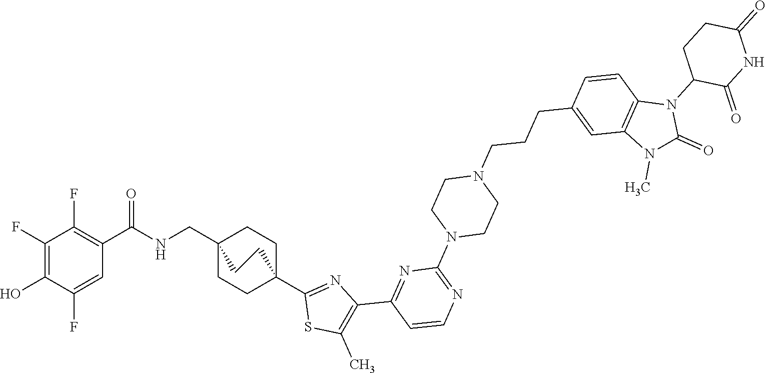 [SMILES: Cc1sc([C@]23CC[C@](CNC(=O)c4cc(F)c(O)c(F)c4F)(CC2)CC3)nc1-c1ccnc(N2CCN(CCCc3ccc4c(c3)n(C)c(=O)n4C3CCC(=O)NC3=O)CC2)n1] | N-{[4-(4-{2-[4-(3-{1-[(3RS)-2,6- dioxopiperidin-3-yl]-3-methyl-2- oxo-2,3-dihydro-1H-benzimidazol- 5-yl}propyl)piperazin-1- yl]pyrimidin-4-yl}-5-methyl-1,3- thiazol-2-yl)bicyclo[2.2.2]octan-1- yl]methyl}-2,3,5-trifluoro-4- hydroxybenzamide |
| 123 | 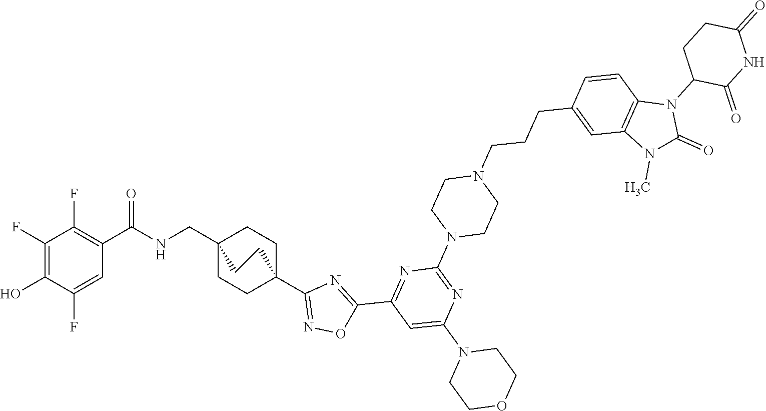 [SMILES: Cn1c(=O)n(C2CCC(=O)NC2=O)c2ccc(CCCN3CCN(c4nc(-c5nc([C@]67CC[C@](CNC(=O)c8cc(F)c(O)c(F)c8F)(CC6)CC7)no5)cc(N5CCOCC5)n4)CC3)cc21] | N-{[4-(5-{2-[4-(3-{1-[(3RS)-2,6- dioxopiperidin-3-yl]-3-methyl-2- oxo-2,3-dihydro-1H-benzimidazol- 5-yl}propyl)piperazin-1-yl]-6- (morpholin-4-yl)pyrimidin-4-yl}- 1,2,4-oxadiazol-3- yl)bicyclo[2.2.2]octan-1-yl]methyl}- 2,3,5-trifluoro-4- hydroxybenzamide |
| 124 | 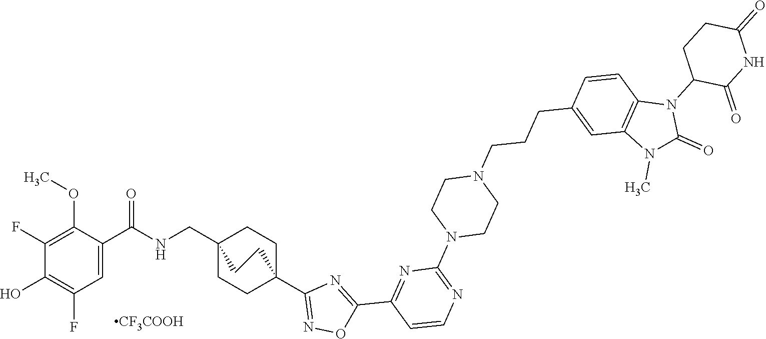 | N-{[4-(5-{2-[4-(3-{1-[(3RS)-2,6- dioxopiperidin-3-yl]-3-methyl-2- oxo-2,3-dihydro-1H-benzimidazol- 5-yl}propyl)piperazin-1- yl]pyrimidin-4-yl}-1,2,4-oxadiazol- 3-yl)bicyclo[2.2.2]octan-1- yl]methyl}-3,5-difluoro-4-hydroxy- 2-methoxybenzamide, trifluoroacetate salt |
| 125 | 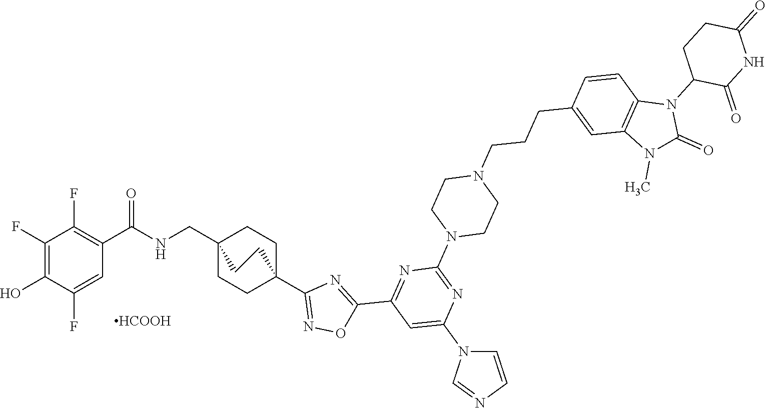 [SMILES: Cn1c(=O)n(C2CCC(=O)NC2=O)c2ccc(CCCN3CCN(c4nc(-c5nc([C@]67CC[C@](CNC(=O)c8cc(F)c(O)c(F)c8F)(CC6)CC7)no5)cc(-n5ccnc5)n4)CC3)cc21 ; "·HCOOH"] | N-{[4-(5-{2-[4-(3-{1-[(3RS)-2,6- dioxopiperidin-3-yl]-3-methyl-2- oxo-2,3-dihydro-1H-benzimidazol- 5-yl}propyl)piperazin-1-yl]-6-(1H- imidazol-1-yl)pyrimidin-4-yl}-1,2,4- oxadiazol-3-yl)bicyclo[2.2.2]octan- 1-yl]methyl}-2,3,5-trifluoro-4- hydroxybenzamide, formate salt |
| 126 | 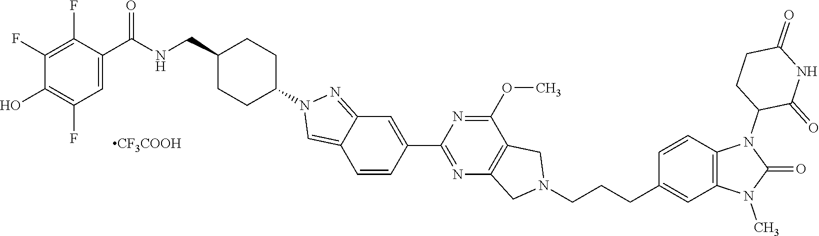 | N-{[(1r,4r)-4-{6-[6-(3-{1-[(3RS)- 2,6-dioxopiperidin-3-yl]-3-methyl- 2-oxo-2,3-dihydro-1H- benzimidazol-5-yl}propyl)-4- methoxy-6,7-dihydro-5H- pyrrolo[3,4-d]pyrimidin-2-yl]-2H- indazol-2-yl}cyclohexyl]methyl}- 2,3,5-trifluoro-4- hydroxybenzamide, trifluoroacetate salt |
| 127 | 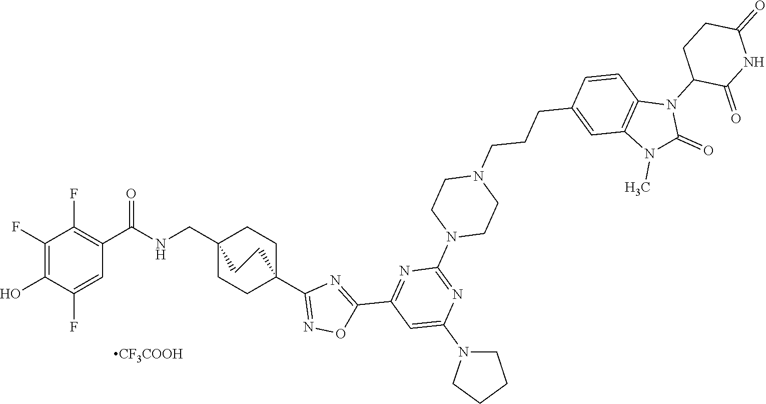 [SMILES: Cn1c(=O)n(C2CCC(=O)NC2=O)c2ccc(CCCN3CCN(c4nc(-c5nc([C@]67CC[C@](CNC(=O)c8cc(F)c(O)c(F)c8F)(CC6)CC7)no5)cc(N5CCCC5)n4)CC3)cc21 ; "·CF3COOH"] | N-{[4-(5-{2-[4-(3-{1-[(3RS)-2,6- dioxopiperidin-3-yl]-3-methyl-2- oxo-2,3-dihydro-1H-benzimidazol- 5-yl}propyl)piperazin-1-yl]-6- (pyrrolidin-1-yl)pyrimidin-4-yl}- 1,2,4-oxadiazol-3- yl)bicyclo[2.2.2]octan-1-yl]methyl}- 2,3,5-trifluoro-4- hydroxybenzamide, trifluoroacetate salt |
| 128 | 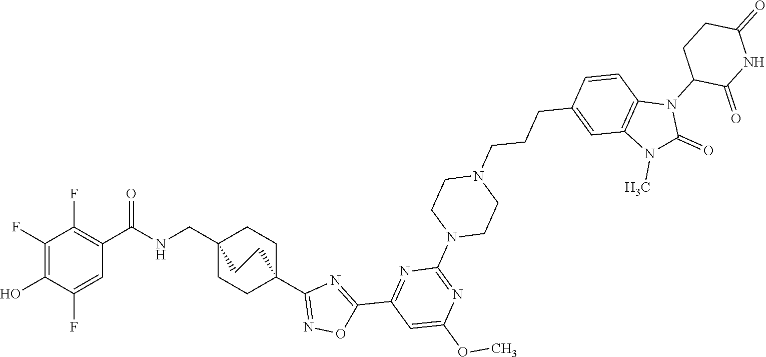 [SMILES: COc1cc(-c2nc([C@]34CC[C@](CNC(=O)c5cc(F)c(O)c(F)c5F)(CC3)CC4)no2)nc(N2CCN(CCCc3ccc4c(c3)n(C)c(=O)n4C3CCC(=O)NC3=O)CC2)n1] | N-{[4-(5-{2-[4-(3-{1-[(3RS)-2,6- dioxopiperidin-3-yl]-3-methyl-2- oxo-2,3-dihydro-1H-benzimidazol- 5-yl}propyl)piperazin-1-yl]-6- methoxypyrimidin-4-yl}-1,2,4- oxadiazol-3-yl)bicyclo[2.2.2]octan- 1-yl]methyl}-2,3,5-trifluoro-4- hydroxybenzamide |
| 129 | 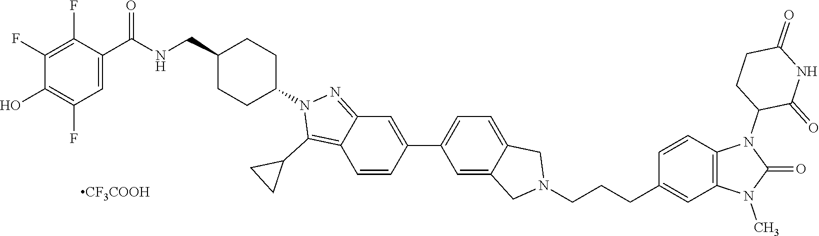 [SMILES: Cn1c(=O)n(C2CCC(=O)NC2=O)c2ccc(CCCN3Cc4ccc(-c5ccc6c(C7CC7)n([C@H]7CC[C@H](CNC(=O)c8cc(F)c(O)c(F)c8F)CC7)nc6c5)cc4C3)cc21 ; "·CF3COOH"] | N-{[(1r,4r)-4-{3-cyclopropyl-6-[2- (3-{1-[(3RS)-2,6-dioxopiperidin-3- yl]-3-methyl-2-oxo-2,3-dihydro- 1H-benzimidazol-5-yl}propyl)-2,3- dihydro-1H-isoindol-5-yl]-2H- indazol-2-yl}cyclohexyl]methyl}- 2,3,5-trifluoro-4- hydroxybenzamide, trifluoroacetate salt |
| 130 | 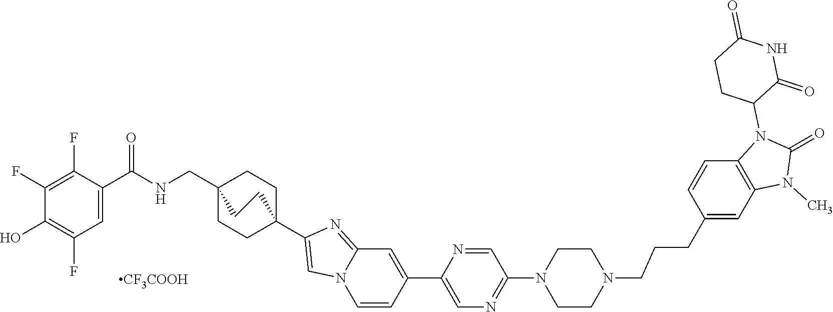 [SMILES: Cn1c(=O)n(C2CCC(=O)NC2=O)c2ccc(CCCN3CCN(c4cnc(-c5ccn6cc([C@]78CC[C@](CNC(=O)c9cc(F)c(O)c(F)c9F)(CC7)CC8)nc6c5)cn4)CC3)cc21 ; "·CF3COOH"] | N-{[4-(7-{5-[4-(3-{1-[(3RS)-2,6- dioxopiperidin-3-yl]-3-methyl-2- oxo-2,3-dihydro-1H-benzimidazol- 5-yl}propyl)piperazin-1-yl]pyrazin- 2-yl}imidazo[1,2-a]pyridin-2- yl)bicyclo[2.2.2]octan-1-yl]methyl}- 2,3,5-trifluoro-4- hydroxybenzamide, trifluoroacetate salt |
| 131 | 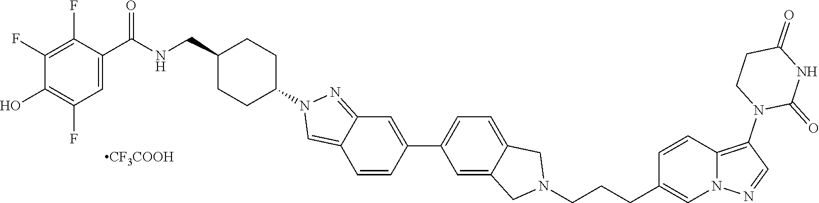 [SMILES: O=C1CCN(c2cnn3cc(CCCN4Cc5ccc(-c6ccc7cn([C@H]8CC[C@H](CNC(=O)c9cc(F)c(O)c(F)c9F)CC8)nc7c6)cc5C4)ccc23)C(=O)N1 ; "·CF3COOH"] | N-({(1r,4r)-4-[6-(2-{3-[3-(2,4-dioxo- 1,3-diazinan-1-yl)pyrazolo[1,5- a]pyridin-6-yl]propyl}-2,3-dihydro- 1H-isoindol-5-yl)-2H-indazol-2- yl]cyclohexyl}methyl)-2,3,5- trifluoro-4-hydroxybenzamide, trifluoroacetate salt |
| 132 | 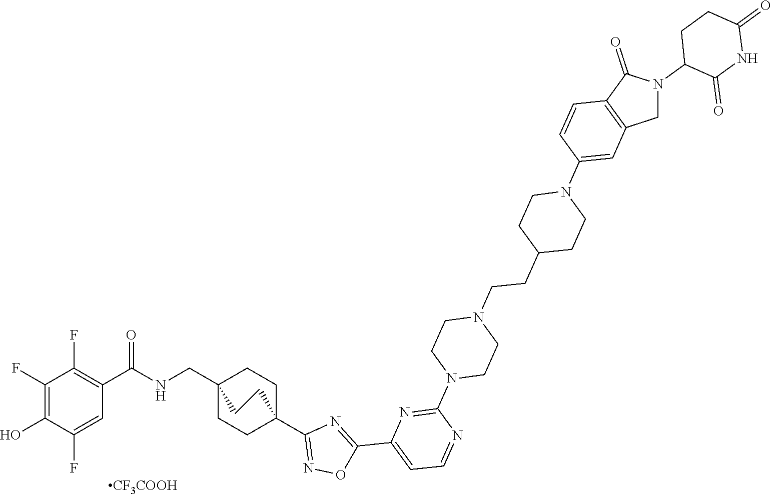 [SMILES: O=C1CCC(N2Cc3cc(N4CCC(CCN5CCN(c6nccc(-c7nc([C@]89CC[C@](CNC(=O)c%10cc(F)c(O)c(F)c%10F)(CC8)CC9)no7)n6)CC5)CC4)ccc3C2=O)C(=O)N1 ; "·CF3COOH"] | N-({4-[5-(2-{4-[2-(1-{2-[(3RS)-2,6- dioxopiperidin-3-yl]-1-oxo-2,3- dihydro-1H-isoindol-5-yl}piperidin- 4-yl)ethyl]piperazin-1-yl}pyrimidin- 4-yl)-1,2,4-oxadiazol-3- yl]bicyclo[2.2.2]octan-1-yl}methyl)- 2,3,5-trifluoro-4- hydroxybenzamide, trifluoroacetate salt |
| 133 | 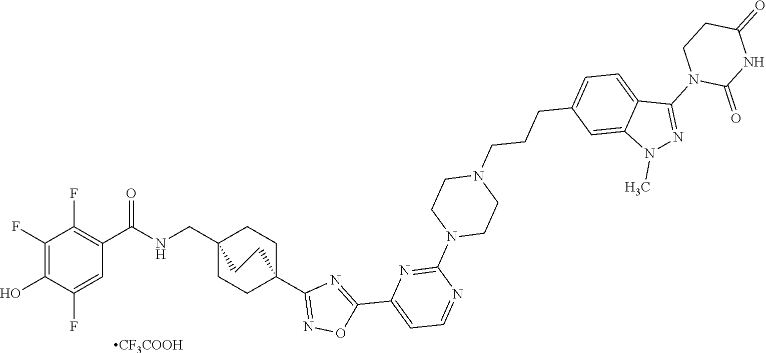 [SMILES: Cn1nc(N2CCC(=O)NC2=O)c2ccc(CCCN3CCN(c4nccc(-c5nc([C@]67CC[C@](CNC(=O)c8cc(F)c(O)c(F)c8F)(CC6)CC7)no5)n4)CC3)cc21 ; "·CF3COOH"] | N-[(4-{5-[2-(4-{3-[3-(2,4-dioxo-1,3- diazinan-1-yl)-1-methyl-1H- indazol-6-yl]propyl}piperazin-1- yl)pyrimidin-4-yl]-1,2,4-oxadiazol- 3-yl}bicyclo[2.2.2]octan-1- yl)methyl]-2,3,5-trifluoro-4- hydroxybenzamide, trifluoroacetate salt |
| 134 | 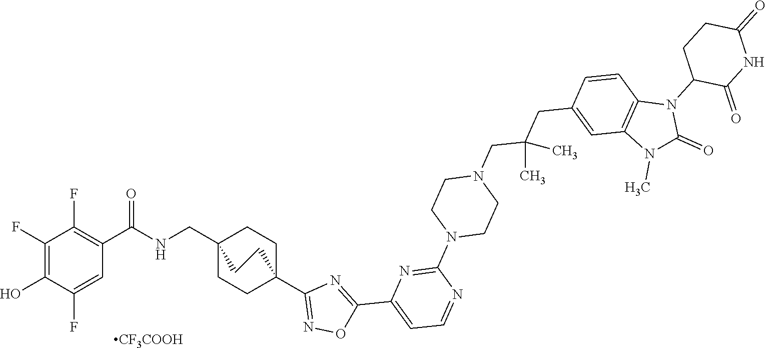 [SMILES: Cn1c(=O)n(C2CCC(=O)NC2=O)c2ccc(CC(C)(C)CN3CCN(c4nccc(-c5nc([C@]67CC[C@](CNC(=O)c8cc(F)c(O)c(F)c8F)(CC6)CC7)no5)n4)CC3)cc21 ; "·CF3COOH"] | N-{[4-(5-{2-[4-(3-{1-[(3RS)-2,6- dioxopiperidin-3-yl]-3-methyl-2- oxo-2,3-dihydro-1H-benzimidazol- 5-yl}-2,2-dimethylpropyl)piperazin- 1-yl]pyrimidin-4-yl}-1,2,4- oxadiazol-3-yl)bicyclo[2.2.2]octan- 1-yl]methyl}-2,3,5-trifluoro-4- hydroxybenzamide, trifluoroacetate salt |

TABLE 4
Method of synthesis and physicochemical data for Examples 23-134.

| | Method of | |
|---|---|---|
| | synthesis; | |
| | Non- | ¹H NMR (400 MHz, DMSO-d₆) δ; Mass spectrum, |
| | commercial | observed ion m/z [M + H]⁺ or HPLC retention time; |
| | starting | Mass spectrum m/z [M + H]⁺ (unless otherwise |
| Ex. | materials | indicated) |
| 23 | Example | ¹H NMR (400 MHz, methanol-d₄), characteristic peaks: δ |
| | 20¹; C20, | 8.25 (s, 1H), 8.07 (s, 1H), 7.90 (s, 1H), 7.72 (br s, 1H), |
| | P1, P239 | 7.70 (br d, J = 8.8 Hz, 1H), 7.53-7.43 (m, 2H), 7.34 (dd, |
| | | J = 8.7, 1.4 Hz, 1H), 7.33 (AB quartet, upfield doublet is |
| | | broadened, J_{AB} = 7.9 Hz, Δv_{AB} = 38.7 Hz, 2H), 7.32-7.30 |
| | | (m, 1H), 4.65-4.52 (m, 1H), 4.52-4.41 (m, 1H), 4.19 (t, |
| | | J = 6.9 Hz, 2H), 3.85 (ddd, half of ABXY system, J = 12.5, |
| | | 9.3, 5.7 Hz, 1H), 3.81-3.72 (m, 1H), 3.69-3.60 (m, |
| | | 1H), 3.15-3.01 (m, 1H), 2.93-2.74 (m, 3H), 2.32-2.23 |
| | | (m, 2H), 2.30 (s, 3H), 2.09-1.89 (m, 6H), 1.89-1.74 (m, |
| | | 2H), 1.73-1.52 (m, 2H), 1.25-1.04 (m, 2H); 807.5 |
| 24 | Example 19; | characteristic peaks: δ 11.38 (br s, 1H), 11.23 (br s, 1H), |
| | C176, P231 | 11.08 (s, 1H), 8.85 (s, 2H), 8.42 (s, 1H), 8.40-8.32 (m, |
| | | 1H), 7.89 (s, 1H), 7.77 (d, J = 8.6 Hz, 1H), 7.37-7.26 (m, |
| | | 2H), 7.11 (s, 1H), 6.99 (AB quartet, J_{AB} = 7.9 Hz, Δv_{AB} = |
| | | 49.9 Hz, 2H), 5.36 (dd, J = 12.6, 5.3 Hz, 1H), 4.79-4.68 |
| | | (m, 2H), 4.54-4.43 (m, 1H), 3.34 (s, 3H), 3.18 (br t, J = 6 |
| | | Hz, 2H), 3.14-2.98 (m, 4H), 2.97-2.84 (m, 1H), 2.78- |
| | | 2.56 (m, 4H), 2.22-2.05 (m, 4H), 2.05-1.83 (m, 5H), |
| | | 1.75-1.60 (m, 1H), 1.31-1.15 (m, 2H); 865.5 |
| 25 | Example 10; | 11.14 (s, 1H), 10.78 (br s, 1H), 8.87 (s, 2H), 8.43 (s, 1H), |
| | C176, P244 | 8.35 (br t, J = 6 Hz, 1H), 7.90 (s, 1H), 7.78 (d, J = 8.7 Hz, |
| | | 1H), 7.39-7.24 (m, 4H), 7.16 (t, J = 7.8 Hz, 1H), 5.46 |
| | | (dd, J = 12.6, 5.4 Hz, 1H), 4.80 (br d, J = 14 Hz, 2H), 4.67 |
| | | (br s, 2H), 4.55-4.43 (m, 1H), 3.67 (s, 3H), 3.6-3.42 |
| | | (m, 4H, assumed; largely obscured by water peak), 3.37- |
| | | 3.24 (m, 2H), 3.22-3.14 (m, 2H), 2.98-2.85 (m, 1H), |
| | | 2.81-2.59 (m, 2H), 2.22-2.11 (m, 2H), 2.06-1.98 (m, |
| | | 1H), 1.98-1.85 (m, 4H), 1.74-1.61 (m, 1H), 1.31-1.15 |
| | | (m, 2H); 837.7 |
| 26 | Example 19; | 11.28 (br s, 1H), 11.13 (s, 1H), 8.85 (s, 2H), 8.43 (s, 1H), |
| | C176, P245 | 8.36 (br t, J = 6 Hz, 1H), 7.89 (s, 1H), 7.78 (d, J = 8.7 Hz, |
| | | 1H), 7.54 (br s, 1H), 7.37-7.25 (m, 3H), 7.23 (d, half of |
| | | AB quartet, J = 8.1 Hz, 1H), 5.43 (dd, J = 12.8, 5.4 Hz, |
| | | 1H), 4.75 (d, J = 13.9 Hz, 2H), 4.54-4.43 (m, 1H), 4.41- |
| | | 4.34 (m, 2H), 3.55-3.38 (m, 4H), 3.37 (s, 3H), 3.18 (t, J = |
| | | 6.3 Hz, 2H), 3.14-3.01 (m, 2H), 2.98-2.85 (m, 1H), |
| | | 2.80-2.58 (m, 2H), 2.22-2.10 (m, 2H), 2.08-1.98 (m, |
| | | 1H), 1.98-1.84 (m, 4H), 1.74-1.60 (m, 1H), 1.30-1.15 |
| | | (m, 2H); 837.7 |
| 27 | Example 9; | characteristic peaks, integrations are approximate: δ |
| | C152, P231 | 11.37 (br s, 1H), 11.09 (s, 1H), 10.60 (br s, 1H), 8.38- |
| | | 8.30 (m, 1H), 8.26 (s, 1H), 7.57 (d, J = 9.1 Hz, 1H), 7.30 |
| | | (ddd, J = 11.1, 6.3, 2.3 Hz, 1H), 7.11 (br s, 1H), 7.06 (d, |
| | | half of AB quartet, J = 8.1 Hz, 1H), 6.98-6.88 (m, 3H), |
| | | 5.36 (dd, J = 12.7, 5.4 Hz, 1H), 4.45-4.33 (m, 1H), 3.35 |
| | | (s, 3H), 3.24-3.05 (m, 6H), 2.97-2.84 (m, 1H), 2.79- |
| | | 2.57 (m, 4H), 2.18-2.05 (m, 3H), 2.05-1.96 (m, 1H), |
| | | 1.96-1.79 (m, 4H), 1.72-1.58 (m, 1H), 1.28-1.13 (m, |
| | | 2H); 787.5 |
| 28 | Example | characteristic peaks; aliphatic integrations are |
| | 13^{2,3}; P229, | approximate: δ 10.43 (s, 1H), 8.46 (s, 1H), 8.30-8.22 |
| | P246 | (m, 1H), 8.15 (s, 1H), 7.96 (s, 1H), 7.52 (d, J = 9.1 Hz, |
| | | 1H), 7.46 (d, J = 9.2 Hz, 1H), 7.27 (ddd, J = 11.2, 6.4, 2.3 |
| | | Hz, 1H), 7.19 (br d, J = 9.1 Hz, 1H), 6.89 (br d, J = 9.1 |
| | | Hz, 1H), 6.78 (br s, 1H), 4.41-4.26 (m, 1H), 3.76 (t, J = |
| | | 6.7 Hz, 2H), 3.69-3.62 (m, 2H), 3.16 (br t, J = 6.4 Hz, |
| | | 2H), 3.03-2.93 (m, 2H), 2.76 (t, J = 6.7 Hz, 2H), 2.68- |
| | | 2.60 (m, 2H), 2.38-2.29 (m, 2H), 2.29-2.20 (m, 3H), |
| | | 2.15-2.05 (m, 2H), 1.98-1.72 (m, 6H), 1.72-1.59 (m, |
| | | 2H), 1.27-1.11 (m, 2H); 828.4 |
| 29 | Example | ¹H NMR (400 MHz, methanol-d₄), characteristic peaks, |
| | 28⁴; P229 | integrations are approximate: δ 8.37 (s, 1H), 8.34-8.27 |
| | | (m, <1H), 8.15 (s, 1H), 7.99 (s, 1H), 7.57 (d, J = 9.0 Hz, |
| | | 1H), 7.56 (br d, J = 9.1 Hz, 1H), 7.28 (ddd, J = 11.1, 6.2, |
| | | 2.3 Hz, 1H), 7.24 (dd, J = 9.2, 1.3 Hz, 1H), 6.55 (d, J = |
| | | 9.0 Hz, 1H), 6.46-6.44 (m, 1H), 4.43-4.32 (m, 1H), |
| | | 4.14 (d, J = 7.9 Hz, 2H), 3.91-3.83 (m, 4H), 3.70-3.54 |
| | | (m, 4H), 3.08-2.95 (m, 2H), 2.87 (t, J = 6.8 Hz, 2H), |
| | | 2.77 (br t, J = 7.4 Hz, 2H), 2.30-2.20 (m, 2H), 2.08- |
| | | 1.90 (m, 7H), 1.84-1.72 (m, 1H), 1.38-1.24 (m, 2H); 821.1 |
| 30 | Example | characteristic peaks, aliphatic integrations are |
| | 28^{5,3}; P229, | approximate: δ 11.42 (br s, 1H), 10.45 (s, 1H), 9.71 (br s, |
| | P246 | 1H), 8.57 (s, 1H), 8.38-8.31 (m, 1H), 8.17 (br s, 1H), |
| | | 7.99 (s, 1H), 7.58 (d, J = 9.1 Hz, 1H), 7.54-7.48 (m, 1H), |
| | | 7.29 (ddd, J = 11.0, 6.3, 2.3 Hz, 1H), 7.19 (d, J = 9.2 Hz, |
| | | 1H), 6.63 (d, J = 9.2 Hz, 1H), 6.37 (br s, 1H), 4.38-4.26 |
| | | (m, 1H), 3.76 (t, J = 6.7 Hz, 2H), 3.25-3.10 (m, 5H), |
| | | 2.77 (t, J = 6.7 Hz, 2H), 2.73-2.65 (m, 2H), 2.20-1.99 |
| | | (m, 5H), 1.95-1.76 (m, 4H), 1.71-1.57 (m, 1H), 1.27- |
| | | 1.11 (m, 2H); 813.8 |
| 31 | Example | 11.43 (br s, 1H), 11.09 (s, 1H), 8.52 (s, 1H), 8.35 (br t, J = |
| | 13⁶; P230, | 6 Hz, 1H), 7.90 (br s, 1H), 7.85 (d, J = 8.7 Hz, 1H), 7.81- |
| | P231 | 7.77 (m, 1H), 7.30 (ddd, J = 11.2, 6.3, 2.4 Hz, 1H), 7.21 |
| | | (dd, J = 8.6, 1.5 Hz, 1H), 7.09 (br s, 1H), 7.03 (d, half of |
| | | AB quartet, J = 8.0 Hz, 1H), 6.93 (dd, component of ABX |
| | | system, J = 8.1, 1.5 Hz, 1H), 5.35 (dd, J = 12.7, 5.4 Hz, |
| | | 1H), 4.58-4.47 (m, 1H), 4.32-4.23 (m, 2H), 4.18-4.07 |
| | | (m, 2H), 3.34 (s, 3H), 3.22-3.07 (m, 4H), 2.97-2.84 (m, |
| | | 1H), 2.82-2.57 (m, 6H), 2.22-2.11 (m, 2H), 2.05-1.84 |
| | | (m, 7H), 1.74-1.61 (m, 1H), 1.31-1.16 (m, 2H); 824.0 |
| 32 | Example | characteristic peaks: δ 11.10 (s, 1H), 8.42 (s, 1H), 8.36- |
| | 22⁷; P230, | 8.28 (m, 1H), 7.80 (br s, 1H), 7.76 (d, J = 8.8 Hz, 1H), |
| | P231 | 7.55 (br d, J = 8.0 Hz, 1H), 7.49 (br s, 1H), 7.35-7.22 |
| | | (m, 3H), 7.10 (br s, 1H), 6.98 (AB quartet, upfield doublet |
| | | is broadened, J_{AB} = 8.0 Hz, Δv_{AB} = 42.5 Hz, 2H), 5.35 (dd, |
| | | J = 12.9, 5.4 Hz, 1H), 4.55-4.41 (m, 1H), 4.13-3.88 |
| | | (m, 1H), 3.18 (t, J = 6.3 Hz, 2H), 3.02-2.92 (m, 2H), |
| | | 2.93-2.83 (m, 1H), 2.22-2.11 (m, 2H), 2.05-1.85 (m, |
| | | 7H), 1.74-1.60 (m, 1H); 833.8 |
| 33 | Example 32; | characteristic peaks, integrations are approximate: δ |
| | P230, P231 | 11.40 (br s, 1H), 11.10 (s, 1H), 10.53 (br s, 1H), 8.36 (s, |
| | | 1H), 8.23-8.16 (m, 1H), 7.85-7.81 (m, 1H), 7.69 (d, J = |
| | | 8.7 Hz, 1H), 7.34-7.26 (m, 2H), 7.12-7.03 (m, 2H), |
| | | 6.96-6.90 (m, 1H), 5.43-5.28 (m, 2H), 4.74-4.61 (m, |
| | | 1H), 4.61-4.33 (m, 3H), 3.17 (t, J = 6.3 Hz, 2H), 2.98- |
| | | 2.84 (m, 1H), 2.78-2.57 (m, 4H), 2.21-2.10 (m, 2H), |
| | | 2.06-1.81 (m, 7H), 1.75-1.60 (m, 1H); 824.0 |
| 34 | Example 31; | characteristic peaks, integrations are approximate: δ |
| | P230, P231 | 11.41 (br s, 1H), 11.11 (s, 1H), 9.82 (br s, 1H), 8.43 (s, |
| | | 1H), 8.35 (br t, J = 6 Hz, 1H), 7.86 (s, 1H), 7.77 (d, J = |
| | | 8.7 Hz, 1H), 7.66-7.61 (m, 2H), 7.35 (dd, J = 8.8, 1.5 |
| | | Hz, 1H), 7.33-7.26 (m, 1H), 7.29 (d, J = 8.8 Hz, 1H), |
| | | 7.11 (br s, 1H), 5.36 (dd, J = 12.7, 5.4 Hz, 1H), 4.66- |
| | | 4.57 (m, 1H), 4.54-4.43 (m, 1H), 4.40-4.30 (m, 1H), |
| | | 3.85-3.72 (m, 1H), 3.22-3.13 (m, 4H), 2.79-2.57 (m, |
| | | 3H), 2.22-2.05 (m, 3H), 2.05-1.83 (m, 4H), 1.74-1.60 |
| | | (m, 1H), 1.31-1.15 (m, 2H); 834.0 |
| 35 | Example | ¹H NMR (600 MHz, DMSO-d₆), characteristic peaks, |
| | 22⁸; C144, | integrations are approximate: δ 11.48 (br s, 1H), 10.98 (s, |
| | P238 | 1H), 9.40 (br s, 1H), 8.96 (s, 2H), 8.45 (s, 1H), 8.38- |
| | | 8.32 (m, 1H), 7.95 (br s, 1H), 7.81 (d, J = 8.7 Hz, 1H), |
| | | 7.55 (AB quartet, J_{AB} = 7.7 Hz, Δv_{AB} = 38.1 Hz, 4H), 7.49 |
| | | (t, J = 7.9 Hz, 1H), 7.37 (d, J = 8.8 Hz, 1H), 7.34 (d, J = |
| | | 7.6 Hz, 1H), 7.33-7.27 (m, 1H), 7.32 (d, J = 8.6 Hz, 1H), |
| | | 5.30 (s, 2H), 5.11 (ddd, J = 13.5, 5.3, 2.7 Hz, 1H), 4.53- |
| | | 4.46 (m, 1H), 4.46-4.37 (m, 3H), 4.31-4.25 (m, 2H), |
| | | 3.18 (t, J = 6.3 Hz, 2H), 3.01-2.85 (m, 3H), 2.63-2.55 |
| | | (m, 1H), 2.47-2.37 (m, 1H), 2.20-2.12 (m, 2H), 2.04- |
| | | 1.86 (m, 7H), 1.79-1.62 (m, 4H), 1.48-1.36 (m, 2H), |
| | | 1.28-1.18 (m, 2H); 972.0 |
| 36 | Example | ¹H NMR (600 MHz, DMSO-d₆) δ 11.45 (br s, 1H), 10.98 |
| | 15^{9,10}; P9, | (s, 1H), 9.31 (br s, 1H), 8.21 (br t, J = 6 Hz, 1H), 8.12 (d, |
| | P238 | J = 9.2 Hz, 1H), 7.55 (AB quartet, J_{AB} = 7.9 Hz, Δv_{AB} = |
| | | 41.2 Hz, 4H), 7.49 (t, J = 7.8 Hz, 1H), 7.36 (d, J = 9.2 Hz, |
| | | 1H), 7.34 (d, J = 7.5 Hz, 1H), 7.32 (d, J = 8.2 Hz, 1H), |
| | | 7.30-7.25 (m, 1H), 5.30 (s, 2H), 5.14-5.08 (m, 1H), |
| | | 4.51 (t, J = 6.6 Hz, 2H), 4.43 (d, half of AB quartet, J = |
| | | 17.4 Hz, 1H), 4.31-4.25 (m, 3H), 3.08 (d, J = 6.3 Hz, |
| | | 2H), 2.96-2.86 (m, 3H), 2.62-2.56 (m, 1H), 2.47-2.37 |
| | | (m, 1H), 2.03-1.94 (m, 8H), 1.93-1.86 (m, 2H), 1.85- |
| | | 1.77 (m, 2H), 1.60-1.49 (m, 8H), 1.41-1.26 (m, 4H); 963.8 |
| 37 | Example | ¹H NMR (600 MHz, DMSO-d₆) δ 10.98 (s, 1H), 10.89 (br |
| | 36^{9,11,12}; P1, | s, 1H), 9.36 (br s, 1H), 8.31 (t, J = 6.3 Hz, 1H), 8.12 (d, J = |
| | P238 | 9.2 Hz, 1H), 7.61-7.56 (m, 4H), 7.52 (d, half of AB |
| | | quartet, J = 7.9 Hz, 2H), 7.49 (t, J = 7.8 Hz, 1H), 7.36 (d, |
| | | J = 9.2 Hz, 1H), 7.34 (d, J = 7.6 Hz, 1H), 7.32 (d, J = 8.3 |
| | | Hz, 1H), 5.30 (s, 2H), 5.11 (dd, J = 13.3, 5.1 Hz, 1H), |
| | | 4.51 (t, J = 6.6 Hz, 2H), 4.43 (d, J = 17.4 Hz, 1H), 4.31- |
| | | 4.25 (m, 3H), 3.10 (d, J = 6.3 Hz, 2H), 2.96-2.86 (m, |
| | | 3H), 2.62-2.55 (m, 1H), 2.48-2.38 (m, 1H), 2.03-1.93 |
| | | (m, 8H), 1.93-1.86 (m, 2H), 1.85-1.77 (m, 2H), 1.59- |
| | | 1.49 (m, 8H), 1.41-1.27 (m, 4H); 945.8 |
| 38 | C66, C144¹³ | ¹H NMR (600 MHz, DMSO-d₆), characteristic peaks: δ |
| | | 11.41 (br s, 1H), 11.07 (s, 1H), 8.66 (s, 2H), 8.39 (s, 1H), |
| | | 8.36-8.31 (m, 1H), 7.80 (s, 1H), 7.74 (d, J = 8.7 Hz, 1H), |
| | | 7.36 (br s, 1H), 7.32-7.27 (m, 2H), 7.04 (s, 1H), 6.94 |
| | | (AB quartet, J_{AB} = 8.0 Hz, Δv_{AB} = 68.5 Hz, 2H), 5.32 (dd, J = |
| | | 12.9, 5.4 Hz, 1H), 4.52-4.43 (m, 1H), 3.31 (s, 3H), |
| | | 3.18 (t, J = 6.3 Hz, 2H), 2.93-2.84 (m, 1H), 2.73-2.57 |
| | | (m, 4H), 2.20-2.12 (m, 2H), 2.02-1.96 (m, 1H), 1.96- |
| | | 1.86 (m, 4H), 1.72-1.62 (m, 3H), 1.62-1.55 (m, 2H), |
| | | 1.28-1.17 (m, 2H); 810.7 |
| 39 | C66, | ¹H NMR (600 MHz, DMSO-d₆) δ 11.41 (br s, 1H), 11.07 |
| | C152^{14,15} | (s, 1H), 8.35-8.30 (m, 1H), 8.20 (s, 1H), 7.52 (d, J = 9.1 |
| | | Hz, 1H), 7.29 (ddd, J = 11.1, 6.3, 2.2 Hz, 1H), 7.03 (s, |
| | | 1H), 6.98 (d, J = 8.0 Hz, 1H), 6.93 (br d, J = 9.2 Hz, 1H), |
| | | 6.86 (br d, J = 8.0 Hz, 1H), 6.82 (br s, 1H), 5.32 (dd, J = |
| | | 12.9, 5.4 Hz, 1H), 4.39-4.32 (m, 1H), 3.65-3.56 (m, |
| | | 4H), 3.31 (s, 3H), 3.16 (t, J = 6.3 Hz, 2H), 3.13-3.07 (m, |
| | | 2H), 3.07-3.02 (m, 2H), 2.93-2.84 (m, 1H), 2.73-2.64 |
| | | (m, 1H), 2.64-2.57 (m, 3H), 2.35 (t, J = 7.4 Hz, 2H), |
| | | 2.14-2.07 (m, 2H), 2.02-1.96 (m, 1H), 1.93-1.81 (m, |
| | | 4H), 1.69-1.51 (m, 5H), 1.37-1.29 (m, 2H), 1.25-1.15 |
| | | (m, 2H); 843.9 |
| 40 | Example | ¹H NMR (600 MHz, DMSO-d₆), characteristic peaks, |
| | 14¹⁶; P239, | aliphatic integrations are approximate: δ 11.45 (br s, 1H), |
| | C152 | 10.38 (s, 1H), 9.55 (br s, 1H), 8.37-8.30 (m, 1H), 8.23 |
| | | (s, 1H), 7.56 (d, J = 9.0 Hz, 1H), 7.35 (d, half of AB |
| | | quartet, J = 7.8 Hz, 1H), 7.32-7.27 (m, 2H), 7.25 (dd, |
| | | component of ABX system, J = 7.7, 1.8 Hz, 1H), 6.96- |
| | | 6.90 (m, 2H), 4.42-4.32 (m, 1H), 3.87-3.74 (m, 3H), |
| | | 3.20-3.10 (m, 5H), 2.83-2.75 (m, 1H), 2.73-2.66 (m, |
| | | 1H), 2.22 (s, 3H), 2.15-2.07 (m, 2H), 1.95-1.60 (m, |
| | | 10H), 1.60-1.49 (m, 2H), 1.31-1.25 (m, 2H), 1.25- |
| | | 1.15 (m, 2H), 1.15-1.04 (m, 2H); 844.0 |
| 41 | C66, | ¹H NMR (600 MHz, DMSO-d₆), characteristic peaks, |
| | C144^{17,18} | aliphatic integrations are approximate: δ 11.39 (br s, 1H), |
| | | 11.08 (s, 1H), 8.72 (s, 2H), 8.40 (s, 1H), 8.33 (br t, J = 6 |
| | | Hz, 1H), 7.82 (br s, 1H), 7.75 (d, J = 8.7 Hz, 1H), 7.32- |
| | | 7.27 (m, 2H), 7.09 (br s, 1H), 6.96 (AB quartet, J_{AB} = 8.1 |
| | | Hz, Δv_{AB} = 64.6 Hz, 2H), 5.34 (dd, J = 13.0, 5.5 Hz, 1H), |
| | | 4.51-4.43 (m, 1H), 4.16 (t, J = 8.3 Hz, 2H), 3.72 (dd, J = |
| | | 8.7, 5.6 Hz, 2H), 3.18 (t, J = 6.3 Hz, 2H), 2.90 (ddd, J = |
| | | 17.0, 13.5, 5.4 Hz, 1H), 2.75-2.68 (m, 2H), 2.68-2.58 |
| | | (m, 3H), 2.20-2.12 (m, 2H), 2.04-1.86 (m, 6H), 1.72- |
| | | 1.62 (m, 1H), 1.28-1.17 (m, 2H); 822.5 |
| 42 | Example | ¹H NMR (600 MHz, DMSO-d₆), characteristic peaks, |
| | 20¹⁹; C144, | aliphatic integrations are approximate: δ 11.45 (br s, 1H), |
| | P239 | 10.41 (s, 1H), 9.52 (br s, 1H), 8.76 (s, 2H), 8.40 (s, 1H), |
| | | 8.34 (br t, J = 6 Hz, 1H), 7.84 (s, 1H), 7.76 (d, J = 8.7 Hz, |
| | | 1H), 7.42-7.38 (m, 2H), 7.36 (br d, half of AB quartet, J = |
| | | 7.8 Hz, 1H), 7.33-7.27 (m, 2H), 4.76-4.68 (m, 2H), |
| | | 4.51-4.43 (m, 1H), 3.84-3.75 (m, 1H), 3.58-3.51 (m, |
| | | 1H), 3.18 (t, J = 6.4 Hz, 2H), 3.02-2.94 (m, 2H), 2.81 |
| | | (ddd, component of ABXY system, J = 16.2, 10.0, 6.2 Hz, |
| | | 1H), 2.69 (ddd, component of ABXY system, J = 16.5, |
| | | 5.4, 5.4 Hz, 1H), 2.24 (s, 3H), 2.20-2.11 (m, 3H), 1.96- |
| | | 1.86 (m, 4H), 1.86-1.79 (m, 2H), 1.71-1.62 (m, 1H), |
| | | 1.28-1.15 (m, 4H); 894.0 |
| 43 | Example 19; | ¹H NMR (600 MHz, DMSO-d₆), characteristic peaks, |
| | C176, P240 | aliphatic integrations are approximate: δ 11.46 (br s, 1H), |
| | | 11.10 (s, 1H), 10.05 (br s, 1H), 8.87 (s, 2H), 8.42 (s, 1H), |
| | | 8.38-8.31 (m, 1H), 7.90 (s, 1H), 7.79 (d, J = 8.7 Hz, 1H), |
| | | 7.35 (br d, J = 8.6 Hz, 1H), 7.30 (ddd, J = 11.0, 6.1, 2.2 |
| | | Hz, 1H), 7.14 (br s, 1H), 7.10 (d, J = 7.8 Hz, 1H), 6.98 (br |
| | | d, J = 8.1 Hz, 1H), 5.36 (dd, J = 13.0, 5.4 Hz, 1H), 4.86- |
| | | 4.70 (m, 2H), 4.53-4.44 (m, 1H), 3.76-3.62 (m, 2H), |
| | | 3.34 (s, 3H), 3.21-3.14 (m, 3H), 3.11-3.04 (m, 2H), |
| | | 2.90 (ddd, J = 17.4, 13.6, 5.4 Hz, 1H), 2.76-2.66 (m, |
| | | 1H), 2.65-2.60 (m, 1H), 2.20-2.13 (m, 2H), 2.04-1.97 |
| | | (m, 1H), 1.97-1.86 (m, 4H), 1.72-1.63 (m, 1H), 1.28- |
| | | 1.18 (m, 2H); 852.0 |
| 44 | Example 22²⁰; | ¹H NMR (600 MHz, DMSO-d₆), characteristic peaks, |
| | P229, | aliphatic integrations are approximate: δ 11.44 (br s, 1H), |
| | P240 | 11.08 (s, 1H), 9.17 (br s, 1H), 8.37 (s, 1H), 8.36-8.32 |
| | | (m, 1H), 7.64 (d, J = 8.5 Hz, 1H), 7.48 (br s, 1H), 7.29 |
| | | (ddd, J = 11.1, 6.2, 2.2 Hz, 1H), 7.09 (br s, 1H), 7.06 (d, J = |
| | | 8.1 Hz, 1H), 6.95-6.91 (m, 2H), 5.34 (dd, J = 13.0, 5.4 |
| | | Hz, 1H), 4.50-4.39 (m, 1H), 3.55-3.48 (m, 2H), 3.32 |
| | | (s, 3H), 3.20-3.14 (m, 2H), 3.14-3.04 (m, 2H), 3.01- |
| | | 2.94 (m, 1H), 2.94-2.84 (m, 1H), 2.73-2.66 (m, 1H), |
| | | 2.64-2.58 (m, 1H), 2.17-2.09 (m, 2H), 2.06-1.95 (m, |
| | | 3H), 1.95-1.82 (m, 5H), 1.71-1.61 (m, 1H), 1.27-1.16 |
| | | (m, 2H); 804.6 |
| 45 | Example 42; | ¹H NMR (600 MHz, DMSO-d₆), characteristic peaks, |
| | C144, P239 | aliphatic integrations are approximate: δ 11.43 (br s, 1H), |
| | | 10.39 (s, 1H), 9.52 (br s, 1H), 8.86 (s, 2H), 8.42 (s, 1H), |
| | | 8.34 (br t, J = 6 Hz, 1H), 7.90 (s, 1H), 7.78 (d, J = 8.7 Hz, |
| | | 1H), 7.36 (d, J = 7.9 Hz, 1H), 7.34 (d, J = 8.8 Hz, 1H), |
| | | 7.32 (br s, 1H), 7.32-7.28 (m, 1H), 7.26 (br d, J = 7.8 |
| | | Hz, 1H), 4.85-4.63 (m, 2H), 4.54-4.43 (m, 2H), 3.83- |
| | | 3.75 (m, 1H), 3.72-3.50 (m, 3H), 3.18 (t, J = 6.4 Hz, |
| | | 2H), 3.15-3.01 (m, 4H), 2.85-2.75 (m, 1H), 2.73-2.65 |
| | | (m, 1H), 2.22 (s, 3H), 2.20-2.11 (m, 3H), 1.97-1.87 (m, |
| | | 4H), 1.73-1.62 (m, 1H), 1.28-1.17 (m, 4H); 893.6 |
| 46 | Example 41; | ¹H NMR (600 MHz, DMSO-d₆), characteristic peaks: δ |
| | C66, C144 | 11.39 (br s, 1H), 11.08 (s, 1H), 8.73 (br s, 2H), 8.40 (s, |
| | | 1H), 8.34 (br t, J = 6 Hz, 1H), 7.82 (s, 1H), 7.75 (d, J = |
| | | 8.7 Hz, 1H), 7.33-7.27 (m, 2H), 7.04 (s, 1H), 7.02 (d, J = |
| | | 8.0 Hz, 1H), 6.87 (d, J = 8.0 Hz, 1H), 5.34 (dd, J = 13.0, |
| | | 5.4 Hz, 1H), 4.74-4.66 (m, 2H), 4.51-4.43 (m, 1H), |
| | | 3.33 (s, 3H), 3.18 (t, J = 6.3 Hz, 2H), 2.92-2.83 (m, 2H), |
| | | 2.75-2.66 (m, 1H), 2.65-2.59 (m, 1H), 2.59-2.56 (m, |
| | | 2H), 2.20-2.12 (m, 2H), 2.05-1.97 (m, 1H), 1.96-1.81 |
| | | (m, 5H), 1.73-1.62 (m, 3H), 1.28-1.10 (m, 4H); 836.5 |
| 47 | Example | ¹H NMR (600 MHz, DMSO-d₆), characteristic peaks: δ |
| | 10²¹; C152, | 10.85 (s, 1H), 8.69 (d, J = 6.9 Hz, 1H), 8.36-8.31 (m, |
| | P242 | 1H), 8.23 (s, 1H), 7.56 (d, J = 9.0 Hz, 1H), 7.31-7.27 (m, |
| | | 1H), 4.41-4.33 (m, 1H), 2.92-2.81 (m, 4H), 2.16-2.06 |
| | | (m, 4H), 1.94-1.80 (m, 4H), 1.69-1.60 (m, 1H), 1.25- |
| | | 1.15 (m, 2H); 758.9 |
| 48 | Example 17; | ¹H NMR (600 MHz, DMSO-d₆) δ 11.46 (br s, 1H), 10.43 |
| | C152, P241 | (s, 1H), 9.62 (br s, 1H), 8.57 (s, 1H), 8.33 (br t, J = 6 Hz, |
| | | 1H), 8.23 (s, 1H), 7.99 (s, 1H), 7.58 (d, J = 9.3 Hz, 1H), |
| | | 7.56 (d, J = 9.2 Hz, 1H), 7.29 (ddd, J = 11.2, 6.2, 2.2 Hz, |
| | | 1H), 7.21 (br d, J = 9.1 Hz, 1H), 6.94 (br d, J = 9.2 Hz, |
| | | 1H), 6.92 (br s, 1H), 4.42-4.32 (m, 1H), 3.86-3.76 (m, |
| | | 2H), 3.77 (t, J = 6.7 Hz, 2H), 3.68-3.58 (m, 2H), 3.26- |
| | | 3.12 (m, 6H), 3.03-2.90 (m, 2H), 2.78 (t, J = 6.7 Hz, |
| | | 2H), 2.71 (br t, J = 7.4 Hz, 2H), 2.15-2.03 (m, 4H), 1.94- |
| | | 1.80 (m, 4H), 1.70-1.59 (m, 1H), 1.26-1.15 (m, 2H); |
| | | 758.4 |
| 49 | P239, P2, | ¹H NMR (600 MHz, DMSO-d₆), characteristic peaks, |
| | C22^{22,23,24} | aliphatic integrations are approximate: δ 11.44 (br s, 1H), |
| | | 10.39 (s, 1H), 9.51 (br s, 1H), 8.44 (s, 1H), 8.35 (br t, J = |
| | | 6 Hz, 1H), 8.25 (br s, 1H), 8.18 (d, J = 9.5 Hz, 1H), 7.82 |
| | | (AB quartet, J_{AB} = 8.8 Hz, Δv_{AB} = 29.4 Hz, 2H), 7.52 (d, J = |
| | | 9.6 Hz, 1H), 7.32 (br s, 1H), 7.32-7.28 (m, 1H), 7.31 |
| | | (AB quartet, upfield doublet is broadened, J_{AB} = 7.8 Hz, |
| | | Δv_{AB} = 60.4 Hz, 2H), 4.64-4.40 (m, 4H), 3.84-3.74 (m, |
| | | 1H), 3.72-3.59 (m, 3H), 3.58-3.51 (m, 1H), 3.23-3.03 |
| | | (m, 7H), 2.80 (ddd, component of ABXY system, J = 16.1, |
| | | 9.7, 6.1 Hz, 1H), 2.69 (ddd, component of ABXY system, |
| | | J = 16.6, 5.5, 5.5 Hz, 1H), 2.22 (s, 3H), 2.21-2.13 (m, |
| | | 3H), 1.98-1.63 (m, 7H), 1.30-1.15 (m, 4H); 894.0 |
| 50 | Example | ¹H NMR (600 MHz, DMSO-d₆), characteristic peaks, |
| | 14²⁵, C121, | aliphatic integrations are approximate: δ 10.75 (s, 1H), |
| | C176 | 8.83 (s, 2H), 8.50 (br d, J = 7.0 Hz, 1H), 8.42 (s, 1H), 8.37- |
| | | 8.31 (m, 1H), 7.88 (s, 1H), 7.82-7.74 (m, 3H), 7.33 (br |
| | | d, J = 8.7 Hz, 1H), 7.32-7.27 (m, 1H), 7.18 (br d, J = 7 |
| | | Hz, 1H), 4.54-4.43 (m, 1H), 4.37-4.08 (br m, 2H), 3.85- |
| | | 3.79 (m, 2H), 3.21-3.15 (m, 2H), 2.89-2.81 (m, 2H), |
| | | 2.20-2.12 (m, 2H), 1.98-1.86 (m, 4H), 1.73-1.62 (m, |
| | | 1H), 1.28-1.17 (m, 2H); 808.4 |
| 51 | Example 14; | ¹H NMR (600 MHz, DMSO-d₆), characteristic peaks: δ |
| | C152, P245 | 11.44 (br s, 1H), 11.13 (s, 1H), 9.77 (br s, 1H), 8.35- |
| | | 8.30 (m, 1H), 8.22 (s, 1H), 7.55 (d, J = 9.2 Hz, 1H), 7.35 |
| | | (s, 1H), 7.31-7.27 (m, 1H), 7.23 (AB quartet, J_{AB} = 8.1 |
| | | Hz, Δv_{AB} = 20.2 Hz, 2H), 6.93-6.88 (m, 2H), 5.42 (dd, J = |
| | | 13.0, 5.4 Hz, 1H), 4.47-4.39 (m, 2H), 4.39-4.32 (m, |
| | | 1H), 3.90-3.69 (m, 2H), 3.16 (t, J = 6.3 Hz, 2H), 3.07- |
| | | 2.85 (m, 3H), 2.79-2.68 (m, 1H), 2.68-2.61 (m, 1H), |
| | | 2.14-2.07 (m, 2H), 2.07-2.00 (m, 1H), 1.95-1.79 (m, |
| | | 4H), 1.70-1.59 (m, 1H), 1.26-1.14 (m, 2H); 759.4 |
| 52 | Example 14; | ¹H NMR (600 MHz, DMSO-d₆), characteristic peaks: δ |
| | C152, P244 | 11.42 (br s, 1H), 11.13 (s, 1H), 9.57 (br s, 1H), 8.32 (br t, |
| | | J = 6 Hz, 1H), 8.22 (br s, 1H), 7.60-7.48 (m, 1H), 7.29 |
| | | (ddd, J = 11.0, 6.3, 2.2 Hz, 1H), 5.50-5.37 (m, 1H), 4.82- |
| | | 4.64 (m, 1H), 4.41-4.30 (m, 1H), 3.16 (t, J = 6.1 Hz, |
| | | 2H), 2.95-2.85 (m, 1H), 2.78-2.67 (m, 1H), 2.14-2.06 |
| | | (m, 2H), 2.05-1.98 (m, 1H), 1.94-1.79 (m, 4H), 1.69- |
| | | 1.59 (m, 1H), 1.25-1.14 (m, 2H); 759.4 |
| 53 | Example 17; | 2.21 minutes²⁶; 836.4 |
| | C176, P241 | |
| 54 | Example | ¹H NMR (600 MHz, DMSO-d₆), characteristic peaks, |
| | 17²⁷; C118, | integrations are approximate: δ 10.43 (s, 1H), 8.86 (s, |
| | C176 | 2H), 8.64 (br s, 1H), 8.42 (s, 1H), 8.35 (br t, J = 6 Hz, 1H), |
| | | 8.02 (s, 1H), 7.88 (br s, 1H), 7.78 (br d, J = 8.7 Hz, 1H), |
| | | 7.61 (d, J = 9.2 Hz, 1H), 7.34 (dd, J = 8.7, 1.5 Hz, 1H), |
| | | 7.29 (ddd, J = 11.0, 6.3, 2.2 Hz, 1H), 7.23 (dd, J = 9.2, |
| | | 1.5 Hz, 1H), 4.87-4.69 (m, 2H), 4.52-4.44 (m, 1H), |
| | | 3.76 (t, J = 6.7 Hz, 2H), 3.18 (t, J = 6.3 Hz, 2H), 3.11- |
| | | 3.05 (m, 2H), 2.77 (t, J = 6.7 Hz, 2H), 2.19-2.12 (m, |
| | | 2H), 1.96-1.86 (m, 4H), 1.71-1.62 (m, 1H), 1.27-1.18 |
| | | (m, 2H); 822.5 |
| 55 | Example 14; | ¹H NMR (600 MHz, DMSO-d₆), characteristic peaks: δ |
| | C170, P240 | 11.10 (s, 1H), 8.21 (br t, J = 6 Hz, 1H), 7.98 (d, J = 9.5 |
| | | Hz, 1H), 7.52 (d, J = 9.6 Hz, 1H), 7.28 (ddd, J = 11.0, 6.2, |
| | | 2.2 Hz, 1H), 7.14 (br s, 1H), 7.04 (AB quartet, upfield |
| | | doublet is broadened, J_{AB} = 8.0 Hz, Δv_{AB} = 70.9 Hz, 2H), |
| | | 5.36 (dd, J = 13.0, 5.4 Hz, 1H), 4.81-4.44 (m, 2H), 3.34 |
| | | (s, 3H), 3.11-3.04 (m, 4H), 2.95-2.84 (m, 1H), 2.75- |
| | | 2.66 (m, 1H), 2.66-2.59 (m, 1H), 2.04-1.93 (m, 7H), |
| | | 1.59-1.51 (m, 6H); 829.4 |
| 56 | Example | ¹H NMR (600 MHz, DMSO-d₆), characteristic peaks: δ |
| | 14²⁸; C128, | 11.50 (v br s, 1H), 11.11 (s, 1H), 9.84 (br s, 1H), 8.34 (br |
| | C152 | t, J = 6 Hz, 1H), 8.24 (s, 1H), 7.58 (d, J = 8.9 Hz, 1H), |
| | | 7.29 (ddd, J = 11.1, 6.2, 2.3 Hz, 1H), 7.10-7.03 (m, 2H), |
| | | 6.99-6.94 (m, 3H), 5.38 (dd, J = 12.8, 5.4 Hz, 1H), 4.37 |
| | | (tt, J = 11.9, 3.9 Hz, 1H), 3.94-3.81 (m, 2H), 3.81-3.69 |
| | | (m, 2H), 3.17 (t, J = 6.4 Hz, 2H), 3.08-2.95 (m, 2H), |
| | | 2.94-2.84 (m, 1H), 2.75-2.66 (m, 1H), 2.66-2.59 (m, |
| | | 1H), 2.15-2.07 (m, 2H), 2.03-1.96 (m, 1H), 1.94-1.81 |
| | | (m, 4H), 1.70-1.60 (m, 1H), 1.25-1.15 (m, 2H); 773.5 |
| 57 | Example | 3.06 minutes³⁰; 894.7 |
| | 20²⁹; C144, | |
| | P239 | |
| 58 | Example 57; | 2.66 minutes²⁶; 850.6 |
| | C144, P239 | |
| 59 | Example 57; | 2.35 minutes²⁶; 935.7 |
| | C144, P239, | |
| | C125 | |
| 60 | Example 14; | ¹H NMR (600 MHz, DMSO-d₆), characteristic peaks: δ |
| | C61, P240 | 11.39 (br s, 1H), 11.10 (s, 1H), 9.96 (br s, 1H), 8.76 (d, J = |
| | | 4.9 Hz, 1H), 8.19 (br t, J = 6 Hz, 1H), 7.43 (d, J = 4.9 |
| | | Hz, 1H), 7.27 (ddd, J = 11.0, 6.2, 2.2 Hz, 1H), 7.14 (br s, |
| | | 1H), 7.03 (AB quartet, upfield doublet is broadened, J_{AB} = |
| | | 8.1 Hz, Δv_{AB} = 72.3 Hz, 2H), 5.36 (dd, J = 13.0, 5.4 Hz, |
| | | 1H), 4.90-4.62 (m, 2H), 3.83-3.58 (m, 2H), 3.25-3.11 |
| | | (m, 2H), 3.11-3.02 (m, 4H), 2.95-2.86 (m, 1H), 2.76- |
| | | 2.66 (m, 1H), 2.66-2.59 (m, 1H), 2.04-1.96 (m, 1H), |
| | | 1.94-1.85 (m, 6H), 1.59-1.49 (m, 6H); 829.5 |
| 61 | Example 14; | ¹H NMR (600 MHz, DMSO-d₆), characteristic peaks: δ |
| | C61, P241 | 11.39 (br s, 1H), 10.43 (s, 1H), 9.77 (br s, 1H), 8.75 (d, J = |
| | | 4.9 Hz, 1H), 8.56 (s, 1H), 8.19 (br t, J = 6 Hz, 1H), 7.99 |
| | | (s, 1H), 7.58 (d, J = 9.1 Hz, 1H), 7.42 (d, J = 4.9 Hz, 1H), |
| | | 7.27 (ddd, J = 10.9, 6.2, 2.2 Hz, 1H), 7.20 (dd, J = 9.1, |
| | | 1.4 Hz, 1H), 4.84-4.67 (m, 2H), 3.76 (t, J = 6.7 Hz, 2H), |
| | | 3.70-3.58 (m, 2H), 3.19-3.06 (m, 4H), 3.07 (d, J = 6.3 |
| | | Hz, 2H), 2.77 (t, J = 6.7 Hz, 2H), 2.73-2.68 (m, 2H), |
| | | 2.10-2.02 (m, 2H), 1.92-1.86 (m, 6H), 1.57-1.50 (m, |
| | | 6H); 814.5 |
| 62 | P239, | 2.59 minutes²⁶, 866.5 |
| | C144³¹ | |
| 63 | Example 62; | 2.41 minutes²⁶; 850.6 |
| | P239, C144 | |
| 64 | Example | ¹H NMR (600 MHz, DMSO-d₆), characteristic peaks, |
| | 13³²; P229, | aliphatic integrations are approximate: δ 11.09 (s, 1H), |
| | P240 | 8.37-8.32 (m, 2H), 7.64 (d, J = 8.5 Hz, 1H), 7.44 (s, 1H), |
| | | 7.28 (ddd, J = 11.0, 6.2, 2.2 Hz, 1H), 7.09 (br s, 1H), 7.00 |
| | | (AB quartet, upfield doublet is broadened, J_{AB} = 8.1 Hz, |
| | | Δv_{AB} = 76.8 Hz, 2H), 6.91 (br d, J = 8.5 Hz, 1H), 5.33 (dd, |
| | | J = 12.9, 5.4 Hz, 1H), 4.43 (tt, J = 11.7, 3.8 Hz, 1H), 3.32 |
| | | (s, 3H), 3.19-3.11 (m, 3H), 3.07-2.94 (m, 2H), 2.93- |
| | | 2.84 (m, 1H), 2.73-2.65 (m, 1H), 2.65-2.59 (m, 1H), |
| | | 2.16-2.09 (m, 2H), 2.03-1.96 (m, 1H), 1.94-1.83 (m, |
| | | 4H), 1.83-1.75 (m, 1H), 1.70-1.59 (m, 2H), 1.26-1.16 |
| | | (m, 2H); 822.9 |
| 65 | Example | ¹H NMR (600 MHz, DMSO-d₆), characteristic peaks, |
| | 35³³; P229, | aliphatic integrations are approximate: δ 11.41 (br s, 1H), |
| | P231 | 11.08 (s, 1H), 8.37 (s, 1H), 8.33 (br t, J = 6 Hz, 1H), 7.69- |
| | | 7.65 (m, 2H), 7.52 (br d, J = 9.0 Hz, 1H), 7.29 (ddd, J = |
| | | 11.0, 6.2, 2.2 Hz, 1H), 7.06 (br s, 1H), 6.96 (AB quartet, |
| | | J_{AB} = 8.1 Hz, Δv_{AB} = 70.7 Hz, 2H), 5.33 (dd, J = 13.0, 5.4 |
| | | Hz, 1H), 4.44 (tt, J = 11.7, 3.8 Hz, 1H), 3.98-3.91 (m, |
| | | 1H), 3.88-3.81 (m, 1H), 3.32 (s, 3H), 3.17 (t, J = 6.3 Hz, |
| | | 2H), 2.93-2.85 (m, 1H), 2.74-2.58 (m, 5H), 2.42-2.35 |
| | | (m, 1H), 2.29-2.07 (m, 5H), 2.03-1.96 (m, 1H), 1.95- |
| | | 1.81 (m, 5H), 1.71-1.61 (m, 1H), 1.27-1.17 (m, 2H); 891.6 |
| 66 | Example | ¹H NMR (600 MHz, DMSO-d₆), characteristic peaks, |
| | 14³⁴; P10, | aliphatic integrations are approximate: δ 11.09 (s, 1H), |
| | P231 | 8.18 (br t, J = 6 Hz, 1H), 8.07 (d, J = 9.5 Hz, 1H), 7.50 (d, |
| | | J = 9.7 Hz, 1H), 7.27 (ddd, J = 11.0, 6.2, 2.3 Hz, 1H), |
| | | 7.09 (br s, 1H), 6.98 (AB quartet, upfield doublet is |
| | | broadened, J_{AB} = 8.0 Hz, Δv_{AB} = 71.6 Hz, 2H), 5.35 (dd, J = |
| | | 13.0, 5.5 Hz, 1H), 4.90-4.25 (v br m, 2H), 3.34 (s, 3H, |
| | | assumed; largely obscured by water peak), 3.12-3.05 |
| | | (m, 4H), 2.94-2.86 (m, 1H), 2.74-2.66 (m, 3H), 2.65- |
| | | 2.60 (m, 1H), 2.04-1.96 (m, 3H), 1.94-1.86 (m, 6H), |
| | | 1.58-1.50 (m, 6H); 843.5 |
| 67 | Example | ¹H NMR (600 MHz, DMSO-d₆), characteristic peaks: δ |
| | 66³⁵; P10, | 11.38 (br s, 1H), 11.09 (s, 1H), 9.81 (br s, 1H), 9.01 (s, |
| | P231 | 2H), 8.18 (br t, J = 6 Hz, 1H), 7.27 (ddd, J = 11.0, 6.2, 2.3 |
| | | Hz, 1H), 7.08 (br s, 1H), 6.98 (AB quartet, upfield doublet |
| | | is broadened, J_{AB} = 8.1 Hz, Δv_{AB} = 75.1 Hz, 2H), 5.35 (dd, |
| | | J = 12.9, 5.4 Hz, 1H), 4.97-4.67 (m, 2H), 3.72-3.51 |
| | | (m, 2H), 3.34 (s, 3H), 3.19-3.06 (m, 2H), 3.07 (d, J = 6.3 |
| | | Hz, 2H), 2.95-2.85 (m, 1H), 2.75-2.65 (m, 3H), 2.65- |
| | | 2.59 (m, 1H), 2.06-1.96 (m, 3H), 1.91-1.83 (m, 6H), |
| | | 1.57-1.48 (m, 6H); 843.4 |
| 68 | Example 42; | ¹H NMR (600 MHz, DMSO-d₆), characteristic peaks, |
| | C144, P247, | aliphatic integrations are approximate: δ 11.41 (br s, 1H), |
| | P239 | 10.38 (s, 1H), 10.05 (br s, 1H), 8.85 (s, 2H), 8.42 (s, 1H), |
| | | 8.34 (br t, J = 6 Hz, 1H), 7.89 (s, 1H), 7.78 (d, J = 8.7 Hz, |
| | | 1H), 7.37-7.32 (m, 3H), 7.32-7.26 (m, 2H), 4.94-4.67 |
| | | (m, 1H), 4.53-4.44 (m, 1H), 4.18-3.98 (m, 2H), 3.98- |
| | | 3.84 (m, 1H), 3.84-3.75 (m, 1H), 3.58-3.49 (m, 1H), |
| | | 3.18 (t, J = 6.3 Hz, 2H), 2.79 (ddd, component of ABXY |
| | | system, J = 16.0, 9.8, 6.0 Hz, 1H), 2.69 (ddd, component |
| | | of ABXY system, J = 16.9, 5.5, 5.5 Hz, 1H), 2.32-2.19 |
| | | (m, 1H), 2.22 (s, 3H), 2.19-2.13 (m, 2H), 2.05-1.85 (m, |
| | | 5H), 1.82-1.51 (m, 5H), 1.28-1.19 (m, 2H); 935.7 |
| 69 | Example 66; | ¹H NMR (600 MHz, DMSO-d₆), characteristic peaks: δ |
| | P10, P231 | 11.38 (br s, 1H), 11.09 (s, 1H), 9.80 (br s, 1H), 8.83 (s, |
| | | 1H), 8.57 (s, 1H), 8.18 (br t, J = 6 Hz, 1H), 7.30-7.24 |
| | | (m, 1H), 7.08 (s, 1H), 6.99 (AB quartet, J_{AB} = 8.0 Hz, Δv_{AB} = |
| | | 74.5 Hz, 2H), 5.35 (dd, J = 13.0, 5.5 Hz, 1H), 4.74- |
| | | 4.56 (m, 2H), 3.75-3.55 (m, 2H), 3.20-3.08 (m, 3H), |
| | | 3.07 (d, J = 6.3 Hz, 2H), 2.94-2.86 (m, 1H), 2.75-2.65 |
| | | (m, 3H), 2.65-2.59 (m, 1H), 2.07-1.96 (m, 3H), 1.92- |
| | | 1.85 (m, 6H), 1.57-1.50 (m, 6H); 844.1 |
| 70 | Example 13; | ¹H NMR (600 MHz, DMSO-d₆), characteristic peaks: δ |
| | C149, P248 | 11.41 (br s, 1H), 10.48 (s, 1H), 10.03 (br s, 1H), 8.83 (s, |
| | | 2H), 8.51 (br s, 1H), 8.41 (s, 1H), 8.34 (br t, J = 6 Hz, 1H), |
| | | 8.21 (s, 1H), 8.15 (br s, 1H), 7.76 (d, half of AB quartet, J = |
| | | 8.8 Hz, 1H), 7.74-7.68 (m, 2H), 7.34 (d, J = 9.2 Hz, |
| | | 1H), 7.30 (ddd, J = 11.1, 6.3, 2.3 Hz, 1H), 4.66-4.28 (m, |
| | | 4H), 3.79 (t, J = 6.7 Hz, 2H), 3.18 (t, J = 6.3 Hz, 2H), 2.79 |
| | | (t, J = 6.7 Hz, 2H), 2.21-2.13 (m, 2H), 1.98-1.86 (m, |
| | | 4H), 1.73-1.62 (m, 1H), 1.30-1.18 (m, 2H); 808.5 |
| 71 | Example | ¹H NMR (600 MHz, DMSO-d₆), characteristic peaks, |
| | 20³⁶; C149, | aliphatic integrations are approximate: δ 11.41 (br s, 1H), |
| | P239 | 10.38 (s, 1H), 10.07 (br s, 1H), 8.83 (br s, 1H), 8.53 (s, |
| | | 1H), 8.41 (s, 1H), 8.34 (br t, J = 6 Hz, 1H), 8.21 (br s, 1H), |
| | | 7.74 (AB quartet, upfield doublet is broadened, J_{AB} = 8.8 |
| | | Hz, Δv_{AB} = 29.3 Hz, 2H), 7.37-7.32 (m, 2H), 7.32-7.28 |
| | | (m, 1H), 7.29-7.26 (m, 1H), 4.67-4.43 (m, 2H), 4.17- |
| | | 3.99 (m, 2H), 3.98-3.86 (m, 1H), 3.84-3.75 (m, 1H), |
| | | 3.18 (t, J = 6.3 Hz, 2H), 2.79 (ddd, component of ABXY |
| | | system, J = 16.4, 9.8, 6.0 Hz, 1H), 2.69 (ddd, component |
| | | of ABXY system, J = 16.6, 5.5, 5.3 Hz, 1H), 2.32-2.23 |
| | | (m, 1H), 2.22 (s, 3H), 2.20-2.14 (m, 2H), 1.97-1.86 (m, |
| | | 4H), 1.84-1.50 (m, 5H), 1.29-1.18 (m, 2H); 935.7 |
| 72 | Example 71; | ¹H NMR (600 MHz, DMSO-d₆), characteristic peaks, |
| | C149, P239 | aliphatic integrations are approximate: δ 11.42 (br s, 1H), |
| | | 10.38 (s, 1H), 9.88 (br s, 1H), 8.84 (s, 1H), 8.54 (s, 1H), |
| | | 8.41 (s, 1H), 8.34 (br t, J = 6 Hz, 1H), 8.22 (br s, 1H), |
| | | 7.74 (AB quartet, upfield doublet is broadened, J_{AB} = 8.8 |
| | | Hz, Δv_{AB} = 30.3 Hz, 2H), 7.31 (s, 1H), 7.31-7.27 (m, |
| | | 1H), 7.30 (AB quartet, upfield doublet is broadened, J_{AB} = |
| | | 7.9 Hz, Δv_{AB} = 60.0 Hz, 2H), 4.61-4.43 (m, 3H), 3.87- |
| | | 3.73 (m, 3H), 3.18 (t, J = 6.3 Hz, 2H), 3.03-2.90 (m, |
| | | 2H), 2.79 (ddd, component of ABXY system, J = 16.3, |
| | | 9.6, 6.3 Hz, 1H), 2.69 (ddd, component of ABXY system, |
| | | J = 16.6, 5.5, 5.5 Hz, 1H), 2.29-2.19 (m, 2H), 2.22 (s, |
| | | 3H), 2.19-2.14 (m, 2H), 2.10-1.99 (m, 2H), 1.98-1.84 |
| | | (m, 4H), 1.75-1.47 (m, 6H), 1.29-1.18 (m, 2H); 919.9 |
| 73 | Example | ¹H NMR (600 MHz, DMSO-d₆), characteristic peaks, |
| | 11^{37,38}; | aliphatic integrations are approximate: δ 11.46 (br s, 1H), |
| | C126, C144 | 10.37 (s, 1H), 10.11 (br s, 1H), 8.85 (s, 2H), 8.42 (s, 1H), |
| | | 8.34 (br t, J = 6 Hz, 1H), 7.89 (br s, 1H), 7.78 (d, J = 8.7 |
| | | Hz, 1H), 7.37-7.32 (m, 3H), 7.32-7.26 (m, 2H), 4.98- |
| | | 4.59 (m, 2H), 4.53-4.43 (m, 1H), 4.17-3.97 (m, 4H), |
| | | 3.97-3.84 (m, 1H), 3.83-3.74 (m, 1H), 3.56-3.50 (m, |
| | | 1H, assumed; partially obscured by water peak), 3.18 (t, J = |
| | | 6.3 Hz, 2H), 2.79 (ddd, component of ABXY system, J = |
| | | 16.6, 9.5, 6.1 Hz, 1H), 2.69 (ddd, component of ABXY |
| | | system, J = 16.6, 5.4, 5.4 Hz, 1H), 2.32-2.24 (m, 1H), |
| | | 2.21 (s, 3H), 2.19-2.12 (m, 2H), 2.04-1.85 (m, 5H), |
| | | 1.83-1.50 (m, 5H), 1.27-1.18 (m, 2H); 935.6 |
| 74 | Example | ¹H NMR (600 MHz, DMSO-d₆), characteristic peaks: δ |
| | 8³⁹; P229, | 11.43 (br s, 1H), 11.10 (s, 1H), 9.64 (br s, 1H), 8.33 (br t, |
| | P240 | J = 6 Hz, 1H), 8.20 (s, 1H), 7.52 (d, J = 9.1 Hz, 1H), 7.29 |
| | | (ddd, J = 11.0, 6.3, 2.3 Hz, 1H), 7.13-7.07 (m, 2H), 6.99- |
| | | 6.93 (m, 2H), 6.88 (br s, 1H), 5.36 (dd, J = 12.9, 5.4 Hz, |
| | | 1H), 4.36 (tt, J = 11.5, 3.8 Hz, 1H), 3.98-3.86 (m, 2H), |
| | | 3.34 (s, 3H), 3.16 (t, J = 6.3 Hz, 2H), 3.13-3.01 (m, 4H), |
| | | 3.01-2.85 (m, 3H), 2.75-2.66 (m, 1H), 2.66-2.59 (m, |
| | | 1H), 2.45-2.36 (m, 1H), 2.15-2.08 (m, 2H), 2.04-1.96 |
| | | (m, 1H), 1.95-1.74 (m, 6H), 1.70-1.59 (m, 2H), 1.26- |
| | | 1.15 (m, 2H); 844.1 |
| 75 | Example | ¹H NMR (600 MHz, DMSO-d₆), characteristic peaks: δ |
| | 8³⁹; P229, | 11.43 (br s, 1H), 11.09 (s, 1H), 9.44 (br s, 1H), 8.33 (br t, |
| | P231 | J = 6 Hz, 1H), 8.19 (s, 1H), 7.51 (d, J = 9.1 Hz, 1H), 7.29 |
| | | (ddd, J = 11.0, 6.2, 2.3 Hz, 1H), 7.07 (d, J = 1.5 Hz, 1H), |
| | | 7.05 (d, J = 8.0 Hz, 1H), 6.95-6.90 (m, 2H), 6.86 (br s, |
| | | 1H), 5.35 (dd, J = 12.9, 5.5 Hz, 1H), 4.35 (tt, J = 11.6, 3.8 |
| | | Hz, 1H), 3.93-3.79 (m, 2H), 3.33 (s, 3H), 3.16 (t, J = 6.3 |
| | | Hz, 2H), 3.13-2.82 (m, 7H), 2.75-2.59 (m, 4H), 2.41- |
| | | 2.32 (m, 1H), 2.14-2.07 (m, 2H), 2.07-1.95 (m, 3H), |
| | | 1.94-1.80 (m, 4H), 1.80-1.69 (m, 2H), 1.69-1.55 (m, |
| | | 2H), 1.25-1.15 (m, 2H); 858.1 |
| 76 | Example | ¹H NMR (600 MHz, DMSO-d₆) δ 11.42 (br s, 1H), 11.09 |
| | 74⁴⁰; P230, | (s, 1H), 9.64 (br s, 1H), 8.69 (s, 2H), 8.53-8.51 (m, 1H), |
| | P241 | 8.43 (br s, 1H), 8.35 (br t, J = 6 Hz, 1H), 8.01 (dd, J = 8.8, |
| | | 1.4 Hz, 1H), 7.75 (br d, J = 8.9 Hz, 1H), 7.30 (ddd, J = |
| | | 11.0, 6.2, 2.3 Hz, 1H), 7.10 (d, J = 1.5 Hz, 1H), 6.99 (AB |
| | | quartet, upfield doublet is broadened, J_{AB} = 8.0 Hz, Δv_{AB} = |
| | | 72.1 Hz, 2H), 5.35 (dd, J = 13.0, 5.5 Hz, 1H), 4.49 (tt, J = |
| | | 11.5, 2.9 Hz, 1H), 4.14-4.01 (m, 2H), 3.70-3.58 (m, |
| | | 2H), 3.34 (s, 3H, assumed; largely obscured by water |
| | | peak), 3.25-3.11 (m, 8H), 2.95-2.85 (m, 1H), 2.75- |
| | | 2.66 (m, 3H), 2.66-2.59 (m, 1H), 2.21-2.14 (m, 2H), |
| | | 2.08-1.97 (m, 3H), 1.97-1.87 (m, 4H), 1.74-1.64 (m, |
| | | 1H), 1.29-1.18 (m, 2H); 866.1 |
| 77 | Example | ¹H NMR (600 MHz, DMSO-d₆), characteristic peaks, |
| | 8³⁹; P229 | integrations are approximate: δ 11.42 br (s, 1H), 11.13 (s, |
| | P245 | 1H), 9.44 (br s, 1H), 8.36-8.30 (m, 1H), 8.18 (s, 1H), |
| | | 7.49 (d, J = 9.1 Hz, 1H), 7.22 (d, J = 8.4 Hz, 1H), 6.91 (br |
| | | d, J = 9.1 Hz, 1H), 6.87 (d, J = 8.6 Hz, 1H), 5.44-5.38 |
| | | (m, 1H), 4.40 (s, 2H), 3.18-3.14 (m, 2H), 2.76-2.67 (m, |
| | | 1H), 2.14-2.06 (m, 2H), 2.05-1.99 (m, 1H), 1.94-1.79 |
| | | (m, 4H), 1.72-1.57 (m, 3H), 1.25-1.14 (m, 2H); 830.1 |
| 78 | Example 15; | ¹H NMR (600 MHz, DMSO-d₆), characteristic peaks: δ |
| | C161, P245 | 11.08 (s, 1H), 8.72 (s, 2H), 8.40 (s, 1H), 8.34 (br t, J = 6 |
| | | Hz, 1H), 7.82 (s, 1H), 7.75 (d, J = 8.6 Hz, 1H), 7.33- |
| | | 7.26 (m, 2H), 7.20-6.99 (m, 3H), 5.38-5.30 (m, 1H), |
| | | 4.52-4.42 (m, 1H), 4.04-3.87 (m, 4H), 3.75-3.65 (m, |
| | | 2H), 3.17 (t, J = 6.3 Hz, 2H), 2.92-2.82 (m, 1H), 2.73- |
| | | 2.63 (m, 1H), 2.63-2.57 (m, 1H), 2.20-2.11 (m, 2H), |
| | | 2.03-1.96 (m, 1H), 1.96-1.86 (m, 4H), 1.71-1.62 (m, |
| | | 1H), 1.28-1.17 (m, 2H); 879.6 |
| 79 | Example 13; | ¹H NMR (600 MHz, DMSO-d₆), characteristic peaks, |
| | C161, P240 | integrations are approximate: δ 11.45 (br s, 1H), 11.10 (s, |
| | | 1H), 9.93 (br s, 1H), 8.80 (s, 2H), 8.42 (s, 1H), 8.37- |
| | | 8.32 (m, 1H), 7.86 (s, 1H), 7.78 (d, J = 8.6 Hz, 1H), 7.34- |
| | | 7.26 (m, 2H), 7.13-7.07 (m, 2H), 6.96 (d, J = 8.1 Hz, |
| | | 1H), 5.35 (dd, J = 12.9, 5.9 Hz, 1H), 4.53-4.43 (m, 1H), |
| | | 4.39-3.79 (m, 7H), 3.34 (s, 3H), 3.18 (t, J = 6.4 Hz, 2H), |
| | | 2.95-2.84 (m, 1H), 2.74-2.59 (m, 2H), 2.19-2.12 (m, |
| | | 2H), 2.04-1.97 (m, 1H), 1.97-1.86 (m, 4H), 1.42-1.36 |
| | | (m, 1H); 893.6 |
| 80 | C66, C144⁴¹ | ¹H NMR (600 MHz, DMSO-d₆) δ 11.47 (br s, 1H), 11.09 |
| | | (s, 1H), 8.73 (s, 2H), 8.40 (s, 1H), 8.35 (br t, J = 6 Hz, |
| | | 1H), 7.82 (br s, 1H), 7.75 (d, J = 8.7 Hz, 1H), 7.33-7.27 |
| | | (m, 2H), 7.10 (s, 1H), 6.97 (AB quartet, J_{AB} = 8.0 Hz, Δv_{AB} = |
| | | 53.8 Hz, 2H), 5.33 (dd, J = 13.0, 5.4 Hz, 1H), 4.52- |
| | | 4.42 (m, 1H), 3.90-3.81 (m, 2H), 3.77-3.70 (m, 2H), |
| | | 3.63-3.55 (m, 1H), 3.35 (s, 3H), 3.18 (t, J = 6.3 Hz, 2H), |
| | | 2.95-2.85 (m, 1H), 2.74-2.59 (m, 2H), 2.35-2.29 (m, |
| | | 2H), 2.20-2.12 (m, 2H), 2.04-1.97 (m, 1H), 1.97-1.86 |
| | | (m, 6H), 1.79-1.73 (m, 2H), 1.71-1.62 (m, 1H), 1.62- |
| | | 1.57 (m, 2H), 1.27-1.18 (m, 2H); 862.7 |
| 81 | Example | ¹H NMR (600 MHz, DMSO-d₆), characteristic peaks, |
| | 13⁴²; P10, | aliphatic integrations are approximate: δ 11.09 (s, 1H), |
| | P231 | 8.13 (br t, J = 6 Hz, 1H), 7.51 (br s, 1H), 7.46 (br d, half of |
| | | AB quartet, J = 7.6 Hz, 1H), 7.43 (dd, component of ABX |
| | | system, J = 8.0, 7.6 Hz, 1H), 7.29-7.22 (m, 2H), 7.07 (d, |
| | | J = 1.5 Hz, 1H), 7.01 (d, half of AB quartet, J = 8.0 Hz, |
| | | 1H), 6.90 (dd, component of ABX system, J = 8.0, 1.6 Hz, |
| | | 1H), 5.33 (dd, J = 12.9, 5.4 Hz, 1H), 3.33 (s, 3H), 3.31- |
| | | 3.25 (m, 4H), 3.07 (d, J = 6.2 Hz, 2H), 2.89 (ddd, J = |
| | | 17.0, 13.5, 5.4 Hz, 1H), 2.74-2.59 (m, 7H), 2.04-1.97 |
| | | (m, 1H), 1.92-1.80 (m, 8H), 1.56-1.48 (m, 6H); 841.5 |
| 82 | Example 71; | characteristic peaks, integrations are approximate: δ |
| | C152, P239 | 10.36 (s, 1H), 8.17 (br s, 1H), 8.01-7.90 (m, 1H), 7.48 |
| | | (d, J = 9.1 Hz, 1H), 7.31 (s, 1H), 7.29 (AB quartet, J_{AB} = |
| | | 7.8 Hz, Δv_{AB} = 39.7 Hz, 2H), 7.21-7.13 (m, 1H), 6.89 |
| | | (dd, J = 9.1, 1.9 Hz, 1H), 6.77 (s, 1H), 4.39-4.28 (m, |
| | | 1H), 3.86-3.74 (m, 1H), 3.58-3.47 (m, 1H), 3.15 (t, J = |
| | | 6.1 Hz, 2H), 3.13-3.05 (m, 4H), 2.84-2.64 (m, 3H), |
| | | 2.46-2.38 (m, 4H), 2.21 (s, 3H), 2.15-2.06 (m, 2H), |
| | | 2.06-1.96 (m, 2H), 1.94-1.78 (m, 4H), 1.70-1.41 (m, |
| | | 7H), 1.27-1.13 (m, 2H); 841.3 |
| 83 | Example 12; | characteristic peaks: δ 11.11 (s, 1H), 8.75 (s, 2H), 8.40 |
| | P16, P245 | (s, 1H), 8.19 (br s, 1H), 7.85 (s, 1H), 7.74 (d, J = 8.7 Hz, |
| | | 1H), 7.70-7.62 (m, 1H), 7.30 (d, J = 8.6 Hz, 1H), 7.16 (s, |
| | | 1H), 7.14-7.07 (m, 1H), 7.05 (AB quartet, J_{AB} = 7.9 Hz, |
| | | Δv_{AB} = 20.7 Hz, 2H), 5.37 (dd, J = 12.8, 5.4 Hz, 1H), 3.84- |
| | | 3.75 (m, 4H), 3.55 (s, 2H), 3.35 (s, 3H, assumed; |
| | | partially obscured by water peak), 3.11 (d, J = 6.1 Hz, |
| | | 2H), 2.97-2.84 (m, 1H), 2.79-2.57 (m, 2H), 2.22-2.11 |
| | | (m, 6H), 2.07-1.97 (m, 1H), 1.72-1.59 (m, 6H); 863.3 |
| 84 | Example 12; | characteristic peaks, aliphatic integrations are |
| | P232, P245 | approximate: δ 11.10 (s, 1H), 8.84 (s, 2H), 8.49 (d, J = |
| | | 7.1 Hz, 1H), 8.12 (br t, J = 6 Hz, 1H), 7.81 (br s, 1H), 7.61 |
| | | (s, 1H), 7.27 (ddd, J = 11.0, 6.3, 2.3 Hz, 1H), 7.24-7.17 |
| | | (m, 2H), 7.07 (AB quartet, J_{AB} = 8.0 Hz, Δv_{AB} = 19.7 Hz, |
| | | 2H), 5.37 (dd, J = 12.8, 5.4 Hz, 1H), 3.94-3.75 (m, 4H), |
| | | 3.75-3.58 (m, 2H), 3.07 (d, J = 6.2 Hz, 2H), 2.98-2.85 |
| | | (m, 1H), 2.80-2.5 (m, 6H, assumed; partially obscured |
| | | by solvent peak), 2.08-1.96 (m, 2H), 1.88-1.79 (m, |
| | | 6H), 1.58-1.47 (m, 6H); 863.3 |
| 85 | Example 22; | characteristic peaks, aliphatic integrations are |
| | P229, P231 | approximate: δ 11.09 (s, 1H), 8.14 (s, 1H), 8.10-8.02 |
| | | (m, 1H), 7.47 (d, J = 9.1 Hz, 1H), 7.19 (ddd, J = 11.7, 6.9, |
| | | 2.2 Hz, 1H), 7.02 (br s, 1H), 6.92 (AB quartet, J_{AB} = 8.1 |
| | | Hz, Δv_{AB} = 52.3 Hz, 2H), 6.75 (br d, J = 9.0 Hz, 1H), 6.50 |
| | | (s, 1H), 5.33 (dd, J = 12.7, 5.4 Hz, 1H), 4.37-4.25 (m, |
| | | 1H), 3.41-3.33 (m, 2H), 3.29 (s, 3H), 3.19-3.09 (m, |
| | | 4H), 2.97-2.76 (m, 5H), 2.76-2.56 (m, 6H), 2.15-2.05 |
| | | (m, 2H), 2.03-1.94 (m, 1H), 1.93-1.73 (m, 5H), 1.69- |
| | | 1.56 (m, 1H), 1.27-1.10 (m, 2H); 813.3 |
| 86 | Example 32; | characteristic peaks, aliphatic integrations are |
| | P230, P231 | approximate: δ 11.10 (s, 1H), 8.75 (s, 1H), 8.64 (s, 1H), |
| | | 8.47 (s, 1H), 8.12-8.00 (m, 2H), 7.78 (d, J = 8.9 Hz, 1H), |
| | | 7.24-7.16 (m, 1H), 7.10 (s, 1H), 6.98 (AB quartet, J_{AB} = |
| | | 8.0 Hz, Δv_{AB} = 38.9 Hz, 2H), 5.35 (dd, J = 12.8, 5.4 Hz, |
| | | 1H), 4.58-4.44 (m, 1H), 3.98 (s, 4H), 3.34 (s, 3H, |
| | | assumed; partially obscured by water peak), 3.17 (t, J = |
| | | 6.4 Hz, 2H), 2.97-2.84 (m, 1H), 2.79-2.57 (m, 6H), |
| | | 2.23-2.14 (m, 2H), 2.05-1.81 (m, 7H), 1.75-1.61 (m, |
| | | 1H), 1.31-1.15 (m, 2H); 822.3 |
| 87 | Example 32; | characteristic peaks, aliphatic integrations are |
| | P230, P231 | approximate: δ 11.09 (s, 1H), 8.62 (br s, 1H), 8.61 (s, |
| | | 1H), 8.45 (s, 1H), 8.06 (dd, J = 8.8, 1.4 Hz, 1H), 7.98- |
| | | 7.88 (m, 1H), 7.76 (d, J = 8.9 Hz, 1H), 7.20-7.12 (m, |
| | | 1H), 7.08 (s, 1H), 6.96 (AB quartet, upfield doublet is |
| | | broadened, J_{AB} = 8.0 Hz, Δv_{AB} = 40.5 Hz, 2H), 5.34 (dd, J = |
| | | 12.6, 5.3 Hz, 1H), 4.57-4.45 (m, 1H), 3.63 (s, 2H), |
| | | 3.33 (s, 3H, assumed; partially obscured by water peak), |
| | | 3.17 (t, J = 6.3 Hz, 2H), 3.01-2.95 (m, 2H), 2.95-2.84 |
| | | (m, 1H), 2.84-2.79 (m, 2H), 2.77-2.57 (m, 5H), 2.23- |
| | | 2.13 (m, 2H), 2.05-1.82 (m, 7H), 1.74-1.61 (m, 1H), |
| | | 1.30-1.14 (m, 2H); 836.4 |
| 88 | Example 21; | characteristic peaks, aliphatic integrations are |
| | P230, P231 | approximate: δ 11.10 (s, 1H), 8.42 (s, 1H), 8.39 (s, 1H), |
| | | 8.30-8.22 (m, 2H), 8.14 (s, 1H), 7.82-7.71 (m, 3H), |
| | | 7.27 (ddd, J = 11.3, 6.5, 2.3 Hz, 1H), 7.09 (br s, 1H), 6.96 |
| | | (AB quartet, upfield doublet is broadened, J_{AB} = 8.1 Hz, |
| | | Δv_{AB} = 42.5 Hz, 2H), 5.35 (dd, J = 12.8, 5.5 Hz, 1H), 4.55- |
| | | 4.42 (m, 1H), 3.67 (s, 2H), 3.33 (s, 3H, assumed; |
| | | partially obscured by water peak), 3.17 (t, J = 6.4 Hz, 2H), |
| | | 2.97-2.84 (m, 3H), 2.79-2.61 (m, 5H), 2.23-2.13 (m, |
| | | 2H), 2.05-1.83 (m, 7H), 1.74-1.61 (m, 1H), 1.31-1.15 |
| | | (m, 2H); 835.3 |
| 89 | Example | characteristic peaks, aliphatic integrations are |
| | 20⁴³; C176, | approximate: δ 10.53 (s, 1H), 8.75 (s, 2H), 8.40 (s, 1H), |
| | P249 | 8.32 (br s, 2H), 7.85 (s, 1H), 7.75 (d, J = 8.6 Hz, 1H), 7.64 |
| | | (d, J = 8.1 Hz, 1H), 7.60-7.49 (m, 2H), 7.39 (d, J = 8.2 |
| | | Hz, 1H), 7.31 (d, J = 8.7 Hz, 1H), 7.07-6.97 (m, 1H), |
| | | 4.55-4.37 (m, 2H), 3.69-3.51 (m, 2H), 3.23-2.98 (m, |
| | | 3H), 2.88-2.65 (m, 3H), 2.46-2.37 (m, 4H), 2.26-2.08 |
| | | (m, 4H), 2.03-1.77 (m, 6H), 1.77-1.57 (m, 2H), 1.34- |
| | | 1.02 (m, 4H); 913.3 |
| 90 | Example | characteristic peaks, aliphatic integrations are |
| | 20⁴⁴; P229, | approximate: δ 10.37 (s, 1H), 8.74 (s, 2H), 8.40 (s, 1H), |
| | P239 | 7.92-7.81 (m, 2H), 7.75 (d, J = 8.7 Hz, 1H), 7.38-7.23 |
| | | (m, 4H), 7.18-7.10 (m, 1H), 4.74 (br d, J = 13.0 Hz, 2H), |
| | | 4.54-4.40 (m, 1H), 3.88-3.74 (m, 1H), 3.68-3.47 (m, |
| | | 3H), 3.16 (t, J = 6.4 Hz, 2H), 3.00-2.83 (m, 7H), 2.83- |
| | | 2.63 (m, 2H), 2.21 (s, 3H), 2.20-2.10 (m, 2H), 2.00- |
| | | 1.78 (m, 6H), 1.74-1.59 (m, 1H), 1.47-1.30 (m, 2H), |
| | | 1.29-1.13 (m, 2H); 879.3 |
| 91 | Example | characteristic peaks: δ 10.38 (s, 1H), 8.98 (s, 2H), 8.44 |
| | 20⁴⁵; P8, | (s, 1H), 8.16-8.08 (m, 1H), 7.97 (s, 1H), 7.79 (d, J = 8.7 |
| | P239 | Hz, 1H), 7.40-7.32 (m, 3H), 7.29-7.21 (m, 2H), 4.49 (t, |
| | | J = 5.7 Hz, 2H), 3.87-3.75 (m, 1H), 3.70-3.48 (m, 3H), |
| | | 3.12 (br d, J = 6.2 Hz, 2H), 2.84-2.64 (m, 4H), 2.21 (s, |
| | | 3H), 2.21-2.14 (m, 6H), 1.73-1.62 (m, 6H); 866.3 |
| 92 | Example | characteristic peaks, aliphatic integrations are |
| | 10⁴⁶; P229, | approximate: δ 11.09 (s, 1H), 8.50 (br s, 1H), 8.38 (s, |
| | P231 | 1H), 8.04-7.95 (m, 1H), 7.90 (br d, J = 8.9 Hz, 1H), 7.78 |
| | | (s, 1H), 7.73 (d, J = 8.7 Hz, 1H), 7.30 (d, J = 8.8 Hz, 1H), |
| | | 7.21-7.13 (m, 1H), 5.34 (dd, J = 12.8, 5.4 Hz, 1H), 4.53- |
| | | 4.40 (m, 1H), 3.60-3.51 (m, 4H), 3.33 (s, 3H), 3.17 (t, |
| | | J = 6.2 Hz, 2H), 2.96-2.84 (m, 1H), 2.77-2.61 (m, 3H), |
| | | 2.37 (t, J = 7.3 Hz, 2H), 2.21-2.11 (m, 2H), 2.05-1.76 |
| | | (m, 6H), 1.73-1.60 (m, 1H), 1.30-1.14 (m, 2H); 864.3 |
| 93 | Example | characteristic peaks, aliphatic integrations are |
| | 8⁴⁷; P2, | approximate: δ 11.08 (s, 1H), 8.88 (s, 1H), 8.23 (s, 1H), |
| | C136, P231 | 8.16 (s, 1H), 8.05-7.95 (m, 1H), 7.22-7.13 (m, 1H), |
| | | 7.07 (s, 1H), 6.95 (AB quartet, upfield doublet is |
| | | broadened, J_{AB} = 8.3 Hz, Δv_{AB} = 44.5 Hz, 2H), 6.67 (s, |
| | | 1H), 5.34 (dd, J = 12.7, 5.3 Hz, 1H), 4.54-4.41 (m, 1H), |
| | | 3.33 (s, 3H, assumed; partially obscured by water peak), |
| | | 3.22-3.11 (m, 2H), 2.96-2.83 (m, 1H), 2.78-2.61 (m, |
| | | 3H), 2.43-2.34 (m, 2H), 2.18-2.08 (m, 2H), 2.05-1.96 |
| | | (m, 1H), 1.96-1.75 (m, 6H), 1.73-1.59 (m, 1H), 1.29- |
| | | 1.13 (m, 2H); 788.3 |
| 94 | Example 17; | characteristic peaks, aliphatic integrations are |
| | P231, P251 | approximate: δ 11.11 (s, 1H), 8.16 (s, 1H), 7.47 (d, J = |
| | | 9.1 Hz, 1H), 7.08 (br s, 1H), 7.01 (d, half of AB quartet, J = |
| | | 8.1 Hz, 1H), 7.00-6.92 (m, 1H), 6.78 (br s, 1H), 5.34 |
| | | (dd, J = 12.7, 5.3 Hz, 1H), 3.33 (s, 3H, assumed; partially |
| | | obscured by water peak), 3.15-3.05 (m, 5H), 2.96- |
| | | 2.84 (m, 1H), 2.77-2.61 (m, 3H), 2.40-2.30 (m, 2H), |
| | | 2.15-2.05 (m, 6H), 2.04-1.93 (m, 2H), 1.86-1.74 (m, |
| | | 2H), 1.67-1.56 (m, 6H); 813.4 |
| 95 | Example 12; | characteristic peaks, aliphatic integrations are |
| | P16, P231 | approximate: δ 11.10 (s, 1H), 8.85 (s, 2H), 8.42 (s, 1H), |
| | | 8.24 (br t, J = 6 Hz, 1H), 7.90 (s, 1H), 7.76 (d, J = 8.7 Hz, |
| | | 1H), 7.33 (d, J = 9.3 Hz, 1H), 7.33-7.25 (m, 1H), 7.09 (s, |
| | | 1H), 6.99 (AB quartet, J_{AB} = 8.0 Hz, Δv_{AB} = 49.7 Hz, 2H), |
| | | 5.35 (dd, J = 12.7, 5.4 Hz, 1H), 3.12 (d, J = 6.3 Hz, 2H), |
| | | 2.97-2.84 (m, 1H), 2.76-2.62 (m, 4H), 2.23-2.11 (m, |
| | | 6H), 2.08-1.95 (m, 3H), 1.74-1.61 (m, 6H); 891.3 |
| 96 | P252, | characteristic peaks, aliphatic integrations are |
| | P239^{48,49} | approximate: δ 10.38 (s, 1H), 8.53 (s, 1H), 8.25 (br s, |
| | | 1H), 8.20-8.10 (m, 1H), 7.85 (d, J = 8.7 Hz, 1H), 7.61 |
| | | (br d, J = 8.8 Hz, 1H), 7.35 (d, half of AB quartet, J = 8.0 |
| | | Hz, 1H), 7.33 (d, J = 1.7 Hz, 1H), 7.26 (dd, component of |
| | | ABX system, J = 7.7, 1.7 Hz, 1H), 7.26-7.20 (m, 1H), |
| | | 4.59-4.47 (m, 1H), 3.81 (ddd, J = 12.3, 9.7, 5.3 Hz, 1H), |
| | | 3.69-3.48 (m, 3H), 3.17 (t, J = 6.3 Hz, 2H), 3.17-3.05 |
| | | (m, 1H), 2.85-2.64 (m, 2H), 2.22 (s, 3H), 2.22-2.13 (m, |
| | | 2H), 2.05-1.86 (m, 6H), 1.86-1.75 (m, 2H), 1.72-1.29 |
| | | (m, 7H); 853.3 |
| 97 | Example 12; | 11.40 (br s, 1H), 10.45 (s, 1H), 9.57 (br s, 1H), 8.57 (br s, |
| | P251, P241 | 1H), 8.26-8.19 (m, 2H), 8.00 (s, 1H), 7.58 (br d, J = 9.1 |
| | | Hz, 1H), 7.54 (d, J = 9.7 Hz, 1H), 7.28 (ddd, J = 11.0, 6.2, |
| | | 2.4 Hz, 1H), 7.21 (dd, J = 9.2, 1.4 Hz, 1H), 6.95-6.90 |
| | | (m, 2H), 3.88-3.77 (m, 2H), 3.77 (t, J = 6.8 Hz, 2H), |
| | | 3.69-3.56 (m, 2H), 3.25-3.13 (m, 4H), 3.10 (d, J = 6.2 |
| | | Hz, 2H), 3.03-2.89 (m, 2H), 2.77 (t, J = 6.7 Hz, 2H), |
| | | 2.71 (t, J = 7.3 Hz, 2H), 2.18-2.01 (m, 8H), 1.71-1.59 |
| | | (m, 6H); 784.2 |
| 98 | Example | characteristic peaks, aliphatic integrations are |
| | 9⁵⁰; P229, | approximate: δ 11.39 (br s, 1H), 11.08 (s, 1H), 9.71 (br s, |
| | P231 | 1H), 8.32 (t, J = 6 Hz, 1H), 8.19 (s, 1H), 7.52 (d, J = 8.9 |
| | | Hz, 1H), 7.29 (ddd, J = 11.1, 6.3, 2.3 Hz, 1H), 7.07 (br s, |
| | | 1H), 6.98 (AB quartet, upfield doublet is broadened, J_{AB} = |
| | | 8.1 Hz, Δv_{AB} = 50.6 Hz, 2H), 6.44 (dd, J = 8.9, 1.9 Hz, |
| | | 1H), 6.39 (br s, 1H), 5.35 (dd, J = 12.9, 5.4 Hz, 1H), 4.39- |
| | | 4.26 (m, 1H), 4.17-4.06 (m, 1H), 4.05-3.83 (m, 4H), |
| | | 3.33 (s, 3H), 3.16 (t, J = 6.4 Hz, 2H), 2.96-2.84 (m, 1H), |
| | | 2.78-2.56 (m, 4H), 2.17-1.94 (m, 5H), 1.94-1.77 (m, |
| | | 4H), 1.71-1.57 (m, 1H), 1.26-1.12 (m, 2H); 829.4 |
| 99 | Example 84; | 10.42 (s, 1H), 8.82 (s, 2H), 8.49 (br s, 1H), 8.47 (d, J = |
| | P232, P241 | 7.1 Hz, 1H), 8.17 (br s, 1H), 7.96 (s, 1H), 7.79 (br s, 1H), |
| | | 7.77-7.69 (m, 1H), 7.58 (s, 1H), 7.53 (br d, J = 9.1 Hz, |
| | | 1H), 7.20 (dd, J = 9.1, 1.2 Hz, 1H), 7.19-7.11 (m, 1H), |
| | | 7.17 (dd, J = 7.2, 1.7 Hz, 1H), 3.84-3.77 (m, 4H), 3.77 |
| | | (t, J = 6.7 Hz, 2H), 3.06 (d, J = 6.1 Hz, 2H), 2.77 (t, J = |
| | | 6.7 Hz, 2H), 2.66 (br t, J = 7.5 Hz, 2H), 2.48-2.42 (m, |
| | | 4H), 2.36 (br t, J = 7.2 Hz, 2H), 1.89-1.77 (m, 8H), 1.55- |
| | | 1.46 (m, 6H); 862.4 |
| 100 | Example | characteristic peaks, aliphatic integrations are |
| | 22⁵¹; P230, | approximate: δ 11.09 (br s, 1H), 8.40-8.22 (m, 4H), 7.87 |
| | P231 | (s, 1H), 7.61 (d, J = 8.7 Hz, 1H), 7.55 (s, 1H), 7.54-7.47 |
| | | (m, 1H), 7.42 (br d, J = 8.7 Hz, 1H), 7.09 (br s, 1H), 7.06- |
| | | 6.96 (m, 2H), 6.91 (br d, half of AB quartet, J = 8.1 Hz, |
| | | 1H), 5.34 (dd, J = 12.7, 5.4 Hz, 1H), 4.47-4.36 (m, 1H), |
| | | 4.05-3.96 (m, 2H), 3.65 (s, 2H), 3.33 (s, 3H), 3.19- |
| | | 3.10 (m, 2H), 2.96-2.81 (m, 3H), 2.77-2.56 (m, 3H), |
| | | 2.19-2.09 (m, 2H), 2.05-1.95 (m, 1H), 1.95-1.80 (m, |
| | | 5H), 1.72-1.58 (m, 1H), 1.27-1.11 (m, 2H); 824.3 |
| 101 | Example 22; | characteristic peaks, aliphatic integrations are |
| | P229, P231 | approximate: δ 11.08 (br s, 1H), 8.08 (s, 1H), 7.44 (d, J = |
| | | 9.1 Hz, 1H), 7.36-7.28 (m, 1H), 7.06 (br s, 1H), 7.00- |
| | | 6.92 (m, 1H), 6.94 (AB quartet, upfield doublet is |
| | | broadened, J_{AB} = 8.1 Hz, Δv_{AB} = 46.0 Hz, 2H), 6.57 (dd, J = |
| | | 9.1, 1.5 Hz, 1H), 6.28 (br s, 1H), 5.33 (dd, J = 12.9, 5.4 |
| | | Hz, 1H), 4.33-4.21 (m, 1H), 3.33 (s, 3H), 3.18-3.09 (m, |
| | | 2H), 3.04-2.83 (m, 3H), 2.15-2.05 (m, 2H), 2.05-1.94 |
| | | (m, 2H), 1.93-1.72 (m, 7H), 1.67-1.53 (m, 2H), 1.53- |
| | | 1.39 (m, 1H), 1.23-1.08 (m, 2H); 827.4 |
| 102 | Example 22; | characteristic peaks, aliphatic integrations are |
| | P229, P231 | approximate: δ 11.08 (br s, 1H), 8.22 (s, 1H), 8.13 (s, |
| | | 1H), 7.73-7.60 (m, 1H), 7.46 (d, J = 9.1 Hz, 1H), 7.11- |
| | | 7.01 (m, 1H), 7.04 (s, 1H), 6.93 (AB quartet, J_{AB} = 8.0 Hz, |
| | | Δv_{AB} = 51.1 Hz, 2H), 6.82 (br d, J = 9.2 Hz, 1H), 6.67 (br |
| | | s, 1H), 5.33 (dd, J = 12.7, 5.4 Hz, 1H), 4.37-4.25 (m, |
| | | 1H), 3.31 (s, 3H), 2.95-2.83 (m, 3H), 2.76-2.56 (m, |
| | | 8H), 2.35-2.26 (m, 2H), 2.14-2.05 (m, 2H), 2.04-1.95 |
| | | (m, 1H), 1.93-1.70 (m, 6H), 1.67-1.57 (m, 1H), 1.24- |
| | | 1.09 (m, 2H); 827.4 |
| 103 | Example | 10.38 (s, 1H), 8.33 (br t, J = 6 Hz, 1H), 8.22 (s, 1H), 7.55 |
| | 7⁵²; C152, | (d, J = 9.1 Hz, 1H), 7.33-7.26 (m, 2H), 7.29 (AB quartet, |
| | P239 | upfield doublet is broadened, J_{AB} = 7.8 Hz, Δv_{AB} = 37.8 |
| | | Hz, 2H), 6.93 (br d, J = 9.2 Hz, 1H), 6.89 (br s, 1H), 4.43- |
| | | 4.30 (m, 1H), 3.87-3.74 (m, 1H), 3.65-3.47 (m, 3H), |
| | | 3.16 (t, J = 6.3 Hz, 2H), 3.03-2.85 (m, 2H), 2.85-2.62 |
| | | (m, 2H), 2.21 (s, 3H), 2.16-2.06 (m, 2H), 1.95-1.80 (m, |
| | | 4H), 1.79-1.69 (m, 3H), 1.68-1.55 (m, 3H), 1.55-1.41 |
| | | (m, 2H), 1.38-1.06 (m, 10H); 883.4 |
| 104 | Example | characteristic peaks, aliphatic integrations are |
| | 20⁵³; P239 | approximate: δ 10.38 (s, 1H), 8.32 (br t, J = 6 Hz, 1H), |
| | | 8.23 (s, 1H), 7.59-7.54 (m, 1H), 7.45-7.39 (m, 1H), |
| | | 7.35 (t, J = 7.3 Hz, 1H), 7.29 (ddd, J = 11.0, 6.2, 2.3 Hz, |
| | | 1H), 6.99-6.87 (m, 2H), 4.44-4.31 (m, 1H), 3.87-3.75 |
| | | (m, 1H), 3.61-3.46 (m, 4H), 3.17 (t, J = 6.4 Hz, 2H), |
| | | 2.86-2.64 (m, 2H), 2.22 (s, 3H), 2.16-1.95 (m, 4H), |
| | | 1.95-1.75 (m, 6H), 1.75-1.52 (m, 4H), 1.27-1.13 (m, |
| | | 2H); 855.4 |
| 105 | Example | characteristic peaks: δ 10.43 (s, 1H), 8.85 (s, 2H), 8.56 |
| | 215³; P16, | (br s, 1H), 8.42 (s, 1H), 8.23 (br t, J = 6 Hz, 1H), 7.99 (s, |
| | C156, P246 | 1H), 7.89 (br s, 1H), 7.76 (d, J = 8.7 Hz, 1H), 7.58 (d, J = |
| | | 9.2 Hz, 1H), 7.33 (dd, J = 8.8, 1.6 Hz, 1H), 7.32-7.25 |
| | | (m, 1H), 7.20 (br d, J = 9.5 Hz, 1H), 3.76 (t, J = 6.7 Hz, |
| | | 2H), 3.12 (d, J = 6.3 Hz, 2H), 2.77 (t, J = 6.7 Hz, 2H), |
| | | 2.70 (br t, J = 7.3 Hz, 2H), 2.23-2.12 (m, 6H), 1.73- |
| | | 1.62 (m, 6H); 862.4 |
| 106 | Example 10; | 11.41 (br s, 1H), 11.11 (s, 1H), 10.74 (br s, 1H), 8.45 (s, |
| | C74, P240 | 1H), 8.35 (br t, J = 6 Hz, 1H), 7.87 (br s, 1H), 7.82-7.77 |
| | | (m, 2H), 7.76 (br d, J = 7.9 Hz, 1H), 7.52 (d, J = 8.0 Hz, |
| | | 1H), 7.35 (dd, J = 8.8, 1.5 Hz, 1H), 7.30 (ddd, J = 11.1, |
| | | 6.3, 2.3 Hz, 1H), 7.20 (d, J = 1.5 Hz, 1H), 7.11 (d, half of |
| | | AB quartet, J = 8.1 Hz, 1H), 7.02 (dd, component of ABX |
| | | system, J = 8.1, 1.6 Hz, 1H), 5.38 (dd, J = 12.8, 5.4 Hz, |
| | | 1H), 5.02-4.81 (m, 2H), 4.79-4.58 (m, 2H), 4.57-4.43 |
| | | (m, 1H), 3.77-3.65 (m, 2H), 3.36 (s, 3H), 3.18 (t, J = 6.3 |
| | | Hz, 2H), 3.14-3.05 (m, 2H), 2.98-2.85 (m, 1H), 2.79- |
| | | 2.58 (m, 2H), 2.22-2.12 (m, 2H), 2.05-1.85 (m, 5H), |
| | | 1.75-1.61 (m, 1H), 1.32-1.16 (m, 2H); 806.3 |
| 107 | Example 13; | characteristic peaks: δ 11.10 (br s, 1H), 8.75 (s, 1H), 8.43- |
| | C148, P231 | 8.37 (m, 2H), 8.37-8.28 (m, 2H), 8.17 (s, 1H), 7.71 |
| | | (AB quartet, J_{AB} = 8.8 Hz, Δv_{AB} = 17.9 Hz, 2H), 7.56- |
| | | 7.47 (m, 1H), 7.08 (s, 1H), 7.06-6.97 (m, 2H), 6.90 (d, |
| | | half of AB quartet, J = 8.0 Hz, 1H), 5.34 (dd, J = 12.8, 5.4 |
| | | Hz, 1H), 4.54-4.40 (m, 1H), 3.33 (s, 3H), 3.21-3.11 |
| | | (m, 2H), 2.97-2.83 (m, 1H), 2.78-2.54 (m, 5H, |
| | | assumed; partially obscured by solvent peak), 2.36 (br t, |
| | | J = 7.3 Hz, 2H), 2.22-2.10 (m, 2H), 2.07-1.73 (m, 7H), |
| | | 1.73-1.59 (m, 1H), 1.27-1.13 (m, 2H); 865.3 |
| 108 | Example 90; | characteristic peaks: δ 11.33 (v br s, 1H), 10.52 (s, 1H), |
| | P229, P249 | 8.74 (s, 2H), 8.40 (s, 1H), 8.37-8.30 (m, 1H), 7.83 (s, |
| | | 1H), 7.75 (d, J = 8.7 Hz, 1H), 7.66 (d, J = 8.2 Hz, 1H), |
| | | 7.58 (d, J = 1.9 Hz, 1H), 7.42 (br d, J = 8.2 Hz, 1H), 7.35- |
| | | 7.26 (m, 2H), 4.71 (d, J = 12.9 Hz, 2H), 4.53-4.42 (m, |
| | | 1H), 3.83-3.71 (m, 1H), 3.70-3.56 (m, 2H), 3.22-3.14 |
| | | (m, 2H), 3.14-3.04 (m, 2H), 2.99-2.88 (m, 2H), 2.79- |
| | | 2.71 (m, 2H), 2.21-2.11 (m, 2H), 2.01-1.75 (m, 6H), |
| | | 1.74-1.60 (m, 1H); 913.3 |
| 109 | Example | characteristic peaks, aliphatic integrations are |
| | 20⁵⁴; C141, | approximate: δ 10.38 (s, 1H), 8.51-8.42 (m, 1H), 8.17 |
| | P239 | (s, 1H), 7.65-7.53 (m, 2H), 7.49 (d, J = 9.0 Hz, 1H), 7.31 |
| | | (br s, 1H), 7.29 (AB quartet, J_{AB} = 7.8 Hz, Δv_{AB} = 36.4 Hz, |
| | | 2H), 6.90 (br d, J = 9.0 Hz, 1H), 6.78 (br s, 1H), 4.42- |
| | | 4.28 (m, 1H), 3.88-3.73 (m, 1H), 3.67-3.45 (m, 3H), |
| | | 3.22-3.02 (m, 5H), 2.89-2.62 (m, 5H), 2.21 (s, 3H), |
| | | 2.15-2.05 (m, 2H), 1.96-1.57 (m, 8H), 1.57-1.02 (m, |
| | | 10H); 851.4 |
| 110 | Example | characteristic peaks, aliphatic integrations are |
| | 109; P249 | approximate: δ 10.51 (s, 1H), 8.37 (br s, 1H), 8.20-8.14 |
| | | (m, 1H), 8.15 (s, 1H), 7.63 (d, half of AB quartet, J = 8.2 |
| | | Hz, 1H), 7.55 (d, J = 2.0 Hz, 1H), 7.47 (d, J = 9.2 Hz, 1H), |
| | | 7.44-7.36 (m, 3H), 6.88 (br d, J = 9.2 Hz, 1H), 6.75 (br |
| | | s, 1H), 4.41-4.26 (m, 1H), 3.82-3.70 (m, 1H), 3.13 (t, J = |
| | | 6.3 Hz, 2H), 3.10-3.03 (m, 4H), 2.79-2.71 (m, 2H), |
| | | 2.70-2.59 (m, 4H), 2.30-2.18 (m, 1H), 2.15-2.05 (m, |
| | | 2H), 1.94-1.71 (m, 6H), 1.70-1.57 (m, 3H), 1.57-1.32 |
| | | (m, 5H), 1.19-1.05 (m, 4H); 871.3 |
| 111 | Example | characteristic peaks, aliphatic integrations are |
| | 109; C152, | approximate: δ 10.36 (s, 1H), 8.28-8.21 (m, 1H), 8.19 |
| | P239 | (s, 1H), 7.51 (d, J = 9.1 Hz, 1H), 7.34 (d, half of AB |
| | | quartet, J = 8.0 Hz, 1H), 7.31 (br s, 1H), 7.30-7.22 (m, |
| | | 2H), 6.90 (br d, J = 9.1 Hz, 1H), 6.81 (br s, 1H), 4.42- |
| | | 4.29 (m, 1H), 3.88-3.73 (m, 1H), 3.01-2.84 (m, 4H), |
| | | 2.84-2.64 (m, 2H), 2.22 (s, 3H), 2.16-2.05 (m, 2H), |
| | | 1.97-1.70 (m, 7H), 1.70-1.58 (m, 1H), 1.58-1.04 (m, |
| | | 10H); 869.4 |
| 112 | Example 23; | characteristic peaks, aliphatic integrations are |
| | C20, P2, | approximate: δ 11.39 (br s, 1H), 10.39 (s, 1H), 8.43- |
| | P239 | 8.22 (m, 3H), 7.91 (s, 1H), 7.77 (s, 1H), 7.64 (d, J = 8.7 |
| | | Hz, 1H), 7.40-7.22 (m, 5H), 4.51-4.35 (m, 1H), 4.22- |
| | | 4.07 (m, 1H), 3.89-3.74 (m, 1H), 3.72-3.46 (m, 3H), |
| | | 3.24-3.10 (m, 2H), 2.87-2.62 (m, 2H), 2.22 (s, 3H), |
| | | 2.19-2.11 (m, 2H), 2.03-1.77 (m, 10H), 1.74-1.52 |
| | | (m, 3H), 1.48-1.12 (m, 7H); 851.4 |
| 113 | Example | characteristic peaks, aliphatic integrations are |
| | 20⁵⁵; C142, | approximate: δ 10.37 (s, 1H), 8.24 (s, 1H), 8.16 (s, 1H), |
| | P239 | 7.58-7.50 (m, 1H), 7.48 (d, J = 9.2 Hz, 1H), 7.34 (d, half |
| | | of AB quartet J = 7.9 Hz, 1H), 7.31 (d, J = 1.7 Hz, 1H), |
| | | 7.24 (dd, component of ABX system, J = 7.7, 1.8 Hz, 1H), |
| | | 7.03 (ddd, J = 13.0, 8.0, 2.1 Hz, 1H), 6.89 (dd, J = 9.1, |
| | | 1.9 Hz, 1H), 6.77 (br s, 1H), 4.39-4.27 (m, 1H), 3.87- |
| | | 3.75 (m, 1H), 3.14 (t, J = 6.3 Hz, 2H), 3.12-3.04 (m, |
| | | 4H), 2.83-2.68 (m, 2H), 2.21 (s, 3H), 2.15-2.05 (m, |
| | | 2H), 1.93-1.76 (m, 6H), 1.69-1.27 (m, 8H), 1.23-1.10 |
| | | (m, 2H); 855.3 |
| 114 | Example | characteristic peaks, aliphatic integrations are |
| | 20⁵⁶; P16, | approximate: δ 10.38 (s, 1H), 8.75 (s, 2H), 8.39 (s, 1H), |
| | P239 | 8.29 (s, 1H), 7.85 (br s, 1H), 7.73 (d, J = 8.7 Hz, 1H), 7.35 |
| | | (d, half of AB quartet, J = 7.9 Hz, 1H), 7.33-7.27 (m, |
| | | 3H), 7.25 (dd, component of ABX system, J = 7.7, 1.7 Hz, |
| | | 1H), 7.00 (ddd, J = 13.2, 8.1, 1.9 Hz, 1H), 4.57-4.34 (m, |
| | | 1H), 3.88-3.71 (m, 5H), 3.58-3.48 (m, 1H), 3.11 (d, J = |
| | | 6.2 Hz, 2H), 2.85-2.63 (m, 3H), 2.46-2.39 (m, 4H), |
| | | 2.22 (s, 3H), 2.20-2.11 (m, 7H), 1.93-1.77 (m, 2H), |
| | | 1.74-1.57 (m, 7H), 1.17-1.02 (m, 2H); 919.3 |
| 115 | Example | characteristic peaks, aliphatic integrations are |
| | 22⁵⁷; P230, | approximate: δ 8.46 (s, 1H), 8.42-8.19 (m, 2H), 8.07 (br |
| | P231 | s, 1H), 7.77 (d, J = 8.7 Hz, 1H), 7.58 (dd, J = 8.8, 1.5 Hz, |
| | | 1H), 7.57-7.50 (m, 1H), 7.08 (br s, 1H), 7.06-6.98 (m, |
| | | 2H), 6.91 (br d, half of AB quartet, J = 8.1 Hz, 1H), 5.34 |
| | | (dd, J = 12.6, 5.3 Hz, 1H), 4.55-4.43 (m, 1H), 3.72 (s, |
| | | 2H), 3.33 (s, 3H), 3.15 (t, J = 6.3 Hz, 2H), 2.97-2.80 (m, |
| | | 6H), 2.77-2.61 (m, 4H), 2.61-2.53 (m, 2H, assumed; |
| | | partially obscured by solvent peak), 2.21-2.11 (m, 2H), |
| | | 1.73-1.58 (m, 1H); 841.3 |
| 116 | Example 12; | characteristic peaks, aliphatic integrations are |
| | P230, P231 | approximate: δ 11.41 (br s, 1H), 11.10 (s, 1H), 10.86 (br |
| | | s, 1H), 8.90 (br s, 1H), 8.48 (s, 1H), 8.39-8.31 (m, 1H), |
| | | 8.19 (br s, 1H), 7.96 (s, 1H), 7.84 (d, J = 8.7 Hz, 1H), 7.37 |
| | | (dd, J = 8.7, 1.5 Hz, 1H), 7.30 (ddd, J = 11.0, 6.3, 2.3 Hz, |
| | | 1H), 7.12 (br s, 1H), 7.01 (AB quartet, upfield doublet is |
| | | broadened, J_{AB} = 8.0 Hz, Δv_{AB} = 44.1 Hz, 2H), 5.36 (dd, J = |
| | | 12.7, 5.5 Hz, 1H), 4.57-4.44 (m, 1H), 3.35 (s, 3H, |
| | | assumed; overlaps with water peak), 3.18 (t, J = 6.3 Hz, |
| | | 2H), 2.98-2.84 (m, 1H), 2.81-2.58 (m, 5H, assumed; |
| | | partially obscured by solvent peak), 2.22-2.13 (m, 2H), |
| | | 2.13-2.04 (m, 2H), 2.04-1.84 (m, 6H), 1.74-1.61 (m, |
| | | 1H); 821.3 |
| 117 | Example | characteristic peaks, aliphatic integrations are |
| | 13⁵⁸; P229, | approximate: δ 11.44 (br s, 1H), 11.10 (s, 1H), 10.16 (br |
| | P240 | s, 1H), 8.33 (br d, J = 6 Hz, 1H), 8.21 (s, 1H), 7.54 (d, J = |
| | | 8.9 Hz, 1H), 7.29 (ddd, J = 11.1, 6.3, 2.3 Hz, 1H), 7.12- |
| | | 7.08 (m, 2H), 6.95 (d, half of AB quartet, J = 8.1 Hz, 1H), |
| | | 6.46 (br d, J = 8.9 Hz, 1H), 6.41 (br s, 1H), 5.36 (dd, J = |
| | | 12.7, 5.4 Hz, 1H), 3.34 (s, 3H), 3.16 (t, J = 6.3 Hz, 2H), |
| | | 3.12-3.03 (m, 2H), 2.97-2.84 (m, 1H), 2.78-2.57 (m, |
| | | 2H), 2.17-2.06 (m, 2H), 2.05-1.94 (m, 2H), 1.94-1.77 |
| | | (m, 4H), 1.71-1.57 (m, 1H); 815.4 |
| 118 | Example 22; | characteristic peaks, aliphatic integrations are |
| | P229, P231 | approximate: δ 11.41 (br s, 1H), 11.10 (s, 1H), 10.30 (br |
| | | s, 1H), 8.33 (br t, J = 6 Hz, 1H), 8.15 (s, 1H), 7.50 (d, J = |
| | | 9.0 Hz, 1H), 7.29 (ddd, J = 11.0, 6.3, 2.4 Hz, 1H), 7.10- |
| | | 7.03 (m, 2H), 6.92 (br d, J = 8.4 Hz, 1H), 6.60 (br d, J = |
| | | 9.1 Hz, 1H), 6.34 (s, 1H), 5.36 (dd, J = 12.6, 5.4 Hz, 1H), |
| | | 4.37-4.25 (m, 1H), 3.34 (s, 2H), 3.21-3.12 (m, 4H), |
| | | 2.97-2.84 (m, 1H), 2.16-2.07 (m, 2H), 2.05-1.79 (m, |
| | | 7H), 1.71-1.57 (m, 1H), 1.25-1.13 (m, 2H); 813.4 |
| 119 | Example | characteristic peaks: δ 11.09 (s, 1H), 8.54 (s, 1H), 8.42 |
| | 22⁵⁹; P230, | (s, 1H), 8.25 (br s, 1H), 8.23 (s, 1H), 7.94 (s, 1H), 7.80 |
| | P231 | (dd, component of ABX system, J = 8.8, 1.4 Hz, 1H), 7.78- |
| | | 7.71 (m, 2H), 7.13-7.06 (m, 2H), 6.97 (AB quartet, |
| | | upfield doublet is broadened, J_{AB} = 8.1 Hz, Δv_{AB} = 41.0 |
| | | Hz, 2H), 5.35 (dd, J = 12.8, 5.4 Hz, 1H), 4.55-4.43 (m, |
| | | 1H), 3.94 (s, 4H), 3.34 (s, 3H), 3.16 (t, J = 6.3 Hz, 2H), |
| | | 2.98-2.83 (m, 1H), 2.79-2.57 (m, 6H), 2.23-2.12 (m, |
| | | 2H), 2.06-1.80 (m, 7H), 1.74-1.60 (m, 1H); 821.3 |
| 120 | Example | characteristic peaks, aliphatic integrations are |
| | 22⁶⁰; P232, | approximate: δ 11.38 (br s, 1H), 11.08 (s, 1H), 9.93 (br s, |
| | P231 | 1H), 8.83-8.77 (m, 1H), 8.78 (s, 2H), 8.48 (s, 1H), 8.24- |
| | | 8.08 (m, 3H), 7.28 (ddd, J = 10.9, 6.2, 2.3 Hz, 1H), 7.09 |
| | | (br s, 1H), 7.05 (d, half of AB quartet, J = 8.0 Hz, 1H), |
| | | 6.93 (dd, component of ABX system, J = 7.9, 1.6 Hz, 1H), |
| | | 5.41-5.29 (m, 1H), 4.28-4.11 (m, 2H), 3.34 (s, 3H), |
| | | 3.10 (d, J = 6.2 Hz, 2H), 2.97-2.84 (m, 1H), 2.12-1.95 |
| | | (m, 5H), 1.95-1.84 (m, 6H), 1.64-1.52 (m, 6H); 891.4 |
| 121 | C9, P231, | characteristic peaks, aliphatic integrations are |
| | P1^{61,62} | approximate: δ 11.07 (s, 1H), 8.64 (d, J = 4.9 Hz, 1H), |
| | | 8.32 (br s, 1H), 8.09 (br t, J = 6.3 Hz, 1H), 7.56-7.42 (m, |
| | | 2H), 7.26 (d, J = 4.8 Hz, 1H), 7.07 (br s, 1H), 7.00 (d, half |
| | | of AB quartet, J = 8.0 Hz, 1H), 6.92-6.87 (m, 1H), 5.33 |
| | | (dd, J = 12.7, 5.4 Hz, 1H), 3.86-3.76 (m, 4H), 3.33 (s, |
| | | 3H), 3.07 (d, J = 6.2 Hz, 2H), 2.97-2.83 (m, 1H), 2.48- |
| | | 2.41 (m, 4H), 2.38-2.30 (m, 2H), 2.05-1.95 (m, 1H), |
| | | 1.92-1.82 (m, 6H), 1.56-1.47 (m, 6H); 825.3 |
| 122 | Example | characteristic peaks, aliphatic integrations are |
| | 5⁶³; C72, | approximate: δ 11.09 (s, 1H), 8.43 (d, J = 5.1 Hz, 1H), |
| | P2, P231 | 8.19-8.10 (m, 2H), 7.29-7.23 (m, 1H), 7.24 (d, J = 5.0 |
| | | Hz, 1H), 7.08 (br s, 1H), 6.96 (AB quartet, upfield doublet |
| | | is broadened, J_{AB} = 8.1 Hz, Δv_{AB} = 46.4 Hz, 2H), 5.34 (dd, |
| | | J = 12.8, 5.4 Hz, 2H), 3.90-3.76 (m, 4H), 3.33 (s, 3H, |
| | | assumed; partially overlaps with water peak), 3.06 (d, J = |
| | | 6.2 Hz, 2H), 2.96-2.84 (m, 1H), 2.80 (s, 3H), 2.05-1.94 |
| | | (m, 2H), 1.60-1.48 (m, 6H); 872.4 |
| 123 | Example | 11.08 (s, 1H), 8.25 (s, 1H), 7.65-7.52 (m, 1H), 7.13- |
| | 21^{64,65}; P10, | 7.03 (m, 2H), 6.94 (AB quartet, J_{AB} = 7.9 Hz, Δv_{AB} = 46.0 |
| | P231 | Hz, 2H), 6.74 (s, 1H), 5.33 (dd, J = 12.6, 5.4 Hz, 1H), |
| | | 3.79-3.69 (m, 4H), 3.69-3.55 (m, 8H), 3.33 (s, 3H), |
| | | 3.07 (d, J = 6.1 Hz, 2H), 2.96-2.83 (m, 1H), 2.77-2.57 |
| | | (m, 4H), 2.46-2.37 (m, 4H), 2.37-2.28 (m, 2H), 2.05- |
| | | 1.95 (m, 1H), 1.94-1.82 (m, 6H), 1.82-1.73 (m, 2H), |
| | | 1.58-1.44 (m, 6H); 928.4 |
| 124 | Example | ¹H NMR (600 MHz, DMSO-d₆), characteristic peaks, |
| | 18⁶⁶; C59, | aliphatic integrations are approximate: δ 11.09 (s, 1H), |
| | P10, P231 | 10.99 (br s, 1H), 9.64 (br s, 1H), 8.73 (br s, 1H), 8.04 (t, J = |
| | | 6.3 Hz, 1H), 7.41 (br s, 1H), 7.29 (d, J = 11.3 Hz, 1H), |
| | | 7.08 (s, 1H), 7.04 (d, J = 8.0 Hz, 1H), 6.92 (d, J = 8.0 Hz, |
| | | 1H), 5.37-5.30 (m, 1H), 4.82-4.68 (m, 2H), 3.91 (s, |
| | | 3H), 3.11 (d, J = 6.3 Hz, 2H), 2.95-2.84 (m, 1H), 2.72- |
| | | 2.66 (m, 3H), 2.06-1.96 (m, 3H), 1.94-1.86 (m, 6H), |
| | | 1.58-1.50 (m, 6H); 855.8 |
| 125 | Example | characteristic peaks, integrations are approximate: δ |
| | 123; P10, | 11.08 (s, 1H), 8.80 (s, 1H), 8.24 (br s, 2H), 8.16 (br s, |
| | P231 | 1H), 7.65 (s, 1H), 7.50-7.40 (m, 1H), 7.15 (br s, 1H), |
| | | 7.11-6.97 (m, 3H), 6.93-6.88 (m, 1H), 5.34 (dd, J = |
| | | 12.5, 5.4 Hz, 1H), 3.95-3.82 (m, 4H), 3.33 (s, 3H, |
| | | assumed; overlaps with water peak), 3.08 (d, J = 6.4 Hz, |
| | | 2H), 2.96-2.84 (m, 1H), 2.06-1.73 (m, 10H), 1.56- |
| | | 1.49 (m, 6H); 909.4 |
| 126 | Example | ¹H NMR (600 MHz, DMSO-d₆), characteristic peaks: δ |
| | 8⁶⁷; P230, | 11.09 (s, 1H), 8.72 (s, 1H), 8.49 (s, 1H), 8.35 (br t, J = 6 |
| | P231 | Hz, 1H), 8.07 (d, J = 8.8 Hz, 1H), 7.81 (d, J = 8.8 Hz, 1H), |
| | | 7.30 (ddd, J = 11.1, 6.2, 2.2 Hz, 1H), 7.12 (s, 1H), 7.06 |
| | | (d, J = 8.0 Hz, 1H), 6.95 (br d, J = 8.1 Hz, 1H), 5.35 (dd, J = |
| | | 12.8, 5.1 Hz, 1H), 4.71 (v br s, 4H), 4.56-4.48 (m, 1H), |
| | | 4.17 (s, 3H), 3.18 (t, J = 6.3 Hz, 2H), 2.94-2.86 (m, 1H), |
| | | 2.77-2.68 (m, 3H), 2.66-2.60 (m, 1H), 2.22-2.15 (m, |
| | | 2H), 2.11-2.03 (m, 2H), 2.03-1.97 (m, 1H), 1.97-1.88 |
| | | (m, 4H), 1.73-1.64 (m, 1H), 1.29-1.19 (m, 2H); 852.6 |
| 127 | Example | 11.35 (br s, 1H), 11.07 (s, 1H), 9.85 (br s, 1H), 8.16 (br t, |
| | 123; P10, | J = 6.1 Hz, 1H), 7.27 (ddd, J = 11.0, 6.2, 2.3 Hz, 1H), |
| | P231 | 7.08 (br s, 1H), 7.05 (d, J = 8.0 Hz, 1H), 6.92 (br d, J = |
| | | 8.1 Hz, 1H), 6.57 (s, 1H), 5.35 (dd, J = 12.9, 5.4 Hz, 1H), |
| | | 4.78-4.67 (m, 2H), 3.65-3.48 (m, 4H), 3.48-3.39 (m, |
| | | 2H), 3.34 (s, 3H), 3.31-3.19 (m, 2H), 3.18-2.96 (m, |
| | | 6H), 2.96-2.84 (m, 1H), 2.77-2.58 (m, 4H), 2.10-1.81 |
| | | (m, 13H), 1.59-1.48 (m, 6H); 912.4 |
| 128 | Example | integrations are approximate: δ 11.08 (s, 1H), 8.23 (s, |
| | 123⁶⁸; P10, | 1H), 7.47-7.36 (m, 1H), 7.07 (br s, 1H), 7.07-7.00 (m, |
| | P231 | 1H), 7.01 (d, J = 7.9 Hz, 1H), 6.89 (br d, J = 8.1 Hz, 1H), |
| | | 6.69 (s, 1H), 5.34 (dd, J = 12.8, 5.4 Hz, 1H), 3.92 (s, 3H), |
| | | 3.85-3.75 (m, 4H), 3.33 (s, 3H, assumed; overlaps with |
| | | water peak), 3.06 (d, J = 6.2 Hz, 2H), 2.96-2.83 (m, 1H), |
| | | 2.77-2.57 (m, 4H), 2.48-2.41 (m, 4H), 2.38-2.30 (m, |
| | | 2H), 2.05-1.94 (m, 1H), 1.92-1.83 (m, 6H), 1.83-1.74 |
| | | (m, 2H), 1.56-1.45 (m, 6H); 873.3 |
| 129 | Example | ¹H NMR (600 MHz, DMSO-d₆), characteristic peaks: δ |
| | 18⁶⁹; C73, | 11.40 (br s, 1H), 11.10 (s, 1H), 10.49 (br s, 1H), 8.34 (br |
| | P231 | t, J = 6 Hz, 1H), 7.80 (s, 1H), 7.76-7.71 (m, 2H), 7.74 (s, |
| | | 1H), 7.48 (d, J = 7.9 Hz, 1H), 7.30 (ddd, J = 11.1, 6.3, 2.2 |
| | | Hz, 1H), 7.27 (dd, J = 8.8, 1.5 Hz, 1H), 7.11 (br s, 1H), |
| | | 7.06 (d, J = 8.0 Hz, 1H), 6.95 (br d, J = 8.1 Hz, 1H), 5.36 |
| | | (dd, J = 13.0, 5.4 Hz, 1H), 4.92-4.83 (m, 2H), 4.80- |
| | | 4.72 (m, 1H), 4.62-4.51 (m, 2H), 3.20 (t, J = 6.3 Hz, |
| | | 2H), 2.95-2.86 (m, 1H), 2.78-2.67 (m, 3H), 2.66-2.60 |
| | | (m, 2H), 2.24-2.17 (m, 1H), 2.10-1.97 (m, 7H), 1.97- |
| | | 1.91 (m, 2H), 1.73-1.63 (m, 1H), 1.32-1.21 (m, 3H), |
| | | 1.19-1.13 (m, 2H), 0.95-0.90 (m, 2H); 860.8 |
| 130 | Example | characteristic peaks, aliphatic integrations are |
| | 18⁷⁰; P232, | approximate: δ 11.41 (br s, 1H), 11.10 (s, 1H), 9.92 (br s, |
| | P231 | 1H), 9.03 (s, 1H), 8.81 (d, J = 7.3 Hz, 1H), 8.63 (s, 1H), |
| | | 8.28 (s, 1H), 8.22 (br t, J = 6 Hz, 1H), 8.13-8.07 (m, |
| | | 1H), 8.05 (s, 1H), 7.28 (ddd, J = 11.0, 6.3, 2.3 Hz, 1H), |
| | | 7.09 (s, 1H), 6.99 (AB quartet, J_{AB} = 8.1 Hz, Δv_{AB} = 50.3 |
| | | Hz, 2H), 5.41-5.28 (m, 1H), 4.70-4.55 (m, 2H), 3.34 |
| | | (s, 3H), 2.98-2.83 (m, 1H), 2.78-2.58 (m, 4H), 2.11- |
| | | 1.94 (m, 5H), 1.94-1.83 (m, 6H), 1.64-1.51 (m, 6H), |
| | | 1.50-1.39 (m, 1H); 891.3 |
| 131 | Example 17; | ¹H NMR (600 MHz, DMSO-d₆), characteristic peaks; δ |
| | P241, C74 | 10.81 (br s, 1H), 10.44 (s, 1H), 8.58 (s, 1H), 8.44 (s, 1H), |
| | | 8.38-8.33 (m, 1H), 8.00 (s, 1H), 7.85 (s, 1H), 7.79 (d, J = |
| | | 8.7 Hz, 1H), 7.75 (s, 1H), 7.73 (d, J = 8.2 Hz, 1H), 7.59 |
| | | (d, J = 9.1 Hz, 1H), 7.49 (d, J = 8.0 Hz, 1H), 7.34 (dd, J = |
| | | 8.7, 1.5 Hz, 1H), 7.32-7.28 (m, 1H), 4.96-4.83 (m, |
| | | 2H), 4.64-4.53 (m, 2H), 4.53-4.45 (m, 1H), 3.77 (t, J = |
| | | 6.7 Hz, 2H), 3.18 (t, J = 6.3 Hz, 2H), 2.77 (t, J = 6.7 Hz, |
| | | 2H), 2.20-2.14 (m, 2H), 2.14-2.06 (m, 2H), 1.97-1.87 |
| | | (m, 4H), 1.72-1.63 (m, 1H); 791.5 |
| 132 | Example | ¹H NMR (600 MHz, DMSO-d₆), aliphatic integrations are |
| | 17⁷¹; C61 | approximate: δ 11.39 (br s, 1H), 10.94 (s, 1H), 9.64 (br s, |
| | | 1H), 8.75 (d, J = 4.8 Hz, 1H), 8.19 (t, J = 6.5 Hz, 1H), |
| | | 7.51 (d, J = 8.4 Hz, 1H), 7.43 (d, J = 4.8 Hz, 1H), 7.30- |
| | | 7.24 (m, 1H), 7.08-7.04 (m, 2H), 5.04 (dd, J = 13.3, 5.2 |
| | | Hz, 1H), 4.81-4.71 (m, 2H), 4.25 (AB quartet, J_{AB} = 16.9 |
| | | Hz, ΔVAB = 69.7 Hz, 2H), 3.93-3.85 (m, 2H), 3.68-3.59 |
| | | (m, 2H), 3.25-3.16 (m, 2H), 3.14-3.03 (m, 4H), 2.94- |
| | | 2.86 (m, 1H), 2.86-2.79 (m, 2H), 2.63-2.55 (m, 2H), |
| | | 2.41-2.31 (m, 1H), 1.99-1.93 (m, 1H), 1.93-1.85 (m, |
| | | 6H), 1.80-1.73 (m, 2H), 1.69-1.62 (m, 2H), 1.61-1.49 |
| | | (m, 8H), 1.31-1.22 (m, 2H); 897.7 |
| 133 | C61^{72,73} | ¹H NMR (600 MHz, DMSO-d₆), characteristic peaks: δ |
| | | 11.42 (br s, 1H), 10.55 (s, 1H), 9.66 (br s, 1H), 8.74 (d, J = |
| | | 4.8 Hz, 1H), 8.19 (t, J = 6.4 Hz, 1H), 7.59 (d, J = 8.4 Hz, |
| | | 1H), 7.44 (br s, 1H), 7.42 (d, J = 4.8 Hz, 1H), 7.27 (ddd, J = |
| | | 11.0, 6.1, 2.2 Hz, 1H), 7.04 (dd, J = 8.4, 1.3 Hz, 1H), |
| | | 4.82-4.69 (m, 2H), 3.97 (s, 3H), 3.90 (t, J = 6.7 Hz, 2H), |
| | | 3.20-3.13 (m, 2H), 3.13-3.04 (m, 4H), 2.81 (t, J = 7.5 |
| | | Hz, 2H), 2.75 (t, J = 6.7 Hz, 2H), 2.12-2.05 (m, 2H), |
| | | 1.92-1.85 (m, 6H), 1.57-1.50 (m, 6H); 828.6 |
| 134 | Example | ¹H NMR (600 MHz, DMSO-d₆), characteristic peaks, |
| | 17⁷⁴; C66, | aliphatic integrations are approximate: δ 11.39 (br s, 1H), |
| | C61 | 11.10 (s, 1H), 9.07 (br s, 1H), 8.74 (br s, 1H), 8.19 (t, J = |
| | | 6.4 Hz, 1H), 7.45-7.36 (m, 1H), 7.27 (ddd, J = 10.9, 6.2, |
| | | 2.2 Hz, 1H), 7.05 (d, J = 8.0 Hz, 1H), 6.98 (s, 1H), 6.87 |
| | | (d, J = 8.0 Hz, 1H), 5.35 (dd, J = 12.9, 5.5 Hz, 1H), 4.66- |
| | | 4.48 (m, 2H), 3.80-3.57 (m, 4H), 3.21-3.11 (m, 2H), |
| | | 3.08 (d, J = 6.2 Hz, 2H), 2.95-2.83 (m, 1H), 2.75-2.59 |
| | | (m, 5H), 2.05-1.98 (m, 1H), 1.94-1.85 (m, 6H), 1.58- |
| | | 1.50 (m, 6H), 1.06 (br s, 6H); 871.6 |

1. Reaction of tert-butyl 4-(4,4,5,5-tetramethyl-1,3,2-dioxaborolan-2-yl)-1H-pyrazole-1-carboxylate with C20, mediated via tris(dibenzylideneacetone)dipalladium(0), 2-dicyclohexylphosphino-2′,4′,6′-triisopropylbiphenyl and sodium carbonate, afforded tert-butyl ({(1r,4r)-4-[6-(1H-pyrazol-4-yl)-2H-indazol-2-yl]cyclohexyl}methyl)carbamate. This material was deprotected with hydrogen chloride and then coupled to P1 using 1-[3-(dimethylamino)propyl]-3-ethylcarbodiimide hydrochloride and 1H-benzotriazol-1-ol, affording the requisite 3,5-difluoro-4-[(4-methoxyphenyl)methoxy]-N-({(1r,4r)-4-[6-(1H-pyrazol-4-yl)-2H-indazol-2-yl]cyclohexyl}methyl)benzamide.
2. Conversion of P229 and tert-butyl 1-oxa-4,9-diazaspiro[5.5]undecane-4-carboxylate to the requisite 2,3,5-trifluoro-4-[(4-methoxyphenyl)methoxy]-N-({(1r,4r)-4-[6-(1-oxa-4,9-diazaspiro[5.5]undecan-9-yl)-2H-indazol-2-yl]cyclohexyl}methyl)benzamide was carried out using the method described for synthesis of C142 from C23 in Example 6.
3. Oxidation of P246 to the requisite aldehyde was carried out with 1,1,1-tris(acetyloxy)-1,1-dihydro-1,2-benziodoxol-3-(1H)-one (Dess-Martin periodinane).
4. The requisite aldehyde may be prepared as described in footnote 3.
5. tert-Butyl 6-oxa-2,9-diazaspiro[4.5]decane-9-carboxylate may be separated into its component enantiomers via supercritical fluid chromatography {Column: Chiral Technologies Chiralpak IC, 30.0×250 mm, 5 μm; Mobile phase: 3:1 carbon dioxide/[methanol containing 0.2% (7 M ammonia in methanol)]; Back pressure: 100 bar; Flow rate: 80 mL/minute}. One of the two enantiomers was used to synthesize Example 30.
6. Reaction of P230 with tert-butyl 3-bromo-5,6-dihydroimidazo[1,2-a]pyrazine-7(8H)-carboxylate, [1,1′-bis(diphenylphosphino)ferrocene]dichloropalladium(II), and sodium carbonate provided the requisite tert-butyl 3-{2-[(1r,4r)-4-({2,3,5-trifluoro-4-[(4-methoxyphenyl)methoxy]benzamido}methyl)cyclohexyl]-2H-indazol-6-yl}-5,6-dihydroimidazo[1,2-a]pyrazine-7(8H)-carboxylate.
7. Reaction of P230 with tert-butyl 7-bromo-3,4-dihydroisoquinoline-2(1H)-carboxylate, [1,1′-bis(diphenylphosphino)ferrocene]dichloropalladium(II), and sodium carbonate provided the requisite tert-butyl 7-{2-[(1r,4r)-4-({2,3,5-trifluoro-4-[(4-methoxyphenyl)methoxy]benzamido}methyl)cyclohexyl]-2H-indazol-6-yl}-3,4-dihydroisoquinoline-2(1H)-carboxylate.
8. Reaction of C144 and tert-butyl 4-(2-hydroxyethyl)piperidine-1-carboxylate in the presence of potassium tert-butoxide, followed by hydrogen chloride-mediated protecting group removal from the product, afforded the requisite 2,3,5-trifluoro-4-hydroxy-N-{[4-(6-{2-[2-(piperidin-4-yl)ethoxy]pyrimidin-5-yl}-2H-indazol-2-yl)cyclohexyl]methyl}benzamide. In this case, the imine for the subsequent reductive amination was formed prior to addition of sodium triacetoxyborohydride, by combination of the secondary amine with P238 and 4-methylmorpholine at 65° C.
9. The requisite tert-butyl 4-(3-{[6-(N-hydroxycarbamimidoyl)pyridazin-3-yl]oxy}propyl)piperidine-1-carboxylate was prepared as follows: 6-chloropyridazine-3-carbonitrile was reacted with tert-butyl 4-(3-hydroxypropyl)piperidine-1-carboxylate and potassium tert-butoxide, and the resulting product was treated with hydroxylamine hydrochloride and triethylamine.
10. In this case, P9 was activated via reaction with 4-nitrophenyl carbonochloridate and triethylamine to provide 4-nitrophenyl 4-({2,3,5-trifluoro-4-[(4-methoxyphenyl)methoxy]benzamido}methyl)bicyclo[2.2.2]octane-1-carboxylate. The final deprotection was carried out with methanesulfonic acid.
11. Coupling of P1 with methyl 4-(aminomethyl)bicyclo[2.2.2]octane-1-carboxylate was carried out with O-(7-azabenzotriazol-1-yl)-N,N,N′N′-tetramethyluronium hexafluorophosphate and N,N-diisopropylethylamine. The resulting methyl 4-({3,5-difluoro-4-[(4-methoxyphenyl)methoxy]benzamido}methyl)bicyclo[2.2.2]octane-1-carboxylate was hydrolyzed with lithium hydroxide to afford the requisite 4-({3,5-difluoro-4-[(4-methoxyphenyl)methoxy]benzamido}methyl)bicyclo[2.2.2]octane-1-carboxylic acid.
12. In this case, the carboxylic acid and tert-butyl 4-(3-{[6-(N-hydroxycarbamimidoyl)pyridazin-3-yl]oxy}propyl)piperidine-1-carboxylate (see footnote 9) were coupled using 1-[3-(dimethylamino)propyl]-3-ethylcarbodiimide hydrochloride, 1-methyl-1H-imidazole, and 2-hydroxypyridine 1-oxide.
13. Reaction of C66 with tert-butyl (4-bromobutyl)carbamate, under the conditions described for conversion of C66 to C67 in Preparation P231, provided tert-butyl {4-[1-(2,6-dioxopiperidin-3-yl)-3-methyl-2-oxo-2,3-dihydro-1H-benzimidazol-5-yl]butyl}carbamate. Deprotection with hydrogen chloride yielded 3-[5-(4-aminobutyl)-3-methyl-2-oxo-2,3-dihydro-1H-benzimidazol-1-yl]piperidine-2,6-dione, which was reacted with C144 and triethylamine, then treated with trifluoroacetic acid, to afford Example 38.
14. Reaction of C66 with tert-butyl 6-bromohexanoate, under the conditions described for conversion of C66 to C67 in Preparation P231, followed by deprotection with trifluoroacetic acid, provided 6-[1-(2,6-dioxopiperidin-3-yl)-3-methyl-2-oxo-2,3-dihydro-1H-benzimidazol-5-yl]hexanoic acid. This material was treated with bis(pentafluorophenyl) carbonate and triethylamine to afford pentafluorophenyl 6-[1-(2,6-dioxopiperidin-3-yl)-3-methyl-2-oxo-2,3-dihydro-1H-benzimidazol-5-yl]hexanoate.
15. Reaction of C152 with pentafluorophenyl 6-[1-(2,6-dioxopiperidin-3-yl)-3-methyl-2-oxo-2,3-dihydro-1H-benzimidazol-5-yl]hexanoate (see footnote 14) in the presence of triethylamine, followed by deprotection with trifluoroacetic acid, afforded Example 39.
16. Reaction of P239 with 3-(piperidin-4-yl)propan-1-ol in the presence of triethylamine, followed by oxidation with 1,1,1-tris(acetyloxy)-1,1-dihydro-1,2-benziodoxol-3-(1H)-one (Dess-Martin periodinane), provided the requisite 3-{1-[3-(2,4-dioxo-1,3-diazinan-1-yl)-4-methylbenzoyl]piperidin-4-yl}propanal.
17. Reaction of C66 with tert-butyl 3-(2-bromoethyl)azetidine-1-carboxylate, using the method described for conversion of C66 to C67 in Preparation P231, was followed by deprotection with hydrogen chloride; this afforded the requisite 3-{5-[2-(azetidin-3-yl)ethyl]-3-methyl-2-oxo-2,3-dihydro-1H-benzimidazol-1-yl}piperidine-2,6-dione.
18. 3-{5-[2-(Azetidin-3-yl)ethyl]-3-methyl-2-oxo-2,3-dihydro-1H-benzimidazol-1-yl}piperidine-2,6-dione (see footnote 17) was reacted with C144; subsequent treatment with trifluoroacetic acid provided Example 41.
19. Reaction of tert-butyl 4-[(piperidin-4-yl)methyl]piperazine-1-carboxylate with C144 and potassium carbonate provided the requisite tert-butyl 4-{[1-(5-{2-[(1r,4r)-4-({2,3,5-trifluoro-4-[(4-methoxyphenyl)methoxy]benzamido}methyl)cyclohexyl]-2H-indazol-6-yl}pyrimidin-2-yl)piperidin-4-yl]methyl}piperazine-1-carboxylate.
20. Reaction of P229 with tert-butyl 4-(bromomethyl)-4-fluoropiperidine-1-carboxylate, under the conditions described for conversion of C66 to C67 in Preparation P231, provided tert-butyl 4-fluoro-4-({2-[(1r,4r)-4-({2,3,5-trifluoro-4-[(4-methoxyphenyl)methoxy]benzamido}methyl)cyclohexyl]-2H-indazol-6-yl}methyl)piperidine-1-carboxylate.
21. In this case, 4-methylmorpholine was used in the reductive amination.
22. Reaction of P239 with (piperidin-4-yl)methanol in the presence of triethylamine, followed by oxidation with 1,1,1-tris(acetyloxy)-1,1-dihydro-1,2-benziodoxol-3-(1H)-one (Dess-Martin periodinane), provided the requisite 1-[3-(2,4-dioxo-1,3-diazinan-1-yl)-4-methylbenzoyl]piperidine-4-carbaldehyde.
23. Reaction of P2 with C22, chloro-N,N,N′,N′-tetramethylformamidinium hexafluorophosphate, and 1-methyl-1H-imidazole provided N-{[(1r,4r)-4-(6-bromo-2H-indazol-2-yl)cyclohexyl]methyl}-2,3,5-trifluoro-4-[(4-methoxyphenyl)methoxy]benzamide. This material was coupled with tert-butyl 4-(6-chloropyridazin-3-yl)piperazine-1-carboxylate according to the method of D. A. Everson et al., Synlett 2014, 25, 233-238, and subsequently deprotected using hydrogen chloride to provide 2,3,5-trifluoro-4-hydroxy-N-{[(1r,4r)-4-{6-[6-(piperazin-1-yl)pyridazin-3-yl]-2H-indazol-2-yl}cyclohexyl]methyl}benzamide.
24. Reaction of 2,3,5-trifluoro-4-hydroxy-N-{[(1r,4r)-4-{6-[6-(piperazin-1-yl)pyridazin-3-yl]-2H-indazol-2-yl}cyclohexyl]methyl}benzamide (footnote 23) and 1-[3-(2,4-dioxo-1,3-diazinan-1-yl)-4-methylbenzoyl]piperidine-4-carbaldehyde (footnote 22) with sodium triacetoxyborohydride and 4-methylmorpholine afforded Example 49.
25. Conversion of C121 to 3-(2,4-dioxo-1,3-diazinan-1-yl)imidazo[1,2-a]pyridine-7-carbaldehyde was carried out using the method described in Preparation P245.
26. Analytical HPLC conditions. Column: Waters Atlantis dC18, 4.6×50 mm, 5 μm; Mobile phase A: water containing 0.05% trifluoroacetic acid (v/v); Mobile phase B: acetonitrile containing 0.05% trifluoroacetic acid (v/v); Gradient: 5.0% to 95% B, linear over 4.0 minutes, then 95% B for 1.0 minute; Flow rate: 2 mL/minute.
27. Reaction of C118 with 2-bromo-1,1-dimethoxyethane, using the method described for conversion of C66 to C67 in Preparation P231, was followed by deprotection with hydrogen chloride; this afforded the requisite [3-(2,4-dioxo-1,3-diazinan-1-yl)pyrazolo[1,5-a]pyridin-6-yl]acetaldehyde.
28. 2-(Bromomethyl)-1,3-dioxolane and C128 were coupled using the method of P. Zhang et al., J. Am. Chem. Soc. 2016, 138, 8084-8087; the product was deprotected with formic acid to afford the requisite [1-(2,6-dioxopiperidin-3-yl)-3-methyl-2-oxo-2,3-dihydro-1H-benzimidazol-4-yl]acetaldehyde.
29. Reaction of C144 with tert-butyl 4-[(piperidin-4-yl)oxy]piperidine-1-carboxylate and potassium carbonate provided the requisite tert-butyl 4-{[1-(5-{2-[(1r,4r)-4-({2,3,5-trifluoro-4-[(4-methoxyphenyl)methoxy]benzamido}methyl)cyclohexyl]-2H-indazol-6-yl}pyrimidin-2-yl)piperidin-4-yl]oxy}piperidine-1-carboxylate.
30. Analytical HPLC conditions. Column: Waters Sunfire C18, 4.6×50 mm, 5 μm; Mobile phase A: water containing 0.05% trifluoroacetic acid (v/v); Mobile phase B: acetonitrile containing 0.05% trifluoroacetic acid (v/v); Gradient: 20% to 60% B, linear over 3.75 minutes, then 60% B to 95% B over 0.25 minutes, then 95% B for 1.0 minute; Flow rate: 2 mL/minute.
31. Reaction of P239 with tert-butyl 1-oxa-4,9-diazaspiro[5.5]undecane-9-carboxylate and triethylamine, followed by hydrogen chloride deprotection of the product, afforded 1-[2-methyl-5-(1-oxa-4,9-diazaspiro[5.5]undecane-4-carbonyl)phenyl]-1,3-diazinane-2,4-dione. This material was treated with C144 and potassium carbonate; removal of the protecting group with hydrogen chloride afforded Example 62.
32. Reaction of P229 with tert-butyl 4-(bromomethyl)-3,3-difluoropiperidine-1-carboxylate, under the conditions described for conversion of C66 to C67 in Preparation P231, provided the requisite tert-butyl 3,3-difluoro-4-({2-[4-({2,3,5-trifluoro-4-[(4-methoxyphenyl)methoxy]benzamido}methyl)cyclohexyl]-2H-indazol-6-yl}methyl)piperidine-1-carboxylate.
33. Reaction of P229 with tert-butyl 9,9-difluoro-1-oxo-2,7-diazaspiro[4.5]decane-7-carboxylate in the presence of copper(I) iodide, cesium carbonate, and N¹,N¹-dimethylethane-1,2-diamine provided tert-butyl 9,9-difluoro-1-oxo-2-(2-{(1r,4r)-4-[(2,3,5-trifluoro-4-hydroxybenzamido)methyl]cyclohexyl}-2H-indazol-6-yl)-2,7-diazaspiro[4.5]decane-7-carboxylate. Deprotection of this material by treatment with methanesulfonic acid afforded the requisite N-({(1r,4r)-4-[6-(9,9-difluoro-1-oxo-2,7-diazaspiro[4.5]decan-2-yl)-2H-indazol-2-yl]cyclohexyl}methyl)-2,3,5-trifluoro-4-hydroxybenzamide.
34. Reaction of methyl 6-chloropyridazine-3-carboxylate with tert-butyl piperazine-1-carboxylate and cesium carbonate provided methyl 6-[4-(tert-butoxycarbonyl)piperazin-1-yl]pyridazine-3-carboxylate; hydrolysis with lithium hydroxide then afforded 6-[4-(tert-butoxycarbonyl)piperazin-1-yl]pyridazine-3-carboxylic acid. This material was reacted with P10, using the method described in Preparation P236 for the conversion of C103 to C104, to provide tert-butyl 4-(6-{3-[4-({2,3,5-trifluoro-4-[(4-methoxyphenyl)methoxy]benzamido}methyl)bicyclo[2.2.2]octan-1-yl]-1,2,4-oxadiazol-5-yl}pyridazin-3-yl)piperazine-1-carboxylate.
35. The final deprotection in this case was carried out with hydrogen chloride.
36. Reaction of C149, tert-butyl 3-oxo-1-oxa-8-azaspiro[4.5]decane-8-carboxylate, and sodium triacetoxyborohydride provided the requisite tert-butyl 3-[4-(5-{2-[(1r,4r)-4-({2,3,5-trifluoro-4-[(4-methoxyphenyl)methoxy]benzamido}methyl)cyclohexyl]-2H-indazol-6-yl}pyrazin-2-yl)piperazin-1-yl]-1-oxa-8-azaspiro[4.5]decane-8-carboxylate.
37. Separation of C126 into its component enantiomers was carried out via supercritical fluid chromatography {Column: Chiral Technologies Chiralcel OJ-H, 30×250 mm, 5 μm; Mobile phase: 4:1 carbon dioxide/[methanol containing 0.2% (7 M ammonia in methanol)]; Flow rate: 80 mL/minute; Back pressure: 100 bar}. The first-eluting enantiomer had a retention time of 4.24 minutes on analytical HPLC analysis [Column: Chiral Technologies Chiralcel OJ-H, 4.6×250 mm, 5 μm; Mobile phase A: carbon dioxide; Mobile phase B: methanol containing 0.2% (7 M ammonia in methanol); Gradient: 5% B for 0.50 minutes, then 5% to 100% B over 5.50 minutes; Flow rate: 3.0 mL/minute; Back pressure: 100 bar]. The second-eluting enantiomer exhibited a retention time of 5.12 minutes under the same conditions. The absolute configuration of these enantiomers was established via comparison with an enantiomerically pure sample derived from a compound reported in J. T. Kohrt et al., Org. Process Res. Dev. 2022, 26, 616-623. The second-eluting material was deprotected with hydrogen chloride, to provide 1-{2-methyl-5-[(3S)-3-(piperazin-1-yl)-1-oxa-8-azaspiro[4.5]decane-8-carbonyl]phenyl}-1,3-diazinane-2,4-dione, which was used in the synthesis of Example 73.
38. Alternatively, tert-butyl (3S)-3-amino-1-oxa-8-azaspiro[4.5]decane-8-carboxylate (see J. T. Kohrt et al., Org. Process Res. Dev. 2022, 26, 616-623) may be converted to tert-butyl (3S)-3-(piperazin-1-yl)-1-oxa-8-azaspiro[4.5]decane-8-carboxylate using the method described for its enantiomer in Preparation P247. This material may be used to synthesize Example 73 according to the method outlined for Example 68.
39. Reaction of P229 and tert-butyl 1-oxa-4,9-diazaspiro[5.5]undecane-4-carboxylate was mediated by (2-dicyclohexylphosphino-2′,6′-diisopropoxy-1,1′-biphenyl)[2-(2′-amino-1,1′-biphenyl)]palladium(II) methanesulfonate (RuPhos Pd G3) and cesium carbonate, affording tert-butyl 9-{2-[(1r,4r)-4-({2,3,5-trifluoro-4-[(4-methoxyphenyl)methoxy]benzamido}methyl)cyclohexyl]-2H-indazol-6-yl}-1-oxa-4,9-diazaspiro[5.5]undecane-4-carboxylate.
40. Coupling of P230 and 2,5-dibromopyrimidine was carried out with [1,1′-bis(diphenylphosphino)ferrocene]dichloropalladium(II) and sodium carbonate, to provide the requisite N-({(1r,4r)-4-[6-(5-bromopyrimidin-2-yl)-2H-indazol-2-yl]cyclohexyl}methyl)-2,3,5-trifluoro-4-[(4-methoxyphenyl)methoxy]benzamide.
41. Reaction of C66 with tert-butyl 2-bromo-7-azaspiro[3.5]nonane-7-carboxylate, using the method described for conversion of C66 to C67 in Preparation P231, was followed by deprotection with hydrogen chloride; this afforded 3-[5-(7-azaspiro[3.5]nonan-2-yl)-3-methyl-2-oxo-2,3-dihydro-1H-benzimidazol-1-yl]piperidine-2,6-dione. Subsequent reaction with C144 and potassium carbonate provided Example 80.
42. Reaction of P10 with 3-[4-(tert-butoxycarbonyl)piperazin-1-yl]benzoic acid was carried out using the method described in Preparation P236 for the conversion of C103 to P236, to afford the requisite 2,3,5-trifluoro-4-[(4-methoxyphenyl)methoxy]-N-[(4-{5-[3-(piperazin-1-yl)phenyl]-1,2,4-oxadiazol-3-yl}bicyclo[2.2.2]octan-1-yl)methyl]benzamide.
43. Reaction of C176 with benzyl 4-formylpiperidine-1-carboxylate and sodium triacetoxyborohydride, followed by deprotection of the product via treatment with hydrogen chloride, afforded the requisite 2,3,5-trifluoro-4-hydroxy-N-({(1r,4r)-4-[6-(2-{4-[(piperidin-4-yl)methyl]piperazin-1-yl}pyrimidin-5-yl)-2H-indazol-2-yl]cyclohexyl}methyl)benzamide.
44. Reaction of tert-butyl 4-(piperidin-4-yl)piperazine-1-carboxylate with (2-chloropyrimidin-5-yl)boronic acid, in the presence of potassium carbonate, provided (2-{4-[4-(tert-butoxycarbonyl)piperazin-1-yl]piperidin-1-yl}pyrimidin-5-yl)boronic acid; this material was coupled with P229 using [1,1′-bis(diphenylphosphino)ferrocene]dichloropalladium(II) and sodium carbonate to afford tert-butyl 4-[1-(5-{2-[(1r,4r)-4-({2,3,5-trifluoro-4-[(4-methoxyphenyl)methoxy]benzamido}methyl)cyclohexyl]-2H-indazol-6-yl}pyrimidin-2-yl)piperidin-4-yl]piperazine-1-carboxylate.
45. Coupling of 4-bromo-2-nitrobenzaldehyde and (2-chloropyrimidin-5-yl)boronic acid, mediated by tris(dibenzylideneacetone)dipalladium(0) and tri-tert-butylphosphonium tetrafluoroborate, provided 4-(2-chloropyrimidin-5-yl)-2-nitrobenzaldehyde. This material was subjected to imine formation with P8; subsequent cyclization via treatment with triethyl phosphite afforded N-({4-[6-(2-chloropyrimidin-5-yl)-2H-indazol-2-yl]bicyclo[2.2.2]octan-1-yl}methyl)-2,3,5-trifluoro-4-[(4-methoxyphenyl)methoxy]benzamide, which was reacted with tert-butyl 4-(2-hydroxyethyl)piperazine-1-carboxylate in the presence of (2-dicyclohexylphosphino-2′,6′-diisopropoxy-1,1′-biphenyl)[2-(2′-amino-1,1′-biphenyl)]palladium(II) methanesulfonate (RuPhos Pd G3) and cesium carbonate to provide the requisite tert-butyl 4-{2-[(5-{2-[4-({2,3,5-trifluoro-4-[(4-methoxyphenyl)methoxy]benzamido}methyl)bicyclo[2.2.2]octan-1-yl]-2H-indazol-6-yl}pyrimidin-2-yl)oxy]ethyl}piperazine-1-carboxylate.
46. Coupling of P229 with [6-(piperazin-1-yl)pyridin-3-yl]boronic acid was carried out using tris(dibenzylideneacetone)dipalladium(0), 2-dicyclohexylphosphino-2′,4′,6′-triisopropylbiphenyl (XPhos), and potassium carbonate to provide the requisite 2,3,5-trifluoro-4-[(4-methoxyphenyl)methoxy]-N-{[(1r,4r)-4-{6-[6-(piperazin-1-yl)pyridin-3-yl]-2H-indazol-2-yl}cyclohexyl]methyl}benzamide.
47. Reaction of P2 with C136, O-(7-azabenzotriazol-1-yl)-N,N,N,N′-tetramethyluronium hexafluorophosphate, and N,N-diisopropylethylamine provided N-{[(1r,4r)-4-(5-chloro-2H-pyrazolo[3,4-c]pyridin-2-yl)cyclohexyl]methyl}-2,3,5-trifluoro-4-[(4-methoxyphenyl)methoxy]benzamide. This was treated with tert-butyl piperazine-1-carboxylate, in the presence of tris(dibenzylideneacetone)dipalladium(0), 5-(di-tert-butylphosphino)-1′,3′,5′-triphenyl-1′H-[1,4′]bipyrazole, and potassium hydroxide, to afford tert-butyl 4-{2-[(1r,4r)-4-({2,3,5-trifluoro-4-[(4-methoxyphenyl)methoxy]benzamido}methyl)cyclohexyl]-2H-pyrazolo[3,4-c]pyridin-5-yl}piperazine-1-carboxylate.
48. Conversion of P252 to tert-butyl 9-(3-{2-[(1r,4r)-4-({2,3,5-trifluoro-4-[(4-methoxyphenyl)methoxy]benzamido}methyl)cyclohexyl]-2H-indazol-6-yl}-1,2,4-oxadiazol-5-yl)-3-azaspiro[5.5]undecane-3-carboxylate was carried out using the method described for synthesis of C28 from P4 in Preparation P18.
49. Removal of the tert-butoxycarbonyl group from tert-butyl 9-(3-{2-[(1r,4r)-4-({2,3,5-trifluoro-4-[(4-methoxyphenyl)methoxy]benzamido}methyl)cyclohexyl]-2H-indazol-6-yl}-1,2,4-oxadiazol-5-yl)-3-azaspiro[5.5]undecane-3-carboxylate (see footnote 48) was carried out using pyridine and trimethylsilyl trifluoromethanesulfonate; the product was reacted with P239 in the presence of triethylamine. Final deprotection with hydrogen chloride afforded Example 96.
50. The final deprotection in this case was carried out with trifluoroacetic acid.
51. Reaction of P230 with tert-butyl 2-bromo-5,6-dihydroimidazo[1,2-a]pyrazine-7(8H)-carboxylate, [1,1′-bis(diphenylphosphino)ferrocene]dichloropalladium(II), and sodium carbonate provided the requisite tert-butyl 2-{2-[(1r,4r)-4-({2,3,5-trifluoro-4-[(4-methoxyphenyl)methoxy]benzamido}methyl)cyclohexyl]-2H-indazol-6-yl}-5,6-dihydroimidazo[1,2-a]pyrazine-7(8H)-carboxylate.
52. Conversion of C152 to tert-butyl 9-[(4-{2-[(1r,4r)-4-({2,3,5-trifluoro-4-[(4-methoxyphenyl)methoxy]benzamido}methyl)cyclohexyl]-2H-indazol-6-yl}piperazin-1-yl)methyl]-3-azaspiro[5.5]undecane-3-carboxylate was carried out via the method described for synthesis of C178 from C152 in Example 20.
53. 2,3,5-Trifluoro-4-hydroxy-N-({(1r,4r)-4-[6-(piperazin-1-yl)-2H-indazol-2-yl]cyclohexyl}methyl)benzamide, which may be prepared by deprotection of C151 with hydrogen chloride, was reacted with tert-butyl 8-oxo-2-azaspiro[4.5]decane-2-carboxylate and sodium triacetoxyborohydride. The product was deprotected by treatment with trifluoroacetic acid, affording the requisite N-{[(1r,4r)-4-{6-[4-(2-azaspiro[4.5]decan-8-yl)piperazin-1-yl]-2H-indazol-2-yl}cyclohexyl]methyl}-2,3,5-trifluoro-4-hydroxybenzamide.
54. Deprotection of C141 with hydrogen chloride was followed by reaction with tert-butyl 9-oxo-3-azaspiro[5.5]undecane-3-carboxylate and sodium triacetoxyborohydride, affording tert-butyl 9-[4-(2-{(1r,4r)-4-[(3,5-difluoro-4-hydroxybenzamido)methyl]cyclohexyl}-2H-indazol-6-yl)piperazin-1-yl]-3-azaspiro[5.5]undecane-3-carboxylate.
55. Reaction of C142 with tert-butyl 2-oxo-8-azaspiro[4.5]decane-8-carboxylate and sodium triacetoxyborohydride, followed by deprotection with trifluoroacetic acid, provided the requisite N-{[(1r,4r)-4-{6-[4-(8-azaspiro[4.5]decan-2-yl)piperazin-1-yl]-2H-indazol-2-yl}cyclohexyl]methyl}-2,3,5-trifluoro-4-hydroxybenzamide.
56. Conversion of P16 to 2,3,5-trifluoro-4-[(4-methoxyphenyl)methoxy]-N-[(4-{6-[2-(piperazin-1-yl)pyrimidin-5-yl]-2H-indazol-2-yl}bicyclo[2.2.2]octan-1-yl)methyl]benzamide was carried out using the method described in Example 12 for synthesis of C158 from P232. Subsequent reaction with tert-butyl 4-formylpiperidine-1-carboxylate and sodium triacetoxyborohydride afforded the requisite tert-butyl 4-{[4-(5-{2-[4-({2,3,5-trifluoro-4-[(4-methoxyphenyl)methoxy]benzamido}methyl)bicyclo[2.2.2]octan-1-yl]-2H-indazol-6-yl}pyrimidin-2-yl)piperazin-1-yl]methyl}piperidine-1-carboxylate.
57. Reaction of P230 with tert-butyl 2-bromo-6,7-dihydro[1,3]thiazolo[5,4-c]pyridine-5(4H)-carboxylate, [1,1′-bis(diphenylphosphino)ferrocene]dichloropalladium(II), and sodium carbonate provided the requisite tert-butyl 2-{2-[(1r,4r)-4-({2,3,5-trifluoro-4-[(4-methoxyphenyl)methoxy]benzamido}methyl)cyclohexyl]-2H-indazol-6-yl}-6,7-dihydro[1,3]thiazolo[5,4-c]pyridine-5(4H)-carboxylate.
58. Conversion of P229 to the requisite tert-butyl 2-{2-[(1r,4r)-4-({2,3,5-trifluoro-4-[(4-methoxyphenyl)methoxy]benzamido}methyl)cyclohexyl]-2H-indazol-6-yl}-5-oxa-2,8-diazaspiro[3.5]nonane-8-carboxylate was carried out using the method described in Step 1 of Example 22.
59. In this case, the initial coupling with P230 was carried out using [1,1′-bis(diphenylphosphino)ferrocene]dichloropalladium(II) and sodium carbonate.
60. Reaction of P232 with 4,4,4′,4′,5,5,5′,5′-octamethyl-2,2′-bi-1,3,2-dioxaborolane and [1,1′-bis(diphenylphosphino)ferrocene]dichloropalladium(II) provided the boron derivative; this was coupled with 5-bromo-2-iodopyrimidine in the presence of [1,1′-bis(diphenylphosphino)ferrocene]dichloropalladium(II) and sodium carbonate to afford the requisite N-({4-[7-(5-bromopyrimidin-2-yl)imidazo[1,2-a]pyridin-2-yl]bicyclo[2.2.2]octan-1-yl}methyl)-2,3,5-trifluoro-4-[(4-methoxyphenyl)methoxy]benzamide.
61. Treatment of C9 with hydroxylamine hydrochloride and N,N-diisopropylethylamine provided the N-hydroxycarbamimidoyl derivative, which was reacted with 2-chloropyrimidine-4-carboxylic acid, O-(7-azabenzotriazol-1-yl)-N,N,N′N′-tetramethyluronium hexafluorophosphate, and N,N-diisopropylethylamine. The resulting material was cyclized by exposure to tetrabutylammonium fluoride to provide tert-butyl ({4-[5-(2-chloropyrimidin-4-yl)-1,2,4-oxadiazol-3-yl]bicyclo[2.2.2]octan-1-yl}methyl)carbamate. Reaction with 1-(piperazin-1-yl)ethan-1-one, followed by amide cleavage with potassium hydroxide in ethanol, afforded the requisite tert-butyl [(4-{5-[2-(piperazin-1-yl)pyrimidin-4-yl]-1,2,4-oxadiazol-3-yl}bicyclo[2.2.2]octan-1-yl)methyl]carbamate.
62. Reaction of tert-butyl [(4-{5-[2-(piperazin-1-yl)pyrimidin-4-yl]-1,2,4-oxadiazol-3-yl}bicyclo[2.2.2]octan-1-yl)methyl]carbamate (see footnote 61) with P231, sodium triacetoxyborohydride, and N,N-diisopropylethylamine, followed by hydrogen chloride-mediated protecting group removal, provided 3-(5-{3-[4-(4-{3-[4-(aminomethyl)bicyclo[2.2.2]octan-1-yl]-1,2,4-oxadiazol-5-yl}pyrimidin-2-yl)piperazin-1-yl]propyl}-3-methyl-2-oxo-2,3-dihydro-1H-benzimidazol-1-yl)piperidine-2,6-dione. Amide formation with P1, using O-(7-azabenzotriazol-1-yl)-N,N,N′,N′-tetramethyluronium hexafluorophosphate and N,N-diisopropylethylamine, and subsequent treatment with hydrogen chloride, afforded Example 121.
63. Reaction of 2-(methylsulfanyl)pyrimidine-4-carboxylic acid and N-methoxymethanamine, in the presence of O-(7-azabenzotriazol-1-yl)-N,N,N′,N′-tetramethyluronium hexafluorophosphate and N,N-diisopropylethylamine, provided N-methoxy-N-methyl-2-(methylsulfanyl)pyrimidine-4-carboxamide. This material was treated with ethylmagnesium bromide, and subsequently brominated to give 2-bromo-1-[2-(methylsulfanyl)pyrimidin-4-yl]propan-1-one; this was reacted with C72 at elevated temperature to afford the requisite tert-butyl [(4-{5-methyl-4-[2-(methylsulfanyl)pyrimidin-4-yl]-1,3-thiazol-2-yl}bicyclo[2.2.2]octan-1-yl)methyl]carbamate.
64. Reaction of methyl 2,6-dichloropyrimidine-4-carboxylate with morpholine in the presence of N,N-diisopropylethylamine, followed by similar reaction with tert-butyl piperazine-1-carboxylate, provided the requisite methyl 2-[4-(tert-butoxycarbonyl)piperazin-1-yl]-6-(morpholin-4-yl)pyrimidine-4-carboxylate.
65. Conversion of carboxylic acid methyl 2-[4-(tert-butoxycarbonyl)piperazin-1-yl]-6-(morpholin-4-yl)pyrimidine-4-carboxylate (see footnote 64) to the requisite 2,3,5-trifluoro-4-hydroxy-N-[(4-{5-[6-(morpholin-4-yl)-2-(piperazin-1-yl)pyrimidin-4-yl]-1,2,4-oxadiazol-3-yl}bicyclo[2.2.2]octan-1-yl)methyl]benzamide was carried out using the method described for synthesis of C34 from P20 in Preparation P20.
66. Reaction of P10 with C59, O-(7-azabenzotriazol-1-yl)-N,N,N′,N′-tetramethyluronium hexafluorophosphate, and N,N-diisopropylethylamine, followed by cyclization with tetrabutylammonium fluoride, provided tert-butyl 4-(4-{3-[4-({2,3,5-trifluoro-4-[(4-methoxyphenyl)methoxy]benzamido}methyl)bicyclo[2.2.2]octan-1-yl]-1,2,4-oxadiazol-5-yl}pyrimidin-2-yl)piperazine-1-carboxylate. Exposure to potassium tert-butoxide in methanol afforded the requisite tert-butyl 4-(4-{3-[4-({3,5-difluoro-2-methoxy-4-[(4-methoxyphenyl)methoxy]benzamido}methyl)bicyclo[2.2.2]octan-1-yl]-1,2,4-oxadiazol-5-yl}pyrimidin-2-yl)piperazine-1-carboxylate.
67. Reaction of tert-butyl 2-amino-4-chloro-5,7-dihydro-6H-pyrrolo[3,4-d]pyrimidine-6-carboxylate with diiodomethane and 3-methylbutyl nitrite provided tert-butyl 4-chloro-2-iodo-5,7-dihydro-6H-pyrrolo[3,4-d]pyrimidine-6-carboxylate, which was treated with sodium methoxide to give tert-butyl 2-iodo-4-methoxy-5,7-dihydro-6H-pyrrolo[3,4-d]pyrimidine-6-carboxylate. Coupling of this material with P230 using chloro[(di(1-adamantyl)-N-butylphosphine)-2-(2-aminobiphenyl)]palladium(II) and tripotassium phosphate afforded the requisite tert-butyl 4-methoxy-2-{2-[(1r,4r)-4-({2,3,5-trifluoro-4-[(4-methoxyphenyl)methoxy]benzamido}methyl)cyclohexyl]-2H-indazol-6-yl}-5,7-dihydro-6H-pyrrolo[3,4-d]pyrimidine-6-carboxylate.
68. Reaction of methyl 2-chloro-6-methoxypyrimidine-4-carboxylate with tert-butyl piperazine-1-carboxylate in the presence of potassium carbonate, followed by hydrolysis of the resulting ester with sodium hydroxide, afforded 2-[4-(tert-butoxycarbonyl)piperazin-1-yl]-6-methoxypyrimidine-4-carboxylic acid.
69. Bromination of C73, via treatment with N-bromosuccinimide and acetic acid, provided tert-butyl 5-{3-bromo-2-[(1r,4r)-4-({2,3,5-trifluoro-4-[(4-methoxyphenyl)methoxy]benzamido}methyl)cyclohexyl]-2H-indazol-6-yl}-1,3-dihydro-2H-isoindole-2-carboxylate, which was coupled with 2-cyclopropyl-4,4,5,5-tetramethyl-1,3,2-dioxaborolane in the presence of [1,1′-bis(diphenylphosphino)ferrocene]dichloropalladium(II) and tripotassium phosphate to afford the requisite tert-butyl 5-{3-cyclopropyl-2-[(1r,4r)-4-({2,3,5-trifluoro-4-[(4-methoxyphenyl)methoxy]benzamido}methyl)cyclohexyl]-2H-indazol-6-yl}-1,3-dihydro-2H-isoindole-2-carboxylate.
70. Reaction of P232 with 4,4,4′,4′,5,5,5′,5′-octamethyl-2,2′-bi-1,3,2-dioxaborolane, [1,1′-bis(diphenylphosphino)ferrocene]dichloropalladium(II), and potassium acetate provided the boron intermediate, which was coupled with tert-butyl 4-(5-bromopyrazin-2-yl)piperazine-1-carboxylate, in the presence of mesyl[(tri-t-butylphosphine)-2-(2-aminobiphenyl)]palladium(II) [P(t-Bu)₃Pd G3] and tripotassium phosphate, to afford the requisite tert-butyl 4-(5-{2-[4-({2,3,5-trifluoro-4-[(4-methoxyphenyl)methoxy]benzamido}methyl)bicyclo[2.2.2]octan-1-yl]imidazo[1,2-a]pyridin-7-yl}pyrazin-2-yl)piperazine-1-carboxylate.
71. Reaction of 3-(5-bromo-1-oxo-1,3-dihydro-2H-isoindol-2-yl)piperidine-2,6-dione and 4-(2-{[tert-butyl(dimethyl)silyl]oxy}ethyl)piperidine was mediated via dichloro[1,3-bis(2,6-di-3-pentylphenyl)imidazol-2-ylidene](3-chloropyridyl)palladium(II) (Pd-PEPPSI™-iPENT). The resulting material was deprotected with tetrabutylammonium fluoride to provide 3-{5-[4-(2-hydroxyethyl)piperidin-1-yl]-1-oxo-1,3-dihydro-2H-isoindol-2-yl}piperidine-2,6-dione. This material was oxidized with tetrapropylammonium perruthenate and 4-methylmorpholine N-oxide to afford the requisite {1-[2-(2,6-dioxopiperidin-3-yl)-1-oxo-2,3-dihydro-1H-isoindol-5-yl]piperidin-4-yl}acetaldehyde.
72. Reaction of 6-bromo-1-methyl-1H-indazol-3-amine and prop-2-enoic acid with hydrochloric acid, in the presence of tetrabutylammonium bromide, provided N-(6-bromo-1-methyl-1H-indazol-3-yl)-s-alanine. This material was treated with sodium cyanate and acetic acid, followed by hydrochloric acid, to yield 1-(6-bromo-1-methyl-1H-indazol-3-yl)-1,3-diazinane-2,4-dione, which was subjected to a coupling with 3-bromo-1,1-dimethoxypropane using the conditions described in Preparation P231 for conversion of C66 to C67. Subsequent acetal hydrolysis with formic acid afforded the requisite 3-[3-(2,4-dioxo-1,3-diazinan-1-yl)-1-methyl-1H-indazol-6-yl]propanal.
73. Deprotection of C61 with hydrogen chloride afforded 2,3,5-trifluoro-4-hydroxy-N-[(4-{5-[2-(piperazin-1-yl)pyrimidin-4-yl]-1,2,4-oxadiazol-3-yl}bicyclo[2.2.2]octan-1-yl)methyl]benzamide. This material was reacted with 3-[3-(2,4-dioxo-1,3-diazinan-1-yl)-1-methyl-1H-indazol-6-yl]propanal (see footnote 72), sodium triacetoxyborohydride, triethylamine, and acetic acid to provide Example 133.
74. Reaction of C66 and 3-bromo-2,2-dimethylpropan-1-ol, using the conditions described in Preparation P231 for conversion of C66 to C67, provided 3-[5-(3-hydroxy-2,2-dimethylpropyl)-3-methyl-2-oxo-2,3-dihydro-1H-benzimidazol-1-yl]piperidine-2,6-dione. This material was oxidized via treatment with Dess-Martin periodinane [1,1,1-tris(acetyloxy)-1,1-dihydro-1,2-benziodoxol-3-(1H)-one] to afford the requisite 3-[1-(2,6-dioxopiperidin-3-yl)-3-methyl-2-oxo-2,3-dihydro-1H-benzimidazol-5-yl]-2,2-dimethylpropanal.

The following protocols may of course be varied by those skilled in the art.

### A. Whole-Cell Huh7-HSD17B13 HiBiT Assay

Degrader potency and maximal efficacy of test compounds were determined using a whole-cell Huh7-HSD17B13 HiBiT assay, utilizing a luminescence readout. A HiBiT tag was fused to the N-terminus of HSD17β13, and therefore the HiBiT luminescence signal produced in the assay was proportional to the level of HiBiT-HSD17β13 fusion protein in a sample.

A Huh7 HiBiT-HSD17β13 OE cell line was generated using electroporation with an N-terminal HiBiT-tagged HSD17β13 isoform D overexpression plasmid, followed by geneticin selection to produce a stable pool. Clones were created with varying HiBiT signals to optimize HiBiT HSD17 β13 ratios. A clone with similar HSD17β13 expression levels as human primary hepatocytes was selected for the compound screen. A counter screen was also conducted using a Cell Titer-Glo (CTG) luminescent assay that generated a luminescent signal proportional to the amount of ATP present, which showed the number of viable cells present in a culture.

Greiner 384-well white opaque, flat bottom, tissue culture treated plates (Greiner #781080) were used for both HiBiT and CTG assays. The treated plates were spotted with 25 nL of a compound which had been serial diluted 1 in 3.162 in 100% DMSO for an 11-point concentration response curve, with duplicate points at each concentration. The maximum concentration of the compounds was 1 mM, which was later diluted with cell suspension for a maximum concentration of 1 μM). High percentage effect (HPE) wells and zero percentage effect (ZPE) wells located in columns 1 and 24 were spotted with 25 nL of DMSO (Sigma #D2650) using an ECHO Acoustic Liquid Handler (Labcyte Echo 550 Series). Each plate was performed in duplicate.

The Huh7 HiBiT-HSD17β13 OE cells were plated on top of a compound or a DMSO control at 3,000 viable cells/well in 25 μL growth media (DMEM (Gibco #10569), 10% FBS HI (Gibco #16140), 1× Pen/Strep (Gibco #15070), 1×MEM Non-Essential Amino Acids (Gibco #11140), and 300 μg/mL Geneticin (Gibco #10131) in columns 2-24 of the plates. Huh7 parental cells were plated using the same parameters in column 1 (HPE) in the same culture medium, excluding Geneticin selection. Cells were incubated overnight with the compounds for 24 hours (with lid) at 37° C. (95% O₂: 6% CO₂).

Each assay was conducted after a 24 hour incubation period. In the HiBiT assay plates, 25 μL of complete Nano-Glo HiBiT Lytic detection system (Promega #PRN3040), consisting of HiBiT Lytic Buffer with the addition of 1:100 LgBiT Protein and 1:50 Substrate, was added to each well. In CTG counter screen plates, 25 μL of Cell Titer-Glo Luminescent Reagent (Promega #G7573) was added to each well. Both plates were centrifuged for 2 minutes at 1000 rpm and incubated at room temperature, in the dark, for 10 minutes. Then Luminescence was read using an EnVision Plate Reader (Perkin Elmer PE2103).

The raw data from the Envision Plate Reader was expressed as relative luminescence units (RLUs) for each well of the assay plates. Using HPE and ZPE controls, the % effect was calculated for each sample well as follows: Percent effect=[(sample RLU−HPE RLU)\(ZPE RLU−HPE RLU)]*100. The percent effect was plotted versus the compound concentration and a 50% degradation (0050) was determined using a four-parameter logistic dose response equation. The raw data was analyzed using ABase (IDBS).

Table 5 summarizes the assay data obtained for protein degrader compounds described in the Examples detailed above.

TABLE 5

| | | Huh7_HIBIT_{—} | |
|---|---|---|---|
| | Huh7_HIBIT_{—} | HSD17β13_DC₅₀ | Huh7_HIBIT_{—} |
| | HSD17β13_DC₅₀ | GMean Asymptote | HSD17β13_DC₅₀ |
| Ex. | GMean DC₅₀ (nM) | Maximum (%) | Count |
| 1 | 0.11* | 87* | 5** |
| 2 | 0.16* | 83* | 8** |
| 3 | 0.19* | 84* | 7** |
| 4 | 0.38 | 84 | 2 |
| 5 | 0.31 | 82 | 4 |
| 6 | 1.4* | 76* | 63** |
| 7 | 3.0* | 78* | 4** |
| 8 | 0.21* | 87* | 8** |
| 9 | 0.26* | 83* | 34** |
| 10 | 0.28 | 82 | 2 |
| 11 | 0.94 | 81 | 2 |
| 12 | 0.14 | 84 | 6 |
| 13 | 0.14 | 81 | 6 |
| 14 | 0.23 | 85 | 2 |
| 15 | 0.31 | 85 | 4 |
| 16 | 0.42 | 82 | 4 |
| 17 | 0.47 | 85 | 2 |
| 18 | 0.58 | 82 | 2 |
| 19 | 0.73 | 82 | 2 |
| 20 | 2.3 | 78 | 2 |
| 21 | 0.38 | 74 | 1 |
| 22 | 1.6 | 77 | 1 |
| 23 | 2.4 | 75 | 4 |
| 24 | 0.18* | 88* | 6** |
| 25 | 0.85 | 84 | 2 |
| 26 | 0.50 | 82 | 7 |
| 27 | 0.82 | 86 | 4 |
| 28 | 0.59 | 90 | 2 |
| 29 | 1.9 | 80 | 2 |
| 30 | 1.7 | 74 | 2 |
| 31 | 0.99 | 85 | 2 |
| 32 | 0.30 | 83 | 6 |
| 33 | 0.62 | 86 | 6 |
| 34 | 0.23 | 81 | 4 |
| 35 | 2.7 | 87 | 4 |
| 36 | 2.2 | 86 | 2 |
| 37 | 2.0 | 85 | 2 |
| 38 | 2.6 | 79 | 3 |
| 39 | 1.1 | 86 | 4 |
| 40 | 4.1 | 74 | 2 |
| 41 | 0.86 | 81 | 2 |
| 42 | 3.3 | 83 | 4 |
| 43 | 0.29 | 83 | 2 |
| 44 | 1.5 | 84 | 3 |
| 45 | 3.1 | 76 | 4 |
| 46 | 0.57 | 84 | 6 |
| 47 | 4.3 | 78 | 3 |
| 48 | 1.5 | 74 | 2 |
| 49 | 3.7 | 82 | 2 |
| 50 | 0.91 | 85 | 2 |
| 51 | 2.1 | 73 | 2 |
| 52 | 1.6 | 80 | 2 |
| 53 | 0.78 | 77 | 4 |
| 54 | 0.64 | 83 | 4 |
| 55 | 0.49 | 82 | 4 |
| 56 | 0.64 | 82 | 4 |
| 57 | 4.8 | 76 | 2 |
| 58 | 1.5 | 73 | 4 |
| 59 | 1.0 | 80 | 4 |
| 60 | 0.65 | 78 | 4 |
| 61 | 2.6 | 72 | 4 |
| 62 | 4.5 | 70 | 2 |
| 63 | 2.6 | 80 | 2 |
| 64 | 0.51 | 85 | 2 |
| 65 | 3.8 | 81 | 2 |
| 66 | 0.32 | 75 | 2 |
| 67 | 0.18 | 89 | 2 |
| 68 | 1.7 | 79 | 4 |
| 69 | 0.16 | 88 | 2 |
| 70 | 1.1 | 85 | 2 |
| 71 | 2.4 | 76 | 2 |
| 72 | 1.6 | 78 | 2 |
| 73 | 0.96 | 76 | 2 |
| 74 | 0.23 | 87 | 2 |
| 75 | 0.19 | 88 | 4 |
| 76 | 0.15* | 89* | 4** |
| 77 | 0.93 | 86 | 2 |
| 78 | 0.47 | 78 | 6 |
| 79 | 0.31 | 78 | 4 |
| 80 | 1.4 | 84 | 6 |
| 81 | 0.86 | 81 | 2 |
| 82 | 2.0 | 74 | 4 |
| 83 | 0.50 | 81 | 2 |
| 84 | 0.59 | 83 | 2 |
| 85 | 0.86 | 73 | 1 |
| 86 | 0.27 | 78 | 1 |
| 87 | 0.50 | 78 | 1 |
| 88 | 1.5 | 81 | 1 |
| 89 | 3.0 | 77 | 2 |
| 90 | 6.1* | 60* | 5** |
| 91 | 3.2 | 73 | 2 |
| 92 | 0.40 | 87 | 2 |
| 93 | 0.69 | 83 | 2 |
| 94 | 0.21 | 84 | 2 |
| 95 | 0.24 | 80 | 2 |
| 96 | 2.7 | 81 | 2 |
| 97 | 2.4 | 76 | 2 |
| 98 | 0.82 | 78 | 3 |
| 99 | 1.7 | 70 | 3 |
| 100 | 2.2 | 84 | 2 |
| 101 | 0.88 | 74 | 1 |
| 102 | 1.5 | 74 | 1 |
| 103 | 1.2 | 76 | 7 |
| 104 | 3.6 | 78 | 2 |
| 105 | 1.3 | 76 | 6 |
| 106 | 0.22 | 80 | 2 |
| 107 | 0.56 | 86 | 2 |
| 108 | 1.3 | 80 | 2 |
| 109 | 1.1 | 81 | 4 |
| 110 | 2.9 | 77 | 2 |
| 111 | 0.85 | 80 | 4 |
| 112 | 0.97 | 81 | 4 |
| 113 | 1.4 | 79 | 2 |
| 114 | 2.6 | 77 | 2 |
| 115 | 0.26 | 79 | 3 |
| 116 | 0.20 | 88 | 2 |
| 117 | 0.95 | 79 | 2 |
| 118 | 2.9 | 78 | 1 |
| 119 | 0.28 | 75 | 1 |
| 120 | 0.15 | 82 | 2 |
| 121 | 0.20 | 85 | 1 |
| 122 | 0.23 | 73 | 2 |
| 123 | 0.23 | 79 | 1 |
| 124 | 0.27 | 72 | 2 |
| 125 | 0.28 | 85 | 1 |
| 126 | 0.36 | 84 | 1 |
| 127 | 0.37 | 79 | 1 |
| 128 | 0.40 | 79 | 2 |
| 129 | 0.42 | 83 | 1 |
| 130 | 0.50 | 83 | 1 |
| 131 | 0.87 | 78 | 1 |
| 132 | 1.6 | 82 | 1 |
| 133 | 2.1 | 73 | 2 |
| 134 | 6.5 | 85 | 1 |

*Value represents the geometric mean for all forms tested of the compound (parent and salt forms)
**Number of times any form of the compound was tested (parent or salt form)

### Embodiments

The following non-limiting embodiments provide illustrative examples of the invention, but do not limit the scope of the invention.

Embodiment 1. A compound of Formula II:

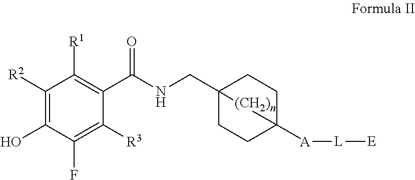

wherein:
A is —NH—C(O)—, —C(O)—, or heteroaryl, wherein heteroaryl has 1, 2, 3, or 4 heteroatoms selected from O, N, and S, and wherein A is optionally substituted with one or two R⁴;
R¹, R², and R³ are each independently selected from H and fluoro;
R⁴ is selected from oxo, hydroxyl, chloro, fluoro, (C₁-C₆)alkyl, (C₁-C₆)alkoxy, (C₁-C₆)fluoroalkyl, (C₃-C₆)cycloalkyl, and heterocyclyl, wherein the heterocyclyl has 1, 2, or 3 heteroatoms selected from O and N;
n is 0, 1, or 2;
L is a linker; and
E is an E3 ubiquitin ligase binder,
or a pharmaceutically acceptable salt thereof.

Embodiment 2. The compound of embodiment 1, wherein the compound has the Formula IIA:

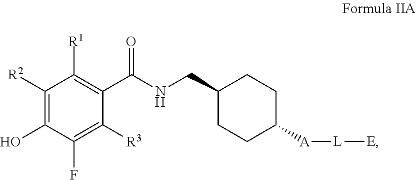

or a pharmaceutically acceptable salt thereof.

Embodiment 3. The compound of embodiment 1, wherein the compound has the Formula IIB:

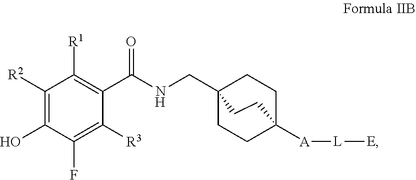

or a pharmaceutically acceptable salt thereof.

Embodiment 4. The compound of any one of embodiments 1-3, wherein A is thiazolyl, pyrazolyl, oxazolyl, imidazolyl, isoxazolyl, isothiazolyl, imidazotriazinyl, imidazopyridazinyl, imidazopyridinyl, benzoimidazolyl, benzothiazolyl, purinyl, pyridopyridazinyl, quinazolinyl, indazolyl, imidazopyridinyl, benzooxazolyl, pyrazolopyridinyl, isoindolinonyl, triazolyl, or oxadiazolyl, or a pharmaceutically acceptable salt thereof.

Embodiment 5. The compound of any one of embodiments 1-3, wherein A is:

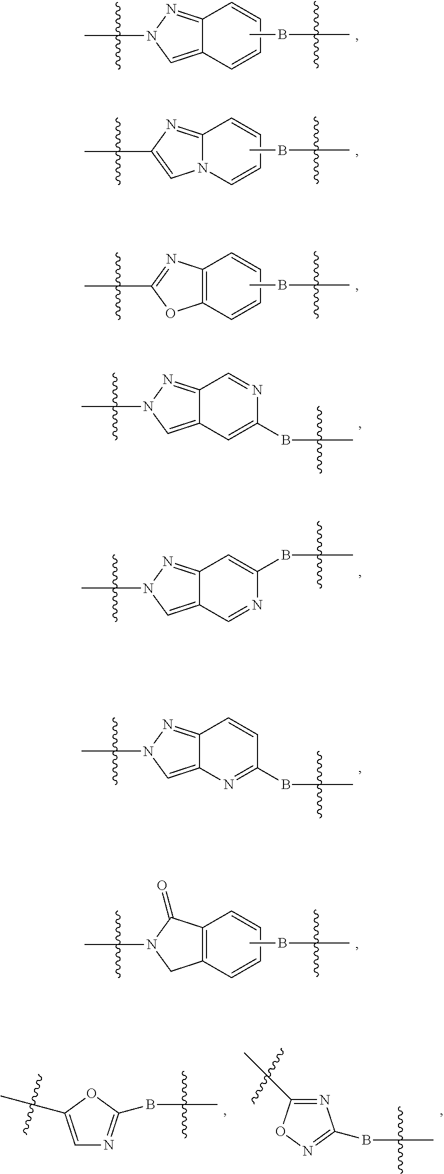

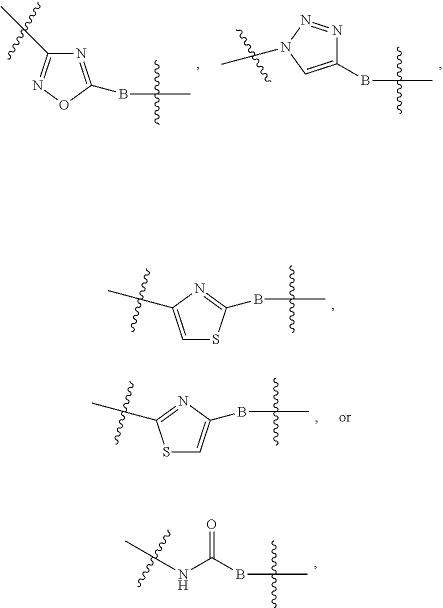

or a pharmaceutically acceptable salt thereof.

Embodiment 6. The compound of any one of embodiments 1-5, wherein A is indazolyl.

Embodiment 7. The compound of any one of embodiments 1-5, wherein A is oxadiazolyl.

Embodiment 8. The compound of any one of embodiments 1-7, wherein at least one of R¹, R², and R³ are fluoro; or a pharmaceutically acceptable salt thereof.

Embodiment 9. The compound of any one of embodiments 1-8, wherein L is of the formula:

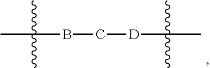

wherein:
B is absent; or aryl, heteroaryl, heterocyclyl, —C(O)—, (C₁-C₆)alkylene, (C₃-C₆)cycloalkylene, (C₁-C₆)fluoroalkylene, (C₁-C₆)alkoxy, or (C₁-C₆)fluoroalkoxy, wherein the heteroaryl, or heterocyclyl has 1, 2, or 3 heteroatoms selected from O, N, and S, and wherein B is optionally substituted with one or two R⁵;
C is absent; or —NH—C(O)—R⁷, —S(O)₂—R⁷, —O—S(O)₂—R⁷, —C(O)—, (C₁-C₆)alkylene, (C₁-C₆)aminoalkylene, (C₃-C₆)cycloalkylene, (C₁-C₆)alkoxy, (C₃-C₆)cycloether, (C₁-C₆)fluoroalkylene, (C₁-C₆)fluoroalkoxy, aryl, heteroaryl, or heterocyclyl, wherein the heteroaryl or heterocyclyl has 1, 2, or 3 heteroatoms selected from O, N, and S, and wherein C is optionally substituted with one, two or three R⁶;
D is (C₁-C₆)alkylene, (C₁-C₆)aminoalkylene, —NH(C₁-C₆)alkylene, (C₁-C₆)alkoxy, —C(O)—, aryl, heteroaryl, heterocyclyl, (C₀-C₆)alkylene-heterocyclyl-C(O)—, —C(O)—(C₁-C₆)alkylene, heterocyclyl-(C₁-C₆)alkylene-aryl-(C₁-C₆)alkoxy, (C₁-C₆)heterocyclyl-(C₁-C₆)heterocyclyl-C(O)—, (C₀-C₂)alkylene-aryl-(C₁-C₆)alkoxy, —O-heterocyclyl-C(O)—, (C₁-C₆)cycloalkyl-(C₁-C₆)heterocyclyl, wherein the heteroaryl or heterocyclyl has 1, 2, or 3 heteroatoms selected from O, N, and S, wherein D is optionally substituted with one or two R⁸; or a bond;
R⁵, R⁶, and R⁸ are each independently selected from oxo, hydroxyl, halogen, (C₁-C₆)alkyl, (C₁-C₆)alkoxy, (C₁-C₆)fluoroalkyl, (C₃-C₆)cycloalkyl, heteroaryl, and heterocyclyl, wherein the heterocyclyl has 1, 2, or 3 heteroatoms selected from O and N;
R⁷ is (C₁-C₆)alkyl, (C₁-C₆)alkoxy, (C₁-C₆)fluoroalkyl, or (C₃-C₆)cycloalkyl; or a pharmaceutically acceptable salt thereof.

Embodiment 10. The compound of embodiment 9, wherein B is absent, or a pharmaceutically acceptable salt thereof.

Embodiment 11. The compound of embodiment 9, wherein B is pyridinyl, pyrimidinyl, pyridazinyl, pyrazinyl, pyrazolyl, piperazinyl, quinoxalinyl, phenyl, triazolyl, thiazolyl, thiadiazolyl, oxazolyl, imidazolyl, indazolyl, (C₁-C₆)alkylene, (C₁-C₆)fluoroalkylene, (C₁-C₆)alkoxy, pyrrolopyridinyl, isoindolinyl, isoquinolinyl, tetrahydroisoquinolinyl, thiazolopyridinyl, tetrahydrothiazolopyridinyl, imidazopyrazinyl, tetrahydroimidazopyrazinyl, pyrazolopyrazinyl, tetrahydropyrazolopyrazinyl, phenyl, oxadiazaspirodecanyl, diazaspirooctanyl, or diazaspirodecan-1-only, wherein B is optionally substituted with one or two halogen, oxo, hydroxyl, (C₁-C₆)alkyl, (C₃-C₆)cycloalkyl, (C₁-C₆)fluoroalkyl, (C₁-C₆)alkoxy, or (C₃-C₆)cycloether, or a pharmaceutically acceptable salt thereof.

Embodiment 12. The compound of embodiment 9, wherein B is (C₁-C₆)alkylene, (C₁-C₆)heteroalkylene, (C₁-C₆)alkoxy, phenyl, isoindolinyl, pyrimidinyl, pyridazinyl, pyrazolyl, 6,7-dihydro-5H-pyrrolo[3,4-b]pyridinyl, tetrahydroisoquinolinyl, tetrahydrothiazolo[5,4-c]pyridinyl, tetrahydroimidazo[1,2-a]pyrazinyl, 6-oxa-2,9-diazaspiro[4.5]decanyl, 2,6-diazaspiro[3.4]octanyl, 7-diazaspiro[4.5]decan-1-onyl, or tetrahydropyrazolo[1,5-a]pyrazinyl, or a pharmaceutically acceptable salt thereof.

Embodiment 13. The compound of any one of embodiments 9-12, wherein C is absent, or a pharmaceutically acceptable salt thereof.

Embodiment 14. The compound of any one of embodiments 9-12, wherein C is (C₁-C₃)alkylene, (C₁-C₆)aminoalkylene, (C₁-C₆)alkoxy, pyridinyl, oxolanyl, (C₃-C₆)cycloalkyl, (C₁-C₆)fluoroalkylene, —C(O)—, piperazinyl, piperidinyl, azetidinyl, azaspiroundecanyl, azaspirononanyl, azaspiroundecanyl, diazaspirooctanyl, diazaspirodecanyl, diazaspirononanyl, diazaspirododecanyl, diazaspiroundecanyl, oxadiazaspirononanyl, oxadiazaspiroundecanyl, oxa-azaspirodecanyl, decahydronaphthyridinyl, octahydropyrrolopyridinyl, or octahydropyridopyrazinyl; wherein C is optionally substituted with one, two or three halogen, oxo, hydroxyl, (C₁-C₆)alkyl, (C₃-C₆)cycloalkyl, (C₁-C₆)fluoroalkyl, or (C₁-C₆)alkoxy, or a pharmaceutically acceptable salt thereof.

Embodiment 15. The compound of any one of embodiments 9-12, wherein C is (C₁-C₆)alkylene, (C₁-C₆)aminoalkylene, (C₁-C₆)alkoxy, piperazinyl, piperidinyl, azetidinyl, —C(O)—, 5-oxa-diazaspiro[3.5]nonanyl, 1-oxa-diazaspiro[5.5]undecanyl, 3-azaspiro[5.5]undecanyl, 1-oxa-8-azaspiro[4.5]decanyl, 8-azaspiro[4.5]decanyl, 7-azaspiro[3.5]nonanyl, 2,8-diazaspiro[4.5]decanyl, 1-oxa-4,9-diazaspiro[5.5]undecanyl, 3,9-diazaspiro[5.5]undecanyl, 1-oxa-8-azaspiro[4.5]decanyl, 3-azaspiro[5.5]undecanyl, 2-azaspiro[5.5]undecanyl, 2,6-diazaspiro[3.4]octanyl, 3,9-diazaspiro[5.6]dodecanyl, 2,7-diazaspiro[3.5]nonanyl, 2,9-diazaspiro[5.5]undecanyl, decahydro-1,5-naphthyridinyl, octahydro-1H-pyrrolo[3,4-c]pyridinyl, 2,6-diazaspiro[3.5]nonanyl, 2-azaspiro[3.5]nonanyl, octahydro-1H-pyrrolo[3,2-c]pyridinyl, octahydro-2H-pyrido[1,2-a]pyrazinyl, or a pharmaceutically acceptable salt thereof.

Embodiment 16. The compound of any one of embodiments 1-15, wherein D is (C₁-C₆)alkylene, (C₁-C₆)aminoalkylene, (C₁-C₆)alkoxy, —C(O)—, (C₀-C₆)alkylene-heterocyclyl-C(O)—, —C(O)—(C₁-C₆)alkylene, heterocyclyl-(C₁-C₆)alkylene-aryl-(C₁-C₆)alkoxy, (C₁-C₆)heterocyclyl-(C₁-C₆)heterocyclyl-C(O)—, (C₀-C₂)alkylene-aryl-(C₁-C₆)alkoxy, —O-heterocyclyl-C(O)—, (C₁-C₆)cycloalkyl-(C₁-C₆)heterocyclyl; or a bond, or a pharmaceutically acceptable salt thereof.

Embodiment 17. The compound of any one of embodiments 1-16, wherein D is methylene, ethylene, or propylene, or a pharmaceutically acceptable salt thereof.

Embodiment 18. The compound of any one of embodiments 1-16, wherein D is heterocyclyl-C(O)—, or a pharmaceutically acceptable salt thereof.

Embodiment 19. The compound of any one of embodiments 1-16, wherein D is —C(O)—(C₁-C₆)alkylene, or a pharmaceutically acceptable salt thereof.

Embodiment 20. The compound of any one of embodiments 1-16, wherein D is —C(O)—, or a pharmaceutically acceptable salt thereof.

Embodiment 21. The compound of any one of embodiments 9-20, wherein: A is heteroaryl; B is heteroaryl; C is heterocyclyl; and D is (C₁-C₃)alkylene, or a pharmaceutically acceptable salt thereof.

Embodiment 22. The compound of any one of embodiments 9-20, wherein: A is indazolyl or oxadiazolyl; B is pyrimidinyl; C is piperazinyl; and D is methylene, ethylene, or propylene, or a pharmaceutically acceptable salt thereof.

Embodiment 23. The compound of any one of embodiments 1-22, wherein E comprises a benzimidazolinone, a dihydropyrimidine-dione, or a thalidomide; or a pharmaceutically acceptable salt thereof.

Embodiment 24. The compound of any one of embodiments 1-23, wherein E is of Formula II-IIIa or Formula II-IIIab:

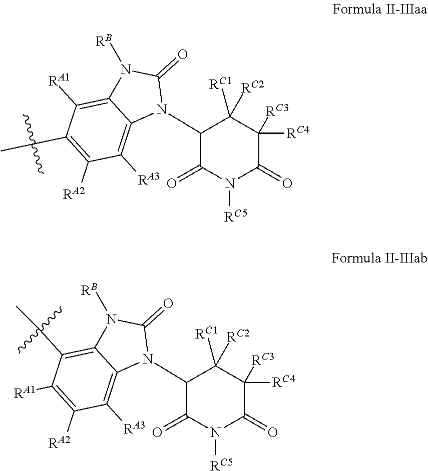

wherein:
R^{A1}, R^{A2}, and R^{A3} are each independently H, hydroxyl, halogen, (C₁-C₆)alkyl, (C₁-C₆)heteroalkyl, (C₁-C₆)alkoxy, or NH₂;
R^{B} is H or (C₁-C₆)alkyl; and R^{C1}, R^{C2}, R^{C3}, R^{C4}, and R^{C5} are each independently H, hydroxyl, halogen, or (C₁-C₆)alkyl, (C₁-C₆)heteroalkyl, (C₁-C₆)alkoxy, or NH₂;

or a pharmaceutically acceptable salt thereof.

Embodiment 25. The compound of embodiment 24, wherein:
R^{A1}, R^{A2}, and R^{A3} are each independently H;
R^{B} is (C₁-C₃)alkyl;
R^{C1}, R^{C2}, R^{C3}, R^{C4} are each independently H; and
R^{C5} is H;

or a pharmaceutically acceptable salt thereof.

Embodiment 26. The compound of any one of embodiments 1-25, wherein E is

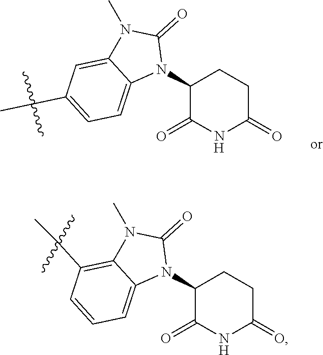

or a pharmaceutically acceptable salt thereof.

Embodiment 27. The compound of any one of embodiments 1-25, wherein E is

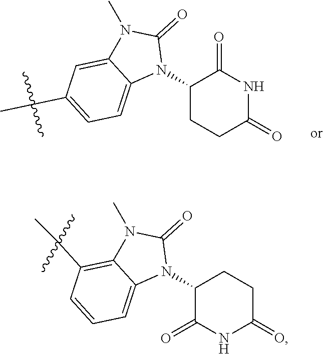

or a pharmaceutically acceptable salt thereof.

Embodiment 28. The compound of any one of embodiments 1-23, wherein E is of Formula II-IIIb:

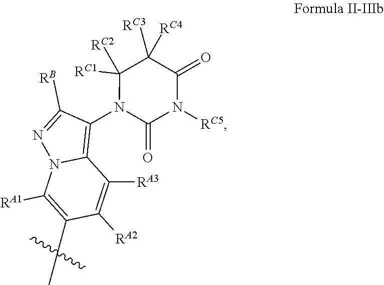

wherein:
R^{A1}, R^{A2}, and R^{A3} are each independently H, hydroxyl, halogen, (C₁-C₆)alkyl, (C₁-C₆)heteroalkyl, (C₁-C₆)alkoxy, or NH₂;
R^{B} is H or (C₁-C₆)alkyl; and
R^{C1}, R^{C2}, R^{C3}, R^{C4}, and R^{C5} are each independently H, hydroxyl, halogen, or (C₁-C₆)alkyl, (C₁-C₆)heteroalkyl, (C₁-C₆)alkoxy, or NH₂,

or a pharmaceutically acceptable salt thereof.

Embodiment 29. The compound of embodiment 28, wherein:
R^{A1}, R^{A2}, and R^{A3} are each independently H;
R^{C1}, R^{C2}, R^{C3}, R^{C4} are each independently H;
R^{C5} is H; and

or a pharmaceutically acceptable salt thereof.

Embodiment 30. The compound of any one of embodiments 1-23, 28, or 29, wherein E is

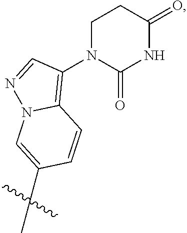 [SMILES: *c1ccc2c(N3CCC(=O)NC3=O)cnn2c1]

or a pharmaceutically acceptable salt thereof.

Embodiment 31. The compound of any one of embodiments 1-23, wherein E is of Formula II-IIIc:

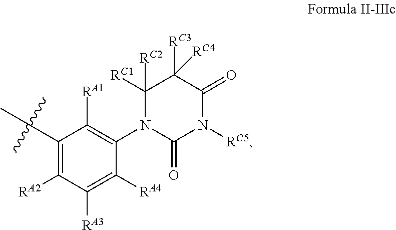

wherein:
R^{A1}, R^{A2}, R^{A3} and R^{A4} are each independently H, hydroxyl, halogen, (C₁-C₆)alkyl, (C₁-C₆)heteroalkyl, (C₁-C₆)alkoxy, or NH₂; and
R^{C1}, R^{C2}, R^{C3}, R^{C4}, and R^{C5} are each independently H, hydroxyl, halogen, (C₁-C₆)alkyl, (C₁-C₆)heteroalkyl, (C₁-C₆)alkoxy, or NH₂,

or a pharmaceutically acceptable salt thereof.

Embodiment 32. The compound of embodiment 31, wherein:
R^{A1}, R^{A2}, and R^{A3} are each independently H;
R^{A4} is (C₁-C₃)alkyl or halogen; and
R^{C1}, R^{C2}, R^{C3}, R^{C4}, and R^{C5} are each independently H,

or a pharmaceutically acceptable salt thereof.

Embodiment 33. The compound of any one of embodiments 1-23, 31, or 32, wherein E is

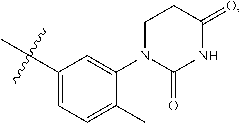 [SMILES: *c1ccc(C)c(N2CCC(=O)NC2=O)c1]

or a pharmaceutically acceptable salt thereof.

Embodiment 34. The compound of any one of embodiments 1-23, wherein E is of Formula II-IIId:

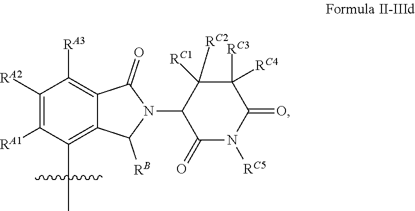

wherein:
R^{A1}, R^{A2}, and R^{A3} are each independently H, hydroxyl, halogen, (C₁-C₆)alkyl, (C₁-C₆)heteroalkyl, (C₁-C₆)alkoxy, or NH₂;
R^{B} is H or (C₁-C₆)alkyl; and
R^{C1}, R^{C2}, R^{C3}, R^{C4}, and R^{C5} are each independently H, hydroxyl, halogen, (C₁-C₆)alkyl, (C₁-C₆)heteroalkyl, (C₁-C₆)alkoxy, or NH₂,

or a pharmaceutically acceptable salt thereof.

Embodiment 35. The compound of embodiment 34, wherein:
R^{A1}, R^{A2}, and R^{A3} are each independently H;
R^{B} is H; and
R^{C1}, R^{C2}, R^{C3}, R^{C4}, and R^{C5} are each independently H,

or a pharmaceutically acceptable salt thereof.

Embodiment 36. The compound of any one of embodiments 1-23, 34, or 35, wherein E is

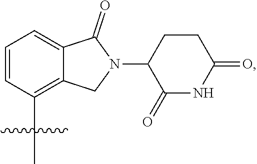 [SMILES: *c1cccc2c1CN(C1CCC(=O)NC1=O)C2=O]

or a pharmaceutically acceptable salt thereof.

Embodiment 37. The compound of any one of embodiments 1-23, wherein E is of Formula II-IIIe:

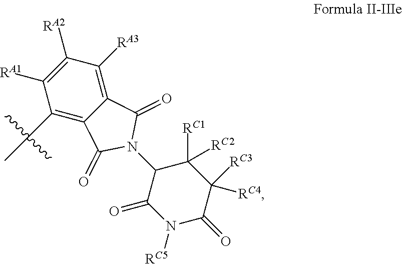

wherein:
R^{A1}, R^{A2}, and R^{A3} are each independently H, hydroxyl, halogen, (C₁-C₆)alkyl, (C₁-C₆)heteroalkyl, (C₁-C₆)alkoxy, or NH₂; and
R^{C1}, R^{C2}, R^{C3}, R^{C4}, and R^{C5} are each independently H, hydroxyl, halogen, (C₁-C₆)alkyl, (C₁-C₆)heteroalkyl, (C₁-C₆)alkoxy, or NH₂,

or a pharmaceutically acceptable salt thereof.

Embodiment 38. The compound of embodiment 38, wherein E is

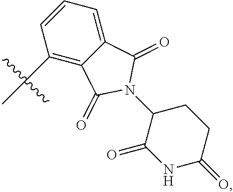 [SMILES: *c1cccc2c1C(=O)N(C1CCC(=O)NC1=O)C2=O]

or a pharmaceutically acceptable salt thereof.

Embodiment 39. The compound of embodiment 1, selected from the group consisting of:
N-{[(1r,4r)-4-{6-[2-(3-{1-[(3RS)-2,6-dioxopiperidin-3-yl]-3-methyl-2-oxo-2,3-dihydro-1H-benzimidazol-5-yl}propyl)-2,3-dihydro-1H-isoindol-5-yl]-2H-indazol-2-yl}cyclohexyl]methyl}-2,3,5-trifluoro-4-hydroxybenzamide;
N-{[4-(7-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-5-yl}imidazo[1,2-a]pyridin-2-yl)bicyclo[2.2.2]octan-1-yl]methyl}-2,3,5-trifluoro-4-hydroxybenzamide;
N-{[(1r,4r)-4-(6-{2-[8-(3-{1-[(3RS)-2,6-dioxopiperidin-3-yl]-3-methyl-2-oxo-2,3-dihydro-1H-benzimidazol-5-yl}propyl)-5-oxa-2,8-diazaspiro[3.5]nonan-2-yl]pyrimidin-5-yl}-2H-indazol-2-yl)cyclohexyl]methyl}-2,3,5-trifluoro-4-hydroxybenzamide;
N-{[(1r,4r)-4-{6-[6-(3-{1-[(3RS)-2,6-dioxopiperidin-3-yl]-3-methyl-2-oxo-2,3-dihydro-1H-benzimidazol-5-yl}propyl)-6,7-dihydro-5H-pyrrolo[3,4-b]pyridin-2-yl]-2H-indazol-2-yl}cyclohexyl]methyl}-2,3,5-trifluoro-4-hydroxybenzamide;
N-{[4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)bicyclo[2.2.2]octan-1-yl]methyl}-2,3,5-trifluoro-4-hydroxybenzamide;
N-{[(1r,4r)-4-(6-{5-[4-(2-{1-[(3RS)-2,6-dioxopiperidin-3-yl]-3-methyl-2-oxo-2,3-dihydro-1H-benzimidazol-5-yl}ethyl)piperazin-1-yl]pyrazin-2-yl}-2H-indazol-2-yl)cyclohexyl]methyl}-2,3,5-trifluoro-4-hydroxybenzamide;
N-{[(1r,4r)-4-(6-{6-[4-(2-{1-[(3RS)-2,6-dioxopiperidin-3-yl]-3-methyl-2-oxo-2,3-dihydro-1H-benzimidazol-5-yl}ethyl)piperazin-1-yl]pyrazin-2-yl}-2H-indazol-2-yl)cyclohexyl]methyl}-2,3,5-trifluoro-4-hydroxybenzamide;
N-{[(1r,4r)-4-{6-[4-(2-{1-[(3RS)-2,6-dioxopiperidin-3-yl]-3-methyl-2-oxo-2,3-dihydro-1H-benzimidazol-5-yl}ethyl)piperazin-1-yl]-2H-indazol-2-yl}cyclohexyl]methyl}-2,3,5-trifluoro-4-hydroxybenzamide;
N-{[(1r,4r)-4-{6-[5-(4-{3-[3-(2,4-dioxo-1,3-diazinan-1-yl)pyrazolo[1,5-a]pyridin-6-yl]propyl}piperazin-1-yl)pyrazin-2-yl]-2H-indazol-2-yl}cyclohexyl]methyl}-2,3,5-trifluoro-4-hydroxybenzamide, hydrochloride salt;
N-{[4-(4-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,3-thiazol-2-yl)bicyclo[2.2.2]octan-1-yl]methyl}-2,3,5-trifluoro-4-hydroxybenzamide;
N-{[4-(3-{6-[4-(3-{1-[(3RS)-2,6-dioxopiperidin-3-yl]-3-methyl-2-oxo-2,3-dihydro-1H-benzimidazol-5-yl}propyl)piperazin-1-yl]pyridazin-3-yl}-1,2,4-oxadiazol-5-yl)bicyclo[2.2.2]octan-1-yl]methyl}-2,3,5-trifluoro-4-hydroxybenzamide;
N-{[4-(2-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,3-thiazol-4-yl)bicyclo[2.2.2]octan-1-yl]methyl}-2,3,5-trifluoro-4-hydroxybenzamide;
N-{[(1r,4r)-4-{5-[4-(3-{1-[(3RS)-2,6-dioxopiperidin-3-yl]-3-methyl-2-oxo-2,3-dihydro-1H-benzimidazol-5-yl}propyl)piperazin-1-yl]-2H-pyrazolo[4,3-b]pyridin-2-yl}cyclohexyl]methyl}-2,3,5-trifluoro-4-hydroxybenzamide;
N-{[(1r,4r)-4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)cyclohexyl]methyl}-2,3,5-trifluoro-4-hydroxybenzamide;
N-{[4-(2-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,3-oxazol-5-yl)bicyclo[2.2.2]octan-1-yl]methyl}-2,3,5-trifluoro-4-hydroxybenzamide;
N-{[(1r,4r)-4-{6-[2-(4-{3-[3-(2,4-dioxo-1,3-diazinan-1-yl)imidazo[1,2-a]pyridin-7-yl]propyl}piperazin-1-yl)pyrimidin-5-yl]-2H-indazol-2-yl}cyclohexyl]methyl}-2,3,5-trifluoro-4-hydroxybenzamide;
N-{[(1r,4r)-4-{6-[2-(4-{8-[3-(2,4-dioxo-1,3-diazinan-1-yl)-4-methylbenzoyl]-1-oxa-8-azaspiro[4.5]decan-3-yl}piperazin-1-yl)pyrimidin-5-yl]-2H-indazol-2-yl}cyclohexyl]methyl}-2,3,5-trifluoro-4-hydroxybenzamide; and
N-{[(1r,4r)-4-{6-[4-({4-[({2-[(3RS)-2,6-dioxopiperidin-3-yl]-1-oxo-2,3-dihydro-1H-isoindol-4-yl}oxy)methyl]phenyl}methyl)piperazin-1-yl]-2H-indazol-2-yl}cyclohexyl]methyl}-3,5-difluoro-4-hydroxybenzamide;
N-{[(1r,4r)-4-{6-[2-(2-{4-[3-(2,4-dioxo-1,3-diazinan-1-yl)-4-methylbenzoyl]piperazin-1-yl}ethoxy)pyrimidin-5-yl]-2H-indazol-2-yl}cyclohexyl]methyl}-2,3,5-trifluoro-4-hydroxybenzamide,

or a pharmaceutically acceptable salt thereof.

Embodiment 40. A compound of the structure:

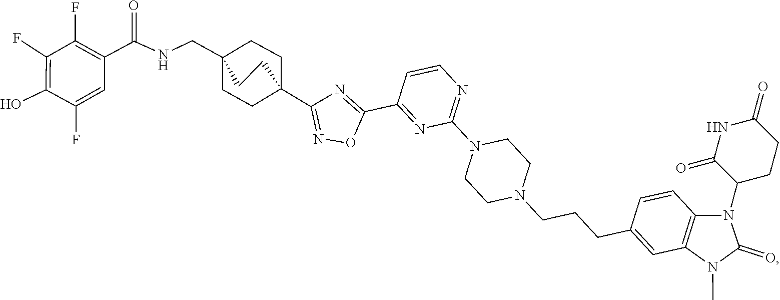 [SMILES: Cn1c(=O)n(C2CCC(=O)NC2=O)c2ccc(CCCN3CCN(c4nccc(-c5nc([C@]67CC[C@](CNC(=O)c8cc(F)c(O)c(F)c8F)(CC6)CC7)no5)n4)CC3)cc21]

or a pharmaceutically acceptable salt thereof.

Embodiment 41. A pharmaceutically acceptable salt of N-{[4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)bicyclo[2.2.2]octan-1-yl]methyl}-2,3,5-trifluoro-4-hydroxybenzamide.

Embodiment 42. N-{[4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)bicyclo[2.2.2]octan-1-yl]methyl}-2,3,5-trifluoro-4-hydroxybenzamide, hydrochloride salt.

Embodiment 43. N-{[4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)bicyclo[2.2.2]octan-1-yl]methyl}-2,3,5-trifluoro-4-hydroxybenzamide.

Embodiment 44. A pharmaceutical composition comprising a therapeutically effective amount of a compound of any one of embodiments 1 to 43 or a pharmaceutically acceptable salt thereof, and at least one pharmaceutically acceptable carrier, vehicle, or diluent.

Embodiment 45. A pharmaceutical combination composition comprising: a therapeutically effective amount of a composition comprising:
a first compound, said first compound being a compound of any one of embodiments 1-43 or a pharmaceutically acceptable salt of said compound;
a second compound, said second compound being an anti-diabetic agent; a non-alcoholic steatohepatitis treatment agent, a non-alcoholic fatty liver disease treatment agent or an anti-heart failure treatment agent and
a pharmaceutical carrier, vehicle, or diluents.

Embodiment 46. The pharmaceutical combination composition of embodiment 45, wherein the non-alcoholic steatohepatitis treatment agent or non-alcoholic fatty liver disease treatment agent is an ACC inhibitor, a KHK inhibitor, a DGAT-2 inhibitor, an FXR agonist, metformin, incretin analogues, or an incretin receptor modulator.

Embodiment 47. The pharmaceutical combination composition of embodiment 45, wherein the anti-diabetic agent is an SGLT-2 inhibitor, metformin, incretin analogues, an incretin receptor modulator, a DPP-4 inhibitor, or a PPAR agonist.

Embodiment 48. A method for treating a condition, comprising administering to a subject in need thereof a therapeutically effective amount of the compound of any one of embodiments 1-43, or a pharmaceutically acceptable salt thereof, wherein the condition is selected from the group consisting of: fatty liver, non-alcoholic fatty liver disease, non-alcoholic steatohepatitis, non-alcoholic steatohepatitis with liver fibrosis, non-alcoholic steatohepatitis with cirrhosis, non-alcoholic steatohepatitis with cirrhosis, hepatocellular carcinoma, alcoholic fatty liver disease, alcoholic steatohepatitis, hepatitis B, hepatitis C, biliary cirrhosis, kidney renal clear cell carcinoma, head and neck squamous cell carcinoma, colorectal adenocarcinoma, mesothelioma, stomach adenocarcinoma, adrenocortical carcinoma, kidney papillary cell carcinoma, cervical and endocervical carcinoma, bladder urothelial carcinoma, lung adenocarcinoma, Type I diabetes, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, restenosis after angioplasty, peripheral vascular disease, intermittent claudication, post-prandial lipemia, metabolic acidosis, ketosis, arthritis, diabetic retinopathy, macular degeneration, cataract, diabetic nephropathy, glomerulosclerosis, chronic renal failure, diabetic neuropathy, skin and connective tissue disorders, foot ulcerations and ulcerative colitis, endothelial dysfunction and impaired vascular compliance, kidney disease, end-stage kidney disease, chronic kidney disease at risk of progression, and maple syrup urine disease.

Embodiment 49. The method of embodiment 48, wherein the condition is alcoholic fatty liver disease, alcoholic steatohepatitis, hepatitis B, hepatitis C, or biliary cirrhosis.

Embodiment 50. The method of embodiment 48, wherein the condition is fatty liver, non-alcoholic fatty liver disease, non-alcoholic steatohepatitis, non-alcoholic steatohepatitis with liver fibrosis, non-alcoholic steatohepatitis with cirrhosis or non-alcoholic steatohepatitis with cirrhosis or hepatocellular carcinoma.

Embodiment 51. The method of embodiment 48, wherein the condition is non-alcoholic steatohepatitis.

Embodiment 52. A method of reducing development of a condition, comprising administering to a subject in need thereof a therapeutically effective amount of a compound of any one of embodiments 1-43 or a pharmaceutically acceptable salt thereof, wherein the condition is selected from the group consisting of: liver cirrhosis, cirrhotic decompensation, progression to model of end-stage liver disease (MELD), liver transplant, liver-related death, and hepatocellular carcinoma.

Embodiment 53. A compound according to any one of embodiments 1-43 or a pharmaceutically acceptable salt thereof, for use as a medicament.

Embodiment 54. A compound according to any one of embodiments 1-43 or a pharmaceutically acceptable salt thereof, for use in the treatment of fatty liver, non-alcoholic fatty liver disease, non-alcoholic steatohepatitis, non-alcoholic steatohepatitis with liver fibrosis, non-alcoholic steatohepatitis with cirrhosis, non-alcoholic steatohepatitis with cirrhosis, hepatocellular carcinoma, alcoholic fatty liver disease, alcoholic steatohepatitis, hepatitis B, hepatitis C, biliary cirrhosis, kidney renal clear cell carcinoma, head and neck squamous cell carcinoma, colorectal adenocarcinoma, mesothelioma, stomach adenocarcinoma, adrenocortical carcinoma, kidney papillary cell carcinoma, cervical and endocervical carcinoma, bladder urothelial carcinoma, lung adenocarcinoma, Type I diabetes, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, restenosis after angioplasty, peripheral vascular disease, intermittent claudication, post-prandial lipemia, metabolic acidosis, ketosis, arthritis, diabetic retinopathy, macular degeneration, cataract, diabetic nephropathy, glomerulosclerosis, chronic renal failure, diabetic neuropathy, skin and connective tissue disorders, foot ulcerations and ulcerative colitis, endothelial dysfunction and impaired vascular compliance, kidney disease, end-stage kidney disease, chronic kidney disease at risk of progression, or maple syrup urine disease.

Embodiment 55. Use of a compound according to any one of embodiments 1-43 or a pharmaceutically acceptable salt thereof, in the manufacture of a medicament for the treatment of fatty liver, non-alcoholic fatty liver disease, non-alcoholic steatohepatitis, non-alcoholic steatohepatitis with liver fibrosis, non-alcoholic steatohepatitis with cirrhosis, non-alcoholic steatohepatitis with cirrhosis, hepatocellular carcinoma, alcoholic fatty liver disease, alcoholic steatohepatitis, hepatitis B, hepatitis C, biliary cirrhosis, kidney renal clear cell carcinoma, head and neck squamous cell carcinoma, colorectal adenocarcinoma, mesothelioma, stomach adenocarcinoma, adrenocortical carcinoma, kidney papillary cell carcinoma, cervical and endocervical carcinoma, bladder urothelial carcinoma, lung adenocarcinoma, Type I diabetes, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, restenosis after angioplasty, peripheral vascular disease, intermittent claudication, post-prandial lipemia, metabolic acidosis, ketosis, arthritis, diabetic retinopathy, macular degeneration, cataract, diabetic nephropathy, glomerulosclerosis, chronic renal failure, diabetic neuropathy, skin and connective tissue disorders, foot ulcerations and ulcerative colitis, endothelial dysfunction and impaired vascular compliance, kidney disease, end-stage kidney disease, chronic kidney disease at risk of progression, or maple syrup urine disease.

Embodiment 56. Use of a compound according to any one of embodiments 1-43 or a pharmaceutically acceptable salt thereof, as a medicament.

Embodiment 57. Use of a compound according to any one of embodiments 1-43 or a pharmaceutically acceptable salt thereof, in treating a condition selected from the group consisting of: fatty liver, non-alcoholic fatty liver disease, non-alcoholic steatohepatitis, non-alcoholic steatohepatitis with liver fibrosis, non-alcoholic steatohepatitis with cirrhosis, non-alcoholic steatohepatitis with cirrhosis, hepatocellular carcinoma, alcoholic fatty liver disease, alcoholic steatohepatitis, hepatitis B, hepatitis C, biliary cirrhosis, kidney renal clear cell carcinoma, head and neck squamous cell carcinoma, colorectal adenocarcinoma, mesothelioma, stomach adenocarcinoma, adrenocortical carcinoma, kidney papillary cell carcinoma, cervical and endocervical carcinoma, bladder urothelial carcinoma, lung adenocarcinoma, Type I diabetes, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, restenosis after angioplasty, peripheral vascular disease, intermittent claudication, post-prandial lipemia, metabolic acidosis, ketosis, arthritis, diabetic retinopathy, macular degeneration, cataract, diabetic nephropathy, glomerulosclerosis, chronic renal failure, diabetic neuropathy, skin and connective tissue disorders, foot ulcerations and ulcerative colitis, endothelial dysfunction and impaired vascular compliance, kidney disease, end-stage kidney disease, chronic kidney disease at risk of progression, and maple syrup urine disease.

Each of the embodiments described herein may be combined with any other embodiment(s) described herein not inconsistent with the embodiment(s) with which it is combined. In addition, any of the compounds described in the Examples, or pharmaceutically acceptable salts thereof, may be claimed individually or grouped together with one or more other compounds of the Examples, or pharmaceutically acceptable salts thereof, for any of the embodiment(s) described herein. Furthermore, each of the embodiments described herein envisions within its scope pharmaceutically acceptable salts of the compounds described herein.

It will be apparent to those skilled in the art that various modifications and variations may be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All references cited herein, including patents, patent applications, papers, textbooks, and the like, and the references cited therein, to the extent that they are not already, are hereby incorporated by reference in their entireties. In the event that one or more of the incorporated literature and similar materials differs from or contradicts this application, including but not limited to defined terms, term usage, described techniques, or the like, this application controls.

1. A compound of Formula II: 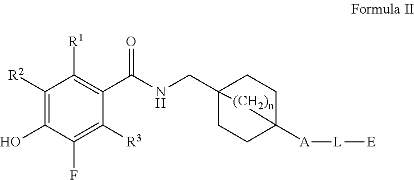
wherein:
A is —NH—C(O)—, —C(O)—, or heteroaryl, wherein heteroaryl has 1, 2, 3, or 4 heteroatoms selected from O, N, and S, and wherein A is optionally substituted with one or two R⁴;
R¹, R², and R³ are each independently selected from H and fluoro;
R⁴ is selected from oxo, hydroxyl, chloro, fluoro, (C₁-C₆)alkyl, (C₁-C₆)alkoxy, (C₁-C₆)fluoroalkyl, (C₃-C₆)cycloalkyl, and heterocyclyl, wherein the heterocyclyl has 1, 2, or 3 heteroatoms selected from O and N;
n is 0, 1, or 2;
L is a linker; and
E is an E3 ubiquitin ligase binder,
or a pharmaceutically acceptable salt thereof.

2. The compound of claim 1, wherein the compound has the Formula IIB: 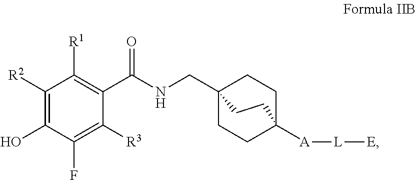
or a pharmaceutically acceptable salt thereof.

3. The compound of claim 1, wherein A is thiazolyl, pyrazolyl, oxazolyl, imidazolyl, isoxazolyl, isothiazolyl, imidazotriazinyl, imidazopyridazinyl, imidazopyridinyl, benzoimidazolyl, benzothiazolyl, purinyl, pyridopyridazinyl, quinazolinyl, indazolyl, imidazopyridinyl, benzooxazolyl, pyrazolopyridinyl, isoindolinonyl, triazolyl, or oxadiazolyl, or a pharmaceutically acceptable salt thereof.

4. The compound of claim 1, wherein A is: 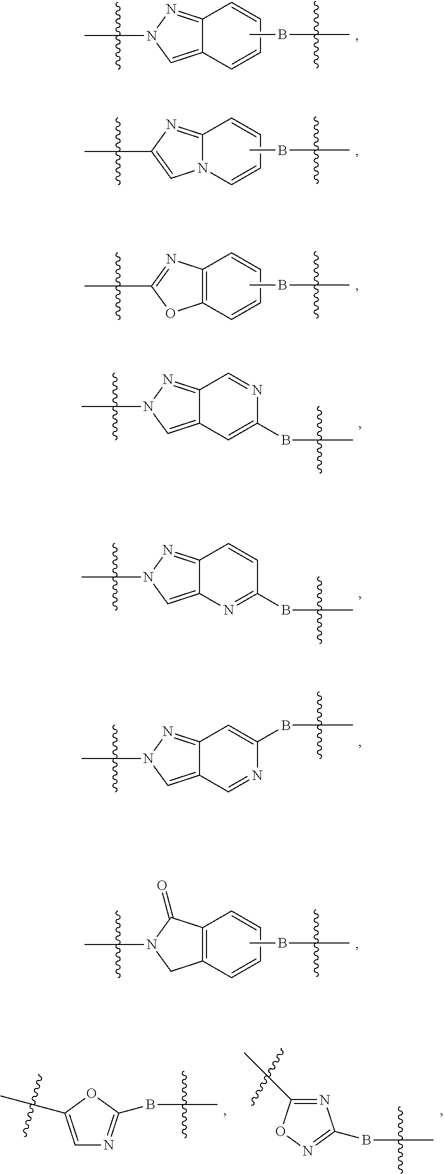 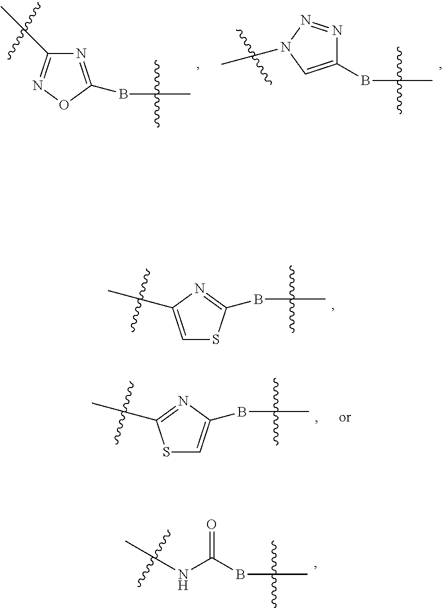
or a pharmaceutically acceptable salt thereof.

5. The compound of claim 1, wherein L is of the formula: 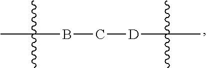
wherein:
B is absent; or aryl, heteroaryl, heterocyclyl, —C(O)—, (C₁-C₆)alkylene, (C₃-C₆)cycloalkylene, (C₁-C₆)fluoroalkylene, (C₁-C₆)alkoxy, or (C₁-C₆)fluoroalkoxy, wherein the heteroaryl, or heterocyclyl has 1, 2, or 3 heteroatoms selected from O, N, and S, and wherein B is optionally substituted with one or two R⁵;
C is absent; or —NH—C(O)—R⁷, —S(O)₂—R⁷, —O—S(O)₂—R⁷, —C(O)—, (C₁-C₆)alkylene, (C₁-C₆)aminoalkylene, (C₃-C₆)cycloalkylene, (C₁-C₆)alkoxy, (C₃-C₆)cycloether, (C₁-C₆)fluoroalkylene, (C₁-C₆)fluoroalkoxy, aryl, heteroaryl, or heterocyclyl, wherein the heteroaryl or heterocyclyl has 1, 2, or 3 heteroatoms selected from O, N, and S, and wherein C is optionally substituted with one, two or three R⁶;
D is (C₁-C₆)alkylene, (C₁-C₆)aminoalkylene, —NH(C₁-C₆)alkylene, (C₁-C₆)alkoxy, —C(O)—, aryl, heteroaryl, heterocyclyl, (C₀-C₆)alkylene-heterocyclyl-C(O)—, —C(O)—(C₁-C₆)alkylene, heterocyclyl-(C₁-C₆)alkylene-aryl-(C₁-C₆)alkoxy, (C₁-C₆)heterocyclyl-(C₁-C₆)heterocyclyl-C(O)—, (C₀-C₂)alkylene-aryl-(C₁-C₆)alkoxy, —O-heterocyclyl-C(O)—, (C₁-C₆)cycloalkyl-(C₁-C₆)heterocyclyl, wherein the heteroaryl or heterocyclyl has 1, 2, or 3 heteroatoms selected from O, N, and S, wherein D is optionally substituted with one or two R⁸; or a bond;
R⁵, R⁶, and R⁸ are each independently selected from oxo, hydroxyl, halogen, (C₁-C₆)alkyl, (C₁-C₆)alkoxy, (C₁-C₆)fluoroalkyl, (C₃-C₆)cycloalkyl, heteroaryl, and heterocyclyl, wherein the heterocyclyl has 1, 2, or 3 heteroatoms selected from O and N;
R⁷ is (C₁-C₆)alkyl, (C₁-C₆)alkoxy, (C₁-C₆)fluoroalkyl, or (C₃-C₆)cycloalkyl;
or a pharmaceutically acceptable salt thereof.

6. The compound of claim 5, wherein B is pyridinyl, pyrimidinyl, pyridazinyl, pyrazinyl, pyrazolyl, piperazinyl, quinoxalinyl, phenyl, triazolyl, thiazolyl, thiadiazolyl, oxazolyl, imidazolyl, indazolyl, (C₁-C₆)alkylene, (C₁-C₆)fluoroalkylene, (C₁-C₆)alkoxy, pyrrolopyridinyl, isoindolinyl, isoquinolinyl, tetrahydroisoquinolinyl, thiazolopyridinyl, tetrahydrothiazolopyridinyl, imidazopyrazinyl, tetrahydroimidazopyrazinyl, pyrazolopyrazinyl, tetrahydropyrazolopyrazinyl, phenyl, oxadiazaspirodecanyl, diazaspirooctanyl, or diazaspirodecan-1-only, wherein B is optionally substituted with one or two halogen, oxo, hydroxyl, (C₁-C₆)alkyl, (C₃-C₆)cycloalkyl, (C₁-C₆)fluoroalkyl, (C₁-C₆)alkoxy, or (C₃-C₆)cycloether, or a pharmaceutically acceptable salt thereof.

7. The compound of claim 5, wherein C is (C₁-C₃)alkylene, (C₁-C₆)aminoalkylene, (C₁-C₆)alkoxy, pyridinyl, oxolanyl, (C₃-C₆)cycloalkyl, (C₁-C₆)fluoroalkylene, —C(O)—, piperazinyl, piperidinyl, azetidinyl, azaspiroundecanyl, azaspirononanyl, azaspiroundecanyl, diazaspirooctanyl, diazaspirodecanyl, diazaspirononanyl, diazaspirododecanyl, diazaspiroundecanyl, oxadiazaspirononanyl, oxadiazaspiroundecanyl, oxa-azaspirodecanyl, decahydronaphthyridinyl, octahydropyrrolopyridinyl, or octahydropyridopyrazinyl; wherein C is optionally substituted with one, two or three halogen, oxo, hydroxyl, (C₁-C₆)alkyl, (C₃-C₆)cycloalkyl, (C₁-C₆)fluoroalkyl, or (C₁-C₆)alkoxy, or a pharmaceutically acceptable salt thereof.

8. The compound of claim 5, wherein D is (C₁-C₆)alkylene, (C₁-C₆)aminoalkylene, (C₁-C₆)alkoxy, —C(O)—, (C₀-C₆)alkylene-heterocyclyl-C(O)—, —C(O)—(C₁-C₆)alkylene, heterocyclyl-(C₁-C₆)alkylene-aryl-(C₁-C₆)alkoxy, (C₁-C₆)heterocyclyl-(C₁-C₆)heterocyclyl-C(O)—, (C₀-C₂)alkylene-aryl-(C₁-C₆)alkoxy, —O-heterocyclyl-C(O)—, (C₁-C₆)cycloalkyl-(C₁-C₆)heterocyclyl; or a bond, or a pharmaceutically acceptable salt thereof.

9. The compound of claim 5, wherein:
A is heteroaryl;
B is heteroaryl;
C is heterocyclyl; and
D is (C₁-C₃)alkylene,
or a pharmaceutically acceptable salt thereof.

10. The compound of claim 5, wherein:
A is indazolyl or oxadiazolyl;
B is pyrimidinyl;
C is piperazinyl; and
D is methylene, ethylene, or propylene,
or a pharmaceutically acceptable salt thereof.

11. The compound of claim 1, wherein E comprises a benzimidazolinone, a dihydropyrimidine-dione, or a thalidomide; or a pharmaceutically acceptable salt thereof.

12. The compound of claim 1, wherein E is selected from the group consisting of: 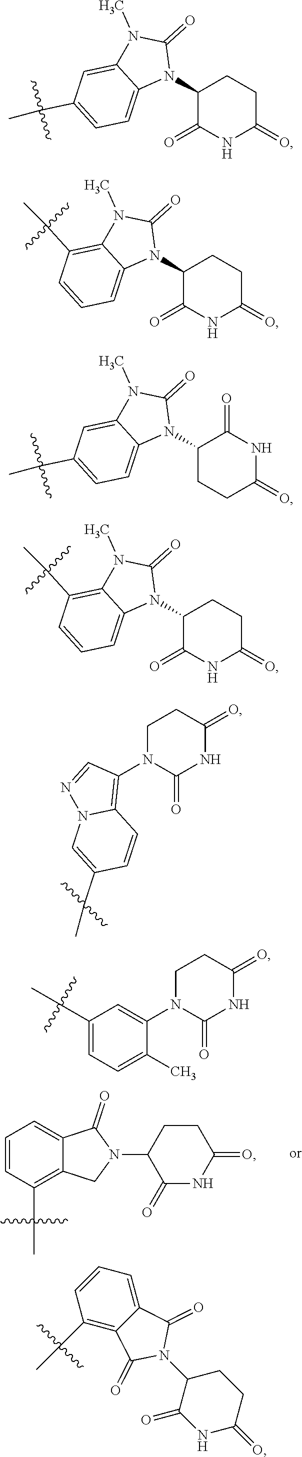
or a pharmaceutically acceptable salt thereof.

13. The compound of claim 1, selected from the group consisting of:
N-{[(1r,4r)-4-{6-[2-(3-{1-[(3RS)-2,6-dioxopiperidin-3-yl]-3-methyl-2-oxo-2,3-dihydro-1H-benzimidazol-5-yl}propyl)-2,3-dihydro-1H-isoindol-5-yl]-2H-indazol-2-yl}cyclohexyl]methyl}-2,3,5-trifluoro-4-hydroxybenzamide;
N-{[4-(7-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-5-yl}imidazo[1,2-a]pyridin-2-yl)bicyclo[2.2.2]octan-1-yl]methyl}-2,3,5-trifluoro-4-hydroxybenzamide;
N-{[(1r,4r)-4-(6-{2-[8-(3-{1-[(3RS)-2,6-dioxopiperidin-3-yl]-3-methyl-2-oxo-2,3-dihydro-1H-benzimidazol-5-yl}propyl)-5-oxa-2,8-diazaspiro[3.5]nonan-2-yl]pyrimidin-5-yl}-2H-indazol-2-yl)cyclohexyl]methyl}-2,3,5-trifluoro-4-hydroxybenzamide;
N-{[(1r,4r)-4-{6-[6-(3-{1-[(3RS)-2,6-dioxopiperidin-3-yl]-3-methyl-2-oxo-2,3-dihydro-1H-benzimidazol-5-yl}propyl)-6,7-dihydro-5H-pyrrolo[3,4-b]pyridin-2-yl]-2H-indazol-2-yl}cyclohexyl]methyl}-2,3,5-trifluoro-4-hydroxybenzamide;
N-{[4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)bicyclo[2.2.2]octan-1-yl]methyl}-2,3,5-trifluoro-4-hydroxybenzamide;
N-{[(1r,4r)-4-(6-{5-[4-(2-{1-[(3RS)-2,6-dioxopiperidin-3-yl]-3-methyl-2-oxo-2,3-dihydro-1H-benzimidazol-5-yl}ethyl)piperazin-1-yl]pyrazin-2-yl}-2H-indazol-2-yl)cyclohexyl]methyl}-2,3,5-trifluoro-4-hydroxybenzamide;
N-{[(1r,4r)-4-(6-{6-[4-(2-{1-[(3RS)-2,6-dioxopiperidin-3-yl]-3-methyl-2-oxo-2,3-dihydro-1H-benzimidazol-5-yl}ethyl)piperazin-1-yl]pyrazin-2-yl}-2H-indazol-2-yl)cyclohexyl]methyl}-2,3,5-trifluoro-4-hydroxybenzamide;
N-{[(1r,4r)-4-{6-[4-(2-{1-[(3RS)-2,6-dioxopiperidin-3-yl]-3-methyl-2-oxo-2,3-dihydro-1H-benzimidazol-5-yl}ethyl)piperazin-1-yl]-2H-indazol-2-yl}cyclohexyl]methyl}-2,3,5-trifluoro-4-hydroxybenzamide;
N-{[(1r,4r)-4-{6-[5-(4-{3-[3-(2,4-dioxo-1,3-diazinan-1-yl)pyrazolo[1,5-a]pyridin-6-yl]propyl}piperazin-1-yl)pyrazin-2-yl]-2H-indazol-2-yl}cyclohexyl]methyl}-2,3,5-trifluoro-4-hydroxybenzamide, hydrochloride salt;
N-{[4-(4-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,3-thiazol-2-yl)bicyclo[2.2.2]octan-1-yl]methyl}-2,3,5-trifluoro-4-hydroxybenzamide;
N-{[4-(3-{6-[4-(3-{1-[(3RS)-2,6-dioxopiperidin-3-yl]-3-methyl-2-oxo-2,3-dihydro-1H-benzimidazol-5-yl}propyl)piperazin-1-yl]pyridazin-3-yl}-1,2,4-oxadiazol-5-yl)bicyclo[2.2.2]octan-1-yl]methyl}-2,3,5-trifluoro-4-hydroxybenzamide;
N-{[4-(2-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,3-thiazol-4-yl)bicyclo[2.2.2]octan-1-yl]methyl}-2,3,5-trifluoro-4-hydroxybenzamide;
N-{[(1r,4r)-4-{5-[4-(3-{1-[(3RS)-2,6-dioxopiperidin-3-yl]-3-methyl-2-oxo-2,3-dihydro-1H-benzimidazol-5-yl}propyl)piperazin-1-yl]-2H-pyrazolo[4,3-b]pyridin-2-yl}cyclohexyl]methyl}-2,3,5-trifluoro-4-hydroxybenzamide;
N-{[(1r,4r)-4-(5-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,2,4-oxadiazol-3-yl)cyclohexyl]methyl}-2,3,5-trifluoro-4-hydroxybenzamide;
N-{[4-(2-{2-[4-(3-{1-[(3RS)-2,6-dioxopiperidin-3-yl]-3-methyl-2-oxo-2,3-dihydro-1H-benzimidazol-5-yl}propyl)piperazin-1-yl]pyrimidin-4-yl}-1,3-oxazol-5-yl)bicyclo[2.2.2]octan-1-yl]methyl}-2,3,5-trifluoro-4-hydroxybenzamide;
N-{[(1r,4r)-4-{6-[2-(4-{3-[3-(2,4-dioxo-1,3-diazinan-1-yl)imidazo[1,2-a]pyridin-7-yl]propyl}piperazin-1-yl)pyrimidin-5-yl]-2H-indazol-2-yl}cyclohexyl]methyl}-2,3,5-trifluoro-4-hydroxybenzamide;
N-{[(1r,4r)-4-{6-[2-(4-{8-[3-(2,4-dioxo-1,3-diazinan-1-yl)-4-methylbenzoyl]-1-oxa-8-azaspiro[4.5]decan-3-yl}piperazin-1-yl)pyrimidin-5-yl]-2H-indazol-2-yl}cyclohexyl]methyl}-2,3,5-trifluoro-4-hydroxybenzamide; and
N-{[(1r,4r)-4-{6-[4-({4-[({2-[(3RS)-2,6-dioxopiperidin-3-yl]-1-oxo-2,3-dihydro-1H-isoindol-4-yl}oxy)methyl]phenyl}methyl)piperazin-1-yl]-2H-indazol-2-yl}cyclohexyl]methyl}-3,5-difluoro-4-hydroxybenzamide;
N-{[(1r,4r)-4-{6-[2-(2-{4-[3-(2,4-dioxo-1,3-diazinan-1-yl)-4-methylbenzoyl]piperazin-1-yl}ethoxy)pyrimidin-5-yl]-2H-indazol-2-yl}cyclohexyl]methyl}-2,3,5-trifluoro-4-hydroxybenzamide,
or a pharmaceutically acceptable salt thereof.

14. A pharmaceutical composition comprising a therapeutically effective amount of a compound of claim 1 or a pharmaceutically acceptable salt thereof, and at least one pharmaceutically acceptable carrier, vehicle, or diluent.

15. A method of treating a condition, wherein the condition is selected from the group consisting of: fatty liver, non-alcoholic fatty liver disease, non-alcoholic steatohepatitis, non-alcoholic steatohepatitis with liver fibrosis, non-alcoholic steatohepatitis with cirrhosis, non-alcoholic steatohepatitis with cirrhosis, hepatocellular carcinoma, alcoholic fatty liver disease, alcoholic steatohepatitis, hepatitis B, hepatitis C, biliary cirrhosis, kidney renal clear cell carcinoma, head and neck squamous cell carcinoma, colorectal adenocarcinoma, mesothelioma, stomach adenocarcinoma, adrenocortical carcinoma, kidney papillary cell carcinoma, cervical and endocervical carcinoma, bladder urothelial carcinoma, lung adenocarcinoma, Type I diabetes, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, restenosis after angioplasty, peripheral vascular disease, intermittent claudication, post-prandial lipemia, metabolic acidosis, ketosis, arthritis, diabetic retinopathy, macular degeneration, cataract, diabetic nephropathy, glomerulosclerosis, chronic renal failure, diabetic neuropathy, skin and connective tissue disorders, foot ulcerations and ulcerative colitis, endothelial dysfunction and impaired vascular compliance, kidney disease, end-stage kidney disease, chronic kidney disease at risk of progression, and maple syrup urine disease,
wherein the method comprises administering to a subject in need thereof a therapeutically effective amount of a compound of Formula II: 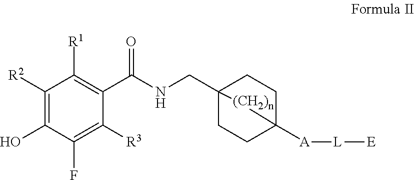
wherein:
A is —NH—C(O)—, —C(O)—, or heteroaryl, wherein heteroaryl has 1, 2, 3, or 4 heteroatoms selected from O, N, and S, and wherein A is optionally substituted with one or two R⁴;
R¹, R², and R³ are each independently selected from H and fluoro;
R⁴ is selected from oxo, hydroxyl, chloro, fluoro, (C₁-C₆)alkyl, (C₁-C₆)alkoxy, (C₁-C₆)fluoroalkyl, (C₃-C₆)cycloalkyl, and heterocyclyl, wherein the heterocyclyl has 1, 2, or 3 heteroatoms selected from O and N;
n is 0, 1, or 2;
L is a linker; and
E is an E3 ubiquitin ligase binder,
or a pharmaceutically acceptable salt thereof.

16. The method of claim 15, wherein the condition is alcoholic fatty liver disease, alcoholic steatohepatitis, hepatitis B, hepatitis C, or biliary cirrhosis.

17. A method of reducing development of a condition, comprising administering to a subject in need thereof a therapeutically effective amount of a compound of Formula II: 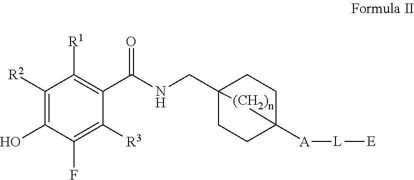
wherein:
A is —NH—C(O)—, —C(O)—, or heteroaryl, wherein heteroaryl has 1, 2, 3, or 4 heteroatoms selected from O, N, and S, and wherein A is optionally substituted with one or two R⁴;
R¹, R², and R³ are each independently selected from H and fluoro;
R⁴ is selected from oxo, hydroxyl, chloro, fluoro, (C₁-C₆)alkyl, (C₁-C₆)alkoxy, (C₁-C₆)fluoroalkyl, (C₃-C₆)cycloalkyl, and heterocyclyl, wherein the heterocyclyl has 1, 2, or 3 heteroatoms selected from O and N;
n is 0, 1, or 2;
L is a linker; and
E is an E3 ubiquitin ligase binder,
or a pharmaceutically acceptable salt thereof,
wherein the condition is selected from the group consisting of: liver cirrhosis, cirrhotic decompensation, progression to model of end-stage liver disease (MELD), liver transplant, liver-related death, and hepatocellular carcinoma.

18. A pharmaceutical combination composition comprising: a therapeutically effective amount of a composition comprising:
a) a first compound, said first compound being a compound of Formula II: 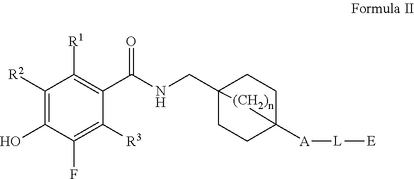
wherein:
A is —NH—C(O)—, —C(O)—, or heteroaryl, wherein heteroaryl has 1, 2, 3, or 4 heteroatoms selected from O, N, and S, and wherein A is optionally substituted with one or two R⁴;
R¹, R², and R³ are each independently selected from H and fluoro;
R⁴ is selected from oxo, hydroxyl, chloro, fluoro, (C₁-C₆)alkyl, (C₁-C₆)alkoxy, (C₁-C₆)fluoroalkyl, (C₃-C₆)cycloalkyl, and heterocyclyl, wherein the heterocyclyl has 1, 2, or 3 heteroatoms selected from O and N;
n is 0, 1, or 2;
L is a linker; and
E is an E3 ubiquitin ligase binder,
or a pharmaceutically acceptable salt thereof,
b) a second compound, said second compound being an anti-diabetic agent; a non-alcoholic steatohepatitis treatment agent, a non-alcoholic fatty liver disease treatment agent or an anti-heart failure treatment agent and
c) a pharmaceutical carrier, vehicle, or diluents.

19. The pharmaceutical combination composition of claim 18, wherein the non-alcoholic steatohepatitis treatment agent or non-alcoholic fatty liver disease treatment agent is an ACC inhibitor, a KHK inhibitor, a DGAT-2 inhibitor, an FXR agonist, metformin, incretin analogues, or an incretin receptor modulator.

20. The pharmaceutical combination composition of claim 18, wherein the anti-diabetic agent is an SGLT-2 inhibitor, metformin, incretin analogues, an incretin receptor modulator, a DPP-4 inhibitor, or a PPAR agonist.
